(19) United States
(12) Patent Application Publication
(10) Pub. No.: US 2013/0216498 A1
(43) Pub. Date: Aug. 22, 2013

(54) IMIDAZOPYRIDINE DERIVATIVES AS JAK INHIBITORS

(71) Applicants: Paul Robert Eastwood, Sant Feliu de Llobregat (ES); Jacob Gonzales Rodriguez, Sant Feliu de Llobregat (ES); Jordi Bach Taña, Sant Feliu de Llobregat (ES); Lluis Miquel Pages Santacana, Sant Feliu de Llobregat (ES); Joan Taltavull Moll, Sant Feliu de Llobregat (ES); Juan Francisco Caturla Javaloyes, Sant Feliu de Llobregat (ES); Victor Giulio Matassa, Sant Feliu de Llobregat (ES)

(72) Inventors: Paul Robert Eastwood, Sant Feliu de Llobregat (ES); Jacob Gonzales Rodriguez, Sant Feliu de Llobregat (ES); Jordi Bach Taña, Sant Feliu de Llobregat (ES); Lluis Miquel Pages Santacana, Sant Feliu de Llobregat (ES); Joan Taltavull Moll, Sant Feliu de Llobregat (ES); Juan Francisco Caturla Javaloyes, Sant Feliu de Llobregat (ES); Victor Giulio Matassa, Sant Feliu de Llobregat (ES)

(21) Appl. No.: 13/518,863

(22) PCT Filed: Dec. 23, 2010
(86) PCT No.: PCT/EP2010/007913

§ 371 Date: Sep. 21, 2012

Related U.S. Application Data
(60) Provisional application No. 61/290,293, filed on Dec. 28, 2009.

(30) Foreign Application Priority Data
Dec. 24, 2009 (EP) 09382303.7

(51) Int. Cl. C07D 471/04; A61K 31/517; A61K 31/497; A61K 31/513; A61K 31/5377; A61K 45/06; A61K 31/506; A61K 31/551

(52) U.S. Cl. CPC C07D 471/04; A61K 31/506; A61K 31/517; A61K 31/551; A61K 31/513; A61K 31/5377; A61K 45/06; A61K 31/497
USPC 424/85.6; 514/256; 544/328; 514/252.18; 544/295; 514/266.21; 544/284; 544/327; 514/218; 540/575; 544/296; 514/274; 514/269; 544/298; 514/233.2; 544/122; 544/123; 424/158.1; 424/172.1; 424/85.7; 544/405; 514/255.05; 544/319; 514/171; 424/144.1; 514/167

(57) ABSTRACT

New imidazopyridine derivatives having the chemical structure of formula (I) are disclosed; as well as process for their preparation, pharmaceutical compositions comprising them and their use in therapy as inhibitors of Janus Kinases (JAK).

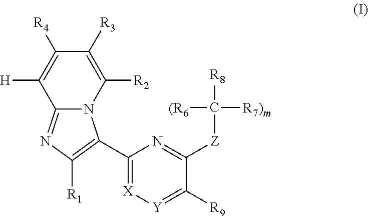

Cytokines have critical functions in regulating many aspects of immunity and inflammation, ranging from the development and differentiation of immune cells to the suppression of immune responses. Type I and type II cytokine receptors lack intrinsic enzymatic activity capable of mediating signal transduction, and thus require association with tyrosine kinases for this purpose. The JAK family of kinases comprises four different members, namely JAK1, JAK2, JAK3 and TYK2, which bind to type I and type II cytokine receptors for controlling signal transduction (Murray P J, (2007). The JAK-STAT signalling pathway: input and output integration. J Immunol, 178: 2623). Each of the JAK kinases is selective for the receptors of certain cytokines. In this regard, JAK-deficient cell lines and mice have validated the essential role of each JAK protein in receptor signalling: JAK1 in class II cytokine receptors (IFN and IL-10 family), those sharing the gp130 chain (IL-6 family) and the common gamma chain (IL-2, IL-4, IL-7, IL-9, IL-15 and IL-21) (Rodig et al. (1998). Disruption of the JAK1 gene demonstrates obligatory and nonredundant roles of the Jaks in cytokine-induced biological response. Cell, 93:373; Guschin et al. (1995). A major role for the protein tyrosine kinase JAK1 in the JAK/STAT signal transduction pathway in response to interleukin-6. EMBO J. 14: 1421; Briscoe et al. (1996). Kinase-negative mutants of JAK1 can sustain intereferon-gamma-inducible gene expression but not an antiviral state. EMBO J. 15:799); JAK2 in hematopoietic factors (Epo, Tpo, GM-CSF, IL-3, IL-5) and type II IFNs (Parganas et al., (1998). JAK2 is essential for signalling through a variety of cytokine receptors. Cell, 93:385); JAK3 in receptors sharing the common gamma chain (IL-2 family) (Park et al., (1995). Developmental defects of lymphoid cells in JAK3 kinase-deficient mice. Immunity, 3:771; Thomis et al., (1995). Defects in B lymphocyte maturation and T lymphocyte activation in mice lacking JAK3. Science, 270:794; Russell et al., (1995). Mutation of JAK3 in a partient with SCID: Essential role of JAK3 in lymphoid development. Science, 270:797); and Tyk2 in the receptors of IL-12, IL-23, IL-13 and type I IFNs (Karaghiosoff et al., (2000). Partial impairment of cytokine responses in Tyk2-deficient mice. Immunity, 13:549; Shimoda et al., (2000). Tyk2 plays a restricted role in IFNg signaling, although it is required for IL-12-mediated T cell function. Immunity, 13:561; Minegishi et al., (2006). Human Tyrosine kinase 2 deficiency reveals its requisite roles in multiple cytokine signals involved in innate and acquired immunity. Immunity, 25:745).

Receptor stimulation leads sequentially to JAK activation by phosphorylation, receptor phosphorylation, STAT protein recruitment and STAT activation and dimerization. The STAT dimer then functions as a transcription factor, translocating to the nucleus and activating the transcription of multiple response genes. There are seven STAT proteins identified: STAT1, STAT2, STAT3, STAT4, STAT5a, STAT5b and STATE. Each particular cytokine receptor associates preferentially with a particular STAT protein. Some associations are independent of cell type (ex: IFNg-STAT1) while others may be cell type dependent (Murray P J, (2007). The JAK-STAT signaling pathway: input and output integration. J Immunol, 178: 2623).

The phenotype of deficient mice has provided insights on the function of each JAK and the cytokine receptors signaling through them. JAK3 associates exclusively with the common gamma chain of the receptors for IL-2, IL-4, IL-7, IL-9, IL-15 and IL-21 cytokines. By virtue of this exclusive association, JAK3 knock out mice and common gamma chain deficient mice have an identical phenotype (Thomis et al., (1995). Defects in B lymphocyte maturation and T lymphocyte activation in mice lacking JAK3. Science, 270:794; DiSanto et al., (1995). Lymphoid development in mice with a targeted deletion of the interleukin 2 receptor gamma chain. PNAS, 92:377). Moreover, this phenotype is shared to a great extent with SCID patients that hold mutations/defects in the common gamma chain or JAK3 genes (O'Shea et al., (2004). JAK3 and the pathogenesis of severe combined immunodeficiency. Mol Immunol, 41: 727). JAK3-deficient mice are viable but display abnormal lymphopoiesis which leads to a reduced thymus size (10-100 fold smaller than wild type). JAK3-deficient peripheral T cells are unresponsive and have an activated/memory cell phenotype (Baird et al, (1998). T cell development and activation in JAK3-deficient mice. J. Leuk. Biol. 63: 669). The thymic defect in these mice strongly resembles that seen in IL-7 and IL-7 receptor knockout mice, suggesting that the absence of IL-7 signaling accounts for this defect in JAK3−/− mice (von Freeden-Jeffry et al., (1995). Lymphopenia in Interleukin (IL)-7 Gene-deleted Mice Identifies IL-7 as a non-redundant Cytokine. J Exp Med, 181:1519; Peschon et al, (1994). Early lymphocyte expansion is severely impaired in interleukin 7 receptor-deficient mice. J Exp Med, 180: 1955). These mice, like SCID humans, have no NK cells, probably due to the absence of IL-15 signaling, a survival factor for these cells. JAK3 knockout mice, unlike SCID patients, show deficient B cell lymphopoiesis while in human patients, B cells are present in circulation but are not responsive leading to hypoglobulinemia (O'Shea et al., (2004). JAK3 and the pathogenesis of severe combined immunodeficiency. Mol Immunol, 41: 727). This is explained by species-specific differences in IL-7 function in B and T cell development in mice and humans. On the other hand, Grossman et al. (1999. Dysregulated myelopoiesis in mice lacking JAK3. Blood, 94:932:939) have shown that the loss of JAK3 in the T-cell compartment drives the expansion of the myeloid lineages leading to dysregulated myelopoiesis.

JAK2-deficient mice are embrionically lethal, due to the absence of definitive erythropoiesis. Myeloid progenitors fail to respond to Epo, Tpo, IL-3 or GM-CSF, while G-CSF and IL-6 signaling are not affected. JAK2 is not required for the generation, amplification or functional differentiation of lymphoid progenitors (Parganas et al., (1998). JAK2 is essential for signaling through a variety of cytokine receptors. Cell, 93:385).

JAK1-deficient mice die perinatally due to a nursing defect. JAK1 binds exclusively to the gp130 chain shared by the IL-6 cytokine family (i.e. LIF, CNTF, OSM, CT-1) and along with JAK3, is an essential component of the receptors sharing the common gamma chain, by binding to the non-shared receptor subunit. In this regard, JAK1-deficient mice show similar hematopoiesis defects as JAK3-deficient mice. In addition, they show defective responses to neurotrophic factors and to all interferons (class II cytokine receptors) (Rodig et al, (1998). Disruption of the JAK1 gene demonstrates obligatory and non-redundant roles of the JAKs in cytokine-induced biological response. Cell, 93:373).

Finally, Tyk2-deficient mice show an impaired response to IL-12 and IL-23 and only partially impaired to IFN-alpha (Karaghiosoff et al., (2000). Partial impairment of cytokine responses in Tyk2-deficient mice. Immunity, 13:549; Shimoda et al., (2000). Tyk2 plays a restricted role in IFNg signaling, although it is required for IL-12-mediated T cell function. Immunity, 13:561). However, human Tyk2 deficiency demonstrates that Tyk2 is involved in the signaling from IFN-α, IL-6, IL-10, IL-12 and IL-23 (Minegishi et al., (2006). Human Tyrosine kinase 2 deficiency reveals its requisite roles in multiple cytokine signals involved in innate and acquired immunity. Immunity, 25:745).

The role of JAK kinases in transducing the signal from a myriad of cytokines makes them potential targets for the treatment of diseases in which cytokines have a pathogenic role, such as inflammatory diseases, including but not limited to allergies and asthma, chronic obstructive pulmonary disease (COPD), psoriasis, autoimmune diseases such as rheumatoid arthritis, amyotrophic lateral sclerosis and multiple sclerosis, uveitis, transplant rejection, as well as in solid and hematologic malignancies such as myeloproliferative disorders, leukemia and lymphomas.

Inhibition of JAK kinases, especially JAK1 and JAK3, could give rise to potent immunosuppression which could be used therapeutically to prevent transplant rejection. In this regard, the JAK inhibitor CP-690,550 (tasocitinib) has shown efficacy in several animal models of transplantation (heretopic heart transplantation in mice, cardiac allografts implanted in the ear of mice, renal allotransplantation in cynomolgous monkeys, aorta and tracheal transplantation in rats) by prolonging the mean survival time of grafts (West K (2009). CP-690,550, a JAK3 inhibitor as an immunosuppressant for the treatment of rheumatoid arthritis, transplant rejection, psoriasis and other immune-mediated disorders. Curr. Op. Invest. Drugs 10: 491).

In rheumatoid joints, an imbalance between pro and anti-inflammatory cytokine activities favours the induction of autoimmunity, followed by chronic inflammation and tissue destruction. In this regard, the pathogenic role of IL-6 in rheumatoid arthritis (RA) has been validated clinically by the use of the anti-IL-6R antibody tocilizumab. IL-6 activates the transcription factor STAT3, through the use of JAK1 binding to the gp130 receptor chain (Heinrich et al., (2003). Principles of interleukin (IL)-6-type cytokine signaling and its regulation. Biochem J. 374: 1). Constitutive STAT3 mediates the abnormal growth and survival properties of RA synoviocytes (Ivashkiv and Hu (2003). The JAK/STAT pathway in rheumatoid arthritis: pathogenic or protective? Arth & Rheum. 48:2092). Other cytokines that have been implicated in the pathogenesis of arthritis include IL-12 and IL-23, implicated in Th1 and Th17 cell proliferation, respectively; IL-15, and GM-CSF (McInnes and Schett, (2007). Cytokines in the pathogenesis of rheumatoid arthritis. Nature Rew Immunol. 7:429). The receptors for these cytokines also utilize JAK proteins for signal transduction, making JAK inhibitors potential pleiotropic drugs in this pathology. Consequently, administration of several JAK inhibitors in animal models of murine collagen-induced arthritis and rat adjuvant-induced arthritis has shown to reduce inflammation, and tissue destruction (Milici et al., (2008). Cartilage preservation by inhibition of Janus kinase 3 in two rodent models of rheumatoid arthritis. Arth. Res. 10:R14).

Inflammatory bowel disease (IBD) encloses two major forms of intestinal inflammation: ulcerative colitis and Crohn's disease. Growing evidence has shown that multiple cytokines, including interleukins and interferons, are involved in the pathogenesis of IBD (Strober et al, (2002). The immunology of mucosal models of inflammation. Annu Rev Immunol. 20: 495). Activation of the IL-6/STAT3 cascade in lamina propia T cells has been shown to induce prolonged survival of pathogenic T cells (Atreya et al, (2000). Blockade of interleukin 6 trans signaling suppresses T-cell resistance against apoptosis in chronic intestinal inflammation: Evidence in Crohn's disease and experimental colitis in vivo. Nature Med. 6:583). Specifically, STAT3 has been shown to be constitutively active in intestinal T cells of Crohn's disease patients and a JAK inhibitor has been shown to block the constitutive activation of STAT3 in these cells (Lovato et al, (2003). Constitutive STAT3 activation in intestinal T cells from patients with Crohn's disease. J Biol Chem. 278:16777). These observations indicate that the JAK-STAT pathway plays a pathogenic role in IBD and that a JAK inhibitor could be therapeutic in this setting.

Multiple sclerosis is an autoimmune demyelinating disease characterized by the formation of plaques in the white matter. The role of cytokines in the generation of multiple sclerosis has long been known. Potential therapies include blockade of IFN-g, IL-6, IL-12 and IL-23 (Steinman L. (2008). Nuanced roles of cytokines in three major human brain disorders. J Clin Invest. 118:3557), cytokines that signal through the JAK-STAT pathways. Use of tyrphostin, a JAK inhibitor, has been shown to inhibit IL-12-induced phosphorylation of STAT3, and to reduce the incidence and severity of active and passive experimental autoimmune encephalitis (EAE) (Bright et al., (1999) Tyrphostin B42 inhibits IL-12-induced tyrosine phosphorylation and activation of Janus kinase-2 and prevents-experimental allergic encephalomyelitis. J Immunol. 162:6255). Another multikinase inhibitor, CEP701, has been shown to reduce secretion of TNF-alpha, IL-6 and IL-23 as well as the levels of phospho-STAT1, STAT3, and STATS in peripheral DCs of mice with EAE, significantly improving the clinical course of EAE in mice (Skarica et al, (2009). Signal transduction inhibition of APCs diminishes Th17 and Th1 responses in experimental autoimmune encephalomyelitis. J. Immunol. 182:4192).

Psoriasis is a skin inflammatory disease which involves a process of immune cell infiltration and activation that culminates in epithelial remodeling. The current theory behind the cause of psoriasis states the existence of a cytokine network that governs the interaction between immune and epithelial cells (Nickoloff B J. (2007). Cracking the cytokine code in psoriasis, Nat Med, 13:242). In this regard, IL-23 produced by dendritic cells is found elevated in psoriatic skin, along with IL-12. IL-23 induces the formation of Th17 cells which in turn produce IL-17 and IL-22, the last one being responsible for epidermis thickening. IL-23 and IL-22 induce the phosphorylation of STAT-3, which is found abundantly in psoriatic skin. JAK inhibitors may thus be therapeutic in this setting. In accordance, a JAK1/3 inhibitor, R348, has been found to attenuate psoriasiform skin inflammation in a spontaneous T cell-dependent mouse model of psoriasis (Chang et al., (2009). JAK3 inhibition significantly attenuates psoriasiform skin inflammation on CD18 mutant PL/J mice. J Immunol. 183:2183).

Th2 cytokine-driven diseases such as allergy and asthma could also be a target of JAK inhibitors. IL-4 promotes Th2 differentiation, regulates B-cell function and immunoglobulin class switching, regulates eotaxin production, induces expression of IgE receptor and MHC II on B cells, and stimulates mast cells. Other Th2 cytokines like IL-5 and IL-13 can also contribute to eosinophil recruitment in bronchoalveolar lavage by stimulating eotaxin production. Pharmacological inhibition of JAK has been shown to reduce the expression of IgE receptor and MHCII induced by IL-4 stimulation on B cells (Kudlacz et al., (2008). The JAK3 inhibitor CP-690,550 is a potent anti-inflammatory agent in a murine model of pulmonary eosinophilia. European J. Pharm. 582: 154). Furthermore, JAK3-deficient mice display poor eosinophil recruitment and mucus secretion to the airway lumen upon OVA challenge, as compared to wild type mice (Malaviya et al, (2000). Treatment of allergic asthma by targeting Janus kinase 3-dependent leukotriene synthesis in mast cells with 4-(3′,5′-dibromo-4′-hydroxyphenyl)amino-6,7-dimethoxyquinazoline (WHI-P97). JPET 295:912). In this regard, systemic administration of the CP-690,550 JAK inhibitor in mice has been shown to reduce the eosinophil count as well as the levels of eotaxin and IL13 in BAL in a murine model of pulmonary eosinophilia (Kudlacz et al., (2008). The JAK3 inhibitor CP-690,550 is a potent anti-inflammatory agent in a murine model of pulmonary eosinophilia. European J. Pharm. 582:154).

There is increasing evidence that cytokines play a pathogenetic role in ocular inflammatory disease such as uveitis or dry eye syndrome. Some cytokines implicated in experimental autoimmune uveitis, such as IL-2, IL-6, IL-12 and IFNg, would be amenable to JAK inhibition (Vallochi et al, (2007). The role of cytokines in the regulation of ocular autoimmune inflammation. Cytok Growth Factors Rev. 18:135). In this regard, drugs or biologicals that interfere with IL-2 signaling such as cyclosporine or anti-IL-2 receptor antibody (daclizumab) have shown efficacy in the treatment of keratoconjuctivitis sicca and refractory uveitis, respectively (Lim et al, (2006). Biologic therapies for inflammatory eye disease. Clin Exp Opht 34:365). Similarly, allergic conjunctivitis, a common allergic eye disease characterized by conjuctival congestion, mast cell activation and eosinophil infiltration, could benefit from JAK inhibition. STAT6-deficient mice, showing decreased TH2-mediated immune responses which are normally triggered by IL-4, do not develop the classical early and late phase responses, suggesting that IL-4 pathway abrogation through JAK inhibition may be therapeutic in this setting (Ozaki et al, (2005). The control of allergic conjunctivitis by suppression of cytokine signaling (SOCS)3 and SOCS5 in a murine model. J Immunol, 175:5489).

There is growing evidence of the critical role of STAT3 activity in processes involved in tumorigenesis like cell cycle dysregulation, promotion of uncontrolled growth, induction of survival factors and inhibition of apoptosis (Siddiquee et al., (2008). STAT3 as a target for inducing apoptosis in solid and haematological tumors. Cell Res. 18: 254). Antagonism of STAT3 by means of dominant-negative mutants or antisense oligonucleotides has shown to promote apoptosis of cancer cells, inhibition of angiogenesis and up-regulation of host immunocompetence. Inhibition of constitutively active STAT3 in human tumors by means of JAK inhibitors may provide a therapeutic option to the treatment of this disease. In this regard, the use of the JAK inhibitor tyrphostin has been shown to induce apoptosis of malignant cells and inhibit cell proliferation in vitro and in vivo (Meydan et al., (1996). Inhibition of acute lymphoblastic leukemia by a JAK-2 inhibitor. Nature, 379:645).

Hematological malignancies with dysregulated JAK-STAT pathways may benefit from JAK inhibition. Recent studies have implicated dysregulation of JAK2 kinase activity by chromosomal translocations and mutations within the pseudokinase domain (such as the JAK2V617F mutation) in a spectrum of myeloproliferative diseases (Ihle and Gililand, 2007), including polycythemia vera, myelofibrosis and essential thrombocythemia. In this regard, several JAK inhibitors that tackle JAK2 potently, such as TG-101209 (Pardanani et al., (2007). TG101209, a small molecular JAK2-selective inhibitor potently inhibits myeloproliferative disorder-associated JAK2V617F and MPLW515L/K mutations Leukemia. 21:1658-68), TG101348 (Wernig et al, (2008). Efficacy of TG101348, a selective JAK2 inhibitor, in treatment of a murine model of JAK2V617F-induced polycythemia vera. Cancer Cell, 13: 311), CEP701, (Hexner et al, (2008). Lestaurtinib (CEP701) is a JAK2 inhibitor that suppresses JAK2/STAT5 signaling and the proliferation of primary erythroid cells from patients with myeloproliferative disorders. Blood, 111: 5663), CP-690,550 (Manshouri et al, (2008). The JAK kinase inhibitor CP-690,550 suppresses the growth of human polycythemia vera cells carrying the JAK2V617F mutation. Cancer Sci, 99:1265), and CYT387 (Pardanani et al., (2009). CYT387, a selective JAK1/JAK2 inhibitor: invitro assessment of kinase selectivity and preclinical studies using cell lines and primary cells from polycythemia vera patients. Leukemia, 23:1441) have been proposed for treating myeloproliferative diseases on the basis of their antiproliferative activity on cells carrying the JAK2V617F mutation. Similarly, T-cell leukemia due to human T-cell leukemia virus (HTLV-1) transformation is associated with JAK3 and STAT5 constitutive activation (Migone et al, (1995). Constitutively activated JAK-STAT pathway in T cells transformed with HTLV-I. Science, 269: 79) and JAK inhibitors may be therapeutic in this setting (Tomita et al, (2006). Inhibition of constitutively active JAK-STAT pathway suppresses cell growth of human T-cell leukemia virus type I-infected T cell lines and primary adult T-cell leukemia cells. Retrovirology, 3:22). JAK1-activating mutations have also been identified in adult acute lymphoblastic leukemia of T cell origin (Flex et al, (2008). Somatically acquired JAK1 mutations in adult acute lymphoblastic leukemia. J. Exp. Med. 205:751-8) pointing to this kinase as a target for the development of novel antileukemic drugs.

Conditions in which targeting of the JAK pathway or modulation of the JAK kinases, particularly JAK1, JAK2 and JAK3 kinases, are contemplated to be therapeutically useful for the treatment or prevention of diseases include: neoplastic diseases (e.g. leukemia, lymphomas, solid tumors); transplant rejection, bone marrow transplant applications (e.g., graft-versus-host disease); autoimmune diseases (e.g. diabetes, multiple sclerosis, rheumatoid arthritis, inflammatory bowel disease); respiratory inflammation diseases (e.g. asthma, chronic obstructive pulmonary disease), inflammation-linked ocular diseases or allergic eye diseases (e.g. dry eye, glaucoma, uveitis, diabetic retinopathy, allergic conjunctivitis or age-related macular degeneration) and skin inflammatory diseases (e.g., atopic dermatitis or psoriasis).

In view of the numerous conditions that are contemplated to benefit by treatment involving modulation of the JAK pathway or of the JAK kinases it is immediately apparent that new compounds that modulate JAK pathways and use of these compounds should provide substantial therapeutic benefits to a wide variety of patients.

Provided herein are novel imidazopyridine compounds for use in the treatment of conditions in which targeting of the JAK pathway or inhibition of JAK kinases can be therapeutically useful.

The compounds described in the present invention are simultaneously potent JAK1, JAK2 and JAK3 inhibitors, i.e. pan-JAK inhibitors. This property makes them useful for the treatment or prevention of pathological conditions or diseases such as myeloproliferative disorders (such as polycythemia vera, essential thrombocythemia or mielofibrosis), leukemia, lymphomas and solid tumors; bone marrow and organ transplant rejection; or immune-mediated diseases such as autoimmune and inflammation diseases, including rheumatoid arthritis, multiple sclerosis, inflammatory bowel disease (such as ulcerative colitis or Crohn's disease), inflammation-linked ocular diseases or allergic eye diseases (such as dry eye, uveitis, or allergic conjunctivitis), allergic rhinitis, asthma, chronic obstructive pulmonary disease (COPD), and skin inflammatory diseases (such as atopic dermatitis or psoriasis).

It has now been found that certain imidazopyridine derivatives are novel and potent JAK inhibitors and can therefore be used in the treatment or prevention of these diseases.

Thus the present invention is directed to compounds which are imidazopyridine derivatives of formula (I), or a pharmaceutically acceptable salt, or solvate, or N-oxide or stereoisomer thereof, for use in the treatment of a pathological condition or disease susceptible to amelioration by inhibition of Janus Kinases (JAK):

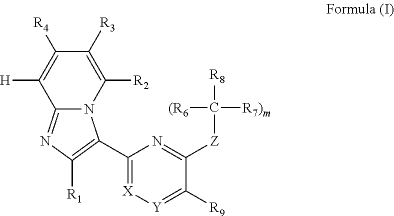

wherein,
m is 0 or an integer from 1 to 3;
Z is an oxygen atom or a group NR₅;
X and Y independently represent a nitrogen atom or a —CR₉ group, wherein at least one of X and Y represents a nitrogen atom;
R₁, R₂, R₃, R₄ and R₉ each independently represent a hydrogen atom, a halogen atom, a cyano group, a linear or branched C₁-C₆ alkyl group, a C₂-C₄ alkenyl group, a C₂-C₄ alkynyl group, a C₁-C₄ haloalkyl group, a C₁-C₄ hydroxyalkyl group, a C₃-C₁₀ cycloalkyl group, a C₃-C₁₀ cycloalkenyl group, a (C₁-C₄ alkyl)(C₃-C₇) cycloalkyl group, a monocyclic or polycyclic C₅-C₁₄ aryl group, a 5- to 14-membered heteroaryl group containing at least one heteroatom selected from O, S and N, a 5- to 14-membered heterocyclyl group containing at least one heteroatom selected from O, S and N, a bicyclyl group containing a monocyclic C₅-C₉ aryl or heteroaryl group bonded directly to a 5- to 9-membered cycloalkyl or heterocyclyl group, said heteroaryl or heterocyclyl group containing at least one heteroatom selected from O, S and N, an aza-bicycloalkyl group having up to 12 carbon atoms or an aza-bicycloalkenyl group having up to 12 carbon atoms,
   wherein the alkenyl, alkynyl, haloalkyl, hydroxyalkyl, cycloalkyl, cycloalkenyl, aryl, heteroaryl, heterocyclyl, bicyclyl, aza-bicycloalkyl and aza-bicycloalkenyl groups are unsubstituted or substituted by one or more substituents selected from substituents Ra, and the alkyl groups are unsubstituted or substituted with one or more substituents selected from Rb;
or R₁, R₂, R₃, R₄ and R₉ independently represent a —SR₁₃ group, a —SOR₁₃ group, a —S(O)₂R₁₃ group, a —S(O)₂NR₁₃R₁₄ group, a —NR₁₃S(O)₂R₁₄ group, a —NR₁₃S(O)₂NR₁₄ group, a —OR₁₃ group, a —C(O)OR₁₃ group, a —O—C(O)R₁₃ group, a —C(O)—(CH₂)ₙ—R₁₃ group, a —NR₁₃R₁₄ group, a —C(O)—(CH₂)ₙ—NR₁₃R₁₄ group, a —NR₁₃C(O)—(CH₂)ₙ—R₁₄ group or a —NR₁₃C(O)—(CH₂)ₙ—NR₁₄R₁₅ group, wherein each n is 0, 1 or 2;
or in the case when two adjacent —CR₉ groups are present, two adjacent —CR₉ groups and the carbon atoms to which they are bonded optionally form a C₅-C₁₂ aryl group or a 4- to 12-membered heteroaryl, cycloalkyl or heterocyclyl group, said heteroaryl and heterocyclyl groups containing at least one heteroatom selected from O, S and N, the aryl, heteroaryl, cycloalkyl and heterocyclyl groups being unsubstituted or substituted by one or more substituents selected from a halogen atom, a linear or branched C₁-C₆ alkyl group, a monocyclic or polycyclic C₅-C₁₄ aryl group, a 5- to 14-membered heteroaryl group containing at least one heteroatom selected from O, S and N, or a 5- to 14-membered heterocyclyl group containing at least one heteroatom selected from O, S and N, wherein the alkyl, the aryl, the heteroaryl and the heterocyclyl substituents are unsubstituted or substituted by one or more substituents selected from a halogen atom, a hydroxy group, a cyano group, a linear or branched C₁-C₆ alkyl group or a C₁-C₄ haloalkyl group;
R₅ represents a hydrogen atom, a linear or branched C₁-C₆ alkyl group optionally substituted by one or more substituents selected from a hydroxy group, a cyano group, a C₁-C₄ haloalkyl group, a C₁-C₄ hydroxyalkyl group, a C₃-C₁₀ cycloalkyl group, a phenyl group or a 6 membered, saturated N-containing heterocyclyl ring, or R₅ represents a —S(O)₂R₁₀ group, a —S(O)₂NR₁₀R₁₁ group, a —C(O)OR₁₀ group, a —C(O)—(CH₂)ₙ—R₁₀ group, or a —C(O)—(CH₂)ₙ—NR₁₀R₁₁ group;
R₆ and R₇ each independently represents a hydrogen atom or a linear or branched C₁-C₆ alkyl group optionally substituted by one or more substituents selected from a hydroxy group, a cyano group, a C₁-C₄ haloalkyl group, a C₁-C₄ hydroxyalkyl group, a C₁-C₄ alkoxy group, a C₁-C₄ alkoxycarbonyl group, a C₃-C₇ cycloalkyl group, a phenyl group or a 6 membered, saturated N-containing heterocyclyl ring;
R₈ represents a hydrogen atom, a halogen atom, a cyano group, a linear or branched C₁-C₆ alkyl group, a C₂-C₄ alkenyl group, a C₂-C₄ alkynyl group, a C₁-C₄ haloalkyl group, a C₁-C₄ hydroxyalkyl group, a —(C₁-C₆ alkyl)-(C₁-C₄ alkoxy) group, a C₃-C₁₀ cycloalkyl group, a C₃-C₁₀ cycloalkenyl group, a monocyclic or polycyclic C₅-C₁₄ aryl group, a 5- to 14-membered heteroaryl group containing at least one heteroatom selected from O, S and N, a 5- to 14-membered heterocyclyl group containing at least one heteroatom selected from O, S and N, a C₃-C₇ heterocycloalkyl ketone group containing 1, 2 or 3 nitrogen atoms, a bicyclyl group containing a monocyclic C₅-C₉ aryl or heteroaryl group bonded directly to a 5- to 9-membered cycloalkyl or heterocyclyl group, said heteroaryl or heterocyclyl group containing at least one heteroatom selected from O, S and N, an aza-bicycloalkyl group having up to 12 carbon atoms or a aza-bicycloalkenyl group having up to 12 carbon atoms,
   wherein the alkenyl, alkynyl, haloalkyl, hydroxyalkyl, cycloalkyl, cycloalkenyl, aryl, heteroaryl, heterocyclyl, heterocycloalkyl ketone, bicyclyl, aza-bicycloalkyl and aza-bicycloalkenyl groups are unsubstituted or substituted by one or more substituents selected from Ra, —(C₁-C₄ alkyl)-CN groups, or —(C₁-C₄ alkyl)-C(O)NR′R″ groups wherein R′ and R″ are the same or different and are selected from hydrogen atoms and linear or branched C₁-C₄ alkyl groups; and the alkyl groups are unsubstituted or substituted by one or more substituents selected from Rb;
or R₅ represents a —SR₁₃ group, a —SOR₁₃ group, a —S(O)₂R₁₃ group, a —S(O)₂NR₁₃R₁₄ group, a —NR₁₃S(O)₂R₁₄ group, a —NR₁₃S(O)₂NR₁₄ group, a —OR₁₃ group, a —C(O)OR₁₃ group, a —O—C(O)R₁₃ group, a —C(O)—(CH₂)ₙ—R₁₃ group, a —NR₁₃R₁₄ group, a —C(O)—(CH₂)ₙ—NR₁₃R₁₄ group, a —NR₁₃C(O)—(CH₂)ₙ—R₁₄ group, or a —NR₁₃C(O)—(CH₂)ₙ—NR₁₄R₁₅ group, wherein n is 0, 1 or 2,
or R₈ together with R₅ and the nitrogen atom to which R₅ is bonded form a 4- to 10-membered, saturated heterocyclyl group, which contains, as heteroatoms, one or two nitrogen atoms and which is substituted by a linear or branched C₁-C₆ alkyl group, a monocyclic or polycyclic C₅-C₁₄ aryl group, a 5- to 14-membered heteroaryl group containing at least one heteroatom selected from O, S and N, a 5- to 14-membered heterocyclyl group containing at least one heteroatom selected from O, S and N, a —SOR₁₀ group, a —C(O)—(CH₂)ₙ—R₁₀ group, or a —C(O)—(CH₂)ₙ—NR₁₀R₁₁ group, wherein each n is 0, 1 or 2,
   wherein the alkyl, aryl, heteroaryl and heterocyclyl groups are unsubstituted or substituted by one or more substituents selected from a halogen atom, a hydroxy group, a cyano group, a linear or branched C₁-C₆ alkyl group or a C₁-C₄ haloalkyl group, and wherein the alkyl groups are unsubstituted or substituted by one or more substituents selected from a halogen atom, a hydroxy group, a cyano group or a C₁-C₄ haloalkyl group;
provided that when m is zero, R₈ is other than a —SR₁₃ group, a —SOR₁₃ group, a —S(O)₂R₁₃ group, a —S(O)₂NR₁₃R₁₄ group, a —NR₁₃S(O)₂R₁₄ group, a —NR₁₃S(O)₂NR₁₄ group, a —OR₁₃ group, a —O—C(O)R₁₃ group, a —NR₁₃R₁₄ group, a —NR₁₃C(O)—(CH₂)ₙ—R₁₄ group, or a —NR₁₃C(O)—(CH₂)ₙ—NR₁₄R₁₅ group,
   wherein
Ra is a halogen atom, a cyano group, a hydroxy group, a linear or branched C₁-C₆ alkyl group, which alkyl group is unsubstituted or substituted by one or more cyano groups, a C₁-C₄ haloalkyl group, a C₁-C₄ hydroxyalkyl group, a C₃-C₇ cycloalkyl or a C₃-C₇ cycloalkenyl group, a monocyclic or polycyclic C₅-C₁₄ aryl group, which aryl group is unsubstituted or substituted by one or more substituents selected from a halogen atom or a cyano group, a 5- to 14-membered heteroaryl group containing at least one heteroatom selected from O, S and N, which heteroaryl group is unsubstituted or substituted by one or more substituents selected from a halogen atom or a cyano group, a 5- to 14-membered heterocyclyl group containing at least one heteroatom selected from O, S and N, a —SR₁₀ group, a —SOR₁₀ group, a —S(O)₂R₁₀ group, a —S(O)₂NR₁₀R₁₁ group, a —NR₁₀S(O)₂R₁₁ group, a —NR₁₀S(O)₂NR₁₁ group, a —OR₁₀ group, a —C(O)OR₁₀ group, a —C(O)—CH₂—OR₁₀ group, a —C(O)—(CH₂)ₙ—O—(CH₂)ₚR₁₀ group, a —C(O)—(CH₂)ₙ—O—(CH₂)ₚO—R₁₀ group, a —O—C(O)R₁₀ group, a —C(O)—(CH₂)ₙ—R₁₀ group, a —NR₁₀R₁₁ group, a —C(O)—(CH₂)ₙ—NR₁₀R₁₁ group, a —R₁₀C(O)—(CH₂)ₙ—NR₁₁R₁₂ group, a —C(C₁-C₄ alkyl)₂-C(O)—(CH₂)ₙ—NR₁₀R₁₁ group, a —NR₁₀C(O)—(CH₂)ₙ—R₁₁ group or a —NR₁₀C(O)—(CH₂)ₙ—NR₁₁R₁₂ group, wherein each n is 0 or an integer from 1 to 5, and wherein p is an integer from 1 to 3;
Rb is a cyano group, a hydroxy group, a linear or branched C₁-C₆ alkyl group, a C₁-C₄ haloalkyl group, a C₁-C₄ hydroxyalkyl group, a C₃-C₇ cycloalkyl or a C₃-C₇ cycloalkenyl group, a monocyclic or polycyclic C₅-C₁₄ aryl group, which aryl group is unsubstituted or substituted by one or more substituents selected from a halogen atom or a cyano group, a 5- to 14-membered heteroaryl group containing at least one heteroatom selected from O, S and N, which heteroaryl group is unsubstituted or substituted by one or more substituents selected from a halogen atom or a cyano group, a 5- to 14-membered heterocyclyl group containing at least one heteroatom selected from O, S and N, a —SR₁₀ group, a —SOR₁₀ group, a —S(O)₂R₁₀ group, a —S(O)₂NR₁₀R₁₁ group, a —NR₁₀S(O)₂R₁₁ group, a —NR₁₀S(O)₂NR₁₁ group, a —OR₁₀ group, a —C(O)OR₁₀ group, a —C(O)—CH₂—OR₁₀ group, a —C(O)—(CH₂)ₙ—O—(CH₂)ₚR₁₀ group, a —C(O)—(CH₂)ₙ—O—(CH₂)ₚO—R₁₀ group, a —O—C(O)R₁₀ group, a —C(O)—(CH₂)ₙ—R₁₀ group, a —NR₁₀R₁₁ group, a —C(O)—(CH₂)ₙ—NR₁₀R₁₁ group, a —R₁₀C(O)—(CH₂)ₙ—NR₁₁R₁₂ group, a —C(C₁-C₄ alkyl)₂-C(O)—(CH₂)ₙ—NR₁₀R₁₁ group, a —NR₁₀C(O)—(CH₂)ₙ—R₁₁ group or a —NR₁₀C(O)—(CH₂)ₙ—NR₁₁R₁₂ group, wherein each n is 0 or an integer from 1 to 5, and wherein p is an integer from 1 to 3;
R₁₀, R₁₁ and R₁₂ each independently represents a hydrogen atom, a cyano group, a linear or branched C₁-C₆ alkyl group, a C₁-C₄ haloalkyl group, a C₁-C₆ hydroxyalkyl group, a C₁-C₄ alkoxycarbonyl group, a C₃-C₇ cycloalkyl group, a phenyl group, a —(C₁-C₄ hydroxyalkyl)-(phenyl) group, a —C(O)-(phenyl) group, a 5- to 6-membered monocyclic heteroaryl group containing 1, 2, 3 or 4 heteroatoms selected from N, O and S, a 5- to 6-membered, heterocyclyl group containing 1, 2 or 3 heteroatoms selected from oxygen and nitrogen atoms, a C₃-C₇ heterocycloalkyl ketone group containing 1, 2 or 3 nitrogen atoms, a bicyclyl group containing a monocyclic C₅-C₆ aryl or heteroaryl group bonded directly to a 5- to 6-membered cycloalkyl or heterocyclyl group which heteroaryl or heterocyclyl group contains 1, 2 or 3 heteroatoms selected from oxygen and nitrogen atoms, the haloalkyl, hydroxyalkyl, alkoxycarbonyl, cycloalkyl, phenyl, hydroxyalkyl-phenyl, heteroaryl, heterocyclyl, heterocycloalkyl ketone and bicyclyl groups being unsubstituted or substituted by one or more substituents selected from substituents Rc, and the alkyl groups being unsubstituted or substituted by one or more substituents selected from substituents Rd;
Rc is a halogen atom, a hydroxy group, a cyano group, a linear or branched C₁-C₆ alkyl group, a C₁-C₄ haloalkyl group, a C₁-C₄ alkoxy group, a C₁-C₄ hydroxyalkyl group, a C₃-C₇ cycloalkyl group, a phenyl group, a 5- to 6-membered monocyclic heteroaryl group containing 1, 2 or 3 nitrogen atoms, a 5- to 6-membered heterocyclyl group containing 1, 2 or 3 nitrogen atoms, or a C₃-C₇ heterocycloalkyl ketone group containing 1, 2 or 3 nitrogen atoms, said phenyl group being unsubstituted or substituted with one or more halogen atoms, and said heteroaryl, heterocyclyl and heterocycloalkyl ketone groups being unsubstituted or substituted by one or more linear or branched C₁-C₃ alkyl groups;
Rd is a cyano group, a C₁-C₄ haloalkyl group, a C₁-C₄ alkoxy group, a C₁-C₄ hydroxyalkyl group, a C₃-C₇ cycloalkyl group, a phenyl group, a 5- to 6-membered monocyclic heteroaryl group containing 1, 2 or 3 nitrogen atoms, a 5- to 6-membered heterocyclyl group containing 1, 2 or 3 nitrogen atoms, or a C₃-C₇ heterocycloalkyl ketone group containing 1, 2 or 3 nitrogen atoms, said phenyl group being unsubstituted or substituted with one or more halogen atoms, and said heteroaryl, heterocyclyl and heterocycloalkyl ketone groups being unsubstituted or substituted by one or more linear or branched C₁-C₃ alkyl groups;
R₁₃, R₁₄, and R₁₅ each independently represents a hydrogen atom, a cyano group, a linear or branched C₁-C₆ alkyl group, a C₁-C₄ haloalkyl group, a C₁-C₄ hydroxyalkyl group, a —(C₁-C₆ alkyl)-(C₁-C₄ alkoxy) group, a —(C₁-C₆ alkyl)-(5- to 7-membered heterocyclyl group containing at least one heteroatom selected from O, S and N) group, a C₁-C₄ alkoxycarbonyl group, a C₃-C₇ cycloalkyl group, a monocyclic or polycyclic C₅-C₁₄ aryl group, a 5- to 14-membered heteroaryl group containing at least one heteroatom selected from O, S and N, or a 5- to 14-membered heterocyclyl group containing at least one heteroatom selected from O, S and N, wherein the haloalkyl, hydroxyalkyl, alkoxycarbonyl, cycloalkyl, aryl, heteroaryl and heterocyclyl groups are unsubstituted or substituted by one or more substituents selected from the substituents Ra, and the alkyl groups are optionally substituted by one or more substitutents selected from Rb.

The invention further provides new imidazopyridine derivatives of formula (I), or a pharmaceutically acceptable salt, or solvate, or N-oxide, or stereoisomer or deuterated derivative thereof, wherein m, X, Y, Z and R₁ to R₉ are as defined above, provided that when Y represents a nitrogen atom, X represents a —CR₉ group, and R₈ represents a 5- to 6-membered heterocyclyl group containing one nitrogen atom, which nitrogen atom is not bonded to the —Z—(CR₆R₇)ₘ— moiety, said nitrogen atom is substituted by a substituent other than a tert-butoxycarbonyl group or a benzyloxycarbonyl group.

The invention further provides synthetic processes and intermediates described herein, which are useful for preparing said compounds.

The invention also provides a pharmaceutical composition comprising the compounds of the invention and a pharmaceutically-acceptable diluent or carrier.

The pathological condition or disease susceptible to amelioration by inhibition of Janus Kinases (JAK) is, in particular, selected from myeloproliferative disorders, leukemia, lymphoid malignancies and solid tumors; bone marrow and organ transplant rejection; immune-mediated diseases and inflammatory diseases, for example from myeloproliferative disorders, leukemia, lymphoid malignancies and solid tumors; bone marrow and organ transplant rejection and immune-mediated diseases. More particularly the pathological condition or disease is selected from rheumatoid arthritis, multiple sclerosis, inflammatory bowel disease, dry eye, uveitis, allergic conjunctivitis, allergic rhinitis, asthma, chronic obstructive pulmonary disease (COPD), atopic dermatitis and psoriasis.

In one aspect, the compounds of formula (I) may be used in the treatment of myeloproliferative disorders, leukemia, lymphoid malignancies and solid tumors. In this aspect, the treatment is typically effected by inhibition of Janus Kinases in the subject. In another aspect, the compounds of formula (I) may be used in the treatment of bone marrow and organ transplant rejection; immune-mediated diseases and inflammatory diseases, for example from bone marrow and organ transplant rejection; and immune-mediated diseases, e.g. bone marrow and organ transplant rejection.

The invention also provides a imidazopyridine derivative formula (I) as defined herein, or a pharmaceutically acceptable salt, or solvate, or N-oxide, or stereoisomer or deuterated derivative thereof for use in inhibiting Janus Kinases. In particular, the invention provides a imidazopyridine derivative of formula (I) as defined herein, or a pharmaceutically acceptable salt, or solvate, or N-oxide, or stereoisomer or deuterated derivative thereof for treating a pathological condition or disease as described above, wherein the treatment is by inhibition of Janus Kinases.

The invention also provides a method of treatment of a pathological condition or disease susceptible to amelioration by inhibition of Janus Kinases (JAK), in particular wherein the pathological condition or disease is selected from myeloproliferative disorders, leukemia, lymphoid malignancies and solid tumors; bone marrow and organ transplant rejection; immune-mediated diseases and inflammatory diseases, for example from myeloproliferative disorders, leukemia, lymphoid malignancies and solid tumors; bone marrow and organ transplant rejection; and immune-mediated diseases, more particularly wherein the pathological condition or disease is selected from rheumatoid arthritis, multiple sclerosis, inflammatory bowel disease, dry eye, uveitis, allergic conjunctivitis, allergic rhinitis, asthma, chronic obstructive pulmonary disease (COPD), atopic dermatitis and psoriasis; comprising administering a therapeutically effective amount of the compounds defined herein or a pharmaceutical composition comprising a compound as defined herein in association with a pharmaceutically acceptable diluent or carrier to a subject in need of such treatment. In particular, the treatment is effected by inhibition of Janus Kinases in the subject.

The invention also provides a method of inhibiting Janus Kinases in a subject in need thereof, which comprises administering to said subject a therapeutically effective amount of a compound as defined herein, or a pharmaceutical composition comprising a compound as defined herein in association with a pharmaceutically acceptable diluent or carrier to a subject in need of such treatment.

The invention also provides a combination product comprising (i) a compound as described herein; and (ii) one or more additional active substances which are known to be useful in the treatment of myeloproliferative disorders (such as polycythemia vera, essential thrombocythemia or mielofibrosis), leukemia, lymphoid malignancies and solid tumors; bone marrow and organ transplant rejection; immune-mediated diseases and inflammatory diseases, for example from myeloproliferative disorders, leukemia, lymphoid malignancies and solid tumors; bone marrow and organ transplant rejection; and immune-mediated diseases, more particularly wherein the pathological condition or disease is selected from rheumatoid arthritis, multiple sclerosis, inflammatory bowel disease (such as ulcerative colitis or Crohn's disease), dry eye, uveitis, allergic conjunctivitis, allergic rhinitis, asthma, chronic obstructive pulmonary disease (COPD), atopic dermatitis and psoriasis.

As used herein the term C₁-C₆ alkyl embraces optionally substituted, linear or branched radicals having 1 to 6 carbon atoms, preferably 1 to 4 carbon atoms. Examples include methyl, ethyl, n-propyl, i-propyl, n-butyl, sec-butyl, t-butyl, n-pentyl, 1-methylbutyl, 2-methylbutyl, isopentyl, 1-ethylpropyl, 1,1-dimethylpropyl, 1,2-dimethylpropyl, n-hexyl, 1-ethyl butyl, 2-ethylbutyl-1,1-dimethylbutyl, 1,2-dimethyl butyl, 1,3-di methylbutyl, 2,2-dimethylbutyl, 2,3-dimethylbutyl, 2-methylpentyl, 3-methylpentyl and iso-hexyl radicals.

As used herein, the term C₂-C₄ alkenyl embraces optionally substituted, linear or branched, mono or polyunsaturated radicals having 2 to 4 carbon atoms. Examples include vinyl, allyl, 1-propenyl, isopropenyl, 1-butenyl, 2-butenyl and 3-butenyl radicals.

As used herein, the term C₂-C₄ alkynyl embraces optionally substituted, linear or branched, mono or polyunsaturated radicals having 2 to 4 carbon atoms. Examples include 1-propynyl, 2-propynyl, 1-butynyl, 2-butynyl and 3-butynyl radicals.

When it is mentioned that alkyl, alkenyl or alkynyl radicals may be optionally substituted it is meant to include linear or branched alkyl, alkenyl or alkynyl radicals as defined above, which may be unsubstituted or substituted in any position by one or more substituents, for example by 1, 2 or 3 substituents. When two or more substituents are present, each substituent may be the same or different.

A said optionally substituted alkenyl group is typically unsubstituted or substituted with 1, 2 or 3 substituents which may be the same or different. Typically, substituents on an alkenyl group are themselves unsubstituted. Preferred substituents on the alkenyl groups are halogen atoms and hydroxy groups, and are more preferably halogen atoms.

A said optionally substituted alkynyl group is typically unsubstituted or substituted with 1, 2 or 3 substituents which may be the same or different. Typically, substituents on an alkynyl group are themselves unsubstituted. Preferred substituents on the alkynyl groups are halogen atoms and hydroxy groups, and are more preferably halogen atoms

As used herein, the term C₁-C₄ haloalkyl group is an alkyl group, for example a C₁₄ or C₁₂ alkyl group, which is bonded to one or more, preferably 1, 2 or 3 halogen atoms. Preferably, said haloakyl group is chosen from —CCl₃ and —CF₃.

As used herein, the term C₁-C₆ hydroxyalkyl embraces linear or branched alkyl radicals having 1 to 6 carbon atoms, preferably 1 to 4 carbon atoms, one or more of which may be substituted with one or more, preferably 1 or 2, more preferably 1 hydroxyl radicals. Examples of such radicals include hydroxymethyl, hydroxyethyl, hydroxypropyl, and hydroxybutyl.

As used herein, the term C₁-C₄ alkoxy (or alkyloxy) embraces optionally substituted, linear or branched oxy-containing radicals each having alkyl portions of 1 to 4 carbon atoms. An alkoxy group is typically unsubstituted or substituted with 1, 2 or 3 substituents which may be the same or different. Typically, the substituents on an alkoxy group are themselves unsubstituted. Preferred alkoxy radicals include methoxy, ethoxy, n-propoxy, i-propoxy, n-butoxy, sec-butoxy, t-butoxy, trifluoromethoxy, difluoromethoxy, hydroxymethoxy, 2-hydroxyethoxy and 2-hydroxypropoxy.

As used herein, a C₁-C₄ alkoxycarbonyl group is typically a said C₁-C₄ alkoxy group bonded to a carbonyl group.

As used herein, the term C₃-C₁₀ cycloalkyl embraces saturated monocyclic or polycyclic carbocyclic radicals having from 3 to 10 carbon atoms, preferably from 3 to 7 carbon atoms. A C₃-C₁₀ cycloalkyl radical is typically unsubstituted or substituted with 1, 2 or 3 substituents which may be the same or different. When a C₃-C₁₀ cycloalkyl radical carries 2 or more substituents, the substituents may be the same or different. Typically the substituents on a C₃-C₁₀ cycloalkyl group are themselves unsubstituted. Polycyclic cycloalkyl radicals contains two or more fused cycloalkyl groups, preferably two cycloalkyl groups. Typically, polycyclic cycloalkyl radicals are selected from decahydronaphthyl (decalyl), bicyclo[2.2.2]octyl, adamantly, camphyl or bornyl groups. Examples of monocyclic cyclocalkyl groups include cyclopropyl, cyclobutyl, cyclopentyl, cyclohexyl, cycloheptyl, cyclooctyl, cyclononyl and cyclodecyl.

As used herein, the term C₃-C₁₀ cycloalkenyl embraces partially unsaturated carbocyclic radicals having from 3 to 10 carbon atoms, preferably from 3 to 7 carbon atoms. A C₃-C₁₀ cycloalkenyl radical is typically unsubstituted or substituted with 1, 2 or 3 substituents which may be the same or different. When a C₃-C₁₀ cycloalkenyl radical carries 2 or more substituents, the substituents may be the same or different. Typically, the substituents on a cycloalkenyl group are themselves unsubstituted.

Examples include cyclobutenyl, cyclopentenyl, cyclohexenyl, cycloheptenyl, cyclooctenyl, cyclononenyl and cyclodecenyl.

As used herein, the term C₅-C₁₄ aryl radical embraces typically a C₅-C₁₄, preferably C₆-C₁₄, more preferably C₆-C₁₀ monocyclic or polycyclic aryl radical such as phenyl, naphthyl, anthranyl and phenanthryl. Phenyl is preferred. A said optionally substituted C₅-C₁₄ aryl radical is typically unsubstituted or substituted with 1, 2 or 3 substituents which may be the same or different. When a C₅₋₁₀ aryl radical carries 2 or more substituents, the substituents may be the same or different. Unless otherwise specified, the substituents on a C₅-C₁₄ aryl group are typically themselves unsubstituted.

As used herein, the term 5- to 14-membered heteroaryl radical embraces typically a 5- to 14-membered ring system, preferably a 5- to 10-membered ring system, more preferably a 5- to 6-membered ring system, comprising at least one heteroaromatic ring and containing at least one heteroatom selected from O, S and N. A 5- to 14-membered heteroaryl radical may be a single ring or two or more fused rings wherein at least one ring contains a heteroatom.

A said optionally substituted 5- to 14-membered heteroaryl radical is typically unsubstituted or substituted with 1, 2 or 3 substituents which may be the same or different. When a 5- to 14-membered heteroaryl radical carries 2 or more substituents, the substituents may be the same or different. Unless otherwise specified, the substituents on a 5- to 14-membered heteroaryl radical are typically themselves unsubstituted.

Examples include pyridyl, pyrazinyl, pyrimidinyl, pyridazinyl, furyl, benzofuranyl, oxadiazolyl, oxazolyl, isoxazolyl, benzoxazolyl, imidazolyl, benzimidazolyl, thiazolyl, thiadiazolyl, thienyl, pyrrolyl, pyridinyl, benzothiazolyl, indolyl, indazolyl, purinyl, quinolyl, isoquinolyl, phthalazinyl, naphthyridinyl, quinoxalinyl, quinazolinyl, quinolizinyl, cinnolinyl, triazolyl, indolizinyl, indolinyl, isoindolinyl, isoindolyl, imidazolidinyl, pteridinyl, thianthrenyl, pyrazolyl, 2H-pyrazolo[3,4-d]pyrimidinyl, 1H-pyrazolo[3,4-d]pyrimidinyl, thieno[2,3-d]pyrimidnyl and the various pyrrolopyridyl radicals.

As used herein, the term 5- to 14-membered heterocyclyl radical embraces typically a non-aromatic, saturated or unsaturated C₅-C₁₄ carbocyclic ring system, preferably C₅-C₁₀ carbocyclic ring system, more preferably C₅-C₆ carbocyclic ring system, in which one or more, for example 1, 2, 3 or 4 of the carbon atoms preferably 1 or 2 of the carbon atoms are replaced by a heteroatom selected from N, O and S. A heterocyclyl radical may be a single ring or two or more fused rings wherein at least one ring contains a heteroatom. When a 5 to 14-membered heterocyclyl radical carries 2 or more substituents, the substituents may be the same or different.

A said optionally substituted 5- to 14-membered heterocyclyl radical is typically unsubstituted or substituted with 1, 2 or 3 substituents which may be the same or different. Typically, the substituents on a 5 to 14-membered heterocyclyl radical are themselves unsubstituted.

Examples of 5- to 14-membered heterocyclyl radicals include piperidyl, pyrrolidyl, pyrrolinyl, piperazinyl, morpholinyl, thiomorpholinyl, pyrrolyl, pyrazolinyl, pirazolidinyl, quinuclidinyl, triazolyl, pyrazolyl, tetrazolyl, imidazolidinyl, imidazolyl, oxiranyl, 4,5-dihydro-oxazolyl, 2-benzofuran-1(3H)-one, 1,3-dioxol-2-one and 3-aza-tetrahydrofuranyl.

Where a 5- to 14-membered heterocyclyl radical carries 2 or more substituents, the substituents may be the same or different.

As used herein, the term 6-membered saturated N-containing heterocyclic group is a C₆ saturated carbocyclic ring system in which one of the carbon atoms is replaced by N and optionally in which 1, 2, or 3, preferably 1 or 2, further carbon atoms are replaced by heteroatoms selected from N and O.

A said 6-membered saturated N-containing heterocyclic group is typically unsubstituted or substituted with 1, 2 or 3 substituents which may be the same or different. Typically, the substituents on a 6-membered saturated N-containing heterocyclic group are themselves unsubstituted, unless otherwise specified.

Examples of 6-membered saturated N-containing heterocyclic group include piperidyl and piperazinyl.

As used herein, the term C₃-C₇ heterocycloalkyl ketone group embraces typically a non-aromatic, saturated or unsaturated C₃-C₇ carbocyclic ring system, in which one of the carbon atoms is replaced by a C═O group and 1, 2 or 3, preferably 1 or 2, more preferably 1, further carbon atoms preferably are replaced by N. Examples include pyridone and pyrrolidone groups.

As used herein, the term aza-bicycloalkyl group having up to 12 carbon atoms denotes a fused ring system consisting of a cycloalkyl group and a N-containing heterocyclyl group, as defined herein

As used herein, the term aza-bicycloalkenyl group having up to 12 carbon atoms embraces an aza-bicycloalkyl group, as defined herein, containing at least one unsaturated carbon-carbon bond.

As used herein, a bicyclyl group containing a monocyclic C₅-C₉ aryl or heteroaryl group bonded directly to a 5- to 9-membered cycloalkyl or heterocyclyl group typically refers to groups where a monocyclic C₅-C₉ aryl or heteroaryl group is bonded to a 5- to 9-membered cycloalkyl or heterocyclyl group by a single bond. Examples include tetrahydroquinolinyl groups, tetrahydroisoquinolinyl groups and chromanyl groups.

As used herein, some of the atoms, radicals, moieties, chains and cycles present in the general structures of the invention are “optionally substituted”. This means that these atoms, radicals, moieties, chains and cycles can be either unsubstituted or substituted in any position by one or more, for example 1, 2, 3 or 4, substituents, whereby the hydrogen atoms bound to the unsubstituted atoms, radicals, moieties, chains and cycles are replaced by chemically acceptable atoms, radicals, moieties, chains and cycles. When two or more substituents are present, each substituent may be the same or different. The substituents are typically themselves unsubstituted.

Typically when a cyclic radical is bridged by an alkylene or alkylenedioxy radical, the bridging alkylene radical is bonded to the ring at non-adjacent atoms.

As used herein, the term halogen atom embraces chlorine, fluorine, bromine and iodine atoms. A halogen atom is typically a fluorine, chlorine or bromine atom, most preferably chlorine or fluorine. The term halo when used as a prefix has the same meaning.

As used herein, the term pharmaceutically acceptable salt embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclyl amines.

Other preferred salts according to the invention are quaternary ammonium compounds wherein an equivalent of an anion (X−) is associated with the positive charge of the N atom. X− may be an anion of various mineral acids such as, for example, chloride, bromide, iodide, sulphate, nitrate, phosphate, or an anion of an organic acid such as, for example, acetate, maleate, fumarate, citrate, oxalate, succinate, tartrate, malate, mandelate, trifluoroacetate, methanesulphonate and p-toluenesulphonate. X− is preferably an anion selected from chloride, bromide, iodide, sulphate, nitrate, acetate, maleate, oxalate, succinate or trifluoroacetate. More preferably X− is chloride, bromide, trifluoroacetate or methanesulphonate.

As used herein, an N-oxide is formed from the tertiary basic amines or imines present in the molecule, using a convenient oxidising agent.

Typically, in the compound of formula (I):
R₁, R₂, R₃, R₄ and R₉ each independently represent a hydrogen atom, a halogen atom, a cyano group, a linear or branched C₁-C₆ alkyl group, a C₂-C₄ alkenyl group, a C₂-C₄ alkynyl group, a C₁-C₄ haloalkyl group, a C₁-C₄ hydroxyalkyl group, a C₃-C₁₀ cycloalkyl group, a C₃-C₁₀ cycloalkenyl group, a monocyclic or polycyclic C₅-C₁₄ aryl group, a 5- to 14-membered heteroaryl group containing at least one heteroatom selected from O, S and N, a 5- to 14-membered heterocyclyl group containing at least one heteroatom selected from O, S and N, a bicyclyl group containing a monocyclic C₅-C₉ aryl or heteroaryl group bonded directly to a 5- to 9-membered cycloalkyl or heterocyclyl group, said heteroaryl or heterocyclyl group containing at least one heteroatom selected from O, S and N, an aza-bicycloalkyl group having up to 12 carbon atoms or an aza-bicycloalkenyl group having up to 12 carbon atoms,
   wherein the alkenyl, alkynyl, haloalkyl, hydroxyalkyl, cycloalkyl, cycloalkenyl, aryl, heteroaryl, heterocyclyl, bicyclyl, aza-bicycloalkyl and aza-bicycloalkenyl groups are unsubstituted or substituted by one or more substituents selected from substituents Ra, and the alkyl groups are unsubstituted or substituted with one or more substituents selected from Rb;
or R₁, R₂, R₃, R₄ and R₉ independently represent a —SR₁₃ group, a —SOR₁₃ group, a —S(O)₂R₁₃ group, a —S(O)₂NR₁₃R₁₄ group, a —NR₁₃S(O)₂R₁₄ group, a —NR₁₃S(O)₂NR₁₄ group, a —OR₁₃ group, a —C(O)OR₁₃ group, a —O—C(O)R₁₃ group, a —C(O)—(CH₂)ₙ—R₁₃ group, a —NR₁₃R₁₄ group, a —C(O)—(CH₂)ₙ—NR₁₃R₁₄ group, a —NR₁₃C(O)—(CH₂)ₙ—R₁₄ group or a —NR₁₃C(O)—(CH₂)ₙ—NR₁₄R₁₅ group, wherein each n is 0, 1 or 2;
or in the case when two adjacent —CR₉ groups are present, two adjacent —CR₉ groups and the carbon atoms to which they are bonded optionally form a C₅-C₁₂ aryl group or a 4- to 12-membered heteroaryl, cycloalkyl or heterocyclyl group, said heteroaryl and heterocyclyl groups containing at least one heteroatom selected from O, S and N, the aryl, heteroaryl, cycloalkyl and heterocyclyl groups being unsubstituted or substituted by one or more substituents selected from a halogen atom, a linear or branched C₁-C₆ alkyl group, a monocyclic or polycyclic C₅-C₁₄ aryl group, a 5- to 14-membered heteroaryl group containing at least one heteroatom selected from O, S and N, or a 5- to 14-membered heterocyclyl group containing at least one heteroatom selected from O, S and N, wherein the alkyl, the aryl, the heteroaryl and the heterocyclyl substituents are unsubstituted or substituted by one or more substituents selected from a halogen atom, a hydroxy group, a cyano group, a linear or branched C₁-C₆ alkyl group or a C₁-C₄ haloalkyl group;
R₅ represents a hydrogen atom, a linear or branched C₁-C₆ alkyl group optionally substituted by one or more substituents selected from a hydroxy group, a cyano group, a C₁-C₄ haloalkyl group, a C₁-C₄ hydroxyalkyl group, a C₃-C₁₀ cycloalkyl group, a phenyl group or a 6 membered, saturated N-containing heterocyclyl ring, or R₅ represents a —S(O)₂R₁₀ group, a —S(O)₂NR₁₀R₁₁ group, a —C(O)OR₁₀ group, a —C(O)—(CH₂)ₙ—R₁₀ group, or a —C(O)—(CH₂)ₙ—NR₁₀R₁₁ group;
R₆ and R₇ each independently represents a hydrogen atom or a linear or branched C₁-C₆ alkyl group optionally substituted by one or more substituents selected from a hydroxy group, a cyano group, a C₁-C₄ haloalkyl group, a C₁-C₄ hydroxyalkyl group, a C₁-C₄ alkoxycarbonyl group, a C₃-C₇ cycloalkyl group, a phenyl group or a 6 membered, saturated N-containing heterocyclyl ring;
R₈ represents a hydrogen atom, a halogen atom, a cyano group, a linear or branched C₁-C₆ alkyl group, a C₂-C₄ alkenyl group, a C₂-C₄ alkynyl group, a C₁-C₄ haloalkyl group, a C₁-C₄ hydroxyalkyl group, a C₃-C₁₀ cycloalkyl group, a C₃-C₁₀ cycloalkenyl group, a monocyclic or polycyclic C₅-C₁₄ aryl group, a 5- to 14-membered heteroaryl group containing at least one heteroatom selected from O, S and N, a 5- to 14-membered heterocyclyl group containing at least one heteroatom selected from O, S and N, a bicyclyl group containing a monocyclic C₅-C₉ aryl or heteroaryl group bonded directly to a 5- to 9-membered cycloalkyl or heterocyclyl group, said heteroaryl or heterocyclyl group containing at least one heteroatom selected from O, S and N, an aza-bicycloalkyl group having up to 12 carbon atoms or a aza-bicycloalkenyl group having up to 12 carbon atoms,
   wherein the alkenyl, alkynyl, haloalkyl, hydroxyalkyl, cycloalkyl, cycloalkenyl, aryl, heteroaryl, heterocyclyl, bicyclyl, aza-bicycloalkyl and aza-bicycloalkenyl groups are unsubstituted or substituted by one or more substituents selected from Ra, —(C₁-C₄ alkyl)-CN groups, or —(C₁-C₄ alkyl)-C(O)NR′R″ groups wherein R′ and R″ are the same or different and are selected from hydrogen atoms and linear or branched C₁-C₄ alkyl groups; and the alkyl groups are unsubstituted or substituted by one or more substituents selected from Rb;
   or R₈ represents a —SR₁₃ group, a —SOR₁₃ group, a —S(O)₂R₁₃ group, a —S(O)₂NR₁₃R₁₄ group, a —NR₁₃S(O)₂R₁₄ group, a —NR₁₃S(O)₂NR₁₄ group, a —OR₁₃ group, a —C(O)OR₁₃ group, a —O—C(O)R₁₃ group, a —C(O)—(CH₂)ₙ—R₁₃ group, a —NR₁₃R₁₄ group, a —C(O)—(CH₂)ₙ—NR₁₃R₁₄ group, a —NR₁₃C(O)—(CH₂)ₙ—R₁₄ group, or a —NR₁₃C(O)—(CH₂)ₙ—NR₁₄R₁₅ group, wherein n is 0, 1 or 2,
or R₈ together with R₅ and the nitrogen atom to which R₅ is bonded form a 4- to 10-membered, saturated heterocyclyl group, which contains, as heteroatoms, one or two nitrogen atoms and which is substituted by a linear or branched C₁-C₆ alkyl group, a monocyclic or polycyclic C₅-C₁₄ aryl group, a 5- to 14-membered heteroaryl group containing at least one heteroatom selected from O, S and N, a 5- to 14-membered heterocyclyl group containing at least one heteroatom selected from O, Sand N, a —SOR₁₀ group, a —C(O)—(CH₂)ₙ—R₁₀ group, or a —C(O)—(CH₂)ₙ—NR₁₀R₁₁ group, wherein each n is 0, 1 or 2,
   wherein the alkyl, aryl, heteroaryl and heterocyclyl groups are unsubstituted or substituted by one or more substituents selected from a halogen atom, a hydroxy group, a cyano group, a linear or branched C₁-C₆ alkyl group or a C₁-C₄ haloalkyl group, and wherein the alkyl groups are unsubstituted or substituted by one or more substituents selected from a halogen atom, a hydroxy group, a cyano group or a C₁-C₄ haloalkyl group;
provided that when m is zero, R₈ is other than a —SR₁₃ group, a —SOR₁₃ group, a —S(O)₂R₁₃ group, a —S(O)₂NR₁₃R₁₄ group, a —NR₁₃S(O)₂R₁₄ group, a —NR₁₃S(O)₂NR₁₄ group, a —OR₁₃ group, a —O—C(O)R₁₃ group, a —NR₁₃R₁₄ group, a —NR₁₃C(O)—(CH₂)ₙ—R₁₄ group, or a —NR₁₃C(O)—(CH₂)ₙ—NR₁₄R₁₅ group,
   wherein
Ra is a halogen atom, a cyano group, a hydroxy group, a linear or branched C₁-C₆ alkyl group, a C₁-C₄ haloalkyl group, a C₁-C₄ hydroxyalkyl group, a C₃-C₇ cycloalkyl or a C₃-C₇ cycloalkenyl group, a monocyclic or polycyclic C₅-C₁₄ aryl group, which aryl group is unsubstituted or substituted by one or more halogen atoms, a 5- to 14-membered heteroaryl group containing at least one heteroatom selected from O, S and N, a 5- to 14-membered heterocyclyl group containing at least one heteroatom selected from O, S and N, a —SR₁₀ group, a —SOR₁₀ group, a —S(O)₂R₁₀ group, a —S(O)₂NR₁₀R₁₁ group, a —NR₁₀S(O)₂R₁₁ group, a —NR₁₀S(O)₂NR₁₁ group, a —OR₁₀ group, a —C(O)OR₁₀ group, a —O—C(O)R₁₀ group, a —C(O)—(CH₂)ₙ—R₁₀ group, a —NR₁₀R₁₁ group,
a —C(O)—(CH₂)ₙ—NR₁₀R₁₁ group, a —NR₁₀C(O)—(CH₂)ₙ—R₁₁ group or a —NR₁₀C(O)—(CH₂)ₙ—NR₁₁R₁₂ group, wherein each n is 0, 1 or 2;
Rb is a cyano group, a C₁-C₄ haloalkyl group, a C₁-C₄ hydroxyalkyl group, a C₃-C₇ cycloalkyl or a C₃-C₇ cycloalkenyl group, a monocyclic or polycyclic C₅-C₁₄ aryl group, which aryl group is unsubstituted or substituted by one or more halogen atoms, a 5- to 14-membered heteroaryl group containing at least one heteroatom selected from O, S and N, a 5- to 14-membered heterocyclyl group containing at least one heteroatom selected from O, S and N, a —SR₁₀ group, a —SOR₁₀ group, a —S(O)₂R₁₀ group, a —S(O)₂NR₁₀R₁₁ group, a —NR₁₀S(O)₂R₁₁ group, a —NR₁₀S(O)₂NR₁₁ group, a —OR₁₀ group, a —C(O)OR₁₀ group, a —O—C(O)R₁₀ group, a —C(O)—(CH₂)ₙ—R₁₀ group, a —NR₁₀R₁₁ group, a —C(O)—(CH₂)ₙ—NR₁₀R₁₁ group, a —NR₁₀C(O)—(CH₂)ₙ—R₁₁ group or a —NR₁₀C(O)—(CH₂)ₙ—NR₁₁R₁₂ group, wherein n is 0, 1 or 2;
R₁₀, R₁₁ and R₁₂ each independently represents a hydrogen atom, a cyano group, a linear or branched C₁-C₆ alkyl group, a C₁-C₄ haloalkyl group, a C₁-C₄ hydroxyalkyl group, a C₁-C₄ alkoxycarbonyl group, a C₃-C₇ cycloalkyl group, a phenyl group, a 5- to 6-membered monocyclic heteroaryl group containing 1, 2 or 3 heteroatoms selected from N, O and S, a 5- to 6-membered, heterocyclyl group containing 1, 2 or 3 nitrogen atoms, a bicyclyl group containing a monocyclic C₅-C₆ aryl or heteroaryl group bonded directly to a 5- to 6-membered cycloalkyl or heterocyclyl group which heteroaryl or heterocyclyl group contains 1, 2 or 3 nitrogen atoms, the haloalkyl, hydroxyalkyl, alkoxycarbonyl, cycloalkyl, phenyl, heteroaryl, heterocyclyl and bicyclyl group being unsubstituted or substituted by one or more substituents selected from substituents Rc, and the alkyl groups being unsubstituted or substituted with one or more substituents selected from substituents Rd;
Rc is a halogen atom, a hydroxy group, a cyano group, a linear or branched C₁-C₆ alkyl group, a C₁-C₄ haloalkyl group, a C₁-C₄ alkoxy group, a C₁-C₄ hydroxyalkyl group, a C₃-C₇ cycloalkyl group, a phenyl group, a 5- to 6-membered monocyclic heteroaryl group containing 1, 2 or 3 nitrogen atoms, a 5- to 6-membered heterocyclyl group containing 1, 2 or 3 nitrogen atoms, or a C₃-C₇ heterocycloalkyl ketone group containing 1, 2 or 3 nitrogen atoms, said phenyl group being unsubstituted or substituted with one or more halogen atoms, and said heteroaryl, heterocyclyl and heterocycloalkyl ketone groups being unsubstituted or substituted by one or more linear or branched C₁-C₃ alkyl groups;
Rd is a cyano group, a C₁-C₄ haloalkyl group, a C₁-C₄ alkoxy group, a C₁-C₄ hydroxyalkyl group, a C₃-C₇ cycloalkyl group, a phenyl group, a 5- to 6-membered monocyclic heteroaryl group containing 1, 2 or 3 nitrogen atoms, a 5- to 6-membered heterocyclyl group containing 1, 2 or 3 nitrogen atoms, or a C₃-C₇ heterocycloalkyl ketone group containing 1, 2 or 3 nitrogen atoms, said phenyl group being unsubstituted or substituted with one or more halogen atoms, and said heteroaryl, heterocyclyl and heterocycloalkyl ketone groups being unsubstituted or substituted by one or more linear or branched C₁-C₃ alkyl groups;
R₁₃, R₁₄, and R₁₅ each independently represents a hydrogen atom, a cyano group, a linear or branched C₁-C₆ alkyl group, a C₁-C₄ haloalkyl group, a C₁-C₄ hydroxyalkyl group, a C₁-C₄ alkoxycarbonyl group, a C₃-C₇ cycloalkyl group, a monocyclic or polycyclic C₅-C₁₄ aryl group, a 5- to 14-membered heteroaryl group containing at least one heteroatom selected from O, S and N, or a 5- to 14-membered heterocyclyl group containing at least one heteroatom selected from O, S and N, wherein the haloalkyl, hydroxyalkyl, alkoxycarbonyl, cycloalkyl, aryl, heteroaryl and heterocyclyl groups are unsubstituted or substituted by one or more substituents selected from the substituents Ra, and the alkyl groups are optionally substituted by one or more substitutents selected from Rb.

In one embodiment, in the compound of formula (I):
R₁, R₂ and R₄ are the same or different and each represent a hydrogen atom, a halogen atom, a hydroxyl group, a cyano group, a linear or branched C₁-C₆ alkyl group, a C₁-C₄ haloalkyl group or C₁-C₄ hydroxyalkyl group;
R₃ represents a hydrogen atom, a halogen atom, a hydroxyl group, a cyano group, a linear or branched C₁-C₆ alkyl group, a C₁-C₄ haloalkyl group, a C₁-C₄ hydroxyalkyl group, a C₃-C₁₀ cycloalkyl group, a 5- to 10-membered heterocyclyl group, a C₆-C₁₀ aryl group, a 5- to 10-membered heteroaryl group, a —C(O)OR′ group or a —C(O)NR″R″′ group, which cycloalkyl, heterocyclyl, aryl and heteroaryl groups are unsubstituted or substituted with one or more halogen atoms or a linear or branched C₁-C₆ alkyl group, a hydroxy group, a cyano group, or a C₁-C₄ alkoxy group; wherein R′, R″ and R″′ are the same or different and each represents a hydrogen atom, a linear or branched C₁-C₆ alkyl group, a C₁-C₄ haloalkyl group or C₁-C₄ hydroxyalkyl group;
R₅ represents a hydrogen atom, a halogen atom, a hydroxyl group, a cyano group, a linear or branched C₁-C₆ alkyl group, a C₁-C₄ haloalkyl group, a C₁-C₄ hydroxyalkyl group or a —(C₁-C₄ alkyl)-(C₃-C₇ cycloalkyl) group, or R₅ together with R₈ and the nitrogen atom to which R₅ is bonded form a 5- to 9-membered, saturated heterocyclyl group, which contains, as heteroatoms, one or two nitrogen atoms and which heterocyclyl ring is unsubstituted or substituted with a 5- or 6-membered heteroaryl group containing one or two nitrogen atoms, a —C(O)—(CH₂)ₙ—R′ group or a —C(O)—(CH₂), —NR″R″′ group, wherein n is 0, 1 or 2, R′ represents a hydrogen atom, or a linear or branched C₁-C₆ alkyl group, a C₁-C₄ haloalkyl group, a C₁-C₄ hydroxyalkyl group, or a cyano group and R″ and R″′ are the same or different and each represents a hydrogen atom, a linear or branched C₁-C₆ alkyl group, a C₁-C₄ haloalkyl group, or C₁-C₄ hydroxyalkyl group;
R₆ and R₇ are the same or different and each represent a hydrogen atom, a linear or branched C₁-C₆ alkyl group, a C₁-C₄ haloalkyl group, a C₁-C₄ hydroxyalkyl group or a —(C₁-C₄ alkyl)-Het-(C₁-C₄ alkyl) group;
R₉ is a hydrogen atom, a halogen atom, a hydroxyl group, a cyano group, a linear or branched C₁-C₆ alkyl group, a C₁-C₄ alkoxy group, a C₁-C₄ haloalkyl group, a C₁-C₄ hydroxyalkyl group, a C₃-C₇ cycloalkyl group, a 5- to 10-membered heterocyclyl group, or a 5- to 10-membered heteroaryl group, which heterocyclyl and heteroaryl groups are unsubstituted or substituted with one or more halogen atoms or a linear or branched C₁-C₉ alkyl group, a cyano group, a hydroxy group, a carboxyl group or a C₁-C₄ alkoxy substituents, or R₉ is a -Het-R′, Y′—R″′ or —C(O)—Het-R′ group, or in the case when two adjacent —CR₉ groups are present, the two adjacent —CR₉ groups and the carbon atoms to which they are bonded optionally form a C₆-C₁₀ aryl group which is unsubstituted or substituted by one or more substituents selected from a halogen atom, a linear or branched C₁-C₆ alkyl group, a hydroxy group or a C₁-C₄ alkoxy group;
R₈ together with R₅ and the nitrogen atom to which R₅ is bonded form a said 5- to 9-membered heterocyclyl ring, or R₈ is a hydrogen atom, a linear or branched C₁-C₆ alkyl group, a C₁-C₄ haloalkyl group, a C₁-C₄ hydroxyalkyl group, a C₃-C₁₀ cycloalkyl group, a 5- to 10-membered heterocyclyl group, a 4- to 10-membered heterocycyl ring containing 1, 2 or 3 nitrogen atoms, which ring is substituted one, two or three oxo groups, a C₆-C₁₀ aryl group, a 5- to 10-membered heteroaryl group, -L-Het-R″′, -L-A, -A-SO₂—R′, -A-SO—R″′, -A-A′, -A-L-C(O)NR′R″,
-A-L-CN, -A-C(O)—Het′-L-CN, -A-C(O)—NR′R″, -A-C(O)_{z}-A″, -A-C(O)—R″′, -A-CO₂—R′, -A-C(O)_{z}-L-A″′, -A-C(O)_{z}-L-CN, -A-C(O)-A′-A″, -A-C(O)-L-R′, -A-C(O)-L-CN, -A-Het-L-CN, -A-C(O)-L-Het-A′, -A-C(O)-L-Het-L-A′, A-C(O)-L-Het-L-R″′, A-C(O)-L-Het-C(O)-A′, or -A-C(O)_{z}-L-Het-R′ group, wherein z is 1 or 2, R′ and R″ are the same or different and each represents a hydrogen atom, a C₁-C₄ haloalkyl group, a C₁-C₄ hydroxyalkyl group group, or a linear or branched C₁-C₆ alkyl group, which alkyl group is unsubstituted or substituted by a C₁-C₂ alkoxy group or a 5- or 6-membered heterocyclyl group, and R″′ represents a linear or branched C₁-C₆ alkyl group, a C₁-C₄ haloalkyl group or a C₁-C₄ hydroxyalkyl group, the heterocyclyl and heteroaryl groups being optionally fused to a phenyl group, and wherein the cycloalkyl, heterocyclyl, aryl and heteroaryl groups being unsubstituted or substituted with one or more halogen atoms, a hydroxyl group, a cyano, C₁-C₄ alkoxy group, or a linear or branched C₁-C₄ alkyl group, which alkyl group is unsubstituted or substituted by a cyano group, and wherein
L is a linear or branched C₁-C₆ alkylene group, which is unsubstituted or substituted by one or two hydroxyl groups,
Het represents O or NR, and Het′ represents NR, wherein R is a hydrogen atom, a straight or branched C₁-C₄ alkyl group, a C₁-C₄ haloalkyl group, or C₁-C₄ hydroxyalkyl group,
Y′ represents —C(O)—, SO or SO₂,
A, A′, A″ and A″′ are the same or different and each represent a C₃-C₁₀ cycloalkyl group, a 5- to 10-membered heterocyclyl group, a 4- to 10-membered heterocycyl ring containing 1, 2 or 3 nitrogen atoms, which ring is substituted one, two or three oxo groups, a C₆-C₁₀ aryl group, a 5- to 10-membered heteroaryl group or a bicyclyl group containing a monocyclic C₅-C₆ aryl or heteroaryl group bonded directly to a 5- to 6-membered cycloalkyl or heterocyclyl group, the cycloalkyl, heterocyclyl, aryl heteroaryl and bycyclyl groups being unsubstituted or substituted with one or more halogen atoms, hydroxyl groups, cyano groups, linear or branched C₁-C₄ alkyl groups, C₁-C₄ haloalkyl groups, C₁-C₄ hydroxyalkyl groups, or C₁-C₄ alkoxy groups.

Typically, in this embodiment, in the compound of formula (I):
R₁, R₂ and R₄ are the same or different and each represent a hydrogen atom, a halogen atom, a hydroxyl group, a cyano group, a linear or branched C₁-C₆ alkyl group, a C₁-C₄ haloalkyl group or C₁-C₄ hydroxyalkyl group;
R₃ represents a hydrogen atom, a halogen atom, a hydroxyl group, a cyano group, a linear or branched C₁-C₆ alkyl group, a C₁-C₄ haloalkyl group, a C₁-C₄ hydroxyalkyl group, a C₃-C₁₀ cycloalkyl group, a 5- to 10-membered heterocyclyl group, a C₆-C₁₀ aryl group, a 5- to 10-membered heteroaryl group, a —C(O)OR′ group or a —C(O)NR″R″′ group, which cycloalkyl, heterocyclyl, aryl and heteroaryl groups are unsubstituted or substituted with one or more halogen atoms or a linear or branched C₁-C₆ alkyl group, a hydroxy group, a cyano group, or a C₁-C₄ alkoxy group; wherein R′, R″ and R″′ are the same or different and each represents a hydrogen atom, a linear or branched C₁-C₆ alkyl group, a C₁-C₄ haloalkyl group or C₁-C₄ hydroxyalkyl group;
R₅ represents a hydrogen atom, a halogen atom, a hydroxyl group, a cyano group, a linear or branched C₁-C₆ alkyl group, a C₁-C₄ haloalkyl group, a C₁-C₄ hydroxyalkyl group or a —(C₁-C₄ alkyl)-(C₃-C₇ cycloalkyl) group, or R₅ together with R₈ and the nitrogen atom to which R₅ is bonded form a 5- to 9-membered, saturated heterocyclyl group, which contains, as heteroatoms, one or two nitrogen atoms and which heterocyclyl ring is unsubstituted or substituted with a —C(O)—(CH₂)ₙ—R′ group or a —C(O)—(CH₂)ₙ—NR″R″′ group, wherein n is 0, 1 or 2, R′ represents a hydrogen atom, or a linear or branched C₁-C₆ alkyl group, a C₁-C₄ haloalkyl group, a C₁-C₄ hydroxyalkyl group, or a cyano group and R″ and R″′ are the same or different and each represents a hydrogen atom, a linear or branched C₁-C₆ alkyl group, a C₁-C₄ haloalkyl group, or C₁-C₄ hydroxyalkyl group;
R₆ and R₇ are the same or different and each represent a hydrogen atom, a linear or branched C₁-C₆ alkyl group, or a C₁-C₄ haloalkyl group or a C₁-C₄ hydroxyalkyl group;
R₉ is a hydrogen atom, a halogen atom, a hydroxyl group, a cyano group, a linear or branched C₁-C₆ alkyl group, a C₁-C₄ haloalkyl group, a C₁-C₄ hydroxyalkyl group, a 5- to 10-membered heterocyclyl group, or a 5- to 10-membered heteroaryl group, which heterocyclyl and heteroaryl groups are unsubstituted or substituted with one or more halogen atoms or a linear or branched C₁-C₆ alkyl group, a cyano group, a hydroxy group or a C₁-C₄ alkoxy substituents, or in the case when two adjacent —CR₉ groups are present, the two adjacent —CR₉ groups and the carbon atoms to which they are bonded optionally form a C₆-C₁₀ aryl group which is unsubstituted or substituted by one or more substituents selected from a halogen atom, a linear or branched C₁-C₆ alkyl group, a hydroxy group or a C₁-C₄ alkoxy group;
R₈ together with R₅ and the nitrogen atom to which R₅ is bonded form a said 5- to 9-membered heterocyclyl ring, or R₈ is a hydrogen atom, a linear or branched C₁-C₆ alkyl group, a C₁-C₄ haloalkyl group, a C₁-C₄ hydroxyalkyl group, a C₃-C₁₀ cycloalkyl group, a 5- to 10-membered heterocyclyl group, a C₆-C₁₀ aryl group, a 5- to 10-membered heteroaryl group, -L-Het-R″′, -L-A, -A-SO₂—R′, -A-SO—R″′, -A-A′, -A-L-C(O)NR′R″, -A-L-CN, -A-C(O)—Het′-L-CN, -A-C(O)—NR′R″, -A-C(O)_{z}-A″, -A-C(O)—R″′, -A-CO₂—R′, -A-C(O)_{z}-L-A″′, -A-C(O)_{z}-L-CN, or -A-C(O)_{z}-L-Het-R′ group, wherein z is 1 or 2, R′ and R″ are the same or different and each represents a hydrogen atom or linear or a branched C₁-C₆ alkyl group, a C₁-C₄ haloalkyl group or a C₁-C₄ hydroxyalkyl group group, and R″′ represents a linear or branched C₁-C₆ alkyl group, a C₁-C₄ haloalkyl group or a C₁-C₄ hydroxyalkyl group, the heterocyclyl and heteroaryl groups being optionally fused to a phenyl group, and wherein the cycloalkyl, heterocyclyl, aryl and heteroaryl groups being unsubstituted or substituted with one or more halogen atoms, a hydroxyl group, a cyano, a linear or branched C₁-C₄ alkyl group, or C₁-C₄ alkoxy group, and wherein
L is a linear or branched C₁-C₆ alkylene group;
Het represents O or NR, and Het′ represents NR, wherein R is a hydrogen atom, a straight or branched C₁-C₄ alkyl group, a C₁-C₄ haloalkyl group, or C₁-C₄ hydroxyalkyl group;
A, A′, A″ and A″′ are the same or different and each represent a C₃-C₁₀ cycloalkyl group, a 5- to 10-membered heterocyclyl group, a C₆-C₁₀ aryl group, or a 5- to 10-membered heteroaryl group, the cycloalkyl, heterocyclyl, aryl and heteroaryl groups being unsubstituted or substituted with one or more halogen atoms, a hydroxyl group, a cyano group, a linear or branched C₁-C₄ alkyl group, or a C₁-C₄ alkoxy group.

Typically, R₁, R₂ and R₄ are the same or different and each represent a hydrogen atom, a halogen atom, or a hydroxy or linear or branched C₁-C₄ alkyl group. Preferably, R₁, R₂ and R₄ are the same and each represents a hydrogen atom.

Typically, R₃ represents a hydrogen atom, a halogen atom, or a hydroxy, cyano, linear or branched C₁-C₄ alkyl, C₃-C₆ cycloalkyl, phenyl, 5- to 6-membered heteroaryl, C(O)OR′, or —C(O)NR″R″′ group, which cycloalkyl, phenyl and heteroaryl groups are unsubstituted or substituted with 1, 2 or 3 halogen atoms or linear or branched C₁-C₂ alkyl, hydroxy, cyano or C₁-C₂ alkoxy substituents, wherein R′, R″ and R″′ are the same or different and each represents a hydrogen atom, or a linear or branched C₁-C₂ alkyl group. Preferably, R₃ represents a hydrogen atom, a halogen atom, or a cyano, C₃-C₄ cycloalkyl, phenyl, pyridyl, pyrazolyl, or C(O)OR′ group, which cycloalkyl, phenyl, pyridyl and pyrazolyl groups are unsubstituted or substituted with 1 or 2 halogen atoms, wherein R′ represents a hydrogen atom, or a linear or branched C₁-C₂ alkyl group.

Typically, R₅ represents a hydrogen atom, a halogen atom, or a hydroxy, linear or branched C₁-C₄ alkyl, or —(C₁-C₄ alkyl)-(C₃-C₆ cycloalkyl) group, or R₅ together with R₈ and the nitrogen atom to which R₅ is bonded form a 5- to 7-membered, saturated heterocyclyl group, which contains, as heteroatoms, one or two nitrogen atoms and which heterocyclyl ring is unsubstituted or substituted with —C(O)—(CH₂)ₙ—R′, —C(O)—(CH₂)ₙ—NR″R″′ or 5- or 6-membered heteroaryl group, wherein n is 0 or 1, R′ represents a hydrogen atom, or a linear or branched C₁-C₄ alkyl, or a cyano group and R″ and R″′ are the same or different and each represents a hydrogen atom, or a linear or branched C₁-C₄ alkyl group. More typically, R₅ represents a hydrogen atom, a halogen atom, or a hydroxy, linear or branched C₁-C₄ alkyl, or —(C₁-C₄ alkyl)-(C₃-C₆ cycloalkyl) group, or R₅ together with R₈ and the nitrogen atom to which R₅ is bonded form a 5- to 7-membered, saturated heterocyclyl group, which contains, as heteroatoms, one or two nitrogen atoms and which heterocyclyl ring is unsubstituted or substituted with —C(O)—(CH₂), —R′ or —C(O)—(CH₂)ₙ—NR″R″′, wherein n is 0 or 1, R′ represents a hydrogen atom, or a linear or branched C₁-C₄ alkyl, or a cyano group and R″ and R″′ are the same or different and each represents a hydrogen atom, or a linear or branched C₁-C₄ alkyl group.

Preferably, R₅ represents a hydrogen atom, or a hydroxy, linear or branched C₁-C₂ alkyl, or —(C₁-C₂ alkyl)-(C₃-C₄ cycloalkyl) group, or R₅ together with R₈ and the nitrogen atom to which R₅ is bonded form a 7-membered heterocyclyl ring, which contains as heteroatoms one or two nitrogen atoms, which heterocyclyl ring is unsubstituted or substituted with a —C(O)—CH₂—R′ group wherein R′ represents a linear or branched C₁-C₂ alkyl or cyano group, a —C(O)—CH₂—NEt₂ group or a pyridyl group. More preferably, R₅ represents a hydrogen atom, or a hydroxy, linear or branched C₁-C₂ alkyl, or —(C₁-C₂ alkyl)-(C₃-C₄ cycloalkyl) group, or R₅ together with R₈ and the nitrogen atom to which R₅ is bonded form a 7-membered heterocyclyl ring, which contains as heteroatoms one or two nitrogen atoms, which heterocyclyl ring is unsubstituted or substituted with a —C(O)—CH₂—R′ group wherein R′ represents a linear or branched C₁-C₂ alkyl or cyano group.

Typically, R₆ and R₇ are the same or different and each represent a hydrogen atom, a linear or branched C₁-C₄ alkyl group or a (C₁-C₂ alkyl)-O—(C₁-C₂ alkyl) group. More typically, R₆ and R₇ are the same or different and each represent a hydrogen atom, or a linear or branched C₁-C₄ alkyl group.

Preferably, R₆ and R₇ are the same or different and each represent a hydrogen atom, or a C₁-C₃ alkyl group or a methoxymethyl group. More preferably, R₆ and R₇ are the same or different and each represent a hydrogen atom, or a C₁-C₂ alkyl group.

Typically, R₉ is a hydrogen atom, a halogen atom, or a hydroxy, linear or branched C₁-C₄ alkyl, C₁-C₄ alkoxy, C₃-C₇ cycloalkyl, 5- to 6-membered heterocyclyl, or 5- to 6-membered heteroaryl group, which heterocyclyl and heteroaryl groups are unsubstituted or substituted with 1, 2 or 3 halogen atoms or linear or branched C₁-C₄ alkyl, carboxyl, or C₁-C₄ alkoxy substituents, or in the case when two adjacent —CR₉ groups are present, the two adjacent —CR₉ groups and the carbon atoms to which they are bonded optionally form a benzene ring which is unsubstituted or substituted by 1, 2 or 3 substituents selected from a halogen atom, or a linear or branched C₁-C₂ alkyl, or C₁-C₂ alkoxy substituents, or R₉ is a -Het-R′, Y′—R″′ or —C(O)—Het-R′ group. More typically, R₉ is a hydrogen atom, a halogen atom, or a hydroxy, linear or branched C₁-C₄ alkyl, 5- to 6-membered heterocyclyl, or 5- to 6-membered heteroaryl group, which heterocyclyl and heteroaryl groups are unsubstituted or substituted with 1, 2 or 3 halogen atoms or linear or branched C₁-C₄ alkyl, or C₁-C₄ alkoxy substituents, or in the case when two adjacent —CR₉ groups are present, the two adjacent —CR₉ groups and the carbon atoms to which they are bonded optionally form a benzene ring which is unsubstituted or substituted by 1, 2 or 3 substituents selected from a halogen atom, or a linear or branched C₁-C₂ alkyl, or C₁-C₂ alkoxy substituents.

Preferably, R₉ is a hydrogen atom, a halogen atom, or a C₁-C₂ alkyl, piperazine, pyridone or pyridine group, which piperazine, pyridone and pyridine groups are unsubstituted or substituted with 1 or 2 halogen atoms or C₁-C₂ alkoxy substituents, or R₉ is a -Het-R′, Y′—R″′ or —C(O)—Het-R′ group, or in the case when two adjacent —CR₉ groups are present, the two adjacent —CR₉ groups and the carbon atoms to which they are bonded optionally form a benzene ring which is unsubstituted. More preferably, R₉ is a hydrogen atom, a halogen atom, or a C₁-C₂ alkyl, piperazine, pyridone or pyridine group, which piperazine, pyridone and pyridine groups are unsubstituted or substituted with 1 or 2 halogen atoms or C₁-C₂ alkoxy substituents, or in the case when two adjacent —CR₉ groups are present, the two adjacent —CR₉ groups and the carbon atoms to which they are bonded optionally form a benzene ring which is unsubstituted.

Typically, in the compound of formula (I) R₈ represents a linear or branched C₁-C₆ alkyl group, a C₁-C₄ haloalkyl group, a C₁-C₄ hydroxyalkyl group, a C₃-C₁₀ cycloalkyl group, a C₆-C₁₀ aryl group, a 5- to 10-membered heteroaryl group containing 1, 2 or 3 heteroatoms selected from N, O and S, a 5- to 10-membered heterocyclyl group containing 1, 2 or 3 heteroatoms selected from N, O and S, a 5- to 7-membered heterocyclyl ring containing 1, 2 or 3 nitrogen atoms which ring is substituted by one or two oxo groups, or R₈ is -L-Het-R″′, -L-A, -A-A′, -A-L-C(O)NR′R″, -A-L-CN, -A-C(O)-Het′-L-CN, -A-C(O)—NR′R″, -A-C(O)_{z}-A″,
-A-C(O)—R″′, -A-CO₂—R′, -A-C(O)_{z}-L-A″′, -A-C(O)_{z}-L-R″, -A-C(O)_{z}-L-CN, -A-C(O)-A′-A″, -A-C(O)-L-R′, -A-C(O)-L-CN, -A-Het-L-CN, -A-C(O)-L-Het-A′, -A-C(O)-L-Het-L-A′, -A-C(O)-L-Het-L-R″′, A-C(O)-L-Het-C(O)-A′, or -A-C(O)_{z}L-Het-R′ group, wherein z is 1 or 2, R′ and R″ are the same or different and each represents a hydrogen atom or linear or a branched C₁-C₆ alkyl group which alkyl group is unsubstituted or substituted by a C₁-C₆ alkoxy group or a 5 or 6 membered heterocyclyl group, a C₁-C₄ haloalkyl group or a C₁-C₄ hydroxyalkyl group, and R″′ represents a linear or branched C₁-C₆ alkyl group, a C₁-C₄ haloalkyl group or a C₁-C₄ hydroxyalkyl group, the heterocyclyl and heteroaryl groups being optionally fused to a phenyl group, and wherein the cycloalkyl, heterocyclyl, aryl and heteroaryl groups being unsubstituted or substituted by one or more substituents selected from a halogen atom, a hydroxyl group, a cyano group, a linear or branched C₁-C₄ alkyl group which alkyl group is unsubstituted or substituted by a cyano group, or a C₁-C₄ alkoxy group.

More typically, in the compound of formula (I) R₈ represents a linear or branched C₁-C₆ alkyl group, a C₁-C₄ haloalkyl group, a C₁-C₄ hydroxyalkyl group, a C₃-C₁₀ cycloalkyl group, a C₆-C₁₀ aryl group, a 5- to 10-membered heteroaryl group containing 1, 2 or 3 heteroatoms selected from N, O and S, a 5- to 10-membered heterocyclyl group containing 1, 2 or 3 heteroatoms selected from N, O and S, -L-Het-R″′, -L-A, -A-A′, -A-L-C(O)NR′R″, -A-L-CN, -A-C(O)—Het′-L-CN, -A-C(O)—NR′R″, -A-C(O)_{z}A″, -A-C(O)—R″′, -A-CO₂—R′, -A-C(O)_{z}-L-A″′, -A-C(O)_{z}L-R″′, -A-C(O)_{z}L-CN, or -A-C(O)_{z}-L-Het-R′ group, wherein z is 1 or 2, R′ and R″ are the same or different and each represents a hydrogen atom or linear or a branched C₁-C₆ alkyl group, a C₁-C₄ haloalkyl group or a C₁-C₄ hydroxyalkyl group, and R″′ represents a linear or branched C₁-C₆ alkyl group, a C₁-C₄ haloalkyl group or a C₁-C₄ hydroxyalkyl group, the heterocyclyl and heteroaryl groups being optionally fused to a phenyl group, and wherein the cycloalkyl, heterocyclyl, aryl and heteroaryl groups being unsubstituted or substituted by one or more substituents selected from a halogen atom, a hydroxyl group, a cyano group, a linear or branched C₁-C₄ alkyl group, or a C₁-C₄ alkoxy group.

Typically, L is a linear or branched C₁-C₆ alkylene group which is unsubstituted or substituted by one or two hydroxyl groups. More typically, L is a linear or branched C₁-C₆ alkylene group. Preferably, L is a linear or branched C₁-C₆ alkylene group which is unsubstituted or substituted by one or two hydroxyl groups. More preferably, L is a linear or branched C₁-C₆ alkylene group.

Typically, Het represents O or NR and Het′ represents NR, wherein R is a hydrogen atom or a straight or branched C₁-C₄ alkyl group, preferably a hydrogen atom or a straight or branched C₁-C₂ alkyl group. More Typically, Het represents O or NR and Het′ represents NR, wherein R is a hydrogen atom or a straight or branched C₁-C₄ alkyl group, preferably a hydrogen atom or a straight or branched C₁-C₂ alkyl group. Preferably, Het represents O, NH or N(CH₃). More preferably, Het represents O.

Typically, Y is a SO₂ group.

Typically, A, A′, A″ and A″′ are the same or different and each represent a C₃-C₆ cycloalkyl, 5- to 6-membered heterocyclyl, chromanyl, phenyl, 5- to 9-membered heteroaryl group, the cycloalkyl, heterocyclyl, phenyl and heteroaryl groups being unsubstituted or substituted with 1, 2 or 3 halogen atoms, or hydroxy, cyano, linear or branched C₁-C₂ alkyl, C₁-C₂ haloalkyl, C₁-C₂ hydroxyalkyl, or C₁-C₂ alkoxy groups. More typically, A, A′, A″ and A′″ are the same or different and each represent a C₃-C₆ cycloalkyl, 5- to 6-membered heterocyclyl, phenyl, 5- to 6-membered heteroaryl group, the cycloalkyl, heterocyclyl, phenyl and heteroaryl groups being unsubstituted or substituted with 1, 2 or 3 halogen atoms, or hydroxy, cyano, linear or branched C₁-C₂ alkyl, or C₁-C₂ alkoxy groups.

Typically, A is a 5- to 6-membered heterocyclyl group, 5- to 6-membered heteroaryl group, phenyl or C₃-C₆ cycloalkyl group, said heterocyclyl, heteroaryl, phenyl and cycloalkyl groups being unsubstituted or substituted with 1, 2 or 3, preferably 1 or 2, halogen atoms or hydroxy or C₁-C₂ alkyl groups. More typically, A is a 5- to 6-membered heterocyclyl group, phenyl or C₃-C₆ cycloalkyl group, said heterocyclyl, phenyl and cycloalkyl groups being unsubstituted or substituted with 1, 2 or 3, preferably 1 or 2, halogen atoms or hydroxy or C₁-C₂ alkyl groups.

Preferably, A is a piperidinyl, pyridyl, phenyl or cyclohexyl group, which piperidinyl, pyridyl, phenyl and cyclohexyl groups are unsubstituted or substituted with one C₁-C₂ alkyl group. More preferably, A is a piperidinyl, phenyl or cyclohexyl group, which piperidinyl, phenyl and cyclohexyl groups are unsubstituted or substituted with one C₁-C₂ alkyl group.

Typically, A′ is a C₃-C₆ cycloalkyl group, a 5-, 6- or 9-membered heteroaryl group, a 5- to 6-membered heterocyclyl group, a chromanyl or phenyl group, which cycloalkyl, heteroaryl, heterocyclyl, chromanyl or phenyl group is unsubstituted or substituted with 1, 2 or 3, halogen atoms, or cyano, hydroxy, C₁-C₂ alkyl, C₁-C₂ hydroxyalkyl, C₁-C₂ haloalkyl C₁-C₂ alkoxy or phenyl groups. More typically, A′ is phenyl group, which is unsubstituted or substituted with 1, 2 or 3, halogen atoms, or cyano, hydroxy or C₁-C₂ alkyl groups.

Preferably, A′ is a phenyl group which is unsubstituted or substituted with 1 or 2 halogen atoms, hydroxymethyl groups, methoxy groups or cyano groups; a C₃-C₆ cycloalkyl group which is unsubstituted or substituted by one or two halogen atoms, hydroxyl groups, methyl groups or trifluoromethyl groups; a pyrimidinedione group; a triazole group; a pyrazole group; a thiadiazole group which is unsubstituted or substituted by a methyl group; a pyrimidine group which is unsubstituted or substituted by one or two hydroxyl groups or halogen atoms; an indole group; a tetrahydropyran group; a benzimidazole group; a chromanyl group; a tetrazole group; a thiazole group which is unsubstituted or substituted by a cyano group; or a doixolyl group which is unsubstituted or substituted by one or two methyl groups. More preferably, A′ is a phenyl group, which is unsubstituted or substituted with 1 or 2 halogen atoms or cyano groups.

Typically, A″ is a 5- to 6-membered heterocyclyl, C₃-C₆ cycloalkyl, phenyl or 5- or 6-membered heteroaryl group, which heterocyclyl, cycloalkyl and heteroaryl groups are unsubstituted or substituted with 1, 2 or 3, halogen atoms, or cyano, hydroxy or C₁-C₂ alkyl groups. More typically, A″ is a 5- to 6-membered heterocyclyl, C₃-C₆ cycloalkyl or 5- or 6-membered heteroaryl group, which heterocyclyl, cycloalkyl and heteroaryl groups are unsubstituted or substituted with 1, 2 or 3, halogen atoms, or cyano, hydroxy or C₁-C₂ alkyl groups.

Preferably, A″ is a pyrrolidinyl, cyclopropyl phenyl or pyridinyl group, which pyrrolidinyl, cyclopropyl and pyridinyl groups are unsubstituted or substituted with 1 or 2 halogen atoms or cyano groups. More preferably, A″ is a pyrrolidinyl, cyclopropyl or pyridinyl group, which pyrrolidinyl, cyclopropyl and pyridinyl groups are unsubstituted or substituted with 1 or 2 halogen atoms or cyano groups.

Typically, A′″ is a 5- to 6-membered heteroaryl group, which heteroaryl group is unsubstituted or substituted with 1, 2 or 3, preferably 1 or 2 halogen atoms or hydroxy or C₁-C₂ alkyl groups. More typically, A″′ is a 5- to 6-membered heteroaryl group, which heteroaryl group is unsubstituted or substituted with 1, 2 or 3, preferably 1 or 2 halogen atoms or hydroxy or C₁-C₂ alkyl groups.

Preferably, A″′ is an imidazolyl group. More preferably, A′″ is an imidazolyl group.

Typically, R₈ together with R₅ and the nitrogen atom to which R₅ is bonded form a said 5- to 7-membered heterocyclyl ring, or R₈ is a hydrogen atom, or a linear or branched C₁-C₆ alkyl, C₆-C₁₀ cycloalkyl, 5- to 6-membered heterocyclyl, phenyl, 5- to 6-membered heteroaryl group, -L-Het-R″′, -L-A, -A-SO₂—R′, -A-A′, -A-L-C(O)NR′R″, -A-L-CN, -A-C(O)—Het′-L-CN, -A-C(O)—NR′R″^{I}, -A-C(O)-A″, -A-C(O)—R″′, -A-C(O)-L-A″′, -A-C(O)-L-CN, -A-C(O)-A′-A″, -A-C(O)-L-R′, -A-C(O)-L-CN, -A-Het-L-CN, -A-C(O)-L-Het-A′, -A-C(O)-L-Het-L-A′, -A-C(O)-L-Het-L-R″′, A-C(O)-L-Het-C(O)-A′, or -A-C(O)-L-Het-R′ group, wherein R′ and R″ are the same or different and each represents a hydrogen atom, a C₁-C₂ haloalkyl group, a C₁-C₄ hydroxyalkyl group, or a linear or branched C₁-C₆ alkyl group which alkyl group is unsubstituted by a C₁-C₂ alkoxy group or a 5 or 6 membered heterocycl group, R″′ represents a linear or branched C₁-C₆ alkyl, C₁-C₂ haloalkyl or C₁-C₄ hydroxyalkyl group, the 5- to 6-membered heterocyclyl group being optionally fused to a phenyl group, and wherein the cycloalkyl, heterocyclyl, phenyl and heteroaryl groups are unsubstituted or substituted with 1, 2 or 3 halogen atoms, or hydroxy, cyano, C₁-C₂ alkyl, or C₁-C₂ alkoxy groups, and L, Het, Het′, A, A′, A″ and A″′ are as defined above.

More typically, R₈ together with R₅ and the nitrogen atom to which R₅ is bonded form a said 5- to 7-membered heterocyclyl ring, or R₈ is a hydrogen atom, or a linear or branched C₁-C₆ alkyl, C₆-C₁₀ cycloalkyl, 5- to 6-membered heterocyclyl, phenyl, 5- to 6-membered heteroaryl group, -L-Het-R″′, -L-A, -A-SO₂—R′, -A-A′, -A-L-C(O)NR′R″, -A-L-CN, -A-C(O)—Het′-L-CN, -A-C(O)—NR′R″^{I}, -A-C(O)-A″, -A-C(O)—R′″, -A-C(O)-L-A″′, -A-C(O)-L-CN, or -A-C(O)-L-Het-R′ group, wherein R′ and R″ are the same or different and each represents a hydrogen atom or linear or branched C₁-C₆ alkyl, C₁-C₂ haloalkyl or C₁-C₄ hydroxyalkyl group, R″′ represents a linear or branched C₁-C₆ alkyl, C₁-C₂ haloalkyl or C₁-C₄ hydroxyalkyl group, the 5- to 6-membered heterocyclyl group being optionally fused to a phenyl group, and wherein the cycloalkyl, heterocyclyl, phenyl and heteroaryl groups are unsubstituted or substituted with 1, 2 or 3 halogen atoms, or hydroxy, cyano, C₁-C₂ alkyl, or C₁-C₂ alkoxy groups, and L, Het, Het′, A, A′, A″ and A″′ are as defined above.

Preferably, R₈ together with R₅ and the nitrogen atom to which R₅ is bonded form a said 7-membered heterocyclyl ring, or R₈ is a linear or branched C₁-C₃ alkyl, C₃-C₆ cycloalkyl, adamantyl, piperidinyl, phenyl, pyrrolidine, pyrrolidinone, pyridine, tetrahydroquinoline, pyranyl, -L-Het-R″′, -L-A, -A-SO₂—R′, -A-A′, -A-L-C(O)NR′R″, -A-L-CN, -A-C(O)—Het′-L-CN, -A-C(O)—NR′R″^{I}, -A-C(O)-A″, -A-C(O)—R″′, -A-C(O)-L-A′″, -A-C(O)-L-CN, -A-C(O)-A′-A″, -A-C(O)-L-R′, -A-C(O)-L-CN, -A-Het-L-CN, -A-C(O)-L-Het-A′, -A-C(O)-L-Het-L-A′, A-C(O)-L-Het-L-R″′, A-C(O)-L-Het-C(O)-A′, or -A-C(O)-L-Het-R′ group, wherein R′ and R″ are the same or different and each represents a hydrogen atom or linear or branched C₁-C₃ alkyl group which alkyl group is unsubstituted or substituted by a C₁-C₂ alkoxy group or a 5 or 6 membered heterocycl group, and R′″ represents a linear or branched C₁-C₆ alkyl, C₁-C₂ haloalkyl or C₁-C₄ hydroxyalkyl group, the pyranyl group being optionally fused to a phenyl group, and wherein the C₃-C₆ cycloalkyl, adamantyl, piperidinyl, phenyl pyrrolidine, pyrrolidinone, pyridine, tetrahydroquinoline, and pyranyl groups are unsubstituted or substituted with 1 or 2 halogen atoms, or hydroxy, C₁-C₂ alkyl, or C₁-C₂ alkoxy groups, and L, Het, Het′, A, A′, A″ and A″′ are as defined above.

More preferably, R₈ together with R₅ and the nitrogen atom to which R₅ is bonded form a said 7-membered heterocyclyl ring, or R₈ is a linear or branched C₁-C₃ alkyl, cyclohexyl, adamantyl, piperidinyl, phenyl, pyranyl, -L-Het-R″′, -L-A, -A-SO₂—R′, -A-A′, -A-L-C(O)NR′R″, -A-L-CN, -A-C(O)—Het′-L-CN, -A-C(O)—NR′R″^{I}, -A-C(O)-A″, -A-C(O)—R″′, -A-C(O)-L-A″′, -A-C(O)-L-CN, or -A-C(O)-L-Het-R′ group, wherein R′ and R″ are the same or different and each represents a hydrogen atom or linear or branched C₁-C₃ alkyl group, and R″′ represents a linear or branched C₁-C₆ alkyl, C₁-C₂ haloalkyl or C₁-C₄ hydroxyalkyl group, the pyranyl group being optionally fused to a phenyl group, and wherein the cyclohexyl, adamantyl, piperidinyl, phenyl and pyranyl groups are unsubstituted or substituted with 1 or 2 halogen atoms, or hydroxy, C₁-C₂ alkyl, or C₁-C₂ alkoxy groups, and L, Het, Het′, A, A′, A″ and A″′ are as defined above.

Preferably, in the compound of formula (I) Z is a group NR₅ and R₅ is as defined above.

In the compound of formula (I) X and Y independently represent a nitrogen atom or a —CR₉ group, wherein at least one of X and Y represents a nitrogent atom, and R₉ is as defined above. In other words, when X represents a nitrogen atom, Y represents a —CR₉ group; when X represents a —CR₉ group, Y represents a nitrogen atom.

Typically, in the compound of formula (I), R₁ represents a hydrogen atom, a halogen atom, a hydroxy group, a cyano group, a linear or branched C₁-C₆ alkyl group, a C₁-C₄ haloalkyl group, a C₁-C₄ hydroxyalkyl group, a C₁-C₄ alkoxy group, a C₁-C₄ alkoxycarbonyl group, a C₃-C₇ cycloalkyl group, a phenyl group, a pyridyl group, a 6-membered, saturated N-containing heterocyclyl ring, a —C(O)OR₁₃ group, a —C(O)—(CH₂)ₙ—R₁₃ group, a —NR₁₃R₁₄ group, or a —C(O)—(CH₂)ₙ—NR₁₃R₁₄ group, wherein n, R₁₃ and R₁₄ are as defined in claim 1. Preferably R₁ represents a hydrogen atom, a halogen atom, a cyano group, a linear or branched C₁-C₆ alkyl group or a C₃-C₇ cycloalkyl group or a —NR₁₃R₁₄ group wherein R₁₃ and R₁₄ independently represent a hydrogen atom or a linear or branched C₁-C₃ alkyl group. More preferably R₁ represents a hydrogen atom or a —NR₁₃R₁₄ group wherein R₁₃ and R₁₄ independently represent a hydrogen atom or a linear or branched C₁-C₃ alkyl group. Most preferably R₁ represents a hydrogen atom.

Typically, in the compound of formula (I), R₂ represents a hydrogen atom, a halogen atom, a hydroxy group, a cyano group, a linear or branched C₁-C₆ alkyl group, a C₁-C₄ haloalkyl group, a C₁-C₄ alkoxy group, a C₁-C₄ hydroxyalkyl group, a C₁-C₄ alkoxycarbonyl group, a C₃-C₇ cycloalkyl group, a phenyl group, a pyridyl group or a 6 membered, saturated N-containing heterocyclyl ring. Preferably R₂ represents a hydrogen atom, a halogen atom, a cyano group, a linear or branched C₁-C₆ alkyl group or a C₃-C₇ cycloalkyl group. More preferably R₂ represents a hydrogen atom or a halogen atom. Most R₂ preferably represents a hydrogen atom.

Typically, in the compound of formula (I), R₃ represents a hydrogen atom, a halogen atom, a cyano group, a linear or branched C₁-C₆ alkyl group, a C₁-C₄ haloalkyl group, a C₁-C₄ hydroxyalkyl group, a C₁-C₄ alkoxy group, a C₁-C₄ alkoxycarbonyl group, a C₃-C₇ cycloalkyl group, a phenyl group, a 5- to 6-membered heteroaryl group containing at least one heteroatom selected from O, S and N, or a 6 membered, saturated N-containing heterocyclyl ring,
the haloalkyl, hydroxyalkyl, alkoxycarbonyl, cycloalkyl, phenyl, heteroaryl and heterocyclyl groups being unsubstituted or substituted by one or more substituents selected from a halogen atom, a hydroxy group, a cyano group or a linear or branched C₁-C₆ alkyl group, and the alkyl groups being unsubstituted or substituted by one or more cyano groups; or
R₃ represents a —S(O)₂NR₁₃R₁₄ group, a —NR₁₃S(O)₂NR₁₄ group, a —OR₁₃ group, a —C(O)OR₁₃ group, a —NR₁₃1R₁₄ group, a —NC(O)—(CH₂)ₙ—R₁₃ group, a —C(O)—(CH₂)ₙ—NR₁₃R₁₄ group, a —NR₁₃C(O)—(CH₂)ₙ—R₁₄ group or a —NR₁₃C(O)—(CH₂)ₙ—NR₁₄R₁₅ group, wherein n is 0 or 1, and R₁₃, R₁₄, and R₁₅ each independently represent a hydrogen atom, a cyano group, a linear or branched C₁-C₆ alkyl group, a C₁-C₄ haloalkyl group, a C₁-C₄ hydroxyalkyl group, a C₁-C₄ alkoxycarbonyl group, a C₃-C₇ cycloalkyl group, a phenyl group or a 6-membered, saturated N-containing heterocyclyl ring.

Preferably, R₃ represents a hydrogen atom, a halogen atom, a cyano group, a C₁-C₄ haloalkyl group, a C₃-C₄ cycloalkyl group, a phenyl group, a 5- to 6-membered monocyclic heteroaryl group containing 1, 2 or 3 heteroatoms selected from N, O and S,
the phenyl and heteroaryl groups being unsubstituted or substituted with one or more substituents selected from a halogen atom, a hydroxy group, a cyano group or a linear or branched C₁-C₆ alkyl group; or
R₃ represents a —C(O)OR₁₃ group or a —C(O)—(CH₂)ₙ—NR₁₃R₁₄ group wherein R₁₃ and R₁₄ independently represents a hydrogen atom, a cyano group, a linear or branched C₁-C₆ alkyl group, a C₁-C₄ haloalkyl group, a C₁-C₄ hydroxyalkyl group, a C₁-C₄ alkoxycarbonyl group, a C₃-C₇ cycloalkyl group, a phenyl group or a 6 membered, saturated N-containing heterocyclyl ring. More preferably R₃ represents a hydrogen atom, a halogen atom or a cyano group or a —C(O)—(CH₂)ₙ—NR₁₃R₁₄ group wherein n is 0 or 1 and R₁₃ and R₁₄ each independently represent a hydrogen atom or a linear or branched C₁-C₃ alkyl group. Most preferably R₃ represents a halogen atom or a cyano group.

Typically, in the compound of formula (I), R₄ represents a hydrogen atom, a halogen atom, a hydroxy group, a cyano group, a linear or branched C₁-C₆ alkyl group, a C₁-C₄ haloalkyl group, a C₁-C₄ hydroxyalkyl group, a C₁-C₄ alkoxy group, a C₁-C₄ alkoxycarbonyl group, a C₃-C₇ cycloalkyl group, a phenyl group, a pyridyl group or a 6 membered, saturated N-containing heterocyclyl ring. Preferably R₄ represents a hydrogen atom, a halogen atom, a cyano group, a linear or branched C₁-C₆ alkyl group or a C₃-C₄ cycloalkyl group. More preferably R₄ represents a hydrogen atom.

Typically, in the compound of formula (I), R₅ represents a hydrogen atom, or a linear or branched C₁-C₆ alkyl group optionally substituted by one or more substituents selected from a hydroxy group, a cyano group, a linear or branched C₁-C₆ alkyl group, a C₁-C₄ haloalkyl group, a C₁-C₄ hydroxyalkyl group, a C₃-C₇ cycloalkyl group, a phenyl group or a 6 membered, saturated N-containing heterocyclyl ring. Preferably R₅ represents a hydrogen atom, a linear or branched C₁-C₆ alkyl group optionally substituted by a C₃-C₇ cycloalkyl group. More preferably R₅ represents a hydrogen atom or a linear or branched C₁-C₃ alkyl group.

Typically, in the compound of formula (I) R₆ and R₇ each independently represent a hydrogen atom or a linear or branched C₁-C₆ alkyl group unsubstituted or substituted by one or more a C₁-C₂ alkoxy groups. Preferably R₆ and R₇ each independently represent a hydrogen atom or a linear or branched C₁-C₆ alkyl group. More preferably R₆ and R₇ each independently represent a hydrogen atom or a linear or branched C₁-C₃ alkyl group. Most preferably R₆ and R₇ independently represent a hydrogen atom, a methyl group or an ethyl group.

Typically, in the compound of formula (I), R₉ represents a hydrogen atom, a halogen atom, a hydroxy group, a cyano group, a linear or branched C₁-C₆ alkyl group, a C₁-C₄ haloalkyl group, a C₁-C₄ alkoxy group, a C₃-C₇ cycloalkyl group, a —C₁-C₄ alkyl-C₃-C₇ cycloalkyl group, a phenyl group, a 5- to 6-membered monocyclic heteroaryl group containing 1, 2, 3 or 34 heteroatoms selected from N, O and S, a 5- to 7-membered heterocyclyl group containing at least one heteroatom selected from O, S and N,
wherein the cycloalkyl, phenyl, heteroaryl and heterocyclyl groups are unsubstituted or substituted by one or more substituents selected from a halogen atom, a hydroxy group, a cyano group, a linear or branched C₁-C₆ alkyl group, a —S(O)₂R₁₀ group, a —C(O)OR₁₀ group, a —C(O)—(CH₂)—R₁₀ group or a —OR₁₀ group,
   wherein R₁₀ represents a hydrogen atom, a cyano group, linear or branched C₁-C₄ alkyl group, a C₁-C₄ haloalkyl group or a C₃-C₇ cycloalkyl group,
      wherein the cycloalkyl group is unsubstituted or substituted by one or more substituents selected from a halogen atom, a hydroxy group, a cyano group, a linear or branched C₁-C₆ alkyl group or a C₁-C₄ haloalkyl group; or
R₉ represents a —S(O)₂R₁₃ group, a —OR₁₃ group, a —C(O)OR₁₃ group, a —NR₁₃R₁₄ group or a —C(O)—(CH₂)ₙ—NR₁₃R₁₄ group wherein n is 0 or 1 and R₁₃ and R₁₄ each independently represent a hydrogen atom, a linear or branched C₁-C₆ alkyl group, a —(C₁-C₄ alkyl)-(C₁-C₄ alkoxy) group, or a —(C₁-C₄ alkyl)-(5- to 7-membered heterocyclyl group containing at least one heteroatom selected from O, S and N) group; or
in the case that two —CR₉ adjacent groups are present, two adjacents —CR₉ groups and the carbon atoms to which they are bonded optionally form a 5- to 9-membered aryl or heteroaryl group, said heteroaryl group containing at least one heteroatom selected from O, S and N, wherein the aryl and heteroaryl groups are unsubstituted or substituted by one or more substituents selected from a halogen atom or a linear or branched C₁-C₄ alkyl group.

Preferably, R₉ represents a hydrogen atom, a halogen atom, a hydroxy group, a cyano group, a linear or branched C₁-C₆ alkyl group, a C₁-C₄ haloalkyl group, a C₁-C₄ alkoxy group, a C₃-C₇ cycloalkyl group, a —C₁-C₄ alkyl-C₃-C₇ cycloalkyl group, a phenyl group, a 5- to 6-membered monocyclic heteroaryl group containing 1, 2 or 3 heteroatoms selected from N, O and S, a 5- to 7-membered heterocyclyl group containing at least one heteroatom selected from O, S and N,
the alkyl, haloalkyl, alkoxy, cycloalkyl, phenyl, heteroaryl and heterocyclyl groups being unsubstituted or substituted by one or more substituents selected from a halogen atom, a hydroxy group, a cyano group, a linear or branched C₁-C₆ alkyl group, a —C(O)—(CH₂)—R₁₀ group or a —OR₁₀ group,
   wherein R₁₀ represents a hydrogen atom, a cyano group, linear or branched C₁-C₄ alkyl group, a C₁-C₄ haloalkyl group or a C₃-C₇ cycloalkyl group,
      the alkyl, haloalkyl and cycloalkyl groups being unsubstituted or substituted by one or more substituents selected from a halogen atom, a hydroxy group, a cyano group, a linear or branched C₁-C₆ alkyl group or a C₁-C₄ haloalkyl group; or
R₉ represents a —S(O)₂R₁₃ group, a —C(O)OR₁₃ group, a —NR₁₃R₁₄ group or a —C(O)—(CH₂)ₙ—NR₁₃R₁₄ group wherein n is 0 or 1 and R₁₃ and R₁₄ independently represent a hydrogen atom or a linear or branched C₁-C₆ alkyl group; or
in the case that two —CR₉ adjacent groups are present, two adjacents —CR₉ groups and the carbon atoms to which they are bonded optionally form a 5- to 9-membered aryl or heteroaryl group, said heteroaryl group containing at least one heteroatom selected from O, S and N, the aryl and heteroaryl groups being unsubstituted or substituted by one or more substituents selected from a halogen atom or a linear or branched C₁-C₄ alkyl group.

More preferably, R₉ represents a hydrogen atom, a halogen atom, a cyano group, a linear or branched C₁-C₄ alkyl group, a C₁-C₄ haloalkyl group, a C₃-C₇ cycloalkyl group, C₁-C₃ alkyl —C₃-C₇ cycloalkyl group, a 5- to 6-membered monocyclic heteroaryl group containing 1, 2 or 3 nitrogen atoms, a 5- to 7-membered heterocyclyl group containing 1, 2 or 3 nitrogen atoms, the alkyl, cycloalkyl, heteroaryl and heterocyclyl group being unsubstituted or substituted by one or more substituents selected from a cyano group or a —OR₁₀ group wherein R₁₀ represents a hydrogen atom or a linear or branched C₁-C₃ alkyl group, or R₉ represents a —S(O)₂R₁₃ group, a —C(O)OR₁₃ group, a —NR₁₃R₁₄ group or a —C(O)—(CH₂)ₙ—NR₁₃R₁₄ group wherein n is 0 or 1 and R₁₃ and R₁₄ independently represent a hydrogen atom or a linear or branched C₁-C₆ alkyl group.

Typically, in the compound of formula (I) R₈ represents a linear or branched C₁-C₆ alkyl group, a C₁-C₄ haloalkyl group, a —(C₁-C₆ alkyl)-(C₁-C₄ alkoxy) group, a C₃-C₁₀ cycloalkyl group, a phenyl group, a naphthyl group, a 5- to 6-membered monocyclic heteroaryl group containing 1, 2 or 3 heteroatoms selected from N, O and S, a 5- to 7-membered heterocyclyl group containing 1, 2 or 3 heteroatoms selected from N, O and S, a C₃-C₇ heterocycloalkyl ketone group containing 1, 2 or 3 nitrogen atoms, or a bicyclyl group containing a monocyclic C₅-C₉ aryl or heteroaryl group bonded directly to a 5- to 9-membered cycloalkyl or heterocyclyl group, said heteroaryl or heterocyclyl group containing at least one heteroatom selected from O, S and N,
the cycloalkyl, phenyl, naphthyl, heteroaryl, heterocyclyl, heterocycloalkyl ketone and bicyclyl groups are unsubstituted or substituted by one or more substituents selected from a halogen atom; a hydroxy group; a cyano group; a linear or branched C₁-C₆ alkyl group; a C₁-C₄ haloalkyl group; a C₃-C₇ cycloalkyl group; a phenyl group, which phenyl group is unsubstituted or substituted by one or more substituents selected from a halogen atom or a cyano group; a 5- to 6-membered monocyclic heteroaryl group containing 1, 2 or 3 heteroatoms selected from N, O and S, which heteroaryl group is unsubstituted or substituted by one or more substituents selected from a halogen atom or a cyano group; a 6 membered, saturated N-containing heterocyclyl ring; a —S(O)₂R₁₀ group; a —S(O)₂NR₁₀R₁₁ group; a —NR₁₀S(O)₂NR₁₁ group; a —OR₁₀ group; a —C(O)OR₁₀ group; a —C(O)—CH₂—OR₁₀ group; a —C(O)—(CH₂)ₙ—O—(CH₂)ₘR₁₀ group;
a —C(O)—(CH₂)ₙ—O—(CH₂)ₘ—O—R₁₀ group; a —C(O)—(CH₂)ₙ—R₁₀ group; a —NR₁₀R₁₁ group; a —C(O)—(CH₂)ₙ—NR₁₀R₁₁ group; a —R₁₀C(O)—(CH₂)ₙ—NR₁₁R₁₂ group;
a —C(C₁-C₄ alkyl)₂-C(O)—(CH₂)ₙ—NR₁₀R₁₁ group; a —(C₁-C₄ alkyl)-CN group, or
a —(C₁-C₄ alkyl)-C(O)NR′R″ group wherein R′ and R″ are the same or different and are selected from hydrogen atoms and linear or branched C₁-C₄ alkyl groups; wherein each n is 0 or an integer from 1 to 5, and wherein each m is an integer from 1 to 3, and wherein
   R₁₀, R₁₁ and R₁₂ each independently represent a hydrogen atom; a cyano group; a linear or branched C₁-C₆ alkyl group; a C₁-C₄ haloalkyl group; a C₁-C₆ hydroxyalkyl group; a C₃-C₇ cycloalkyl group; a phenyl group; a —(C₁-C₄ hydroxyalkyl)-(phenyl) group; a —C(O)-(phenyl) group; a 5- to 6-membered monocyclic heteroaryl group containing 1, 2, 3 or 4 heteroatoms selected from N, O and S; a 5- to 6-membered monocyclic heteroaryl ketone group containing 1, 2 or 3 nitrogen atoms and 1 or 2 ketone groups; a 5- to 6-membered, heterocyclyl group containing 1, 2 or 3 heteroatoms selected from N, O and S; or a bicyclyl group containing a monocyclic C₅-C₆ aryl or heteroaryl group bonded directly to a 5- to 6-membered cycloalkyl or heterocyclyl group, said heteroaryl or heterocyclyl group containing 1, 2 or 3 heteroatoms selected from N, O and S,
      the cycloalkyl, phenyl, heteroaryl, heteroaryl ketone, heterocyclyl and bicyclyl groups being unsubstituted or substituted by one or more substituents selected from a halogen atom, a hydroxy group, a cyano group, a linear or branched C₁-C₆ alkyl group, a C₁-C₄ haloalkyl group, a C₁-C₄ hydroxyalkyl group, a C₁-C₄ alkoxy group, a phenyl group unsubstituted or substituted by one or more halogen atoms, a 5- to 6-membered monocyclic heteroaryl group containing 1, 2 or 3 nitrogen atoms, said heteroaryl group being unsubstituted or substituted by one or more linear or branched C₁-C₃ alkyl groups, or a C₃-C₇ heterocycloalkyl ketone group containing 1, 2 or 3 nitrogen atoms, said heterocycloalkyl ketone group being unsubstituted or substituted by one or more C₁-C₃ alkyl groups; or
R₈ represents a —S(O)₂NR₁₃R₁₄ group, a —NR₁₃S(O)₂NR₁₄ group, a —OR₁₃ group, a —C(O)OR₁₃ group or a —C(O)—(CH₂)ₙ—NR₁₃R₁₄ group, wherein each n is 0 or 1, and each R₁₃ and R₁₄ independently represent a hydrogen atom; a cyano group; a linear or branched C₁-C₆ alkyl group, which alkyl group is unsubstituted or substituted by one or more phenyl groups; a C₁-C₄ haloalkyl group; a C₁-C₄ hydroxyalkyl group; a C₁-C₄ alkoxycarbonyl group; a C₃-C₇ cycloalkyl group; a phenyl group or a 6 membered, saturated N-containing heterocyclyl ring; or
R₈ together with R₅ and the nitrogen atom to which R₅ is bonded form a 5- to 9-membered, saturated heterocyclyl group, which contains, as heteroatoms, one or two nitrogen atoms and which is substituted by a 5- to 6-membered monocyclic heteroaryl group containing 1, 2 or 3 heteroatoms selected from N, O and S;
a —C(O)—(CH₂)ₙ—R₁₀ group; or a —C(O)—(CH₂)ₙ—NR₁₀R₁₁ group, wherein each n is 0 or 1, and each R₁₀ and R₁₁ independently represent a hydrogen atom, a cyano group, a linear or branched C₁-C₆ alkyl group, a C₁-C₄ haloalkyl group, a C₁-C₄ hydroxyalkyl group, a C₁-C₄ alkoxycarbonyl group, a C₃-C₇ cycloalkyl group, a phenyl group or a 6 membered, saturated N-containing heterocyclyl ring,
provided that when m is zero, R₈ is other than a —S(O)₂NR₁₃R₁₄ group,
a —NR₁₃S(O)₂NR₁₄ group, or a —OR₁₃ group.

Preferably, in the compound of formula (I) R₈ represents a linear or branched C₁-C₆ alkyl group, a C₁-C₄ haloalkyl group, a C₃-C₁₀ cycloalkyl group, a phenyl group, a naphthyl group, a 5- to 6-membered monocyclic heteroaryl group containing 1, 2 or 3 heteroatoms selected from N, O and S, a 5- to 7-membered heterocyclyl group containing 1, 2 or 3 heteroatoms selected from N, O and S, or a bicyclyl group containing a monocyclic C₅-C₉ aryl or heteroaryl group bonded directly to a 5- to 9-membered cycloalkyl or heterocyclyl group, said heteroaryl or heterocyclyl group containing at least one heteroatom selected from O, S and N,
the alkyl, haloalkyl, alkoxy, cycloalkyl, phenyl, naphthyl, heteroaryl, heterocyclyl and bicyclyl groups being unsubstituted or substituted by one or more substituents selected from a halogen atom, a hydroxy group, a cyano group, a linear or branched C₁-C₆ alkyl group, a C₁-C₄ haloalkyl group, a C₃-C₇ cycloalkyl group, a phenyl group unsubstituted or substituted by one or more halogen atoms, a 5- to 6-membered monocyclic heteroaryl group containing 1, 2 or 3 heteroatoms selected from N, O and S, a 6 membered, saturated N-containing heterocyclyl ring, a —S(O)₂R₁₀ group, —S(O)₂NR₁₀R₁₁ group, a —NR₁₀S(O)₂NR₁₁ group, a —OR₁₀ group, a —C(O)OR₁₀ group, a —C(O)—(CH₂)ₙ—R₁₀ group, a —NR₁₀R₁₁ group, a —C(O)—(CH₂)ₙ—NR₁₀R₁₁ group or a —R₁₀C(O)—(CH₂)ₙ—NR₁₁R₁₂ group, a —(C₁-C₄ alkyl)-CN group, or a —(C₁-C₄ alkyl)-C(O)NR′R″ group wherein R′ and R″ are the same or different and are selected from hydrogen atoms and linear or branched C₁-C₄ alkyl groups; wherein n is 0 or 1, and
   R₁₀ and R₁₁ each independently represent a hydrogen atom, a cyano group, a linear or branched C₁-C₆ alkyl group, a C₁-C₄ haloalkyl group, a C₃-C₇ cycloalkyl group, a phenyl group, a 5- to 6-membered monocyclic heteroaryl group containing 1, 2 or 3 heteroatoms selected from N, O and S, a 5- to 6-membered, heterocyclyl group containing 1, 2 or 3 nitrogen atoms, or a bicyclyl group containing a monocyclic C₅-C₆ aryl or heteroaryl group bonded directly to a 5- to 6-membered cycloalkyl or heterocyclyl group, said heteroaryl or heterocyclyl group containing 1, 2 or 3 nitrogen atoms,
      the alkyl, haloalkyl, cycloalkyl, phenyl, heteroaryl, heterocyclyl and bicyclyl groups being unsubstituted or substituted by one or more substituents selected from a halogen atom, a hydroxy group, a cyano group, a linear or branched C₁-C₆ alkyl group, a C₁-C₄ haloalkyl group, a phenyl group unsubstituted or substituted by one or more halogen atoms, a 5- to 6-membered monocyclic heteroaryl group containing 1, 2 or 3 nitrogen atoms, said heteroaryl group being unsubstituted or substituted by one or more linear or branched C₁-C₃ alkyl groups, or a C₃-C₇ heterocycloalkyl ketone group containing 1, 2 or 3 nitrogen atoms, said heterocycloalkyl ketone group being unsubstituted or substituted by one or more C₁-C₃ alkyl groups; or
R₈ represents a —S(O)₂NR₁₃R₁₄ group, a —NR₁₃S(O)₂NR₁₄ group, a —OR₁₃ group, a —C(O)OR₁₃ group or a —C(O)—(CH₂)ₙ—NR₁₃R₁₄ group, wherein n is 0 or 1, and R₁₃ and R₁₄ each independently represent a hydrogen atom, a cyano group, a linear or branched C₁-C₆ alkyl group unsubstituted or substituted by one or more phenyl groups, a C₁-C₄ haloalkyl group, a C₁-C₄ hydroxyalkyl group, a C₁-C₄ alkoxycarbonyl group, a C₃-C₇ cycloalkyl group, a phenyl group or a 6 membered, saturated N-containing heterocyclyl ring; or
R₈ together with R₅ and the nitrogen atom to which R₅ is bonded form a 5- to 9-membered, saturated heterocyclyl group, which contains, as heteroatoms, one or two nitrogen atoms and which is substituted by a —C(O)—(CH₂)ₙ—R₁₀ group or a —C(O)—(CH₂)ₙ—NR₁₀R₁₁ group, wherein n is 0 or 1, and R₁₀ and R₁₁ independently represent a hydrogen atom, a cyano group, a linear or branched C₁-C₆ alkyl group, a C₁-C₄ haloalkyl group, a C₁-C₄ hydroxyalkyl group, a C₁-C₄ alkoxycarbonyl group, a C₃-C₇ cycloalkyl group, a phenyl group or a 6 membered, saturated N-containing heterocyclyl ring,
provided that when m is zero, R₈ is other than a —S(O)₂NR₁₃R₁₄ group,
a —NR₁₃S(O)₂NR₁₄ group, or a —OR₁₃ group.

More preferably, in the compound of formula (I) R₈ represents a linear or branched C₁-C₆ alkyl group, a C₁-C₄ haloalkyl group, a C₁-C₄ alkoxy group, a C₃-C₇ cycloalkyl group, an adamantyl group, a phenyl group, a 5- to 6-membered monocyclic heteroaryl group containing 1, 2 or 3 heteroatoms selected from N, O and S, a 5- to 7-membered heterocyclyl group containing 1, 2 or 3 heteroatoms selected from N, O and S, or a bicyclyl group containing a phenyl group bonded directly to a 5- to 7-membered heterocyclyl group containing at least one heteroatom selected from O, S and N,
the alkyl, haloalkyl, alkoxy, cycloalkyl, adamantyl, phenyl, heteroaryl, heterocyclyl and bicyclyl groups being unsubstituted or substituted by one or more substituents selected from a halogen atom, a hydroxy group, a linear or branched C₁-C₆ alkyl group, a phenyl group, a pyridyl group, a —S(O)₂R₁₀,
a —C(O)OR₁₀, a —C(O)—(CH₂)ₙ—R₁₀, a —NR₁₀R₁₁ group,
a —C(O)—(CH₂)ₙ—NR₁₀R₁₁ group, a —(C₁-C₄ alkyl)-CN group, or
a —(C₁-C₄ alkyl)-C(O)NR′R″ group wherein R′ and R″ are the same or different and are selected from hydrogen atoms and linear or branched C₁-C₄ alkyl groups; wherein n is 0 or 1, and
   R₁₀ and R₁₁ each independently represent a hydrogen atom, a cyano group, a linear or branched C₁-C₆ alkyl group, a C₁-C₄ haloalkyl group, a C₃-C₇ cycloalkyl group, a phenyl group, a 5- to 6-membered monocyclic heteroaryl group containing 1, 2 or 3 heteroatoms selected from N, O and S, a 5- to 6-membered, heterocyclyl group containing 1, 2 or 3 nitrogen atoms, or a bicyclyl group containing a monocyclic C₅-C₆ aryl or heteroaryl group bonded directly to a 5- to 6-membered cycloalkyl or heterocyclyl group, said heteroaryl or heterocyclyl group containing 1, 2 or 3 nitrogen atoms,
      the alkyl, haloalkyl, cycloalkyl, phenyl, heteroaryl, heterocyclyl and bicyclyl groups being unsubstituted or substituted by one or more substituents selected from a halogen atom, a hydroxy group, a cyano group, a linear or branched C₁-C₆ alkyl group, a C₁-C₄ haloalkyl group, a phenyl group unsubstituted or substituted by one or more halogen atoms, a 5- to 6-membered monocyclic heteroaryl group containing 1, 2 or 3 nitrogen atoms, said heteroaryl group being unsubstituted or substituted by one or more linear or branched C₁-C₃ alkyl groups, or a C₃-C₇ heterocycloalkyl ketone group containing 1, 2 or 3 nitrogen atoms, said heterocycloalkyl ketone group being unsubstituted or substituted by one or more linear or branched C₁-C₃ alkyl groups; or
R₈ represents a —OR₁₃ group wherein R₁₃ represents a C₁-C₃ alkyl group unsubstituted or substituted by one or more phenyl groups, or
R₈ together with R₅ and the nitrogen atom to which R₅ is bonded form a 5- to 7-membered, saturated heterocyclyl group, which contains, as heteroatoms, one or two nitrogen atoms and which is substituted by a —C(O)—(CH₂)ₙ—R₁₀ group or
a —C(O)—(CH₂)ₙ—NR₁₀R₁₁ group, wherein n is 0 or 1, and R₁₀ and R₁₁ independently represent a hydrogen atom, a cyano group, a linear or branched C₁-C₃ alkyl group or a C₁-C₄ haloalkyl group,
provided that when m is zero, R₈ is other than an —OR₁₃ group.

In one embodiment, R₈ represents a linear or branched C₁-C₆ alkyl group, a C₁-C₄ haloalkyl group, a C₃-C₉ cycloalkyl group, a phenyl group, a 5- to 6-membered monocyclic heteroaryl group containing 1, 2 or 3 heteroatoms selected from N, O and S, a 5- to 7-membered heterocyclyl group containing 1, 2 or 3 heteroatoms selected from N, O and S, a C₃-C₇ heterocycloalkyl ketone group containing 1, 2 or 3 nitrogen atoms, a bicyclyl group containing a phenyl group bonded directly to a 5- to 7-membered heterocyclyl group containing at least one heteroatom selected from O, S and N, or a bicyclyl group containing a pyridinyl group bonded directly to a C₃-C₇ cycloalkyl group, or R₈ represents a —(CH₂)ₙOR group wherein n is 0, 1 or 2 and R represents a linear or branched C₁-C₆ alkyl group or a C₁-C₄ haloalkyl group; in particular, R₈ may represent a 6-membered heterocyclyl group containing 1, 2 or 3 heteroatoms selected from N, O and S;
wherein the haloalkyl, cycloalkyl, adamantyl, phenyl, heteroaryl, heterocyclyl, heterocycloalkyl ketone and bicyclyl groups are unsubstituted or substituted are unsubstituted or substituted by one or more substituents selected from Ra; and the alkyl group is unsubstituted or substituted by one or more substituents selected from Rb, wherein Ra and Rb are as defined above.

More preferably, when R₈ is an alkyl or haloalkyl group, it is an unsubstituted alkyl or haloalkyl group; when R₈ is a cycloalkyl (cyclohexyl and adamantyl groups being preferred) or phenyl group, it is unsubstituted or substituted by one or more substituents selected from a halogen atom, a cyano group, a hydroxy group, a linear or branched C₁-C₆ alkyl group, a C₁-C₄ haloalkyl group, or a —(C₁-C₄ alkyl)-CN group; and when R₈ is a heteroaryl or heterocyclyl group it is unsubstituted or substituted with one or more substituents selected from Ra, wherein Ra is as defined above.

Preferably, when R₈ is a phenyl group, it is unsubstituted or substituted by one or more substituents selected from a halogen atom, a cyano group, a hydroxy group or a linear or branched C₁-C₃ alkyl group. It is also preferred that when R₈ is a phenyl group m is an integer from 1 to 3, preferably 1.

Preferably, when R₈ is a heteroaryl group, it is a 5- to 6-membered heteroaryl group containing one or two nitrogen atoms. Pyridinyl is preferred. Preferably, when R₈ is a heteroaryl group it is unsubstituted or substituted with one or more halogen atoms. It is also preferred that when R₈ is a heteroaryl group m is an integer from 1 to 3, preferably 1.

When R₈ is a heterocyclyl group it is preferably a 5- or 6-membered heterocyclyl group, containing one or two heteroatoms selected from N and O, more preferably containing one or two nitrogen atoms. Preferred examples are piperidinyl, pyrrolidinyl and tetrahydropyranyl. Piperidinyl is preferred. Preferably, the heterocyclyl group is linked to the rest of the molecule via a ring carbon atom, in other words it is linked to the group —Z—(CR₆R₇)ₘ— via a ring carbon atom. Substituents on a piperidinyl group may be present on any ring atom but are preferably present on the nitrogen atom. Preferably, at least one substituent is present on the ring nitrogen atom.

Most preferably, R₈ represents a linear or branched C₁-C₆ alkyl group, a —(C₁-C₅ alkyl)-(C₁-C₂ alkoxy) group, a ciclohexyl group, an adamantyl group, a pyridinyl group, a 5,6,7,8-tetrahydroquinolinyl group, a tetrahydropyranyl group or a chromanyl group, which ciclohexyl, adamantyl, pyridinyl, tetrahydroquinolinyl, tetrahydropyranyl and chromanyl groups are unsubstituted or substituted by one ore more substituents selected from a halogen atom, a hydroxy group, a linear or branched C₁-C₃ alkyl group or a C₁-C₄ haloalkyl group; or R₈ is a piperidinyl group, a piperazinyl group, a pyrrolidinyl group or a pyrrolidin-2-one group, which piperidinyl, piperazinyl, pyrrolidinyl and pyrrolidin-2-one groups are unsubstituted or substituted by one ore more substituents selected from a linear or branched C₁-C₃ alkyl group, a phenyl group, a pyridinyl group, a triazololyl group, a thiazolyl group, a —S(O)₂₋(CH₂)₀₋₁R₁₀ group,
a —C(O)—(CH₂)₀₋₁OR₁₀ group, a —C(O)—(CH₂)₀₋₁R₁₀ group,
a —C(O)—(CH₂)ₙ—NR₁₀R₁₁ group, a —(C₁-C₄ alkyl)-CN group, or
a —(C₁-C₄ alkyl)-C(O)NR₁₀R₁₁, wherein the phenyl, pyridinyl, triazololyl and thiazolyl groups are unsubstituted or substituted by one or more substituents selected from a halogen atom, a hydroxy group, a cyano group, a linear or branched C₁-C₃ alkyl group or a C₁-C₄ haloalkyl group, and wherein
   R₁₁ represents a hydrogen atom or a linear or branched C₁-C₄ alkyl group, and
   R₁₀ represents a hydrogen atom; a cyano group; a linear or branched C₁-C₅ alkyl group; a C₁-C₄ haloalkyl group; a C₁-C₅ hydroxyalkyl group;
   a —(C₁-C₄ alkyl)(C₁-C₂ alkoxy) group; a C₃-C₆ cycloalkyl group, which cycloalkyl group is unsubstituted or substituted by one or more substituents selected from a halogen atom, a hydroxy group, a cyano group, a linear or branched C₁-C₃ alkyl group, a —(C₁-C₄ alkyl)-CN group or a phenyl group; a pyridinyl group, which pyridinyl group is unsubstituted or substituted by one or more substituents selected from a halogen atom or a cyano group; a phenyl group, which phenyl group is unsubstituted or substituted by one or more substituents selected from a halogen atom, a cyano group, a linear or branched C₁-C₃ alkyl group or a C₁-C₄ hydroxyalkyl group; a pyrrolidinyl group, which pyrrolidinyl group is unsubstituted or substituted by one or more substituents selected from a halogen atom or a cyano group; a pyrimidinyl group, which pyrimidinyl group is unsubstituted or substituted by one or more substituents selected from a halogen atom, a hydroxy group or a linear or branched C₁-C₃ alkyl group; a thiazolyl group, which thiazolyl group is unsubstituted or substituted by one or more substituents selected from a halogen atom, a cyano group or a linear or branched C₁-C₃ alkyl group; an imidazolyl group, a —(C₁-C₅ alkyl)-(phenyl) group, wherein the phenyl group of the —(C₁-C₅ alkyl)-(phenyl) group is unsubstituted or substituted by one or more substituents selected from a halogen atom or a C₁-C₂ alcoxy group; a —(C₁-C₅ hydroxyalkyl)-(phenyl) group, wherein the phenyl group of the —(C₁-C₅ hydroxyalkyl)-(phenyl) group is unsubstituted or substituted by one or more substituents selected from a halogen atom or a C₁-C₂ alcoxy group; a 1-thia-2,3-diazolyl group, which 1-thia-2,3-diazolyl group is unsubstituted or substituted by one or more linear or branched C₁-C₃ alkyl groups; a benzimidazolyl group, which benzimidazolyl group is unsubstituted or substituted by one or more linear or branched C₁-C₃ alkyl groups; a chromanyl group; a 2,2-dimethyl-1,3-dioxolane group; an imidazolyl group; an indolyl group; a tetrahydropyranyl group; a tetrazolyl group; a triazolyl group; a pyrazolyl group or a pyrimidine-2,4(1H,3H)-dione group.

In a particularly preferred embodiment, in the compound of formula (I)
m is 0 or 1;
Z is NR_{S};
X is a nitrogen atom and Y is a group —CR₉, or Y is a nitrogen atom and X is a group —CR₉;
R₁ represents a hydrogen atom, a halogen atom, a cyano group, a linear or branched C₁-C₆ alkyl group or a C₃-C₄ cycloalkyl group or a —NR₁₃N₁₄ group wherein R₁₃ and R₁₄ each independently represent a hydrogen atom or a linear or branched C₁-C₃ alkyl group;
R₂ represents a hydrogen atom, a halogen atom, a cyano group, a linear or branched C₁-C₆ alkyl group or a C₃-C₄ cycloalkyl group;
R₃ represents a hydrogen atom, a halogen atom, a cyano group, a C₃-C₄ cycloalkyl group, a 5- to 6-membered monocyclic aryl or heteroaryl group, the heteroaryl group containing 1, 2 or 3 heteroatoms selected from N, O and S, the aryl and heteroaryl groups being unsubstituted or substituted by one or more substituents selected from a halogen atom, a hydroxy group, a cyano group or a linear or branched C₁-C₆ alkyl group, or
R₃ represents a —C(O)OR₁₃ group, a —C(O)—NR₁₃R₁₄ group or a —NR₁₃C(O)R₁₄ group wherein R₁₃ and R₁₄ each independently represent a hydrogen atom, a linear or branched C₁-C₆ alkyl group, a C₁-C₄ haloalkyl group, a C₁-C₄ hydroxyalkyl group, a C₁-C₄ alkoxycarbonyl group, a C₃-C₇ cycloalkyl group, a phenyl group or a 6 membered, saturated N-containing heterocyclyl ring;
R₄ represents a hydrogen atom, a halogen atom, a cyano group, a linear or branched C₁-C₆ alkyl group or a C₃-C₄ cycloalkyl group;
R₅ represents a hydrogen atom, a linear or branched C₁-C₃ alkyl group or a C₁-C₃ alkyl —C₃-C₇ cycloalkyl group;
R₆ and R₇ each independently represent a hydrogen atom or a linear or branched C₁-C₃ alkyl group unsubstituted or substituted by a C₁-C₂ alkoxy group;
R₈ represents a linear or branched C₁-C₆ alkyl group, a C₁-C₄ haloalkyl group, a C₃-C₇ cycloalkyl group, an adamantyl group, a phenyl group, a 5- to 6-membered monocyclic heteroaryl group containing 1, 2 or 3 heteroatoms selected from N, O and S, a 5- to 7-membered heterocyclyl group containing 1, 2 or 3 heteroatoms selected from N, O and S, a C₃-C₇ heterocycloalkyl ketone group containing 1, 2 or 3 nitrogen atoms, a bicyclyl group containing a phenyl group bonded directly to a 5- to 7-membered heterocyclyl group containing at least one heteroatom selected from O, S and N, or a bicyclyl group containing a pyridinyl group bonded directly to a C₃-C₇ cycloalkyl group, wherein
   the cycloalkyl, adamantyl, phenyl, heteroaryl, heterocyclyl, heterocycloalkyl ketone and bicyclyl groups are unsubstituted or substituted by one or more substituents selected from a halogen atom; a hydroxy group; a linear or branched C₁-C₆ alkyl group; a phenyl group, which phenyl group is unsubstituted or substituted by one or more substituents selected from a halogen atom or a cyano group; a 5- to 6-membered monocyclic heteroaryl group containing 1, 2 or 3 heteroatoms selected from N, O and S, which heteroaryl group is unsubstituted or substituted by one or more substituents selected from a halogen atom or a cyano group, a —S(O)₂R₁₀ group, a —C(O)OR₁₀, a —C(O)—CH₂—OR₁₀ group, a —C(O)—(CH₂)ₙ—O—(CH₂)ₘR₁₀ group, a —C(O)—(CH₂)ₙ—O—(CH₂)ₘ—O—R₁₀ group, a —C(O)—(CH₂)ₙ—R₁₀, a —NR₁₀R₁₁ group, a —C(O)—(CH₂)ₙ—NR₁₀R₁₁ group, a —C(C₁-C₄ alkyl)₂-C(O)—(CH₂)ₙ—NR₁₀R₁₁ group, a —(C₁-C₄ alkyl)-CN group, or a —(C₁-C₄ alkyl)-C(O)NR′R″ group wherein R′ and R″ are the same or different and are selected from hydrogen atoms and linear or branched C₁-C₄ alkyl groups; wherein each n is 0 or an integer from 1 to 5, and wherein m is an integer from 1 to 3, and wherein
      R₁₀ and R₁₁ each independently represent a hydrogen atom, a cyano group, a linear or branched C₁-C₆ alkyl group, a C₁-C₄ haloalkyl group, a C₁-C₆ hydroxyalkyl group, a C₃-C₇ cycloalkyl group, a phenyl group, a —(C₁-C₄ hydroxyalkyl)-(phenyl) group, a —C(O)-(phenyl) group, a 5- to 6-membered monocyclic heteroaryl group containing 1, 2, 3 or 4 heteroatoms selected from N, O and S, a 9- to 10-membered bicyclic heteroaryl group containing 1, 2 or 3 nitrogen atoms, a 5- to 6-membered monocyclic heteroaryl ketone group containing 1, 2 or 3 nitrogen atoms and 1 or 2 ketone groups, a 5- to 6-membered, heterocyclyl group containing 1, 2 or 3 heteroatoms selected from N, O and S, or a bicyclyl group containing a monocyclic C₅-C₆ aryl or heteroaryl group bonded directly to a 5- to 6-membered cycloalkyl or heterocyclyl group, said heteroaryl or heterocyclyl group containing 1, 2 or 3 heteroatoms selected from N, O and S,
         the cycloalkyl, phenyl, heteroaryl, heterocyclyl and bicyclyl groups being unsubstituted or substituted by one or more substituents selected from a halogen atom; a hydroxy group; a cyano group; a linear or branched C₁-C₆ alkyl group; a C₁-C₄ haloalkyl group; a C₁-C₄ hydroxyalkyl group; a C₁-C₄ alkoxy group; a phenyl group, which phenyl group is unsubstituted or substituted by one or more halogen atoms; a 5- to 6-membered monocyclic heteroaryl group containing 1, 2 or 3 nitrogen atoms, which heteroaryl group is unsubstituted or substituted by one or more linear or branched C₁-C₃ alkyl groups; or a C₃-C₇ heterocycloalkyl ketone group containing 1, 2 or 3 nitrogen atoms, which heterocycloalkyl ketone group is unsubstituted or substituted by one or more linear or branched C₁-C₃ alkyl groups; or
R₈ represents a —OR₁₃ group wherein R₁₃ represents a C₁-C₃ alkyl group unsubstituted or substituted by one or more phenyl groups, or
R₈ together with R₅ and the nitrogen atom to which R₅ is bonded form a 5- to 7-membered, saturated heterocyclyl group, which contains, as heteroatoms, one or two nitrogen atoms and which, is substituted by a pyridinyl group, a —C(O)—(CH₂)ₙ—R₁₀ group or a —C(O)—(CH₂)ₙ—NR₁₀R₁₁ group, wherein each n is 0 or 1, and wherein each R₁₀ and R₁₁ independently represent a hydrogen atom, a cyano group, a linear or branched C₁-C₃ alkyl group or a C₁-C₄ haloalkyl group,
R₉ represents a hydrogen atom, a halogen atom, a cyano group, a linear or branched C₁-C₄ alkyl group, a C₁-C₄ haloalkyl group, a C₃-C₇ cycloalkyl group, a 5- to 6-membered monocyclic heteroaryl group containing 1, 2, 3 or 34 heteroatoms selected from N, O and S, or a 5- to 7-membered heterocyclyl group containing 1, 2 or 3 heteroatoms selected from N, O and S, wherein the alkyl, cycloalkyl, heteroaryl, and heterocyclyl groups are unsubstituted or substituted by one or more substituents selected from a halogen atom, a cyano group, a hydroxy group, a linear or branched C₁-C₆ alkyl group, a —S(O)₂R₁₀ group, a —C(O)OR₁₀ group, a —C(O)—(CH₂)—R₁₀ group or a —OR₁₀ group, wherein R₁₀ represents a hydrogen atom, a cyano group or linear or branched C₁-C₃ alkyl group, or
R₉ represents a —S(O)₂R₁₃ group, a —OR₁₃ group, a —C(O)OR₁₃ group, a —NR₁₃R₁₄ group or a —C(O)—(CH₂)ₙ—NR₁₃R₁₄ group wherein n is 0 or 1 and R₁₃ and R₁₄ each independently represent a hydrogen atom, a linear or branched C₁-C₆ alkyl group, a —(C₁-C₄ alkyl)-(C₁-C₄ alkoxy) group, or a —(C₁-C₄ alkyl)-(5- to 7-membered heterocyclyl group containing at least one heteroatom selected from O, S and N) group; or
in the case that two —CR₉ adjacent groups are present, both —CR₉ groups and the carbon atoms to which they are bonded optionally form a 5- to 6-membered aryl group, wherein the aryl group is unsubstituted or substituted by one or more substituents selected from a halogen atom or a linear or branched C₁-C₄ alkyl group,
provided that when m is zero, R₉ is other than an —OR₁₃ group.

In another particularly preferred embodiment, in the compound of formula (I)
m is 0 or 1;
Z is NR₅;
X is a nitrogen atom and Y is a group —CR₉, or Y is a nitrogen atom and X is a group —CR₉;
R₁ represents a hydrogen atom, a halogen atom, a cyano group, a linear or branched C₁-C₆ alkyl group or a C₃-C₄ cycloalkyl group or a —NR₁₃N₁₄ group wherein R₁₃ and R₁₄ each independently represent a hydrogen atom or a linear or branched C₁-C₃ alkyl group;
R₂ represents a hydrogen atom, a halogen atom, a cyano group, a linear or branched C₁-C₆ alkyl group or a C₃-C₄ cycloalkyl group;
R₃ represents a hydrogen atom, a halogen atom, a cyano group, a C₃-C₄ cycloalkyl group, a 5- to 6-membered monocyclic aryl or heteroaryl group, the heteroaryl group containing 1, 2 or 3 heteroatoms selected from N, O and S, the aryl and heteroaryl groups being unsubstituted or substituted by one or more substituents selected from a halogen atom, a hydroxy group, a cyano group or a linear or branched C₁-C₆ alkyl group, or
R₃ represents a —C(O)OR₁₃ group, a —C(O)—NR₁₃R₁₄ group or a —NR₁₃C(O)R₁₄ group wherein R₁₃ and R₁₄ each independently represent a hydrogen atom, a linear or branched C₁-C₆ alkyl group, a C₁-C₄ haloalkyl group, a C₁-C₄ hydroxyalkyl group, a C₁-C₄ alkoxycarbonyl group, a C₃-C₇ cycloalkyl group, a phenyl group or a 6 membered, saturated N-containing heterocyclyl ring;
R₄ represents a hydrogen atom, a halogen atom, a cyano group, a linear or branched C₁-C₆ alkyl group or a C₃-C₄ cycloalkyl group;
R₅ represents a hydrogen atom, a linear or branched C₁-C₃ alkyl group or a C₁-C₃ alkyl —C₃-C₇ cycloalkyl group;
R₆ and R₇ each independently represent a hydrogen atom or a linear or branched C₁-C₃ alkyl group;
R₈ represents a linear or branched C₁-C₆ alkyl group, a C₁-C₄ haloalkyl group, a C₁-C₄ alkoxy group, a C₃-C₇ cycloalkyl group, an adamantyl group, a phenyl group, a 5- to 6-membered monocyclic heteroaryl group containing 1, 2 or 3 heteroatoms selected from N, O and S, a 5- to 7-membered heterocyclyl group containing 1, 2 or 3 heteroatoms selected from N, O and S, or a bicyclyl group containing a phenyl group bonded directly to a 5- to 7-membered heterocyclyl group containing at least one heteroatom selected from O, S and N, the alkyl, haloalkyl, alkoxy, cycloalkyl, adamantyl, phenyl, heteroaryl, heterocyclyl and bicyclyl groups being unsubstituted or substituted by one or more substituents selected from
   a halogen atom, a hydroxy group, a linear or branched C₁-C₆ alkyl group, a phenyl group, a pyridyl group, a —S(O)₂R₁₀, a —C(O)OR₁₀, a —C(O)—(CH₂)ₙ—R₁₀, a —NR₁₀R₁₁ group, a —C(O)—(CH₂)ₙ—NR₁₀R₁₁ group, a —(C₁-C₄ alkyl)-CN group, or a —(C₁-C₄ alkyl)-C(O)NR′R″ group wherein R′ and R″ are the same or different and are selected from hydrogen atoms and linear or branched C₁-C₄ alkyl groups; wherein n is 0 or 1, and
      R₁₀ and R₁₁ each independently represent a hydrogen atom, a cyano group, a linear or branched C₁-C₆ alkyl group, a C₁-C₄ haloalkyl group, a C₃-C₇ cycloalkyl group, a phenyl group, a 5- to 6-membered monocyclic heteroaryl group containing 1, 2 or 3 heteroatoms selected from N, O and S, a 5- to 6-membered heterocyclyl group containing 1, 2 or 3 nitrogen atoms, or a bicyclyl group containing a monocyclic C₅-C₆ aryl or heteroaryl group bonded directly to a 5- to 6-membered cycloalkyl or heterocyclyl group, said heteroaryl or heterocyclyl group containing 1, 2 or 3 nitrogen atoms,
         the alkyl, haloalkyl, cycloalkyl, phenyl, heteroaryl, heterocyclyl and bicyclyl groups being unsubstituted or substituted by one or more substituents selected from a halogen atom, a hydroxy group, a cyano group, a linear or branched C₁-C₆ alkyl group, a C₁-C₄ haloalkyl group, a phenyl group unsubstituted or substituted by one or more halogen atoms, a 5- to 6-membered monocyclic heteroaryl group containing 1, 2 or 3 nitrogen atoms, said heteroaryl group unsubstituted or substituted by one or more linear or branched C₁-C₃ alkyl groups, or a C₃-C₇ heterocycloalkyl ketone group containing 1, 2 or 3 nitrogen atoms, said heterocycloalkyl ketone group being unsubstituted or substituted by one or more linear or branched C₁-C₃ alkyl groups, or
R₈ represents a —OR₁₃ group wherein R₁₃ represents a C₁-C₃ alkyl group unsubstituted or substituted by one or more phenyl groups, or
R₈ together with R₅ and the nitrogen atom to which R₅ is bonded form a 5- to 7-membered, saturated heterocyclyl group, which contains, as heteroatoms, one or two nitrogen atoms and which is substituted by a —C(O)—(CH₂)ₙ—R₁₀ group or a —C(O)—(CH₂)ₙ—NR₁₀R₁₁ group, wherein n is 0 or 1, and R₁₀ and R₁₁ independently represent a hydrogen atom, a cyano group, a linear or branched C₁-C₃ alkyl group or a C₁-C₄ haloalkyl group;
R₉ represents a hydrogen atom, a halogen atom, a cyano group, a linear or branched C₁-C₄ alkyl group, a C₁-C₄ haloalkyl group, a C₃-C₇ cycloalkyl group, a —C₁-C₃ alkyl-C₃-C₇ cycloalkyl group, a 5- to 6-membered monocyclic heteroaryl group containing 1, 2 or 3 nitrogen atoms, a 5- to 7-membered heterocyclyl group containing 1, 2 or 3 nitrogen atoms, the alkyl, cycloalkyl, heteroaryl and heterocyclyl groups being unsubstituted or substituted by one or more substituents selected from a cyano group or a —OR₁₀ group, wherein R₁₀ represents a linear or branched C₁-C₃ alkyl group, or
R₉ represents a —S(O)₂R₁₃ group, a —C(O)OR₁₃ group or a —NR₁₃R₁₄ group wherein R₁₃ and R₁₄ each independently represent a hydrogen atom or a linear or branched C₁-C₄ alkyl group,
or in the case that two —CR₉ adjacent groups are present, both —CR₉ groups and the carbon atoms to which they are bonded optionally form a 5- to 9-membered aryl group, the aryl group being unsubstituted or substituted by one or more substituents selected from a halogen atom or a linear or branched C₁-C₄ alkyl group,
provided that when m is zero, R₅ is other than an —OR₁₃ group.

In a further particularly preferred embodiment, in the compound of formula (I)
m is 0 or 1;
Z is NR₅;
X is a nitrogen atom and Y is a group —CR₉, or Y is a nitrogen atom and X is a group —CR₉;
R₁ represents a hydrogen atom;
R₂ represents a hydrogen atom;
R₃ represents a hydrogen atom, a halogen atom, a cyano group, a C₁-C₄ haloalkyl group, a cyclopropyl group, a phenyl group unsubstituted or substituted by one or more halogen atoms, a 5- to 6-membered monocyclic heteroaryl group containing 1, 2 or 3 heteroatoms selected from N and O, or
R₃ represents a —C(O)OR₁₃ group, a —C(O)—NR₁₃R₁₄ group or a —NR₁₃C(O)R₁₄ group wherein R₁₃ and R₁₄ independently represent a hydrogen atom or a methyl group;
R₄ represents a hydrogen atom;
R₅ represents a hydrogen atom, a methyl group, an ethyl group or a —C₁-C₃ alkyl-C₃-C₄ cycloalkyl group;
R₆ and R₇ each independently represent a hydrogen atom or a methyl group;
R₈ represents a linear or branched C₁-C₆ alkyl group, a C₁-C₄ haloalkyl group, a C₁-C₄ alkoxy group, a C₃-C₇ cycloalkyl group, an adamantyl group, a phenyl group, a 5- to 6-membered monocyclic heteroaryl group containing 1, 2 or 3 heteroatoms selected from N, O and S, a 5- to 7-membered heterocyclyl group containing 1, 2 or 3 heteroatoms selected from N, O and S, or a bicyclyl group containing a phenyl group bonded directly to a 5- to 7-membered heterocyclyl group containing at least one heteroatom selected from O, S and N,
   the alkyl, haloalkyl, alkoxy, cycloalkyl, adamantyl, phenyl, heteroaryl, heterocyclyl and bicyclyl groups being unsubstituted or substituted by one or more substituents selected from a halogen atom, a hydroxy group, a linear or branched C₁-C₆ alkyl group, a phenyl group, a pyridyl group, a —S(O)₂R₁₀, a —C(O)OR₁₀, a —C(O)—(CH₂)ₙ—R₁₀, a —NR₁₀R₁₁ a —C(O)—(CH₂)ₙ—NR₁₀R₁₁ group, a —(C₁-C₄ alkyl)-CN group, or a —(C₁-C₄ alkyl)-C(O)NR′R″ group wherein R′ and R″ are the same or different and are selected from hydrogen atoms and linear or branched C₁-C₄ alkyl groups; wherein n is 0 or 1, and
      R₁₀ and R₁₁, independently represent a hydrogen atom, a cyano group, a linear or branched C₁-C₆ alkyl group, a C₁-C₄ haloalkyl group, a C₃-C₇ cycloalkyl group, a phenyl group, a 5- to 6-membered monocyclic heteroaryl group containing 1, 2 or 3 heteroatoms selected from N, O and S, a 5- to 6-membered, heterocyclyl group containing 1, 2 or 3 nitrogen atoms, or a bicyclyl group containing a monocyclic C₅-C₆ aryl or heteroaryl group bonded directly to a 5- to 6-membered cycloalkyl or heterocyclyl group, said heteroaryl or heterocyclyl group containing 1, 2 or 3 nitrogen atoms,
         the alkyl, haloalkyl, cycloalkyl, phenyl, heteroaryl, heterocyclyl and bicyclyl groups being unsubstituted or substituted by one or more substituents selected from a halogen atom, a hydroxy group, a cyano group, a linear or branched C₁-C₆ alkyl group, a C₁-C₄ haloalkyl group, a phenyl group unsubstituted or substituted by one or more halogen atoms, a 5- to 6-membered monocyclic heteroaryl group containing 1, 2 or 3 nitrogen atoms said heteroaryl group being unsubstituted or substituted by one or more linear or branched C₁-C₃ alkyl groups, or a C₃-C₇ heterocycloalkyl ketone group containing 1, 2 or 3 nitrogen atoms, said heterocycloalkyl ketone group being unsubstituted or substituted by one or more linear or branched C₁-C₃ alkyl groups, or
R₈ represents a —OR₁₃ group wherein R₁₃ represents a C₁-C₃ alkyl group unsubstituted or substituted by one or more phenyl groups, or
R₈ together with the R₅ and the nitrogen atom to which R₅ is bonded form a 5- to 7-membered, saturated heterocyclyl group, which contains, as heteroatoms, one or two nitrogen atoms and which is substituted by a —C(O)—(CH₂)ₙ—R₁₀ group or a —C(O)—(CH₂)ₙ—NR₁₀R₁₁ group, wherein n is 0 or 1, and R₁₀ and R₁₁ independently represent a hydrogen atom, a cyano group, a linear or branched C₁-C₃ alkyl group or a C₁-C₄ haloalkyl group;
R₉ represents a hydrogen atom, a halogen atom, a cyano group, a linear or branched C₁-C₄ alkyl group, a C₃-C₇ cycloalkyl group, a 5- to 6-membered monocyclic heteroaryl group containing 1, 2 or 3 nitrogen atoms, a 5- to 7-membered heterocyclyl group containing 1, 2 or 3 nitrogen atoms, the alkyl, cycloalkyl, heteroaryl, and heterocyclyl groups being unsubstituted or substituted by one or more substituents selected from a cyano group or a —OR₁₀ group wherein R₁₀ represents a linear or branched C₁-C₃ alkyl group, or
R₉ represents a —S(O)₂R₁₃ group, a —C(O)OR₁₃ group or a —NR₁₃R₁₄ group, wherein R₁₃ and R₁₄ each independently represent a hydrogen atom or a linear or branched C₁-C₄ alkyl group,
or in the case that two —CR₉ adjacent groups are present, both —CR₉ groups and the carbon atoms to which they are bonded optionally form a 5- to 6-membered aryl group, the aryl group being unsubstituted or substituted by one or more substituents selected from a halogen atom or a linear or branched C₁-C₄ alkyl group,
provided that when m is zero, R₈ is other than an —OR₁₃ group.

In a particularly preferred embodiment, the compound of the invention is of formula (I″),

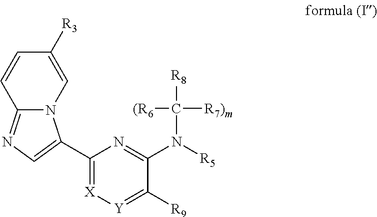

wherein X is a nitrogen atom and Y is a —CR₉ group, or Y is a nitrogen atom and X is a CR₉ group;
R₃ is a hydrogen atom, a halogen atom, a cyano group, a —C(O)OR′ group, a C₃-C₄ cycloalkyl group, a pyridine group, a pyrazole group, or a phenyl group, which phenyl group is unsubstituted or substituted with a halogen atom, wherein R′ is a hydrogen atom or a linear or branched C₁-C₄ alkyl group;
R₅ is a hydrogen atom, a methyl group, an ethyl group, a —(CH₂)-cyclopropyl group; or
R₅ together with R₈ and the nitrogen atom to which R₅ is bonded form:
   a 1,4-diazepane-1-yl group, which 1,4-diazepane-1-yl group is unsubstituted or substituted by a —C(O)CH₂CN group, or
   a pyrrolidinyl group, which pyrrolidinyl group is unsubstituted or substituted by a pyridinyl group or a —C(O)N(CH₂CH₃)₂ group;
R₆ and R₇ each independently represent a hydrogen atom or a linear or branched C₁-C₃ alkyl group unsubstituted or substituted by a C₁-C₂ alkoxy group;
R₈ together with R₅ and the nitrogen atom to which R₅ is bonded form said 1,4-diazepane-1-yl group or pyrrolidinyl group; or R₈ is a hydrogen atom, a linear or branched C₁-C₅ alkyl group, a —(C₁-C₅ alkyl)-(C₁-C₂ alkoxy) group, a cyclohexyl group, an adamantyl group, a phenyl group, a pyridinyl group, a 5,6,7,8-tetrahydroquinolinyl group, a tetrahydropyranyl group or a chromanyl group, which cyclohexyl, adamantyl, phenyl, pyridinyl, tetrahydroquinolinyl, tetrahydropyranyl and chromanyl groups are unsubstituted or substituted by one ore more substituents selected from a halogen atom, a hydroxy group, a —CH₂CN group, a linear or branched C₁-C₃ alkyl group or a C₁-C₄ haloalkyl group; or R₈ is a piperidinyl group, a piperazinyl group, a pyrrolidinyl group or a pyrrolidin-2-one group, which piperidinyl, piperazinyl, pyrrolidinyl and pyrrolidin-2-one groups are unsubstituted or substituted by one ore more substituents selected from a linear or branched C₁-C₃ alkyl group, a phenyl group, a pyridinyl group,
a triazololyl group, a thiazolyl group,
a —S(O)₂₋(CH₂)ₙR₁₀ group, a —C(O)—(CH₂)ₙOR₁₀ group, a —C(O)—(CH₂)ₙR₁₀ group,
a —C(O)—(CH₂)ₙ—N(R₁₀)—(CH₂)ₙR₁₁ group, a —(C₁-C₄ alkyl)-CN group, or
a —(C₁-C₄ alkyl)-C(O)NR₁₀R₁₁, wherein n is 0 or 1, and wherein the phenyl, pyridinyl, triazololyl and thiazolyl groups are unsubstituted or substituted by one or more substituents selected from a halogen atom, a hydroxy group, a cyano group, a linear or branched C₁-C₃ alkyl group or a C₁-C₄ haloalkyl group, and wherein
   R₁₁ represents a hydrogen atom or a linear or branched C₁-C₄ alkyl group, and
   R₁₀ represents a hydrogen atom; a cyano group; a linear or branched C₁-C₅ alkyl group; a C₁-C₄ haloalkyl group; a C₁-C₅ hydroxyalkyl group;
   a —(C₁-C₄ alkyl)(C₁-C₂ alkoxy) group; a C₃-C₆ cycloalkyl group, which cycloalkyl group is unsubstituted or substituted by one or more substituents selected from a halogen atom, a hydroxy group, a cyano group, a linear or branched C₁-C₃ alkyl group, a C₁-C₃ haloalkyl group, a —(C₁-C₄ alkyl)-CN group or a phenyl group; a pyridinyl group, which pyridinyl group is unsubstituted or substituted by one or more substituents selected from a halogen atom or a cyano group; a phenyl group, which phenyl group is unsubstituted or substituted by one or more substituents selected from a halogen atom, a cyano group, a linear or branched C₁-C₃ alkyl group or a C₁-C₄ hydroxyalkyl group; a pyrrolidinyl group, which pyrrolidinyl group is unsubstituted or substituted by one or more substituents selected from a halogen atom or a cyano group; a pyrimidinyl group, which pyrimidinyl group is unsubstituted or substituted by one or more substituents selected from a halogen atom, a hydroxy group or a linear or branched C₁-C₃ alkyl group; a thiazolyl group, which thiazolyl group is unsubstituted or substituted by one or more substituents selected from a halogen atom, a cyano group or a linear or branched C₁-C₃ alkyl group; an imidazolyl group, a —(C₁-C₅ alkyl)-(phenyl) group, wherein the phenyl group of the —(C₁-C₅ alkyl)-(phenyl) group is unsubstituted or substituted by one or more substituents selected from a halogen atom or a
   C₁-C₂ alkoxy group; a —(C₁-C₅ hydroxyalkyl)-(phenyl) group, wherein the phenyl group of the —(C₁-C₅ hydroxyalkyl)-(phenyl) group is unsubstituted or substituted by one or more substituents selected from a halogen atom or a C₁-C₂ alkoxy group; a 1-thia-2,3-diazolyl group, which 1-thia-2,3-diazolyl group is unsubstituted or substituted by one or more linear or branched C₁-C₃ alkyl groups; a benzimidazolyl group, which benzimidazolyl group is unsubstituted or substituted by one or more linear or branched C₁-C₃ alkyl groups; a chromanyl group; a 2,2-dimethyl-1,3-dioxolane group; an imidazolyl group; an indolyl group; a tetrahydropyranyl group; a tetrazolyl group; a triazolyl group; a pyrazolyl group, a benzoyl group or a pyrimidine-2,4(1H,3H)-dione group.
R₉ is a hydrogen atom; a halogen atom; a cyano group; a C₁-C₄ haloalkyl group; a linear or branched C₁-C₃ alkyl group; a C₃-C₄ cycloalkyl group; a pyridinyl group, which pyridinyl group is unsubstituted or substituted by one or more substituents selected from a hydroxy group or a C₁-C₃ alkoxy group; a triazolyl group; a tetrazolyl group, which tetrazolyl group is unsubstituted or substituted by one or more a linear or branched C₁-C₃ alkyl groups; a pyrrolidinyl group, which pyrrolidinyl group is unsubstituted or substituted by one or more —C(O)OR′₁₃ groups; a piperazinyl group, which piperazinyl group is unsubstituted or substituted by one or more substituents selected from a linear or branched C₁-C₃ alkyl group or a —S(O)₂R′₁₃ group; a morpholinyl group; —S(O)₂R₁₃ group; a —OR₁₃ group; a —C(O)OR₁₃ group; a —NR₁₃R₁₄ group or a —C(O)—(CH₂)ₙ—NR₁₃R₁₄ group; or when Y is a group R₉, the two adjacent R₉ groups, together with the carbon atoms to which they are attached, may form a benzene ring; wherein n is 0 or 1 and each R₁₃ and R₁₄ independently represent a hydrogen atom, a linear or branched C₁-C₃ alkyl group, a —(C₁-C₃ alkyl)-(C₁-C₂ alkoxy) group, a —(C₁-C₃ alkyl)-(pirrolidinyl) group; or a —(C₁-C₃ alkyl)-(morpholinyl) group; and R′₁₃ represents a hydrogen atom or a linear or branched C₁-C₃ alkyl group.

In another particularly preferred embodiment, the compound of the invention is of formula (I′),

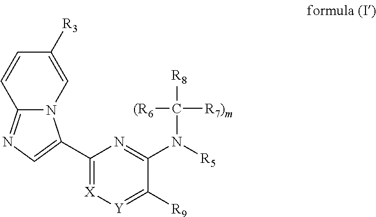

wherein X is a nitrogen atom and Y is a —CR₉ group, or Y is a nitrogen atom and X is a CR₉ group;
R₃ is a hydrogen atom, a halogen atom, a cyano group, a COOR′ group, a C₃-C₄ cycloalkyl group, a pyridine group, a pyrazole group, or a phenyl group, which phenyl group is unsubstituted or substituted with a halogen atom, wherein R′ is a hydrogen atom or a linear or branched C₁-C₄ alkyl group;
R₃ is a hydrogen atom, a halogen atom, a cyano group, a —C(O)OR′ group, a C₃-C₄ cycloalkyl group, a pyridine group, a pyrazole group, or a phenyl group, which phenyl group is unsubstituted or substituted with a halogen atom, wherein R′ is a hydrogen atom or a linear or branched C₁-C₄ alkyl group;
R₅ is a hydrogen atom, a methyl group, an ethyl group, a —CH₂-cyclopropyl group, or R₅ together with R₈ and the nitrogen atom to which R₅ is bonded form a 1,4-diazepane-1-yl group, which 1,4-diazepane-1-yl group is unsubstituted or substituted with a —C(O)CH₂CN group;
R₆ and R₇ each independently represent a hydrogen atom or a methyl group;
R₉ is a hydrogen atom, halogen atom, or a methyl, ethyl, piperazine, pyridone or pyridine group, which pyridine group is unsubstituted or substituted with a C₁-C₂ alkoxy group, or when Y is a group R₉, the two adjacent R₉ groups, together with the carbon atoms to which they are bonded, may form a benzene ring;
R₈ together with R₅ and the nitrogen atom to which R₅ is bonded form a said 1,4-diazepane-1-yl group, or R₈ is a hydrogen atom, or a linear or branched C₁-C₅ alkyl, cyclohexyl, adamantyl, pyranyl, piperidinyl, chromanyl, —(C₁-C₅ alkyl)-phenyl, —(C₁-C₅ alkyl)-cyclohexyl, —(C₁-C₅ alkyl)-(C₁-C₂ alkoxy), -A-SO₂—R′, -A-A′, -A-L-C(O)NR′R″, -A-L-CN, -A-C(O)—N(C₁-C₂ alkyl)-L-CN, -A-C(O)—NR′R″, -A-C(O)-A″, -A-C(O)—R″′, -A-CO₂—R′, -A-C(O)-L-A″′, -A-C(O)-L-CN, or -A-C(O)-L-O—R′ group, wherein R′ and R″ are the same or different and each represents a hydrogen atom or linear or a branched C₁-C₄ alkyl group, and R″′ represents a linear or branched C₁-C₅ alkyl, C₁-C₂ haloalkyl or C₁-C₄ hydroxyalkyl group, the cyclohexyl, adamantyl, pyranyl, piperidinyl and chromanyl groups being unsubstituted or substituted with a halogen atom, or a hydroxy, linear or branched C₁-C₂ alkyl, or C₁-C₂ alkoxy group, and wherein
L is a linear or branched C₁-C₅ alkylene group,
A is a piperidinyl or pyrrolidinyl group, which is unsubstituted or substituted with a C₁-C₂ alkyl group,
A′ is a phenyl or pyridinyl group, which is unsubstituted or substituted with 1 or 2 halogen atoms or CN groups,
A″ is a pyrrolidinyl, pyridinyl, pyrazolyl or cyclopropyl group, the pyrrolidinyl, pyridinyl, pyrazolyl and cyclopropyl groups being unsubstituted or substituted with 1 or 2 halogen atoms or cyano groups, and
A″′ is an imidazolyl group.

Particular individual compounds of the invention include:
3-(4-{[(1S)-1-phenylethyl]amino}pyrimidin-2-yl)imidazo[1,2-a]pyridine-6-carbonitrile;
3-(4-{[(1R)-1-phenylethyl]amino}pyrimidin-2-yl)imidazo[1,2-a]pyridine-6-carbonitrile;
3-[4-(Benzylamino)pyrimidin-2-yl]imidazo[1,2-a]pyridine-6-carbonitrile;
3-(4-{[(1S)-2-methoxy-1-methylethyl]amino}pyrimidin-2-yl)imidazo[1,2-a]pyridine-6-carbonitrile;
3-{4-[(Cyclohexylmethyl)amino]pyrimidin-2-yl}imidazo[1,2-a]pyridine-6-carbonitrile;
3-{4-[(2-Methoxyethyl)amino]pyrimidin-2-yl}imidazo[1,2-a]pyridine-6-carbonitrile;
3-{4-[(1-Adamantylmethyl)amino]pyrimidin-2-yl}imidazo[1,2-a]pyridine-6-carbonitrile;
3-{4-[(2,2-Dimethylpropyl)amino]pyrimidin-2-yl}imidazo[1,2-a]pyridine-6-carbonitrile;
3-{5-Bromo-4-[(2,2-dimethylpropyl)amino]pyrimidin-2-yl}imidazo[1,2-a]pyridine-6-carbonitrile;
3-{4-[(2,2-Dimethylpropyl)amino]-5-piperazin-1-ylpyrimidin-2-yl}imidazo[1,2-a]pyridine-6-carbonitrile;
3-[4-[(2,2-Dimethylpropyl)amino]-5-(2-methoxypyridin-4-yl)pyrimidin-2-yl]imidazo[1,2-a]pyridine-6-carbonitrile;
3-[4-[(2,2-Dimethylpropyl)amino]-5-(2-oxo-1,2-dihydropyridin-4-yl)pyrimidin-2-yl]imidazo[1,2-a]pyridine-6-carbonitrile;
3-{4-[(2,2-Dimethylpropyl)amino]-5-pyridin-3-ylpyrimidin-2-yl}imidazo[1,2-a]pyridine-6-carbonitrile;
3-{4-[(3-Fluorobenzyl)amino]pyrimidin-2-yl}imidazo[1,2-a]pyridine-6-carbonitrile;
3-{4-[(4-Fluorobenzyl)amino]pyrimidin-2-yl}imidazo[1,2-a]pyridine-6-carbonitrile;
3-{4-[(2-Methylbenzyl)amino]pyrimidin-2-yl}imidazo[1,2-a]pyridine-6-carbonitrile;
Tert-butyl-2-({[2-(6-cyanoimidazo[1,2-a]pyridin-3-yl)pyrimidin-4-yl]amino}methyl)piperidine-1-carboxylate;
3-(4-{[(1-Acetylpiperidin-2-yl)methyl]amino}pyrimidin-2-yl)imidazo[1,2-a]pyridine-6-carbonitrile;
3-[4-(Tetrahydro-2H-pyran-4-ylamino)pyrimidin-2-yl]imidazo[1,2-a]pyridine-6-carbonitrile;
3-(4-(8-Fluorochroman-4-ylamino)pyrimidin-2-yl)imidazo[1,2-a]pyridine-6-carbonitrile;
3-[4-(Cyclohexylamino)pyrimidin-2-yl]imidazo[1,2-a]pyridine-6-carbonitrile;
3-{4-[(Trans-4-hydroxycyclohexyl)amino]pyrimidin-2-yl}imidazo[1,2-a]pyridine-6-carbonitrile;
3-{4-[(5-Hydroxy-2-adamantyl)amino]pyrimidin-2-yl}imidazo[1,2-a]pyridine-6-carbonitrile;
3-{4-[(5-Hydroxy-2-adamantyl)amino]pyrimidin-2-yl}imidazo[1,2-a]pyridine-6-carbonitrile;
2-{4-[(2,2-Dimethylpropyl)amino]quinazolin-2-yl}imidazo[1,2-a]pyridine-6-carbonitrile;
3-{4-[Benzyl(methyl)amino]pyrimidin-2-yl}imidazo[1,2-a]pyridine-6-carbonitrile;
3-(4-{[(1S)-1-phenylethyl]amino}pyrimidin-2-yl)imidazo[1,2-a]pyridine-6-carboxylic acid;
2-(6-Fluoroimidazo[1,2-a]pyridin-3-yl)-N-[(1S)-1-phenylethyl]pyrimidin-4-amine;
Trans-4-{[2-(6-fluoroimidazo[1,2-a]pyridin-3-yl)pyrimidin-4-yl]amino}cyclohexanol;
3-(4-{[(2S,7S)-5-hydroxy-2-adamantyl]amino}pyrimidin-2-yl)imidazo[1,2-a]pyridine-6-carbonitrile;
N-(2,2-dimethylpropyl)-2-imidazo[1,2-a]pyridin-3-ylpyrimidin-4-amine;
N-(2,2-dimethylpropyl)-2-imidazo[1,2-a]pyridin-3-yl-6-pyridin-3-ylpyrimidin-4-amine;
3-(6-{[(1S)-1-phenylethyl]amino}pyrazin-2-yl)imidazo[1,2-a]pyridine-6-carbonitrile;
3-{6-[(Cyclohexylmethyl)amino]pyrazin-2-yl}imidazo[1,2-a]pyridine-6-carbonitrile;
6-(6-Fluoroimidazo[1,2-a]pyridin-3-yl)-N-[(1S)-1-phenylethyl]pyrazin-2-amine;
6-(6-Chloroimidazo[1,2-a]pyridin-3-yl)-N-[(1S)-1-phenylethyl]pyrazin-2-amine;
(S)-6-(imidazo[1,2-a]pyridin-3-yl)-N-(1-phenylethyl)pyrazin-2-amine;
6-(6-Cyclopropylimidazo[1,2-a]pyridin-3-yl)-N-[(1S)-1-phenylethyl]pyrazin-2-amine;
N-[(1S)-1-phenylethyl]-6-(6-pyridin-3-ylimidazo[1,2-a]pyridin-3-yl)pyrazin-2-amine;
N-[(1S)-1-phenylethyl]-6-(6-pyridin-4-ylimidazo[1,2-a]pyridin-3-yl)pyrazin-2-amine;
N-[(1S)-1-phenylethyl]-6-[6-(1H-pyrazol-4-yl)imidazo[1,2-a]pyridin-3-yl]pyrazin-2-amine;
N-[(1S)-1-phenylethyl]-6-(6-phenylimidazo[1,2-a]pyridin-3-yl)pyrazin-2-amine;
6-[6-(4-Fluorophenyl)imidazo[1,2-a]pyridin-3-yl]-N-[(1S)-1-phenylethyl]pyrazin-2-amine;
3-(4-{[(3R)-1-(ethylsulfonyl)piperidin-3-yl]amino}pyrimidin-2-yl)imidazo[1,2-a]pyridine-6-carbonitrile;
3-(4-{[(3R)-1-(isopropylsulfonyl)piperidin-3-yl]amino}pyrimidin-2-yl)imidazo[1,2-a]pyridine-6-carbonitrile;
3-(4-{[(3R)-1-(cyanoacetyl)piperidin-3-yl]amino}pyrimidin-2-yl)imidazo[1,2-a]pyridine-6-carbonitrile;
3-(4-{[(3R)-1-propionylpiperidin-3-yl]amino}pyrimidin-2-yl)imidazo[1,2-a]pyridine-6-carbonitrile;
3-[4-({(3R)-1-[(1-cyanocyclopropyl)carbonyl]piperidin-3-yl}amino)pyrimidin-2-yl]imidazo[1,2-a]pyridine-6-carbonitrile;
3-(4-{[(3R)-1-(methoxyacetyl)piperidin-3-yl]amino}pyrimidin-2-yl)imidazo[1,2-a]pyridine-6-carbonitrile;
3-(4-{[(3R)-1-(3-hydroxy-3-methylbutanoyl)piperidin-3-yl]amino}pyrimidin-2-yl)imidazo[1,2-a]pyridine-6-carbonitrile;
3-(4-{[(3R)-1-(3,3-dimethylbutanoyl)piperidin-3-yl]amino}pyrimidin-2-yl)imidazo[1,2-a]pyridine-6-carbonitrile;
3-(4-{[(3R)-1-(1H-imidazol-4-ylacetyl)piperidin-3-yl]amino}pyrimidin-2-yl)imidazo[1,2-a]pyridine-6-carbonitrile;
3-[4-({(3R)-1-[(5-cyanopyridin-2-yl)carbonyl]piperidin-3-yl}amino)pyrimidin-2-yl]imidazo[1,2-a]pyridine-6-carbonitrile;
3-(4-{[(3R)-1-(3,3,3-trifluoropropanoyl)piperidin-3-yl]amino}pyrimidin-2-yl)imidazo[1,2-a]pyridine-6-carbonitrile;
(3R)-3-{[2-(6-cyanoimidazo[1,2-a]pyridin-3-yl)pyrimidin-4-yl]amino}-N,N-dimethyl piperidine-1-carboxamide;
3-{4-[((3R)-1-{[(2S,4S)-2-cyano-4-fluoropyrrolidin-1-yl]carbonyl}piperidin-3-yl)amino]pyrimidin-2-yl}imidazo[1,2-a]pyridine-6-carbonitrile;
3-(4-{[(3R)-1-(5-cyanopyridin-2-yl)piperidin-3-yl]amino}pyrimidin-2-yl)imidazo[1,2-a]pyridine-6-carbonitrile;
3-(4-{[(3R)-1-(4-cyano-2-fluorophenyl)piperidin-3-yl]amino}pyrimidin-2-yl)imidazo[1,2-a]pyridine-6-carbonitrile;
3-{4-[[(3R)-1-(cyanoacetyl)piperidin-3-yl](methyl)amino]pyrimidin-2-yl}imidazo[1,2-a]pyridine-6-carbonitrile;
3-(4-{[(3S)-1-(cyanoacetyl)piperidin-3-yl]amino}pyrimidin-2-yl)imidazo[1,2-a]pyridine-6-carbonitrile;
Cis-3-(4-{1-(cyanoacetyl)-4-methylpiperidin-3-yl]amino}pyrimidin-2-yl)imidazo[1,2-a]pyridine-6-carbonitrile;
Cis-3-(4-{1-(ethylsulfonyl)-4-methylpiperidin-3-yl]amino}pyrimidin-2-yl)imidazo[1,2-a]pyridine-6-carbonitrile;
3-{4-[[1-(Ethylsulfonyl)-4-methylpiperidin-3-yl](methyl)amino]pyrimidin-2-yl}imidazo[1,2-a]pyridine-6-carbonitrile;
3-((3R)-3-{[2-(6-fluoroimidazo[1,2-a]pyridin-3-yl)pyrimidin-4-yl]amino}piperidin-1-yl)-3-oxopropanenitrile;
(R)-1-(3-(2-(6-fluoroimidazo[1,2-a]pyridin-3-yl)pyrimidin-4-ylamino)piperidine-1-carbonyl)cyclopropanecarbonitrile;
2-(6-Fluoroimidazo[1,2-a]pyridin-3-yl)-N-[(3R)-1-(3,3,3-trifluoropropanoyl)piperidin-3-yl]pyrimidin-4-amine;
2-(6-Fluoroimidazo[1,2-a]pyridin-3-yl)-N-[(3R)-1-(1H-pyrazol-4ylcarbonyl)piperidin-3-yl]pyrimidin-4-amine;
(3R)—N-(cyanomethyl)-3-{[2-(6-fluoroimidazo[1,2-a]pyridin-3-yl)pyrimidin-4-yl]amino}-N-methylpiperidine-1-carboxamide;
2-((3R)-3-{[2-(6-fluoroimidazo[1,2-a]pyridin-3-yl)pyrimidin-4-yl]amino}piperidin-1-yl)-2-methylpropanamide;
2-(6-Fluoroimidazo[1,2-a]pyridin-3-yl)-N-methyl-N-[(3R)-1-(3,3,3-trifluoro propanoyl)piperidin-3-yl]pyrimidin-4-amine;
(R)-3-(3-((2-(6-fluoroimidazo[1,2-a]pyridin-3-yl)pyrimidin-4-yl)(methyl)amino)piperidin-1-yl)-3-oxopropanenitrile;
(R)-1-(3-((2-(6-fluoroimidazo[1,2-a]pyridin-3-yl)pyrimidin-4-yl)(methyl)amino)piperidine-1-carbonyl)cyclopropanecarbonitrile;
3-((3R)-3-{ethyl[2-(6-fluoroimidazo[1,2-a]pyridin-3-yl)pyrimidin-4-yl]amino}piperidin-1-yl)-3-oxopropanenitrile;
N-ethyl-2-(6-fluoroimidazo[1,2-a]pyridin-3-yl)-N-[(3R)-1-(3,3,3-trifluoropropanoyl)piperidin-3-yl]pyrimidin-4-amine;
3-((3R)-3-{(cyclopropylmethyl)[2-(6-fluoroimidazo[1,2-a]pyridin-3-yl)pyrimidin-4-yl]amino}piperidin-1-yl)-3-oxopropanenitrile;
N-(cyclopropylmethyl)-2-(6-fluoroimidazo[1,2-a]pyridin-3-yl)-N-[(3R)-1-(3,3,3-trifluoropropanoyl)piperidin-3-yl]pyrimidin-4-amine;

3-((3R)-3-{[5-fluoro-2-(6-fluoroimidazo[1,2-a]pyridin-3-yl)pyrimidin-4-yl]amino}piperidin-1-yl)-3-oxopropanenitrile;
5-Fluoro-2-(6-fluoroimidazo[1,2-a]pyridin-3-yl)-N-[(3R)-1-(3,3,3-trifluoropropanoyl)piperidin-3-yl]pyrimidin-4-amine;
3-((3R)-3-{[2-(6-fluoroimidazo[1,2-a]pyridin-3-yl)-5-methylpyrimidin-4-yl]amino}piperidin-1-yl)-3-oxopropanenitrile;
2-(6-fluoroimidazo[1,2-a]pyridin-3-yl)-5-methyl-N-[(3R)-1-(3,3,3-trifluoropropanoyl)piperidin-3-yl]pyrimidin-4-amine;
3-((3R)-3-{[2-(6-fluoroimidazo[1,2-a]pyridin-3-yl)pyrimidin-4-yl]amino}pyrrolidin-1-yl)-3-oxopropanenitrile;
3-{4-[4-(cyanoacetyl)-1,4-diazepan-1-yl]pyrimidin-2-yl}imidazo[1,2-a]pyridine-6-carbonitrile;
3-((3R)-3-{[5-chloro-2-(6-fluoroimidazo[1,2-a]pyridin-3-yl)pyrimidin-4-yl]amino}piperidin-1-yl)-3-oxopropanenitrile;
3-((3R)-3-{[2-(6-fluoroimidazo[1,2-a]pyridin-3-yl)pyrimidin-4-yl]amino}piperidin-1-yl)-2,2-dimethylpropanenitrile;
2-((3R)-3-{[2-(6-fluoroimidazo[1,2-a]pyridin-3-yl)pyrimidin-4-yl]amino}piperidin-1-yl)acetamide,
(S)-2-(6-fluoroimidazo[1,2-a]pyridin-3-yl)-N-(1-(5-fluoropyridin-2-yl)ethyl)pyrimidin-4-amine;
2-(6-fluoroimidazo[1,2-a]pyridin-3-yl)-N-(pyridin-2-ylmethyl)pyrimidin-4-amine;
(S)-2-(6-fluoroimidazo[1,2-a]pyridin-3-yl)-N-(1-(4-fluorophenyl)butyl)pyrimidin-4-amine;
(R)-3-(2-(6-fluoroimidazo[1,2-a]pyridin-3-yl)pyrimidin-4-ylamino)-1-propylpyrrolidin-2-one;
6-fluoro-3-(4-(2-(pyridin-2-yl)pyrrolidin-1-yl)pyrimidin-2-yl)imidazo[1,2-a]pyridine;
(S)—N,N-diethyl-1-(2-(6-fluoroimidazo[1,2-a]pyridin-3-yl)pyrimidin-4-yl)pyrrolidine-2-carboxamide;
(R)—N,N-diethyl-1-(2-(6-fluoroimidazo[1,2-a]pyridin-3-yl)pyrimidin-4-yl)pyrrolidine-2-carboxamide;
2-((1r,4r)-4-(2-(6-fluoroimidazo[1,2-a]pyridin-3-yl)pyrimidin-4-ylamino)cyclohexyl)acetonitrile;
(R)-2-(3-(2-(6-fluoroimidazo[1,2-a]pyridin-3-yl)pyrimidin-4-ylamino)piperidin-1-ylsulfonyl)acetonitrile;
(R)-4,4,4-trifluoro-1-(3-(2-(6-fluoroimidazo[1,2-a]pyridin-3-yl)pyrimidin-4-ylamino)piperidin-1-yl)butan-1-one;
((R)-3-(2-(6-fluoroimidazo[1,2-a]pyridin-3-yl)pyrimidin-4-ylamino)piperidin-1-yl)((1R,2R)-2-phenylcyclopropyl)methanone;
(R)-1-((R)-3-(2-(6-fluoroimidazo[1,2-a]pyridin-3-yl)pyrimidin-4-ylamino)piperidin-1-yl)-2-hydroxy-3,3-dimethylbutan-1-one;
(R)-2-cyclopentyl-1-(3-(2-(6-fluoroimidazo[1,2-a]pyridin-3-yl)pyrimidin-4-ylamino)piperidin-1-yl)ethanone;
(R)-1-(2-(3-(2-(6-fluoroimidazo[1,2-a]pyridin-3-yl)pyrimidin-4-ylamino)piperidin-1-yl)-2-oxoethyl)-5-methylpyrimidine-2,4(1H,3H)-dione;
(R)-1-(3-(2-(6-fluoroimidazo[1,2-a]pyridin-3-yl)pyrimidin-4-ylamino)piperidin-1-yl)-2-(1H-1,2,4-triazol-1-yl)ethanone;
(R)-(3-(2-(6-fluoroimidazo[1,2-a]pyridin-3-yl)pyrimidin-4-ylamino)piperidin-1-yl)(1-methylcyclohexyl)methanone;
(2,2-difluorocyclopropyl)((R)-3-(2-(6-fluoroimidazo[1,2-a]pyridin-3-yl)pyrimidin-4-ylamino)piperidin-1-yl)methanone;
((R)-3-(2-(6-fluoroimidazo[1,2-a]pyridin-3-yl)pyrimidin-4-ylamino)piperidin-1-yl)((1R,2S)-2-hydroxycyclopentyl)methanone;
(R)-1-(3-(2-(6-fluoroimidazo[1,2-a]pyridin-3-yl)pyrimidin-4-ylamino)piperidin-1-yl)-2-(1H-pyrazol-1-yl)ethanone;
(R)-cyclohexyl(3-(2-(6-fluoroimidazo[1,2-a]pyridin-3-yl)pyrimidin-4-ylamino)piperidin-1-yl)methanone;
(R)-(3-(2-(6-fluoroimidazo[1,2-a]pyridin-3-yl)pyrimidin-4-ylamino)piperidin-1-yl)(4-methyl-1,2,3-thiadiazol-5-yl)methanone;
(R)-1-(3-(2-(6-fluoroimidazo[1,2-a]pyridin-3-yl)pyrimidin-4-ylamino)piperidin-1-yl)-2-(4-(hydroxymethyl)phenyl)ethanone;
(S)-1-((R)-3-(2-(6-fluoroimidazo[1,2-a]pyridin-3-yl)pyrimidin-4-ylamino)piperidin-1-yl)-3-phenylbutan-1-one;
(R)-1-((R)-3-(2-(6-fluoroimidazo[1,2-a]pyridin-3-yl)pyrimidin-4-ylamino)piperidin-1-yl)-3-phenylbutan-1-one;
(R)-1-(3-(2-(6-fluoroimidazo[1,2-a]pyridin-3-yl)pyrimidin-4-ylamino)piperidin-1-yl)-3-(4-methoxyphenyl)propan-1-one;
(R)-(5-fluoro-2,6-dihydroxypyrimidin-4-yl)(3-(2-(6-fluoroimidazo[1,2-a]pyridin-3-yl)pyrimidin-4-ylamino)piperidin-1-yl)methanone;
(R)-2-(3-chlorophenyl)-14(R)-3-(2-(6-fluoroimidazo[1,2-a]pyridin-3-yl)pyrimidin-4-ylamino)piperidin-1-yl)-2-hydroxyethanone;
(R)-(3-(2-(6-fluoroimidazo[1,2-a]pyridin-3-yl)pyrimidin-4-ylamino)piperidin-1-yl)(pyrimidin-5-yl)methanone;
(R)-(3-(2-(6-fluoroimidazo[1,2-a]pyridin-3-yl)pyrimidin-4-ylamino)piperidin-1-yl)(1-(trifluoromethyl)cyclobutyl)methanone;
(R)-(3-(2-(6-fluoroimidazo[1,2-a]pyridin-3-yl)pyrimidin-4-ylamino)piperidin-1-yl)(1-hydroxycyclopropyl)methanone;
(R)-2-(benzyloxy)-1-(3-(2-(6-fluoroimidazo[1,2-a]pyridin-3-yl)pyrimidin-4-ylamino)piperidin-1-yl)ethanone;
(R)-(3-(2-(6-fluoroimidazo[1,2-a]pyridin-3-yl)pyrimidin-4-ylamino)piperidin-1-yl)(1H-indol-2-yl)methanone;
(R)-1-(3-(2-(6-fluoroimidazo[1,2-a]pyridin-3-yl)pyrimidin-4-ylamino)piperidin-1-yl)-5-(4-fluorophenyl)pentan-1-one;
(R)-2-(2-chlorophenyl)-1-((R)-3-(2-(6-fluoroimidazo[1,2-a]pyridin-3-yl)pyrimidin-4-ylamino)piperidin-1-yl)-2-hydroxyethanone;
(R)-1-(3-(2-(6-fluoroimidazo[1,2-a]pyridin-3-yl)pyrimidin-4-ylamino)piperidin-1-yl)-2-(methylamino)ethanone;
(R)-1-(3-(2-(6-fluoroimidazo[1,2-a]pyridin-3-yl)pyrimidin-4-ylamino)piperidin-1-yl)-2-(2-methoxyethoxy)ethanone;
(R)-1-(3-(2-(6-fluoroimidazo[1,2-a]pyridin-3-yl)pyrimidin-4-ylamino)piperidin-1-yl)-2-(2-methylthiazol-4-yl)ethanone;
(R)-(3-(2-(6-fluoroimidazo[1,2-a]pyridin-3-yl)pyrimidin-4-ylamino)piperidin-1-yl)(tetrahydro-2H-pyran-4-yl)methanone;
(R)-1-(3-(2-(6-fluoroimidazo[1,2-a]pyridin-3-yl)pyrimidin-4-ylamino)piperidin-1-yl)-2-(2-methyl-1H-benzo[d]imidazol-1-yl)ethanone;
chroman-3-yl((R)-3-(2-(6-fluoroimidazo[1,2-a]pyridin-3-yl)pyrimidin-4-ylamino)piperidin-1-yl)methanone;
(R)—N-(2-(3-(2-(6-fluoroimidazo[1,2-a]pyridin-3-yl)pyrimidin-4-ylamino)piperidin-1-yl)-2-oxoethyl)-N-methylbenzamide;
(R)-2-(3-chlorophenoxy)-1-(3-(2-(6-fluoroimidazo[1,2-a]pyridin-3-yl)pyrimidin-4-ylamino)piperidin-1-yl)ethanone;
(R)-1-(3-(2-(6-fluoroimidazo[1,2-a]pyridin-3-yl)pyrimidin-4-ylamino)piperidin-1-yl)-2,2-bis(hydroxymethyl)butan-1-one;
(R)-1-(3-(2-(6-fluoroimidazo[1,2-a]pyridin-3-yl)pyrimidin-4-ylamino)piperidin-1-yl)-2-(2H-tetrazol-5-yl)ethanone;
N-(1-(1H-1,2,4-triazol-3-yl)piperidin-3-yl)-2-(6-fluoroimidazo[1,2-a]pyridin-3-yl)pyrimidin-4-amine;
(R)-2-(3-(2-(6-fluoroimidazo[1,2-a]pyridin-3-yl)pyrimidin-4-ylamino)piperidin-1-yl)thiazole-5-carbonitrile;
(R)-1-((R)-3-(2-(6-fluoroimidazo[1,2-a]pyridin-3-yl)pyrimidin-4-ylamino)piperidin-1-yl)-2,3-dihydroxypropan-1-one;
(S)-1-((R)-3-(2-(6-fluoroimidazo[1,2-a]pyridin-3-yl)pyrimidin-4-ylamino)piperidin-1-yl)-2,3-dihydroxypropan-1-one;
(R)-3-(3-(2-(6-fluoroimidazo[1,2-a]pyridin-3-yl)-5-methylpyrimidin-4-ylamino)pyrrolidin-1-yl)-3-oxopropanenitrile;
(S)-2-(6-fluoroimidazo[1,2-a]pyridin-3-yl)-N-(1-(5-fluoropyridin-2-yl)ethyl)-5-methylpyrimidin-4-amine;
2-(6-fluoroimidazo[1,2-a]pyridin-3-yl)-N44(5-fluoropyridin-2-yl)methyl)pyrimidine-4,5-diamine;
2-(6-fluoroimidazo[1,2-a]pyridin-3-yl)-N4-(5,6,7,8-tetrahydroquinolin-5-yl)pyrimidine-4,5-diamine;
(R)-2-(6-fluoroimidazo[1,2-a]pyridin-3-yl)-N4-(3-methylbutan-2-yl)pyrimidine-4,5-diamine;
2-(6-fluoroimidazo[1,2-a]pyridin-3-yl)-N4-(1-methoxypropan-2-yl)pyrimidine-4,5-diamine;
2-((1r,4r)-4-(5-amino-2-(6-fluoroimidazo[1,2-a]pyridin-3-yl)pyrimidin-4-ylamino)cyclohexyl)acetonitrile;
(1r,4r)-4-(5-amino-2-(6-fluoroimidazo[1,2-a]pyridin-3-yl)pyrimidin-4-ylamino)-1-methylcyclohexanol;
2-(6-fluoroimidazo[1,2-a]pyridin-3-yl)-N4-(1-(5-fluoropyridin-2-yl)-2-methoxyethyl)pyrimidine-4,5-diamine;
(S)-2-(6-fluoroimidazo[1,2-a]pyridin-3-yl)-4-(1-(5-fluoropyridin-2-yl)ethylamino)pyrimidine-5-carboxylic acid;
(S)-2-(6-fluoroimidazo[1,2-a]pyridin-3-yl)-4-(1-(5-fluoropyridin-2-yl)ethylamino)pyrimidine-5-carboxamide;
(S)-2-(6-fluoroimidazo[1,2-a]pyridin-3-yl)-4-((1-(5-fluoropyridin-2-yl)ethyl)(methyl)amino)pyrimidine-5-carboxylic acid;
2-(6-fluoroimidazo[1,2-a]pyridin-3-yl)-4-(pyridin-3-ylmethylamino)pyrimidine-5-carboxylic acid;
2-(6-fluoroimidazo[1,2-a]pyridin-3-yl)-4-(pyridin-3-ylmethylamino)pyrimidine-5-carboxamide;
2-(6-fluoroimidazo[1,2-a]pyridin-3-yl)-4-((5-fluoropyridin-2-yl)methylamino)pyrimidine-5-carboxylic acid;
2-(6-fluoroimidazo[1,2-a]pyridin-3-yl)-4-((5-fluoropyridin-2-yl)methylamino)pyrimidine-5-carboxamide;
(S)-2-(6-fluoroimidazo[1,2-a]pyridin-3-yl)-4-(1-(4-fluorophenyl)butylamino)pyrimidine-5-carboxylic acid;
(S)-2-(6-fluoroimidazo[1,2-a]pyridin-3-yl)-4-(1-(4-fluorophenyl)butylamino)pyrimidine-5-carboxamide;
4-((1r,4r)-4-(cyanomethyl)cyclohexylamino)-2-(6-fluoroimidazo[1,2-a]pyridin-3-yl)pyrimidine-5-carboxylic acid;
4-((1r,4r)-4-(cyanomethyl)cyclohexylamino)-2-(6-fluoroimidazo[1,2-a]pyridin-3-yl)pyrimidine-5-carboxamide;
((R)-2,2-dimethyl-1,3-dioxolan-4-yl)((R)-3-(2-(6-fluoroimidazo[1,2-a]pyridin-3-yl)-5-methylpyrimidin-4-ylamino)piperidin-1-yl)methanone;
(R)-1-((R)-3-(2-(6-fluoroimidazo[1,2-a]pyridin-3-yl)-5-methylpyrimidin-4-ylamino)piperidin-1-yl)-2,3-dihydroxypropan-1-one;
(R)-3-(3-(5-amino-2-(6-fluoroimidazo[1,2-a]pyridin-3-yl)pyrimidin-4-ylamino)piperidin-1-yl)-3-oxopropanenitrile;
(R)-3-(3-(5-cyclopropyl-2-(6-fluoroimidazo[1,2-a]pyridin-3-yl)pyrimidin-4-ylamino)piperidin-1-yl)-3-oxopropanenitrile;
(R)-4-(1-(2-cyanoacetyl)piperidin-3-ylamino)-2-(6-fluoroimidazo[1,2-a]pyridin-3-yl)pyrimidine-5-carbonitrile;
(R)-3-(3-(2-(6-fluoroimidazo[1,2-a]pyridin-3-yl)-5-(methylsulfonyl)pyrimidin-4-ylamino)piperidin-1-yl)-3-oxopropanenitrile;
(R)-ethyl 4-(1-(2-cyanoacetyl)piperidin-3-ylamino)-2-(6-fluoroimidazo[1,2-a]pyridin-3-yl)pyrimidine-5-carboxylate;
(R)-4-(1-(2-cyanoacetyl)piperidin-3-ylamino)-2-(6-fluoroimidazo[1,2-a]pyridin-3-yl)pyrimidine-5-carboxylic acid;
(R)-4-(1-(2-cyanoacetyl)piperidin-3-ylamino)-2-(6-fluoroimidazo[1,2-a]pyridin-3-yl)pyrimidine-5-carboxamide;
(R)-3-(3-(2-(6-fluoroimidazo[1,2-a]pyridin-3-yl)-5-methoxypyrimidin-4-ylamino)piperidin-1-yl)-3-oxopropanenitrile;
(R)-3-(3-(2-(6-fluoroimidazo[1,2-a]pyridin-3-yl)-6-(1H-1,2,4-triazol-1-yl)pyrimidin-4-ylamino)piperidin-1-yl)-3-oxopropanenitrile;
(R)-3-(3-(2-(6-fluoroimidazo[1,2-a]pyridin-3-yl)-6-(4-methylpiperazin-1-yl)pyrimidin-4-ylamino)piperidin-1-yl)-3-oxopropanenitrile;
(R)-3-(3-(2-(6-fluoroimidazo[1,2-a]pyridin-3-yl)-6-(piperazin-1-yl)pyrimidin-4-ylamino)piperidin-1-yl)-3-oxopropanenitrile;
(R)-3-(3-(2-(6-fluoroimidazo[1,2-a]pyridin-3-yl)-6-(4-(methylsulfonyl)piperazin-1-yl)pyrimidin-4-ylamino)piperidin-1-yl)-3-oxopropanenitrile;
(R)-3-(3-(2-(6-fluoroimidazo[1,2-a]pyridin-3-yl)-6-morpholinopyrimidin-4-ylamino)piperidin-1-yl)-3-oxopropanenitrile;
(R)-3-(3-(6-(dimethylamino)-2-(6-fluoroimidazo[1,2-a]pyridin-3-yl)pyrimidin-4-ylamino)piperidin-1-yl)-3-oxopropanenitrile;
(R)-3-(3-(2-(6-fluoroimidazo[1,2-a]pyridin-3-yl)-6-(2-morpholinoethylamino)pyrimidin-4-ylamino)piperidin-1-yl)-3-oxopropanenitrile;
(R)-3-(3-(2-(6-fluoroimidazo[1,2-a]pyridin-3-yl)-6-(2-methoxyethylamino)pyrimidin-4-ylamino)piperidin-1-yl)-3-oxopropanenitrile;
(S)-1-(6-((R)-1-(2-cyanoacetyl)piperidin-3-ylamino)-2-(6-fluoroimidazo[1,2-a]pyridin-3-yl)pyrimidin-4-yl)pyrrolidine-2-carboxylic acid;
(R)-3-(3-(2-(6-fluoroimidazo[1,2-a]pyridin-3-yl)-6-methoxypyrimidin-4-ylamino)piperidin-1-yl)-3-oxopropanenitrile;
(R)-3-(3-(2-(6-fluoroimidazo[1,2-a]pyridin-3-yl)-6-(2-(pyrrolidin-1-yl)ethoxy)pyrimidin-4-ylamino)piperidin-1-yl)-3-oxopropanenitrile;
(R)-3-(3-(2-(6-fluoroimidazo[1,2-a]pyridin-3-yl)-6-(2-morpholinoethoxy)pyrimidin-4-ylamino)piperidin-1-yl)-3-oxopropanenitrile;
(R)-3-(3-(2-(6-fluoroimidazo[1,2-a]pyridin-3-yl)-6-(2-methoxyethoxy)pyrimidin-4-ylamino)piperidin-1-yl)-3-oxopropanenitrile;
(R)-6-(1-(2-cyanoacetyl)piperidin-3-ylamino)-2-(6-fluoroimidazo[1,2-a]pyridin-3-yl)pyrimidine-4-carbonitrile;
(R)-3-(3-(2-(6-fluoroimidazo[1,2-a]pyridin-3-yl)-6-(2H-tetrazol-5-yl)pyrimidin-4-ylamino)piperidin-1-yl)-3-oxopropanenitrile;
(R)-3-(3-(2-(6-fluoroimidazo[1,2-a]pyridin-3-yl)-6-(1-methyl-1H-tetrazol-5-yl)pyrimidin-4-ylamino)piperidin-1-yl)-3-oxopropanenitrile;
(R)-3-(3-(2-(6-fluoroimidazo[1,2-a]pyridin-3-yl)-6-(2-methyl-2H-tetrazol-5-yl)pyrimidin-4-ylamino)piperidin-1-yl)-3-oxopropanenitrile;
(R)-6-(1-(2-cyanoacetyl)piperidin-3-ylamino)-2-(6-fluoroimidazo[1,2-a]pyridin-3-yl)pyrimidine-4-carboxylic acid;
(R)-6-(1-(2-cyanoacetyl)piperidin-3-ylamino)-2-(6-fluoroimidazo[1,2-a]pyridin-3-yl)pyrimidine-4-carboxamide;
(R)-3-(3-(6-amino-2-(6-fluoroimidazo[1,2-a]pyridin-3-yl)pyrimidin-4-ylamino)piperidin-1-yl)-3-oxopropanenitrile;
(R)-3-(3-(5-fluoro-2-(6-fluoroimidazo[1,2-a]pyridin-3-yl)-6-morpholinopyrimidin-4-ylamino)piperidin-1-yl)-3-oxopropanenitrile;
(R)-1-((R)-3-(5-fluoro-2-(6-fluoroimidazo[1,2-a]pyridin-3-yl)6-morpholinopyrimidin-4-ylamino)piperidin-1-yl)-2,3-dihydroxypropan-1-one;
(R)-3-(3-(2-(6-fluoroimidazo[1,2-a]pyridin-3-yl)-6-methoxy-5-(trifluoromethyl)pyrimidin-4-ylamino)piperidin-1-yl)-3-oxopropanenitrile;
   or a pharmaceutically acceptable salt, or solvate or N-oxide or stereoisomer thereof.

Of outstanding interest are:
3-(4-{[(1S)-1-phenylethyl]amino}pyrimidin-2-yl)imidazo[1,2-a]pyridine-6-carbonitrile;
3-{4-[(4-Fluorobenzyl)amino]pyrimidin-2-yl}imidazo[1,2-a]pyridine-6-carbonitrile;
2-{4-[(2,2-Dimethylpropyl)amino]quinazolin-2-yl}imidazo[1,2-a]pyridine-6-carbonitrile;
2-(6-Fluoroimidazo[1,2-a]pyridin-3-yl)-N-[(1S)-1-phenylethyl]pyrimidin-4-amine;
N-(2,2-dimethylpropyl)-2-imidazo[1,2-a]pyridin-3-yl-6-pyridin-3-ylpyrimidin-4-amine;
3-((3R)-3-{[2-(6-fluoroimidazo[1,2-a]pyridin-3-yl)pyrimidin-4-yl]amino}piperidin-1-yl)-3-oxopropanenitrile;
2-(6-Fluoroimidazo[1,2-a]pyridin-3-yl)-N-[(3R)-1-(3,3,3-trifluoropropanoyl)piperidin-3-yl]pyrimidin-4-amine;
(R)-3-(3-((2-(6-fluoroimidazo[1,2-a]pyridin-3-yl)pyrimidin-4-yl)(methyl)amino) piperidin-1-yl)-3-oxopropanenitrile;
3-((3R)-3-{[5-fluoro-2-(6-fluoroimidazo[1,2-a]pyridin-3-yl)pyrimidin-4-yl]amino}piperidin-1-yl)-3-oxopropanenitrile;
5-Fluoro-2-(6-fluoroimidazo[1,2-a]pyridin-3-yl)-N-[(3R)-1-(3,3,3-trifluoropropanoyl)piperidin-3-yl]pyrimidin-4-amine;
3-((3R)-3-{[2-(6-fluoroimidazo[1,2-a]pyridin-3-yl)-5-methylpyrimidin-4-yl]amino}piperidin-1-yl)-3-oxopropanenitrile;
2-(6-fluoroimidazo[1,2-a]pyridin-3-yl)-5-methyl-N-[(3R)-1-(3,3,3-trifluoropropanoyl)piperidin-3-yl]pyrimidin-4-amine;
   or a pharmaceutically acceptable salt, or solvate or N-oxides or stereoisomer thereof.

Preferred individual compounds of the invention include:
3-(4-{[(1S)-1-phenylethyl]amino}pyrimidin-2-yl)imidazo[1,2-a]pyridine-6-carbonitrile;
3-(4-{[(1R)-1-phenylethyl]amino}pyrimidin-2-yl)imidazo[1,2-a]pyridine-6-carbonitrile;
3-[4-(Benzylamino)pyrimidin-2-yl]imidazo[1,2-a]pyridine-6-carbonitrile;
3-(4-{[(1S)-2-methoxy-1-methylethyl]amino}pyrimidin-2-yl)imidazo[1,2-a]pyridine-6-carbonitrile;
3-{4-[(Cyclohexylmethyl)amino]pyrimidin-2-yl}imidazo[1,2-a]pyridine-6-carbonitrile;
3-{4-[(2-Methoxyethyl)amino]pyrimidin-2-yl}imidazo[1,2-a]pyridine-6-carbonitrile;
3-{4-[(1-Adamantylmethyl)amino]pyrimidin-2-yl}imidazo[1,2-a]pyridine-6-carbonitrile;
3-{4-[(2,2-Dimethylpropyl)amino]pyrimidin-2-yl}imidazo[1,2-a]pyridine-6-carbonitrile;
3-{5-Bromo-4-[(2,2-dimethylpropyl)amino]pyrimidin-2-yl}imidazo[1,2-a]pyridine-6-carbonitrile;
3-{4-[(2,2-Dimethylpropyl)amino]-5-piperazin-1-ylpyrimidin-2-yl}imidazo[1,2-a]pyridine-6-carbonitrile;
3-[4-[(2,2-Dimethylpropyl)amino]-5-(2-methoxypyridin-4-yl)pyrimidin-2-yl]imidazo[1,2-a]pyridine-6-carbonitrile;
3-[4-[(2,2-Dimethylpropyl)amino]-5-(2-oxo-1,2-dihydropyridin-4-yl)pyrimidin-2-yl]imidazo[1,2-a]pyridine-6-carbonitrile;
3-{4-[(2,2-Dimethylpropyl)amino]-5-pyridin-3-ylpyrimidin-2-yl}imidazo[1,2-a]pyridine-6-carbonitrile;
3-{4-[(3-Fluorobenzyl)amino]pyrimidin-2-yl}imidazo[1,2-a]pyridine-6-carbonitrile;
3-{4-[(4-Fluorobenzyl)amino]pyrimidin-2-yl}imidazo[1,2-a]pyridine-6-carbonitrile;
3-{4-[(2-Methylbenzyl)amino]pyrimidin-2-yl}imidazo[1,2-a]pyridine-6-carbonitrile;
Tert-butyl-2-({[2-(6-cyanoimidazo[1,2-a]pyridin-3-yl)pyrimidin-4-yl]amino}methyl)piperidine-1-carboxylate;
3-(4-{[(1-Acetylpiperidin-2-yl)methyl]amino}pyrimidin-2-yl)imidazo[1,2-a]pyridine-6-carbonitrile;
3-[4-(Tetrahydro-2H-pyran-4-ylamino)pyrimidin-2-yl]imidazo[1,2-a]pyridine-6-carbonitrile;
3-(4-(8-Fluorochroman-4-ylamino)pyrimidin-2-yl)imidazo[1,2-a]pyridine-6-carbonitrile;
3-[4-(Cyclohexylamino)pyrimidin-2-yl]imidazo[1,2-a]pyridine-6-carbonitrile;
3-{4-[(Trans-4-hydroxycyclohexyl)amino]pyrimidin-2-yl}imidazo[1,2-a]pyridine-6-carbonitrile;
3-{4-[(5-Hydroxy-2-adamantyl)amino]pyrimidin-2-yl}imidazo[1,2-a]pyridine-6-carbonitrile;
3-{4-[(5-Hydroxy-2-adamantyl)amino]pyrimidin-2-yl}imidazo[1,2-a]pyridine-6-carbonitrile;
2-{4-[(2,2-Dimethylpropyl)amino]quinazolin-2-yl}imidazo[1,2-a]pyridine-6-carbonitrile;
3-{4-[Benzyl(methyl)amino]pyrimidin-2-yl}imidazo[1,2-a]pyridine-6-carbonitrile;
3-(4-{[(1S)-1-phenylethyl]amino}pyrimidin-2-yl)imidazo[1,2-a]pyridine-6-carboxylic acid;
2-(6-Fluoroimidazo[1,2-a]pyridin-3-yl)-N-[(1S)-1-phenylethyl]pyrimidin-4-amine;
Trans-4-{[2-(6-fluoroimidazo[1,2-a]pyridin-3-yl)pyrimidin-4-yl]amino}cyclohexanol;
3-(4-{[(2S,7S)-5-hydroxy-2-adamantyl]amino}pyrimidin-2-yl)imidazo[1,2-a]pyridine-6-carbonitrile;
N-(2,2-dimethylpropyl)-2-imidazo[1,2-a]pyridin-3-ylpyrimidin-4-amine;
N-(2,2-dimethylpropyl)-2-imidazo[1,2-a]pyridin-3-yl-6-pyridin-3-ylpyrimidin-4-amine;
3-(6-{[(1S)-1-phenylethyl]amino}pyrazin-2-yl)imidazo[1,2-a]pyridine-6-carbonitrile;
3-{6-[(Cyclohexylmethyl)amino]pyrazin-2-yl}imidazo[1,2-a]pyridine-6-carbonitrile;
6-(6-Fluoroimidazo[1,2-a]pyridin-3-yl)-N-[(1S)-1-phenylethyl]pyrazin-2-amine;
6-(6-Chloroimidazo[1,2-a]pyridin-3-yl)-N-[(1S)-1-phenylethyl]pyrazin-2-amine;
(S)-6-(imidazo[1,2-a]pyridin-3-yl)-N-(1-phenylethyl)pyrazin-2-amine;
6-(6-Cyclopropylimidazo[1,2-a]pyridin-3-yl)-N-[(1S)-1-phenylethyl]pyrazin-2-amine;
N-[(1S)-1-phenylethyl]-6-(6-pyridin-3-ylimidazo[1,2-a]pyridin-3-yl)pyrazin-2-amine;
N-[(1S)-1-phenylethyl]-6-(6-pyridin-4-ylimidazo[1,2-a]pyridin-3-yl)pyrazin-2-amine;
N-[(1S)-1-phenylethyl]-6-[6-(1H-pyrazol-4-yl)imidazo[1,2-a]pyridin-3-yl]pyrazin-2-amine;
N-[(1S)-1-phenylethyl]-6-(6-phenylimidazo[1,2-a]pyridin-3-yl)pyrazin-2-amine;
6-[6-(4-Fluorophenyl)imidazo[1,2-a]pyridin-3-yl]-N-[(1S)-1-phenylethyl]pyrazin-2-amine;
3-(4-{[(3R)-1-(ethylsulfonyl)piperidin-3-yl]amino}pyrimidin-2-yl)imidazo[1,2-a]pyridine-6-carbonitrile;
3-(4-{[(3R)-1-(isopropylsulfonyl)piperidin-3-yl]amino}pyrimidin-2-yl)imidazo[1,2-a]pyridine-6-carbonitrile;
3-(4-{[(3R)-1-(cyanoacetyl)piperidin-3-yl]amino}pyrimidin-2-yl)imidazo[1,2-a]pyridine-6-carbonitrile;
3-(4-{[(3R)-1-propionylpiperidin-3-yl]amino}pyrimidin-2-yl)imidazo[1,2-a]pyridine-6-carbonitrile;

3-[4-({(3R)-1-[(1-cyanocyclopropyl)carbonyl]piperidin-3-yl}amino)pyrimidin-2-yl]imidazo[1,2-a]pyridine-6-carbonitrile;
3-(4-{[(3R)-1-(methoxyacetyl)piperidin-3-yl]amino}pyrimidin-2-yl)imidazo[1,2-a]pyridine-6-carbonitrile;
3-(4-{[(3R)-1-(3-hydroxy-3-methylbutanoyl)piperidin-3-yl]amino}pyrimidin-2-yl)imidazo[1,2-a]pyridine-6-carbonitrile;
3-(4-{[(3R)-1-(3,3-dimethylbutanoyl)piperidin-3-yl]amino}pyrimidin-2-yl)imidazo[1,2-a]pyridine-6-carbonitrile;
3-(4-{[(3R)-1-(1H-imidazol-4-ylacetyl)piperidin-3-yl]amino}pyrimidin-2-yl)imidazo[1,2-a]pyridine-6-carbonitrile;
3-[4-({(3R)-1-[(5-cyanopyridin-2-yl)carbonyl]piperidin-3-yl}amino)pyrimidin-2-yl]imidazo[1,2-a]pyridine-6-carbonitrile;
3-(4-{[(3R)-1-(3,3,3-trifluoropropanoyl)piperidin-3-yl]amino}pyrimidin-2-yl)imidazo[1,2-a]pyridine-6-carbonitrile;
(3R)-3-{[2-(6-cyanoimidazo[1,2-a]pyridin-3-yl)pyrimidin-4-yl]amino}-N,N-dimethyl piperidine-1-carboxamide;
3-{4-[((3R)-1-{[(2S,4S)-2-cyano-4-fluoropyrrolidin-1-yl]carbonyl}piperidin-3-yl)amino]pyrimidin-2-yl}imidazo[1,2-a]pyridine-6-carbonitrile;
3-(4-{[(3R)-1-(5-cyanopyridin-2-yl)piperidin-3-yl]amino}pyrimidin-2-yl)imidazo[1,2-a]pyridine-6-carbonitrile;
3-(4-{[(3R)-1-(4-cyano-2-fluorophenyl)piperidin-3-yl]amino}pyrimidin-2-yl)imidazo[1,2-a]pyridine-6-carbonitrile;
3-{4-[[(3R)-1-(cyanoacetyl)piperidin-3-yl](methyl)amino]pyrimidin-2-yl}imidazo[1,2-a]pyridine-6-carbonitrile;
3-(4-{[(3S)-1-(cyanoacetyl)piperidin-3-yl]amino}pyrimidin-2-yl)imidazo[1,2-a]pyridine-6-carbonitrile;
Cis-3-(4-{1-(cyanoacetyl)-4-methylpiperidin-3-yl]amino}pyrimidin-2-yl)imidazo[1,2-a]pyridine-6-carbonitrile;
Cis-3-(4-{1-(ethylsulfonyl)-4-methylpiperidin-3-yl]amino}pyrimidin-2-yl)imidazo[1,2-a]pyridine-6-carbonitrile;
3-{4-[[1-(Ethylsulfonyl)-4-methylpiperidin-3-yl](methyl)amino]pyrimidin-2-yl}imidazo[1,2-a]pyridine-6-carbonitrile;
3-((3R)-3-{[2-(6-fluoroimidazo[1,2-a]pyridin-3-yl)pyrimidin-4-yl]amino}piperidin-1-yl)-3-oxopropanenitrile;
(R)-1-(3-(2-(6-fluoroimidazo[1,2-a]pyridin-3-yl)pyrimidin-4-ylamino)piperidine-1-carbonyl)cyclopropanecarbonitrile;
2-(6-Fluoroimidazo[1,2-a]pyridin-3-yl)-N-[(3R)-1-(3,3,3-trifluoropropanoyl)piperidin-3-yl]pyrimidin-4-amine;
2-(6-Fluoroimidazo[1,2-a]pyridin-3-yl)-N-[(3R)-1-(1H-pyrazol-4ylcarbonyl)piperidin-3-yl]pyrimidin-4-amine;
(3R)—N-(cyanomethyl)-3-{[2-(6-fluoroimidazo[1,2-a]pyridin-3-yl)pyrimidin-4-yl]amino}-N-methylpiperidine-1-carboxamide;
2-((3R)-3-{[2-(6-fluoroimidazo[1,2-a]pyridin-3-yl)pyrimidin-4-yl]amino}piperidin-1-yl)-2-methylpropanamide;
2-(6-Fluoroimidazo[1,2-a]pyridin-3-yl)-N-methyl-N-[(3R)-1-(3,3,3-trifluoro propanoyl)piperidin-3-yl]pyrimidin-4-amine;
(R)-3-(3-((2-(6-fluoroimidazo[1,2-a]pyridin-3-yl)pyrimidin-4-yl)(methyl)amino)piperidin-1-yl)-3-oxopropanenitrile;
(R)-1-(3-((2-(6-fluoroimidazo[1,2-a]pyridin-3-yl)pyrimidin-4-yl)(methyl)amino)piperidine-1-carbonyl)cyclopropanecarbonitrile;
3-((3R)-3-{ethyl[2-(6-fluoroimidazo[1,2-a]pyridin-3-yl)pyrimidin-4-yl]amino}piperidin-1-yl)-3-oxopropanenitrile;
N-ethyl-2-(6-fluoroimidazo[1,2-a]pyridin-3-yl)-N-[(3R)-1-(3,3,3-trifluoropropanoyl)piperidin-3-yl]pyrimidin-4-amine;
3-((3R)-3-{(cyclopropylmethyl)[2-(6-fluoroimidazo[1,2-a]pyridin-3-yl)pyrimidin-4-yl]amino}piperidin-1-yl)-3-oxopropanenitrile;
N-(cyclopropylmethyl)-2-(6-fluoroimidazo[1,2-a]pyridin-3-yl)-N-[(3R)-1-(3,3,3-trifluoropropanoyl)piperidin-3-yl]pyrimidin-4-amine;
3-((3R)-3-{[5-fluoro-2-(6-fluoroimidazo[1,2-a]pyridin-3-yl)pyrimidin-4-yl]amino}piperidin-1-yl)-3-oxopropanenitrile;
5-Fluoro-2-(6-fluoroimidazo[1,2-a]pyridin-3-yl)-N-[(3R)-1-(3,3,3-trifluoropropanoyl)piperidin-3-yl]pyrimidin-4-amine;
3-((3R)-3-{[2-(6-fluoroimidazo[1,2-a]pyridin-3-yl)-5-methylpyrimidin-4-yl]amino}piperidin-1-yl)-3-oxopropanenitrile;
2-(6-fluoroimidazo[1,2-a]pyridin-3-yl)-5-methyl-N-[(3R)-1-(3,3,3-trifluoropropanoyl)piperidin-3-yl]pyrimidin-4-amine;
3-((3R)-3-{[2-(6-fluoroimidazo[1,2-a]pyridin-3-yl)pyrimidin-4-yl]amino}pyrrolidin-1-yl)-3-oxopropanenitrile;
3-{4-[4-(cyanoacetyl)-1,4-diazepan-1-yl]pyrimidin-2-yl}imidazo[1,2-a]pyridine-6-carbonitrile;
3-((3R)-3-{[5-chloro-2-(6-fluoroimidazo[1,2-a]pyridin-3-yl)pyrimidin-4-yl]amino}piperidin-1-yl)-3-oxopropanenitrile;
3-((3R)-3-{[2-(6-fluoroimidazo[1,2-a]pyridin-3-yl)pyrimidin-4-yl]amino}piperidin-1-yl)-2,2-dimethylpropanenitrile;
2-((3R)-3-{[2-(6-fluoroimidazo[1,2-a]pyridin-3-yl)pyrimidin-4-yl]amino}piperidin-1-yl)acetamide,
   or a pharmaceutically acceptable salt, or solvate or N-oxide or stereoisomer thereof.

The invention further provides new imidazopyridine derivatives of formula (I), or a pharmaceutically acceptable salt, or solvate, or N-oxide, or stereoisomer or deuterated derivative thereof:

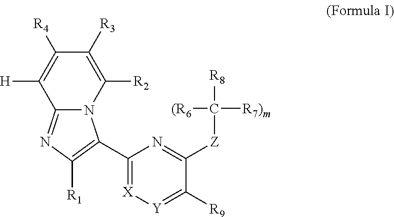

wherein m, X, Y, Z and R₁ to R₉ are as defined above, other than:
2-Pyrazinamine, 6-imidazo[1,2-a]pyridin-3-yl-N-(3R)-3-piperidinyl-, hydrochloride;
2-Pyrazinamine, 6-imidazo[1,2-a]pyridin-3-yl-N-(3S)-3-piperidinyl-;
2-Pyrazinamine, 6-(6-chloroimidazo[1,2-a]pyridin-3-yl)-N-(3R)-3-piperidinyl-;
2-Pyrazinamine, 6-(6-methoxyimidazo[1,2-a]pyridin-3-yl)-N-(3R)-3-piperidinyl-;
2-Pyrazinamine, 6-imidazo[1,2-a]pyridin-3-yl-N-(3R)-3-pyrrolidinyl-;
2-Pyrazinamine, N-(4,4-difluoro-3-piperidinyl)-6-imidazo[1,2-a]pyridin-3-yl-, hydrochloride;
2-Pyrazinamine, 6-imidazo[1,2-a]pyridin-3-yl-N-[(3R,4R)-4-methyl-3-pyrrolidinyl]-;
2-Pyrazinamine, N-(3R)-3-pyrrolidinyl-6-[6-(trifluoromethyl)imidazo[1,2-a]pyridin-3-yl]-;
Imidazo[1,2-a]pyridine-6-methanol, α,α-dimethyl-3-[6-[[(3R,4R)-4-methyl-3-pyrrolidinyl]amino]-2-pyrazinyl]-;
2-Pyrazinamine, 6-imidazo[1,2-a]pyridin-3-yl-3-methyl-N-(3R)-3-piperidinyl-;
1-Azabicyclo[2.2.2]octan-3-amine, N-[6-(7-methoxyimidazo[1,2-a]pyridin-3-yl)-2-pyrazinyl]-2-methyl-, (2R,3R)-;
3-imidazo[1,2-a]pyridin-3-yl-5-[(3R)-3-piperidinylamino]-2-pyrazinecarbonitrile, formate salt;
Imidazo[1,2-a]pyridine-7-carbonitrile, 3-[6-[(3R)-3-piperidinylamino]-2-pyrazinyl]-;
2-Pyrazinamine, N-[(3R,5S)-5-(difluoromethyl)-3-pyrrolidinyl]-6-imidazo[1,2-a]pyridin-3-yl-;
2-Pyrazinamine, N-[(3S,4R)-4-fluoro-3-piperidinyl]-6-imidazo[1,2-a]pyridin-3-yl-;
2-Pyrazinamine, N-[(3S,4S)-4-fluoro-3-piperidinyl]-6-imidazo[1,2-a]pyridin-3-yl-;
2-Pyrazinamine, 6-imidazo[1,2-a]pyridin-3-yl-N-[(3S,4S)-4-methoxy-3-piperidinyl]-;
6-imidazo[1,2-a]pyridin-3-yl-N-[(3R,6S)-6-methyl-3-piperidinyl]-2-pyrazinamine, formate salt;
6-imidazo[1,2-a]pyridin-3-yl-N-[(3R,6R)-6-methyl-3-piperidnyl]-2-pyrazinamine, formate salt;
2-Pyrazinamine, 6-[7-[2-(1-methylethoxy)ethoxy]imidazo[1,2-a]pyridin-3-yl]-N-(3R)-3-piperidinyl-;
2-Pyrazinamine, N-[(3S,4S)-4-ethoxy-3-piperidinyl]-6-imidazo[1,2-a]pyridin-3-yl-;
N-[(3S,4S)-4-ethoxy-piperidin-3-yl]-6-(imidazo[1,2a]pyridin-3-yl)pyrazin-2-amine;
2-Pyrazinamine, 6-imidazo[1,2-a]pyridin-3-yl-3-methoxy-N-(3R)-3-piperidinyl-, 2,2,2-trifluoroacetate;
2-Pyrazinamine, 6-(7-methoxyimidazo[1,2-a]pyridin-3-yl)-N-(3R)-3-piperidinyl-;
2-Pyrazinamine, 6-(7-methylimidazo[1,2-a]pyridin-3-yl)-N-(3R)-3-piperidinyl-;
2-Pyrazinamine, N-(3R)-3-piperidinyl-6-[6-(trifluoromethyl)imidazo[1,2-a]pyridin-3-yl]-;
2-Pyrazinamine, N-(3R)-3-piperidinyl-6-[7-(trifluoromethyl)imidazo[1,2-a]pyridin-3-yl]-;
2-Pyrazinamine, 6-(7-ethylimidazo[1,2-a]pyridin-3-yl)-N-(3R)-3-piperidinyl-;
2-Pyrazinamine, 6-[6-(1-methylethyl)imidazo[1,2-a]pyridin-3-yl]-N-(3R)-3-piperidinyl-;
2-Pyrazinamine, 6-(7-chloroimidazo[1,2-a]pyridin-3-yl)-N-(3R)-3-piperidinyl-;
2-Pyrazinamine, N-[(3R,4R)-4-fluoro-3-pyrrolidinyl]-6-imidazo[1,2-a]pyridin-3-yl-;
2-Pyrazinamine, 6-[5-[(3-methyl-3-oxetanyl)methoxy]-1H-benzimidazol-1-yl]-N-(3R)-3-piperidinyl-;
2-Pyrazinamine, 6-imidazo[1,2-a]pyridin-3-yl-N-(3S)-3-pyrrolidinyl-;
2-Pyrazinamine, 6-(6-methylimidazo[1,2-a]pyridin-3-yl)-N-(3R)-3-pyrrolidinyl-;
2-Pyrazinamine, 6-(7-methylimidazo[1,2-a]pyridin-3-yl)-N-(3R)-3-pyrrolidinyl-;
2-Pyrazinamine, 6-(7-methoxyimidazo[1,2-a]pyridin-3-yl)-N-(3R)-3-pyrrolidinyl-;
2-Pyrazinamine, N-(3R)-3-pyrrolidinyl-6-[7-(trifluoromethyl)imidazo[1,2-a]pyridin-3-yl]-;
2-Pyrazinamine, 6-imidazo[1,2-a]pyridin-3-yl-N-(3-methyl-3-piperidinyl)-;
2-Pyrazinamine, 6-(6-methoxyimidazo[1,2-a]pyridin-3-yl)-N-(3R)-3-pyrrolidinyl-;
2-Pyrazinamine, 6-(7-ethylimidazo[1,2-a]pyridin-3-yl)-N-(3R)-3-pyrrolidinyl-;
2-Pyrazinamine, 6-[6-(1-methylethyl)imidazo[1,2-a]pyridin-3-yl]-N-(3R)-3-pyrrolidinyl-;
2-Pyrazinamine, 6-(5-methylimidazo[1,2-a]pyridin-3-yl)-N-(3R)-3-pyrrolidinyl-;
2-Pyrazinamine, 6-(7-chloroimidazo[1,2-a]pyridin-3-yl)-N-[(3S,4S)-4-fluoro-3-pyrrolidinyl]-;
2-Pyrazinamine, N-[(3R,5S)-5-(fluoromethyl)-3-pyrrolidinyl]-6-imidazo[1,2-a]pyridin-3-yl-;
2-Pyrazinamine, N-[(3R,5S)-5-(fluoromethyl)-3-pyrrolidinyl]-6-[7-(trifluoromethyl)imidazo[1,2-a]pyridin-3-yl]-;
3-{6-[(3R)-piperidin-3-ylamino]pyrazin-2-yl}imidazo[1,2a]pyridine-7-carboxamide;
2-Pyrazinamine, 6-(6-fluoroimidazo[1,2-a]pyridin-3-yl)-N-(3R)-3-piperidinyl-;
2-Pyrazinamine, 6-(6-fluoroimidazo[1,2-a]pyridin-3-yl)-N-[(3R,6R)-6-methyl-3-piperidinyl]-;
2-Pyrazinamine, N-[(3R,5S)-5-(difluoromethyl)-3-pyrrolidinyl]-6-[7-(trifluoromethyl)imidazo[1,2-a]pyridin-3-yl];
2-Pyrazinamine, 6-(6-fluoroimidazo[1,2-a]pyridin-3-yl)-N-[(3S,4R)-4-fluoro-3-pyrrolidinyl]-;
2-Pyrazinamine, 6-(6-fluoroimidazo[1,2-a]pyridin-3-yl)-N-(3R)-3-pyrrolidinyl-;
2-Pyrazinamine, 6-(6-fluoroimidazo[1,2-a]pyridin-3-yl)-N-[(3S,4S)-4-fluoro-3-pyrrolidinyl]-;
2-Pyrazinamine, 6-(6-fluoroimidazo[1,2-a]pyridin-3-yl)-N-[(3R,6S)-6-methyl-3-piperidinyl]-;
2-Pyrazinamine, 6-(6-fluoroimidazo[1,2-a]pyridin-3-yl)-N-[(2R,3R)-2-methyl-3-piperidinyl]-;
2-Pyrazinamine, 6-(6-fluoroimidazo[1,2-a]pyridin-3-yl)-N-[(2S,3R)-2-methyl-3-piperidinyl]-;
6-imidazo[1,2-a]pyridin-3-yl-N-(3-methylpyrrolidin-3-yl)pyrazin-2-amine;
2-Pyrazinamine, 6-imidazo[1,2-a]pyridin-3-yl-N-[(2S,3R)-2-methyl-3-piperidinyl]-;
2-Pyrazinamine, 6-(7-chloroimidazo[1,2-a]pyridin-3-yl)-N-[(2R,3R)-2-methyl-3-piperidinyl]-;
2-Pyrazinamine, 6-(7-chloroimidazo[1,2-a]pyridin-3-yl)-N-[(2S,3R)-2-methyl-3-piperidinyl];
2-Pyrazinamine, 6-imidazo[1,2-a]pyridin-3-yl-N-[(2R,3R)-2-methyl-3-piperidinyl]-;
2-Pyrazinamine, N-(3R)-3-piperidinyl-6-[7-(2,2,2-trifluoro-1-methylethoxy)imidazo[1,2-a]pyridin-3-yl]-;
2-Pyrazinamine, 6-[7-[1-(methoxymethyl)propoxy]imidazo[1,2-a]pyridin-3-yl]-N-(3R)-3-piperidinyl-;
2-Azabicyclo[2.2.1]heptan-6-amine, N-(6-imidazo[1,2-a]pyridin-3-yl-2-pyrazinyl)-, (1S,4R,6R)-;
2-Azabicyclo[2.2.1]heptan-6-amine, N-[6-(7-methoxyimidazo[1,2-a]pyridin-3-yl)-2-pyrazinyl]-, (1S,4R,6R)-;
2-Azabicyclo[2.2.1]heptan-6-amine, N-[6-(6-fluoroimidazo[1,2-a]pyridin-3-yl)-2-pyrazinyl]-, (1S,4R,6R)-;
2-Pyrazinamine, 6-(7-chloroimidazo[1,2-a]pyridin-3-yl)-N-[(3R,4R)-4-methyl-3-pyrrolidinyl]-;
2-Pyrazinamine, N-[(3R,4R)-4-(fluoromethyl)-3-pyrrolidinyl]-6-imidazo[1,2-a]pyridin-3-yl-;
2-Pyrazinamine, 6-imidazo[1,2-a]pyridin-3-yl-N-[(2S,3R)-2-methyl-3-piperidinyl]-;
2-Pyrazinamine, 6-imidazo[1,2-a]pyridin-3-yl-N-[(2R,3S)-2-methyl-3-piperidinyl]-;
2-Pyrazinamine, N-(3R)-3-pyrrolidinyl-6-[7-(2,2,2-trifluoro-1-methylethoxy)imidazo[1,2-a]pyridin-3-yl]-;
Imidazo[1,2-a]pyridin-7-ol, 3-[6-[(3R)-3-piperidinylamino]-2-pyrazinyl]-;
2-Pyrazinamine, 6-imidazo[1,2-a]pyridin-3-yl-N-[(2R,3R)-2-methyl-3-piperidinyl]-;
2-Pyrazinamine, 6-imidazo[1,2-a]pyridin-3-yl-N-[(2S,3S)-2-methyl-3-piperidinyl]-;
2-Pyrazinamine, 6-(6-fluoroimidazo[1,2-a]pyridin-3-yl)-N-[(3S,4S)-4-methyl-3-pyrrolidinyl]-;
2-Pyrazinamine, 6-imidazo[1,2-a]pyridin-3-yl-N-[(3R,5R)-5-methyl-3-pyrrolidinyl]-;
2-Pyrazinamine, 6-[7-(3,3-dimethylbutoxy)imidazo[1,2-a]pyridin-3-yl]-N-(3R)-3-piperidinyl-;
Imidazo[1,2-a]pyridine-6-methanol, 3-[6-[(3R)-3-piperidinylamino]-2-pyrazinyl]-;
2-Pyrazinamine, 6-(7-methoxyimidazo[1,2-a]pyridin-3-yl)-N-[(3R,4R)-4-phenyl-3-pyrrolidinyl]-;
2-Pyrazinamine, 6-(2-methylimidazo[1,2-a]pyridin-3-yl)-N-(3R)-3-piperidinyl-;
2-Pyrazinamine, 6-[7-[(3-methyl-3-oxetanyl)methoxy]imidazo[1,2-a]pyridin-3-yl]-N-(3R)-3-pyrrolidinyl-;
2-Pyrazinamine, 6-[7-[(3-methyl-3-oxetanyl)methoxy]imidazo[1,2-a]pyridin-3-yl]-N-(3R)-3-piperidinyl-;
2-Pyrazinamine, 6-imidazo[1,2-a]pyridin-3-yl-N-[(3R,5R)-5-methoxy-3-piperidinyl]-;
2-Pyrazinamine, 6-imidazo[1,2-a]pyridin-3-yl-N-[(3R,5S)-5-(methoxymethyl)-3-pyrrolidinyl]-;
2-Pyrazinamine, 6-imidazo[1,2-a]pyridin-3-yl-N-[(3R,5S)-5-[(1-methylethoxy)methyl]-3-pyrrolidinyl]-;
2-Azabicyclo[2.2.1]heptan-6-amine, N-(6-imidazo[1,2-a]pyridin-3-yl-2-pyrazinyl)-;
Imidazo[1,2-a]pyridine-7-carbonitrile, 3-[6-[(1S,4R,6R)-2-azabicyclo[2.2.1]hept-6-ylamino]-2-pyrazinyl]-;
2-Pyrazinamine, 6-(6-fluoroimidazo[1,2-a]pyridin-3-yl)-N-[(2R,3S)-2-methyl-3-piperidinyl]-;
2-Pyrazinamine, 6-(6-fluoroimidazo[1,2-a]pyridin-3-yl)-N-[(2R,3R)-2-methyl-3-piperidinyl]-;
2-Pyrazinamine, 6-(6-fluoroimidazo[1,2-a]pyridin-3-yl)-N-[(2S,3R)-2-methyl-3-piperidinyl]-;
Imidazo[1,2-a]pyridine-7-carbonitrile, 3-[6-[(3R)-3-pyrrolidinylamino]-2-pyrazinyl]-;
Imidazo[1,2-a]pyridine-7-carbonitrile, 3-[6-[[(3R,4R)-4-methyl-3-pyrrolidinyl]amino]-2-pyrazinyl]-;
Imidazo[1,2-a]pyridine-7-carbonitrile, 3-[6-[[(3R,5S)-5-(methoxymethyl)-3-pyrrolidinyl]amino]-2-pyrazinyl]-;
Imidazo[1,2-a]pyridine-7-carbonitrile, 3-[6-[[(3R,5R)-5-methyl-3-pyrrolidinyl]amino]-2-pyrazinyl]-;
2-Pyrazinamine, 6-imidazo[1,2-a]pyridin-3-yl-N-[(3S,4R)-4-methoxy-3-pyrrolidinyl]-;
Imidazo[1,2-a]pyridine-7-carboxamide, 3-[6-[[(3R,4R)-4-methyl-3-pyrrolidinyl]amino]-2-pyrazinyl]-;
2-Pyrazinamine, 6-imidazo[1,2-a]pyridin-3-yl-N-[(3R,5S)-5-(propoxymethyl)-3-pyrrolidinyl]-;
2-Pyrazinamine, N-[(3R,5S)-5-(ethoxymethyl)-3-pyrrolidinyl]-6-imidazo[1,2-a]pyridin-3-yl-;
2-Pyrazinamine, 6-imidazo[1,2-a]pyridin-3-yl-N-[(3R,4R)-4-(1-methylethyl)-3-pyrrolidinyl]-;
2-Pyrazinamine, 6-imidazo[1,2-a]pyridin-3-yl-N-[(3S,4R)-4-(2,2,2-trifluoroethoxy)-3-pyrrolidinyl]-;
2-Pyrazinamine, N-[(3R,4R)-4-ethyl-3-pyrrolidinyl]-6-imidazo[1,2-a]pyridin-3-yl-;
2-Pyrazinamine, N-[(3R,4R)-4-cyclopropyl-3-pyrrolidinyl]-6-imidazo[1,2-a]pyridin-3-yl-;
3-Piperidinol, 5-[(6-imidazo[1,2-a]pyridin-3-yl-2-pyrazinyl)amino]-, (3R,5R)-;
2-Pyrazinamine, 6-imidazo[1,2-a]pyridin-3-yl-N-[(3R,4R)-4-methyl-3-piperidinyl]-;
2-Pyrazinamine, 6-(7-chloroimidazo[1,2-a]pyridin-3-yl)-N-[(3R,4R)-4-methyl-3-piperidinyl]-;
2-Pyrazinamine, 6-(8-methylimidazo[1,2-a]pyridin-3-yl)-N-(3R)-3-piperidinyl-;
2-Pyrazinamine, 6-[7-(1-methyl-1H-pyrazol-4-yl)imidazo[1,2-a]pyridin-3-yl]-N-[(3R,4R)-4-methyl-3-pyrrolidinyl]-;
2-Pyrazinamine, N-[(3R,4R)-4-cyclopentyl-3-pyrrolidinyl]-6-(7-methoxyimidazo[1,2-a]pyridin-3-yl)-;
2-Pyrazinamine, N-[(3R,4R)-4-cyclopropyl-3-pyrrolidinyl]-6-(7-methoxyimidazo[1,2-a]pyridin-3-yl)-;
2-Pyrazinamine, 6-imidazo[1,2-a]pyridin-3-yl-3-methoxy-N-(3R)-3-pyrrolidinyl-;
2-Pyrazinamine, 6-[7-[2-(1-methylethoxy)ethoxy]imidazo[1,2-a]pyridin-3-yl]-N-(3R)-3-pyrrolidinyl-;
2-Pyrazinamine, 6-imidazo[1,2-a]pyridin-3-yl-3-methyl-N-(3R)-3-pyrrolidinyl-;
2-Pyrazinamine, 6-(6-chloroimidazo[1,2-a]pyridin-3-yl)-N-[(2S,3R)-2-methyl-3-piperidinyl]-;
2-Pyrazinamine, 6-(6-chloroimidazo[1,2-a]pyridin-3-yl)-N-[(2R,3R)-2-methyl-3-piperidinyl]-;
2-Pyrazinamine, N-[(2S,3R)-2-methyl-3-piperidinyl]-6-[6-(trifluoromethyl)imidazo[1,2-a]pyridin-3-yl]-;
2-Pyrazinamine, N-[(2R,3R)-2-methyl-3-piperidinyl]-6-[6-(trifluoromethyl)imidazo[1,2-a]pyridin-3-yl]-;
2-Pyrazinamine, 6-(6-chloroimidazo[1,2-a]pyridin-3-yl)-N-[(3R,4R)-4-methyl-3-pyrrolidinyl]-;
2-Pyrazinamine, 6-(6-chloroimidazo[1,2-a]pyridin-3-yl)-N-(4,4-difluoro-3-piperidinyl)-;
2-Pyrazinamine, N-(4,4-difluoro-3-piperidinyl)-6-[6-(trifluoromethyl)imidazo[1,2-a]pyridin-3-yl]-;
Imidazo[1,2-a]pyridine-6-methanol, α,α-dimethyl-3-[6-[(3R)-3-piperidinylamino]-2-pyrazinyl]-;
Imidazo[1,2-a]pyridin-6-amine, 3-[6-[(3R)-3-piperidinylamino]-2-pyrazinyl]-;
2-Pyrazinamine, 6-(7-fluoroimidazo[1,2-a]pyridin-3-yl)-N-(3R)-3-piperidinyl-;
2-Pyrazinamine, 6-(7-fluoroimidazo[1,2-a]pyridin-3-yl)-N-[(3R,5S)-5-(methoxymethyl)-3-pyrrolidinyl]-;
2-Pyrrolidinemethanol, 4-[(6-imidazo[1,2-a]pyridin-3-yl-2-pyrazinyl)amino]-, (2S,4R)-;
2-Pyrazinamine, N-[(3R,5S)-5-(methoxymethyl)-3-pyrrolidinyl]-6-(7-methylimidazo[1,2-a]pyridin-3-yl)-;
2-Pyrazinamine, N-[(3R,5S)-5-[(1-methylethoxy)methyl]-3-pyrrolidinyl]-6-(7-methylimidazo[1,2-a]pyridin-3-yl)-;
2-Pyrazinamine, N-[(3R,5S)-5-(ethoxymethyl)-3-pyrrolidinyl]-6-(7-methylimidazo[1,2-a]pyridin-3-yl)-;
2-Pyrazinamine, 6-(7-methoxyimidazo[1,2-a]pyridin-3-yl)-N-[(3R,5S)-5-[(1-methylethoxy)methyl]-3-pyrrolidinyl]-;
2-Pyrazinamine, N-[(3R,5S)-5-(ethoxymethyl)-3-pyrrolidinyl]-6-(7-methoxyimidazo[1,2-a]pyridin-3-yl)-;
2-Pyrazinamine, 6-(7-chloroimidazo[1,2-a]pyridin-3-yl)-N-[(3R,5S)-5-(methoxymethyl)-3-pyrrolidinyl]-;
2-Pyrrolidinemethanol, 4-[[6-(7-chloroimidazo[1,2-a]pyridin-3-yl)-2-pyrazinyl]amino]-, (2S,4R)-;
2-Pyrazinamine, 6-(6-chloroimidazo[1,2-a]pyridin-3-yl)-N-[(3R,5S)-5-(methoxymethyl)-3-pyrrolidinyl]-;
2-Pyrazinamine, 6-imidazo[1,2-a]pyridin-3-yl-5-methyl-N-(3R)-3-piperidinyl-;
2-Pyrazinamine, N-[(3R)-4,4-difluoro-3-piperidinyl]-6-imidazo[1,2-a]pyridin-3-yl-;
2-Pyrazinamine, N-[(3S)-4,4-difluoro-3-piperidinyl]-6-imidazo[1,2-a]pyridin-3-yl-;
2-Pyrazinamine, 6-imidazo[1,2-a]pyridin-3-yl-N-[(3S,5S)-5-methoxy-3-piperidinyl]-;
2-Pyrazinamine, 6-imidazo[1,2-a]pyridin-3-yl-N-[(3S,4S)-4-methoxy-3-piperidinyl]-;
2-Pyrazinamine, 6-imidazo[1,2-a]pyridin-3-yl-N-[(3R,4R)-4-methoxy-3-piperidinyl]-, hydrochloride;
Imidazo[1,2-a]pyridine-7-carbonitrile, 3-[6-[[(2R,3R)-2-methyl-3-piperidinyl]amino]-2-pyrazinyl]-;
Imidazo[1,2-a]pyridine-6-carbonitrile, 3-[6-[[(3R,4R)-4-cyclopropyl-3-pyrrolidinyl]amino]-2-pyrazinyl];
Imidazo[1,2-a]pyridine-7-carbonitrile, 3-[6-[[(3R,4R)-4-cyclopropyl-3-pyrrolidinyl]amino]-2-pyrazinyl]-;
1-Azabicyclo[2.2.2]octan-3-amine, N-[6-(7-methoxyimidazo[1,2-a]pyridin-3-yl)-2-pyrazinyl]-2-methyl-, (2S,3S)-;
2-Pyrazinamine, 6-(6-fluoroimidazo[1,2-a]pyridin-3-yl)-N-[(3R,5S)-5-(methoxymethyl)-3-pyrrolidinyl]-;
2-Pyrazinecarbonitrile, 3-imidazo[1,2-a]pyridin-3-yl-5-[(3R)-3-pyrrolidinylamino]-;
2-Pyrazinamine, N-(3S)-3-pyrrolidinyl-6-[6-(trifluoromethyl)imidazo[1,2-a]pyridin-3-yl]-;
Ethanol, 2-[[3-[6-[(3R)-3-pyrrolidinylamino]-2-pyrazinyl]imidazo[1,2-a]pyridin-7-yl]oxy]-1-Propanol, 2-[[3-[6-[(3R)-3-pyrrolidinylamino]-2-pyrazinyl]imidazo[1,2-a]pyridin-7-yl]oxy]-, (2S);
1-Propanol, 2-[[3-[6-[(3R)-3-piperidinylamino]-2-pyrazinyl]imidazo[1,2-a]pyridin-7-yl]oxy]-, (2S)-;
2-Propanol, 1-[[3-[6-[(3R)-3-piperidinylamino]-2-pyrazinyl]imidazo[1,2-a]pyridin-7-yl]oxy]-, (2S)-;
1-Propanol, 2-[[3-[6-[(3R)-3-pyrrolidinylamino]-2-pyrazinyl]imidazo[1,2-a]pyridin-7-yl]oxy]-, (2R)-;
Ethanol, 2-[[3-[6-[(3R)-3-piperidinylamino]-2-pyrazinyl]imidazo[1,2-a]pyridin-7-yl]oxy]-;
2-Pyrazinamine, 6-imidazo[1,2-a]pyridin-3-yl-5-methyl-N-(3R)-3-pyrrolidinyl-;
1-Piperidinecarboxylic acid, 3-[[6-(6-chloroimidazo[1,2-a]pyridin-3-yl)-2-pyrazinyl]amino]-, 1,1-dimethylethyl ester, (3R)-;
1-Piperidinecarboxylic acid, 3-[[6-(6-methoxyimidazo[1,2-a]pyridin-3-yl)-2-pyrazinyl]amino]-, 1,1-dimethylethyl ester, (3R)-;
1-Piperidinecarboxylic acid, 3-[[6-[7-(aminocarbonyl)imidazo[1,2-a]pyridin-3-yl]-2-pyrazinyl]amino]-, 1,1-dimethylethyl ester, (3R)-;
1-Piperidinecarboxylic acid, 4-ethoxy-3-[(6-imidazo[1,2-a]pyridin-3-yl-2-pyrazinyl)amino]-, phenylmethyl ester, (3R,4R)-;
1-Piperidinecarboxylic acid, 3-[(6-imidazo[1,2-a]pyridin-3-yl-2-pyrazinyl)amino]-, 1,1-dimethylethyl ester, (3R)-;
1-Piperidinecarboxylic acid, 3-[(6-imidazo[1,2-a]pyridin-3-yl-2-pyrazinyl)amino]-, 1,1-dimethylethyl ester, (3S)-;
1-Pyrrolidinecarboxylic acid, 3-[(6-imidazo[1,2-a]pyridin-3-yl-2-pyrazinyl)amino]-, 1,1-dimethylethyl ester, (3R)-;
1-Pyrrolidinecarboxylic acid, 3-[(6-imidazo[1,2-a]pyridin-3-yl-2-pyrazinyl)amino]-4-methyl-, 1,1-dimethylethyl ester, (3R,4R)-;
1-Pyrrolidinecarboxylic acid, 3-[[6-[6-(trifluoromethyl)imidazo[1,2-a]pyridin-3-yl]-2-pyrazinyl]amino]-, 1,1-dimethylethyl ester, (3R)-;
1-Pyrrolidinecarboxylic acid, 3-[[6-[6-(1-hydroxy-1-methylethyl)imidazo[1,2-a]pyridin-3-yl]-2-pyrazinyl]amino]-4-methyl-, 1,1-dimethylethyl ester, (3R,4R)-;
1-Piperidinecarboxylic acid, 3-[(6-imidazo[1,2-a]pyridin-3-yl-3-methyl-2-pyrazinyl)amino]-, 1,1-dimethylethyl ester, (3R)-;
1-Piperidinecarboxylic acid, 3-[(5-cyano-6-imidazo[1,2-a]pyridin-3-yl-2-pyrazinyl)amino]-, 1,1-dimethylethyl ester, (3R)-;
Imidazo[1,2-a]pyridine-7-carboxylic acid, 3-[6-[[(3R)-1-[(1,1-dimethylethoxy)carbonyl]-3-piperidinyl]amino]-2-pyrazinyl]-, methyl ester;
1-Pyrrolidinecarboxylic acid, 2-(difluoromethyl)-4-[(6-imidazo[1,2-a]pyridin-3-yl-2-pyrazinyl)amino]-, 1,1-dimethylethyl ester, (2S,4R)-;
1-Piperidinecarboxylic acid, 4-fluoro-3-[(6-imidazo[1,2-a]pyridin-3-yl-2-pyrazinyl)amino]-, phenylmethyl ester, (3R,4S)-;
1-Piperidinecarboxylic acid, 4-fluoro-3-[(6-imidazo[1,2-a]pyridin-3-yl-2-pyrazinyl)amino]-, phenylmethyl ester, (3R,4R)-;
1-Piperidinecarboxylic acid, 3-[(6-imidazo[1,2-a]pyridin-3-yl-2-pyrazinyl)amino]-4-methoxy-, phenylmethyl ester, (3R,4R)-;
1-Piperidinecarboxylic acid, 5-[(6-imidazo[1,2-a]pyridin-3-yl-2-pyrazinyl)amino]-2-methyl-, phenylmethyl ester, (2S,5R)-;
1-Piperidinecarboxylic acid, 5-[(6-imidazo[1,2-a]pyridin-3-yl-2-pyrazinyl)amino]-2-methyl-, phenylmethyl ester, (2R,5R)-;
1-Piperidinecarboxylic acid, 3-[[6-[7-[2-(1-methylethoxy)ethoxy]imidazo[1,2-a]pyridin-3-yl]-2-pyrazinyl]amino]-, 1,1-dimethylethyl ester, (3R)-;
1-Piperidinecarboxylic acid, 3-[(6-imidazo[1,2-a]pyridin-3-yl-3-methoxy-2-pyrazinyl)amino]-, 1,1-dimethylethyl ester, (3R)-.

The above compounds are disclosed in WO2010/016005. Typically, the compounds of formula (I) exclude the compounds disclosed in WO2010/016005, including the salts, solvates and stereoisomers disclosed therein.

Typically, the compounds of formula (I) are other than
2-Pyrazinamine, 6-imidazo[1,2-a]pyridin-3-yl-N-(3-methoxyphenyl)-;
2-Pyrazinamine, N-(4-chlorophenyl)-6-(2-methylimidazo[1,2-a]pyridin-3-yl)-;
2-Pyrazinamine, N-(4-bromophenyl)-6-(2-methylimidazo[1,2-a]pyridin-3-yl)-;
2-Pyrazinamine, N-(4-methoxyphenyl)-6-(2-methylimidazo[1,2-a]pyridin-3-yl)-;
2-Pyrazinamine, N-(4-ethoxyphenyl)-6-(2-methylimidazo[1,2-a]pyridin-3-yl)-;
Ethanone, 1-[4-[[6-(2-methylimidazo[1,2-a]pyridin-3-yl)-2-pyrazinyl]amino]phenyl]-;
1,4-Benzenediamine, N1,N1-dimethyl-N4-[6-(2-methylimidazo[1,2-a]pyridin-3-yl)-2-pyrazinyl]-;
2-Pyrazinamine, N-(4-iodophenyl)-6-(2-methylimidazo[1,2-a]pyridin-3-yl)-;
2-Pyrazinamine, N-(3-bromophenyl)-6-(2-methylimidazo[1,2-a]pyridin-3-yl), hydrobromide.

The above compounds are disclosed in WO2010/002985. Typically, the compounds of formula (I) exclude the compounds disclosed in WO2010/002985, including the salts, solvates and stereoisomers disclosed therein.

Typically, in the compounds of formula (I), wherein m, X, Y, Z and R₁ to R₉ are as defined above, provided that when Y represents a nitrogen atom, and X represents a —CR₉ group, then;
when R₈ represents a 5- to 6-membered heterocyclyl group containing one nitrogen atom, which nitrogen atom is not bonded to the —Z—(CR₆R₇)ₘ— moiety, said nitrogen atom is substituted by a substituent other than a tert-butoxycarbonyl group or a benzyloxycarbonyl group; and
when R₈ represents a phenyl group, m is an integer from 1 to 3.

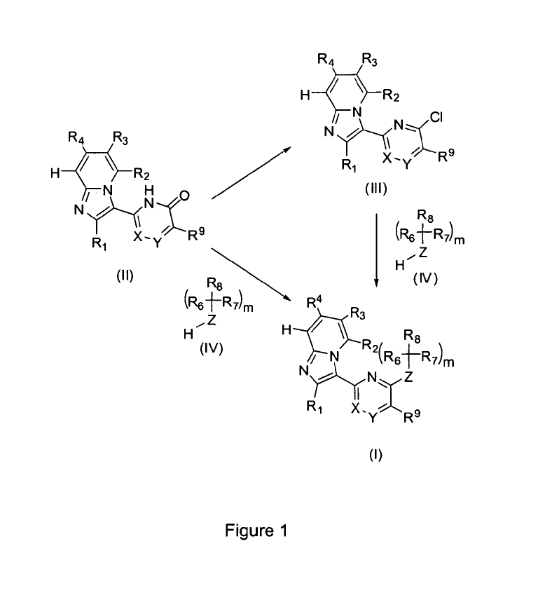

According to one embodiment of the present invention, compounds of general formula (I) may be prepared by the following synthetic route as illustrated in FIG. 1.

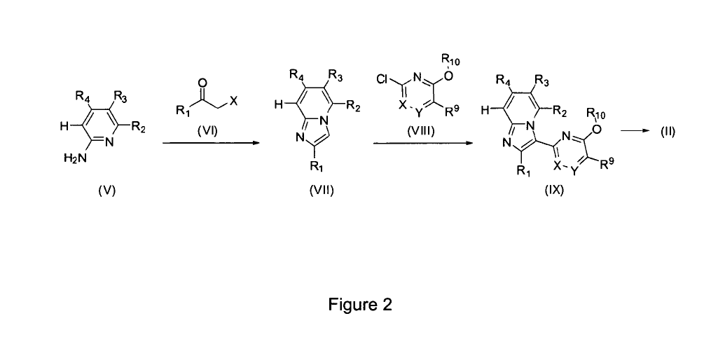

Compounds of formula (I) (where either X or Y is a nitrogen atom, the remaining atom being a CR₁₁ group) may be obtained directly from compounds of formula (II) by treatment of (II) with an appropriate activating agent such as benzotriazolyloxy-tris-(dimethylamino)phosphonium hexafluorophosphate in the presence of a suitable base such as 1,8-diazabicyclo[5.4.0]undec-7-ene at temperatures ranging from 25 to 80° C. in a suitable solvent such as N,N′-dimethylformamide in the presence of a nucleophile of type (IV), following the protocol as described in the literature (J. Org. Chem. 2007, 72 (26), 10194-10210). Alternatively, compounds of formula (II) may first be converted to chlorine-containing heteroaromatic compounds of formula (III), by treatment of (II) with a suitable chlorinating agent, for example phosphorous (V) oxychloride or phosphorous (V) chloride, at temperatures ranging from 25° C. to reflux. Compounds of formula (III) may then be converted to compounds of formula (I) by reaction with an appropriate nucleophile of formula (IV), such as an amine, in the presence of a base such as N,N′-diisopropylethylamine or triethylamine in a solvent such as N,N′-dimethylformamide, ethanol or tetrahydrofuran at temperatures ranging from ambient temperature to reflux with or without the use of microwave irradiation. In the particular case where Z═NR₅, compounds of formula (I) may be also prepared by reaction of chloroderivatives of formula (III) with amines of formula (IV) in the presence of a suitable catalyst such as tris(dibenzylideneacetone) dipalladium (0), a ligand such as 2′-(dicyclohexylphosphino)-N,N-dimethylbiphenyl-2-amine and a base, for example sodium tert-butoxide, in a solvent such as toluene at a temperature ranging from 80° C. to reflux. Compounds of general formula (II) may be obtained as illustrated in FIG. 2.

Treatment of 2-aminopyridines of formula (V) with haloderivatives of formula (VI), where X═Cl or Br, in the presence of a base, for example sodium hydrogen carbonate, in a suitable solvent such as acetonitrile or propan-2-ol at temperatures ranging from ambient temperature to reflux gives rise to imidazo[1,2-a]pyridines of formula (VII). Reaction of compounds of formula (VII) with chloroderivatives of formula (VIII) under palladium-catalyzed coupling conditions with a suitable catalyst such as tetrakis(triphenylphosphine)palladium (0) or the catalytically active species generated from palladium(II) acetate/triphenylphosphine in the presence of a base, for example potassium acetate or potassium carbonate, in a solvent such as dioxane, ethanol or N,N′-dimethylacetamide or a mixture thereof at temperatures ranging from 100-160° C. with or without the use of microwave irradiation, furnishes compounds of formula (IX). In the particular case where R₁₀=Me, treatment of methoxy derivatives of formula (IX) with a suitable reagent such as that generated from the reaction of trimethylsilylchloride and sodium iodide in a suitable solvent such as acetonitrile at reflux or with potassium hydroxide in ethanol under microwave heating, gives rise to desired compounds of formula (II).

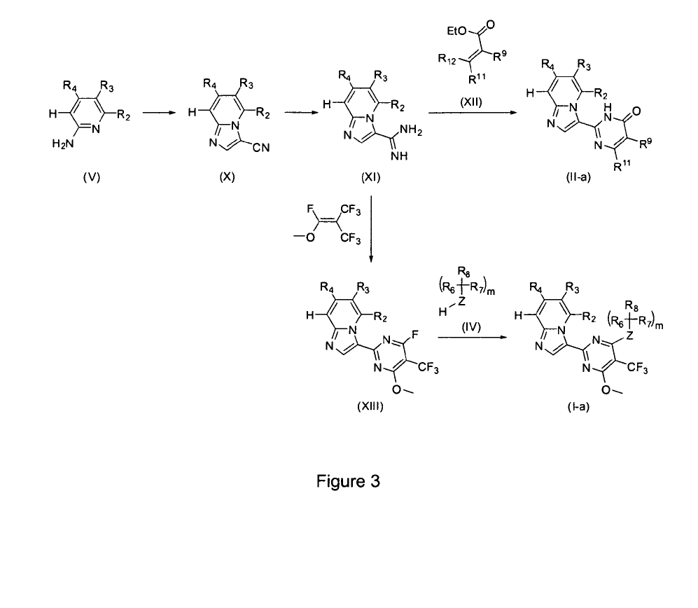

In the particular case of formula (II) where R₁═H, X═N and Y═CR₁₁, compounds of subformula (II-a) may be prepared by an alternative synthetic approach as shown in FIG. 3.

Imidazo[1,2-a]pyridine-3-carbonitriles of formula (X) may be prepared by treatment of 2-aminopyridines of formula (V) with 3-methoxyacrylonitrile in the presence of N-bromosuccinimide in a suitable solvent such a dioxane/water mixture at temperatures ranging from ambient temperature to reflux. Cyano derivatives of formula (X) may be converted to the corresponding amidines of formula (XI) by first formation of the corresponding imidate by reaction with alkoxy derivatives such as sodium methoxide or sodium ethoxide in a suitable alcoholic solvent at temperatures ranging from 0° C. to reflux followed by addition of ammonium chloride or ammonium hydroxide or by treatment with a suitable Lewis acid such as trimethylaluminium followed by addition of ammonium chloride in a suitable solvent such as toluene at temperatures ranging from 60° C. to reflux. Amidines of formula (XI) may be reacted with unsaturated esters of formula (XII) (where R₁₂ is —OH, —OMe, —OEt or —NMe₂) to give pyrimidin-4-ones of formula (II-a). Such reactions may be carried out in the presence of a suitable base such as sodium ethoxide, triethylamine or sodium carbonate in a solvent such as ethanol, isopropanol, tetrahydrofuran or water at temperatures ranging from ambient temperature to reflux with or without the use of microwave irradiation.

In the particular case of formula (I) where X═N, Y═C-OMe and R₉═CF₃, compounds of subformula (I-a) may be obtained from 4-fluoropyrimidines of formula (XIII) by treatment with a nucleophile of formula (IV), such as an amine, in a suitable solvent such as ethanol at ambient temperature. 4-Fluoropyrimidines of formula (XIII) may be prepared by reaction of amidines of formula (XI) with 1,3,3,3-tetrafluoro-1-methoxy-2-(trifluoromethyl)prop-1-ene in the presence of a suitable base such as sodium hydroxide at ambient temperature in a suitable mixture of solvents such as methylene chloride/water.

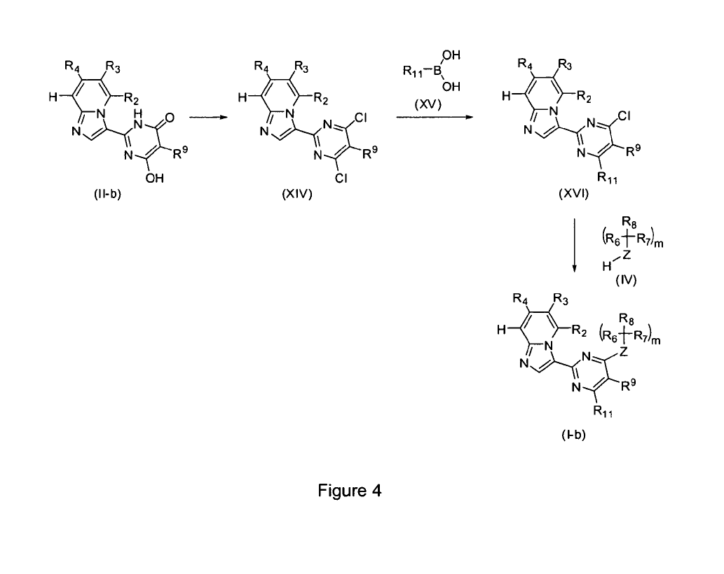

In another particular case, compounds of subformula (I-b) (derived from formula (I) where X═N and Y═CR₁₁) may be obtained as illustrated in FIG. 4.

Treatment of hydroxypyridones of formula (II-b) with a suitable chlorinating agent, for example phosphorous (V) oxychloride at temperatures ranging from 40° C. to reflux gives rise to dichloropyrimidines of formula (XIV). Compounds of formula (XIV) may be reacted with boronic acids of formula (XV) under Suzuki-Miyaura reaction conditions (Miyaura, N.; Suzuki, A. Chem. Rev. 1995, 95, 2457) to give compounds of formula (XVI). Such reactions may be catalyzed by a suitable palladium catalyst such as [1,1′-bis(diphenylphosphino)ferrocene]dichloropalladium (II) complex with dichloromethane (1:1), in a solvent such as 1,4-dioxane, in the presence of a base such as cesium carbonate, at temperatures ranging from 80° C. to reflux with or without the use of microwave irradiation. Chloropyridines of formula (XVI) may then be converted to compounds of subformula (I-b) by reaction with an appropriate nucleophile of formula (IV), such as an amine, in the presence of a base such as N,N′-diisopropylethylamine in a solvent such as N,N′-dimethylformamide at temperatures ranging from ambient temperature to 120° C.

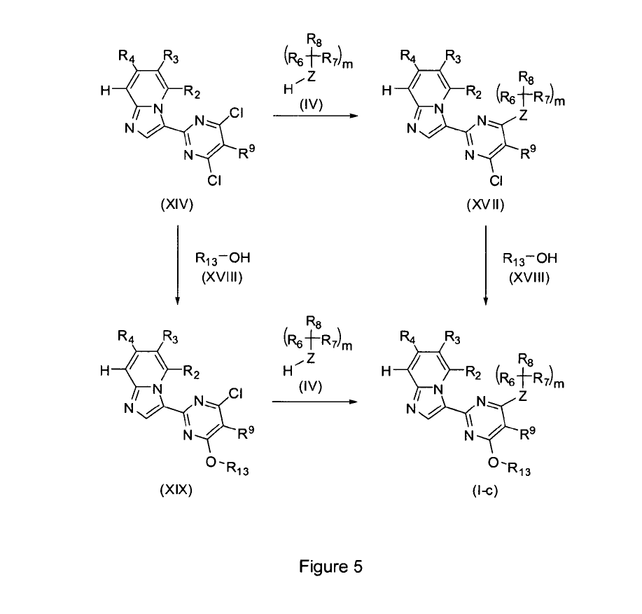

In yet another particular case, compounds of subformula (I-c) (derived from formula (I) where X═N and Y═C—OR₁₃) may be prepared as illustrated in FIG. 5.

Dichloroderivatives of formula (XIV) may be first converted to compounds of formula (XVII) by reaction with a nucleophile of formula (IV) in the presence of a base such as N,N′-diisopropylethylamine or triethylamine in a solvent such as N,N′-dimethylformamide, ethanol or tetrahydrofuran at temperatures ranging from ambient temperature to reflux. Treatment of alcohols of formula (XVIII) with a suitable base such as sodium hydride in a solvent such as tetrahydrofuran at temperatures ranging from 0° C. to ambient temperature give rise to intermediate alkoxy derivatives which may be reacted with chloropyrimidines of formula (XVII) in a suitable solvent such as tetrahydrofuran at temperatures ranging from ambient temperature to reflux to give compounds of subformula (I-c). Alternatively, compounds of subformula (I-c) may be prepared by reaction of dichloroderivatives of formula (XIV) with the alkoxy derivatives derived from alcohols of formula (XVIII) to give compounds of formula (XIX) followed by reaction of (XIX) with an appropriate nucleophile of formula (IV) in the presence of a base such as N,N′-diisopropylethylamine or triethylamine in a solvent such as N,N′-dimethylformamide, ethanol or tetrahydrofuran at temperatures ranging from ambient temperature to reflux.

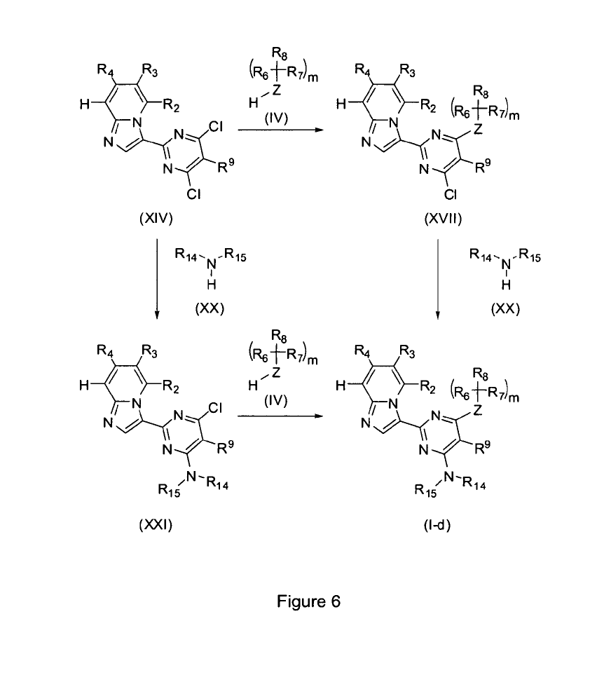

In yet another particular case, compounds of subformula (I-d) (derived from formula (I) where X═N and Y═C—NR₁₄R₁₅) may be prepared as shown in FIG. 6:

Reaction of dichloropyrimidines of formula (XIV) with amines of formula (XX) in the presence of a base such as triethylamine in a solvent such as ethanol at reflux gives rise to compounds of formula (XXI). Compounds of formula (XXI) may be transformed into compounds of subformula (I-d) by reaction with nucleophiles of formula (IV) in the presence of a base such as N,N′-diisopropylethylamine or triethylamine in a solvent such as N,N′-dimethylformamide, ethanol or tetrahydrofuran at temperatures ranging from ambient temperature to reflux. Alternatively, compounds of formula (I-d) may be prepared by treatment of chloropyrimidines of formula (XVII) with amines of formula (XX) in the presence of a base such as N,N′-diisopropylethylamine or cesium carbonate in a solvent such as N,N′-dimethylformamide or N-methylpirrolidone at temperatures ranging from 60 to 140° C. with or without the use of microwave irradiation.

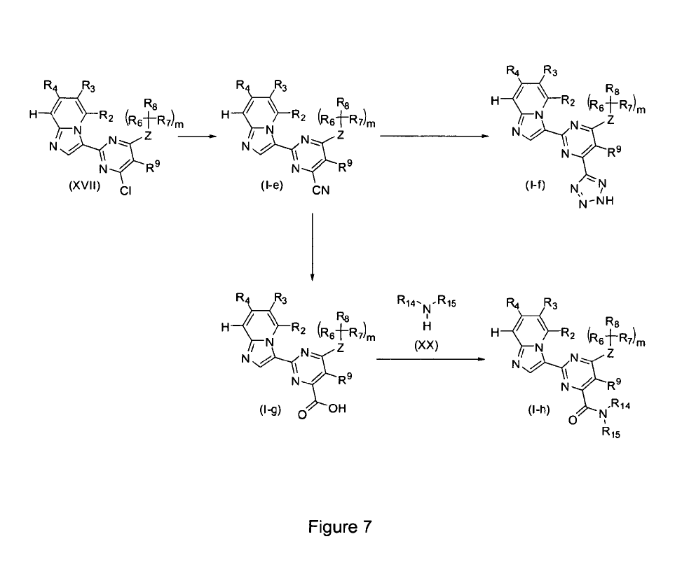

In yet other particular cases, compounds of subformulae (I-e), (I-f), (I-g) and (I-h) where X═N and Y═C—CN, C-tetrazole, C—COOH, C—CONR₁₄R₁₅ respectively, may be prepared as illustrated in FIG. 7.

Treatment of chloropyrimidines of formula (XVII) with a source of cyanide ion, such as zinc (II) cyanide in the presence of a palladium catalyst such tetrakis(triphenylphosphine)palladium (0) in a suitable solvent such as N,N′-dimethylformamide at temperatures ranging from 80 to 130° C. furnishes cyano derivatives of subformula (I-e). Nitriles of subformula (I-e) may be converted into tetrazoles of subformula (I-f) by reaction with azidotrimethylstannane in a suitable solvent such as toluene at reflux. Alternatively, treatment of cyano derivatives of subformula (I-e) with a suitable base such as sodium hydroxide in a mixture of solvents such as methanol/water at 100° C. in a sealed tube under microwave irradiation gives rise to carboxylic acids of subformula (I-g). Compounds of subformula (I-g) may be converted into amides of subformula (I-h) by treatment with an appropriate activating agent such as 1-hydroxybenzotriazole hydrate/N-(3-dimethylaminopropyl)-N′-ethylcarbodiimide hydrochloride in a solvent such as N,N′-dimethylformamide at ambient temperature in the presence of an amine of formula (XX).

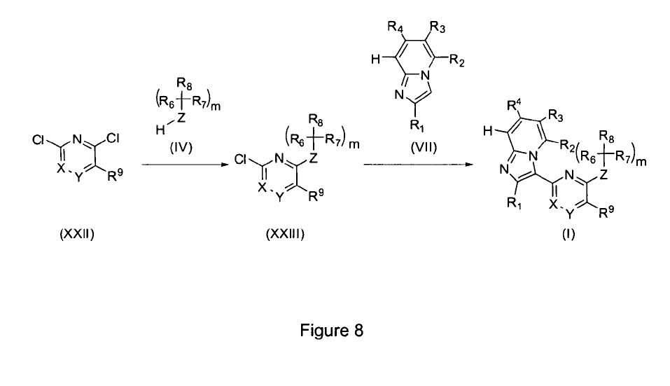

Compounds of general formula (I) may be prepared via yet another synthetic pathway, as shown in FIG. 8.

Treatment of dichloro heteroaromatic compounds of formula (XXII) with nucleophiles of formula (IV) in the presence of a base, such as N,N′-diisopropylethylamine, in an aprotic solvent such as N,N′-dimethylformamide at temperatures ranging from ambient temperature to reflux gives rises to compounds of formula (XXIII). Reaction of compounds of formula (XXIII) with aforementioned intermediates of formula (VII) using a suitable catalyst such as tetrakis(triphenylphosphine)palladium (0) or the catalytically active species generated from palladium(II) acetate/triphenylphosphine in the presence of a base, for example potassium acetate or potassium carbonate, in a solvent such as dioxane, ethanol or N,N′-dimethylacetamide or a solvent mixture thereof at temperatures ranging from 100-160° C. with or without the use of microwave irradiation gives rise to desired compounds of formula (I).

In the particular case of compounds of formula (I) where R₃ or R₉ is a halogen atom, further reaction with a suitable boronic acid or boronate under Suzuki-Miyaura reaction conditions (Miyaura, N.; Suzuki, A. Chem. Rev. 1995, 95, 2457) gives rise to compounds or formula (I) where the corresponding halogen atom at R₃ or R₉ has been replaced with an aliphatic, aromatic or heteroaromatic residue. Such reactions may be catalyzed by a suitable palladium catalyst such as tetrakis(triphenylphospino)palladium (0), in a solvent such as 1,4-dioxane, in the presence of a base such as potassium carbonate, at a temperature ranging from 80-110° C. Alternatively, compounds of formula (I), where R₃ or R₉ is a halogen atom, may be further reacted with a suitable amine in the presence of a palladium catalyst to give compounds of formula (I) where the corresponding halogen atom at R₃ or R₉ has been replaced with an amine residue. Such reactions may be catalyzed by a suitable palladium catalyst generated from, for example, tris(dibenzylideneacetone)dipalladium (0) and biphenyl-2-yl-di-tert-butylphosphine, in a solvent such as toluene, in the presence of a base such as sodium tert-butoxide, at a temperature ranging from 80-150° C. with or without the use of microwave irradiation.

In another particular case, compounds of formula (I), where R₉ is a hydrogen atom, may be further reacted by treatment with a halogenating agent such as N-bromosuccinimide or molecular bromine in a solvent such as acetic acid or N,N′-dimethylformamide to give compounds of formula (I), where R₉ is now a halogen residue.

In yet another particular case, compounds of formula (I), where Z=NR₅ and R₅ is a hydrogen atom, may undergo further reaction with a suitable base such as sodium hydride in a solvent such as N,N′-dimethylformamide followed by the addition of an alkylating agent, such as methyl iodide at temperatures ranging from 0° C. to reflux, to furnish compounds of formula (I), where R₅ is now an alkyl group.

In yet another particular case, compounds of formula (I), in which the residue at R₆, R₇, R₈, R₉ or R₁₁ (in the particular case where Y═CR₁₁) contains, in part, an amine moiety functionalized with an appropriate protecting group such as tert-butoxycarbonyl (BOC), benzyloxycarbonyl (CBZ) or p-methoxybenzyl (PMB), may be deprotected at the amine moiety under standard conditions (Greene's Protective Groups in Organic Synthesis, ISBN: 0471697540). The corresponding free amine may then be further functionalized under standard conditions to give the corresponding amides, sulphonamides, ureas, and N-alkylated and arylated amines.

In yet another particular case, compounds of formula (I), in which the residue at R₆, R₇ R₈, R₉ or R₁₁ (in the particular case where Y═CR₁₁) contains a carboxylic acid moiety functionalized with an appropriate protecting group such as methyl ester, may be deprotected at the carboxylic acid moiety under standard conditions (Greene's Protective Groups in Organic Synthesis, ISBN: 0471697540). The corresponding carboxylic acid may then be further functionalized under standard conditions to give the corresponding amides.

In yet another particular case, compounds of formula (I), in which the residue at R₉ is a nitro moiety, may be reduced to the corresponding primary amines by treatment with an appropriate reducing agent such as tin (II) chloride in a solvent such as ethanol at temperatures ranging from 20-100° C. or hydrogen gas at atmospheric pressure using a suitable catalyst such as palladium or platinum on carbon in a solvent such as ethanol or methanol at ambient temperature.

The syntheses of the compounds of the invention and of the intermediates for use therein are illustrated by the following Examples (1-186) (including Preparation Examples (Preparations 1-97)) and are given in such an order to provide a person skilled in the art with a sufficiently clear and complete explanation of the present invention, but should not be considered as limiting of the essential aspects of its subject, as set out in the preceding portions of this description.

### Preparation 1

### Imidazo[1,2-a]pyridine-6-carbonitrile

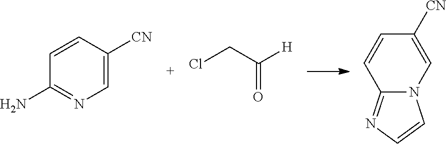

A solution of 6-aminonicotinonitrile (10 g, 80 mmol) in acetonitrile (300 mL) was treated with a 50% aqueous solution of 2-chloroacetaldehyde (26.4 mL, 210 mmol). The mixture was stirred and heated to reflux. After 6 hours, the mixture was cooled to room temperature. The mixture was concentrated to low bulk (approx. 100 mL), treated with saturated aqueous sodium hydrogencarbonate solution to neutral pH, and then extracted with dichloromethane (2×300 mL). The organic layer was dried (MgSO₄) and evaporated and the residue was stirred with diethyl ether (200 mL), filtered and dried in vacuum to give the title compound (22.54 g, 94%) as a pale brown solid.

LRMS (m/z): 144 (M+1)⁺.

¹H-NMR δ (CDCl₃): 7.29 (dd, 1H), 7.71 (d, 1H), 7.73 (d, 1H), 7.80 (d, 1H), 8.61-8.62 (m, 1H).

### Preparation 2

### 6-Fluoroimidazo[1,2-a]pyridine

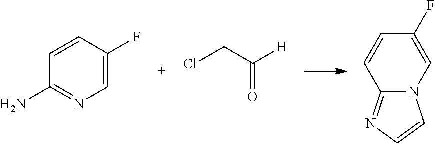

Obtained as a brown solid (93%) from 5-fluoropyridin-2-amine and 2-chloroacetaldehyde (50% in water) following the experimental procedure as described in Preparation 1.

LRMS (m/z): 137 (M+1)⁺.

¹H-NMR δ (CDCl₃): 7.12 (m, 1H), 7.57-7.67 (m, 2H), 7.70 (bs, 1H), 8.04-8.13 (m, 1H).

### Preparation 3

### 6-Chloroimidazo[1,2-a]pyridine

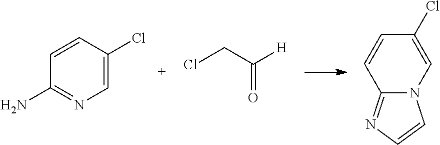

Obtained as a brown solid (71%) from 5-chloropyridin-2-amine and 2-chloroacetaldehyde (50% in water) following the experimental procedure as described in Preparation 1 followed by purification of the crude product by flash chromatography (1:1 hexanes/ethyl acetate).

LRMS (m/z): 153 (M+1)⁺.

¹H-NMR δ (CDCl₃): 7.13 (d, 1H), 7.53-7.60 (m, 2H), 7.66 (s, 1H), 8.18 (s, 1H).

### Preparation 4

### 3-(4-Hydroxypyrimidin-2-yl)imidazo[1,2-a]pyridine-6-carbonitrile

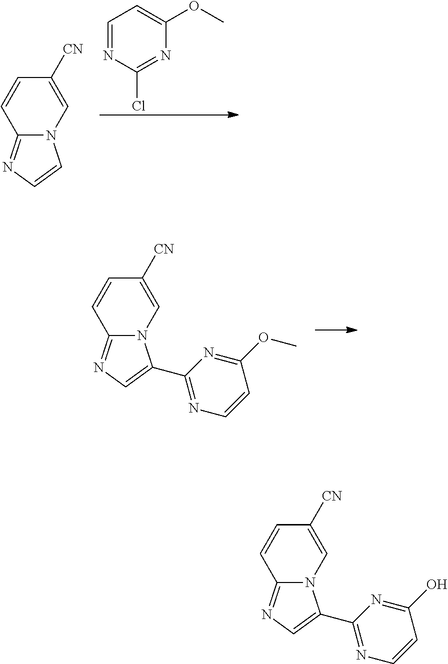

### a) 3-(4-Methoxypyrimidin-2-yl)imidazo[1,2-a]pyridine-6-carbonitrile

Nitrogen was bubbled for five minutes into a stirred mixture of 2-chloro-4-methoxypyrimidine (1.14 g, 7.9 mmol), imidazo[1,2-a]pyridine-6-carbonitrile (Preparation 1, 0.75 g, 5.2 mmol), potassium carbonate (1.45 g, 10.5 mmol) and triphenylphosphine (0.55 g, 2.1 mmol) in 1,4-dioxane (10 mL) and ethanol (5 mL) in a microwave vessel. Then palladium (II) acetate (0.24 g, 1.1 mmol) was added and the vessel was sealed and subjected to microwave irradiation for 2 hours at 150° C. The reaction was repeated 5 further times under the same conditions and the six experiments were combined and the solvent was evaporated. The residue was taken up in a mixture of ethyl acetate and water and filtered to remove an insoluble black solid. The organic layer was separated and extracted with 2M aqueous hydrochloric acid (3×80 mL). The combined aqueous layer was washed with diethyl ether and then treated with solid sodium hydrogencarbonate until a pH of approximately 6 was reached. The solid that formed was filtered and dried in vacuum to give a first crop of the title compound (2.65 g). The insoluble black solid was treated with 2M aqueous hydrochloric acid (2×60 mL) and filtered. The aqueous solution was treated with solid sodium hydrogencarbonate until a pH of approximately 6 was reached. The solid that formed was filtered and dried in vacuum to give a second crop of the title compound (0.77 g). Total yield=3.42 g (43%).

LRMS (m/z): 252 (M+1)⁺.

¹H-NMR δ (DMSO-d₆): 4.08 (s, 3H), 6.88 (d, 1H), 7.76 (dd, 1H), 7.95 (dd, 1H), 8.63 (s, 1H), 8.67 (d, 1H), 10.39 (bs, 1H).

### b) 3-(4-Hydroxypyrimidin-2-yl)imidazo[1,2-a]pyridine-6-carbonitrile

Sodium iodide (11.8 g, 78.6 mmol) was added to a stirred suspension of 3-(4-methoxypyrimidin-2-yl)imidazo[1,2-a]pyridine-6-carbonitrile (Preparation 4a, 3.3 g, 13.1 mmol) in acetonitrile (135 mL). Chlorotrimethylsilane (9.9 mL, 78.0 mmol) was added and the mixture was heated in a sealed tube at 80° C. for 24 hours and then cooled to ambient temperature. The solvent was removed under reduced pressure and the residue was suspended in water. Saturated aqueous sodium thiosulphate solution was added until the dark colour faded and a pale brown solid formed. The solid was filtered, washed with further saturated sodium thiosulphate solution and water and dried in vacuum to give the title compound (3.2 g, 99%) as a pale brown solid.

LRMS (m/z): 238 (M+1)⁺, 236 (M−1)⁻.

¹H-NMR δ (DMSO-d₆): 6.42 (d, 1H), 7.82 (d, 1H), 7.97 (d, 1H), 8.27 (d, 1H), 8.78 (s, 1H), 10.43 (s, 1H).

### Preparation 5

3-{4-[(3R)-Piperidin-3-ylamino]pyrimidin-2-yl}imidazo[1,2-a]pyridine-6-carbonitrile

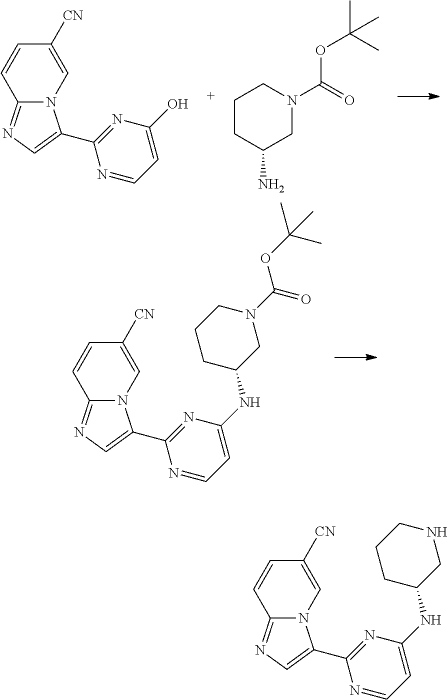

### a) tert-Butyl-(3R)-3-{[2-(6-cyanoimidazo[1,2-a]pyridin-3-yl)pyrimidin-4-yl]amino}piperidine-1-carboxylate

(Benzotriazol-1-yloxy)tris(dimethylamino)phosphonium hexafluorophosphate (0.480 g, 1.09 mmol) and 1,8-diazabicyclo[5.4.0]undec-7-ene (0.187 mL, 1.25 mmol) were added sequentially to a stirred solution of 3-(4-hydroxypyrimidin-2-yl)imidazo[1,2-a]pyridine-6-carbonitrile (Preparation 4b, 0.2 g, 0.84 mmol) in N,N′-dimethylformamide (3 mL). After stirring for 15 minutes at ambient temperature, tert-butyl (3R)-3-aminopiperidine-1-carboxylate (0.418 g, 2.09 mmol) was added dropwise and the mixture was stirred for a further 16 hours at ambient temperature. After addition of water and extraction with ethyl acetate, the organic layer was washed with water, 4% aqueous sodium hydrogen carbonate solution, brine, dried (MgSO₄) and evaporated. The residue was purified by flash chromatography (99:1 to 98:2 dichloromethane/methanol) to obtain the title compound (70%) as a yellow solid.

LRMS (m/z): 420 (M+1)⁺.

¹H-NMR δ (DMSO-d₆): 0.92-1.65 (m, 15H), 3.66-4.04 (m, 3H), 6.29-6.69 (m, 1H), 7.91 (d, 2H), 8.12-8.33 (m, 1H), 8.40-8.68 (m, 1H), 10.28-10.61 (m, 1H).

### b) 3-{4-[(3R)-Piperidin-3-ylamino]pyrimidin-2-yl}imidazo[1,2-a]pyridine-6-carbonitrile

Trifluoroacetic acid (1.0 mL, 12 mmol) was added dropwise to a solution of tert-butyl-(3R)-3-{[2-(6-cyanoimidazo[1,2-a]pyridin-3-yl)pyrimidin-4-yl]amino}piperidine-1-carboxylate (Preparation 5a, 0.25 g, 0.6 mmol) in dichloromethane (5 mL) and the mixture was stirred at ambient temperature for one hour. After the reaction was complete, water was added and the mixture was neutralized with solid sodium hydrogen carbonate and then extracted with dichloromethane. The organic layer was dried (MgSO₄) and evaporated and the residue was purified by flash chromatography (96:4 dichloromethane/methanol) obtaining the title compound (99%) as a pale yellow solid.

LRMS (m/z): 320 (M+1)⁺.

¹H-NMR δ (DMSO-d₆): 1.08-1.87 (m, 4H), 2.01 (bs, 1H), 2.55-3.35 (m, 4H), 4.13 (bs, 1H), 6.45 (d, 1H), 7.49-7.79 (m, 2H), 7.91 (d, 1H), 8.21 (bs, 1H), 8.55 (bs, 1H), 10.44 (bs, 1H).

### Preparation 6

### (R)-3-(4-(Methyl(piperidin-3-yl)amino)pyrimidin-2-yl)imidazo[1,2-a]pyridine-6-

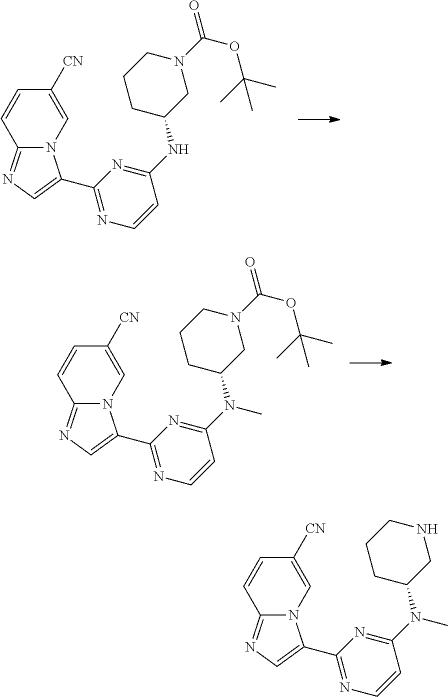 [SMILES: CC(C)(C)OC(=O)N1CCC[C@@H](Nc2ccnc(-c3cnc4ccc(C#N)cn34)n2)C1 ; CN(c1ccnc(-c2cnc3ccc(C#N)cn23)n1)[C@@H]1CCCN(C(=O)OC(C)(C)C)C1 ; CN(c1ccnc(-c2cnc3ccc(C#N)cn23)n1)[C@@H]1CCCNC1]

### a) tert-Butyl-3-[[2-(6-cyano-1,8a-dihydroimidazo[1,2-a]pyridin-3-yl)pyrimidin-4-yl](methyl)amino]piperidine-1-carboxylate

Sodium hydride (60% dispersion in mineral oil, 0.31 g, 7.75 mmol) was added portionwise to a stirred solution of (R)-tert-butyl 3-(2-(6-cyanoimidazo[1,2-a]pyridin-3-yl)pyrimidin-4-ylamino)piperidine-1-carboxylate (Preparation 5a, 1.67 g, 3.9 mmol) in N,N′-dimethylformamide (15 mL). When hydrogen evolution had ceased, methyl iodide (0.48 mL, 7.7 mmol) was added and the reaction mixture was stirred overnight at ambient temperature. The solvent was removed and the residue was partitioned between ethyl acetate and water. The organic layer was dried (MgSO₄) and evaporated and the residue was purified by reverse phase chromatography (C-18 silica from Waters, water/1:1 acetonitrile-methanol as eluents [0.1% v/v formic acid buffered] 0% to 100%) to give the title compound (0.26 g, 16%) as an oil.

LRMS (m/z): 434 (M+1)⁺.

¹H-NMR δ (CDCl₃): 1.39-1.65 (m, 9H), 1.64-2.08 (m, 4H), 2.64-2.89 (m, 1H), 3.09 (bs, 3H), 4.18 (bs, 2H), 6.41-6.48 (m, 1H), 7.40 (dd, 1H), 7.80 (d, 1H), 8.32 (d, 1H), 8.65 (bs, 1H), 10.55 (bs, 1H).

### b) (R)-3-(4-(Methyl(piperidin-3-yl)amino)pyrimidin-2-yl)imidazo[1,2-a]pyridine-6-carbonitrile

Obtained as a solid (60%) from (R)-tert-butyl 3-((2-(6-cyanoimidazo[1,2-a]pyridin-3-yl)pyrimidin-4-yl)(methyl)amino)piperidine-1-carboxylate (Preparation 6a) following the experimental procedure as described in Preparation 5b.

LRMS (m/z): 334 (M+1)⁺.

### Preparation 7

### 3-{4-[(3S)-Piperidin-3-ylamino]pyrimidin-2-yl}imidazo[1,2-a]pyridine-6-carbonitrile

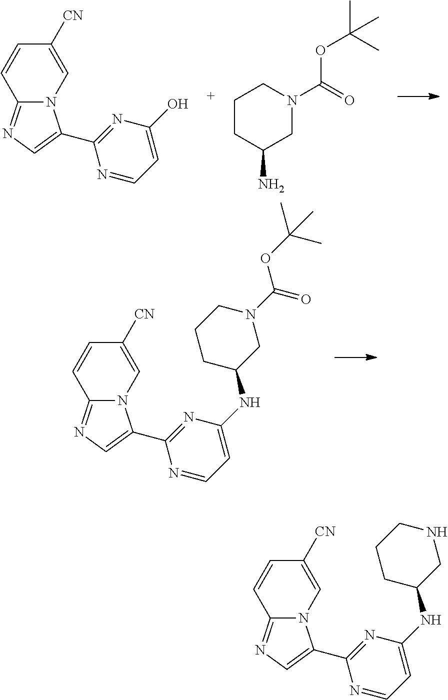

### a) tert-Butyl-(3S)-3-{[2-(6-cyanoimidazo[1,2-a]pyridin-3-yl)pyrimidin-4-yl]amino}piperidine-1-carboxylate

Obtained as a brown solid (66%) from 3-(4-hydroxypyrimidin-2-yl)imidazo[1,2-a]pyridine-6-carbonitrile (Preparation 4b) and tert-butyl (3S)-3-aminopiperidine-1-carboxylate following the experimental procedure as described in Preparation 5a. After the reaction was complete the mixture was partitioned between water and ethyl acetate. The organic layer was washed with water, saturated aqueous sodium hydrogen carbonate solution, brine, dried (MgSO₄) and evaporated and the residue was purified by flash chromatography (98:2 to 96:4 dichloromethane/methanol).

LRMS (m/z): 420 (M+1)⁺.

¹H-NMR δ (CDCl₃): 1.60 (s, 9H), 6.30 (d, 1H), 7.41 (d, 1H), 7.78 (d, 1H), 8.30 (d, 1H), 8.61 (s, 1H), 10.55 (s, 1H).

### b) 3-{4-[(3S)-Piperidin-3-ylamino]pyrimidin-2-yl}imidazo[1,2-a]pyridine-6-carbonitrile

Obtained as a solid (98%) from tert-butyl (3S)-3-{[2-(6-cyanoimidazo[1,2-a]pyridin-3-yl)pyrimidin-4-yl]amino}piperidine-1-carboxylate (Preparation 7a) following the experimental procedure as described in Preparation 5b

LRMS (m/z): 320 (M+1)⁺.

¹H-NMR δ (CDCl₃): 2.91 (s, 2H), 3.23 (dt, 1H), 5.70 (s, 1H), 6.40-6.12 (m, 1H), 7.45-7.30 (m, 1H), 7.88-7.67 (m, 1H), 8.25 (s, 1H), 8.59 (d, 1H), 10.55 (s, 1H).

### Preparation 8

### 2-(6-Fluoroimidazo[1,2-a]pyridin-3-yl)pyrimidin-4-ol

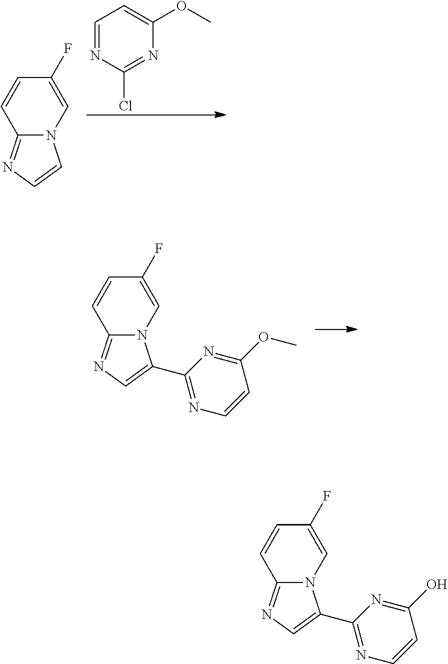

### a) 6-Fluoro-3-(4-methoxypyrimidin-2-yl)imidazo[1,2-a]pyridine

2-Chloro-4-methoxypyrimidine (7.0 g, 48 mmol) and 6-fluoroimidazo[1,2-a]pyridine (Preparation 2, 4.40 g, 32 mmol) were reacted according to the experimental procedure as described in Preparation 4a. Ethyl acetate was added to the crude reaction mixture and the suspension was filtered through Celite®. The filtrate was washed with water and extracted several times with 2M aqueous hydrochloric acid. The combined aqueous layer was washed with diethyl ether and then neutralized with 8M aqueous sodium hydroxide solution. The product was extracted into chloroform and the organic phase was dried (MgSO₄) and evaporated to afford the title compound (90%) as a grey solid, which was used without further purification.

LRMS (m/z): 245 (M+1)⁺.

¹H-NMR δ (CDCl₃): 4.11 (s, 3H), 6.58 (d, 1H), 7.60 (q, 1H), 7.71 (dd, 1H), 8.49 (d, 1H), 8.61 (s, 1H), 9.98 (dd, 1H).

### b) 2-(6-Fluoroimidazo[1,2-a]pyridin-3-yl)pyrimidin-4-ol

To a solution of 6-fluoro-3-(4-methoxypyrimidin-2-yl)imidazo[1,2-a]pyridine (Preparation 8a, 1.5 g, 6.1 mmol) in ethanol (10 mL) was added a 35% aqueous solution of potassium hydroxide (9.8 mL, 61.3 mmol). The reaction mixture was subjected to microwave irradiation for 1 hour at 120° C. The organic solvent was removed under reduced pressure and the aqueous phase was neutralized with 5M hydrochloric acid. The precipitate was filtered, washed with water and dried to afford the title compound (70%) as a white solid, which was used without further purification.

LRMS (m/z): 231 (M+1)⁺.

¹H-NMR δ (DMSO-d₆): 6.31 (s, 1H), 7.77 (d, 1H), 8.17 (s, 1H), 8.73 (bs, 1H), 9.94 (bs, 1H), 12.75 (s, 1H).

### Preparation 9

### (R)-2-(6-Fluoroimidazo[1,2-a]pyridin-3-yl)-N-(piperidin-3-yl)pyrimidin-4-amine

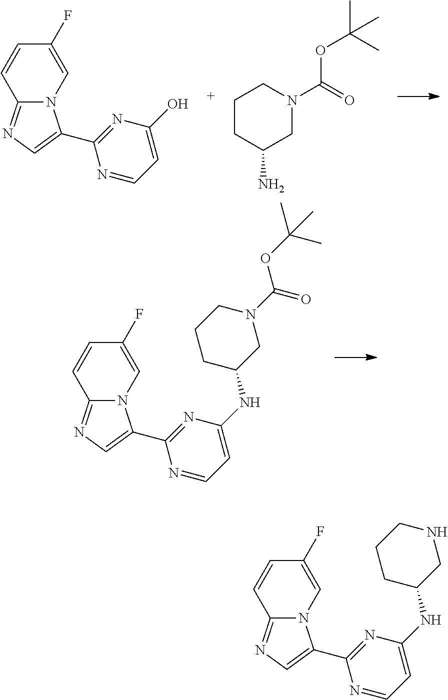

### a) tert-Butyl-(3R)-3-({[2-(6-fluoroimidazo[1,2-a]pyridin-3-yl)pyrimidin-4-yl]oxy}amino)piperidine-1-carboxylate

Obtained as a yellow solid (65%) from 2-(6-fluoroimidazo[1,2-a]pyridin-3-yl)pyrimidin-4-ol (Preparation 8b) and (R)-tert-butyl 3-aminopiperidine-1-carboxylate following the experimental procedure as described in Preparation 5a. The crude product was purified by reverse phase chromatography (C-18 silica from Waters, water/1:1 acetonitrile-methanol as eluents [0.1% v/v formic acid buffered] 0% to 100%).

LRMS (m/z): 413 (M+1)⁺.

¹H-NMR δ (CDCl₃): 1.44 (s, 9H), 2.13-1.53 (m, 4H), 3.65-3.23 (m, 4H), 5.14 (s, 1H), 6.24 (d, 1H), 7.23 (ddd, 1H), 7.68 (dd, 1H), 8.27 (d, 1H), 8.54 (s, 1H), 9.99 (s, 1H).

### b) (R)-2-(6-Fluoroimidazo[1,2-a]pyridin-3-yl)-N-(piperidin-3-yl)pyrimidin-4-amine

Obtained as a yellow solid (70%) from (R)-tert-butyl 3-(2-(6-fluoroimidazo[1,2-a]pyridin-3-yl)pyrimidin-4-ylamino)piperidine-1-carboxylate (Preparation 9a) following the experimental procedure as described in Preparation 5b.

LRMS (m/z): 313 (M+1)⁺.

¹H-NMR δ (CDCl₃): 1.65 (dd, 2H), 1.90-1.71 (m, 3H), 2.00-1.89 (m, 2H), 2.88 (d, 3H), 5.57 (d, 1H), 6.22 (d, 1H), 7.22 (ddd, 1H), 7.66 (dd, 1H), 8.22 (d, 1H), 8.53 (s, 1H), 9.98 (dd, 1H).

### Preparation 10

### 1-{[2-((3R)-3-{[2-(6-Fluoroimidazo[1,2-a]pyridin-3-yl)pyrimidin-4-yl]amino}piperidin-1-yl)-2-methylpropanoyl]oxy}pyrrolidine-2,5-dione

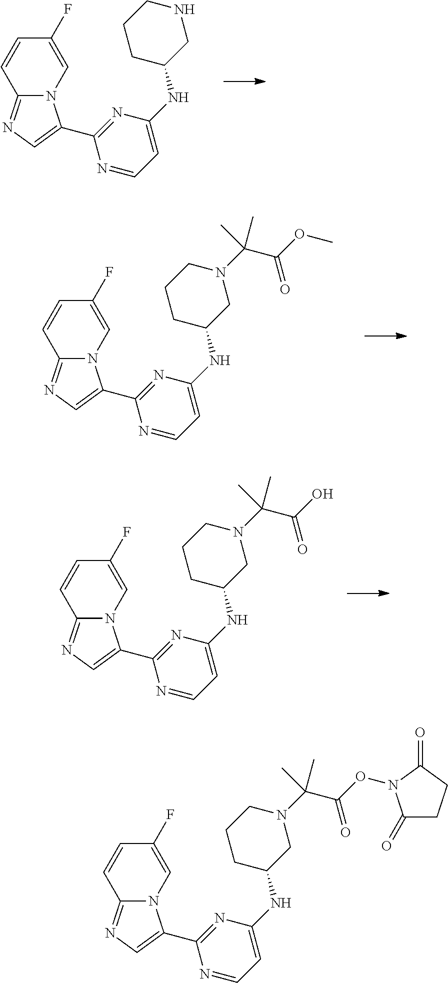 [SMILES: Fc1ccc2ncc(-c3nccc(N[C@@H]4CCCNC4)n3)n2c1 ; COC(=O)C(C)(C)N1CCC[C@@H](Nc2ccnc(-c3cnc4ccc(F)cn34)n2)C1 ; CC(C)(C(=O)O)N1CCC[C@@H](Nc2ccnc(-c3cnc4ccc(F)cn34)n2)C1 ; CC(C)(C(=O)ON1C(=O)CCC1=O)N1CCC[C@@H](Nc2ccnc(-c3cnc4ccc(F)cn34)n2)C1]

### a) Methyl 2-((3R)-3-{[2-(6-fluoroimidazo[1,2-a]pyridin-3-yl)pyrimidin-4-yl]amino}piperidin-1-O-2-methylpropanoate

A solution of (R)-2-(6-fluoroimidazo[1,2-a]pyridin-3-yl)-N-(piperidin-3-yl)pyrimidin-4-amine (Preparation 9b, 0.100 g, 0.32 mmol), ethyl 2-bromo-2-methylpropanoate (0.137 g, 0.70 mmol) and potassium carbonate (0.066 g, 0.48 mmol) in N,N′-dimethylformamide (1 mL) was stirred and heated to 80° C. After 16 hours, water was added to the reaction mixture and the mixture was extracted with dichloromethane. The organic layer was dried (MgSO₄) and evaporated and the residue was purified by reverse phase chromatography (C-18 silica from Waters, water/1:1 acetonitrile-methanol as eluents [0.1% v/v formic acid buffered] 0% to 100%) to give the title compound (0.090 g, 66%) as a solid.

LRMS (m/z): 413 (M+1)⁺.

### b) 2-((3R)-3-{[2-(6-Fluoroimidazo[1,2-a]pyridin-3-yl)pyrimidin-4-yl]amino}piperidin-1-yl)-2-methylpropanoic acid

Potassium hydroxide (0.028 g, 0.42 mmol) was added to a stirred solution of (R)-ethyl 2-(3-(2-(6-fluoroimidazo[1,2-a]pyridin-3-yl)pyrimidin-4-ylamino)piperidin-1-yl)-2-methylpropanoate (Preparation 10a, 0.090 g, 0.21 mmol) in methanol (1 mL). The reaction mixture was stirred at room temperature overnight and then heated to 50° C. and stirred a further 24 hours. The mixture was neutralized with 2M aqueous hydrochloric acid and then extracted with chloroform. The organic layer was dried (MgSO₄) and evaporated to give the title compound (0.050 g, 60%) as an oil which was used without further purification.

LRMS (m/z): 399 (M+1)⁺.

### c) 1-{[2-((3R)-3-{[2-(6-Fluoroimidazo[1,2-a]pyridin-3-yl)pyrimidin-4-yl]amino}piperidin-1-yl)-2-methylpropanoyl]oxy}pyrrolidine-2,5-dione

N-Hydroxysuccinimide (0.016 g, 0.14 mmol) and 1,3-diisopropylcarbodiimide (0.022 mL, 0.14 mmol) were added sequentially to a cooled (ice-bath), stirred solution of (R)-2-(3-(2-(6-fluoroimidazo[1,2-a]pyridin-3-yl)pyrimidin-4-ylamino)piperidin-1-yl)-2-methylpropanoic acid (Preparation 10b, 0.050 g, 0.13 mmol) in N,N′-dimethylformamide (1 mL). After stirring overnight at ambient temperature, water was added to the reaction mixture and the mixture was extracted with dichloromethane. The organic layer was dried (MgSO₄) and evaporated to give the crude title compound (0.050 g) as an oil which was used without further purification.

LRMS (m/z): 496 (M+1)⁺.

### Preparation 11

### (R)-2-(6-Fluoroimidazo[1,2-a]pyridin-3-yl)-N-methyl-N-(piperidin-3-yl)pyrimidin-4-amine

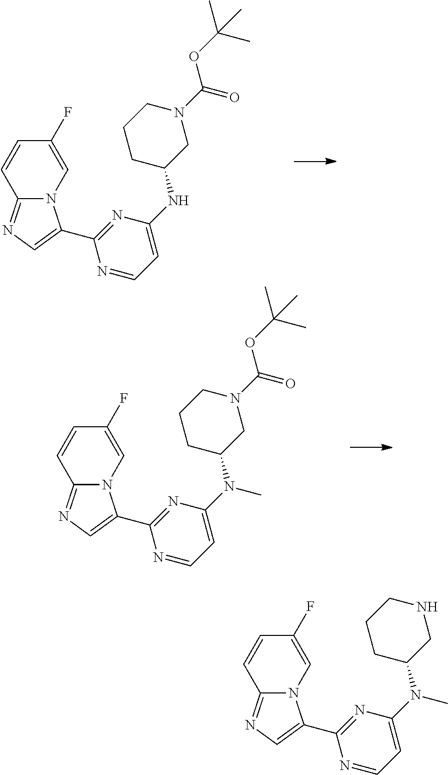 [SMILES: CC(C)(C)OC(=O)N1CCC[C@@H](Nc2ccnc(-c3cnc4ccc(F)cn34)n2)C1 ; CN(c1ccnc(-c2cnc3ccc(F)cn23)n1)[C@@H]1CCCN(C(=O)OC(C)(C)C)C1 ; CN(c1ccnc(-c2cnc3ccc(F)cn23)n1)[C@@H]1CCCNC1]

### a) (R)-tert-Butyl 3-02-(6-fluoroimidazo[1,2-a]pyridin-3-yl)pyrimidin-4-yl)(methyl)amino)piperidine-1-carboxylate

Obtained from tert-butyl (3R)-3-({[2-(6-fluoroimidazo[1,2-a]pyridin-3-yl)pyrimidin-4-yl]oxy}amino)piperidine-1-carboxylate (Preparation 9a) and methyl iodide following the experimental procedure as described in Preparation 6a. The crude product was purified by flash chromatography (0-100% ethyl acetate in hexane) to give the title compound (0.52 g, 55%) as an oil.

LRMS (m/z): 427 (M+1)⁺.

¹H-NMR δ (CDCl₃): 1.46 (bs, 9H), 1.67 (bs, 1H), 1.72-1.93 (m, 2H), 1.95-2.05 (m, 2H), 2.68 (bs, 2H), 2.65-2.88 (m, 2H), 3.07 (s, 3H), 6.37 (d, 1H), 7.17-7.26 (m, 1H), 7.68 (dd, 1H), 8.31 (d, 1H), 8.54 (s, 1H), 9.96 (dd, 1H)

### b) (R)-2-(6-Fluoroimidazo[1,2-a]pyridin-3-yl)-N-methyl-N-(piperidin-3-yl)pyrimidin-4-amine

Obtained as a solid (51%) from (R)-tert-butyl 34(2-(6-fluoroimidazo[1,2-a]pyridin-3-yl)pyrimidin-4-yl)(methyl)amino)piperidine-1-carboxylate (Preparation 11a) following the experimental procedure as described in Preparation 5b. The crude product was purified by reverse phase chromatography (C-18 silica from Waters, water/1:1 acetonitrile-methanol as eluents [0.1% v/v formic acid buffered] 0% to 100%).

LRMS (m/z): 327 (M+1)⁺.

¹H-NMR δ (CDCl₃): 1.60-2.02 (m, 6H), 2.58 (td, 1H), 2.79 (t, 1H), 3.04 (s, 3H), 3.13 (t, 1H), 5.30 (s, 1H), 6.33 (d, 1H), 7.22 (ddd, 1H), 7.67 (dd, 1H), 8.27 (d, 1H), 8.54 (s, 1H), 9.98 (ddd, 1H)

### Preparation 12

### N-Ethyl-2-(6-fluoroimidazo[1,2-a]pyridin-3-yl)-N-[(3R)-piperidin-3-yl]pyrimidin-4-amine

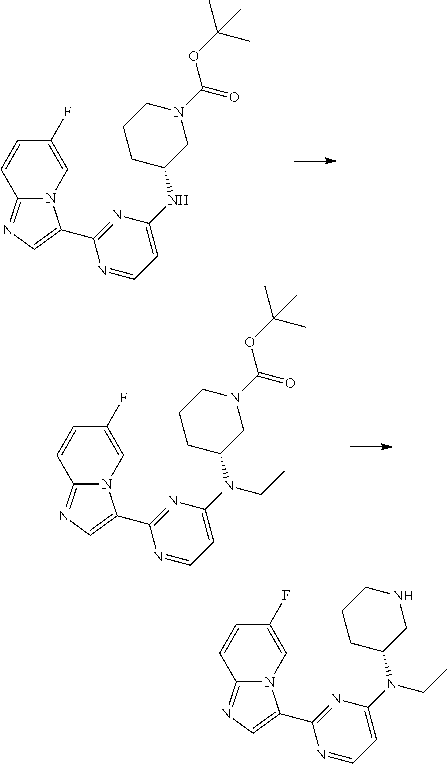 [SMILES: CC(C)(C)OC(=O)N1CCC[C@@H](Nc2ccnc(-c3cnc4ccc(F)cn34)n2)C1 ; CCN(c1ccnc(-c2cnc3ccc(F)cn23)n1)[C@@H]1CCCN(C(=O)OC(C)(C)C)C1 ; CCN(c1ccnc(-c2cnc3ccc(F)cn23)n1)[C@@H]1CCCNC1]

### a) (R)-tert-Butyl-3-(ethyl(2-(6-fluoroimidazo[1,2-a]pyridin-3-yl)pyrimidin-4-yl)amino)piperidine-1-carboxylate

Obtained as a white solid (58%) from tert-butyl (3R)-3-({[2-(6-fluoroimidazo[1,2-a]pyridin-3-yl)pyrimidin-4-yl]oxy}amino)piperidine-1-carboxylate (Preparation 9a) and ethyl iodide following the experimental procedure as described in Preparation 6a. The crude product was purified by flash chromatography (9:1 dichloromethane/methanol).

LRMS (m/z): 441 (M+1)⁺.

### b) N-Ethyl-2-(6-fluoroimidazo[1,2-a]pyridin-3-yl)-N-[(3R)-piperidin-3-yl]pyrimidin-4-amine

Obtained as a solid (100%) from (R)-tert-butyl 3-(ethyl(2-(6-fluoroimidazo[1,2-a]pyridin-3-yl)pyrimidin-4-yl)amino)piperidine-1-carboxylate (Preparation 12a) following the experimental procedure as described in Preparation 5b.

LRMS (m/z): 341 (M+1)⁺.

### Preparation 13

### N-(Cyclopropylmethyl)-2-(6-fluoroimidazo[1,2-a]pyridin-3-yl)-N-[(3R)-piperidin-3-yl]pyrimidin-4-amine

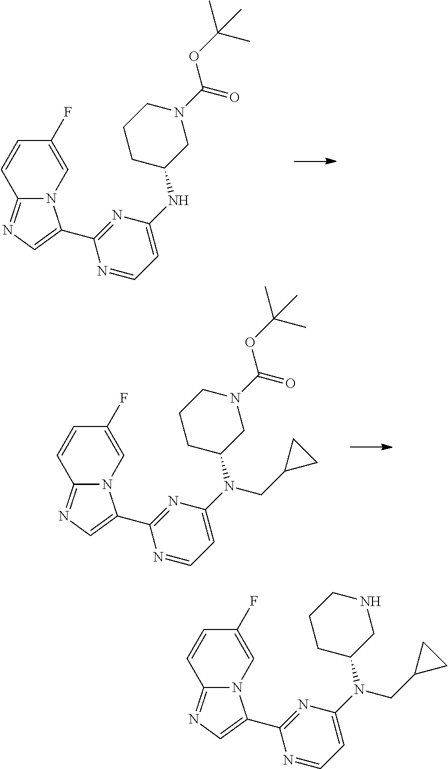 [SMILES: CC(C)(C)OC(=O)N1CCC[C@@H](Nc2ccnc(-c3cnc4ccc(F)cn34)n2)C1 ; CC(C)(C)OC(=O)N1CCC[C@@H](N(CC2CC2)c2ccnc(-c3cnc4ccc(F)cn34)n2)C1 ; Fc1ccc2ncc(-c3nccc(N(CC4CC4)[C@@H]4CCCNC4)n3)n2c1]

### a) tert-Butyl (3R)-3-{(cyclopropylmethyl)[2-(6-fluoroimidazo[1,2-a]pyridin-3-yl)pyrimidin-4-yl]amino}piperidine-1-carboxylate

Obtained as a white solid (47%) from tert-butyl (3R)-3-({[2-(6-fluoroimidazo[1,2-a]pyridin-3-yl)pyrimidin-4-yl]oxy}amino)piperidine-1-carboxylate (Preparation 9a) and (bromomethyl)cyclopropane following the experimental procedure as described in Preparation 6a. The crude product was purified by flash chromatography (92:8 dichloromethane/methanol).

LRMS (m/z): 467 (M+1)⁺.

¹H-NMR δ (CDCl₃): 0.39 (bs, 2H), 0.64 (bs, 2H), 1.12 (bs, 1H), 1.46 (bs, 3H), 1.60 (bs, 6H), 1.87 (bs, 2H), 2.06 (bs, 2H), 2.68 (bs, 1H), 2.88 (bs, 1H), 3.37 (bs, 1H), 3.55 (bs, 2H), 4.02-4.43 (m, 2H), 6.48 (bs, 1H), 7.22 (bs, 1H), 7.68 (bs, 1H), 8.30 (bs, 1H), 8.53 (bs, 1H), 10.01 (bs, 1H)

### b) N-(Cyclopropylmethyl)-2-(6-fluoroimidazo[1,2-a]pyridin-3-yl)-N-[(3R)-piperidin-3-yl]pyrimidin-4-amine

Obtained as a solid (85%) from tert-butyl (3R)-3-{(cyclopropylmethyl)[2-(6-fluoroimidazo[1,2-a]pyridin-3-yl)pyrimidin-4-yl]amino}piperidine-1-carboxylate (Preparation 13a) following the experimental procedure as described in Preparation 5b.

LRMS (m/z): 367 (M+1)⁺.

¹H-NMR δ (CDCl₃): 0.39 (d, 2H), 0.63 (d, 2H), 1.12 (bs, 1H), 1.74-1.94 (m, 2H), 2.03 (bs, 2H), 2.53-2.63 (m, 1H), 2.81 (t, 2H), 3.06-3.25 (m, 3H), 3.39 (bs, 2H), 3.47 (bs, 2H), 6.44 (d, 1H), 7.23 (ddd, 1H), 7.68 (dd, 1H), 8.27 (d, 1H), 8.53 (s, 1H), 10.02 (dd, 1H)

### Preparation 14

### 2-Imidazo[1,2-a]pyridin-3-ylpyrimidin-4-ol

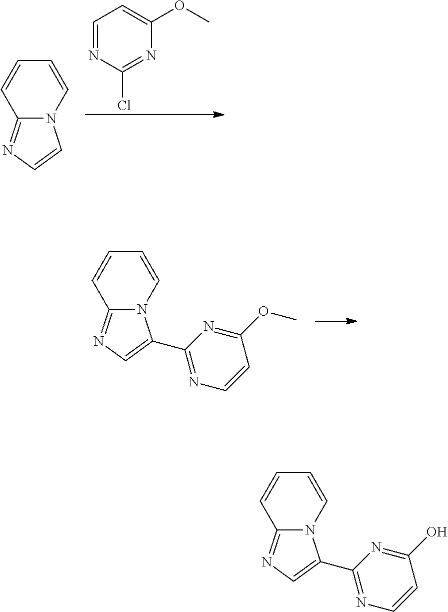

### a) 3-(4-Methoxypyrimidin-2-yl)imidazo[1,2-a]pyridine

Obtained as an off-white solid (39%) from 2-chloro-4-methoxypyrimidine and imidazo[1,2-a]pyridine following the experimental procedure as described in Preparation 4a.

LRMS (m/z): 227 (M+1)⁺.

¹H-NMR δ (DMSO-d₆): 4.12 (s, 3H), 6.84 (d, 1H), 7.26 (t, 1H), 7.57-7.50 (m, 1H), 7.83 (dd, 1H), 8.56 (s, 1H), 8.65 (dd, 1H), 9.93 (dd, 1H).

### b) 2-Imidazo[1,2-a]pyridin-3-ylpyrimidin-4-ol

A stirred mixture of 3-(4-methoxypyrimidin-2-yl)imidazo[1,2-a]pyridine (Preparation 14a, 0.38 g, 1.7 mmol) and aqueous 48% hydrogen bromide (25 mL) was heated at 100° C. After 3 hours, the solvent was evaporated and the residue was treated with isopropyl alcohol. The solid was collected by filtration and dried in vacuum to give the hydrobromide salt of the title compound (0.53 g, 99%) as an off-white solid.

LRMS (m/z): 213 (M+1)⁺.

¹H-NMR δ (DMSO-d₆): 6.63 (d, 1H), 7.66 (t, 1H), 8.13-8.00 (m, 2H), 8.42 (bs, 1H), 8.96 (s, 1H), 10.16 (d, 1H).

### Preparation 15

### 2-Chloro-5-fluoro-4-methoxypyrimidine

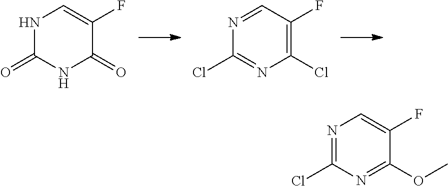

### a) 2,4-Dichloro-5-fluoropyrimidine

To a stirred mixture of 5-fluoropyrimidine-2,4(1H,3H)-dione (3.0 g, 23 mmol) and phosphorous pentachloride (14.41 g, 69 mmol) was added phosphorous oxychloride (12.6 mL, 130 mmol). The reaction mixture was heated to reflux, stirred 5 hours and then cooled to ambient temperature and stirred overnight. The mixture was carefully poured onto ice/water (600 mL) and stirred for 1 hour. Sodium chloride was added and the product was extracted into dichloromethane. The combined organic layer was dried (MgSO₄), filtered and evaporated to give the title compound (84%) as a yellow solid.

LRMS (m/z): 167 (M+1)⁺.

¹H-NMR δ (CDCl₃): 8.49 (s, 1H)

### b) 2-Chloro-5-fluoro-4-methoxypyrimidine

Sodium (0.45 g, 19.6 mmol) was added in portions to methanol (20 mL). Once all the sodium had reacted, a solution of 2,4-dichloro-5-fluoropyrimidine (Preparation 15a, 3.25 g, 19.5 mmol) in methanol (10 mL) was added and the mixture was stirred at ambient temperature overnight. The mixture was diluted with water and extracted with diethyl ether. The organic layer was washed with water, brine, dried (MgSO₄), filtered and the solvent was removed under reduced pressure to give the title compound (81%) as an orange solid, which was used without further purification.

LRMS (m/z): 163 (M+1)⁺.

¹H-NMR δ (CDCl₃): 4.11 (s, 3H), 8.20 (d, 1H)

### Preparation 16

### 5-Fluoro-2-(6-fluoroimidazo[1,2-a]pyridin-3-yl)pyrimidin-4-ol

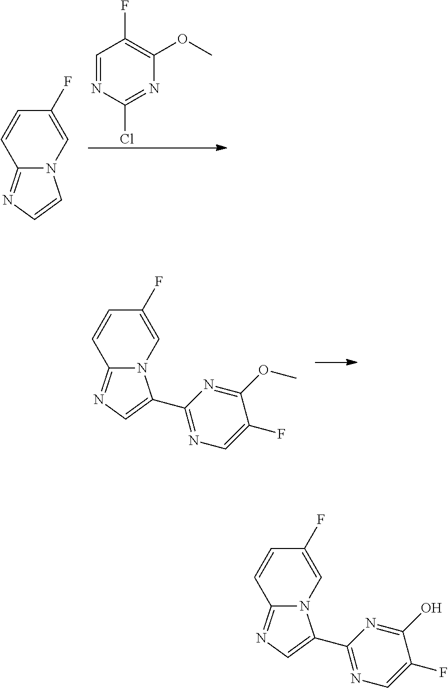

### a) 6-Fluoro-3-(5-fluoro-4-methoxypyrimidin-2-yl)imidazo[1,2-a]pyridine

2-Chloro-5-fluoro-4-methoxypyrimidine (Preparation 15b, 1.10 g, 6.8 mmol) and 6-fluoroimidazo[1,2-a]pyridine (Preparation 2, 1.3 g, 9.5 mmol) were reacted according to the experimental procedure as described in Preparation 4a. The crude product obtained was purified by flash chromatography (10:1 to 1:4 hexanes/ethyl acetate) to give the title compound (10%) as a grey solid.

LRMS (m/z): 263 (M+1)⁺.

¹H-NMR δ (CDCl₃): 4.21 (s, 3H), 7.11 (ddd, 1H), 7.28 (dd, 1H), 8.38 (d, 1H), 8.53 (s, 1H), 9.87 (dd, 1H)

### b) 5-Fluoro-2-(6-fluoroimidazo[1,2-a]pyridin-3-yl)pyrimidin-4-ol

To a solution of 6-fluoro-3-(5-fluoro-4-methoxypyrimidin-2-yl)imidazo[1,2-a]pyridine (Preparation 16a, 200 mg, 0.76 mmol) in ethanol (4 mL) was added a 35% aqueous solution of potassium hydroxide (1.1 mL, 6.9 mmol). The reaction mixture was subjected to microwave irradiation for 1 hour at 130° C. The organic solvent was removed under reduced pressure and the aqueous phase was washed with chloroform then neutralized with 5M hydrochloric acid. The precipitate was filtered and discarded and the filtrate was evaporated to dryness. The residue was triturated with several portions of a chloroform/methanol mixture and then dried in vacuum to give the title compound (59%) as a grey solid, which was used without further purification.

LRMS (m/z): 249 (M+1)⁺.

### Preparation 17

### (R)-5-Fluoro-2-(6-fluoroimidazo[1,2-a]pyridin-3-yl)-N-(piperidin-3-yl)pyrimidin-4-amine

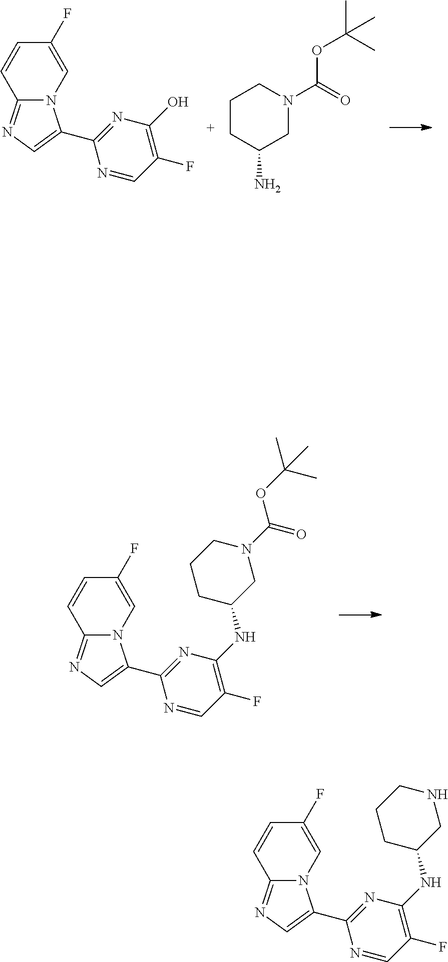 [SMILES: Oc1nc(-c2cnc3ccc(F)cn23)ncc1F ; CC(C)(C)OC(=O)N1CCC[C@@H](N)C1 ; CC(C)(C)OC(=O)N1CCC[C@@H](Nc2nc(-c3cnc4ccc(F)cn34)ncc2F)C1 ; Fc1ccc2ncc(-c3ncc(F)c(N[C@@H]4CCCNC4)n3)n2c1]

### a) (R)-tert-Butyl 3-(5-fluoro-2-(6-fluoroimidazo[1,2-a]pyridin-3-yl)pyrimidin-4-ylamino)piperidine-1-carboxylate

Obtained as a solid (22%) from 5-fluoro-2-(6-fluoroimidazo[1,2-a]pyridin-3-yl)pyrimidin-4-ol (Preparation 16b) and (R)-tert-butyl 3-aminopiperidine-1-carboxylate following the experimental procedure as described in Preparation 5a. The crude product was purified by flash chromatography (9:1 dichloromethane/methanol).

LRMS (m/z): 431 (M+1)⁺.

### b) (R)-5-Fluoro-2-(6-fluoroimidazo[1,2-a]pyridin-3-yl)-N-(piperidin-3-yl)pyrimidin-4-amine

Obtained as a solid (100%) from (R)-tert-butyl 3-(5-fluoro-2-(6-fluoroimidazo[1,2-a]pyridin-3-yl)pyrimidin-4-ylamino)piperidine-1-carboxylate (Preparation 17a) following the experimental procedure as described in Preparation 5b.

LRMS (m/z): 331 (M+1)⁺.

### Preparation 18

### 2-Chloro-4-methoxy-5-methylpyrimidine

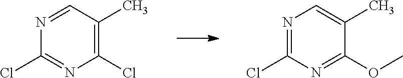

Obtained as a white solid (100%) from 2,4-dichloro-5-methylpyrimidine and sodium methoxide following the experimental procedure as described in Preparation 15b.

LRMS (m/z): 159 (M+1)⁺.

¹H-NMR δ (CDCl₃): 2.12 (d, 3H), 4.03 (s, 3H), 8.10 (d, 1H)

### Preparation 19

### 2-(6-Fluoroimidazo[1,2-a]pyridin-3-yl)-5-methylpyrimidin-4-ol

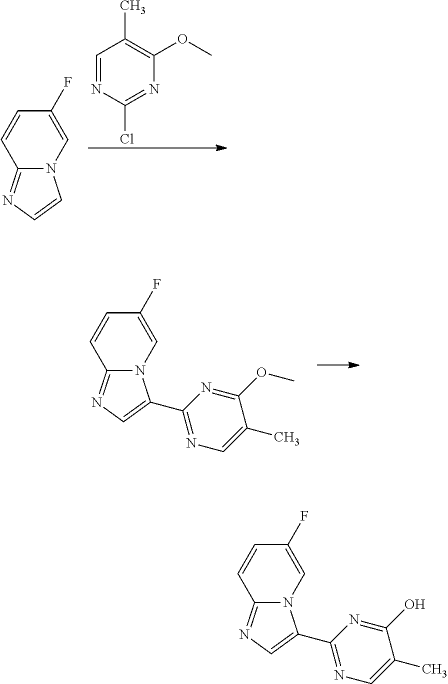

### a) 6-Fluoro-3-(4-methoxy-5-methylpyrimidin-2-yl)imidazo[1,2-a]pyridine

6-Fluoroimidazo[1,2-a]pyridine (Preparation 2, 1.0 g, 7.3 mmol) and 2-chloro-4-methoxy-5-methylpyrimidine (Preparation 18, 1.70 g, 10.7 mmol) were reacted according to the experimental procedure as described in Preparation 4a. The crude material obtained was purified by reverse phase chromatography (C-18 silica from Waters, water/1:1 acetonitrile-methanol as eluents [0.1% v/v formic acid buffered] 0% to 100%) to give the title compound (24%) as a white solid.

LRMS (m/z): 259 (M+1)⁺.

¹H-NMR δ (CDCl₃): 2.18 (s, 3H), 4.12 (s, 3H), 7.24 (dd, 1H), 7.64-7.72 (m, 1H), 8.29 (d, 1H), 8.55 (s, 1H), 9.95 (dt, 1H)

### b) 2-(6-Fluoroimidazo[1,2-a]pyridin-3-yl)-5-methylpyrimidin-4-ol

6-Fluoro-3-(4-methoxy-5-methylpyrimidin-2-yl)imidazo[1,2-a]pyridine (Preparation 19a, 0.45 g, 1.7 mmol) and a 35% aqueous solution of potassium hydroxide (2.79 mL, 17.4 mmol) were reacted according to the experimental procedure as described in Preparation 16b. The reaction was concentrated in vacuum and water was added. The solution was neutralized with 2M aqueous hydrochloric acid and the resultant precipitate was filtered and dried to give the title compound (100%) as a white solid.

LRMS (m/z): 243 (M−1)⁺.

¹H NMR □ (DMSO-d₆): 3.33 (s, 3H), 7.60-7.66 (m, 1H), 7.87 (dd, 1H), 8.03 (brs, 1H), 8.72 (s, 1H), 9.91 (bs, 1H).

### Preparation 20

### 2-(6-Fluoroimidazo[1,2-a]pyridin-3-yl)-5-methyl-N-[(3R)-piperidin-3-yl]pyrimidin-4-amine

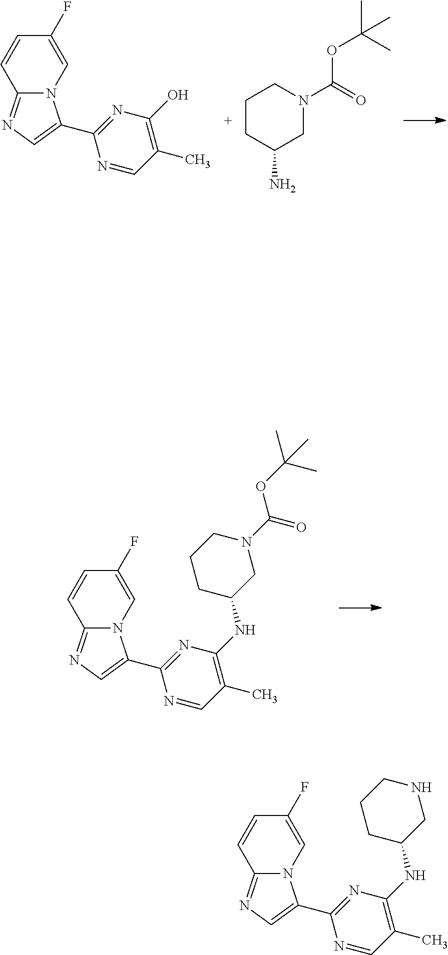 [SMILES: Cc1cnc(-c2cnc3ccc(F)cn23)nc1O ; CC(C)(C)OC(=O)N1CCC[C@@H](N)C1 ; Cc1cnc(-c2cnc3ccc(F)cn23)nc1N[C@@H]1CCCN(C(=O)OC(C)(C)C)C1 ; Cc1cnc(-c2cnc3ccc(F)cn23)nc1N[C@@H]1CCCNC1]

### a) tert-Butyl (3R)-3-{[2-(6-fluoroimidazo[1,2-a]pyridin-3-yl)-5-methylpyrimidin-4-yl]amino}piperidine-1-carboxylate

Obtained as a pale yellow solid (35%) from 2-(6-fluoroimidazo[1,2-a]pyridin-3-yl)-5-methylpyrimidin-4-ol (Preparation 19b) and (R)-tert-butyl 3-aminopiperidine-1-carboxylate following the experimental procedure as described in Preparation 5a. The crude product was purified by flash chromatography (9:1 dichloromethane/methanol).

LRMS (m/z): 427 (M+1)⁺.

¹H-NMR δ (CDCl₃): 1.51 (bs, 2H), 1.72 (bs, 9H), 2.01-2.29 (m, 5H), 3.42 (bs, 1H), 3.56-3.83 (m, 3H), 4.37 (bs, 1H), 7.27 (bs, 1H), 7.73 (bs, 1H), 8.13 (bs, 1H), 8.57 (bs, 1H), 10.02 (bs, 1H)

### b) 2-(6-Fluoroimidazo[1,2-a]pyridin-3-yl)-5-methyl-N-[(3R)-piperidin-3-yl]pyrimidin-4-amine

Obtained as a white solid (70%) from tert-butyl (3R)-3-{[2-(6-fluoroimidazo[1,2-a]pyridin-3-yl)-5-methylpyrimidin-4-yl]amino}piperidine-1-carboxylate (Preparation 20a) following the experimental procedure as described in Preparation 5b.

LRMS (m/z): 327 (M+1)⁺.

¹H-NMR δ (CDCl₃): 1.69 (bs, 3H), 1.74-2.04 (m, 4H), 2.87 (bs, 1H), 3.21 (bs, 3H), 4.35 (bs, 1H), 5.23 (bs, 1H), 7.21 (bs, 1H), 7.66 (bs, 1H), 8.05 (bs, 1H), 8.50 (s, 1H), 9.99 (bs, 1H)

### Preparation 21

### 2-(6-Fluoroimidazo[1,2-a]pyridin-3-yl)-N-pyrrolidin-3-ylpyrimidin-4-amine

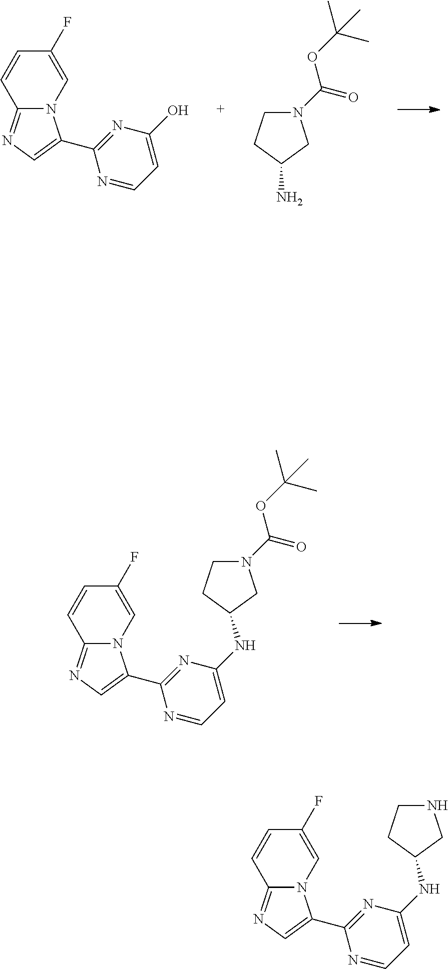 [SMILES: Oc1ccnc(-c2cnc3ccc(F)cn23)n1 ; CC(C)(C)OC(=O)N1CC[C@@H](N)C1 ; CC(C)(C)OC(=O)N1CC[C@@H](Nc2ccnc(-c3cnc4ccc(F)cn34)n2)C1 ; Fc1ccc2ncc(-c3nccc(N[C@@H]4CCNC4)n3)n2c1]

### a) tert-Butyl 3-{[2-(6-fluoroimidazo[1,2-a]pyridin-3-yl)pyrimidin-4-yl]amino}pyrrolidine-1-carboxylate

Obtained as an oil (44%) from 2-(6-fluoroimidazo[1,2-a]pyridin-3-yl)pyrimidin-4-ol (Preparation 8b) and (R)-tert-butyl 3-aminopyrrolidine-1-carboxylate following the experimental procedure as described in Preparation 5a. The crude product was purified by reverse phase chromatography (C-18 silica from Waters, water/1:1 acetonitrile-methanol as eluents [0.1% v/v formic acid buffered] 0% to 100%).

LRMS (m/z): 399 (M+1)⁺.

### b) 2-(6-Fluoroimidazo[1,2-a]pyridin-3-yl)-N-pyrrolidin-3-ylpyrimidin-4-amine

Obtained as an oil (77%) from tert-butyl 3-{[2-(6-fluoroimidazo[1,2-a]pyridin-3-yl)pyrimidin-4-yl]amino}pyrrolidine-1-carboxylate (Preparation 21a) following the experimental procedure as described in Preparation 5b.

LRMS (m/z): 299 (M+1)⁺.

¹H-NMR δ (CDCl₃): 0.83-0.98 (m, 1H), 1.82 (ddd, 1H), 2.30 (dd, 1H), 2.97-3.11 (m, 2H), 3.15-3.24 (m, 1H), 3.25-3.34 (m, 1H), 5.31 (d, 1H), 6.21 (d, 1H), 7.24 (dd, 1H), 7.68 (dd, 1H), 8.26 (d, 1H), 8.53 (s, 1H), 10.00 (dd, 1H)

### Preparation 22

### 3-[4-(1,4-Diazepan-1-yl)pyrimidin-2-yl]imidazo[1,2-a]pyridine-6-carbonitrile

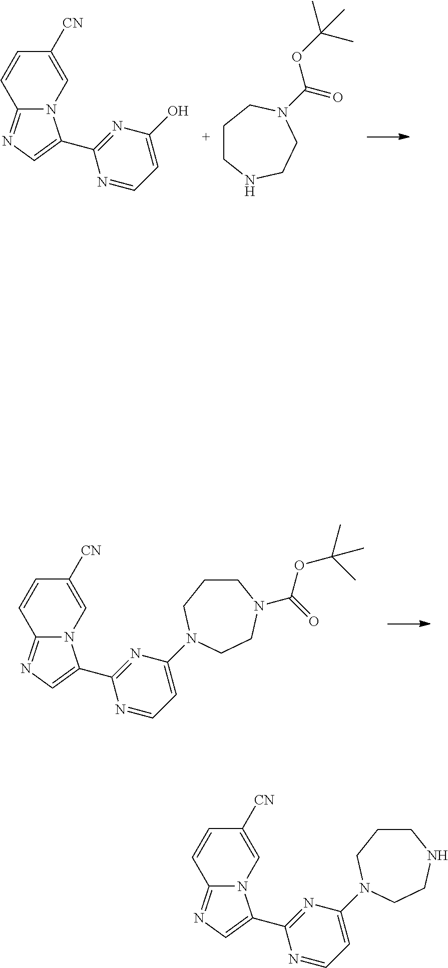 [SMILES: N#Cc1ccc2ncc(-c3nccc(O)n3)n2c1 ; CC(C)(C)OC(=O)N1CCCNCC1 ; CC(C)(C)OC(=O)N1CCCN(c2ccnc(-c3cnc4ccc(C#N)cn34)n2)CC1 ; N#Cc1ccc2ncc(-c3nccc(N4CCCNCC4)n3)n2c1]

### a) tert-Butyl 4-[2-(6-cyanoimidazo[1,2-a]pyridin-3-yl)pyrimidin-4-yl]-1,4-diazepane-1-carboxylate

Obtained as a solid (48%) from 3-(4-hydroxypyrimidin-2-yl)imidazo[1,2-a]pyridine-6-carbonitrile (Preparation 4b) and tert-butyl 1,4-diazepane-1-carboxylate following the experimental procedure as described in Preparation 5a. The crude product was used without further purification.

LRMS (m/z): 420 (M+1)⁺.

¹H-NMR δ (DMSO-d₆): 1.03 (bs, 3H), 1.26 (bs, 4H), 1.56-1.98 (m, 4H), 3.20-3.33 (m, 2H), 3.51-3.78 (m, 4H), 3.81-4.05 (m, 2H), 6.71 (d, 1H), 7.70 (d, 1H), 7.91 (d, 1H), 8.32 (bs, 1H), 8.51 (s, 1H), 10.42 (bs, 1H)

### b) 3-[4-(1,4-Diazepan-1-yl)pyrimidin-2-yl]imidazo[1,2-a]pyridine-6-carbonitrile

Obtained as a solid (65%) from tert-butyl 4-(2-(6-cyanoimidazo[1,2-a]pyridin-3-yl)pyrimidin-4-yl)-1,4-diazepane-1-carboxylate (Preparation 22a) following the experimental procedure as described in Preparation 5b.

LRMS (m/z): 320 (M+1)⁺.

### Preparation 23

### 2-Chloro-4-methoxyquinazoline

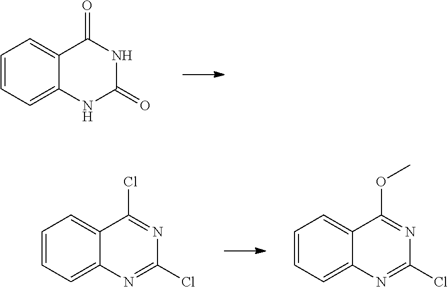

### a) 2,4-Dichloroquinazoline

Quinazoline-2,4(1H,3H)-dione (5.0 g, 30.8 mmol) was dissolved in phosphorous oxychloride (50 mL, 546 mmol) and the mixture was stirred and heated to 115° C. After 16 hours, the mixture was evaporated and then co-evaporated with toluene. The residue was dissolved in ethyl acetate and triethylamine (20 mL) was added. After stirring for 5 minutes, water was added. The organic phase was washed with water, brine, dried (MgSO₄) and evaporated to give the title compound (6.45 g, 100%) as an off-white solid.

LRMS (m/z): 199 (M+1)⁺.

¹H-NMR δ (CDCl₃): 7.76 (ddd, 1H), 8.03-7.99 (m, 2H), 8.28 (dt, 1H).

### b) 2-Chloro-4-methoxyquinazoline

To a stirred solution of 2,4-dichloroquinazoline (Preparation 23a, 2.0 g, 10.0 mmol) in methanol (100 mL) was added a 5M solution of sodium methoxide in methanol (2.1 mL, 10.5 mmol) and the mixture was stirred at ambient temperature for 48 hours. The solvent was evaporated and the mixture was partitioned between ethyl acetate and water. The organic phase was dried (MgSO₄) and evaporated to give the title compound (1.87 g, 96%) as a solid.

LRMS (m/z): 195 (M+W.

¹H-NMR δ (CDCl₃): 4.23 (s, 3H), 7.57 (ddd, 1H), 7.88-7.84 (m, 2H), 8.17-8.11 (m, 1H).

### Preparation 24

### (2-(4-Hydroxyquinazolin-2-yl)imidazo[1,2-a]pyridine-6-carbonitrile

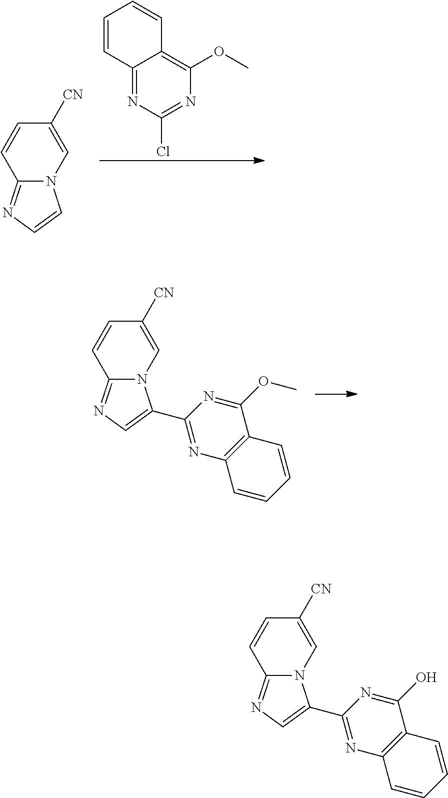 [SMILES: N#Cc1ccc2nccn2c1 ; COc1nc(Cl)nc2ccccc12 ; COc1nc(-c2cnc3ccc(C#N)cn23)nc2ccccc12 ; N#Cc1ccc2ncc(-c3nc(O)c4ccccc4n3)n2c1]

### a) 2-(4-Methoxyquinazolin-2-yl)imidazo[1,2-a]pyridine-6-carbonitrile

Obtained as a white solid (74%) from imidazo[1,2-a]pyridine-6-carbonitrile (Preparation 1, 0.15 g, 1.1 mmol) and 2-chloro-4-methoxyquinazoline (Preparation 23b, 0.32 g, 1.6 mmol) following the experimental procedure as described in Preparation 4a.

LRMS (m/z): 302 (M+1)⁺.

¹H-NMR δ (CDCl₃): 4.30 (s, 3H), 7.63-7.38 (m, 2H), 7.86 (dd, 1H), 8.02 (d, 1H), 8.18 (d, 1H), 8.76 (s, 1H), 10.83 (s, 1H).

### b) (2-(4-Hydroxyquinazolin-2-yl)imidazo[1,2-a]pyridine-6-carbonitrile

Obtained as a brown solid (74%) from 3-(4-methoxyquinazolin-2-yl)imidazo[1,2-a]pyridine-6-carbonitrile (Preparation 24a, 0.30 g, 1.0 mmol) following the experimental procedure as described in Preparation 4b.

LRMS (m/z): 286 (M+1)⁺.

### Preparation 25

### 2-Chloro-4-methoxy-5-pyridin-3-ylpyrimidine

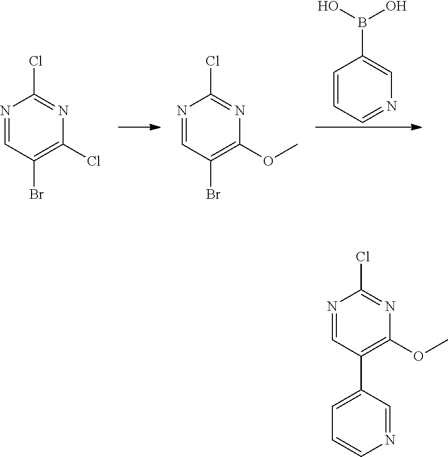

### a) 5-Bromo-2-chloro-4-methoxypyrimidine

A 30% solution of sodium methoxide in methanol (8.0 mL, 42.0 mmol) was added dropwise to a cooled (ice-bath) solution of 5-bromo-2,4-dichloropyrimidine (9.85 g, 43.2 mmol) in methanol (100 mL). The mixture was warmed to room temperature and stirred for 4 hours. The solvent was removed under reduced pressure and the residue was partitioned between water and dichloromethane. The organic layer was washed with water, brine, dried (MgSO₄) and evaporated to give the title compound (9.40 g, 93%) as a solid.

LRMS (m/z): 222/224 (M+1)⁺.

¹H-NMR δ (CDCl₃): 4.11 (s, 3H), 8.44 (s, 1H).

### b) 2-Chloro-4-methoxy-5-pyridin-3-ylpyrimidine

Nitrogen was bubbled for 5 minutes through a mixture of 5-bromo-2-chloro-4-methoxypyrimidine (Preparation 25a, 0.51 g, 2.3 mmol), pyridin-3-yl boronic acid (0.30 g, 2.5 mmol) and potassium carbonate (0.93 g, 6.7 mmol) in toluene (8.0 mL) and N,N′-dimethylformamide (1.0 mL) contained in a microwave vessel. Then [1,1′-bis(diphenylphosphino)ferrocene] dichloropalladium (II) complex with dichloromethane (1:1) (68 mg, 0.11 mmol) was added and the vessel was sealed and subjected to microwave irradiation for 10 minutes at 185° C. The reaction mixture was filtered through Celite®, washing the filter cake with ethyl acetate. The combined filtrate and washings were evaporated and the residue was purified by flash chromatography (2:1 hexanes/ethyl acetate) to give the title compound (0.066 g, 14%) as a solid.

LRMS (m/z): 222 (M+1)⁺

### Preparation 26

### 3-(4-Hydroxy-5-pyridin-3-ylpyrimidin-2-yl)imidazo[1,2-a]pyridine-6-carbonitrile

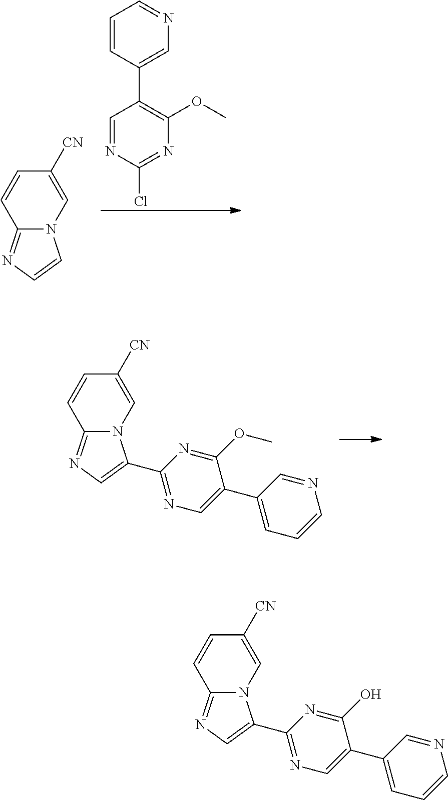

### a) 3-(4-Methoxy-5-pyridin-3-ylpyrimidin-2-yl)imidazo[1,2-a]pyridine-6-carbonitrile

2-Chloro-4-methoxy-5-pyridin-3-yl-pyrimidine (Preparation 25b, 0.226 g, 1.0 mmol) and imidazo[1,2-a]pyridine-6-carbonitrile (Preparation 1, 0.217 g, 1.5 mmol) were reacted according to the experimental procedure as described in Preparation 4a. The crude product was purified by flash chromatography (9:1 ethyl acetate/methanol) to give the title compound (14%) as an off-white solid.

LRMS (m/z): 329 (M+1)⁺

¹H-NMR δ (CDCl₃): 4.19 (s, 3H), 6.50 (dd, 1H), 7.44 (ddd, 1H), 7.48 (dd, 1H), 7.85 (dd, 1H), 7.96 (ddd, 1H), 8.62 (s, 1H), 8.67 (dd, 1H), 8.87 (dd, 1H), 10.56 (dd, 1H).

### b) 3-(4-Hydroxy-5-pyridin-3-ylpyrimidin-2-yl)imidazo[1,2-a]pyridine-6-carbonitrile

Obtained as a brown solid (100%) from 3-(4-methoxy-5-pyridin-3-ylpyrimidin-2-yl)imidazo[1,2-a]pyridine-6-carbonitrile (Preparation 26a) following the experimental procedure as described in Preparation 4b.

LRMS (m/z): 315 (M+1)⁺.

¹H-NMR δ (CDCl₃): 6.86 (d, 1H), 7.91 (d, 1H), 8.04 (d, 1H), 8.19-8.10 (m, 1H), 8.81 (s, 1H), 9.05-8.86 (m, 2H), 9.40 (s, 1H), 10.43 (s, 1H).

### Preparation 27

### 3-(4-Chloro-6-pyridin-3-ylpyrimidin-2-yl)imidazo[1,2-a]pyridine

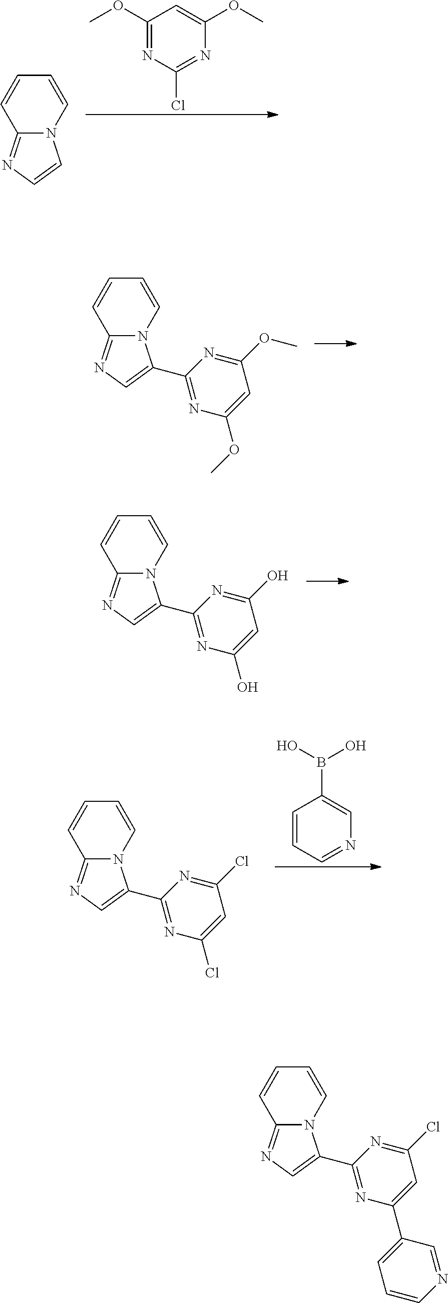

### a) 3-(4,6-Dimethoxypyrimidin-2-yl)imidazo[1,2-a]pyridine

Obtained as a brown solid (50%) from 2-chloro-4,6-dimethoxypyrimidine and imidazo[1,2-a]pyridine following the experimental procedure as described in Preparation 4a.

LRMS (m/z): 282 (M+1)⁺.

¹H-NMR δ (CDCl₃): 4.03 (s, 6H), 6.12 (s, 1H), 7.23 (td, 1H), 7.50 (ddd, 1H), 7.78 (dt, 1H), 8.52 (s, 1H), 9.83 (dt, 1H).

### b) 2-Imidazo[1,2-a]pyridin-3-ylpyrimidine-4,6-diol

Obtained as a brown solid (100%) from 3-(4,6-dimethoxypyrimidin-2-yl)imidazo[1,2-a]pyridine (Preparation 27a) following the experimental procedure as described in Preparation 4b.

¹H-NMR δ (DMSO-d₆): 5.72 (s, 1H), 7.69 (ddd, 1H), 8.14-8.00 (m, 2H), 8.89 (s, 1H), 10.30 (d, 1H).

### c) 3-(4,6-Dichloropyrimidin-2-yl)imidazo[1,2-a]pyridine

A solution of 2-imidazo[1,2-a]pyridin-3-ylpyrimidine-4,6-diol (Preparation 27b, 330 mg, 1.45 mmol) in phosphorous (V) oxychloride (2.8 mL) was heated and stirred to 115° C. After 18 hours, the mixture was evaporated and the residue was co-evaporated with toluene to give the title compound (154 mg, 40%).

LRMS (m/z): 291 (M+1)⁺.

¹H-NMR δ (CDCl₃): 7.48 (s, 1H), 7.75 (t, 1H), 8.20 (t, 1H), 8.48 (d, 1H), 8.90 (s, 1H), 10.21 (d, 1H).

### d) 3-(4-Chloro-6-pyridin-3-ylpyrimidin-2-yl)imidazo[1,2-a]pyridine

Nitrogen was bubbled for 5 minutes through a mixture of 3-(4,6-dichloropyrimidin-2-yl)imidazo[1,2-a]pyridine (Preparation 27c, 0.14 g, 0.54 mmol), pyridin-3-yl boronic acid (0.044 g, 0.36 mmol) and 2M aqueous cesium carbonate solution (0.54 mL, 1.08 mmol) in 1,4-dioxane (6 mL) contained in a Schlenck tube. Then [1,1′-bis(diphenylphosphino) ferrocene] dichloropalladium (II) complex with dichloromethane (1:1) (0.029 g, 0.04 mmol) was added and the vessel was sealed and the contents were stirred and heated to 90° C. After 18 hours, the reaction mixture was filtered through Celite®, washing the filter cake with ethyl acetate. The filtrate was evaporated and the residue was purified by flash chromatography (20:1 dichloromethane/ethanol) to give the title compound (0.038 g, 32%) as a solid.

LRMS (m/z): 333 (M+1)⁺.

### Preparation 28

### 2-Chloro-6-[(4-methoxybenzyl)oxy]pyrazine

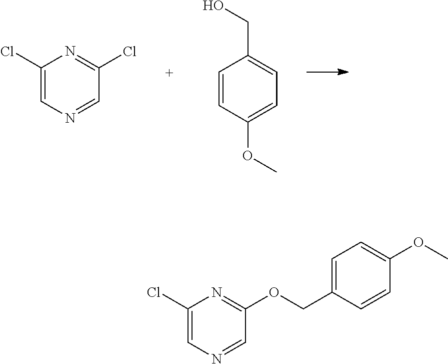

Sodium hydride (60% dispersion in mineral oil, 0.16 g, 4.0 mmol) was added portionwise to a stirred solution of 2,6-dichloropyrazine (0.50 g, 3.4 mmol) and (4-methoxyphenyl)methanol (0.51 g, 3.7 mmol) in N,N′-dimethylformamide. The reaction mixture was stirred at ambient temperature overnight then evaporated in vacuum. The residue was partitioned between ethyl acetate and water and the organic layer was washed with brine, dried (MgSO₄) and evaporated. The residue was purified by flash chromatography (9:1 to 4:1 hexanes/ethyl acetate) to give the title compound (0.687 g, 81%) as a solid.

LRMS (m/z): 251 (M+1)⁺.

¹H-NMR δ (CDCl₃): 3.83 (s, 3H), 5.32 (s, 2H), 6.93 (d, 2H), 7.40 (d, 2H), 8.14 (s, 1H), 8.15 (s, 1H)

### Preparation 29

### 3-(6-Hydroxypyrazin-2-yl)imidazo[1,2-a]pyridine-6-carbonitrile

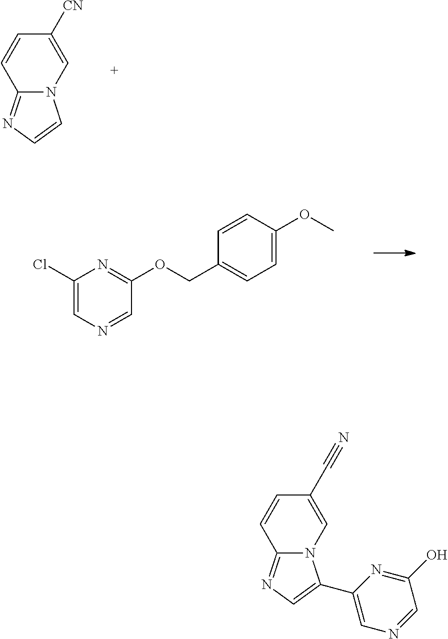 [SMILES: N#Cc1ccc2nccn2c1 ; COc1ccc(COc2cncc(Cl)n2)cc1 ; N#Cc1ccc2ncc(-c3cncc(O)n3)n2c1]

An oven dried resealable Schlenk tube was charged with 2-chloro-6-(4-methoxybenzyloxy)pyrazine (Preparation 28, 0.68 g, 2.7 mmol), imidazo[1,2-a]pyridine-6-carbonitrile (Preparation 1, 0.59 g, 4.1 mmol), potassium acetate (0.138 g, 1.41 mmol) and N,N′-dimethylacetamide (4 mL). The Schlenk tube was subjected to three cycles of evacuation-backfilling with argon then tetrakis(triphenylphospino)palladium(0) (0.34 g, 0.3 mol) was added. After three further cycles of evacuation-backfilling with argon, the Schlenk tube was capped and placed in an oil bath at 150° C. and the mixture was stirred for 4 hours. The mixture was evaporated in vacuum and the residue was partitioned between ethyl acetate and water. The organic layer was extracted with 2M aqueous hydrochloric acid and the aqueous layer was taken to pH 5-6 with solid sodium hydrogen carbonate and then extracted with ethyl acetate. The organic layer was dried (MgSO₄), evaporated and the residue was purified by column chromatography (98:2 to 90:10 dichloromethane/methanol) to give the title compound (0.102 g, 10%).

LRMS (m/z): 238 (M+1)⁺.

### Preparation 30

### 2-Chloro-N-(4-methoxybenzyl)-N-[(1S)-1-phenylethyl]pyrimidin-4-amine

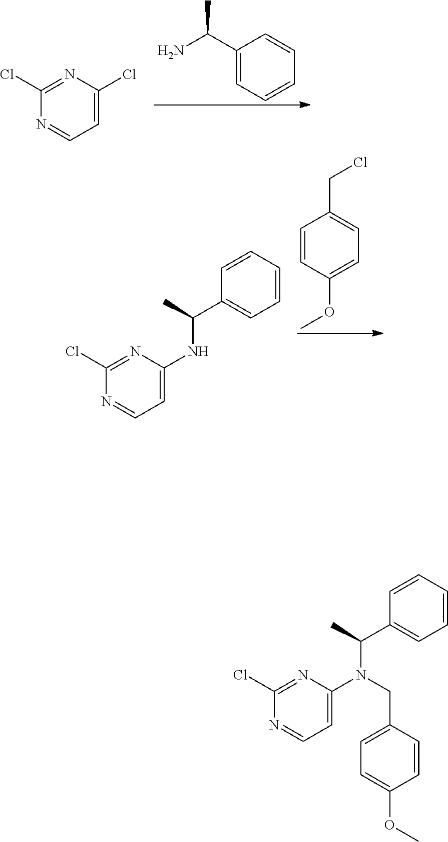

### a) 2-Chloro-N-[(1S)-1-phenylethyl]pyrimidin-4-amine

A stirred mixture of 2,4-dichloropyrimidine (1.27 g, 8.5 mmol), (S)-1-phenylethanamine (1.0 mL, 7.8 mmol) and diisopropylethylamine (1.6 mL, 8.6 mmol) in N,N′-dimethylformamide (12 mL) was heated to 90° C. in a sealed tube. After 16 hours, the mixture was evaporated in vacuum and then taken up in ethyl acetate. The organic layer was washed with water and brine, dried (MgSO₄) and evaporated. Purification by flash chromatography (300:1 dichloromethane/methanol) gave the title compound (0.65 g, 36%) as a pale yellow oil.

LRMS (m/z): 234 (M+1)⁺.

¹H-NMR δ (CDCl₃): 1.59 (d, 3H), 5.38-5.75 (m, 1H), 6.08 (d, 1H), 7.28-7.43 (m, 5H), 7.97 (d, 1H)

### b) 2-Chloro-N-(4-methoxybenzyl)-N-[(1S)-1-phenylethyl]pyrimidin-4-amine

Sodium hydride (60% dispersion in mineral oil, 0.17 g, 4.3 mmol) was added portionwise to a solution of 2-chloro-N-[(1S)-1-phenylethyl]pyrimidin-4-amine (Preparation 30a, 0.95 g, 4.1 mmol) in N,N′-dimethylformamide (10 mL). The mixture was stirred for 15 minutes, then 1-(chloromethyl)-4-methoxybenzene (0.58 mL, 4.3 mmol) was added and the reaction was stirred at room temperature for a further 5 hours. Water was added and the mixture was extracted with ethyl acetate. The organic layer was washed with water, brine, dried (MgSO₄) and evaporated in vacuum. The residue was triturated with several portions of hexane to give a thick oil. Purification by flash chromatography (5:1 hexanes/ethyl acetate) gave the title compound (0.94 g, 65%) as a white solid.

LRMS (m/z): 354 (M+1)⁺.

¹H-NMR δ (CDCl₃): 1.58 (d, 3H), 3.78 (bs, 3H), 4.21-4.44 (m, 3H), 6.11 (d, 1H), 6.80 (d, 2H), 6.99 (d, 2H), 7.15-7.42 (m, 5H), 7.92 (d, 1H)

### Preparation 31

### N-Benzyl-2-chloro-N-methylpyrimidin-4-amine

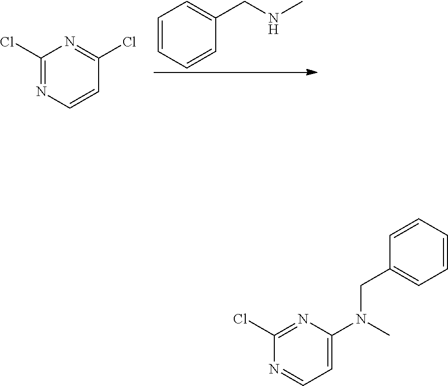

N-Methyl-1-phenylmethanamine (0.32 mL, 2.5 mmol) and diisopropylethylamine amine (0.58 mL, 3.3 mmol) were added sequentially to a solution of 2,4-dichloropyrimidine (0.50 g, 3.4 mmol) in N,N′-dimethylformamide (5 mL). The mixture was stirred in a sealed tube for 3 hours at 90° C. The mixture was evaporated and the residue was dissolved in ethyl acetate and washed with water, dried (MgSO₄), filtered and evaporated. Purification by flash chromatography (3:1 hexanes/ethyl acetate) gave the title compound (0.51 g, 86%) as an oil.

LRMS (m/z): 234 (M+1)⁺.

¹H-NMR δ (CDCl₃): 3.08 (bs, 3H), 4.79 (bs, 2H), 6.35 (d, 1H), 7.14-7.41 (m, 5H), 8.04 (d, 1H)

### Preparation 32

### (S)-6-Chloro-N-(1-phenylethyl)pyrazin-2-amine

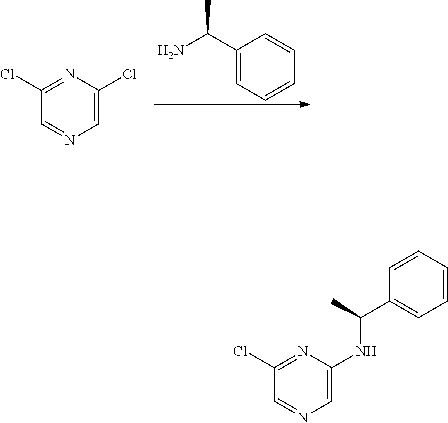

Obtained as a white solid (37%) from 2,6-dichloropyrazine (1.92 g, 12.9 mmol) and (S)-1-phenylethanamine (1.72 g, 14.2 mmol) following the experimental procedure as described in WO200399796.

LRMS (m/z): 234 (M+1)⁺.

¹H-NMR δ (CDCl₃): 1.58 (d, 3H), 4.86 (bs, 1H), 5.08 (bs, 1H), 7.24-7.32 (m, 5H), 7.60 (s, 1H), 7.79 (s, 1H)

### Preparation 33

### 1-(Ethylsulfonyl)-N,4-dimethylpiperidin-3-amine

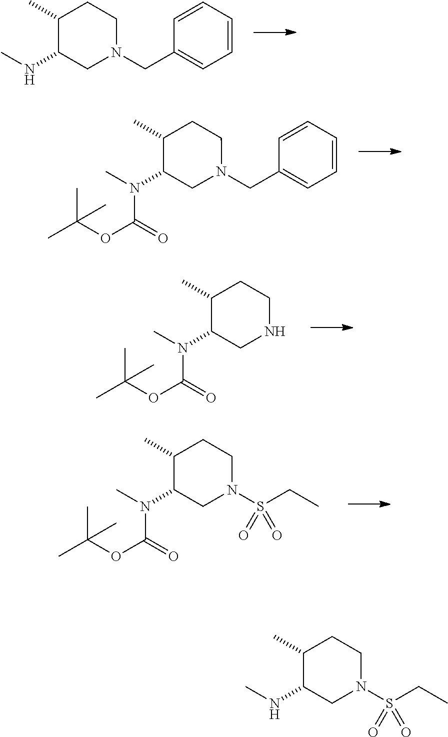

### a) tert-Butyl (1-benzyl-4-methylpiperidin-3-yl)methylcarbamate

Triethylamine (1.72 mL, 12.3 mmol) and di-tert-butyl dicarbonate (2.70 g, 12.4 mmol) were added sequentially to a stirred solution of cis-1-benzyl-N,4-dimethylpiperidin-3-amine (prepared as described in WO200560972; 2.70 g, 12.4 mmol) in dichloromethane (60 mL). The mixture was stirred at ambient temperature overnight. The mixture was washed with saturated aqueous sodium hydrogencarbonate solution, water, brine, dried (MgSO₄) and the solvent was removed under reduced pressure to give the title compound (3.40 g, 86%) as a solid.

LRMS (m/z): 319 (M+1)⁺.

¹H-NMR δ (CDCl₃): 1.00 (d, 3H), 1.51 (s, 9H), 1.64 (bs, 1H), 1.98 (bs, 2H), 2.24 (bs, 2H), 2.49 (dd, 1H), 2.77 (bs, 2H), 3.16 (s, 3H), 3.50 (s, 2H), 7.23-7.45 (m, 5H)

### b) tert-Butyl methyl(4-methylpiperidin-3-yl)carbamate

Palladium on charcoal (10%, 0.30 g) was added to a solution of tert-butyl (1-benzyl-4-methylpiperidin-3-yl)methylcarbamate (Preparation 33a, 3.40 g, 10.7 mmol) in methanol (70 mL) and the mixture was stirred under an atmosphere of hydrogen for 20 hours. The catalyst was filtered off and the solvent was removed under reduced pressure to give the title compound (2.38 g, 97%) as an oil.

LRMS (m/z): 229 (M+1)⁺.

1H-NMR δ (CDCl₃): 0.94 (d, 3H), 1.39 (s, 9H), 1.58-1.68 (m, 1H), 2.11-2.20 (m, 2H), 2.68 (bs, 1H), 2.73-2.81 (m, 2H), 2.83 (s, 3H), 2.99 (bs, 2H)

### c) tert-Butyl [1-(ethylsulfonyl)-4-methylpiperidin-3-yl]methylcarbamate

A solution of ethanesulfonyl chloride (0.86 mL, 9.1 mmol) in dichloromethane (3 mL) was added dropwise to a stirred, cooled (ice-bath) solution of tert-butyl methyl(4-methylpiperidin-3-yl)carbamate (Preparation 33b, 1.89 g, 8.3 mmol) in dichloromethane (60 mL) and triethylamine (1.27 mL, 9.1 mmol). The mixture was warmed to room temperature and stirred overnight. The mixture was washed with saturated aqueous sodium hydrogencarbonate solution, water, brine, dried (MgSO₄) and the solvent was removed under reduced pressure to give the title compound (2.20 g, 83%) as a solid.

LRMS (m/z): 321 (M+1)⁺.

¹H-NMR δ (CDCl₃): 1.00 (d, 3H), 1.38 (t, 3H), 1.46 (s, 9H), 1.65-1.76 (m, 2H), 2.04-2.14 (m, 1H), 2.96 (d, 2H), 2.98 (s, 3H), 3.05-3.18 (m, 2H), 3.33 (bs, 1H), 3.51 (bs, 1H), 3.64 (bs, 1H)

### d) 1-(Ethylsulfonyl)-N,4-dimethylpiperidin-3-amine

A solution of trifluoroacetic acid (12.25 mL, 159 mmol) in dichloromethane (10 mL) was added to a stirred, cooled (ice-bath) solution of tert-butyl 1-(ethylsulfonyl)-4-methylpiperidin-3-yl(methyl)carbamate (Preparation 33c, 3.0 g, 9.4 mmol) in dichloromethane (22 mL). The mixture was warmed to room temperature and stirred overnight. The solvent was evaporated and the residue was taken up in water and basified with 2M aqueous sodium hydroxide solution and then extracted with dichloromethane. The organic solvent was removed under reduced pressure to give the title compound (2.06 g, 97%) as an oil.

LRMS (m/z): 221 (M+1)⁺.

¹H-NMR δ (CDCl₃): 1.24 (d, 3H), 1.63 (t, 3H), 1.79-1.91 (m, 2H), 2.25-2.36 (m, 1H), 2.81 (bs, 1H), 2.90 (q, 2H), 3.06 (bs, 1H), 3.21-3.37 (m, 1H), 3.42-3.51 (m, 1H), 3.68 (bs, 2H), 3.91 (s, 3H)

### Preparation 34

### Cis-3-(3-amino-4-methylpiperidin-1-yl)-3-oxopropanenitrile

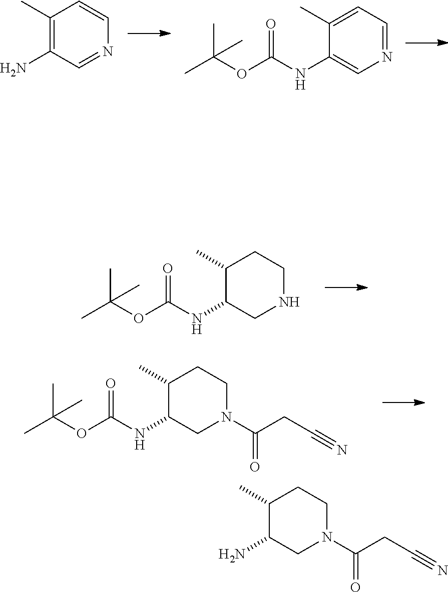

### a) tert-Butyl (4-methylpyridin-3-yl)carbamate

Sodium bis(trimethylsilyl)amide (1M in tetrahydrofuran, 32.0 mL, 32.0 mmol) was added dropwise to a cooled (ice-bath) solution of 4-methylpyridin-3-amine (1.72 g, 15.9 mmol) in tetrahydrofuran (30 mL). The mixture was warmed to ambient temperature and stirred for 5 minutes. The mixture was again cooled to 0° C. and a solution of di-tert-butyl dicarbonate (3.09 g, 14.2 mmol) in tetrahydrofuran was added. The mixture was warmed to ambient temperature and stirred for 18 hours. The pH of the mixture was adjusted to 5-6 using 0.1M aqueous hydrochloric acid. The organic layer was dried (MgSO₄) and evaporated to give the title compound (2.16 g, 70%) as a dark oil.

LRMS (m/z): 209 (M+1)⁺.

¹H-NMR δ (CDCl₃): 1.43 (s, 9H), 2.27 (s, 3H), 6.25 (bs, 1H), 7.10 (d, 1H), 8.23 (d, 1H), 8.86 (s, 1H).

### b) Cis-tert-butyl (4-methylpiperidin-3-yl)carbamate

To a solution of tert-butyl (4-methylpyridin-3-yl)carbamate (Preparation 34a, 1.9 g, 9.2 mmol) in methanol (15 mL) was added 5% rhodium on carbon (1.0 g). The mixture was hydrogenated (pressure of 60 psi) at 50° C. for 48 hours. The catalyst was filtered through Celite® and the filter cake washed with methanol. The filtrate and washings were combined and concentrated under reduced pressure and the residue was purified by flash chromatography (98:2:0.2 to 90:10:1 dichloromethane/methanol/ammonia) to give the title compound (0.60 g, 31%) as a pale yellow solid.

LRMS (m/z): 215 (M+1)⁺.

¹H-NMR δ (CDCl₃): 0.91 (d, 3H), 1.28 (d, 2H), 1.45 (s, 9H), 1.71 (bs, 5H), 2.59 (t, 1H), 2.72 (d, 1H), 2.96 (d, 2H), 3.68 (d, 1H), 5.30 (d, 1H).

### c) Cis-tert-butyl [1-(cyanoacetyl)-4-methylpiperidin-3-yl]carbamate

Triethylamine (0.26 mL, 1.8 mmol) and 3-[(2,5-dioxopyrrolidin-1-yl)oxy]-3-oxopropanenitrile (prepared as described in BE875054(A1), 0.364 g, 2.0 mmol) were added sequentially to a stirred solution of cis-tert-butyl 4-methylpiperidin-3-ylcarbamate (Preparation 34b, 0.36 g, 1.7 mmol) in dichloromethane (6 mL). The mixture was stirred at ambient temperature for 24 hours and then evaporated. Water was added and the mixture was extracted with dichloromethane. The organic layer was dried (MgSO₄) and evaporated and the residue was purified by flash chromatography (98:2 dichloromethane/methanol) to give the title compound (0.39 g, 83%) as a pale yellow oil.

LRMS (m/z): 282 (M+1)⁺

¹H-NMR δ (CDCl₃): 0.99 (d, 3H), 1.29 (qd, 1H), 1.46 (s, 9H), 2.01-1.85 (m, 1H), 2.69 (td, 1H), 3.17 (d, 1H), 3.46 (s, 2H), 3.71-3.62 (m, 1H), 3.87 (d, 1H), 4.51 (t, 2H).

### d) Cis-3-(3-amino-4-methylpiperidin-1-yl)-3-oxopropanenitrile

Trifluoroacetic acid (2.0 mL, 26 mmol) was added dropwise to a solution of cis-tert-butyl [1-(cyanoacetyl)-4-methylpiperidin-3-yl]carbamate (Preparation 34c, 0.39 g, 1.4 mmol) in dichloromethane (7 mL) and the mixture was stirred at room temperature for 1 hour. The mixture was then evaporated and taken up in water. The mixture was then taken to neutral pH with saturated aqueous sodium hydrogencarbonate solution and extracted with dichloromethane. The organic layer was dried (MgSO₄) and evaporated and the residue was purified by flash chromatography (98:2 to 90:10 dichloromethane/methanol) to give the title compound (0.180 g, 72%) as a pale yellow oil.

LRMS (m/z): 182 (M+1)⁺.

¹H-NMR δ (CDCl₃): 0.96 (d, 3H), 1.58-1.48 (m, 1H), 1.90-1.76 (m, 2H), 2.73 (ddd, 1H), 3.03-2.88 (m, 1H), 3.32-3.24 (m, 1H), 3.64 (d, 2H), 3.88-3.76 (m, 1H), 4.47-4.22 (m, 1H).

### Preparation 35

### Cis-1-(ethylsulfonyl)-4-methylpiperidin-3-amine

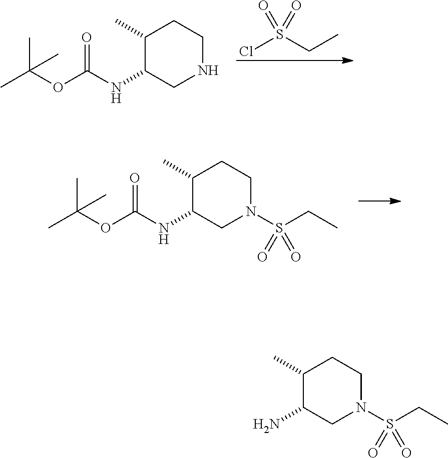

### a) Cis-tert-butyl [1-(ethylsulfonyl)-4-methylpiperidin-3-yl]carbamate

Triethylamine (0.33 mL, 2.3 mmol) and ethanesulfonyl chloride (0.17 mL, 1.7 mmol) were added sequentially to a cooled (ice-bath) solution of cis-tert-butyl (4-methylpiperidin-3-yl)carbamate (Preparation 34b, 0.25 g, 1.2 mmol) in dichloromethane (3 mL). The mixture was warmed to ambient temperature and stirred for 18 hours. Water was added and the organic layer was washed with water and brine, dried (MgSO₄) and evaporated. The residue was purified by flash chromatography (98:2 dichloromethane/methanol) to give the title compound (0.31 g, 85%) as a brown solid.

LRMS (m/z): 307 (M+1)⁺.

¹H-NMR δ (CDCl₃): 0.96 (d, 3H), 1.44 (s, 12H), 1.62 (s, 1H), 1.80-1.66 (m, 1H), 2.74 (t, 1H), 2.93 (dt, 3H), 3.49 (d, 1H), 3.80 (dd, 3H), 4.95 (d, 1H).

### b) Cis-1-(ethylsulfonyl)-4-methylpiperidin-3-amine

Cis-tert-butyl [1-(ethylsulfonyl)-4-methylpiperidin-3-yl]carbamate (Preparation 35a, 0.305 g, 1.0 mmol) was reacted according to the experimental procedure as described in Preparation 34d. The crude mixture was evaporated, water was added and the mixture was then basified with saturated aqueous sodium hydrogencarbonate solution to neutral pH. The aqueous layer was extracted with dichloromethane and the organic layer was dried (MgSO₄) and evaporated to give the title compound (0.145 g, 70%) as an oil.

LRMS (m/z): 207 (M+1)⁺.

¹H-NMR δ (CDCl₃): 1.01 (d, 3H), 1.45-1.22 (m, 3H), 1.60 (d, 4H), 2.85-2.73 (m, 1H), 3.13-2.85 (m, 3H), 3.86-3.47 (m, 2H).

### Preparation 36

### 1-((2S,4S)-2-cyano-4-fluoropyrrolidine-1-carbonyl)-3-methyl-1H-imidazol-3-ium iodide

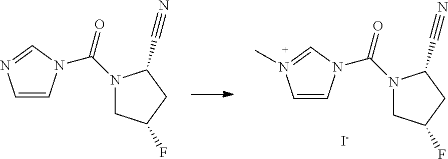

Methyl iodide (0.075 mL, 1.2 mmol) was added to a stirred solution of (2S,4S)-4-fluoro-1-(1H-imidazole-1-carbonyl)pyrrolidine-2-carbonitrile (0.29 mmol, prepared as described in US2005256310(A1)) in acetonitrile (1 mL) and the reaction mixture was stirred at ambient temperature overnight. The solvent was removed under reduced pressure to give the title compound as a solid (99%), which was used without further purification.

LRMS (m/z): 223 (M−1)⁻.

### Preparation 37

### 1-((Cyanomethyl)(methyl)carbamoyl)-3-methyl-1H-imidazol-3-ium iodide

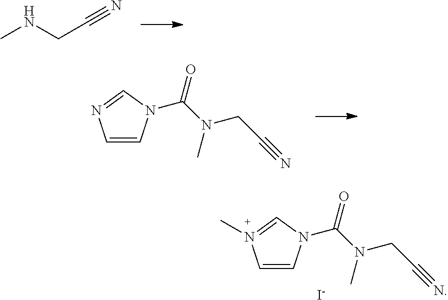

### a) N-(cyanomethyl)-N-methyl-1H-imidazole-1-carboxamide

2-(Methylamino)acetonitrile (1 g, 14.3 mmol) was reacted with 1,1′-carbonyldiimidazole (2.5 g, 15.4 mmol) according to the experimental procedure as described in US2005256310(A1). The crude product was purified by flash chromatography (4:1, dichloromethane/methanol) to give the title compound (0.24 g, 10%) as an oil.

LRMS (m/z): 165 (M+1)⁺.

¹H-NMR δ (CDCl₃): 3.28 (s, 3H), 4.37 (s, 2H), 7.15 (s, 1H), 7.30 (bs, 1H), 7.97 (s, 1H)

### b) 1-((Cyanomethyl)(methyl)carbamoyl)-3-methyl-1H-imidazol-3-ium iodide

Obtained as an oil (45%) from N-(cyanomethyl)-N-methyl-1H-imidazole-1-carboxamide (Preparation 37a, 0.24 g, 1.46 mmol) and methyl iodide (0.36 mL, 5.9 mmol) following the experimental procedure as described in Preparation 36. The isolated crude product was used without further purification.

LRMS (m/z): 179 (M−1)⁻.

¹H-NMR δ (DMSO-d₆): 3.34 (s, 3H), 3.91 (s, 2H), 4.64 (s, 3H), 7.06 (bs, 1H), 7.58 (bs, 1H), 8.09 (s, 1H)

### Preparation 38

### 2-Methoxy-4-(4,4,5,5-tetramethyl-1,3,2-dioxaborolan-2-yl)pyridine

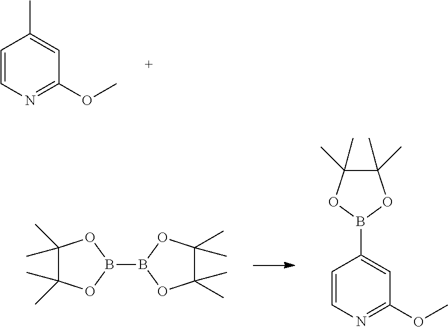

Nitrogen was bubbled for 5 minutes through a mixture of 4-iodo-2-methoxypyridine (0.099 g, 0.42 mmol), bis(pinacolato)diboron (0.12 g, 0.47 mmol) and potassium acetate (0.13 g, 1.31 mmol) in N,N′-dimethylformamide (1 mL) contained in a microwave vessel. Then [1,1-bis(diphenylphosphino)ferrocene] dichloropalladium (II) complex with dichloromethane (1:1) (0.017 g, 0.02 mmol) was added and the vessel was sealed and subjected to microwave irradiation for 4 hours at 80° C. The reaction mixture was diluted with ethyl acetate and filtered through Celite®. The filtrate was washed with saturated aqueous sodium hydrogencarbonate solution, water, brine, dried (MgSO₄) and evaporated to give the title compound (0.128 g, 82%) as a brown oil which was used without further purification.

LRMS (m/z): 236 (M+1)⁺.

### Preparation 39

### 1H-pyrazole-3-carboxylic acid

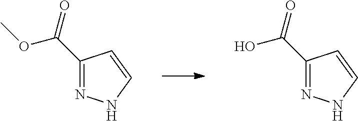

A 2M aqueous sodium hydroxide solution (1.8 mL, 3.6 mmol) was added to a solution of ethyl 1H-pyrazole-4-carboxylate (0.2 g, 1.55 mmol) in ethanol (4 mL). The reaction mixture was stirred at room temperature overnight and at 80° C. for 2 further hours. The ethanol was evaporated and the mixture was neutralized to give a precipitate which was filtered and dried to give the title compound (0.089 g, 51%) as a white solid.

LRMS (m/z): 113 (M+1)⁺.

### Preparation 40

### (E)-4-Aminotricyclo[3.3.1.1^{3,7}]decan-1-ol

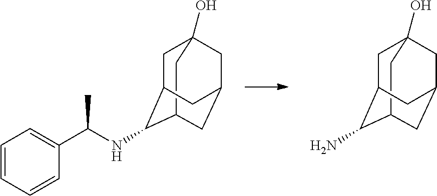

To a solution of 4-{[(1R)-1-phenylethyl]amino}adamantan-1-ol (0.400 g, 1.47 mmol; prepared as described in Tetrahedron. Lett. 2006, 47, 8063) in ethanol (58 mL) was added 10% palladium on charcoal (0.033 g) and ammonium formate (0.430 g, 6.82 mmol). The reaction mixture was stirred at 85° C. for one hour then filtered through Celite® and the filtrate was evaporated to dryness to obtain the title compound as a solid (99%) which was used in the next step without further purification.

E-isomer: ¹H-NMR δ (CDCl₃): 1.17-1.52 (m, 5H), 1.7-1.83 (m, 6H), 1.9 (m, 3H), 1.94 (m, 1H), 2.09 (m, 1H), 3.01 (bs, 1H).

### Preparation 41

### (Z)-4-Aminotricyclo[3.3.1.1^{3,7}]decan-1-ol

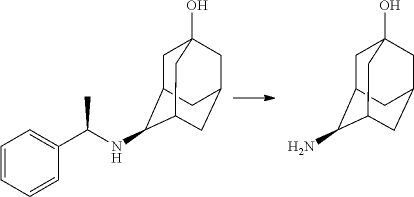

The title compound was obtained (99%) from the corresponding Z-Isomer (0.102 g, 0.38 mmol; prepared as described in Tetrahedron. Lett. 2006, 47, 8063;) following the experimental procedure as described in Preparation 40. The crude product was used in the next step without further purification.

Z-isomer: ¹H-NMR δ (CDCl₃): 1.51-1.4 (m, 2H), 1.7-1.57 (m, 6H), 1.9-1.8 (m, 4H), 2.09-2.00 (m, 1H), 2.87 (bs, 1H).

### Preparation 42

### 3-(4,5-Dichloropyrimidin-2-yl)-6-fluoroimidazo[1,2-a]pyridine

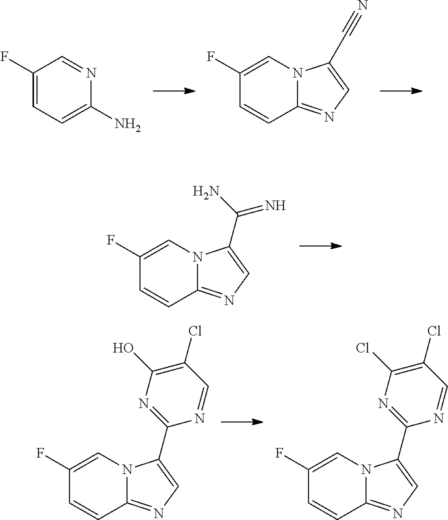

### a) 6-Fluoroimidazo[1,2-a]pyridine-3-carbonitrile

1-Bromopyrrolidine-2,5-dione (15.8 g, 89.2 mmol) was added portionwise to a cooled (ice-bath), stirred solution of (E)-3-methoxyacrylonitrile (7.49 mL, 89.2 mmol) in 1,4-dioxane/water (3:1, 600 mL). The solution was stirred for 30 minutes at 0° C. and then 5-fluoropyridin-2-amine (5.0 g, 44.6 mmol) was added. The mixture was warmed to ambient temperature over 2 hours and then was heated to 60° C. and stirred for 16 hours at 60° C. The organic solvent was removed under reduced pressure and the residue was partitioned between ethyl acetate and aqueous sodium hydrogencarbonate solution. The organic phase was dried, filtered and evaporated to dryness. The crude product was purified by flash chromatography (15-50% ethyl acetate/hexane) to give the title compound (94%) as a brown solid.

LRMS (m/z): 162 (M+1)⁺.

¹H-NMR δ (DMSO-d₆): 7.71 (ddd, 1H), 7.93 (dd, 1H), 8.49 (s, 1H), 8.98 (bs, 1H).

### b) 6-Fluoroimidazo[1,2-a]pyridine-3-carboximidamide

To a solution of 6-fluoroimidazo[1,2-a]pyridine-3-carbonitrile (5.6 g, 34.8 mmol, Preparation 42a) in MeOH (80 mL) was added sodium methoxide (0.19 g, 3.4 mmol). The mixture was stirred at ambient temperature for 17 hours, then ammonium chloride (2.05 g, 38.3 mmol) was added and the mixture was refluxed for 4 hours. The solvent was removed under reduced pressure and the resulting solid was washed with ethyl acetate (8×30 mL). The title compound was obtained as a brown solid (58%) and was used in the next step without further purification.

LRMS (m/z): 179 (M+1)⁺. δ

¹H-NMR δ (DMSO-d₆): 7.70 (dd, 1H), 7.89 (dd, 1H), 8.35 (s, 1H), 8.85 (brs, 1H).

### c) 5-Chloro-2-(6-fluoroimidazo[1,2-a]pyridin-3-yl)pyrimidin-4-ol

To a solution of sodium (0.9 g, 39.1 mmol) in methanol (50 mL) was added 6-fluoroimidazo[1,2-a]pyridine-3-carboximidamide (3.5 g, 19.6 mmol, Preparation 42b) and ethyl 2-chloro-3-oxopropanoate (5.8 g, 38.5 mmol). The mixture was refluxed under strong agitation for 18 hours. The reaction mixture was cooled to ambient temperature and filtered. The resulting solid was washed with methanol and ether to yield the title compound (70%), which was used in the next step without further purification.

LRMS (m/z): 265 (M+1)⁺.

¹H-NMR δ (DMSO-d₆): 7.43 (dd, 1H), 7.73 (dd, 1H), 7.95 (s, 1H), 8.26 (s, 1H), 10.18 (dd, 1H).

### d) 3-(4,5-Dichloropyrimidin-2-yl)-6-fluoroimidazo[1,2-a]pyridine

A solution of 5-chloro-2-(6-fluoroimidazo[1,2-a]pyridin-3-yl)pyrimidin-4-ol (2.6 g, 9.8 mmol, Preparation 42c) in phosphoryl trichloride (26 mL) was stirred at 110° C. for 3 hours. The crude reaction was slowly added onto water, basified with 8N sodium hydroxide and extracted with ethyl acetate. The organic phase was washed with water and brine and the organic solvent was removed under reduced pressure to give the title compound (62%) as a solid.

LRMS (m/z): 284 (M+1)⁺.

¹H-NMR δ (CDCl₃): 7.34 (ddd, 1H), 7.75 (dd, 1H), 8.66 (s, 1H), 8.70 (s, 1H), 9.75 (dd, 1H).

### Preparation 43

### 5-Chloro-2-(6-fluoroimidazo[1,2-a]pyridin-3-yl)-N-[(3R)-piperidin-3-yl]pyrimidin-4-amine

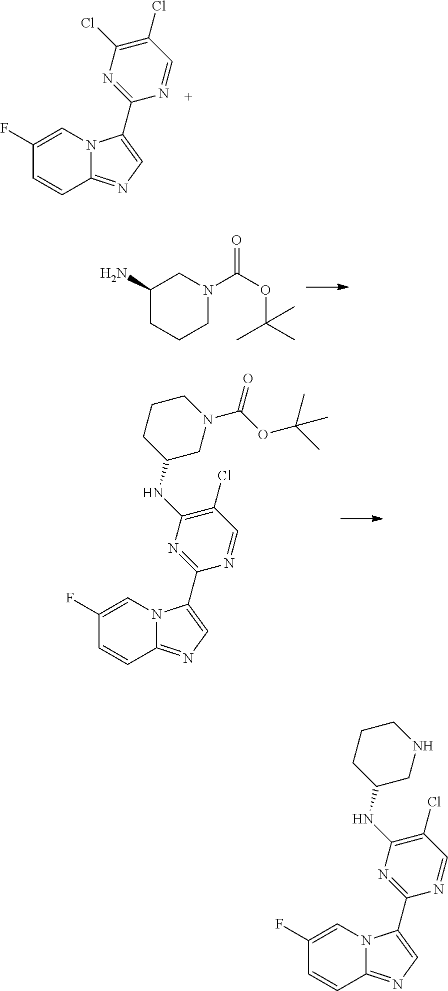

### a) tert-Butyl (3R)-3-{[5-chloro-2-(6-fluoroimidazo[1,2-a]pyridin-3-yl)pyrimidin-4-yl]amino}piperidine-1-carboxylate

A solution of 3-(4,5-dichloropyrimidin-2-yl)-6-fluoroimidazo[1,2-a]pyridine (0.52 g, 1.84 mmol, Preparation 42d) and (R)-tert-butyl 3-aminopiperidine-1-carboxylate (1.12 g, 5.59 mmol) in ethanol (40 mL) was refluxed for 18 hours. The organic solvent was removed under reduced pressure. The residue obtained was triturated with ether and evaporated to dryness to obtain the title compound (99%) as an oil.

LRMS (m/z): 447 (M+1)⁺.

### b) 5-Chloro-2-(6-fluoroimidazo[1,2-a]pyridin-3-yl)-N-[(3R)-piperidin-3-yl]pyrimidin-4-amine

Obtained as a solid (40%) from tert-butyl (3R)-3-{[5-chloro-2-(6-fluoroimidazo[1,2-a]pyridin-3-yl)pyrimidin-4-yl]amino}piperidine-1-carboxylate (Preparation 43a) following the experimental procedure as described in Preparation 5b.

LRMS (m/z): 347 (M+1)⁺.

¹H-NMR δ (CDCl₃): 1.64 (bs, 1H), 1.80 (dd, 2H), 1.92-2.00 (m, 1H), 2.81-2.92 (m, 3H), 3.24 (dd, 1H), 4.26-4.35 (m, 2H), 5.92 (bs, 1H), 7.23 (dd, 1H), 7.67 (dd, 1H), 8.22 (s, 1H), 8.51 (s, 1H), 9.87 (dd, 1H).

### Preparation 44

### 3-(4-Chloropyrimidin-2-yl)-6-fluoroimidazo[1,2-a]pyridine

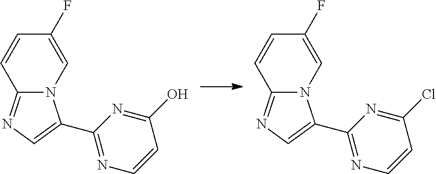 [SMILES: Oc1ccnc(-c2cnc3ccc(F)cn23)n1 ; Fc1ccc2ncc(-c3nccc(Cl)n3)n2c1]

A mixture of 2-(6-fluoroimidazo[1,2-a]pyridin-3-yl)pyrimidin-4-ol (0.95 g, 4.13 mmol, Preparation 8) and phosphorous oxychloride (8.31 mL, 90.78 mmol) was heated to 110° C. during 5 h. After elimination of the excess of phosphorous oxychloride under reduced pressure, the residue was dissolved in ethyl acetate and washed successively with water, 4% aqueous hydrogen carbonate solution and brine. The organic phase was dried over sodium sulphate, filtered and the solvent was removed under reduced pressure to give the title compound (0.45 g, 44%) as a solid.

LRMS (m/z): 249 (M+1)⁺.

¹H-NMR δ (CDCl₃): 7.16 (d, 1H), 7.35 (ddd, 1H), 7.79 (dd, 1H), 8.66 (d, 1H), 8.69 (s, 1H), 9.89 (dd, 1H).

### Preparation 45

### (S)-1-(2-(6-Fluoroimidazo[1,2-a]pyridin-3-yl)pyrimidin-4-yl)pyrrolidine-2-carboxylic acid

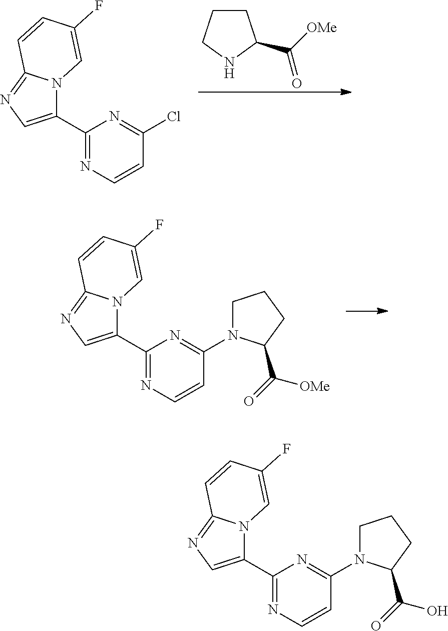 [SMILES: Fc1ccc2ncc(-c3nccc(Cl)n3)n2c1 ; COC(=O)[C@@H]1CCCN1 ; COC(=O)[C@@H]1CCCN1c1ccnc(-c2cnc3ccc(F)cn23)n1 ; O=C(O)[C@@H]1CCCN1c1ccnc(-c2cnc3ccc(F)cn23)n1]

### a) (S)-Methyl 1-(2-(6-fluoroimidazo[1,2-a]pyridin-3-yl)pyrimidin-4-yl)pyrrolidine-2-carboxylate

A mixture of 3-(4-chloropyrimidin-2-yl)-6-fluoroimidazo[1,2-a]pyridine (140 mg, 0.56 mmol, Preparation 44), (S)-methylpyrrolidine-2-carboxylate hydrochloride (140 mg, 0.85 mmol) and triethylamine (200 μL, 1.43 mmol) in methanol (10 mL) was heated to reflux overnight. The solvent was evaporated under reduced pressure and the residue partitioned between methylene chloride and water. The organic layer was separated, washed with water, 4% aqueous hydrogen carbonate solution and brine, dried over sodium sulphate and the solvent removed under reduced pressure. The resulting residue was purified by flash chromatography (dichloromethane to dichloromethane/methanol 92:8) to obtain the title compound (160 mg, 83%) as a solid.

LRMS (m/z): 341 (M⁺).

¹H-NMR δ (CDCl₃): 2.14 (td, 3H), 2.37 (dd, 1H), 3.42-3.60 (m, 1H), 3.62-3.71 (m, 1H), 3.76 (s, 3H), 4.81 (bs, 1H), 6.26 (bs, 1H), 7.22 (dd, 1H), 7.66 (dd, 1H), 8.31 (d, 1H), 8.50 (s, 1H), 9.90 (bs, 1H).

### b) (S)-1-(2-(6-Fluoroimidazo[1,2-a]pyridin-3-yl)pyrimidin-4-yl)pyrrolidine-2-carboxylic acid

A mixture of (S)-methyl 1-(2-(6-fluoroimidazo[1,2-a]pyridin-3-yl)pyrimidin-4-yl)pyrrolidine-2-carboxylate (122 mg, 0.36 mmol, Preparation 45a) and aqueous sodium hydroxide solution (2N, 536 μL, 1.07 mmol) in methanol (10 mL) was heated to 50° C. until completion of the reaction. The solvent was evaporated under reduced pressure and the residue was suspended in water, neutralised with 2N aqueous hydrochloric acid solution, filtered and purified by reverse phase chromatography (C-18 silica from Waters, water/1:1 acetonitrile-methanol as eluents [0.1% v/v formic acid buffered] 0% to 100%) to yield the title compound (98 mg, 84%) as a solid.

LRMS (m/z): 327 (M⁺).

¹H-NMR δ (CDCl₃): 2.14 (ddd, 1H), 2.25-2.57 (m, 3H), 3.45-3.63 (m, 1H), 3.69 (t, 1H), 4.67 (dd, 1H), 6.28 (d, 1H), 7.18 (ddd, 1H), 8.01 (dd, 1H), 8.24 (d; 1H), 8.74 (s, 1H), 10.02 (dd, 1H).

### Preparation 46

### (R)-1-(2-(6-Fluoroimidazo[1,2-a]pyridin-3-yl)pyrimidin-4-yl)pyrrolidine-2-carboxylic acid

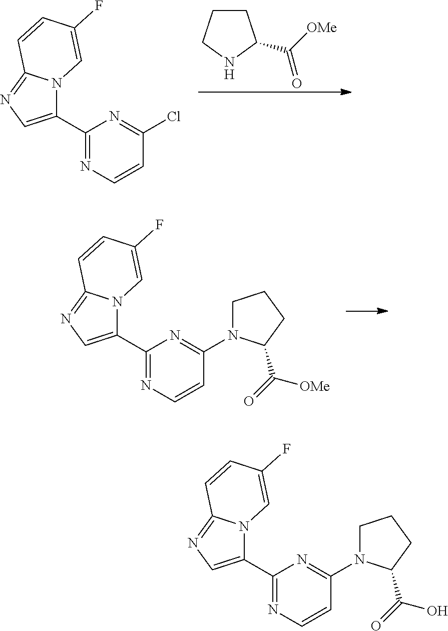 [SMILES: Fc1ccc2ncc(-c3nccc(Cl)n3)n2c1 ; COC(=O)[C@H]1CCCN1 ; COC(=O)[C@H]1CCCN1c1ccnc(-c2cnc3ccc(F)cn23)n1 ; O=C(O)[C@H]1CCCN1c1ccnc(-c2cnc3ccc(F)cn23)n1]

### a) (R)-Methyl 1-(2-(6-fluoroimidazo[1,2-a]pyridin-3-yl)pyrimidin-4-yl)pyrrolidine-2-carboxylate

A mixture of 3-(4-chloropyrimidin-2-yl)-6-fluoroimidazo[1,2-a]pyridine (140 mg, 0.56 mmol, Preparation 44), (R)-methylpyrrolidine-2-carboxylate hydrochloride (140 mg, 0.85 mmol) and triethylamine (200 μL, 1.43 mmol) in methanol (10 mL) was heated to reflux overnight. The solvent was evaporated under reduced pressure and the residue partitioned between methylene chloride and water. The organic layer was separated, washed with water, 4% aqueous hydrogen carbonate solution and brine, dried over sodium sulphate and the solvent removed under reduced pressure. The resulting residue was purified by flash chromatography (dichloromethane to dichloromethane/methanol 92:8) to yield the title compound (42 mg, 22%) as a solid.

LRMS (m/z): 341 (M⁺).

¹H-NMR δ (CDCl₃): 2.15 (m, 3H), 2.31-2.47 (m, 1H), 3.42-3.59 (m, 1H), 3.64-3.74 (m, 1H), 3.76 (s, 3H), 4.80 (bs, 1H), 6.27 (bs, 1H), 7.24 (t, 1H), 7.71 (dd, 1H), 8.31 (d, 1H), 8.50 (s, 1H), 9.90 (bs, 1H).

### b) (R)-1-(2-(6-Fluoroimidazo[1,2-a]pyridin-3-yl)pyrimidin-4-yl)pyrrolidine-2-carboxylic acid

Obtained as a white solid (22%) from (R)-methyl 1-(2-(6-fluoroimidazo[1,2-a]pyridin-3-yl)pyrimidin-4-yl)pyrrolidine-2-carboxylate (10 mg, 0.03 mmol, Preparation 46a) following the experimental procedure as described in Preparation 45b.

LRMS (m/z): 327 (M⁺).

¹H-NMR δ (CDCl₃): 2.15 (d, 2H), 2.25-2.55 (m, 4H), 3.46-3.62 (m, 2H), 3.63-3.77 (m, 1H), 4.67 (dd, 2H), 6.28 (d, 1H), 7.18 (ddd, 1H), 8.00 (dd, 1H), 8.25 (d, 1H), 8.74 (s, 1H), 10.03 (dd, 1H).

### Preparation 47

### 2-((1r,4r)-4-Aminocyclohexyl)acetonitrile

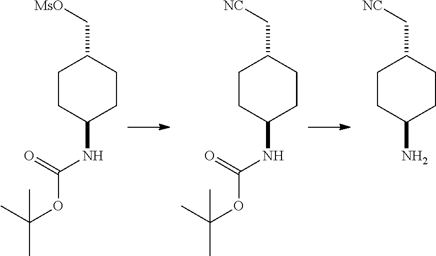 [SMILES: CC(C)(C)OC(=O)N[C@H]1CC[C@H](COS(C)(=O)=O)CC1 ; CC(C)(C)OC(=O)N[C@H]1CC[C@H](CC#N)CC1 ; N#CC[C@H]1CC[C@H](N)CC1]

### a) tert-Butyl (1r,4r)-4-(cyanomethyl)cyclohexylcarbamate

A mixture of ((1r,4r)-4-(tert-butoxycarbonylamino)cyclohexyl)methyl methanesulfonate (70 mg, 0.18 mmol, prepared as described in WO2005/87761) and sodium cyanide (30 mg, 0.61 mmol) in DMSO (1 mL) was heated overnight at 55° C. The reaction mixture was diluted in ethyl acetate, washed successively with 4% aqueous sodium hydrogencarbonate solution, water and brine, dried over magnesium sulphate, filtered and the solvent evaporated under reduced pressure. The crude residue (50 mg, 100%) was used in the next synthetic step without further purification.

¹H-NMR δ (CDCl₃): 0.99-1.30 (m, 4H), 1.44 (s, 9H), 1.55-1.71 (m, 1H), 1.91 (d, 2H), 2.06 (d, 2H), 2.26 (d, 2H), 3.31-3.43 (m, 1H), 4.39 (bs, 1H).

### b) 2-((1r,4r)-4-Aminocyclohexyl)acetonitrile hydrochloride

Tert-butyl (1r,4r)-4-(cyanomethyl)cyclohexylcarbamate (Preparation 47a, 0.348 g, 1.46 mmol) was added to a 4M hydrogen chloride solution in dioxane (3.65 mL) and the resulting solution was stirred overnight at room temperature. The solvent was evaporated in vacuo and the residue was treated with diethyl ether. The resultant suspension was filtered to give the title compound (0.226 g, 89%) as a white solid.

LRMS (m/z): 139 (M+H)⁺.

¹H-NMR δ (DMSO-d₆): 1.14 (ddd, 2H), 1.37 (ddd, 2H), 1.60 (m, 1H), 1.83 (d, 2H), 1.99 (d, 2H), 2.50 (d, 2H), 2.94 (m, 1H), 8.08 (br s, 2H).

### Preparation 48

### (R)-2-(6-Fluoroimidazo[1,2-a]pyridin-3-yl)-5-methyl-N-(pyrrolidin-3-yl)pyrimidin-4-amine

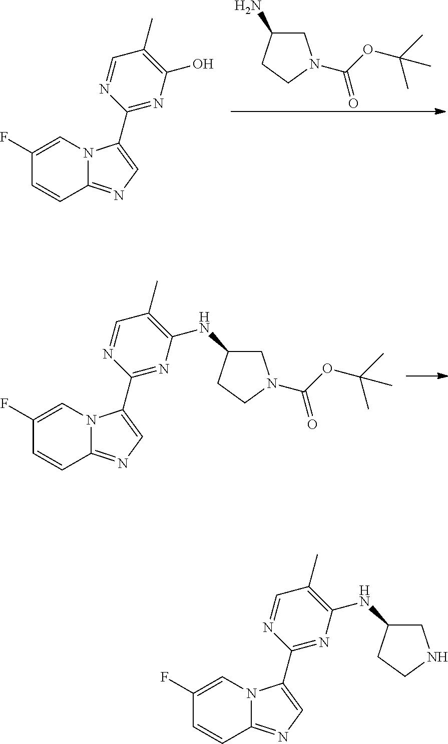 [SMILES: Cc1cnc(-c2cnc3ccc(F)cn23)nc1O ; CC(C)(C)OC(=O)N1CC[C@@H](N)C1 ; Cc1cnc(-c2cnc3ccc(F)cn23)nc1N[C@@H]1CCN(C(=O)OC(C)(C)C)C1 ; Cc1cnc(-c2cnc3ccc(F)cn23)nc1N[C@@H]1CCNC1]

### a) (R)-tert-Butyl 3-(2-(6-fluoroimidazo[1,2-a]pyridin-3-yl)-5-methylpyrimidin-4-ylamino)pyrrolidine-1-carboxylate

Obtained as an oil (263 mg, 45%) from 2-(6-fluoroimidazo[1,2-a]pyridin-3-yl)-5-methylpyrimidin-4-ol (350 mg, 1.43 mmol, Preparation 19b) and (R)-tert-butyl 3-aminopyrrolidine-1-carboxylate (533 mg, 2.86 mmol, prepared as described in US2009/2215665) following the experimental procedure as described in Preparation 5a.

LRMS (m/z): 413 (M+1)⁺.

¹H-NMR δ (CDCl₃): 1.42-1.53 (m, 2H), 1.60 (s, 9H), 2.37 (bs, 1H), 2.64 (s, 3H), 3.57 (bs, 2H), 3.85 (bs, 1H), 4.64 (s, 1H), 4.81 (bs, 1H), 7.22 (dd, 1H), 7.68 (dd, 1H), 8.10 (s, 1H), 8.51 (s, 1H), 9.98 (dd, 1H).

### b) (R)-2-(6-Fluoroimidazo[1,2-a]pyridin-3-yl)-5-methyl-N-(pyrrolidin-3-yl)pyrimidin-4-amine

Obtained as a white solid (156 mg, 78%) from (R)-tert-butyl 3-(2-(6-fluoroimidazo[1,2-a]pyridin-3-yl)-5-methylpyrimidin-4-ylamino)pyrrolidine-1-carboxylate (263 mg, 0.64 mmol, Preparation 48a) and trifluoroacetic acid (245 μL, 3.18 mmol) following the experimental procedure as described in Preparation 5b.

LRMS (m/z): 313 (M+1)⁺.

¹H-NMR δ (CDCl₃): 1.74-1.87 (m, 2H), 2.08 (s, 3H), 2.37 (ddd, 1H), 2.92-3.10 (m, 2H), 3.16 (dt, 1H), 3.36 (dd, 1H), 4.72 (dt, 1H), 4.82 (bs, 1H), 7.21 (ddd, 1H), 7.67 (dd, 1H), 8.06 (s, 1H), 8.51 (s, 1H), 10.01 (dd, 1H).

### Preparation 49

### 2-(6-Fluoroimidazo[1,2-a]pyridin-3-yl)-N-((5-fluoropyridin-2-yl)methyl)-5-nitropyrimidin-4-amine

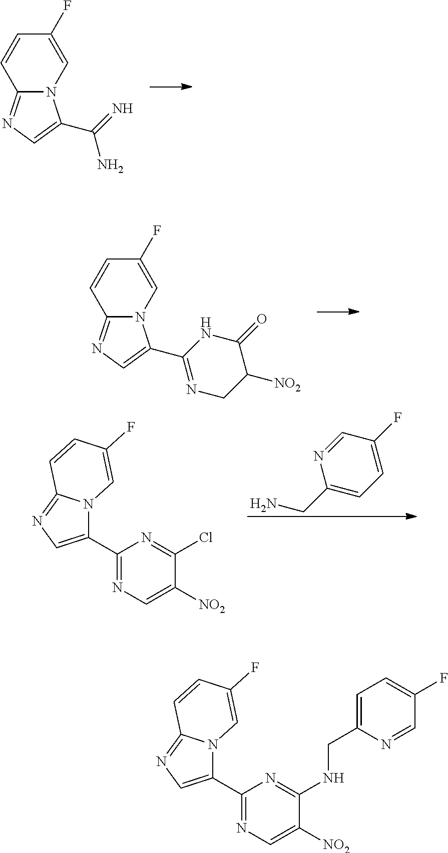 [SMILES: N=C(N)c1cnc2ccc(F)cn12 ; O=C1NC(c2cnc3ccc(F)cn23)=NCC1[N+](=O)[O-] ; O=[N+]([O-])c1cnc(-c2cnc3ccc(F)cn23)nc1Cl ; NCc1ccc(F)cn1 ; O=[N+]([O-])c1cnc(-c2cnc3ccc(F)cn23)nc1NCc1ccc(F)cn1]

### a) 2-(6-Fluoroimidazo[1,2-a]pyridin-3-yl)-5-nitropyrimidin-4(3H)-one

A mixture of 6-fluoroimidazo[1,2-a]pyridine-3-carboximidamide (Preparation 42b, 1.03 g, 5.75 mmol), (Z)-ethyl 3-(dimethylamino)-2-nitroacrylate (3.0 g, 15.9 mmol) and triethylamine (4.8 mL, 34.4 mmol) in ethanol (15 mL) was heated to 90° C. in a sealed tube for 22 hours. The solvent was evaporated and the residue was partitioned between water and chloroform. The organic layer was separated and the aqueous layer washed several times with chloroform. The combined organic extracts were dried over magnesium sulphate, filtered and the solvent evaporated to give a semi-solid residue which was treated with aqueous saturated potassium carbonate solution to give a solid which was filtered, washed with water and dried to give the title compound (1.23 g, 78%) as a solid.

LRMS (m/z): 274 (M−1)⁺.

¹H-NMR δ (DMSO-d₆): 7.48-7.61 (m, 2H), 7.74-7.88 (m, 1H), 8.39 (br s, 1H), 8.79 (br s, 1H), 10.19 (br s, 1H).

### b) 3-(4-Chloro-5-nitropyrimidin-2-yl)-6-fluoroimidazo[1,2-a]pyridine

Phosphorous oxychloride (10 mL) was added to 2-(6-fluoroimidazo[1,2-a]pyridin-3-yl)-5-nitropyrimidin-4(31-1)-one (Preparation 49a, 1.12 g, 4.07 mmol) and the resulting suspension was stirred and heated to 90° C. in a sealed tube for 2 hours. The solvent was evaporated, taken up in dichloromethane and then neutralized to pH 7 by addition of an aqueous sodium hydrogencarbonate solution. The organic layer was separated and the aqueous layer was extracted several times with dichloromethane. The combined organic extracts were dried over magnesium sulphate, filtered and evaporated to give the title compound (0.81 g, 68%) as a yellow solid.

LRMS (m/z): 294 (M+1)⁺.

¹H-NMR δ (CDCl₃): 7.47 (ddd, 1H), 7.84 (ddd, 1H), 8.84 (s, 1H), 9.35 (s, 1H), 9.83 (ddd, 1H).

### c) 2-(6-Fluoroinnidazo[1,2-a]pyridin-3-yl)-N4(5-fluoropyridin-2-yl)methyl)-5-nitropyrimidin-4-amine

Diisopropylethylamine (742 μL, 4.26 mmol) and (5-fluoropyridin-2-yl)methanamine dihydrochloride (275 mg, 1.38 mmol) were added to a stirred suspension of 3-(4-chloro-5-nitropyrimidin-2-yl)-6-fluoroimidazo[1,2-a]pyridine (Preparation 49b, 250 mg, 0.85 mmol) in tetrahydrofuran (8 mL) and the mixture was stirred at room temperature overnight. The solvent was evaporated and water was added. The resulting suspension was filtered to give the title compound (309 mg, 93%) as a yellow solid.

LRMS (m/z): 384 (M+1)⁺.

¹H-NMR δ (DMSO-d₆): 5.01 (d, 2H), 7.55-7.74 (m, 3H), 7.87 (dd, 1H), 8.54 (d, 1H), 8.56 (s, 1H), 9.25 (s, 1H), 9.47 (dd, 1H), 9.73 (t, 1H).

### Preparation 50

### N-(2-(6-Fluoroimidazo[1,2-a]pyridin-3-yl)-5-nitropyrimidin-4-yl)-5,6,7,8-tetrahydroquinolin-5-amine

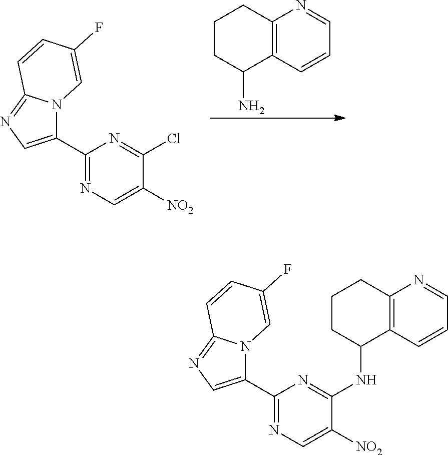 [SMILES: O=[N+]([O-])c1cnc(-c2cnc3ccc(F)cn23)nc1Cl ; NC1CCCc2ncccc21 ; O=[N+]([O-])c1cnc(-c2cnc3ccc(F)cn23)nc1NC1CCCc2ncccc21]

Obtained as a yellow solid (51%) from 3-(4-chloro-5-nitropyrimidin-2-yl)-6-fluoroimidazo[1,2-a]pyridine (Preparation 49b) and 5,6,7,8-tetrahydroquinolin-5-amine following the experimental procedure as described in Preparation 49c.

LRMS (m/z): 406 (M+1)⁺.

### Preparation 51

### (R)-2-(6-Fluoroimidazo[1,2-a]pyridin-3-yl)-N-(3-methylbutan-2-yl)-5-nitropyrimidin-4-amine

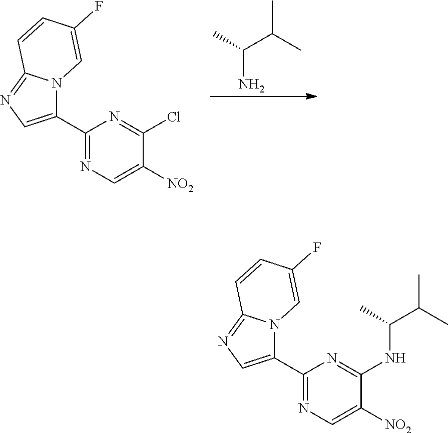

Obtained as a yellow solid (126 mg, 85%) from 3-(4-chloro-5-nitropyrimidin-2-yl)-6-fluoroimidazo[1,2-a]pyridine (Preparation 49b) and (R)-3-methylbutan-2-amine following the experimental procedure as described in Preparation 49c.

LRMS (m/z): 345 (M+1)⁺.

### Preparation 52

### 2-(6-Fluoroimidazo[1,2-a]pyridin-3-yl)-N-(1-methoxypropan-2-yl)-5-nitropyrimidin-4-amine

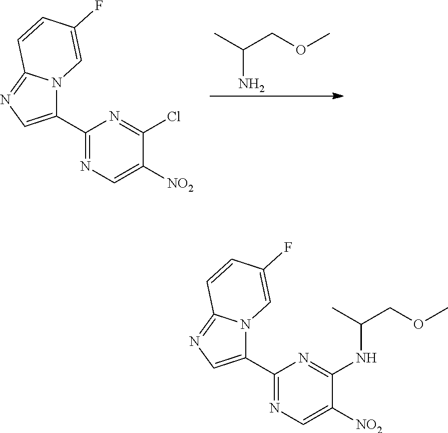

Obtained as a yellow solid (115 mg, 81%) from 3-(4-chloro-5-nitropyrimidin-2-yl)-6-fluoroimidazo[1,2-a]pyridine (120 mg, 0.41 mmol, see Preparation 49b) and commercially available (R)-3-methylbutan-2-amine (47 mg, 0.53 mmol) following the experimental procedure as described in Preparation 49c.

LRMS (m/z): 347 (M+1)⁺

### Preparation 53

### 2-((1r,4r)-4-(2-(6-Fluoroimidazo[1,2-a]pyridin-3-yl)-5-nitropyrimidin-4-ylamino)cyclohexyl)acetonitrile

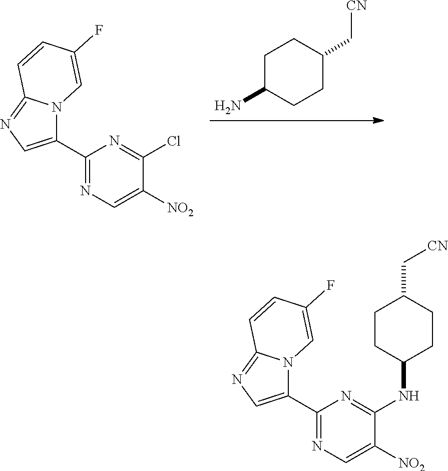

Obtained as a yellow solid (81%) from 3-(4-chloro-5-nitropyrimidin-2-yl)-6-fluoroimidazo[1,2-a]pyridine (Preparation 49b) and 2-((1r,4r)-4-aminocyclohexyl)acetonitrile hydrochloride (Preparation 47b) following the experimental procedure as described in Preparation 49c.

LRMS (m/z): 396 (M+1)⁺.

¹H-NMR δ (CDCl₃): 1.27 (d, 1H), 1.37-1.61 (m, 4H), 1.85 (m, 1H), 2.10 (d, 2H), 2.39 (m, 3H), 4.25 (m, 1H), 7.39 (br t, 1H), 7.78 (dd, 1H), 8.41 (d, 1H), 8.72 (s, 1H), 9.27 (s, 1H), 9.88 (m, 1H).

### Preparation 54

### (1r,4r)-4-(2-(6-Fluoroimidazo[1,2-a]pyridin-3-yl)-5-nitropyrimidin-4-ylamino)-1-methylcyclohexanol

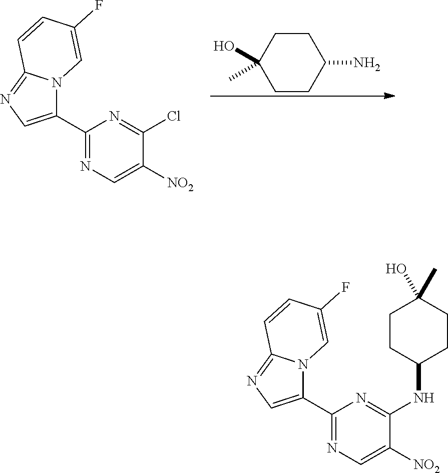

Obtained as a yellow solid (55%) from 3-(4-chloro-5-nitropyrimidin-2-yl)-6-fluoroimidazo[1,2-a]pyridine (110 mg, 0.37 mmol, see Preparation 49b) and (1r,4r)-4-amino-1-methylcyclohexanol (prepared as described in WO2006/76595) following the experimental procedure as described in Preparation 49c.

LRMS (m/z): 387 (M+1)⁺.

¹H-NMR δ (DMSO-d₆): 1.26 (bs, 4H), 1.64 (bs, 3H), 1.78 (m, 1H), 2.01 (m, 2H), 4.31 (m, 1H), 4.48 (s, 1H), 7.75 (m, 1H), 7.97 (m, 1H), 8.67 (s, 2H), 9.24 (s, 1H), 9.82 (m, 1H).

### Preparation 55

### 2-(6-Fluoroimidazo[1,2-a]pyridin-3-yl)-N-(1-(5-fluoropyridin-2-yl)-2-methoxyethyl)-5-nitropyrimidin-4-amine

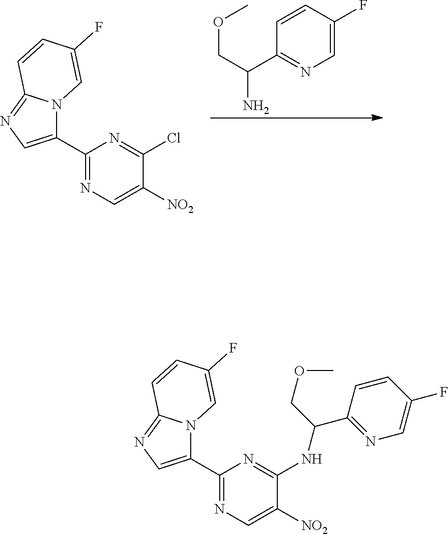 [SMILES: O=[N+]([O-])c1cnc(-c2cnc3ccc(F)cn23)nc1Cl ; COCC(N)c1ccc(F)cn1 ; COCC(Nc1nc(-c2cnc3ccc(F)cn23)ncc1[N+](=O)[O-])c1ccc(F)cn1]

Obtained as a yellow solid (85%) from 3-(4-chloro-5-nitropyrimidin-2-yl)-6-fluoroimidazo[1,2-a]pyridine (120 mg, 0.41 mmol, see Preparation 49b) and 1-(5-fluoropyridin-2-yl)-2-methoxyethanamine (76 mg, 0.45 mmol) following the experimental procedure as described in Preparation 49c.

LRMS (m/z): 428 (M+1)⁺.

¹H-NMR δ (DMSO-d₆): 3.34 (s, 3H), 3.85 (dd, 1H), 3.98 (dd, 1H), 5.72 (m, 1H), 7.60-7.79 (m, 3H), 7.90 (m, 1H), 8.62 (s, 1H), 9.27 (m, 1H), 9.38 (s, 1H), 9.55 (m, 1H).

### Preparation 56

### Ethyl 4-chloro-2-(6-fluoroimidazo[1,2-a]pyridin-3-yl)pyrimidine-5-carboxylate

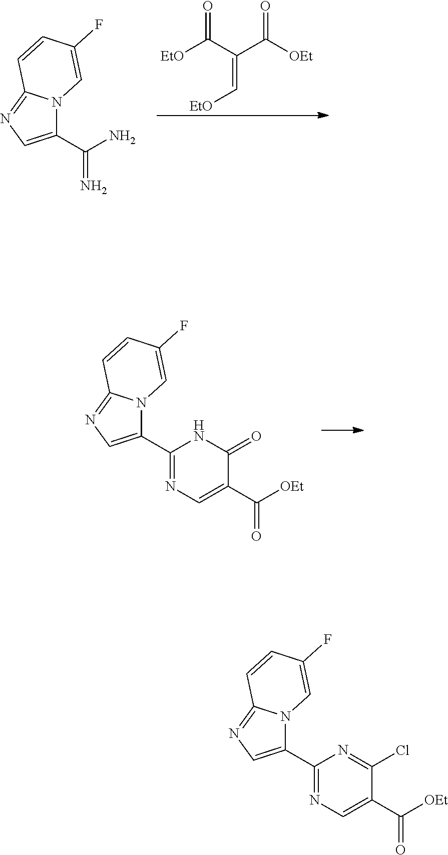 [SMILES: N=C(N)c1cnc2ccc(F)cn12 ; CCOC=C(C(=O)OCC)C(=O)OCC ; CCOC(=O)c1cnc(-c2cnc3ccc(F)cn23)[nH]c1=O ; CCOC(=O)c1cnc(-c2cnc3ccc(F)cn23)nc1Cl]

### a) Ethyl 2-(6-fluoroimidazo[1,2-a]pyridin-3-yl)-6-oxo-1,6-dihydropyrimidine-5-carboxylate

6-Fluoroimidazo[1,2-a]pyridine-3-carboximidamide hydrochloride (2.0 g, 9.3 mmol, Preparation 42b) and diethyl 2-(ethoxymethylene)malonate (2.0 g, 9.3 mmol) were added portionwise to a solution of sodium ethoxide in ethanol (21%, 8.14 mL, 18.6 mmol) and the resulting mixture was heated to reflux for 6 h. The precipitate formed was filtered, washed with ethanol and dried at 45° C. under vacuum in an oven to give 2.78 g (99%) of the title compound.

LRMS (m/z): 303 (M+1)⁺.

¹H-NMR δ (DMSO-d₆): 1.28 (t, 3H), 4.18 (q, 2H), 7.49 (t, 1H), 7.77 (dd, 1H), 8.36 (s, 1H), 8.57 (s, 1H), 10.22-10.36 (m, 1H).

### b) Ethyl 4-chloro-2-(6-fluoroimidazo[1,2-a]pyridin-3-yl)pyrimidine-5-carboxylate

A suspension of ethyl 2-(6-fluoroimidazo[1,2-a]pyridin-3-yl)-4-hydroxypyrimidine-5-carboxylate (310 mg, 1.03 mmol, Preparation 56a) in phosphorous oxychloride (2.1 mL, 23 mmol) was heated at 110° C. for 2 h. The excess of phosphorous oxychloride was evaporated under vacuum and the residue was poured onto ice. The resulting mixture was neutralised with 32% aqueous sodium hydroxide solution and extracted twice with ethyl acetate. The combined organic phase was washed with water and brine, dried over magnesium sulphate, filtered and the solvent eliminated under reduced pressure to yield 0.32 g (97%) of the title compound as a solid.

LRMS (m/z): 321 (M+1)⁺.

### Preparation 57

### (S)-Ethyl 2-(6-fluoroimidazo[1,2-a]pyridin-3-yl)-4-(1-(5-fluoropyridin-2-yl)ethylamino)pyrimidine-5-carboxylate

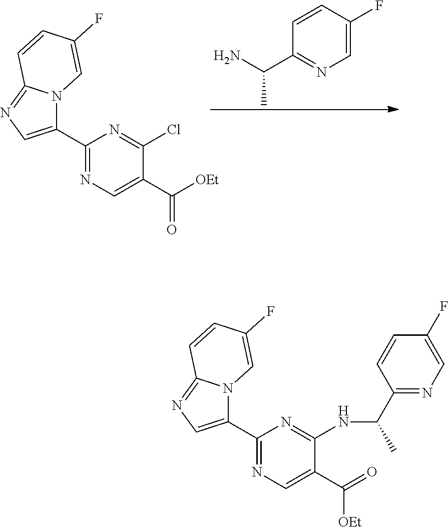

A mixture of ethyl 4-chloro-2-(6-fluoroimidazo[1,2-a]pyridin-3-yl)pyrimidine-5-carboxylate (1.3 g, 4.03 mmol, Preparation 56b), (S)-1-(5-fluoropyridin-2-yl)ethanamine hydrochloride (0.72 g, 4.08 mmol, prepared as described in WO2006/82392) and diisopropylethylamine (2.12 mL, 12.14 mmol) in THF (50 mL) was stirred overnight at room temperature. The solvent was evaporated under reduced pressure and the residue was partitioned between 4% aqueous sodium hydrogencarbonate solution and ethyl acetate. The organic layer was separated, washed with water, 4% aqueous hydrogen carbonate solution and brine, dried over sodium sulphate and the solvent removed under reduced pressure. The residue was purified by reverse phase chromatography (C-18 silica from Waters, water/1:1 acetonitrile-methanol as eluents [0.1% v/v formic acid buffered] 0% to 100%) to yield the title compound (0.63 g, 63%).

LRMS (m/z): 425 (M+1)⁺.

¹H-NMR δ (DMSO-d₆): 1.4 (t, 3H), 1.7 (d, 3H), 4.4 (q, 2H), 5.5 (t, 1H), 7.3 (m, 1H), 7.4 (m, 2H), 7.7 (dd, 1H), 8.5 (s, 1H), 8.6 (s, 1H), 8.9 (s, 1H), 9.1 (d, 1H), 9.7 (dd, 1H).

### Preparation 58

### Ethyl 2-(6-fluoroimidazo[1,2-a]pyridin-3-yl)-4-(pyridin-3-ylmethylamino) pyrimidine-5-carboxylate

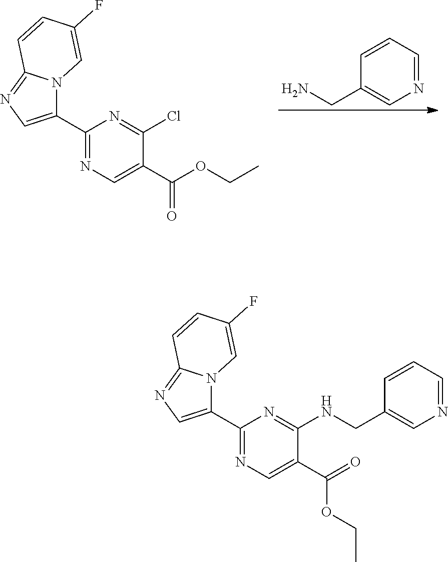 [SMILES: CCOC(=O)c1cnc(-c2cnc3ccc(F)cn23)nc1Cl ; NCc1cccnc1 ; CCOC(=O)c1cnc(-c2cnc3ccc(F)cn23)nc1NCc1cccnc1]

Obtained as a solid (71%) from ethyl 4-chloro-2-(6-fluoroimidazo[1,2-a]pyridin-3-yl)pyrimidine-5-carboxylate (600 mg, 1.87 mmol, Preparation 56b) and pyridin-3-ylmethanamine (190 μL, 1.87 mmol) following the experimental procedure as described in Preparation 57.

LRMS (m/z): 393 (M+1)⁺.

### Preparation 59

### Ethyl 2-(6-fluoroimidazo[1,2-a]pyridin-3-yl)-44(5-fluoropyridin-2-yl)methylamino) pyrimidine-5-carboxylate

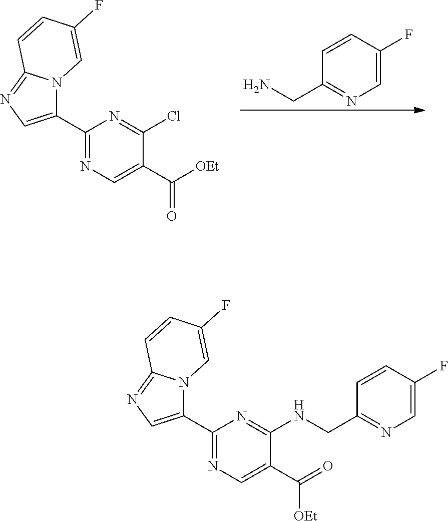 [SMILES: CCOC(=O)c1cnc(-c2cnc3ccc(F)cn23)nc1Cl ; NCc1ccc(F)cn1 ; CCOC(=O)c1cnc(-c2cnc3ccc(F)cn23)nc1NCc1ccc(F)cn1]

Obtained as a solid (62%) from ethyl 4-chloro-2-(6-fluoroimidazo[1,2-a]pyridin-3-yl)pyrimidine-5-carboxylate (510 mg, 1.59 mmol, Preparation 56b) and (5-fluoropyridin-2-yl)methanamine (200 mg, 1.59 mmol) following the experimental procedure as described in Preparation 57.

LRMS (m/z): 411 (M+1)⁺.

### Preparation 60

### (S)-Ethyl 2-(6-fluoroimidazo[1,2-a]pyridin-3-yl)-4-(1-(4-fluorophenyl)butylamino) pyrimidine-5-carboxylate

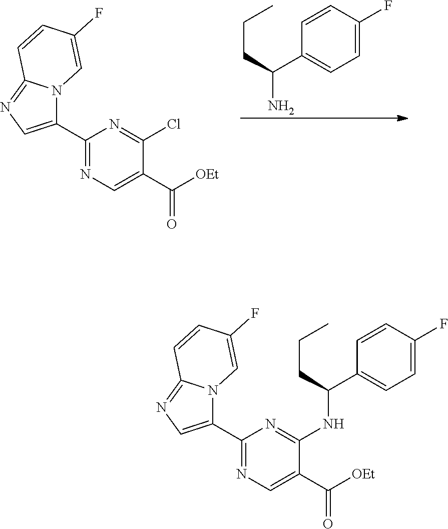 [SMILES: CCOC(=O)c1cnc(-c2cnc3ccc(F)cn23)nc1Cl ; CCC[C@H](N)c1ccc(F)cc1 ; CCC[C@H](Nc1nc(-c2cnc3ccc(F)cn23)ncc1C(=O)OCC)c1ccc(F)cc1]

Obtained as a solid (92%) from ethyl 4-chloro-2-(6-fluoroimidazo[1,2-a]pyridin-3-yl)pyrimidine-5-carboxylate (315 mg, 0.98 mmol, Preparation 56b) and (S)-1-(4-fluorophenyl)butan-1-amine hydrochloride (200 mg, 0.98 mmol) following the experimental procedure as described in Preparation 57.

LRMS (m/z): 452 (M+1)⁺.

### Preparation 61

### Ethyl 4-((1r,4r)-4-(cyanomethyl)cyclohexylamino)-2-(6-fluoroimidazo[1,2-a]pyridin-3-yl)pyrimidine-5-carboxylate

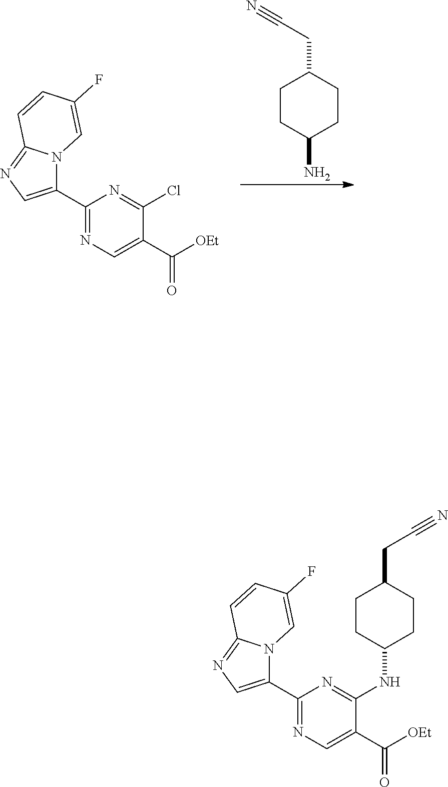

Obtained as a solid (66%) from ethyl 4-chloro-2-(6-fluoroimidazo[1,2-a]pyridin-3-yl)pyrimidine-5-carboxylate (93 mg, 0.29 mmol, Preparation 56b) and 2-((1r,4r)-4-aminocyclohexyl)acetonitrile (51 mg, 0.29 mmol, Preparation 47b) following the experimental procedure as described in Preparation 57.

LRMS (m/z): 423 (M+1)⁺.

### Preparation 62

### (R)-3-(3-(2-(6-Fluoroimidazo[1,2-a]pyridin-3-yl)-5-nitropyrimidin-4-ylamino) piperidin-1-yl)-3-oxopropanenitrile

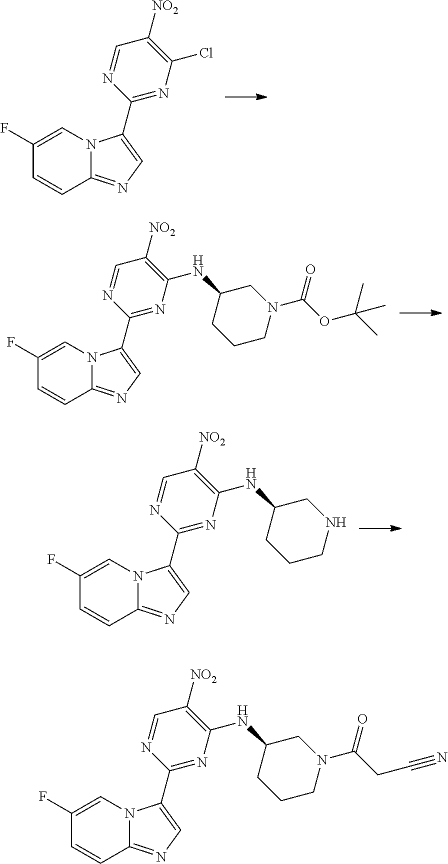 [SMILES: O=[N+]([O-])c1cnc(-c2cnc3ccc(F)cn23)nc1Cl ; CC(C)(C)OC(=O)N1CCC[C@@H](Nc2nc(-c3cnc4ccc(F)cn34)ncc2[N+](=O)[O-])C1 ; O=[N+]([O-])c1cnc(-c2cnc3ccc(F)cn23)nc1N[C@@H]1CCCNC1 ; N#CCC(=O)N1CCC[C@@H](Nc2nc(-c3cnc4ccc(F)cn34)ncc2[N+](=O)[O-])C1]

### a) (R)-tert-Butyl 3-(2-(6-fluoroimidazo[1,2-a]pyridin-3-yl)-5-nitropyrimidin-4-ylamino)piperidine-1-carboxylate

A mixture of 3-(4-chloro-5-nitropyrimidin-2-yl)-6-fluoroimidazo[1,2-a]pyridine (110 mg, 0.37 mmol, Preparation 49b), (R)-tert-butyl 3-aminopiperidine-1-carboxylate (83 mg, 0.41 mmol) and diisopropylethylamine (130 μL, 0.75 mmol) in tetrahydrofuran (1 mL) was stirred at room temperature overnight. The solvent was evaporated and the residue was partitioned between chloroform and 4% aqueous sodium hydrogencarbonate solution. The organic layer was separated, washed with brine, dried over magnesium sulphate and the solvent evaporated to yield 171 mg (100%) of the title compound as a yellow solid which was used in the next step without further purification.

LRMS (m/z): 458 (M+1)⁺

### b) (R)-2-(6-Fluoroimidazo[1,2-a]pyridin-3-yl)-5-nitro-N-(piperidin-3-yl)pyrimidin-4-amine

Trifluoroacetic acid (775 μL, 10.1 mmol) was added to a solution of (R)-tert-butyl 3-(2-(6-fluoroimidazo[1,2-a]pyridin-3-yl)-5-nitropyrimidin-4-ylamino)piperidine-1-carboxylate (230 mg, 0.50 mmol, Preparation 62a) in methylene chloride (2 mL) and the resulting mixture was stirred at room temperature until the reaction was completed. The solvents were evaporated and the residue was partitioned between methylene chloride and 2N aqueous sodium hydroxide solution. The organic layer was separated, washed with water and brine, dried over magnesium sulphate and the solvent evaporated to furnish 140 mg (78%) of the title compound as a yellow solid.

LRMS (m/z): 358 (M+1)⁺

### c) (R)-3-(3-(2-(6-Fluoroimidazo[1,2-a]pyridin-3-yl)-5-nitropyrimidin-4-ylamino) piperidin-1-yl)-3-oxopropanenitrile

Obtained as a yellow solid (32%) from (R)-2-(6-fluoroimidazo[1,2-a]pyridin-3-yl)-5-nitro-N-(piperidin-3-yl)pyrimidin-4-amine (140 mg, 0.39 mmol, Preparation 62b) and 3-[(2,5-dioxopyrrolidin-1-yl)oxy]-3-oxopropanenitrile (86 mg, 0.47 mmol, prepared as described in BE875054(A1)) following the experimental procedure as described in Preparation 34c. The crude product was purified by reverse phase chromatography (C-18 silica from Waters, water/1:1 acetonitrile-methanol as eluents [0.1% v/v formic acid buffered] 0% to 100%).

LRMS (m/z): 425 (M+1)⁺.

¹H NMR [symbol] (CDCl₃): 1.7-2.3 (m, 5H), 3.1-4.0 (m, 5H), 4.3-4.6 (m, 2H), 7.4 (m, 1H), 7.8 (m, 1H), 8.5 (m, 1H), 8.7 (m, 1H) 9.8 (m, 1H).

### Preparation 63

### 5-Cyclopropyl-2-(6-fluoroimidazo[1,2-a]pyridin-3-yl)pyrimidin-4-ol

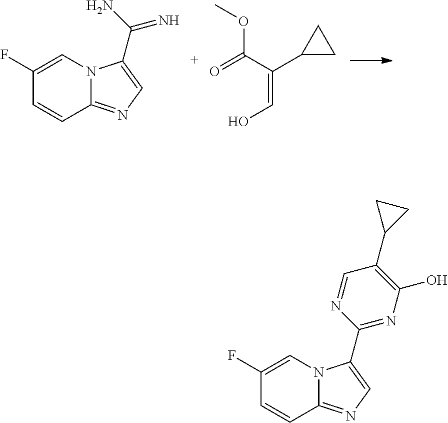

Butyllithium (1.62 mL of a 2.5 M solution in tetrahydrofuran, 4.05 mmol) was added to a solution of diisopropylamine (520 μL, 3.7 mmol) in anhydrous tetrahydrofuran (10 mL) at −20° C. under argon atmosphere. The resulting mixture was stirred at 0° C. for 45 minutes before being cooled to −78° C. and a solution of methyl 2-cyclopropylacetate (384 mg, 3.36 mmol, prepared as described in US2003/187040) in tetrahydrofuran (5 mL) was slowly added at this temperature. After 30 minutes at −78° C., a solution of ethyl formate (270 μL, 3.36 mmol) in tetrahydrofuran (5 mL) was added and the internal temperature was allowed to raise to −10° C. for 1 hour and then to room temperature for 1 additional hour. Finally, a solution of 6-fluoroimidazo[1,2-a]pyridine-3-carboximidamide (600 mg, 3.37 mmol, Preparation 42b) in tetrahydrofuran (10 mL) was added to the reaction mixture and the resulting solution was stirred overnight at room temperature before being heated under microwave irradiation at 140° C. for 2 hours. The solvent was removed under vacuum and the residue was dissolved in water. After neutralization to pH=4 by addition of 2N aqueous hydrochloric acid solution, the precipitate formed was filtered, washed with water and diethyl ether and dried to give 90 mg (10%) of the title compound as a white solid.

LRMS (m/z): 271 (M+1)⁺.

¹H NMR [symbol] (DMSO-d₆): 0.8 (m, 4H), 1.9 (s, 1H), 7.6 (s, 1H), 7.8 (s, 1H), 7.9 (s, 1H), 8.7 (s, 1H), 9.9 (s, 1H), 12.8 (s, 1H).

### Preparation 64

### (R)-5-Cyclopropyl-2-(6-fluoroimidazo[1,2-a]pyridin-3-yl)-N-(piperidin-3-yl)pyrimidin-4-amine

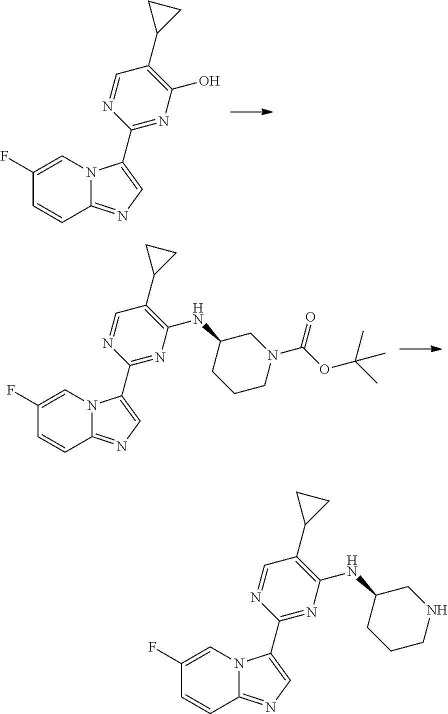

### a) (R)-tert-Butyl 3-(5-cyclopropyl-2-(6-fluoroimidazo[1,2-a]pyridin-3-yl)pyrimidin-4-ylamino)piperidine-1-carboxylate

Obtained as an oil from 5-cyclopropyl-2-(6-fluoroimidazo[1,2-a]pyridin-3-yl)pyrimidin-4-ol (85 mg, 0.31 mmol, Preparation 63) and (R)-tert-butyl 3-aminopiperidine-1-carboxylate (188 mg, 0.94 mmol) following the experimental procedure as described in Preparation 5a. The crude product was used in the next step without further purification.

LRMS (m/z): 453 (M+1)⁺.

### b) (R)-5-Cyclopropyl-2-(6-fluoroimidazo[1,2-a]pyridin-3-yl)-N-(piperidin-3-yl)pyrimidin-4-amine

Trifluoroacetic acid (121 μL, 1.57 mmol) was added to a solution of (R)-tert-butyl 3-(5-cyclopropyl-2-(6-fluoroimidazo[1,2-a]pyridin-3-yl)pyrimidin-4-ylamino)piperidine-1-carboxylate (Preparation 64a) in methylene chloride (2 mL) and the reaction mixture was stirred at room temperature overnight. The solvent was evaporated and the residue was dissolved in water, basified with a saturated aqueous solution of potassium carbonate and extracted with chloroform. The organic phase was washed with brine, dried over magnesium sulphate, filtered and the solvent evaporated to yield 95 mg (86% in two steps) of the title compound as a yellowish solid.

LRMS (m/z): 353 (M+1)⁺.

¹H NMR [symbol] (CDCl₃): 0.6 (m, 2H), 1.0 (d, 2H), 1.5-2.0 (m, 6H), 2.8 (m, 3H), 3.2 (dd, 1H), 4.3 (m, 1H), 5.8 (d, 1H), 7.2 (m, 1H), 7.6 (dd, 1H), 8.0 (s, 1H), 8.5 (s, 1H) 10.0 (dd, 1H).

### Preparation 65

### 4-Chloro-2-(6-fluoroimidazo[1,2-a]pyridin-3-yl)pyrimidine-5-carbonitrile

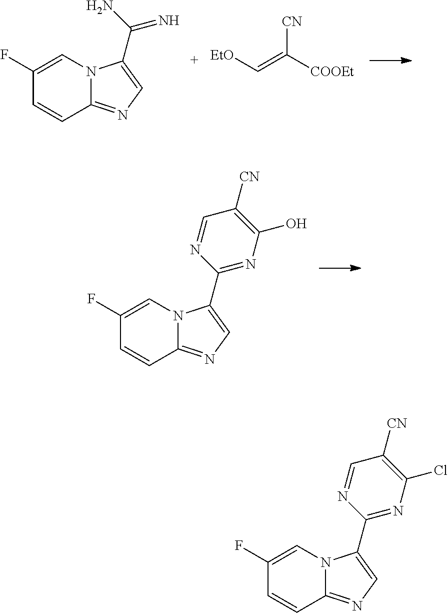 [SMILES: N=C(N)c1cnc2ccc(F)cn12 ; CCO/C=C(\C#N)C(=O)OCC ; N#Cc1cnc(-c2cnc3ccc(F)cn23)nc1O ; N#Cc1cnc(-c2cnc3ccc(F)cn23)nc1Cl]

### a) 2-(6-Fluoroimidazo[1,2-a]pyridin-3-yl)-4-hydroxypyrimidine-5-carbonitrile

Triethylamine (2.89 mL, 20.7 mmol) was added to a suspension of 6-fluoroimidazo[1,2-a]pyridine-3-carboximidamide (1.00 g, 4.66 mmol, Preparation 42b) and (Z)-ethyl 2-cyano-3-ethoxyacrylate (1.23 g, 7.27 mmol) in ethanol (15 mL) and the resulting mixture was stirred at 90° C. in a sealed tube under argon for 3 hours. The solvent was then evaporated and the residue was dissolved in chloroform. The organic solution was washed with water and brine, dried over sodium sulphate and the solvent was evaporated. The crude product was purified by flash chromatography (dichloromethane to 9:1 dichloromethane/methanol) to yield 0.59 g (50%) of the title compound as a brown solid.

LRMS (m/z): 256 (M+1)⁺.

¹H NMR [symbol] (CDCl₃): 7.5 (m, 1H), 7.8 (dd, 1H), 8.2 (s, 1H), 8.3 (s, 1H), 10.2 (dd, 1H).

### b) 4-Chloro-2-(6-fluoroimidazo[1,2-a]pyridin-3-yl)pyrimidine-5-carbonitrile

A suspension of 2-(6-Fluoroimidazo[1,2-a]pyridin-3-yl)-4-hydroxypyrimidine-5-carbonitrile (50 mg, 0.20 mmol, Preparation 65a) in phosphorus oxychloride (0.5 mL) was stirred at 110° C. for 90 minutes and then it was poured into a mixture of water and ice. The aqueous solution was basified with an aqueous solution of sodium hydroxide and it was extracted with ethyl acetate. The organic layer was washed with brine, dried over sodium sulphate and the solvent was evaporated to give 50 mg (93%) of the title compound.

LRMS (m/z): 438 (M−1-1)⁺.

¹H NMR [symbol] (CDCl₃): 7.4 (m, 1H), 7.8 (dd-1H), 8.8 (s, 1H), 8.9 (s, 1H), 9.8 (dd, 1H).

### Preparation 66

### (R)-2-(6-Fluoroimidazo[1,2-a]pyridin-3-yl)-4-(piperidin-3-ylamino)pyrimidine-5-carbonitrile

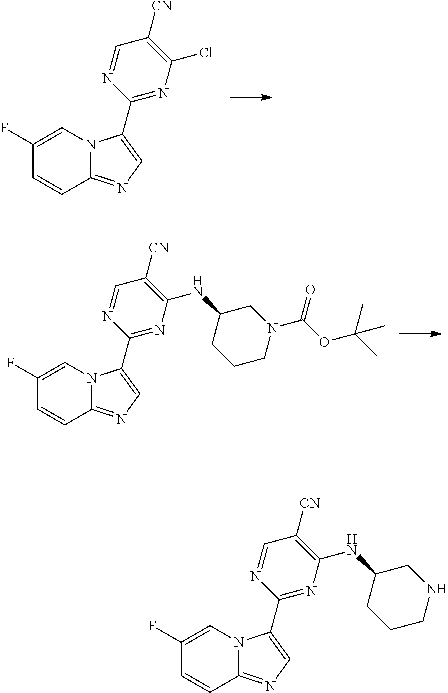 [SMILES: N#Cc1cnc(-c2cnc3ccc(F)cn23)nc1Cl ; CC(C)(C)OC(=O)N1CCC[C@@H](Nc2nc(-c3cnc4ccc(F)cn34)ncc2C#N)C1 ; N#Cc1cnc(-c2cnc3ccc(F)cn23)nc1N[C@@H]1CCCNC1]

### a) (R)-tert-Butyl 3-(5-cyano-2-(6-fluoroimidazo[1,2-a]pyridin-3-yl)pyrimidin-4-ylamino)piperidine-1-carboxylate

(R)-Tert-butyl 3-aminopiperidine-1-carboxylate (40 mg, 0.20 mmol) and diisopropylethylamine (65 μL, 0.37 mmol) were added to a solution of 4-chloro-2-(6-fluoroimidazo[1,2-a]pyridin-3-yl)pyrimidine-5-carbonitrile (50 mg, 0.18 mmol, Preparation 65b) in tetrahydrofuran (2 mL) and the resulting mixture was stirred at room temperature for 6 hours. The solvent was then evaporated and the residue was partitioned between methylene chloride and 4% aqueous sodium bicarbonate solution. The organic layer was separated, washed with brine, dried over magnesium sulphate and the solvent was evaporated to yield 80 mg (90%) of the title product.

LRMS (m/z): 438 (M+1)⁺.

### b) (R)-2-(6-Fluoroimidazo[1,2-a]pyridin-3-yl)-4-(piperidin-3-ylamino)pyrimidine-5-carbonitrile

Obtained as a solid (54%) from (R)-tert-butyl 3-(5-cyano-2-(6-fluoroimidazo[1,2-a]pyridin-3-yl)pyrimidin-4-ylamino)piperidine-1-carboxylate (80 mg, 0.16 mmol, Preparation 66a) following the experimental procedure described in Preparation 5b. The crude product obtained was used in the next step without further purification.

LRMS (m/z): 338 (M+1)⁺.

### Preparation 67

### (R)-2-(6-Fluoroimidazo[1,2-a]pyridin-3-yl)-4-(piperidin-3-ylamino)pyrimidine-5-carbonitrile

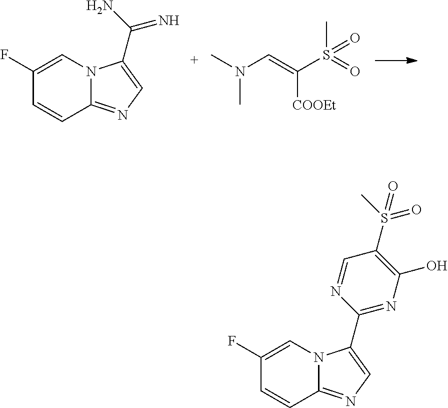 [SMILES: N=C(N)c1cnc2ccc(F)cn12 ; CCOC(=O)/C(=C\N(C)C)S(C)(=O)=O ; CS(=O)(=O)c1cnc(-c2cnc3ccc(F)cn23)nc1O]

Sodium carbonate (190 mg, 1.79 mmol) was added to a solution of 6-fluoroimidazo[1,2-a]pyridine-3-carboximidamide (330 mg, 1.49 mmol, Preparation 42b) and (E)-ethyl 3-(dimethylamino)-2-(methylsulfonyl)acrylate (768 mg, 3.58 mmol, prepared according to US2004/6743798) in methanol (15 mL) and water (0.5 mL) and the resulting mixture was heated to reflux overnight. The solvent was evaporated and the crude product was purified by flash chromatography (dichloromethane to 8:2 dichloromethane/methanol) to yield 250 mg (54%) of the title compound as a brown solid.

LRMS (m/z): 460 (M+1)⁺.

¹H NMR [symbol] (DMSO-d₆): 3.2 (s, 3H), 7.6 (m, 1H), 7.8 (dd, 1H), 8.4 (s, 1H), 8.6 (s, 1H), 10.1 (dd, 1H).

### Preparation 68

### (R)-2-(6-Fluoroimidazo[1,2-a]pyridin-3-yl)-5-(methylsulfonyl)-N-(piperidin-3-yl)pyrimidin-4-amine

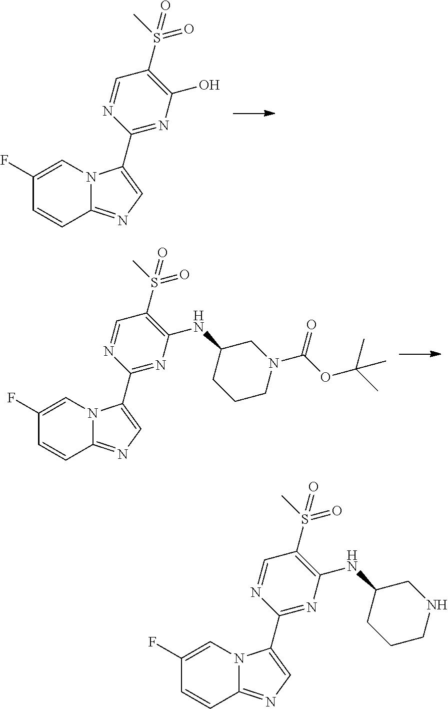

### a) (R)-tert-Butyl 3-(2-(6-fluoroimidazo[1,2-a]pyridin-3-yl)-5-(methylsulfonyl)pyrimidin-4-ylamino)piperidine-1-carboxylate

Obtained (29% yield) as a solid from (R)-2-(6-fluoroimidazo[1,2-a]pyridin-3-yl)-4-(piperidin-3-ylamino)pyrimidine-5-carbonitrile (220 mg, 0.71 mmol, Preparation 67) and (R)-tert-butyl 3-aminopiperidine-1-carboxylate (430 mg, 2.15 mmol) following the experimental procedure as described in Preparation 5a. The crude product was purified by flash chromatography (0 to 100% hexane/ethyl acetate and then 0 to 10% ethyl acetate/methanol).

LRMS (m/z): 491 (M+1)⁺.

### b) (R)-2-(6-Fluoroimidazo[1,2-a]pyridin-3-yl)-5-(methylsulfonyl)-N-(piperidin-3-yl)pyrimidin-4-amine

Obtained as a solid (38%) from (R)-tert-butyl 3-(2-(6-fluoroimidazo[1,2-a]pyridin-3-yl)-5-(methylsulfonyl)pyrimidin-4-ylamino)piperidine-1-carboxylate (100 mg, 0.20 mmol, Preparation 68a) following the experimental procedure described in Preparation 5b. The crude product was used in the next step without further purification.

LRMS (m/z): 391 (M+1)⁺.

### Preparation 69

### (R)-Ethyl 2-(6-fluoroimidazo[1,2-a]pyridin-3-yl)-4-(piperidin-3-ylamino)pyrimidine-5-carboxylate

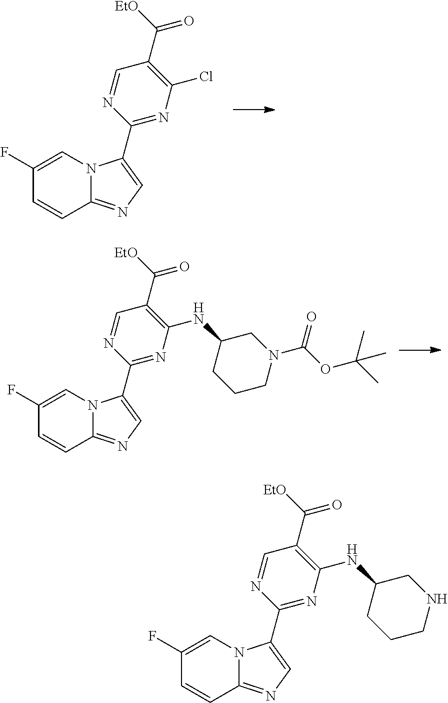

### a) (R)-Ethyl 4-(1-(tert-butoxycarbonyl)piperidin-3-ylamino)-2-(6-fluoroimidazo[1,2-a]pyridin-3-yl)pyrimidine-5-carboxylate

Obtained (67% yield) as a solid from ethyl 4-chloro-2-(6-fluoroimidazo[1,2-a]pyridin-3-yl)pyrimidine-5-carboxylate (830 mg, 2.59 mmol, Preparation 56b) and (R)-tert-butyl 3-aminopiperidine-1-carboxylate (570 mg, 2.85 mmol) following the experimental procedure as described in Preparation 66a. The crude product was purified by reverse phase chromatography (C-18 silica from Waters, water/1:1 acetonitrile-methanol as eluents [0.1% v/v formic acid buffered] 0% to 100%).

LRMS (m/z): 485 (M+1)⁺.

### b) (R)-Ethyl 2-(6-fluoroimidazo[1,2-a]pyridin-3-yl)-4-(piperidin-3-ylamino) pyrimidine-5-carboxylate

Obtained as a solid (93%) from (R)-ethyl 4-(1-(tert-butoxycarbonyl)piperidin-3-ylamino)-2-(6-fluoroimidazo[1,2-a]pyridin-3-yl)pyrimidine-5-carboxylate (840 mg, 1.73 mmol, Preparation 69a) following the experimental procedure described in Preparation 5b. The crude product was used in the next step without further purification.

LRMS (m/z): 385 (M+1)⁺.

¹H NMR [symbol] (CDCl₃): 1.4 (t, 3H), 1.6-2.1 (m, 4H), 2.0 (d, 1H), 2.8-2.9 (m, 3H), 3.3 (dd, 1H), 4.3 (m, 1H), 4.4 (q, 2H), 7.3 (m, 1H), 7.7 (dd, 1H), 8.6 (m, 2H), 8.9 (s, 1H), 10.0 (dd, 1H).

### Preparation 70

### 3-(4-Chloro-5-methoxypyrimidin-2-yl)-6-fluoroimidazo[1,2-a]pyridine

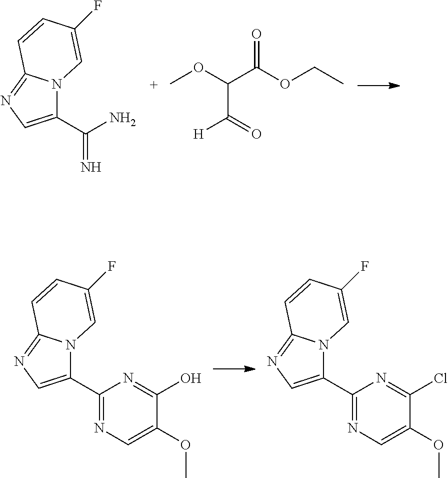

### a) 2-(6-Fluoroimidazo[1,2-a]pyridin-3-yl)-5-methoxypyrimidin-4-ol

Ethyl formate (570 μL, 7.08 mmol) and ethyl 2-methoxyacetate (530 μL, 4.52 mmol) were added to a suspension of sodium hydride (120 mg of a 60% suspension in mineral oil, 5.00 mmol) in tetrahydrofuran (2 mL) and the resulting mixture was stirred at room temperature overnight. Then a sodium methoxide solution in methanol (25%, 1.30 mL) and a solution 6-fluoroimidazo[1,2-a]pyridine-3-carboximidamide (1.0 g, 4.66 mmol, Preparation 42b) in isopropanol (2 mL) were added. The resulting mixture was heated to reflux for 2 h before the solvents were removed in vacuo. The residue was dissolved in water and acetic acid was added until a precipitate formed. The white solid was filtered, washed with water and dried to give 460 mg (39%) of the title compound.

LRMS (m/z): 261 (M+1)⁺

¹H NMR [symbol] (DMSO-d₆): 3.8 (m, 3H), 7.6 (m, 1H), 7.7 (s, 1H), 7.8 (m, 1H), 8.6 (s, 1H), 9.8 (m, 1H), 12.6 (bs, 1H).

### b) 3-(4-Chloro-5-methoxypyrimidin-2-yl)-6-fluoroimidazo[1,2-a]pyridine

410 mg (61%) of the title compound were obtained from 2-(6-fluoroimidazo[1,2-a]pyridin-3-yl)-5-methoxypyrimidin-4-ol (630 mg, 2.42 mmol, Preparation 70a) following the experimental procedure described in Preparation 65b.

LRMS (m/z): 279 (M+1)⁺.

### Preparation 71

### (R)-2-(6-Fluoroimidazo[1,2-a]pyridin-3-yl)-5-methoxy-N-(piperidin-3-yl)pyrimidin-4-amine

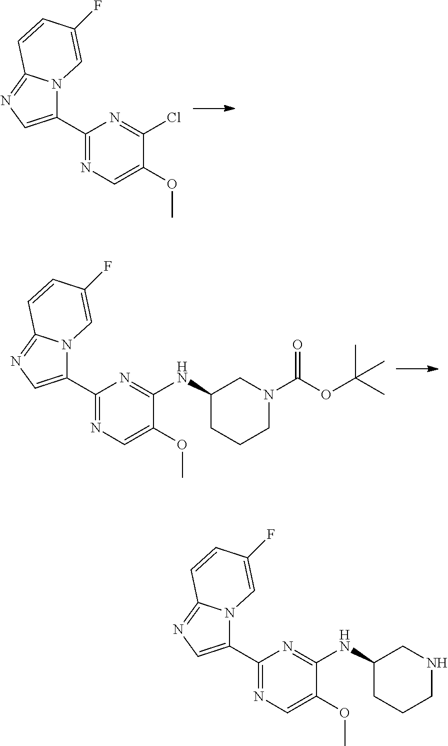 [SMILES: COc1cnc(-c2cnc3ccc(F)cn23)nc1Cl ; COc1cnc(-c2cnc3ccc(F)cn23)nc1N[C@@H]1CCCN(C(=O)OC(C)(C)C)C1 ; COc1cnc(-c2cnc3ccc(F)cn23)nc1N[C@@H]1CCCNC1]

### a) (R)-tert-Butyl 3-(2-(6-fluoroimidazo[1,2-a]pyridin-3-yl)-5-methoxypyrimidin-4-ylamino)piperidine-1-carboxylate

Obtained (41% yield) as a solid from 3-(4-chloro-5-methoxypyrimidin-2-yl)-6-fluoroimidazo[1,2-a]pyridine (410 mg, 1.47 mmol, Preparation 70b) and (R)-tert-butyl 3-aminopiperidine-1-carboxylate (295 mg, 1.47 mmol) following the experimental procedure as described in Preparation 66a. The reaction mixture was stirred at reflux overnight. The crude product was purified by reverse phase chromatography (C-18 silica from Waters, water/1:1 acetonitrile-methanol as eluents [0.1% v/v formic acid buffered] 0% to 100%).

LRMS (m/z): 443 (M+1)⁺

¹H NMR [symbol] (CDCl₃): 1.4 (s, 9H), 1.6-2.0 (m, 4H), 3.4-3.7 (m, 4H), 3.9 (s, 3H), 4.2 (d, 1H), 5.5 (bs, 1H), 7.2 (m, 1H), 7.7 (dd, 1H), 7.8 (s, 1H), 8.4 (s, 1H), 9.9 (dd, 1H).

### b) (R)-2-(6-Fluoroimidazo[1,2-a]pyridin-3-yl)-5-methoxy-N-(piperidin-3-yl)pyrimidin-4-amine

Obtained as a solid (91%) from (R)-tert-butyl 3-(2-(6-fluoroimidazo[1,2-a]pyridin-3-yl)-5-methoxypyrimidin-4-ylamino)piperidine-1-carboxylate (300 mg, 0.68 mmol, Preparation 71a) following the experimental procedure described in Preparation 5b. The crude product was used in the next step without further purification.

LRMS (m/z): 343 (M+1)⁺.

### Preparation 72

### 3-(4,6-Dichloropyrimidin-2-yl)-6-fluoroimidazo[1,2-a]pyridine

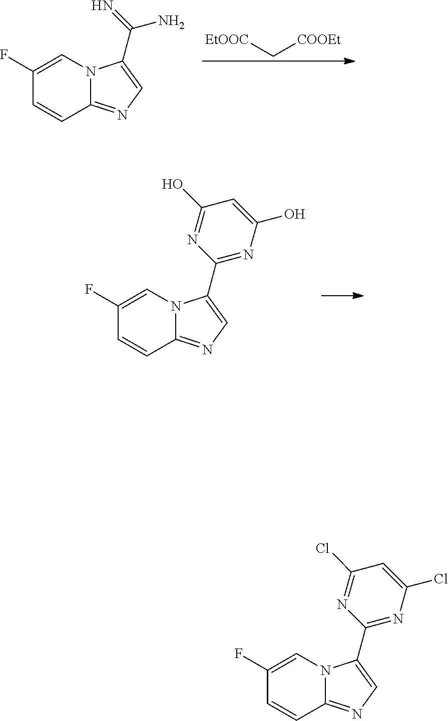 [SMILES: N=C(N)c1cnc2ccc(F)cn12 ; CCOC(=O)CC(=O)OCC ; Oc1cc(O)nc(-c2cnc3ccc(F)cn23)n1 ; Fc1ccc2ncc(-c3nc(Cl)cc(Cl)n3)n2c1]

### a) 2-(6-Fluoroimidazo[1,2-a]pyridin-3-yl)pyrimidine-4,6-diol

6-Fluoroimidazo[1,2-a]pyridine-3-carboximidamide (7.0 g, 32.6 mmol, Preparation 42b) and diethyl malonate (9.9 mL, 65.2 mmol) were added to a solution of sodium (2.25 g, 97.8 mmol) in methanol (140 mL) at 0° C. and the resulting mixture was stirred at room temperature for 40 hours. The solvent was evaporated and the residue was dissolved in 200 mL of water. The resulting solution was stirred at room temperature for 1 hour and then acidified with HCl 5N until a white solid precipitated. The product was filtered, washed with water and diethyl ether and dried to yield the title compound (53%), which was used in the next step without further purification.

LRMS (m/z): 247 (M+1)⁺

¹H NMR [symbol] (DMSO-d₆): 5.3 (bs, 1H), 7.6 (m, 1H), 7.9 (dd, 1H), 8.8 (bs, 1H), 10.1 (dd, 1H), 11.5 (bs, 1H), 12.4 (bs, 1H).

### b) 3-(4,6-Dichloropyrimidin-2-yl)-6-fluoroimidazo[1,2-a]pyridine

A suspension of 2-(6-fluoroimidazo[1,2-a]pyridin-3-yl)pyrimidine-4,6-diol (4.28 g, 17.38 mmol, Preparation 72a) in phosphorus oxychloride (30 mL) was refluxed with stirring overnight. The excess of phosphorus oxychloride was evaporated and the resulting crude was partitioned between water and methylene chloride. The organic phase was washed with water and brine, dried over sodium sulphate and the solvent was removed under reduced pressure to give the title compound (67%) as a solid.

LRMS (m/z): 283 (M+1)⁺

¹H NMR [symbol] (CDCl₃): 7.2 (s, 1H), 7.4 (m, 1H), 7.8 (dd, 1H), 8.7 (s, 1H), 9.8 (dd, 1H).

### Preparation 73

### (R)-tert-Butyl 3-(6-chloro-2-(6-fluoroimidazo[1,2-a]pyridin-3-yl)pyrimidin-4-ylamino)piperidine-1-carboxylate

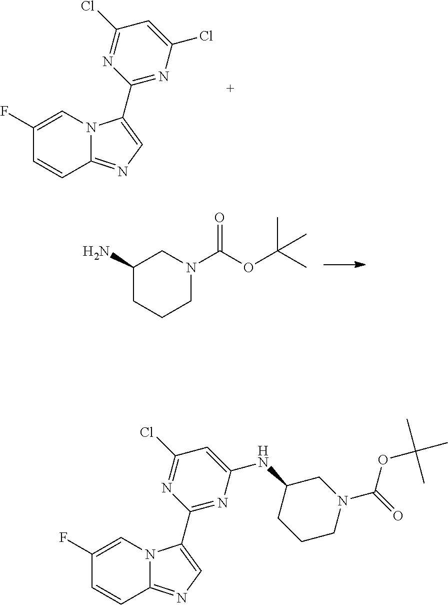 [SMILES: Fc1ccc2ncc(-c3nc(Cl)cc(Cl)n3)n2c1 ; CC(C)(C)OC(=O)N1CCC[C@@H](N)C1 ; CC(C)(C)OC(=O)N1CCC[C@@H](Nc2cc(Cl)nc(-c3cnc4ccc(F)cn34)n2)C1]

(R)-Tert-butyl 3-aminopiperidine-1-carboxylate (1.41 g, 7.04 mmol) was added to a suspension of 3-(4,6-dichloropyrimidin-2-yl)-6-fluoroimidazo[1,2-a]pyridine (1.0 g, 3.53 mmol, Preparation 72b) in ethanol (60 mL) and the resulting mixture was heated to reflux overnight. The solvent was evaporated and the crude obtained was partitioned between water and chloroform. The organic phase was separated, washed with water and brine, dried over magnesium sulphate and the solvent was evaporated. The residue was purified by flash chromatography (dichloromethane to 94:6 dichloromethane/methanol) to yield 1.38 g (87%) of the title product as a solid.

LRMS (m/z): 447 (M+1)⁺

¹H NMR [symbol] (CDCl₃): 1.5 (s, 9H), 1.5-2.2 (m, 5H), 3.3-3.8 (m, 4H), 5.2 (s, 1H), 6.2 (s, 1H), 7.3 (m, 1H), 7.7 (dd, 1H), 8.6 (s, 1H), 9.8 (dd, 1H).

### Preparation 74

### (R)-2-(6-Fluoroimidazo[1,2-a]pyridin-3-yl)-N-(piperidin-3-yl)-6-(1H-1,2,4-triazol-1-yl)pyrimidin-4-amine

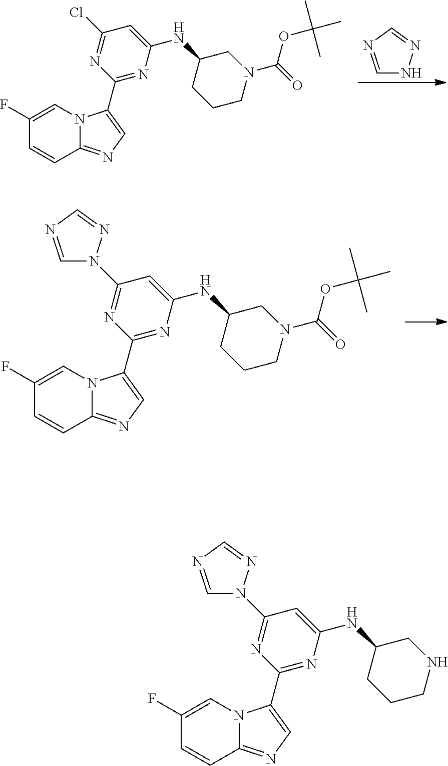

### a) (R)-tert-Butyl 3-(2-(6-fluoroimidazo[1,2-a]pyridin-3-yl)-6-(1H-1,2,4-triazol-1-yl)pyrimidin-4-ylamino)piperidine-1-carboxylate

1H-1,2,4-Triazole (77 mg, 1.11 mmol) and cesium carbonate (364 mg, 1.12 mmol) were added to a solution of (R)-tert-butyl 3-(6-chloro-2-(6-fluoroimidazo[1,2-a]pyridin-3-yl)pyrimidin-4-ylamino)piperidine-1-carboxylate (100 mg, 0.22 mmol, Preparation 73) in N,N′-dimethylformamide (3 mL) and the resulting mixture was heated at 130° C. for 1 hour and then left overnight at room temperature. The solvent was evaporated and the residue was treated with water. The white solid precipitated was filtered, washed with water and dried to yield 99 mg (92%) of the title compound.

LRMS (m/z): 480 (M+1)⁺

¹H NMR [symbol] (CDCl₃): 1.5 (s, 9H), 1.6-2.1 (m, 4H), 3.4-3.9 (m, 5H), 6.7 (s, 1H), 7.3 (m, 1H), 7.7 (m, 1H), 8.1 (s, 1H), 8.6 (s, 1H), 9.2 (s, 1H), 9.8 (s, 1H).

### b) (R)-3-(2-(6-Fluoroimidazo[1,2-a]pyridin-3-yl)-6-(1H-1,2,4-triazol-1-yl)pyrimidin-4-ylamino)piperidine

Trifluoroacetic acid (80 μL) was added to a solution of (R)-tert-butyl 3-(2-(6-fluoroimidazo[1,2-a]pyridin-3-yl)-6-(1H-1,2,4-triazol-1-yl)pyrimidin-4-ylamino)piperidine-1-carboxylate (99 mg, 0.21 mmol, Preparation 74a) in methylene chloride (2 mL) and the resulting mixture was left at room temperature overnight. Additional trifluoroacetic acid (80 μL) was then added and once the reaction was completed the reaction mixture was diluted with methylene chloride and water. The aqueous phase was separated, basified by slow addition of a saturated aqueous solution of potassium carbonate and extracted several times with chloroform. The combined organic phases were washed with water and brine, dried over magnesium sulphate and the solvent evaporated to yield 61 mg (78%) of the title product as a white solid.

LRMS (m/z): 380 (M+1)⁺

¹H NMR [symbol] (CDCl₃): 1.6-2.0 (m, 5H), 2.8-3.2 (m, 4H), 5.9 (s, 1H), 6.7 (s, 1H), 7.3 (m, 1H), 7.7 (dd, 1H), 8.1 (s, 1H), 8.6 (s, 1H), 9.2 (s, 1H), 9.8 (dd, 1H).

### Preparation 75

### (R)-2-(6-Fluoroimidazo[1,2-a]pyridin-3-yl)-6-(4-methylpiperazin-1-yl)-N-(piperidin-3-yl)pyrimidin-4-amine

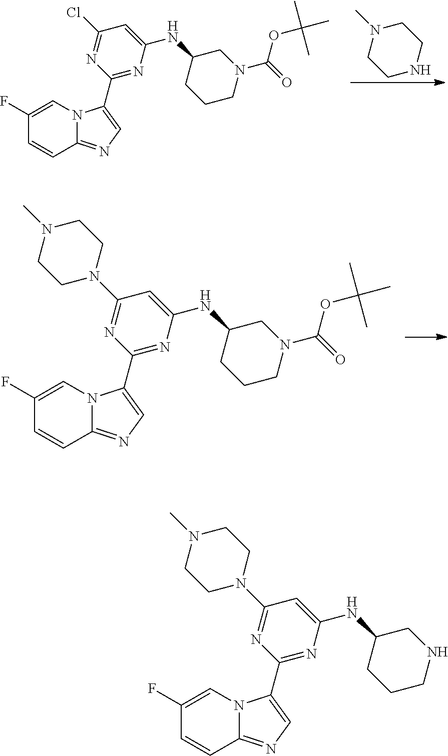

### a) (R)-tert-Butyl 3-(2-(6-fluoroimidazo[1,2-a]pyridin-3-yl)-6-(4-methylpiperazin-1-yl)pyrimidin-4-ylamino)piperidine-1-carboxylate

Prepared from (R)-tert-butyl 3-(6-chloro-2-(6-fluoroimidazo[1,2-a]pyridin-3-yl)pyrimidin-4-ylamino)piperidine-1-carboxylate (100 mg, 0.22 mmol, Preparation 73) and N-methylpiperazine (120 μL, 1.12 mmol) following the procedure described in Preparation 74. The product was purified by flash chromatography (dichloromethane to 9:1 dichloromethane/methanol) to give 68 mg (60%) of the title product as a solid.

LRMS (m/z): 511 (M+1)⁺

¹H NMR [symbol] (CDCl₃): 1.4 (s, 9H), 1.6-2.1 (m, 5H), 2.3 (s, 3H), 2.5 (m, 4H), 3.2-3.9 (m, 8H), 4.7 (d, 1H), 5.4 (s, 1H), 7.2 (m, 1H), 7.6 (dd, 1H), 8.4 (s, 1H), 9.8 (dd, 1H).

### b) (R)-2-(6-Fluoroimidazo[1,2-a]pyridin-3-yl)-6-(4-methylpiperazin-1-yl)-N-(piperidin-3-yl)pyrimidin-4-amine

Prepared from (R)-tert-butyl 3-(2-(6-fluoroimidazo[1,2-a]pyridin-3-yl)-6-(4-methylpiperazin-1-yl)pyrimidin-4-ylamino)piperidine-1-carboxylate (68 mg, 0.13 mmol, Preparation 75a) and trifluoroacetic acid (51 μL, 0.66 mmol) following the procedure described in Preparation 74b. The title compound was obtained as a white solid (55 mg, 100%) which was used in the next step without further purification.

LRMS (m/z): 411 (M+1)⁺

¹H NMR [symbol] (CDCl₃): 1.5-2.0 (m, 5H), 2.3 (s, 3H), 2.5 (m, 4H), 2.6-2.9 (m, 3H), 3.2 (dd, 1H), 3.6 (m, 4H), 3.8 (bs, 1H), 5.0 (d, 1H), 5.4 (s, 1H), 7.2 (m, 1H), 7.6 (dd, 1H), 8.4 (s, 1H), 9.9 (dd, 1H).

### Preparation 76

### (R)-Benzyl 4-(6-(1-(2-cyanoacetyl)piperidin-3-ylamino)-2-(6-fluoroimidazo[1,2-a]pyridin-3-yl)pyrimidin-4-yl)piperazine-1-carboxylate

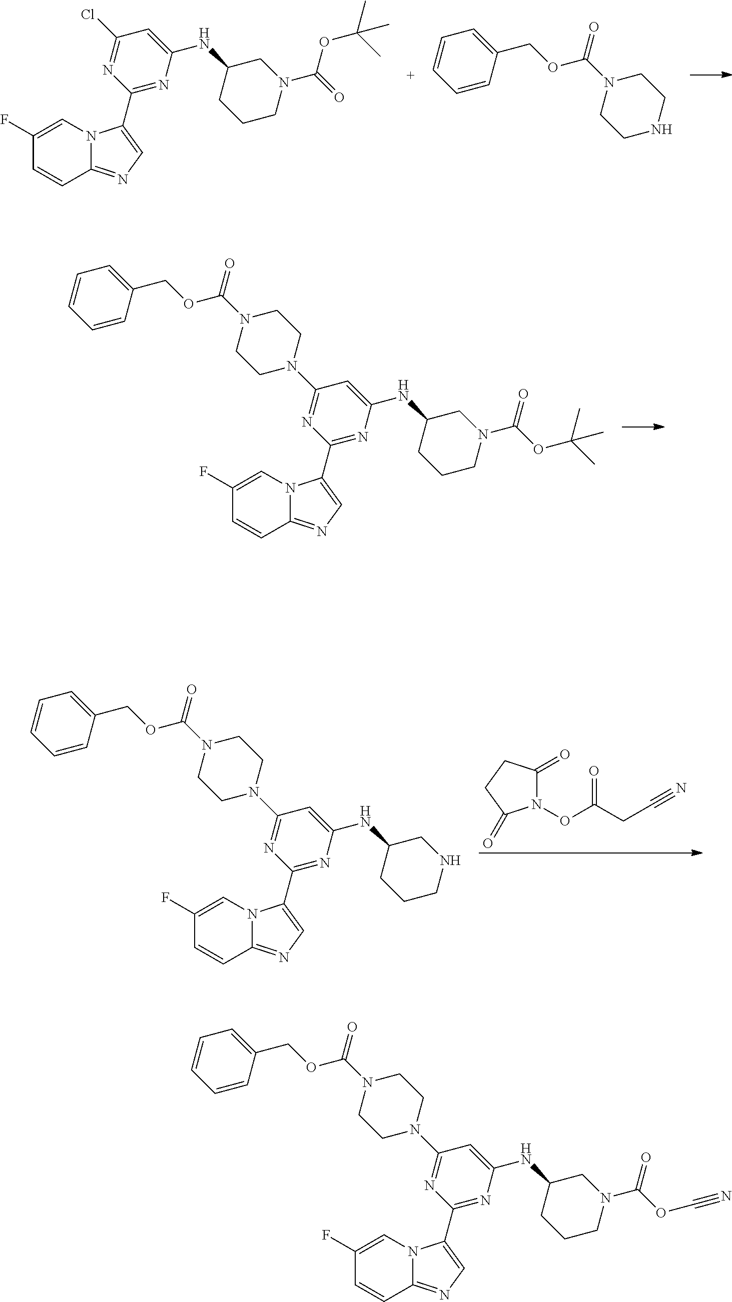

### a) (R)-Benzyl 4-(6-(1-(tert-butoxycarbonyl)piperidin-3-ylamino)-2-(6-fluoroimidazo[1,2-a]pyridin-3-yl)pyrimidin-4-yl)piperazine-1-carboxylate

Benzyl piperazine-1-carboxylate (540 μL, 2.79 mmol) was added to a solution of (R)-tert-butyl 3-(6-chloro-2-(6-fluoroimidazo[1,2-a]pyridin-3-yl)pyrimidin-4-ylamino) piperidine-1-carboxylate (250 mg, 0.56 mmol, Preparation 73) in N-methylpirrolidone (2 mL) and the resulting mixture was heated at 130° C. under microwave irradiation for 2 hours. The reaction mixture was partitioned between water and methylene chloride and the organic phase was separated, washed with water and brine, dried over magnesium sulphate and the solvent evaporated. The product was purified by flash chromatography (dichloromethane to 93:7 dichloromethane/methanol) to yield 339 mg (96%) of the title product as a light orange solid.

LRMS (m/z): 631 (M+1)⁺

¹H NMR [symbol] (CDCl₃): 1.4 (bs, 9H), 1.6-2.1 (m, 5H), 2.4 (m, 4H), 3.1-3.9 (m, 8H), 4.8 (bs, 1H), 5.1 (s, 2H), 5.4 (bs, 1H), 7.2 (m, 1H), 7.3 (m, 5H), 7.6 (dd, 1H), 8.4 (s, 1H), 9.8 (dd, 1H).

### b) (R)-Benzyl 4-(2-(6-fluoroimidazo[1,2-a]pyridin-3-yl)-6-(piperidin-3-ylamino) pyrimidin-4-yl)piperazine-1-carboxylate

Prepared from (R)-benzyl 4-(6-(1-(tert-butoxycarbonyl)piperidin-3-ylamino)-2-(6-fluoroimidazo[1,2-a]pyridin-3-yl)pyrimidin-4-yl)piperazine-1-carboxylate (339 mg, 0.54 mmol, Preparation 76a) and trifluoroacetic acid (414 μL, 5.37 mmol) following the procedure described in Preparation 74b. The title compound was obtained as a white solid (238 mg, 84%) which was used in the next step without further purification.

LRMS (m/z): 531 (M+1)⁺

¹H NMR [symbol] (CDCl₃): 1.7-2.0 (m, 4H), 2.3-2.9 (m, 5H), 3.2-3.8 (m, 9H), 5.1 (d, 1H), 5.2 (s, 2H), 5.4 (s, 1H), 7.2 (m, 1H), 7.3 (m, 5H), 7.6 (dd, 1H), 8.4 (s, 1H), 9.9 (dd, 1H).

### c) (R)-Benzyl 4-(6-(1-(2-cyanoacetyl)piperidin-3-ylamino)-2-(6-fluoroimidazo[1,2-a]pyridin-3-yl)pyrimidin-4-yl)piperazine-1-carboxylate

3-[(2,5-dioxopyrrolidin-1-yl)oxy]-3-oxopropanenitrile (123 mg, 0.68 mmol, prepared as described in BE875054(A1)) and triethylamine (94 μL, 0.67 mmol) were added to a solution of (R)-benzyl 4-(2-(6-fluoroimidazo[1,2-a]pyridin-3-yl)-6-(piperidin-3-ylamino) pyrimidin-4-yl)piperazine-1-carboxylate (238 mg, 0.45 mmol, Preparation 76b) in methylene chloride (10 mL) and the resulting mixture was stirred at room temperature for 2 hours. The solvent was then evaporated and the residue partitioned between water and chloroform. The organic layer was separated, washed with water and brine, dried over magnesium sulphate and the solvent evaporated. The crude was purified by flash chromatography (dichloromethane to 93:7 dichloromethane/methanol) to yield 204 mg (76%) of the title product as a white solid.

LRMS (m/z): 598 (M+1)⁺

¹H NMR [symbol] (CDCl₃): 1.7-2.2 (m, 4H), 3.3-3.8 (m, 14H), 4.1-4.8 (m, 2H), 5.2 (s, 2H), 5.5 (s, 1H), 7.2 (m, 1H), 7.4 (m, 5H), 7.7 (m, 1H), 8.5 (d, 1H), 9.8 (m, 1H).

### Preparation 77

### (R)-2-(6-Fluoroimidazo[1,2-a]pyridin-3-yl)-6-morpholino-N-(piperidin-3-yl)pyrimidin-4-amine

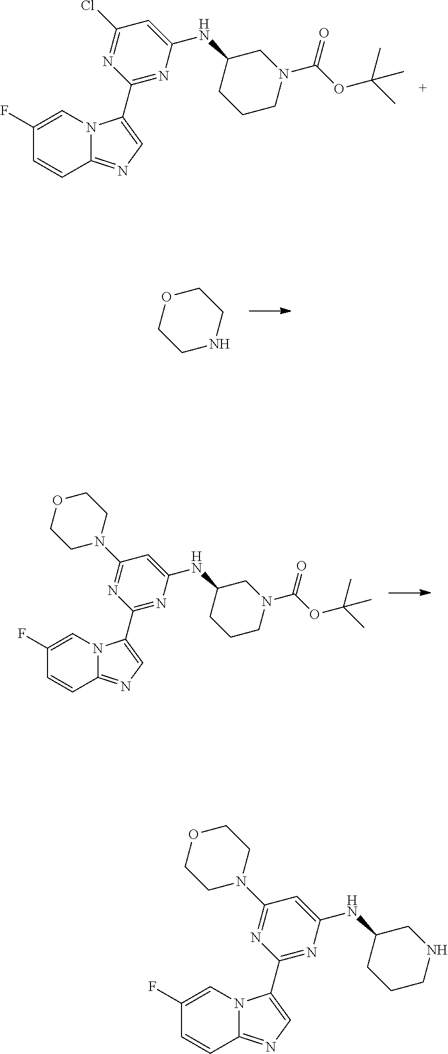 [SMILES: CC(C)(C)OC(=O)N1CCC[C@@H](Nc2cc(Cl)nc(-c3cnc4ccc(F)cn34)n2)C1 ; C1COCCN1 ; CC(C)(C)OC(=O)N1CCC[C@@H](Nc2cc(N3CCOCC3)nc(-c3cnc4ccc(F)cn34)n2)C1 ; Fc1ccc2ncc(-c3nc(N[C@@H]4CCCNC4)cc(N4CCOCC4)n3)n2c1]

### a) (R)-tert-Butyl 3-(2-(6-fluoroimidazo[1,2-a]pyridin-3-yl)-6-morpholinopyrimidin-4-ylamino)piperidine-1-carboxylate

Prepared from (R)-tert-butyl 3-(6-chloro-2-(6-fluoroimidazo[1,2-a]pyridin-3-yl)pyrimidin-4-ylamino)piperidine-1-carboxylate (100 mg, 0.22 mmol, Preparation 73) and morpholine (100 μL, 1.12 mmol) following the procedure described in Preparation 76a. The crude product was purified by flash chromatography (dichloromethane to 92:8 dichloromethane/methanol) to yield 85 mg (76%) of the title compound as a yellowish solid.

LRMS (m/z): 498 (M+1)⁺

¹H NMR [symbol] (CDCl₃): 1.4 (s, 9H), 1.6-2.1 (m, 5H), 2.9-3.3 (m, 4H), 3.6 (m, 4H), 3.8 (m, 4H), 4.8 (bs, 1H), 5.4 (bs, 1H), 7.2 (m, 1H), 7.7 (dd, 1H), 8.5 (s, 1H), 9.8 (dd, 1H).

### b) (R)-2-(6-Fluoroimidazo[1,2-a]pyridin-3-yl)-6-morpholino-N-(piperidin-3-yl)pyrimidin-4-amine

Prepared from (R)-tert-butyl 3-(2-(6-fluoroimidazo[1,2-a]pyridin-3-yl)-6-morpholino pyrimidin-4-ylamino)piperidine-1-carboxylate (85 mg, 0.17 mmol, Preparation 77a) and trifluoroacetic acid (66 μL, 0.86 mmol) following the procedure described in Preparation 74b. The title compound was obtained as a white solid (68 mg, 100%) which was used in the next step without further purification.

LRMS (m/z): 398 (M+1)⁺

¹H NMR [symbol] (CDCl₃): 1.6-2.0 (m, 4H), 2.7-3.0 (m, 4H), 3.2-3.4 (m, 2H), 3.6 (m, 4H), 3.8 (m, 4H), 5.1 (bs, 1H), 5.4 (s, 1H), 7.2 (m, 1H), 7.6 (dd, 1H), 8.4 (s, 1H), 9.8 (dd, 1H).

### Preparation 78

### (R)-2-(6-Fluoroimidazo[1,2-a]pyridin-3-yl)-N⁴,N⁴-dimethyl-N⁶-(piperidin-3-yl)pyrimidine-4,6-diamine

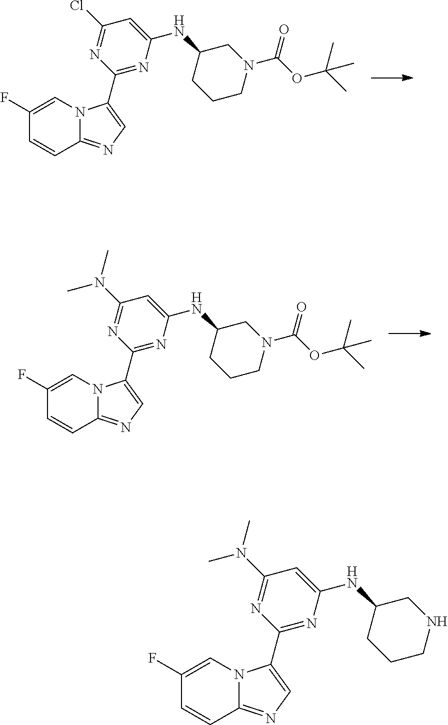 [SMILES: CC(C)(C)OC(=O)N1CCC[C@@H](Nc2cc(Cl)nc(-c3cnc4ccc(F)cn34)n2)C1 ; CN(C)c1cc(N[C@@H]2CCCN(C(=O)OC(C)(C)C)C2)nc(-c2cnc3ccc(F)cn23)n1 ; CN(C)c1cc(N[C@@H]2CCCNC2)nc(-c2cnc3ccc(F)cn23)n1]

### a) (R)-tert-Butyl 3-(6-(dimethylamino)-2-(6-fluoroimidazo[1,2-a]pyridin-3-yl)pyrimidin-4-ylamino)piperidine-1-carboxylate

Prepared from (R)-tert-butyl 3-(6-chloro-2-(6-fluoroimidazo[1,2-a]pyridin-3-yl)pyrimidin-4-ylamino)piperidine-1-carboxylate (100 mg, 0.22 mmol, Preparation 73) and dimethylamine (0.56 mL of a 2M solution in methanol, 1.12 mmol) in N,N′-dimethylformamide (2 mL) following the procedure described in Preparation 76a. The crude product was purified by flash chromatography (dichloromethane to 92:8 dichloromethane/methanol) to yield 59 mg (49%) of the title product as a solid.

LRMS (m/z): 456 (M+1)⁺

¹H NMR [symbol] (CDCl₃): 1.4 (s, 9H), 1.6-2.1 (m, 4H), 3.2 (s, 6H), 3.1 (m, 1H), 3.6-3.9 (m, 4H), 4.7 (bs, 1H), 5.3 (m, 1H), 7.2 (m, 1H), 7.6 (dd, 1H), 8.5 (dd, 1H), 9.9 (dd, 1H).

### b) (R)-2-(6-Fluoroimidazo[1,2-a]pyridin-3-yl)-N⁴,N⁴-dimethyl-N⁶-(piperidin-3-yl)pyrimidine-4,6-diamine

Prepared from (R)-tert-butyl 3-(6-(dimethylamino)-2-(6-fluoroimidazo[1,2-a]pyridin-3-yl)pyrimidin-4-ylamino)piperidine-1-carboxylate (59 mg, 0.13 mmol, Preparation 78a) and trifluoroacetic acid (50 μL, 0.65 mmol) following the procedure described in Preparation 74b. The title compound was obtained as a white solid (46 mg, 100%) which was used in the next step without further purification.

LRMS (m/z): 356 (M+1)⁺

### Preparation 79

### (R)-2-(6-Fluoroimidazo[1,2-a]pyridin-3-yl)-N⁴-(2-morpholinoethyl)-N⁶-(piperidin-3-yl)pyrimidine-4,6-diamine

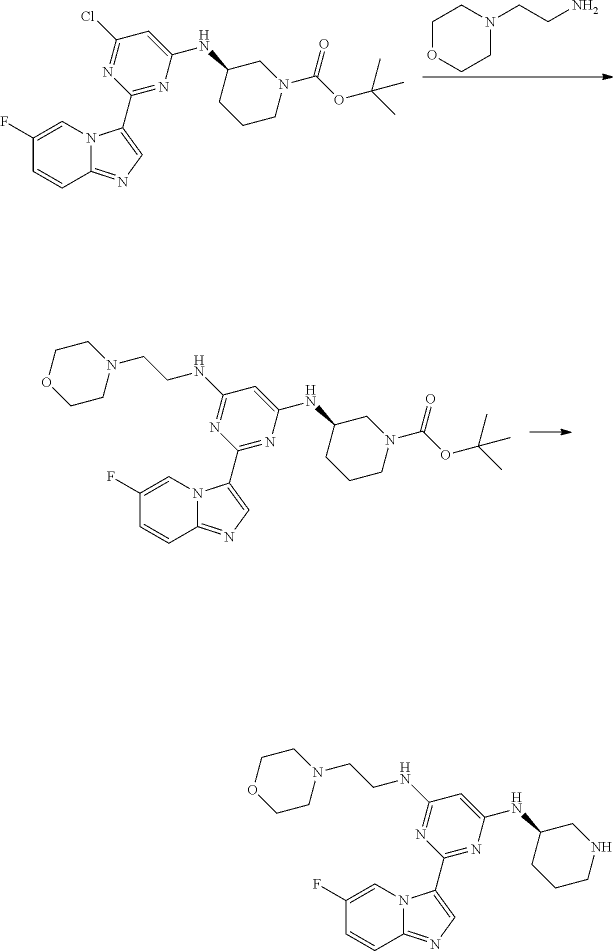

### a) (R)-tert-Butyl 3-(2-(6-fluoroimidazo[1,2-a]pyridin-3-yl)-6-(2-morpholino ethylamino)pyrimidin-4-ylamino)piperidine-1-carboxylate

Prepared from (R)-tert-butyl 3-(6-chloro-2-(6-fluoroimidazo[1,2-a]pyridin-3-yl)pyrimidin-4-ylamino)piperidine-1-carboxylate (100 mg, 0.22 mmol, Preparation 73) and 2-morpholinoethanamine (147 μL, 1.12 mmol) in N-methylpirrolidone (2 mL) following the procedure described in Preparation 76a. The reaction mixture was heated under microwave irradiation at 130° C. for 6 hours and then at 140° C. for additional 4 hours. The crude product was purified by flash chromatography (dichloromethane to 88:12 dichloromethane/methanol) to yield 56 mg (46%) of the title product as a white solid.

LRMS (m/z): 541 (M+1)⁺

¹H NMR [symbol] (CDCl₃): 1.4 (s, 4H), 1.5 (s, 9H), 1.6 (m, 2H), 1.6-2.1 (m, 2H), 2.5 (m, 2H), 2.6 (m, 1H), 3.4-3.8 (m, 5H), 4.7 (d, 1H), 5.3 (bs, 1H), 7.2 (dd, 1H), 7.6 (dd, 1H), 8.5 (s, 1H), 9.9 (dd, 1H).

### b) (R)-2-(6-Fluoroimidazo[1,2-a]pyridin-3-yl)-N⁴-(2-morpholinoethyl)-N⁶-(piperidin-3-yl)pyrimidine-4,6-diamine

Prepared from (R)-tert-butyl 3-(2-(6-fluoroimidazo[1,2-a]pyridin-3-yl)-6-(2-morpholino ethylamino)pyrimidin-4-ylamino)piperidine-1-carboxylate (56 mg, 0.10 mmol, Preparation 79a) and trifluoroacetic acid (80 μL, 1.04 mmol) following the procedure described in Preparation 74b. The title compound was obtained as a yellowish solid (45 mg, 100%) which was used in the next step without further purification.

LRMS (m/z): 441 (M+1)⁺

### Preparation 80

### (R)-2-(6-Fluoroimidazo[1,2-a]pyridin-3-yl)-N⁴-(2-methoxyethyl)-N⁶-(piperidin-3-yl)pyrimidine-4,6-diamine

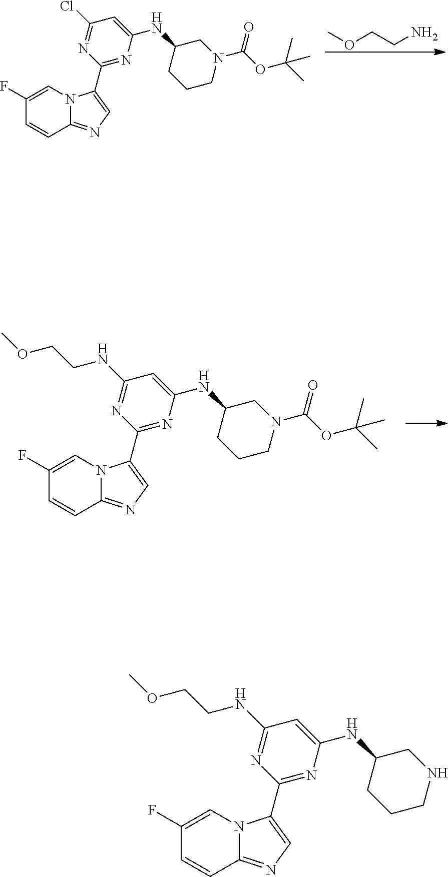

### a) (R)-tert-Butyl 3-(2-(6-fluoroimidazo[1,2-a]pyridin-3-yl)-6-(2-methoxyethylamino) pyrimidin-4-ylamino)piperidine-1-carboxylate

Prepared from (R)-tert-butyl 3-(6-chloro-2-(6-fluoroimidazo[1,2-a]pyridin-3-yl)pyrimidin-4-ylamino)piperidine-1-carboxylate (250 mg, 0.56 mmol, Preparation 73) and 2-methoxyethanamine (210 mg, 2.80 mmol) in N-methylpirrolidone (2 mL) following the procedure described in Preparation 76a. The reaction mixture was heated under microwave irradiation at 140° C. for 2 hours. The crude product was purified by flash chromatography (dichloromethane to 92:8 dichloromethane/methanol) to yield 156 mg (57%) of the title product as a white solid.

LRMS (m/z): 486 (M+1)⁺

¹H NMR [symbol] (CDCl₃): 1.4 (s, 9H), 1.6-2.0 (m, 4H), 3.2 (bs, 2H), 3.4 (s, 3H), 3.5-4.1 (m, 6H), 4.7 (bs, 1H), 5.0 (bs, 1H), 5.3 (bs, 1H), 7.2 (m, 1H), 7.6 (dd, 1H), 8.4 (s, 1H), 9.9 (dd, 1H).

### b) (R)-2-(6-Fluoroimidazo[1,2-a]pyridin-3-yl)-N⁴-(2-methoxyethyl)-N⁶-(piperidin-3-yl)pyrimidine-4,6-diamine

Prepared from (R)-tert-butyl 3-(2-(6-fluoroimidazo[1,2-a]pyridin-3-yl)-6-(2-methoxyethyl amino)pyrimidin-4-ylamino)piperidine-1-carboxylate (156 mg, 0.32 mmol, Preparation 80a) and trifluoroacetic acid (124 μL, 1.61 mmol) following the procedure described in Preparation 74b. The title compound was obtained as a yellowish solid (123 mg, 100%) which was used in the next step without further purification.

LRMS (m/z): 386 (M+1)⁺

¹H NMR [symbol] (CDCl₃): 1.6-2.0 (m, 5H), 2.6-2.8 (m, 2H), 2.9 (m, 1H), 3.2 (dd, 1H), 3.4 (s, 3H), 3.5-3.7 (m, 4H), 3.7 (bs, 1H), 5.0 (bs, 2H), 5.2 (s, 1H), 7.2 (m, 1H), 7.6 (dd, 1H), 8.4 (dd, 1H), 10.0 (dd, 1H).

### Preparation 81

### (S)-Methyl 1-(6-chloro-2-(6-fluoroimidazo[1,2-a]pyridin-3-yl)pyrimidin-4-yl)pyrrolidine-2-carboxylate

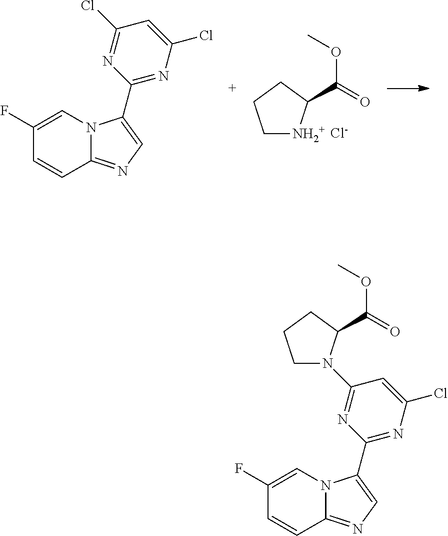

(S)-2-(Methoxycarbonyl)pyrrolidinium chloride (393 mg, 2.37 mmol) and triethylamine (607 μL, 4.35 mmol) were added to a suspension of 3-(4,6-dichloropyrimidin-2-yl)-6-fluoroimidazo[1,2-a]pyridine (560 mg, 1.98 mmol, Preparation 72b) in ethanol (20 mL) and the resulting mixture was heated to reflux for 3 hours. The solvent was evaporated and the residue was partitioned between water and chloroform. The organic layer was separated, washed with water and brine, dried over magnesium sulphate and the solvent evaporated to give 600 mg (81%) of the title product, which was used in the next step without further purification.

LRMS (m/z): 376 (M+1)⁺

¹H NMR [symbol] (CDCl₃): 2.1-2.4 (m, 4H), 3.4-3.7 (m, 2H), 3.8 (s, 3H), 4.8 (dd, 1H), 6.3 (s, 1H), 7.3 (m, 1H), 7.7 (dd, 1H), 8.5 (s, 1H), 9.7 (s, 1H).

### Preparation 82

### (S)-1-(2-(6-Fluoroimidazo[1,2-a]pyridin-3-yl)-6-((R)-piperidin-3-ylamino)pyrimidin-4-yl)pyrrolidine-2-carboxylic acid

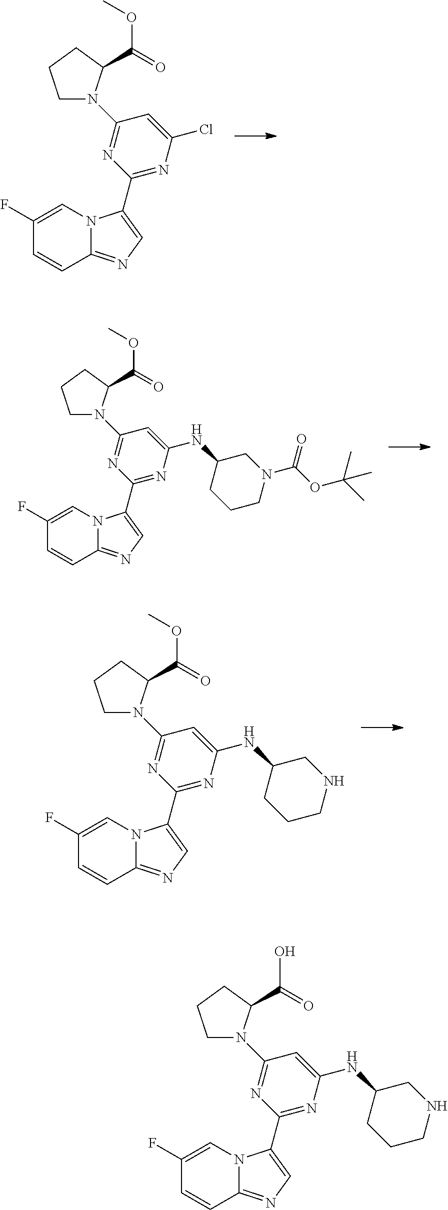 [SMILES: COC(=O)[C@@H]1CCCN1c1cc(Cl)nc(-c2cnc3ccc(F)cn23)n1 ; COC(=O)[C@@H]1CCCN1c1cc(N[C@@H]2CCCN(C(=O)OC(C)(C)C)C2)nc(-c2cnc3ccc(F)cn23)n1 ; COC(=O)[C@@H]1CCCN1c1cc(N[C@@H]2CCCNC2)nc(-c2cnc3ccc(F)cn23)n1 ; O=C(O)[C@@H]1CCCN1c1cc(N[C@@H]2CCCNC2)nc(-c2cnc3ccc(F)cn23)n1]

### a) (R)-tert-Butyl 3-(2-(6-fluoroimidazo[1,2-a]pyridin-3-yl)-6-((S)-2-(methoxy carbonyl)pyrrolidin-1-yl)pyrimidin-4-ylamino)piperidine-1-carboxylate

(R)-tert-Butyl 3-aminopiperidine-1-carboxylate (800 mg, 4.0 mmol) was added to a suspension of (S)-methyl 1-(6-chloro-2-(6-fluoroimidazo[1,2-a]pyridin-3-yl)pyrimidin-4-yl)pyrrolidine-2-carboxylate (600 mg, 1.60 mmol, Preparation 81) in ethanol (5 mL) and the resulting mixture was heated under microwave irradiation at 100° C. for 16 hours. The solvent was then evaporated and the residue was partitioned between water and chloroform. The organic layer was separated, washed with water and brine, dried over magnesium sulphate and the solvent evaporated. The crude product was purified by flash chromatography (dichloromethane to 8:2 dichloromethane/methanol) to yield 140 mg (16%) of the title compound.

LRMS (m/z): 540 (M+1)⁺

### b) (S)-Methyl 1-(2-(6-fluoroimidazo[1,2-a]pyridin-3-yl)-6-((R)-piperidin-3-ylamino) pyrimidin-4-yl)pyrrolidine-2-carboxylate

Prepared from (R)-tert-butyl 3-(2-(6-fluoroimidazo[1,2-a]pyridin-3-yl)-64(S)-2-(methoxy carbonyl)pyrrolidin-1-yl)pyrimidin-4-ylamino)piperidine-1-carboxylate (140 mg, 0.26 mmol, Preparation 82a) and trifluoroacetic acid (100 μL, 1.30 mmol) following the procedure described in Preparation 74b. The title compound was obtained as a yellowish solid (103 mg, 90%) which was used in the next step without further purification.

LRMS (m/z): 440 (M+1)⁺

### c) (S)-1-(2-(6-Fluoroimidazo[1,2-a]pyridin-3-yl)-6-((R)-piperidin-3-ylamino) pyrimidin-4-yl)pyrrolidine-2-carboxylic acid

A 2M aqueous solution of sodium hydroxide (365 μL, 0.73 mmol) was added to a solution of (S)-methyl 1-(2-(6-fluoroimidazo[1,2-a]pyridin-3-yl)-6-((R)-piperidin-3-yl amino)pyrimidin-4-yl)pyrrolidine-2-carboxylate (123 mg, 0.24 mmol, Preparation 82b) in methanol (5 mL) and the resulting mixture was stirred at room temperature overnight. The solvent was then evaporated and the residue was redissolved with water. A 2M aqueous solution of hydrochloric acid was added until neutral pH was reached and a white solid precipitated, which was filtered and dried to yield 30 mg (25%) of the title compound.

LRMS (m/z): 426 (M+1)⁺

### Preparation 83

### (R)-2-(6-Fluoroimidazo[1,2-a]pyridin-3-yl)-6-methoxy-N-(piperidin-3-yl)pyrimidin-4-amine

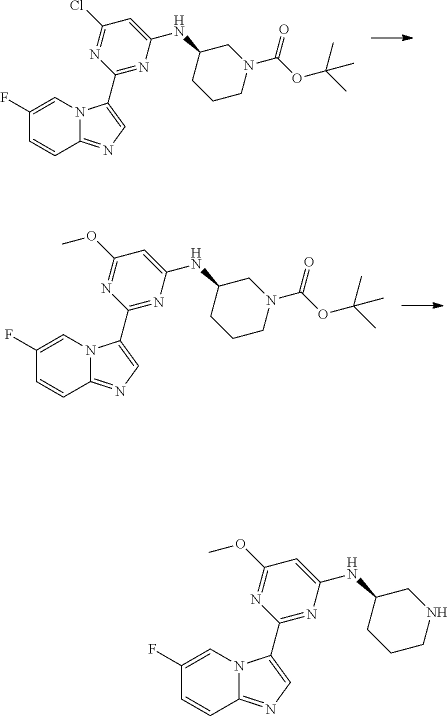 [SMILES: CC(C)(C)OC(=O)N1CCC[C@@H](Nc2cc(Cl)nc(-c3cnc4ccc(F)cn34)n2)C1 ; COc1cc(N[C@@H]2CCCN(C(=O)OC(C)(C)C)C2)nc(-c2cnc3ccc(F)cn23)n1 ; COc1cc(N[C@@H]2CCCNC2)nc(-c2cnc3ccc(F)cn23)n1]

### a) (R)-tert-Butyl 3-(2-(6-fluoroimidazo[1,2-a]pyridin-3-yl)-6-methoxypyrimidin-4-yl amino)piperidine-1-carboxylate

(R)-tert-Butyl 3-(6-chloro-2-(6-fluoroimidazo[1,2-a]pyridin-3-yl)pyrimidin-4-ylamino) piperidine-1-carboxylate (132 mg, 0.30 mmol, Preparation 73) was added to a solution of sodium methoxide (prepared from 15 mg (0.65 mmol) of sodium in 5 mL of methanol) and the resulting mixture was heated to reflux overnight. The solvent was then evaporated and the residue was partitioned between water and chloroform. The organic layer was separated, washed with water and brine, dried over magnesium sulphate and the solvent evaporated. The crude product was purified by flash chromatography (dichloromethane to 95:5 dichloromethane/methanol) to yield 117 mg (90%) of the title compound as a white solid.

LRMS (m/z): 443 (M+1)⁺

### b) (R)-2-(6-Fluoroimidazo[1,2-a]pyridin-3-yl)-6-methoxy-N-(piperidin-3-yl)pyrimidin-4-amine

Prepared from (R)-tert-butyl 3-(2-(6-fluoroimidazo[1,2-a]pyridin-3-yl)-6-methoxy pyrimidin-4-ylamino)piperidine-1-carboxylate (117 mg, 0.26 mmol, Preparation 83a) and trifluoroacetic acid (102 μL, 1.32 mmol) following the procedure described in Preparation 74b. The title compound was obtained as a white solid (90 mg, 100%) which was used in the next step without further purification.

LRMS (m/z): 343 (M+1)⁺

¹H NMR [symbol] (CDCl₃): 1.5-2.0 (m, 5H), 2.7-3.2 (m, 4H), 3.7 (bs, 1H), 4.0 (d, 3H), 5.2 (d, 1H), 5.6 (s, 1H), 7.2 (m, 1H), 7.7 (dd, 1H), 8.5 (s, 1H), 9.9 (dd, 1H).

### Preparation 84

### (R)-2-(6-Fluoroimidazo[1,2-a]pyridin-3-yl)-N-(piperidin-3-yl)-6-(2-(pyrrolidin-1-yl)ethoxy)pyrimidin-4-amine

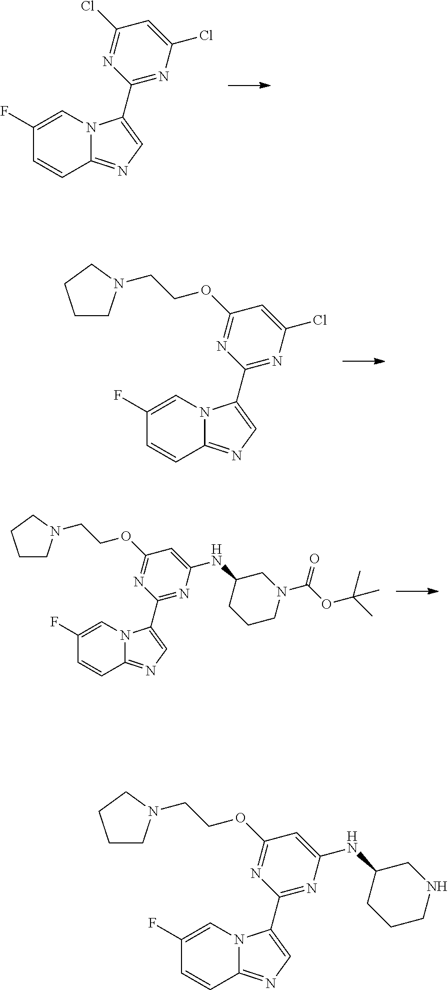 [SMILES: Fc1ccc2ncc(-c3nc(Cl)cc(Cl)n3)n2c1 ; Fc1ccc2ncc(-c3nc(Cl)cc(OCCN4CCCC4)n3)n2c1 ; CC(C)(C)OC(=O)N1CCC[C@@H](Nc2cc(OCCN3CCCC3)nc(-c3cnc4ccc(F)cn34)n2)C1 ; Fc1ccc2ncc(-c3nc(N[C@@H]4CCCNC4)cc(OCCN4CCCC4)n3)n2c1]

### a) 3-(4-Chloro-6-(2-(pyrrolidin-1-yl)ethoxy)pyrimidin-2-yl)-6-fluoroimidazo[1,2-a]pyridine

2-(Pyrrolidin-1-yl)ethanol (186 μL, 1.59 mmol) was added to a suspension of sodium hydride (64 mg of a 60% suspension in mineral oil, 1.60 mmol) in anhydrous tetrahydrofuran (5 mL) and the resulting mixture was stirred at room temperature for 30 minutes. A solution of 3-(4,6-dichloropyrimidin-2-yl)-6-fluoroimidazo[1,2-a]pyridine (300 mg, 1.06 mmol, Preparation 72b) in tetrahydrofuran (10 mL) was then added and the resulting reaction mixture was stirred overnight at room temperature. The solvent was evaporated and the residue was partitioned between water and methylene chloride. The organic phase was separated, washed with water and brine, dried over magnesium sulphate and the solvent evaporated. The crude product was purified by flash chromatography (dichloromethane to 9:1 dichloromethane/methanol) to yield 300 mg (78%) of the title product as a yellowish solid.

LRMS (m/z): 362 (M+1)⁺

¹H NMR [symbol] (CDCl₃): 1.8 (m, 4H), 2.6 (m, 4H), 2.9 (t, 2H), 4.6 (t, 2H), 6.6 (s, 1H), 7.3 (m, 1H), 7.7 (dd, 1H), 8.6 (s, 1H), 9.8 (dd, 1H).

### b) (R)-tert-Butyl 3-(2-(6-fluoroimidazo[1,2-a]pyridin-3-yl)-6-(2-(pyrrolidin-1-yl)ethoxy)pyrimidin-4-ylamino)piperidine-1-carboxylate

Prepared from 3-(4-chloro-6-(2-(pyrrolidin-1-yl)ethoxy)pyrimidin-2-yl)-6-fluoroimidazo[1,2-a]pyridine (300 mg, 0.83 mmol, Preparation 84a) and (R)-tert-butyl 3-aminopiperidine-1-carboxylate (415 mg, 2.07 mmol) in N-methylpirrolidone (4 mL) following the procedure described in Preparation 76a. The mixture was heated under microwave irradiation at 120° C. for 1 hour. The crude product was purified by flash chromatography (dichloromethane to 9:1 dichloromethane/methanol) followed by a second purification by reverse phase chromatography (C-18 silica from Waters, water/1:1 acetonitrile-methanol as eluents [0.1% v/v formic acid buffered] 0% to 100%). 160 mg (37%) of the title compound were obtained as a white solid.

LRMS (m/z): 526 (M+1)⁺

### c) (R)-2-(6-Fluoroimidazo[1,2-a]pyridin-3-yl)-N-(piperidin-3-yl)-6-(2-(pyrrolidin-1-yl)ethoxy)pyrimidin-4-amine

Prepared from (R)-tert-butyl 3-(2-(6-fluoroimidazo[1,2-a]pyridin-3-yl)-6-(2-(pyrrolidin-1-yl)ethoxy)pyrimidin-4-ylamino)piperidine-1-carboxylate (160 mg, 0.30 mmol, Preparation 84b) and trifluoroacetic acid (235 μL, 3.05 mmol) following the procedure described in Preparation 74b. The title compound was obtained as a white semisolid (111 mg, 86%) which was used in the next step without further purification.

LRMS (m/z): 426 (M+1)⁺

### Preparation 85

### (R)-2-(6-Fluoroimidazo[1,2-a]pyridin-3-yl)-6-(2-morpholinoethoxy)-N-(piperidin-3-yl)pyrimidin-4-amine

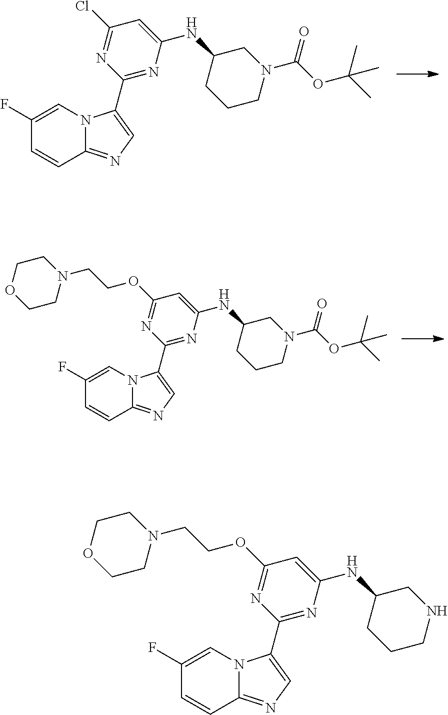 [SMILES: CC(C)(C)OC(=O)N1CCC[C@@H](Nc2cc(Cl)nc(-c3cnc4ccc(F)cn34)n2)C1 ; CC(C)(C)OC(=O)N1CCC[C@@H](Nc2cc(OCCN3CCOCC3)nc(-c3cnc4ccc(F)cn34)n2)C1 ; Fc1ccc2ncc(-c3nc(N[C@@H]4CCCNC4)cc(OCCN4CCOCC4)n3)n2c1]

### a) (R)-tert-Butyl 3-(2-(6-fluoroimidazo[1,2-a]pyridin-3-yl)-6-(2-morpholinoethoxy)pyrimidin-4-ylamino)piperidine-1-carboxylate

2-Morpholinoethanol (73 μL, 0.65 mmol) was added to a suspension of sodium hydride (24 mg of a 60% suspension in mineral oil, 0.60 mmol) in tetrahydrofuran (5 mL) and the resulting mixture was stirred at room temperature for 30 minutes. (R)-Tert-butyl 3-(6-chloro-2-(6-fluoroimidazo[1,2-a]pyridin-3-yl)pyrimidin-4-ylamino)piperidine-1-carboxylate (180 mg, 0.40 mmol, Preparation 73) was then added and the resulting reaction mixture was stirred at reflux for 40 hours. The solvent was evaporated and the residue was partitioned between water and chloroform. The organic phase was separated, washed with water and brine, dried over magnesium sulphate and the solvent evaporated. The crude product was purified by flash chromatography (dichloromethane to 94:6 dichloromethane/methanol) to yield 147 mg (67%) of the title compound as a solid.

LRMS (m/z): 542 (M+1)⁺

¹H NMR [symbol] (CDCl₃): 1.4 (s, 9H), 1.6-2.1 (m, 4H), 2.6 (m, 4H), 2.8 (t, 2H), 3.1-3.3 (m, 2H), 3.6 (m, 2H), 3.7 (m, 4H), 3.9 (m, 1H), 4.6 (t, 2H), 4.9 (s, 1H), 5.6 (s, 1H), 7.2 (m, 1H), 7.7 (dd, 1H), 8.5 (s, 1H), 9.8 (dd, 1H).

### b) (R)-2-(6-Fluoroimidazo[1,2-a]pyridin-3-yl)-6-(2-morpholinoethoxy)-N-(piperidin-3-yl)pyrimidin-4-amine

Prepared from (R)-tert-butyl 3-(2-(6-fluoroimidazo[1,2-a]pyridin-3-yl)-6-(2-morpholino ethoxy)pyrimidin-4-ylamino)piperidine-1-carboxylate (147 mg, 0.27 mmol, Preparation 85a) and trifluoroacetic acid (210 μL, 2.73 mmol) following the procedure described in Preparation 74b. The crude product was purified by flash chromatography (dichloromethane to 85:15 dichloromethane/methanol) to give 107 mg (89%) of the title compound as a white solid.

LRMS (m/z): 442 (M+1)⁺

¹H NMR [symbol] (CDCl₃): 1.6-2.0 (m, 4H), 2.6 (m, 4H), 2.8-2.9 (m, 5H), 3.2 (dd, 1H), 3.6 (bs, 2H), 3.7 (m, 4H), 4.6 (t, 2H), 5.6 (s, 1H), 5.7 (bs, 1H), 7.2 (m, 1H), 7.7 (dd, 1H), 8.5 (s, 1H), 9.9 (dd, 1H).

### Preparation 86

### (R)-2-(6-Fluoroimidazo[1,2-a]pyridin-3-yl)-6-(2-methoxyethoxy)-N-(piperidin-3-yl)pyrimidin-4-amine

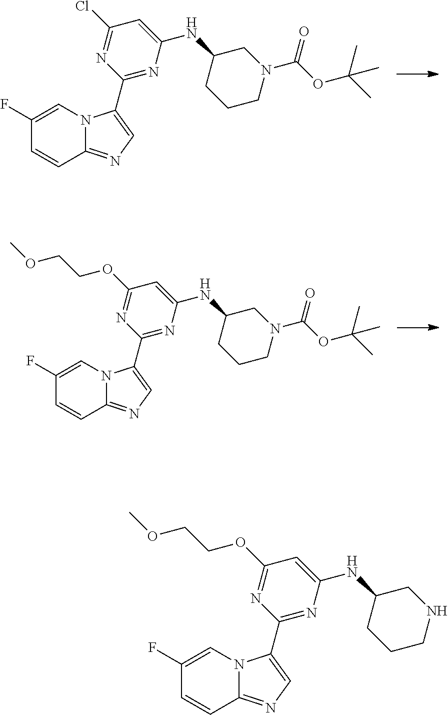

### a) (R)-tert-Butyl 3-(2-(6-fluoroimidazo[1,2-a]pyridin-3-yl)-6-(2-methoxyethoxy)pyrimidin-4-ylamino)piperidine-1-carboxylate

Prepared from (R)-tert-butyl 3-(6-chloro-2-(6-fluoroimidazo[1,2-a]pyridin-3-yl)pyrimidin-4-ylamino)piperidine-1-carboxylate (200 mg, 0.45 mmol, Preparation 73), 2-methoxyethanol (170 μL, 2.23 mmol) and sodium hydride (90 mg of a 60% suspension in mineral oil, 2.23 mmol) following the procedure described in Preparation 85a. The crude product was purified by reverse phase chromatography (C-18 silica from Waters, water/1:1 acetonitrile-methanol as eluents [0.1% v/v formic acid buffered] 0% to 100%) to yield 63 mg (29%) of the title compound as a solid.

LRMS (m/z): 487 (M+1)⁺

¹H NMR [symbol] (CDCl₃): 1.4 (s, 9H), 1.6-2.1 (m, 5H), 3.2 (m, 2H), 3.4 (s, 3H), 3.6 (m, 1H), 3.8 (m, 2H), 3.9 (bs, 1H), 4.6 (m, 2H), 4.9 (bs, 1H), 5.7 (s, 1H), 7.2 (m, 1H), 7.7 (dd, 1H), 8.5 (s, 1H), 9.8 (dd, 1H).

### b) (R)-2-(6-Fluoroimidazo[1,2-a]pyridin-3-yl)-6-(2-methoxyethoxy)-N-(piperidin-3-yl)pyrimidin-4-amine

Prepared from (R)-tert-butyl 3-(2-(6-fluoroimidazo[1,2-a]pyridin-3-yl)-6-(2-methoxy ethoxy)pyrimidin-4-ylamino)piperidine-1-carboxylate (63 mg, 0.13 mmol, Preparation 86a) and trifluoroacetic acid (50 μL, 0.65 mmol) following the procedure described in Preparation 74b. The title compound was obtained as a brownish solid (50 mg, 100%) which was used in the next step without further purification.

LRMS (m/z): 387 (M+1)⁺

### Preparation 87

### (R)-2-(6-Fluoroimidazo[1,2-a]pyridin-3-yl)-6-(piperidin-3-ylamino)pyrimidine-4-carbonitrile

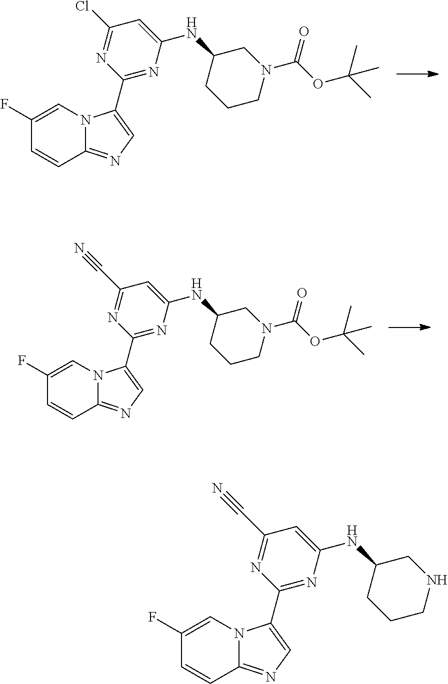 [SMILES: CC(C)(C)OC(=O)N1CCC[C@@H](Nc2cc(Cl)nc(-c3cnc4ccc(F)cn34)n2)C1 ; CC(C)(C)OC(=O)N1CCC[C@@H](Nc2cc(C#N)nc(-c3cnc4ccc(F)cn34)n2)C1 ; N#Cc1cc(N[C@@H]2CCCNC2)nc(-c2cnc3ccc(F)cn23)n1]

### a) (R)-tert-Butyl 3-(6-cyano-2-(6-fluoroimidazo[1,2-a]pyridin-3-yl)pyrimidin-4-ylamino)piperidine-1-carboxylate

Dicyanozinc (220 mg, 1.87 mmol) and tetrakis(triphenylphosphine)palladium (0) (286 mg, 0.25 mmol) were added to a solution of (R)-tert-butyl 3-(6-chloro-2-(6-fluoroimidazo[1,2-a]pyridin-3-yl)pyrimidin-4-ylamino)piperidine-1-carboxylate (1.1 g, 2.46 mmol, Preparation 73) in N,N′-dimethylformamide (30 mL) and the resulting mixture was stirred at 130° C. for 1 hour under argon atmosphere. The solvent was then evaporated and the residue was partitioned between water and chloroform. The organic phase was separated, washed with water and brine, dried over magnesium sulphate and the solvent evaporated. The crude product was purified by flash chromatography (dichloromethane to 95:5 dichloromethane/methanol) to yield 1.14 g (100%) of the title product as a yellowish solid.

LRMS (m/z): 438 (M+1)⁺

¹H NMR [symbol] (CDCl₃): 1.5 (s, 9H), 1.6-2.0 (m, 4H), 3.4-4.3 (m, 5H), 5.3 (s, 1H), 5.5 (bs, 1H), 6.5 (s, 1H), 7.3 (m, 1H), 7.7 (dd, 1H), 8.6 (s, 1H).

### b) (R)-2-(6-Fluoroimidazo[1,2-a]pyridin-3-yl)-6-(piperidin-3-ylamino)pyrimidine-4-carbonitrile

Prepared from (R)-tert-butyl 3-(6-cyano-2-(6-fluoroimidazo[1,2-a]pyridin-3-yl)pyrimidin-4-ylamino)piperidine-1-carboxylate (100 mg, 0.23 mmol, Preparation 87a) and trifluoroacetic acid (88 μL, 1.14 mmol) following the procedure described in Preparation 74b. The title compound was obtained as a white solid (77 mg, 100%) which was used in the next step without further purification.

LRMS (m/z): 338 (M+1)⁺

¹H NMR [symbol] (CDCl₃): 1.5-1.9 (m, 5H), 2.9 (m, 4H), 3.1 (bs, 1H), 6.1 (bs, 1H), 6.5 (s, 1H), 7.3 (m, 1H), 7.7 (dd, 1H), 8.5 (s, 1H), 9.8 (dd, 1H).

### Preparation 88

### (R)-2-(6-Fluoroimidazo[1,2-a]pyridin-3-yl)-N-(piperidin-3-yl)-6-(2H-tetrazol-5-yl)pyrimidin-4-amine

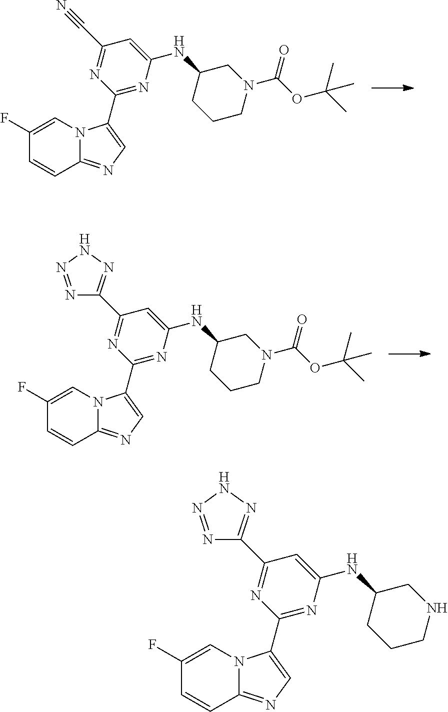 [SMILES: CC(C)(C)OC(=O)N1CCC[C@@H](Nc2cc(C#N)nc(-c3cnc4ccc(F)cn34)n2)C1 ; CC(C)(C)OC(=O)N1CCC[C@@H](Nc2cc(-c3nn[nH]n3)nc(-c3cnc4ccc(F)cn34)n2)C1 ; Fc1ccc2ncc(-c3nc(N[C@@H]4CCCNC4)cc(-c4nn[nH]n4)n3)n2c1]

### a) (R)-tert-Butyl 3-(2-(6-fluoroimidazo[1,2-a]pyridin-3-yl)-6-(2H-tetrazol-5-yl)pyrimidin-4-ylamino)piperidine-1-carboxylate

Azidotrimethylstannane (38 mg, 0.18 mmol) was added to a solution of (R)-tert-butyl 3-(6-cyano-2-(6-fluoroimidazo[1,2-a]pyridin-3-yl)pyrimidin-4-ylamino)piperidine-1-carboxylate (50 mg, 0.11 mmol, Preparation 87a) in toluene (2 mL) and the resulting mixture was heated to reflux for 4 hours. The solvent was then evaporated and the residue was suspended in diethyl ether and stirred overnight. The yellowish solid formed was finally filtered and dried to furnish 50 mg (94%) of the title product.

LRMS (m/z): 481 (M+1)⁺

¹H NMR [symbol] (CDCl₃): 1.4 (s, 9H), 1.6-1.7 (m, 3H), 1.8-2.1 (m, 3H), 3.4-3.6 (m, 3H), 3.9 (bs, 1H), 5.5 (bs, 1H), 7.3 (m, 2H), 7.7 (dd, 1H), 8.6 (s, 1H), 10.1 (d, 1H).

### b) (R)-2-(6-Fluoroimidazo[1,2-a]pyridin-3-yl)-N-(piperidin-3-yl)-6-(2H-tetrazol-5-yl)pyrimidin-4-amine

Prepared from (R)-tert-butyl 3-(2-(6-fluoroimidazo[1,2-a]pyridin-3-yl)-6-(2H-tetrazol-5-yl)pyrimidin-4-ylamino)piperidine-1-carboxylate (200 mg, 0.42 mmol, Preparation 88a) and trifluoroacetic acid (160 μL, 2.08 mmol) following the procedure described in Preparation 74b. 54 mg (54%) of the title compound were obtained and used in the next step without further purification.

LRMS (m/z): 381 (M+1)⁺

### Preparation 89

### (R)-tert-Butyl 3-(2-(6-fluoroimidazo[1,2-a]pyridin-3-yl)-6-(1-methyl-1H-tetrazol-5-yl)pyrimidin-4-ylamino)piperidine-1-carboxylate and (R)-tert-butyl 3-(2-(6-fluoroimidazo[1,2-a]pyridin-3-yl)-6-(2-methyl-2H-tetrazol-5-yl)pyrimidin-4-ylamino) piperidine-1-carboxylate

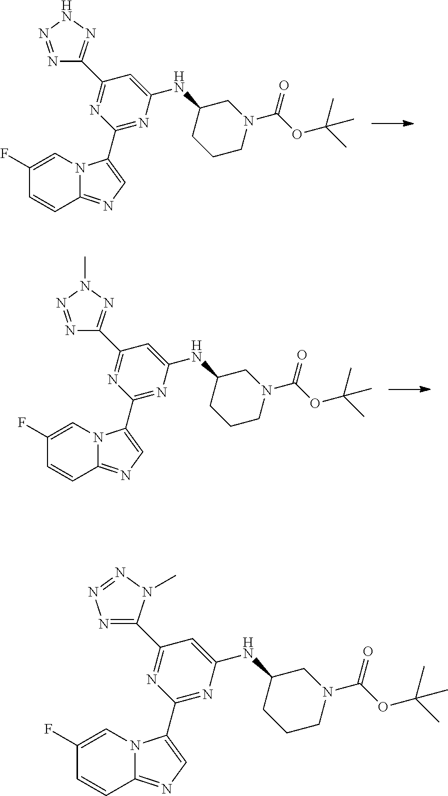 [SMILES: CC(C)(C)OC(=O)N1CCC[C@@H](Nc2cc(-c3nn[nH]n3)nc(-c3cnc4ccc(F)cn34)n2)C1 ; Cn1nnc(-c2cc(N[C@@H]3CCCN(C(=O)OC(C)(C)C)C3)nc(-c3cnc4ccc(F)cn34)n2)n1 ; Cn1nnnc1-c1cc(N[C@@H]2CCCN(C(=O)OC(C)(C)C)C2)nc(-c2cnc3ccc(F)cn23)n1]

Potassium carbonate (190 mg, 1.35 mmol) and methyl iodide (84 μL, 1.35 mmol) were added to a solution of (R)-tert-butyl 3-(2-(6-fluoroimidazo[1,2-a]pyridin-3-yl)-6-(2H-tetrazol-5-yl)pyrimidin-4-ylamino)piperidine-1-carboxylate (590 mg, 1.23 mmol, Preparation 88a) in N,N′-dimethylformamide (5 mL) and the reaction mixture was stirred at room temperature for 2 hours before being poured into 20 mL of water. The yellowish solid that formed was filtered, washed with water and dried. The crude mixture of isomers was purified by flash chromatography (dichloromethane to 94:6 dichloromethane/methanol) to yield 98 mg (16%) of the minor isomer, first peak to elute) and 125 mg (21%) of the major isomer, second peak to elute, as solids.

Minor Isomer:

LRMS (m/z): 495 (M+1)⁺

¹H NMR [symbol] (CDCl₃): 1.4 (s, 9H), 1.6-2.1 (m, 5H), 3.4-3.6 (m, 3H), 3.8 (d, 1H), 4.6 (s, 3H), 5.7 (bs, 1H), 7.3 (m, 1H), 7.7 (dd, 1H), 8.5 (s, 1H), 9.8 (dd, 1H).

Major Isomer:

LRMS (m/z): 495 (M+1)⁺

¹H NMR [symbol] (CDCl₃): 1.4 (s, 9H), 1.6-2.1 (m, 5H), 3.5 (m, 3H), 3.8 (d, 1H), 4.5 (s, 3H), 5.3 (bs, 1H), 7.1 (s, 1H), 7.3 (m, 1H), 7.7 (dd, 1H), 8.6 (s, 1H), 10.1 (dd, 1H).

### Preparation 90

### (R)-2-(6-Fluoroimidazo[1,2-a]pyridin-3-yl)-6-(1-methyl-1H-tetrazol-5-yl)-N-(piperidin-3-yl)pyrimidin-4-amine

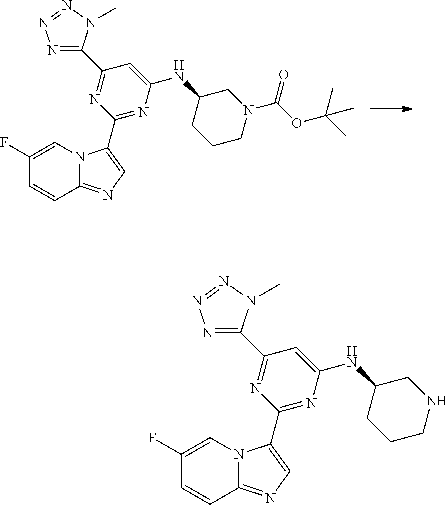 [SMILES: Cn1nnnc1-c1cc(N[C@@H]2CCCN(C(=O)OC(C)(C)C)C2)nc(-c2cnc3ccc(F)cn23)n1 ; Cn1nnnc1-c1cc(N[C@@H]2CCCNC2)nc(-c2cnc3ccc(F)cn23)n1]

Prepared from (R)-tert-butyl 3-(2-(6-fluoroimidazo[1,2-a]pyridin-3-yl)-6-(1-methyl-1H-tetrazol-5-yl)pyrimidin-4-ylamino)piperidine-1-carboxylate (98 mg, 0.20 mmol, Minor isomer from Preparation 89) and trifluoroacetic acid (152 μL, 1.97 mmol) following the procedure described in Preparation 74b. 48 mg (61%) of the title compound were obtained and used in the next step without further purification.

LRMS (m/z): 395 (M+1)⁺

¹H NMR [symbol] (CDCl₃): 1.6-2.0 (m, 5H), 2.9 (m, 3H), 3.2 (dd, 1H), 4.3 (bs, 1H), 4.6 (s, 3H), 6.0 (bs, 1H), 7.2 (s, 1H), 7.3 (m, 1H), 7.7 (dd, 1H), 8.5 (s, 1H), 9.8 (dd, 1H).

### Preparation 91

### (R)-2-(6-Fluoroimidazo[1,2-a]pyridin-3-yl)-6-(2-methyl-2H-tetrazol-5-yl)-N-(piperidin-3-yl)pyrimidin-4-amine

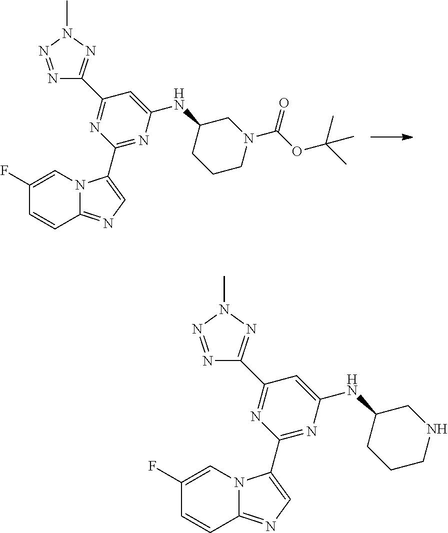 [SMILES: Cn1nnc(-c2cc(N[C@@H]3CCCN(C(=O)OC(C)(C)C)C3)nc(-c3cnc4ccc(F)cn34)n2)n1 ; Cn1nnc(-c2cc(N[C@@H]3CCCNC3)nc(-c3cnc4ccc(F)cn34)n2)n1]

Prepared from (R)-tert-butyl 3-(2-(6-fluoroimidazo[1,2-a]pyridin-3-yl)-6-(2-methyl-2H-tetrazol-5-yl)pyrimidin-4-ylamino)piperidine-1-carboxylate (125 mg, 0.25 mmol, Major isomer from Preparation 89) and trifluoroacetic acid (195 μL, 2.53 mmol) following the procedure described in Preparation 74b. 40 mg (40%) of the title compound were obtained and used in the next step without further purification.

LRMS (m/z): 395 (M+1)⁺

¹H NMR [symbol] (CDCl₃): 1.6-2.0 (m, 5H), 2.8-2.9 (m, 3H), 3.2 (dd, 1H), 4.5 (s, 3H), 5.7 (bs, 1H), 7.1 (s, 1H), 7.3 (m, 1H), 7.7 (dd, 1H), 8.6 (s, 1H), 10.1 (dd, 1H).

### Preparation 92

### (R)-2-(6-Fluoroimidazo[1,2-a]pyridin-3-yl)-6-(piperidin-3-ylamino)pyrimidine-4-carboxylic acid

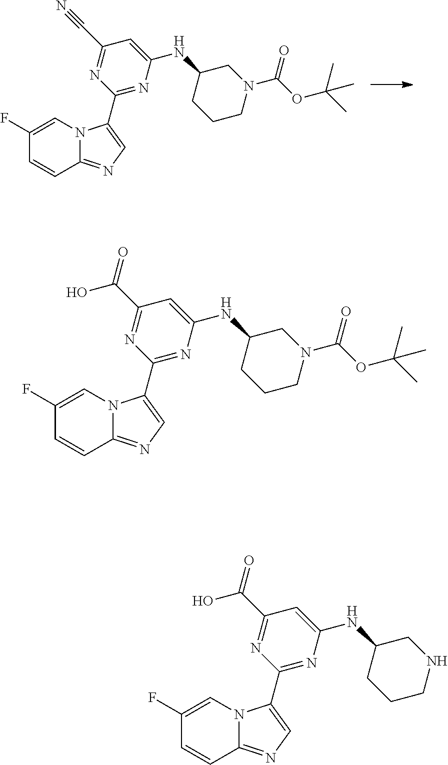 [SMILES: CC(C)(C)OC(=O)N1CCC[C@@H](Nc2cc(C#N)nc(-c3cnc4ccc(F)cn34)n2)C1 ; CC(C)(C)OC(=O)N1CCC[C@@H](Nc2cc(C(=O)O)nc(-c3cnc4ccc(F)cn34)n2)C1 ; O=C(O)c1cc(N[C@@H]2CCCNC2)nc(-c2cnc3ccc(F)cn23)n1]

### a) (R)-6-(1-(tert-Butoxycarbonyl)piperidin-3-ylamino)-2-(6-fluoroimidazo[1,2-a]pyridin-3-yl)pyrimidine-4-carboxylic acid

A 8N aqueous solution of sodium hydroxide (4.5 mL, 36 mmol) was added to a solution of (R)-tert-butyl 3-(6-cyano-2-(6-fluoroimidazo[1,2-a]pyridin-3-yl)pyrimidin-4-ylamino) piperidine-1-carboxylate (450 mg, 1.03 mmol, Preparation 87a) in methanol (8 mL) and the resulting mixture was heated at 100° C. for 1 hour under microwave irradiation. The reaction mixture was then diluted with water and after washing the aqueous solution with chloroform, it was acidified until a white solid precipitated (pH=4). The product was extracted with chloroform and the organic phase was washed with water and brine, dried over magnesium sulphate—and the solvent evaporated to yield 247 mg (53%) of the title product as a white solid.

LRMS (m/z): 457 (M+1)⁺

### b) (R)-2-(6-Fluoroimidazo[1,2-a]pyridin-3-yl)-6-(piperidin-3-ylamino)pyrimidine-4-carboxylic acid

Prepared from (R)-6-(1-(tert-butoxycarbonyl)piperidin-3-ylamino)-2-(6-fluoroimidazo[1,2-a]pyridin-3-yl)pyrimidine-4-carboxylic acid (247 mg, 0.54 mmol, Preparation 92a) and trifluoroacetic acid (210 μL, 2.73 mmol) following the procedure described in Preparation 74b. 167 mg (87%) of the title compound were obtained and used in the next step without further purification.

LRMS (m/z): 357 (M+1)⁺

### Preparation 93

### (R)-2-(6-Fluoroimidazo[1,2-a]pyridin-3-yl)-N⁴-(piperidin-3-yl)pyrimidine-4,6-diamine

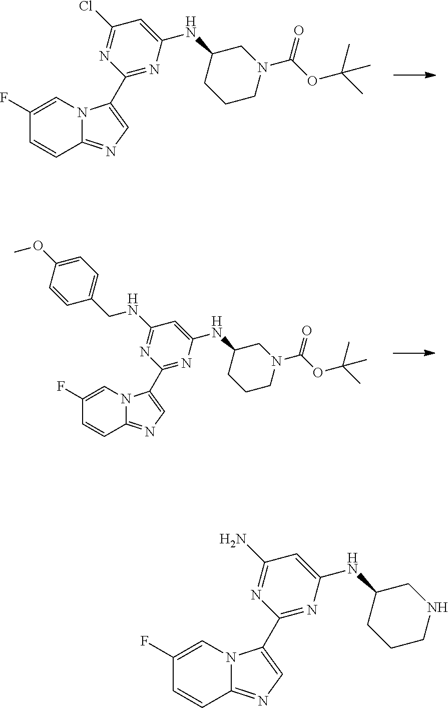

### a) (R)-tert-Butyl 3-(2-(6-fluoroimidazo[1,2-a]pyridin-3-yl)-6-(4-methoxy benzylamino)pyrimidin-4-ylamino)piperidine-1-carboxylate

Prepared from (R)-tert-butyl 3-(6-chloro-2-(6-fluoroimidazo[1,2-a]pyridin-3-yl)pyrimidin-4-ylamino)piperidine-1-carboxylate (245 mg, 0.55 mmol, Preparation 73) and (4-methoxyphenyl)methanamine (376 mg, 2.74 mmol) following the procedure described in Preparation 76a. The crude product was purified by flash chromatography (dichloromethane to 93:7 dichloromethane/methanol) to yield 181 mg (60%) of the title product as a white solid.

LRMS (m/z): 548 (M+1)⁺

¹H NMR [symbol] (CDCl₃): 1.4 (s, 9H), 1.6-2.0 (m, 3H), 3.2 (bs, 2H), 3.6 (m, 3H), 3.8 (s, 3H), 4.5 (d, 2H), 4.7 (d, 1H), 5.1 (bs, 1H), 5.3 (bs, 1H), 6.9 (d, 2H), 7.2 (m, 1H), 7.3 (d, 2H), 7.6 (dd, 1H), 8.5 (s, 1H), 9.9 (dd, 1H).

### b) (R)-2-(6-Fluoroimidazo[1,2-a]pyridin-3-yl)-N⁴-(piperidin-3-yl)pyrimidine-4,6-diamine

Trifluoroacetic acid (255 μL, 3.32 mmol) was added to a solution of (R)-tert-butyl 3-(2-(6-fluoroimidazo[1,2-a]pyridin-3-yl)-6-(4-methoxybenzylamino)pyrimidin-4-ylamino) piperidine-1-carboxylate (181 mg, 0.33 mmol, Preparation 93a) in methylene chloride (2 mL) and the reaction mixture was stirred at room temperature for 64 hours and at 50° C. for additional 20 hours. The solvent was then evaporated and the residue was treated with a saturated aqueous solution of sodium hydrogencarbonate. The aqueous solution was extracted with chloroform and the organic layer was washed with water and brine, dried over magnesium sulphate and the solvent evaporated to give 108 mg (100%) of the title product as a yellowish solid.

LRMS (m/z): 328 (M+1)⁺

¹H NMR [symbol] (CDCl₃): 1.8-2.0 (m, 3H), 2.6-2.9 (m, 4H), 3.2 (dd, 1H), 4.6 (bs, 2H), 5.1 (d, 1H), 5.4 (s, 1H), 7.2 (m, 1H), 7.6 (dd, 1H), 8.5 (s, 1H), 10.0 (dd, 1H).

### Preparation 94

### 3-(4,6-Dichloro-5-fluoropyrimidin-2-yl)-6-fluoroimidazo[1,2-a]pyridine

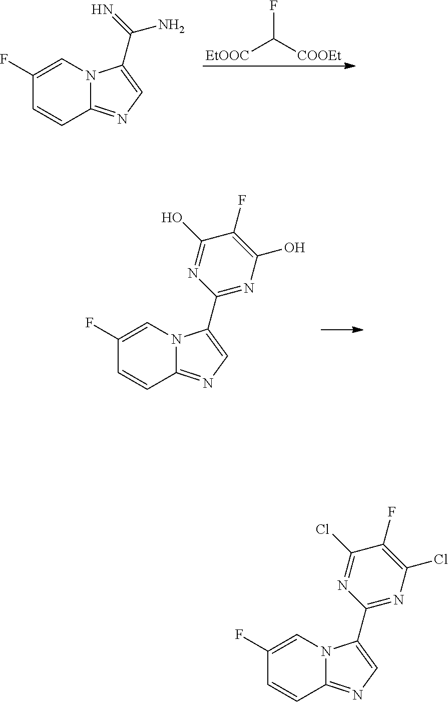 [SMILES: N=C(N)c1cnc2ccc(F)cn12 ; CCOC(=O)C(F)C(=O)OCC ; Oc1nc(-c2cnc3ccc(F)cn23)nc(O)c1F ; Fc1ccc2ncc(-c3nc(Cl)c(F)c(Cl)n3)n2c1]

### a) 5-Fluoro-2-(6-fluoroimidazo[1,2-a]pyridin-3-yl)pyrimidine-4,6-diol

Prepared from 6-fluoroimidazo[1,2-a]pyridine-3-carboximidamide (2.00 g, 8.39 mmol, Preparation 42b) and diethyl 2-fluoromalonate (2.65 mL, 16.8 mmol) following the procedure described in Preparation 72a. 0.95 g (43%) of the title product were isolated as a white solid.

LRMS (m/z): 265 (M+1)⁺

¹H NMR [symbol] (DMSO-d⁶): 7.6 (s, 1H), 7.8 (s, 1H), 8.7 (s, 1H), 10.1 (s, 1H), 12.6 (bs, 1H).

### b) 3-(4,6-Dichloro-5-fluoropyrimidin-2-yl)-6-fluoroimidazo[1,2-a]pyridine

Prepared from 5-fluoro-2-(6-fluoroimidazo[1,2-a]pyridin-3-yl)pyrimidine-4,6-diol (0.95 g, 3.60 mmol, Preparation 94a) and phosphorus oxychloride (7.35 mL) following the procedure described in Preparation 72b. The title compound was obtained as a solid (0.71 g, 63%) which was used in the next step without further purification.

LRMS (m/z): 302 (M+1)⁺

### Preparation 95

### (R)-5-Fluoro-2-(6-fluoroimidazo[1,2-a]pyridin-3-yl)-6-morpholino-N-(piperidin-3-yl)pyrimidin-4-amine

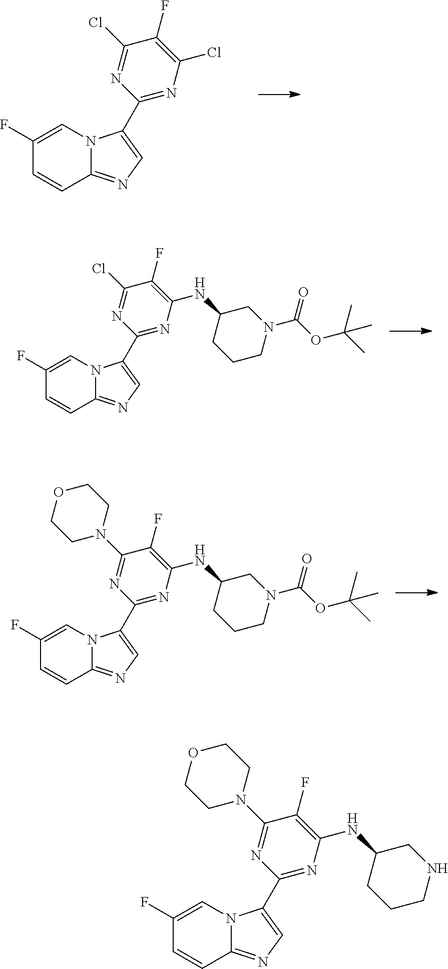 [SMILES: Fc1ccc2ncc(-c3nc(Cl)c(F)c(Cl)n3)n2c1 ; CC(C)(C)OC(=O)N1CCC[C@@H](Nc2nc(-c3cnc4ccc(F)cn34)nc(Cl)c2F)C1 ; CC(C)(C)OC(=O)N1CCC[C@@H](Nc2nc(-c3cnc4ccc(F)cn34)nc(N3CCOCC3)c2F)C1 ; Fc1ccc2ncc(-c3nc(N[C@@H]4CCCNC4)c(F)c(N4CCOCC4)n3)n2c1]

### a) (R)-tert-Butyl 3-(6-chloro-5-fluoro-2-(6-fluoroimidazo[1,2-a]pyridin-3-yl)pyrimidin-4-ylamino)piperidine-1-carboxylate

Prepared from 3-(4,6-dichloro-5-fluoropyrimidin-2-yl)-6-fluoroimidazo[1,2-a]pyridine (420 mg, 1.39 mmol, Preparation 94b) and (R)-tert-butyl 3-aminopiperidine-1-carboxylate (560 mg, 2.79 mmol) following the procedure described in Preparation 73. The crude product was purified by flash chromatography (dichloromethane to 95:5 dichloromethane/methanol) to yield 400 mg (62%) of the title compound.

LRMS (m/z): 465 (M+1)⁺

¹H NMR [symbol] (CDCl₃): 1.4 (s, 9H), 1.6-2.1 (m, 4H), 3.5 (m, 3H), 3.8 (d, 1H), 4.2 (m, 1H), 5.4 (bs, 1H), 7.3 (m, 1H), 7.7 (dd, 1H), 8.5 (s, 1H), 9.7 (dd, 1H).

### b) (R)-tert-Butyl 3-(5-fluoro-2-(6-fluoroimidazo[1,2-a]pyridin-3-yl)-6-morpholino pyrimidin-4-ylamino)piperidine-1-carboxylate

Prepared from (R)-tert-butyl 3-(6-chloro-5-fluoro-2-(6-fluoroimidazo[1,2-a]pyridin-3-yl)pyrimidin-4-ylamino)piperidine-1-carboxylate (190 mg, 0.41 mmol, Preparation 95a) and morpholine (180 mg, 2.07 mmol) in N-methylpirrolidone (2 mL) following the procedure described in Preparation 76a. The mixture was heated under microwave irradiation at 130° C. for 2 hours. 140 mg (66%) of the title compound were obtained and used in the next step without further purification

LRMS (m/z): 516 (M+1)⁺

¹H NMR [symbol] (CDCl₃): 1.4 (s, 9H), 1.6-1.9 (m, 5H), 3.5 (bs, 3H), 3.7 (m, 4H), 3.8 (m, 4H), 4.2 (m, 1H), 4.9 (bs, 1H), 7.2 (m, 1H), 7.7 (dd, 1H), 8.4 (s, 1H), 9.8 (dd, 1H).

### c) (R)-5-Fluoro-2-(6-fluoroimidazo[1,2-a]pyridin-3-yl)-6-morpholino-N-(piperidin-3-yl)pyrimidin-4-amine

Prepared from (R)-tert-butyl 3-(5-fluoro-2-(6-fluoroimidazo[1,2-a]pyridin-3-yl)-6-morpholinopyrimidin-4-ylamino)piperidine-1-carboxylate (140 mg, 0.27 mmol, Preparation 95b) and trifluoroacetic acid (50 μL, 0.65 mmol) following the procedure described in Preparation 74b. 80 mg (71%) of the title compound were obtained as a solid and used in the next step without further purification.

LRMS (m/z): 416 (M+1)⁺

### Preparation 96

### 6-Fluoro-3-(4-fluoro-6-methoxy-5-(trifluoromethyl)pyrimidin-2-yl)imidazo[1,2-a]pyridine

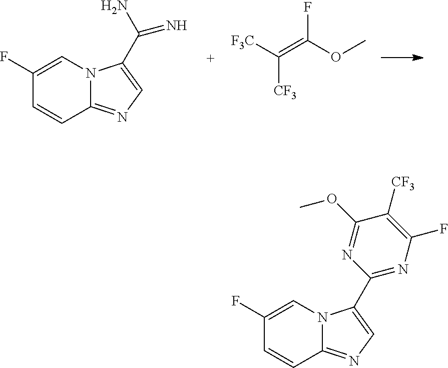 [SMILES: N=C(N)c1cnc2ccc(F)cn12 ; COC(F)=C(C(F)(F)F)C(F)(F)F ; COc1nc(-c2cnc3ccc(F)cn23)nc(F)c1C(F)(F)F]

Water (8 mL) and 1,3,3,3-tetrafluoro-1-methoxy-2-(trifluoromethyl)prop-1-ene (670 μL, 7.08 mmol) were added to a suspension of 6-fluoroimidazo[1,2-a]pyridine-3-carboximidamide (1.52 g, 4.76 mmol, Preparation 42b) in methylene chloride (10 mL). The reaction mixture was cooled in an ice bath and a 8M aqueous solution of sodium hydroxide (2.36 mL, 19 mmol) was added. After stirring for 3 hours at ambient temperature, the mixture was diluted with excess of methylene chloride. The organic layer was separated and washed with aqueous 2N hydrochloric acid solution, water and brine, dried over magnesium sulphate and the solvent evaporated to give 236 mg (15%) of the title compound as a brownish solid, which was used without further purification in the next step.

LRMS (m/z): 331 (M+1)⁺

### Preparation 97

### (R)-2-(6-Fluoroimidazo[1,2-a]pyridin-3-yl)-6-methoxy-N-(piperidin-3-yl)-5-(trifluoromethyl)pyrimidin-4-amine

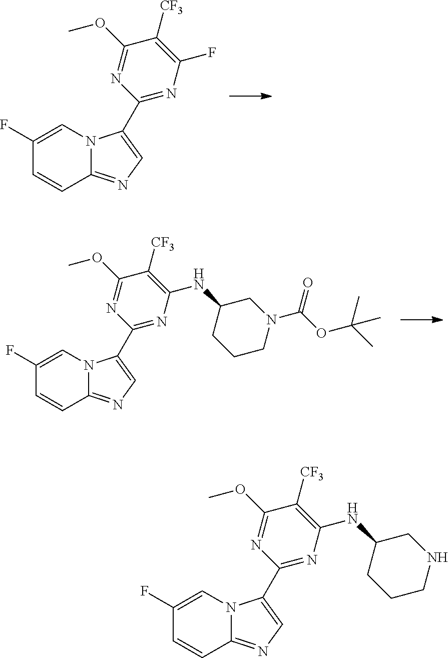 [SMILES: COc1nc(-c2cnc3ccc(F)cn23)nc(F)c1C(F)(F)F ; COc1nc(-c2cnc3ccc(F)cn23)nc(N[C@@H]2CCCN(C(=O)OC(C)(C)C)C2)c1C(F)(F)F ; COc1nc(-c2cnc3ccc(F)cn23)nc(N[C@@H]2CCCNC2)c1C(F)(F)F]

### a) (R)-tert-Butyl 3-(2-(6-fluoroimidazo[1,2-a]pyridin-3-yl)-6-methoxy-5-(trifluoromethyl)pyrimidin-4-ylamino)piperidine-1-carboxylate

(R)-tert-butyl 3-aminopiperidine-1-carboxylate (286 mg, 1.43 mmol) was added to a solution of 6-fluoro-3-(4-fluoro-6-methoxy-5-(trifluoromethyl)pyrimidin-2-yl)imidazo[1,2-a]pyridine (236 mg, 0.71 mmol, Preparation 96) in ethanol (5 mL) and the resulting mixture was stirred at room temperature for 3 hours. The solid that precipitated was filtered, washed and dried. The solvent of the filtrates was evaporated and the residue was purified by flash chromatography (dichloromethane to 95:5 dichloromethane/methanol). The solid obtained through filtration and the one coming from the purification were combined to yield 283 mg (78%) of the title compound.

LRMS (m/z): 511 (M+1)⁺

### b) (R)-2-(6-Fluoroimidazo[1,2-a]pyridin-3-yl)-6-methoxy-N-(piperidin-3-yl)-5-(trifluoromethyl)pyrimidin-4-amine

Prepared from (R)-tert-butyl 3-(2-(6-fluoroimidazo[1,2-a]pyridin-3-yl)-6-methoxy-5-(trifluoromethyl)pyrimidin-4-ylamino)piperidine-1-carboxylate (106 mg, 0.21 mmol, Preparation 97a) and trifluoroacetic acid (80 μL, 1.04 mmol) following the procedure described in Preparation 74b. 81 mg (95%) of the title compound were obtained as a white solid.

LRMS (m/z): 411 (M+1)⁺

¹H NMR [symbol] (CDCl₃): 1.6-2.0 (m, 5H), 2.8-2.9 (m, 3H), 3.2 (dd, 1H), 4.1 (s, 3H), 4.3 (m, 1H), 6.2 (bs, 1H), 7.3 (m, 1H), 7.7 (dd, 1H), 8.6 (s, 1H), 9.8 (dd, 1H).

### EXAMPLE 1

### 3-(4-{[(1S)-1-Phenylethyl]amino}pyrimidin-2-yl)imidazo[1,2-a]pyridine-6-carbonitrile

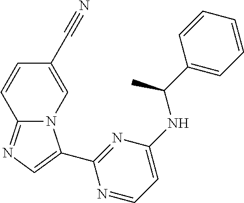 [SMILES: C[C@H](Nc1ccnc(-c2cnc3ccc(C#N)cn23)n1)c1ccccc1]

Obtained as a yellow solid (0.102 g, 40%) from 3-(4-hydroxypyrimidin-2-yl)imidazo[1,2-a]pyridine-6-carbonitrile (Preparation 4b, 0.175 g, 0.74 mmol) and (S)-1-phenyl-ethylamine (0.48 mL, 3.68 mmol) following the experimental procedure as described in Preparation 5a. The solvent was removed in vacuum and the residue was purified by reverse phase chromatography (C-18 silica from Waters, water/1:1 acetonitrile-methanol as eluents [0.1% v/v formic acid buffered] 0% to 100%) to give the title compound (0.102 g, 40%) as a yellow solid.

LRMS (m/z): 341 (M+1)⁺.

¹H-NMR δ (DMSO-d₆): 1.53 (d, 3H), 5.28 (bs, 1H), 6.52 (bs, 1H), 7.22 (d, 1H), 7.36 (d, 2H), 7.48 (d, 2H), 7.67 (d, 1H), 7.87 (d, 1H), 8.19 (d, 1H), 8.26 (bs, 1H), 8.42 (bs, 1H), 10.27 (bs, 1H).

### EXAMPLE 2

### 3-(4-{[(1R)-1-Phenylethyl]amino}pyrimidin-2-yl)imidazo[1,2-a]pyridine-6-carbonitrile

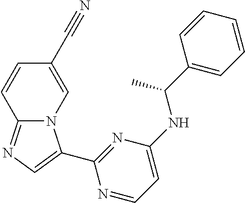 [SMILES: C[C@@H](Nc1ccnc(-c2cnc3ccc(C#N)cn23)n1)c1ccccc1]

Obtained as a white solid (70%) from 3-(4-hydroxypyrimidin-2-yl)imidazo[1,2-a]pyridine-6-carbonitrile (Preparation 4b) and (R)-1-phenyl-ethylamine following the experimental procedure as described in Preparation 5a. After complete reaction, the mixture was partitioned between water and dichloromethane. The organic layer was washed with water, brine, dried (MgSO₄) and evaporated and the residue was purified by flash chromatography (98:2 to 96:4 dichloromethane/methanol).

LRMS (m/z): 341 (M+1)⁺.

¹H-NMR δ (DMSO-d₆): 1.52 (d, 3H), 5.30 (bs, 1H), 6.52 (d, 1H), 7.22 (d, 1H), 7.34 (t, 2H), 7.48 (d, 2H), 7.68 (d, 1H), 7.88 (d, 1H), 8.19 (d, 1H), 8.24 (d, 1H), 8.43 (s, 1H), 10.28 (bs, 1H)

### EXAMPLE 3

### 3-[4-(Benzylamino)pyrimidin-2-yl]imidazo[1,2-a]pyridine-6-carbonitrile

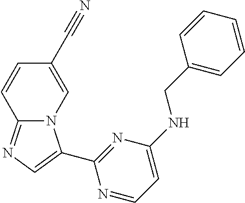 [SMILES: N#Cc1ccc2ncc(-c3nccc(NCc4ccccc4)n3)n2c1]

Obtained as a yellow solid (60%) from 3-(4-hydroxypyrimidin-2-yl)imidazo[1,2-a]pyridine-6-carbonitrile (Preparation 4b) and phenylmethanamine following the experimental procedure as described in Preparation 5a. The desired compound was obtained after purification of the reaction crude by reverse phase chromatography (C-18 silica from Waters, water/1:1 acetonitrile-methanol as eluents [0.1% v/v formic acid buffered] 0% to 100%).

LRMS (m/z): 327 (M+1)⁺.

¹H-NMR δ (DMSO-dₑ): 5.76 (bs, 2H), 6.61 (bs, 1H), 7.25-7.4 (m, 5H), 7.68 (d, 1H), 7.89 (d, 1H), 8.17-8.36 (m, 2H), 8.48 (s, 1H), 10.37 (bs, 1H)

### EXAMPLE 4

### 3-(4-{[(1S)-2-Methoxy-1-methylethyl]amino}pyrimidin-2-yl)imidazo[1,2-a]pyridine-6-carbonitrile

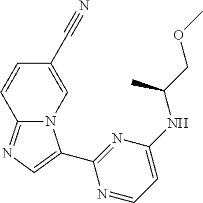 [SMILES: COC[C@H](C)Nc1ccnc(-c2cnc3ccc(C#N)cn23)n1]

Obtained as a white solid (33%) from 3-(4-hydroxypyrimidin-2-yl)imidazo[1,2-a]pyridine-6-carbonitrile (Preparation 4b) and (S)-1-methoxypropan-2-amine following the experimental procedure as described in Preparation 5a. After complete reaction the mixture was partitioned between water and dichloromethane. The organic layer was washed with water, brine, dried (MgSO₄) and evaporated and the residue was purified by flash chromatography (98:2 to 96:4 dichloromethane/methanol).

LRMS (m/z): 307 (M−1)⁺.

¹H-NMR δ (CDCl₃): 1.36 (d, 3H), 3.43 (s, 3H), 4.27 (bs, 1H), 5.25 (bs, 1H), 6.26 (d, 1H), 7.40 (d, 1H), 7.77 (d, 1H), 8.24 (d, 1H), 8.58 (s, 1H), 10.56 (s, 1H)

### EXAMPLE 5

### 3-{4-[(Cyclohexylmethyl)amino]pyrimidin-2-yl}imidazo[1,2-a]pyridine-6-carbonitrile

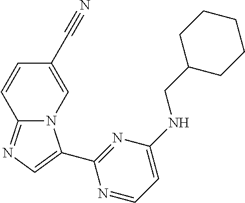 [SMILES: N#Cc1ccc2ncc(-c3nccc(NCC4CCCCC4)n3)n2c1]

Obtained as a white solid (50%) from 3-(4-hydroxypyrimidin-2-yl)imidazo[1,2-a]pyridine-6-carbonitrile (Preparation 4b) and cyclohexylmethanamine following the experimental procedure as described in Preparation 5a. After complete reaction, the mixture was partitioned between water and dichloromethane. The organic layer was washed with water and then extracted with 2M aqueous hydrochloric acid. The aqueous phase was basified with 6M aqueous sodium hydroxide solution and then extracted with dichloromethane. The organic layer was dried (MgSO₄) and evaporated to give the title compound.

LRMS (m/z): 333 (M+1)⁺.

¹H-NMR δ (CDCl₃): 0.91-1.93 (m, 10H), 1.92-2.05 (m, 1H), 3.13-3.33 (bs, 1H), 3.40 (d, 2H), 6.24 (d, 1H), 7.40 (d, 1H), 7.78 (d, 1H), 8.18-8.36 (m, 1H), 8.59 (bs, 1H), 10.56 (bs, 1H).

### EXAMPLE 6

### 3-{4[(2-Methoxyethyl)amino]pyrimidin-2-yl}imidazo[1,2-a]pyridine-6-carbonitrile

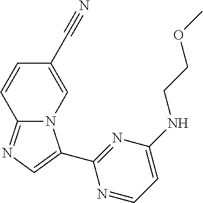 [SMILES: COCCNc1ccnc(-c2cnc3ccc(C#N)cn23)n1]

Obtained as a white solid (60%) from 3-(4-hydroxypyrimidin-2-yl)imidazo[1,2-a]pyridine-6-carbonitrile (Preparation 4b) and 2-methoxyethanamine following the experimental procedure as described in Preparation 5a.

LRMS (m/z): 295 (M+1)⁺.

¹H-NMR δ (DMSO-d₆): 3.08-3.76 (m, 4H), 3.36 (s, 3H), 6.50 (d, 1H), 7.73 (d, 1H), 7.84 (bs, 1H), 7.94 (d, 1H), 8.23 (bs, 1H), 8.52 (s, 1H), 10.52 (bs, 1H)

### EXAMPLE 7

### 3-{4-[(1-Adamantylmethyl)amino]pyrimidin-2-yl}imidazo[1,2-a]pyridine-6-carbonitrile

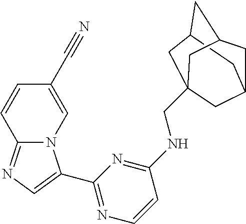 [SMILES: N#Cc1ccc2ncc(-c3nccc(NCC45CC6CC(CC(C6)C4)C5)n3)n2c1]

Obtained as a white solid (51%) from 3-(4-hydroxypyrimidin-2-yl)imidazo[1,2-a]pyridine-6-carbonitrile (Preparation 4b) and 1-(1-adamantyl)methanamine following the experimental procedure as described in Preparation 5a. The crude product was purified by flash chromatography (100:8:1 dichloromethane/methanol/aqueous ammonia solution).

LRMS (m/z): 385 (M+1)⁺.

¹H-NMR δ (DMSO-d₆): 1.60-1.66 (m, 12H), 1.86-2.03 (m, 3H), 4.67 (s, 2H), 6.48 (d, 1H), 7.64 (bs, 1H), 7.68 (d, 1H), 7.89 (d, 1H), 8.13 (d, 1H), 8.48 (bs, H) 10.56 (bs, 1H).

### EXAMPLE 8

### 3-{4-[(2,2-Dimethylpropyl)amino]pyrimidin-2-yl}imidazo[1,2-a]pyridine-6-carbonitrile

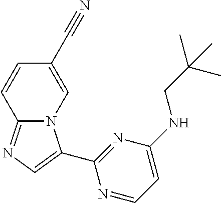 [SMILES: CC(C)(C)CNc1ccnc(-c2cnc3ccc(C#N)cn23)n1]

Obtained as a white solid (26%) from 3-(4-hydroxypyrimidin-2-yl)imidazo[1,2-a]pyridine-6-carbonitrile (Preparation 4b) and 2,2-dimethylpropan-1-amine following the experimental procedure as described in Preparation 5a. The crude product was purified by reverse phase chromatography (C-18 silica from Waters, water/1:1 acetonitrile-methanol as eluents [0.1% v/v formic acid buffered] 0% to 100%).

LRMS (m/z): 307 (M+1)⁺.

¹H-NMR δ (DMSO-d₆): 1.03 (s, 9H), 4.17 (d, 2H), 6.54 (d, 1H), 7.64-7.73 (bs, 1H), 7.72 (d, 1H), 7.95 (d, 1H), 8.19 (d, 1H), 8.53 (s, 1H), 10.59 (bs, 1H).

### EXAMPLE 9

### 3-{5-Bromo-4-[(2,2-dimethylpropyl)amino]pyrimidin-2-yl}imidazo[1,2-a]pyridine-6-carbonitrile

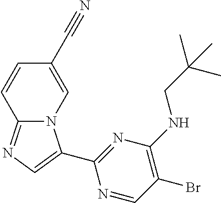 [SMILES: CC(C)(C)CNc1nc(-c2cnc3ccc(C#N)cn23)ncc1Br]

To a stirred solution of 3-{4-[(2,2-dimethylpropyl)amino]pyrimidin-2-yl}imidazo[1,2-a]pyridine-6-carbonitrile (Example 8, 0.300 g, 1.0 mmol) in acetic acid (3.6 mL) was added potassium acetate (0.104 g, 1.1 mmol). The mixture was cooled (ice-bath) and a solution of bromine (0.050 mL, 1.0 mmol) in acetic acid (0.2 mL) was added dropwise. The reaction was then stirred at ambient temperature for 1 hour. Saturated aqueous sodium carbonate solution (30 mL) was added and the mixture was extracted with ethyl acetate. The organic layer was washed with brine, dried (MgSO₄) and evaporated to give the title compound (0.390 g, 98%) as an off-white solid.

LRMS (m/z): 385/387 (M+1)⁺.

¹H-NMR δ (CDCl₃): 1.06 (s, 9H), 3.49 (d, 2H), 5.62 (bt, 1H), 7.42 (ddd, 1H), 7.80 (ddd, 2H), 8.37 (d, 1H), 8.60 (d, 1H), 10.48 (m, 1H)

### EXAMPLE 10

### 3-{4-[(2,2-Dimethylpropyl)amino]-5-piperazin-1-ylpyrimidin-2-yl}imidazo[1,2-a]pyridine-6-carbonitrile

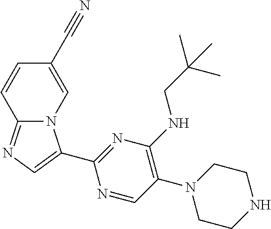 [SMILES: CC(C)(C)CNc1nc(-c2cnc3ccc(C#N)cn23)ncc1N1CCNCC1]

a) Nitrogen was bubbled for 5 minutes through a mixture 3-{5-bromo-4-[(2,2-dimethyl propyl)amino]pyrimidin-2-yl}imidazo[1,2-a]pyridine-6-carbonitrile (Example 9, 0.105 g, 0.27 mmol), tert-butyl piperazine-1-carboxylate (0.152 g, 0.82 mmol), sodium tert-butoxide (0.079 g, 0.82 mmol) and biphenyl-2-yl-di-tert-butylphosphine (0.020 g, 0.07 mmol) in toluene (2.5 mL) contained in a microwave reaction vessel. Tris(dibenzylideneacetone)dipalladium (0) (0.125 g, 0.14 mmol) was then added and the vessel was sealed and subjected to microwave irradiation for 1 hour at 115° C. Water and ethyl acetate were added to the mixture and the organic layer was dried (MgSO₄) and evaporated. Purification of the residue by reverse phase chromatography (C-18 silica from Waters, water/1:1 acetonitrile-methanol as eluents [0.1% v/v formic acid buffered] 0% to 100%) gave tert-butyl-4-{2-(6-cyanoimidazo[1,2-a]pyridin-3-yl)-4-[(2,2-dimethylpropyl)amino]pyrimidin-5-yl}piperazine-1-carboxylate (0.023 g, 16%) as a yellow solid.

LRMS (m/z): 491 (M+1)⁺.

¹H-NMR δ (CDCl₃): 1.05 (s, 9H), 1.52 (d, 9H), 2.18 (d, 2H), 3.00-2.86 (m, 4H), 3.53-3.39 (m, 4H), 7.38 (d, 1H), 7.77 (d, 1H), 8.00 (s, 1H), 8.55 (s, 1H), 10.60 (s, 1H).

b) To a solution of tert-butyl 4-{2-(6-cyanoimidazo[1,2-a]pyridin-3-yl)-4-[(2,2-dimethyl propyl)amino]pyrimidin-5-yl}piperazine-1-carboxylate (0.022 g, 0.04 mmol) in dioxane (1 mL) was added a 4M solution of hydrogen chloride in 1,4-dioxane (0.140 mL, 0.56 mmol). The mixture was stirred at ambient temperature for 20 hours and then solvent was evaporated and water was added. The aqueous layer was washed with dichloromethane and then basified with concentrated aqueous ammonia solution and extracted with dichloromethane. The organic layer was dried (MgSO₄) and evaporated to obtain (0.008 g, 41%) of the title compound as a pale yellow solid.

LRMS (m/z): 391 (M+1)⁺.

¹H-NMR δ (CDCl₃): 1.06 (s, 9H), 3.13-2.90 (m, 8H), 3.46-3.40 (m, 2H), 5.91 (t, 1H), 7.40-7.35 (m, 1H), 7.77 (d, 1H), 8.02 (s, 1H), 8.54 (s, 1H), 10.61 (s, 1H).

### EXAMPLE 11

### 3-[4-[(2,2-Dimethylpropyl)amino]-5-(2-methoxypyridin-4-yl)pyrimidin-2-yl]imidazo[1,2-a]pyridine-6-carbonitrile

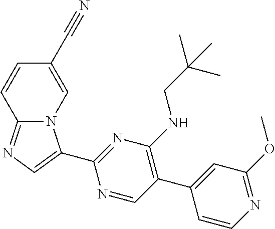 [SMILES: COc1cc(-c2cnc(-c3cnc4ccc(C#N)cn34)nc2NCC(C)(C)C)ccn1]

Obtained as a pale yellow solid (54%) from 3-{5-bromo-4-[(2,2-dimethylpropyl)amino]pyrimidin-2-yl}imidazo[1,2-a]pyridine-6-carbonitrile (Example 9) and 2-methoxy-4-(4,4,5,5-tetramethyl-1,3,2-dioxaborolan-2-yl)pyridine (Preparation 38) following the experimental procedure as described in Preparation 27d. The crude product mixture was filtered through Celite® washing the filter cake with ethyl acetate. The solvent was removed under reduced pressure and the residue was purified by reverse phase chromatography (C-18 silica from Waters, water/1:1 acetonitrile-methanol as eluents [0.1% v/v formic acid buffered] 0% to 100%) to yield the desired compound.

LRMS (m/z): 414 (M+1)⁺.

¹H-NMR δ (CDCl₃): 1.03 (s, 9H), 3.47 (bs, 2H), 4.04 (s, 3H), 5.31 (d, 1H), 6.86 (s, 1H), 6.99 (t, 1H), 7.44 (d, 1H), 7.82 (d, 1H), 8.18 (s, 1H), 8.34 (t, 1H), 8.67 (s, 1H), 10.62 (s, 1H).

### EXAMPLE 12

### 3-[4-[(2,2-Dimethylpropyl)amino]-5-(2-oxo-1,2-dihydropyridin-4-yl)pyrimidin-2-yl]imidazo[1,2-a]pyridine-6-carbonitrile

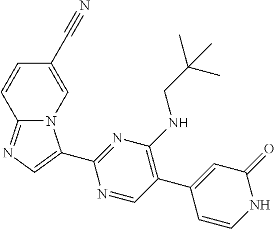 [SMILES: CC(C)(C)CNc1nc(-c2cnc3ccc(C#N)cn23)ncc1-c1cc[nH]c(=O)c1]

Obtained as solid (63%) from 3-[4-[(2,2-dimethylpropyl)amino]-5-(2-methoxypyridin-4-yl)pyrimidin-2-yl]imidazo[1,2-a]pyridine-6-carbonitrile (Example 11) following the experimental procedure as described in Preparation 4b.

LRMS (m/z): 400 (M+1)⁺.

¹H-NMR δ (DMSO-d₆): 1.05 (s, 9H), 3.47 (d, 2H), 6.34 (d, 1H), 6.53 (s, 1H), 7.45 (bs, 1H), 7.60 (d, 1H), 7.87 (d, 1H), 8.05 (d, 1H), 8.28 (s, 1H), 8.72 (s, 1H), 10.54 (s, 1H).

### EXAMPLE 13

### 3-{4-[(2,2-Dimethylpropyl)amino]-5-pyridin-3-ylpyrimidin-2-yl}imidazo[1,2-a]pyridine-6-carbonitrile

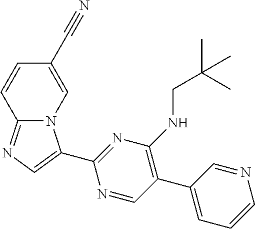 [SMILES: CC(C)(C)CNc1nc(-c2cnc3ccc(C#N)cn23)ncc1-c1cccnc1]

Obtained as a yellow solid (22%) from 3-(4-hydroxy-5-pyridin-3-ylpyrimidin-2-yl)imidazo[1,2-a]pyridine-6-carbonitrile (Preparation 26b) and neopentylamine following the experimental procedure as described in Preparation 5a followed by purification of the crude product by reverse phase chromatography (C-18 silica from Waters, water/1:1 acetonitrile-methanol as eluents [0.1% v/v formic acid buffered] 0% to 100%).

LRMS (m/z): 384 (M+1)⁺.

¹H-NMR δ (CDCl₃): 1.00 (s, 9H), 3.47 (d, 2H), 5.10 (t, 1H), 7.43 (ddd, 1H), 7.51 (dd, 2H), 7.81 (ddd, 2H), 8.15 (d, 1H), 8.66 (s, 1H), 8.78-8.71 (m, 2H), 10.63 (dd, 1H)

### EXAMPLE 14

### 3-{4-[(3-Fluorobenzyl)amino]pyrimidin-2-yl}imidazo[1,2-a]pyridine-6-carbonitrile

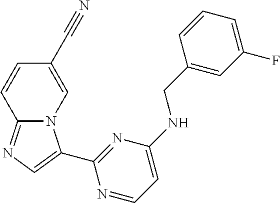 [SMILES: N#Cc1ccc2ncc(-c3nccc(NCc4cccc(F)c4)n3)n2c1]

Obtained as a white solid (17%) from 3-(4-hydroxypyrimidin-2-yl)imidazo[1,2-a]pyridine-6-carbonitrile (Preparation 4b) and (3-fluorophenyl)methanamine following the experimental procedure as described in Preparation 5a, followed by purification of the crude product by reverse phase chromatography (C-18 silica from Waters, water/1:1 acetonitrile-methanol as eluents [0.1% v/v formic acid buffered] 0% to 100%).

LRMS (m/z): 345 (M+1)⁺.

¹H-NMR δ (DMSO-d₆): 5.02 (bs, 2H), 6.64-6.97 (m, 1H), 7.29-7.46 (m, 1H), 7.59 (bs, 2H), 7.64-7.81 (m, 1H), 7.87-8.07 (m, 1H), 8.10-8.35 (m, 1H), 8.57 (m, 2H), 8.79 (bs, 1H), 10.66 (bs, 1H)

### EXAMPLE 15

### 3-{4-[(4-Fluorobenzyl)amino]pyrimidin-2-yl}imidazo[1,2-a]pyridine-6-carbonitrile

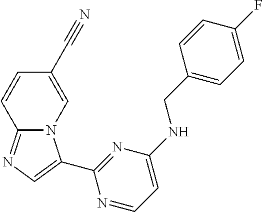 [SMILES: N#Cc1ccc2ncc(-c3nccc(NCc4ccc(F)cc4)n3)n2c1]

Obtained as a white solid (11%) from 3-(4-hydroxypyrimidin-2-yl)imidazo[1,2-a]pyridine-6-carbonitrile (Preparation 4b) and (4-fluorophenyl)methanamine following the experimental procedure as described in Preparation 5a, followed by purification of the crude product by reverse phase chromatography (C-18 silica from Waters, water/1:1 acetonitrile-methanol as eluents [0.1% v/v formic acid buffered] 0% to 100%).

LRMS (m/z): 345 (M+1)⁺.

¹H-NMR δ (CDCl₃): 4.65 (bs, 2H), 6.28 (d, 1H), 6.95-7.13 (m, 2H), 7.26 (s, 1H), 7.31-7.45 (m, 2H), 7.77 (d, 1H), 8.29 (d, 1H), 8.59 (s, 1H), 10.49 (bs, 1H)

### EXAMPLE 16

### 3-{4-[(2-Methylbenzyl)amino]pyrimidin-2-yl}imidazo[1,2-a]pyridine-6-carbonitrile

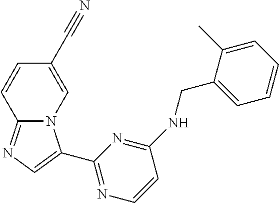 [SMILES: Cc1ccccc1CNc1ccnc(-c2cnc3ccc(C#N)cn23)n1]

Obtained as a white solid (39%) from 3-(4-hydroxypyrimidin-2-yl)imidazo[1,2-a]pyridine-6-carbonitrile (Preparation 4b) and o-tolylmethanamine following the experimental procedure as described in Preparation 5a followed by addition of water and extraction with ethyl acetate. The organic layer was washed with water, brine, dried (MgSO₄) and evaporated in vacuum and the residue was purified by flash chromatography (99:1 to 98:2 dichloromethane/methanol) to yield the desired compound.

LRMS (m/z): 341 (M+1)⁺.

¹H-NMR δ (DMSO-d₆): 2.43 (m, 3H), 4.52-4.85 (m, 2H), 6.38-6.73 (m, 1H), 7.10-7.30 (m, 3H), 7.30-7.44 (m, 1H), 7.60-7.82 (m, 1H), 7.91-7.94 (m, 1H), 8.04-8.20 (m, 1H), 8.20-8.36 (m, 1H), 8.43-8.61 (m, 1H), 10.40 (bs, 1H).

### EXAMPLE 17

### tert-Butyl-2-({[2-(6-cyanoimidazo[1,2-a]pyridin-3-yl)pyrimidin-4-yl]amino}methyl)piperidine-1-carboxylate

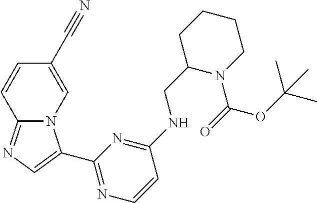 [SMILES: CC(C)(C)OC(=O)N1CCCCC1CNc1ccnc(-c2cnc3ccc(C#N)cn23)n1]

Obtained as a yellow solid (93%) from 3-(4-hydroxypyrimidin-2-yl)imidazo[1,2-a]pyridine-6-carbonitrile (Preparation 4b) and tert-butyl 2-(aminomethyl)piperidine-1-carboxylate (prepared as described in Chem Pharm Bull, 1998, 787-796) following the experimental procedure as described in Preparation 5a followed by addition of water and extraction with ethyl acetate. The organic layer was washed with water, brine, dried (MgSO₄) and evaporated in vacuum and the residue was purified by flash chromatography (98:2 dichloromethane/methanol) to give the title compound.

LRMS (m/z): 434 (M+1)⁺.

¹H-NMR δ (CDCl₃): 1.46 (s, 9H), 1.55-1.86 (m, 4H), 2.80-2.97 (m, 1H), 3.10-3.25 (m, 1H), 3.44-3.59 (m, 1H), 3.76-4.15 (m, 2H), 4.30-4.51 (m, 1H), 4.49-4.72 (m, 1H), 6.24 (bs, 1H), 7.42 (d, 1H), 7.78 (d, 1H), 8.22 (bs, 1H), 8.59 (s, 1H), 10.58 (bs, 1H)

### EXAMPLE 18

### 3-(4-{[(1-Acetylpiperidin-2-yl)methyl]amino}pyrimidin-2-yl)imidazo[1,2-a]pyridine-6-carbonitrile

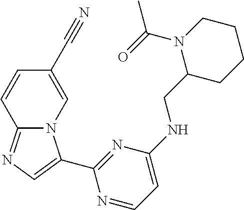 [SMILES: CC(=O)N1CCCCC1CNc1ccnc(-c2cnc3ccc(C#N)cn23)n1]

a) 3-{4-[(Piperidin-2-ylmethyl)amino]pyrimidin-2-yl}imidazo[1,2-a]pyridine-6-carbonitrile was obtained as a white solid (0.130 g, 80%) from tert-butyl 2-({[2-(6-cyanoimidazo[1,2-a]pyridin-3-yl)pyrimidin-4-yl]amino}methyl)piperidine-1-carboxylate (Example 17, 0.213 g, 0.49 mmol) following the experimental procedure as described in Preparation 5b. After the reaction was complete, water was added and the mixture was then neutralized with solid sodium hydrogen carbonate. The resultant precipitate was filtered, washed with water and dried in vacuum.

LRMS (m/z): 334 (M+1)⁺.

¹H-NMR δ (DMSO-d₆): 1.37-1.62 (m, 4H), 1.65-1.85 (m, 2H), 1.88-2.03 (m, 1H), 2.77-2.97 (m, 2H), 3.45-3.76 (m, 2H), 6.41-6.58 (m, 1H), 7.64-7.77 (m, 1H), 7.87 (bs, 1H), 7.90-7.98 (m, 1H), 8.18-8.40 (m, 1H), 8.58-8.79 (m, 1H), 10.46 (bs, 1H).

b) Triethylamine (0.13 mL, 0.95 mol) and acetyl chloride (0.05 mL, 0.73 mmol) were added sequentially to a stirred solution of 3-{4-[(piperidin-2-ylmethyl)amino]pyrimidin-2-yl}imidazo[1,2-a]pyridine-6-carbonitrile (0.100 g, 0.3 mmol) in dichloromethane (4 mL) and the mixture was stirred at ambient temperature for 16 hours. The mixture was evaporated in vacuum and then taken up in dichloromethane and the organic layer was washed with 4% aqueous sodium hydrogen carbonate solution, water, brine, dried (MgSO₄) and evaporated. Purification of the residue by flash chromatography (98:2 to 96:4 dichloromethane/methanol) gave the title compound (0.040 g, 35%) as a white solid.

LRMS (m/z): 376 (M+1)⁺.

¹H-NMR δ (DMSO-d₆): 1.11-1.88 (m, 6H), 2.56 (s, 3H), 3.46-3.95 (m, 2H) 4.14-4.31 (m, 1H), 4.29-4.51 (m, 1H), 4.95 (bs, 1H), 6.26-6.60 (m, 1H), 7.62-7.82 (m, 1H), 7.85-8.03 (m, 1H), 8.11-8.35 (m, 1H), 8.49-8.73 (m, 1H), 10.39-10.65 (m, 1H)

### EXAMPLE 19

### 3-[4-(Tetrahydro-2H-pyran-4-ylamino)pyrimidin-2-yl]imidazo[1,2-a]pyridine-6-carbonitrile

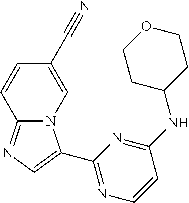 [SMILES: N#Cc1ccc2ncc(-c3nccc(NC4CCOCC4)n3)n2c1]

Obtained as a white solid (11%) from 3-(4-hydroxypyrimidin-2-yl)imidazo[1,2-a]pyridine-6-carbonitrile (Preparation 4b) and tetrahydro-2H-pyran-4-amine hydrochloride following the experimental procedure as described in Preparation 5a, followed by purification of the crude product by reverse phase chromatography (C-18 silica from Waters, water/1:1 acetonitrile-methanol as eluents [0.1% v/v formic acid buffered] 0% to 100%).

LRMS (m/z): 321 (M+1)⁺.

¹H-NMR δ (CDCl₃): 1.65 (d, 2H), 2.12 (d, 2H), 3.64 (d, 2H), 4.07 (d, 2H), 5.03 (bs, 1H), 6.26 (bs, 1H), 7.27 (d, 1H), 7.41 (dd, 1H), 7.79 (d, 1H), 8.27 (bs, 1H), 8.58 (bs, 1H), 10.54 (bs, 1H)

### EXAMPLE 20

### 3-(4-(8-Fluorochroman-4-ylamino)pyrimidin-2-yl)imidazo[1,2-a]pyridine-6-carbonitrile

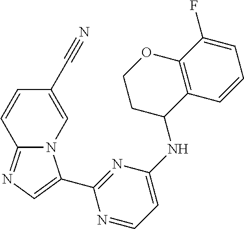 [SMILES: N#Cc1ccc2ncc(-c3nccc(NC4CCOc5c(F)cccc54)n3)n2c1]

Obtained as a white solid (8%) from 3-(4-hydroxypyrimidin-2-yl)imidazo[1,2-a]pyridine-6-carbonitrile (Preparation 4b) and (8-fluoro-3,4-dihydro-2H-chromen-4-yl)amine (prepared as described in WO2008043019) following the experimental procedure as described in Preparation 5a, followed by purification of the crude product by reverse phase chromatography (C-18 silica from Waters, water/1:1 acetonitrile-methanol as eluents [0.1% v/v formic acid buffered] 0% to 100%).

LRMS (m/z): 387 (M+1)⁺.

¹H-NMR δ (CDCl₃): 1.75 (bs, 1H), 2.46-2.26 (m, 2H), 4.51-4.30 (m, 2H), 6.32 (d, 1H), 6.87 (td, 1H), 7.11-7.02 (m, 2H), 7.42 (dd, 1H), 7.80 (d, 1H), 8.34 (d, 1H), 8.61 (s, 1H), 10.57 (s, 1H).

### EXAMPLE 21

### 3-[4-(Cyclohexylamino)pyrimidin-2-yl]imidazo[1,2-a]pyridine-6-carbonitrile

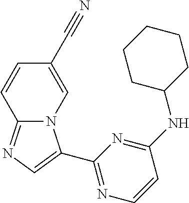 [SMILES: N#Cc1ccc2ncc(-c3nccc(NC4CCCCC4)n3)n2c1]

Obtained as an off-white solid (53%) from 3-(4-hydroxypyrimidin-2-yl)imidazo[1,2-a]pyridine-6-carbonitrile (Preparation 4b) and cyclohexanamine following the experimental procedure as described in Preparation 5a followed by addition of water and extraction with ethyl acetate. The organic layer was washed with water, brine, dried (MgSO₄) and evaporated. The residue was purified by flash chromatography (99:1 to 98:2 dichloromethane/methanol) to give the desired compound.

LRMS (m/z): 319 (M+1)⁺.

¹H-NMR δ (CDCl₃): 1.13-1.40 (m, 6H), 1.39-1.51 (m, 1H), 1.64-1.77 (m, 1H), 1.77-1.89 (m, 2H), 1.95-2.25 (m, 2H), 6.21 (d, 1H), 7.41 (d, 1H), 7.77 (d, 1H), 8.15-8.37 (m, 1H), 8.58 (s, 1H), 10.54 (s, 1H)

### EXAMPLE 22

### 3-{4-[(Trans-4-hydroxycyclohexyl)amino]pyrimidin-2-yl}imidazo[1,2-a]pyridine-6-carbonitrile

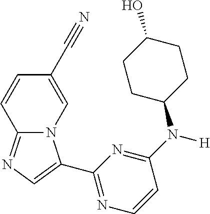 [SMILES: N#Cc1ccc2ncc(-c3nccc(N[C@H]4CC[C@H](O)CC4)n3)n2c1]

Obtained as a solid (17%) from 3-(4-hydroxypyrimidin-2-yl)imidazo[1,2-a]pyridine-6-carbonitrile (Preparation 4b) and trans-4-aminocyclohexanol following the experimental procedure as described in Preparation 5a, followed by purification of the crude product by reverse phase chromatography (C-18 silica from Waters, water/acetonitrile/methanol as eluents [0.1% v/v formic acid buffered] 0% to 100%).

LRMS (m/z): 335 (M+1)⁺.

¹H-NMR δ (CDCl₃): 1.61-1.32 (m, 4H), 2.17 (dd, 4H), 2.67-2.61 (m, 1H), 3.73 (ddd, 1H), 6.23 (d, 1H), 7.40 (dd, 1H), 7.79 (d, 1H), 8.27 (d, 1H), 8.58 (s, 1H), 10.54 (s, 1H).

### EXAMPLE 23

### 3-{4-[(5-Hydroxy-2-adamantyl)amino]pyrimidin-2-yl}imidazo[1,2-a]pyridine-6-carbonitrile

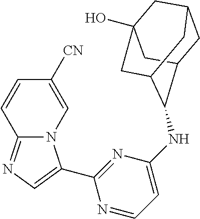

Obtained as a solid (1.6%) from 3-(4-hydroxypyrimidin-2-yl)imidazo[1,2-a]pyridine-6-carbonitrile (Preparation 4b) and (E)-4-aminotricyclo[3.3.1.1^{3,7}]decan-1-ol (Preparation 40) following the experimental procedure as described in Preparation 5a, followed by purification by reverse phase chromatography (C-18 silica from Waters, water/1:1 acetonitrile-methanol as eluents [0.1% v/v formic acid buffered] 0% to 100%).

LRMS (m/z): 387 (M+1)⁺.

### EXAMPLE 24

### 3-{4-[(5-Hydroxy-2-adamantyl)amino]pyrimidin-2-yl}imidazo[1,2-a]pyridine-6-carbonitrile

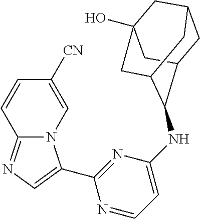

Obtained as a solid (6%) from 3-(4-hydroxypyrimidin-2-yl)imidazo[1,2-a]pyridine-6-carbonitrile (Preparation 4b) and (Z)-4-aminotricyclo[3.3.1.1^{3,7}]decan-1-ol (Preparation 41) following the experimental procedure as described in Preparation 5a, followed by purification by reverse phase chromatography (C-18 silica from Waters, water/1:1 acetonitrile-methanol as eluents [0.1% v/v formic acid buffered] 0% to 100%).

LRMS (m/z): 387 (M+1)⁺.

¹H-NMR δ (CDCl₃): 2.00-1.64 (m, 13H), 6.26 (d, 1H), 7.42 (dd, 1H), 7.82 (d, 1H), 8.27 (d, 1H), 8.58 (s, 1H), 10.51 (s, 1H).

### EXAMPLE 25

### 2-{4-[(2,2-Dimethylpropyl)amino]quinazolin-2-yl}imidazo[1,2-a]pyridine-6-carbonitrile

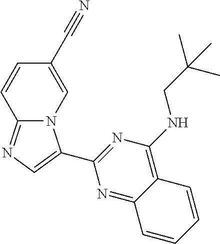 [SMILES: CC(C)(C)CNc1nc(-c2cnc3ccc(C#N)cn23)nc2ccccc12]

Obtained as a solid (14%) from 3-(4-hydroxyquinazolin-2-yl)imidazo[1,2-a]pyridine-6-carbonitrile (Preparation 24b) and 2,2-dimethylpropan-1-amine following the experimental procedure as described in Preparation 5a, followed by purification of the crude product by reverse phase chromatography (C-18 silica from Waters, water/1:1 acetonitrile-methanol as eluents [0.1% v/v formic acid buffered] 0% to 100%).

LRMS (m/z): 357 (M+1)⁺.

¹H-NMR δ (CDCl₃): 1.11 (s, 9H), 3.67 (d, 2H), 5.87 (t, 2H), 7.42 (dd, 1H), 7.49 (ddd, 1H), 7.75-7.70 (m, 1H), 7.83-7.76 (m, 2H), 7.97-7.91 (m, 1H), 8.70 (s, 1H), 10.88 (dd, 1H)

### EXAMPLE 26

### 3-{4-[Benzyl(methyl)amino]pyrimidin-2-yl}imidazo[1,2-a]pyridine-6-carbonitrile

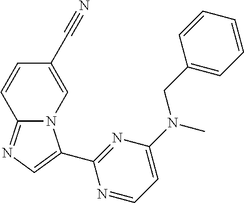 [SMILES: CN(Cc1ccccc1)c1ccnc(-c2cnc3ccc(C#N)cn23)n1]

An oven dried resealable Schlenk tube was charged with imidazo[1,2-a]pyridine-6-carbonitrile (Preparation 1, 0.30 g, 2.1 mmol), N-benzyl-2-chloro-N-methylpyrimidin-4-amine (Preparation 31, 0.24 g, 1.0 mmol), potassium acetate (0.21 g, 2.1 mmol) and N,N′-dimethylacetamide (3 mL). The Schlenk tube was subjected to three cycles of evacuation-backfilling with argon then tetrakis(triphenylphosphine)palladium (0) (0.12 g, 0.10 mmol) was added. After three further cycles of evacuation-backfilling with argon, the Schlenk tube was capped and placed in an oil bath at 150° C. and the mixture was stirred overnight. The mixture was evaporated in vacuum and the residue was partitioned between ethyl acetate and water. The organic layer was washed with brine, dried (MgSO₄) and evaporated and the residue was purified by flash chromatography (99:1 to 98:2 dichloromethane/methanol) to give the title compound (0.105 g, 30%) as a pale brown solid.

LRMS (m/z): 341 (M+1)+.

¹H-NMR δ (CDCl₃): 3.22 (bs, 3H), 4.91 (bs, 2H), 6.40 (d, 1H), 7.27-7.41 (m, 6H), 7.76 (dd, 1H), 8.31 (d, 1H), 8.59 (s, 1H), 10.49 (bs, 1H)

### EXAMPLE 27

### 3-(4-{[(1S)-1-Phenylethyl]amino}pyrimidin-2-yl)imidazo[1,2-a]pyridine-6-carboxylic acid

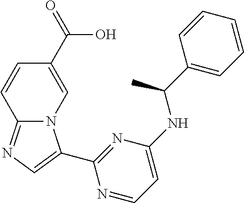 [SMILES: C[C@H](Nc1ccnc(-c2cnc3ccc(C(=O)O)cn23)n1)c1ccccc1]

A mixture of 3-[4-((S)-1-Phenyl-ethylamino)-pyrimidin-2-yl]-imidazo[1,2-a]pyridine-6-carbonitrile (Example 1, 0.037 g, 0.11 mmol) and potassium hydroxide (0.033 g, 0.59 mmol) in ethylene glycol (0.5 mL) was heated to 150° C. and stirred overnight. The mixture was then cooled to room temperature, water (5 mL) was added and the mixture was extracted with ethyl acetate. The aqueous layer was acidified to a pH of 5 with 5M aqueous hydrochloric acid and the suspension was extracted with chloroform. The organic layer was dried (MgSO₄) and evaporated to give the title compound (0.010 g, 25%) as an off-white solid.

LRMS (m/z): 360 (M+1)⁺, 358 (M−1)⁻.

¹H-NMR δ (DMSO-d₆): 1.51 (d, 3H), 5.43 (bs, 1H), 6.46 (d, 1H), 7.20 (d, 1H), 7.31 (t, 2H), 7.50 (d, 2H), 7.73 (d, 1H), 7.83 (d, 1H), 8.13 (d, 1H), 8.18 (d, 1H), 8.36 (s, 1H), 10.63 (bs, 1H)

### EXAMPLE 28

### 2-(6-Fluoroimidazo[1,2-a]pyridin-3-yl)-N-[(1S)-1-phenylethyl]pyrimidin-4-amine

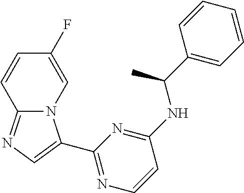 [SMILES: C[C@H](Nc1ccnc(-c2cnc3ccc(F)cn23)n1)c1ccccc1]

a) 2-(6-Fluoroimidazo[1,2-a]pyridin-3-yl)-N-(4-methoxybenzyl)-N-[(1S)-1-phenylethyl]pyrimidin-4-amine was obtained as a colorless oil (0.029 g, 8%) from 6-fluoroimidazo[1,2-a]pyridine (Preparation 2, 0.127 g, 0.93 mmol), 2-chloro-N-(4-methoxybenzyl)-N-[(1S)-1-phenylethyl]pyrimidin-4-amine (Preparation 30b, 0.300 g, 0.85 mmol), potassium acetate (0.138 g, 1.41 mmol) and tetrakis(triphenylphosphine)palladium (0) (0.098 g, 0.08 mmol) following the experimental procedure as described in Preparation 29. The crude product was purified by flash chromatography (1:1 hexanes/ethyl acetate).

LRMS (m/z): 454 (M+1)⁺

¹H-NMR δ (CDCl₃): 1.68 (d, 3H), 3.78 (s, 3H), 4.41-4.69 (m, 3H), 6.20 (d, 1H), 6.84 (d, 2H), 7.11 (d, 2H), 7.14-7.24 (m, 1H), 7.26-7.39 (m, 5H), 7.64-7.69 (m, 1H), 8.22 (d, 1H), 8.52 (s, 1H), 9.81 (bs, 1H).

b) A mixture of 2-(6-fluoroimidazo[1,2-a]pyridin-3-yl)-N-(4-methoxybenzyl)-N-[(1S)-1-phenylethyl]pyrimidin-4-amine (0.029 g, 0.06 mmol) and trifluoroacetic acid (4 mL) was stirred and heated to 65° C. in a sealed tube. After 19 hours, the mixture was evaporated in vacuum and 2M aqueous hydrochloric acid and ethyl acetate were added to the residue. The aqueous layer was basified with solid sodium hydrogen carbonate and then extracted with chloroform. The organic layer was dried (MgSO₄), evaporated and the residue was purified by flash chromatography (100:1 to 50:1 dichloromethane/methanol), followed by purification by reverse phase chromatography (C-18 silica from Waters, water/1:1 acetonitrile-methanol as eluents [0.1% v/v formic acid buffered] 0% to 100%) to give the title compound (0.009 g, 42%) as a white solid.

LRMS (m/z): 334 (M+1)⁺.

¹H-NMR δ (DMSO-d₆): 1.56 (d, 3H), 5.29 (bs, 1H), 6.52 (bs, 1H), 7.17-7.31 (m, 1H), 7.30-7.43 (m, 2H), 7.49 (d, 2H), 7.52-7.61 (m, 1H), 7.73-7.88 (m, 1H), 8.21 (d, 2H), 8.37 (bs, 1H), 9.78 (bs, 1H).

### EXAMPLE 29

### Trans-4-{[2-(6-fluoroimidazo[1,2-a]pyridin-3-yl)pyrimidin-4-yl]amino}cyclohexanol

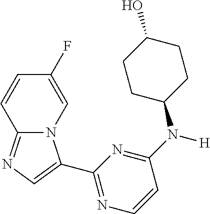 [SMILES: O[C@H]1CC[C@H](Nc2ccnc(-c3cnc4ccc(F)cn34)n2)CC1]

Obtained as a yellow solid (70%) from 2-(6-fluoroimidazo[1,2-a]pyridin-3-yl)pyrimidin-4-ol (Preparation 8b) and trans-4-aminocyclohexanol following the experimental procedure as described in Preparation 5a, followed by purification of the crude product by flash chromatography (dichloromethane/methanol 100:10).

LRMS (m/z): 328 (M+1)⁺.

¹H-NMR δ (CDCl₃): 1.38 (dd, 2H), 1.51 (dd, 2H), 2.08 (d, 2H), 2.20 (d, 2H), 3.67 (s, 1H), 5.74 (d, 1H), 6.22 (d, 1H), 7.29-7.16 (m, 1H), 7.73-7.59 (m, 1H), 8.19 (s, 1H), 8.50 (s, 1H), 10.02 (s, 1H).

### EXAMPLE 30

### 3-(4-{[(2S,7S)-5-Hydroxy-2-adamantyl]amino}pyrimidin-2-yl)imidazo[1,2-a]pyridine-6-carbonitrile

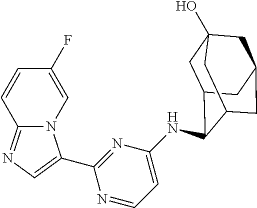

Obtained as a solid (30%) from 2-(6-fluoroimidazo[1,2-a]pyridin-3-yl)pyrimidin-4-ol (Preparation 8b) and (4S,7S)-4-aminotricyclo[3.3.1.1^{3,7}]decan-1-ol following the same experimental procedure as described in Preparation 5a with the exception that the reaction was carried out at 60° C. The crude product was purified by flash chromatography (9:1 dichloromethane/methanol).

LRMS (m/z): 380 (M+1)⁺.

¹H-NMR δ (DMSO-d₆): 1.09 (bs, 1H), 1.40-2.02 (m, 10H), 1.63 (bs, 1H), 2.10 (bs, 1H), 2.29 (bs, 1H), 4.07 (bs, 1H), 4.46 (s, 1H), 6.56 (d, 1H), 7.53 (d, 1H), 7.81 (dd, 1H), 8.13 (d, 1H), 8.37 (s, 1H), 9.96 (bs, 1H)

### EXAMPLE 31

### N-(2,2-Dimethylpropyl)-2-imidazo[1,2-a]pyridin-3-ylpyrimidin-4-amine

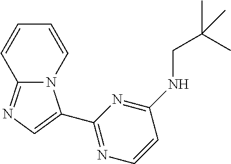 [SMILES: CC(C)(C)CNc1ccnc(-c2cnc3ccccn23)n1]

Obtained as a off-white solid (45%) from 2-imidazo[1,2-a]pyridin-3-ylpyrimidin-4-ol (Preparation 14b) and neopentylamine following the experimental procedure as described in Preparation 5a followed by addition of water and extraction with ethyl acetate. The organic layer was washed with water, brine, dried (MgSO₄) and evaporated. The residue was treated with isopropyl alcohol and the solid was collected by filtration and dried in vacuum to give the title compound.

LRMS (m/z): 282 (M+1)⁺.

¹H-NMR δ (DMSO-d₆): 1.01 (s, 9H), 6.48 (d, 1H), 7.18 (t, 1H), 7.49-7.39 (m, 1H), 7.55 (t, 1H), 7.77 (d, 1H), 8.17 (s, 1H), 8.39 (s, 1H), 10.08-9.92 (m, 1H).

### EXAMPLE 32

### N-(2,2-Dimethylpropyl)-2-imidazo[1,2-a]pyridin-3-yl-6-pyridin-3-ylpyrimidin-4-amine

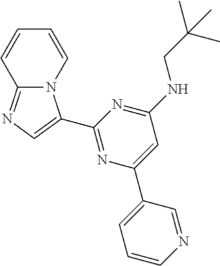 [SMILES: CC(C)(C)CNc1cc(-c2cccnc2)nc(-c2cnc3ccccn23)n1]

Diisopropylethylamine (0.07 mL, 0.40 mmol) and neopentylamine (0.05 mL, 0.42 mmol) were added to a stirred solution of 3-(4-chloro-6-pyridin-3-ylpyrimidin-2-yl)imidazo[1,2-a]pyridine (Preparation 27d, 0.037 g, 0.11 mmol) in N,N′-dimethylformamide (1.0 mL) and the mixture was heated to 90° C. under an argon atmosphere. After 16 hours the mixture was evaporated in vacuum and then taken up in dichloromethane. The organic layer was washed with water and brine, dried (MgSO₄) and evaporated to give the title compound (0.027 g, 60%).

LRMS (m/z): 359 (M+1)⁺.

¹H-NMR δ (CDCl₃): 1.07 (s, 9H), 3.29 (s, 2H), 5.25 (s, 1H), 6.61 (s, 1H), 7.00 (t, 1H), 7.38-7.29 (m, 1H), 7.47 (dd, 1H), 7.74 (d, 1H), 8.40-8.33 (m, 1H), 8.61 (s, 1H), 8.73 (dd, 1H), 9.28 (s, 1H), 10.04 (d, 1H).

### EXAMPLE 33

### 3-(6-{[(1S)-1-Phenylethyl]amino}pyrazin-2-yl)imidazo[1,2-a]pyridine-6-carbonitrile

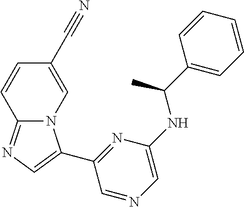 [SMILES: C[C@H](Nc1cncc(-c2cnc3ccc(C#N)cn23)n1)c1ccccc1]

Obtained as a green solid (73%) from 6-chloro-N-(1-phenylethyl)pyrazin-2-amine (Preparation 32) and imidazo[1,2-a]pyridine-6-carbonitrile (Preparation 1) following the same experimental procedure as described in Example 26 followed by purification of the crude product by flash chromatography (99:1 to 98:2 dichloromethane/methanol).

LRMS (m/z): 341 (M+1)⁺.

¹H-NMR δ (CDCl₃): 1.69 (d, 3H), 5.04 (t, 1H), 5.21 (d, 1H), 7.27-7.32 (m, 1H), 7.34-7.51 (m, 5H), 7.75 (d, 1H), 7.81 (s, 1H), 8.25 (s, 1H), 8.33 (s, 1H), 10.06 (s, 1H)

### EXAMPLE 34

### 3-{6-[(Cyclohexylmethyl)amino]pyrazin-2-yl}imidazo[1,2-a]pyridine-6-carbonitrile

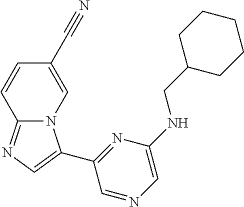 [SMILES: N#Cc1ccc2ncc(-c3cncc(NCC4CCCCC4)n3)n2c1]

Obtained as a solid (26%) from 3-(6-hydroxypyrazin-2-yl)imidazo[1,2-a]pyridine-6-carbonitrile (Preparation 29, 0.052 g, 0.22 mmol) and cyclohexylmethanamine (0.11 mL, 0.88 mmol) following the experimental procedure as described in Preparation 5a.

LRMS (m/z): 333 (M+1)⁺.

¹H-NMR δ (DMSO-d₆): 0.97-1.13 (m, 2H), 1.25 (bs, 4H), 1.74 (bs, 4H), 1.91 (d, 1H), 3.27 (t, 2H), 7.57 (t, 1H), 7.69 (d, 1H), 7.88-7.96 (m, 1H), 8.40 (s, 1H), 8.61 (s, 1H), 10.41 (s, 1H)

### EXAMPLE 35

### 6-(6-Fluoroimidazo[1,2-a]pyridin-3-yl)-N-[(1S)-1-phenylethyl]pyrazin-2-amine

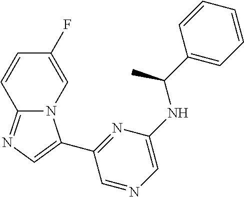 [SMILES: C[C@H](Nc1cncc(-c2cnc3ccc(F)cn23)n1)c1ccccc1]

Obtained as a green solid (25%) from (S)-6-chloro-N-(1-phenylethyl)pyrazin-2-amine (Preparation 32) and 6-fluoroimidazo[1,2-a]pyridine (Preparation 2) following the experimental procedure as described in Example 26 followed by purification of the crude product by reverse phase chromatography (C-18 silica from Waters, water/1:1 acetonitrile-methanol as eluents [0.1% v/v formic acid buffered] 0% to 100%).

LRMS (m/z): 334 (M+1)⁺.

¹H-NMR δ (CDCl₃): 1.66 (d, 3H), 5.01-5.12 (m, 2H), 7.15-7.23 (m, 1H), 7.33-7.46 (m, 5H), 7.63 (dd, 1H), 7.76 (s, 1H), 8.15 (s, 1H), 8.28 (s, 1H), 9.39 (dd, 1H)

### EXAMPLE 36

### 6-(6-Chloroimidazo[1,2-a]pyridin-3-O—N-[(1S)-1-phenylethyl]pyrazin-2-amine

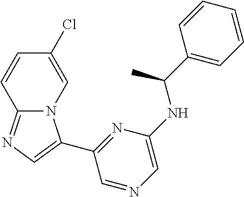 [SMILES: C[C@H](Nc1cncc(-c2cnc3ccc(Cl)cn23)n1)c1ccccc1]

Obtained as a green solid (71%) from (S)-6-chloro-N-(1-phenylethyl)pyrazin-2-amine (Preparation 32) and 6-chloroimidazo[1,2-a]pyridine (Preparation 3) following the experimental procedure as described in Example 26 followed by purification of the crude product by flash chromatography (99:1 to 97:3 dichloromethane/methanol).

LRMS (m/z): 350 (M+1)⁺.

¹H-NMR δ (CDCl₃): 1.60 (s, 3H), 5.08 (bs, 2H), 7.29 (bs, 2H), 7.37 (t, 2H), 7.41-7.50 (m, 1H), 7.64 (d, 1H), 7.75 (s, 1H), 8.16 (s, 1H), 8.30 (s, 1H), 9.72 (s, 1H)

### EXAMPLE 37

### (S)-6-(Imidazo[1,2-a]pyridin-3-yl)-N-(1-phenylethyl)pyrazin-2-amine

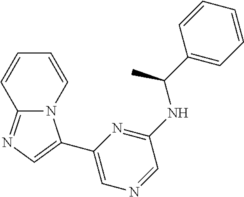 [SMILES: C[C@H](Nc1cncc(-c2cnc3ccccn23)n1)c1ccccc1]

To a solution of 6-(6-chloroimidazo[1,2-a]pyridin-3-yl)-N-[(1S)-1-phenylethyl]pyrazin-2-amine (Example 36, 0.100 g, 0.29 mmol) in ethanol (2 mL) was added 10% palladium on charcoal (0.006 g) and ammonium formate (0.180 g, 2.9 mmol). The mixture was stirred and heated to reflux for 24 hours. Further palladium (0.006 g) and ammonium formate (0.180 g) were added and stirring at reflux was continued for 24 hours. This process was repeated until little starting material remained. The reaction mixture was then filtered, evaporated and the residue was purified by reverse phase chromatography (C-18 silica from Waters, water/1:1 acetonitrile-methanol as eluents [0.1% v/v formic acid buffered] 0% to 100%) to give the title compound (0.023 g, 25%) as a pale green solid.

LRMS (m/z): 316 (M+1)⁺.

¹H-NMR δ (CDCl₃): 1.64 (d, 3H), 5.00 (bs, 2H), 6.66 (t, 1H), 7.28-7.34 (m, 0H), 7.40 (bs, 4H), 7.65 (d, 1H), 7.78 (s, 1H), 8.10 (s, 1H), 8.27 (s, 1H), 8.95 (d, 1H)

### EXAMPLE 38

### 6-(6-Cyclopropylimidazo[1,2-a]pyridin-3-yl)-N-[(1S)-1-phenylethyl]pyrazin-2-amine

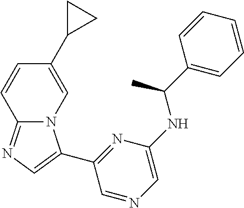 [SMILES: C[C@H](Nc1cncc(-c2cnc3ccc(C4CC4)cn23)n1)c1ccccc1]

Nitrogen was bubbled for 5 minutes through a mixture of (S)-6-(6-chloroimidazo[1,2-a]pyridin-3-yl)-N-(1-phenylethyl)pyrazin-2-amine (Example 36, 0.085 g, 0.24 mmol), cyclopropylboronic acid (0.081 g, 0.94 mmol) and potassium phosphate (0.180 g, 0.85 mmol) in toluene (1.0 mL) and water (0.1 mL) contained in a microwave vessel. Then palladium (II) acetate (0.004 g, 0.01 mmol) and tricyclohexylphosphine (0.014 mg, 0.05 mmol) were added and the vessel was sealed and subjected to microwave irradiation for 7 hours at 150° C. The reaction was partitioned between dichloromethane and water and the organic phase was dried (MgSO₄) and the residue was purified by reverse phase chromatography (C-18 silica from Waters, water/1:1 acetonitrile-methanol as eluents [0.1% v/v formic acid buffered] 0% to 100%) to give the title compound (0.025 g, 29%) as a pale green solid.

LRMS (m/z): 356 (M+1)⁺.

¹H-NMR δ (CDCl₃): 0.73 (bs, 2H), 0.99 (d, 2H), 1.69 (d, 3H), 1.87 (bs, 1H), 4.95 (d, 1H), 5.05-5.25 (m, 1H), 7.00 (d, 1H), 7.28-7.48 (m, 5H), 7.59 (d, 1H), 7.69 (d, 1H), 8.10 (d, 1H), 8.29 (d, 1H), 9.42 (s, 1H)

### EXAMPLE 39

### N-[(1S)-1-Phenylethyl]-6-(6-pyridin-3-ylimidazo[1,2-a]pyridin-3-yl)pyrazin-2-amine

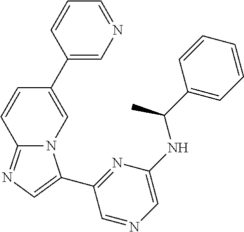 [SMILES: C[C@H](Nc1cncc(-c2cnc3ccc(-c4cccnc4)cn23)n1)c1ccccc1]

Nitrogen was bubbled for 5 minutes through a mixture of (S)-6-(6-chloroimidazo[1,2-a]pyridin-3-yl)-N-(1-phenylethyl)pyrazin-2-amine (Example 36, 0.080 g, 0.23 mmol), pyridin-3-yl boronic acid (0.056 g, 0.46 mmol) and potassium carbonate (0.063 g, 0.46 mmol) in 1,4-dioxane (1.0 mL) and ethanol (0.5 mL) contained in a microwave vessel. Then tetrakis(triphenylphosphine)palladium (0) (0.026 g, 0.02 mmol) was added and the vessel was sealed and subjected to microwave irradiation for 3 hours at 150° C. The solvent was removed under reduced pressure and the residue was partitioned between dichloromethane and water. The organic layer was dried (MgSO₄) and evaporated and the residue was purified by flash chromatography (98:2 to 96:4 dichloromethane/methanol) to give the title compound (0.040 g, 45%) as a pale green solid.

LRMS (m/z): 393 (M+1)⁺.

¹H-NMR δ (DMSO-d₆): 1.48 (d, 3H), 5.14 (quintet, 1H), 7.08-7.18 (m, 4H), 7.58 (ddd, 1H), 7.70-7.86 (m, 4H), 7.90 (s, 1H), 8.16 (ddd, 1H), 8.41 (d, 2H), 8.70 (dd, 1H), 8.99 (dd, 1H), 10.01 (dd, 1H)

### EXAMPLE 40

### N-[(1S)-1-Phenylethyl]-6-(6-pyridin-4-ylimidazo[1,2-a]pyridin-3-yl)pyrazin-2-amine

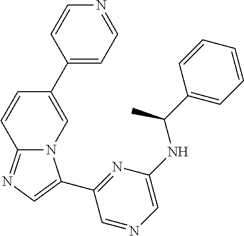 [SMILES: C[C@H](Nc1cncc(-c2cnc3ccc(-c4ccncc4)cn23)n1)c1ccccc1]

Obtained as a solid (36%) from (S)-6-(6-chloroimidazo[1,2-a]pyridin-3-yl)-N-(1-phenylethyl)pyrazin-2-amine (Example 36) and 4-(4,4,5,5-tetramethyl-1,3,2-dioxaborolan-2-yl)pyridine following the experimental procedure as described in Example 39 followed by purification of the crude product by flash chromatography (98:2 to 95:5 dichloromethane/methanol).

LRMS (m/z): 393 (M+1)⁺.

¹H-NMR δ (CDCl₃): 1.65 (d, 3H), 5.00 (d, 1H), 5.16 (t, 1H), 7.46-7.58 (m, 3H), 7.74 (s, 1H), 7.82 (d, 1H), 8.21 (s, 1H), 8.35 (s, 1H), 8.69 (d, 2H), 10.00 (s, 1H)

### EXAMPLE 41

### N-[(1S)-1-Phenylethyl]-6-[6-(1H-pyrazol-4-yl)imidazo[1,2-a]pyridin-3-yl]pyrazin-2-amine

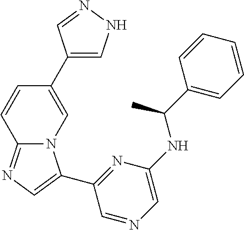 [SMILES: C[C@H](Nc1cncc(-c2cnc3ccc(-c4cn[nH]c4)cn23)n1)c1ccccc1]

Obtained as a pale green solid (23%) from (S)-6-(6-chloroimidazo[1,2-a]pyridin-3-yl)-N-(1-phenylethyl)pyrazin-2-amine (Example 36) and 4-(4,4,5,5-tetramethyl-1,3,2-dioxaborolan-2-yl)-1H-pyrazole following the experimental procedure as described in Example 39 followed by purification of the crude product by flash chromatography (98:2 to 96:4 dichloromethane/methanol).

LRMS (m/z): 382 (M+1)⁺.

¹H-NMR δ (DMSO-d₆): 1.61 (bs, 3H), 5.26 (bs, 1H), 7.14-7.46 (m, 5H), 7.64-8.48 (m, 7H), 9.94 (bs, 1H)

### EXAMPLE 42

### N-[(1S)-1-Phenylethyl]-6-(6-phenylimidazo[1,2-a]pyridin-3-yl)pyrazin-2-amine

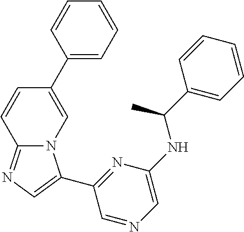 [SMILES: C[C@H](Nc1cncc(-c2cnc3ccc(-c4ccccc4)cn23)n1)c1ccccc1]

Obtained as a pale green solid (38%) from (S)-6-(6-chloroimidazo[1,2-a]pyridin-3-yl)-N-(1-phenylethyl)pyrazin-2-amine (Example 36) and phenylboronic acid following the experimental procedure as described in Example 39 followed by purification of the crude product by flash chromatography (98:2 to 97:3 dichloromethane/methanol).

LRMS (m/z): 392 (M+1)⁺.

¹H-NMR δ (CDCl₃): 1.25 (bs, 3H), 4.96 (bs, 1H), 5.15 (bs, 1H), 7.35 (bs, 7H), 7.40-7.86 (m, 5H), 8.19 (bs, 1H), 8.34 (bs, 1H), 8.76 (bs, 1H), 9.93 (bs, 1H)

### EXAMPLE 43

### 6-[6-(4-Fluorophenyl)imidazo[1,2-a]pyridin-3-yl]-N-[(1S)-1-phenylethyl]pyrazin-2-amine

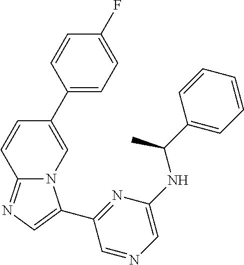 [SMILES: C[C@H](Nc1cncc(-c2cnc3ccc(-c4ccc(F)cc4)cn23)n1)c1ccccc1]

Obtained as a pale green solid (32%) from (S)-6-(6-chloroimidazo[1,2-a]pyridin-3-yl)-N-(1-phenylethyl)pyrazin-2-amine (Example 36) and 4-fluorophenylboronic acid following the experimental procedure as described in Example 39 followed by purification of the crude product by flash chromatography (98:2 to 97:3 dichloromethane/methanol).

LRMS (m/z): 410 (M+1)⁺.

¹H-NMR δ (CDCl₃): 1.58 (s, 3H), 2.18 (s, 1H), 7.17 (t, 1H), 7.22-7.30 (m, 9H), 7.46-7.60 (m, 1H), 7.76 (dd, 1H), 8.19 (s, 1H), 8.33 (s, 1H), 9.86 (dd, 1H)

### EXAMPLE 44

### 3-(4-{[(3R)-1-(Ethylsulfonyl)piperidin-3-yl]amino}pyrimidin-2-yl)imidazo[1,2-a]pyridine-6-carbonitrile

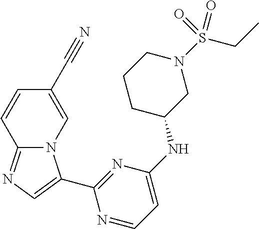 [SMILES: CCS(=O)(=O)N1CCC[C@@H](Nc2ccnc(-c3cnc4ccc(C#N)cn34)n2)C1]

Triethylamine (0.064 mL, 0.46 mmol) and ethanesulfonyl chloride (0.032 mL, 0.34 mmol) were added sequentially to a cooled (ice-bath), stirred solution of 3-{4-[(3R)-piperidin-3-ylamino]pyrimidin-2-yl}imidazo[1,2-a]pyridine-6-carbonitrile (Preparation 5b, 0.072 g, 0.23 mmol) in dichloromethane (3 mL). The mixture was warmed to ambient temperature and stirred for 3 hours. Water was then added and the organic layer was washed with water, brine, dried (MgSO₄) and evaporated. The residue was purified by flash chromatography (98:2 to 96:4 dichloromethane/methanol) to give the title compound (0.052 g, 56%) as a white solid.

LRMS (m/z): 412 (M+1)⁺.

¹H-NMR δ (CDCl₃): 1.47-1.27 (m, 4H), 1.94 (s, 4H), 3.08-2.87 (m, 3H), 3.49 (s, 3H), 5.33 (s, 1H), 6.32 (s, 1H), 7.77 (d, 1H), 8.27 (s, 1H), 8.60 (s, 1H), 10.55 (t, 1H).

### EXAMPLE 45

### 3-(4-{[(3R)-1-(Isopropylsulfonyl)piperidin-3-yl]amino}pyrimidin-2-yl)imidazo[1,2-a]pyridine-6-carbonitrile

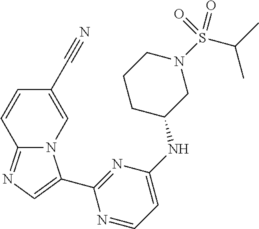 [SMILES: CC(C)S(=O)(=O)N1CCC[C@@H](Nc2ccnc(-c3cnc4ccc(C#N)cn34)n2)C1]

Obtained as a solid (18%) from 3-{4-[(3R)-piperidin-3-ylamino]pyrimidin-2-yl}imidazo[1,2-a]pyridine-6-carbonitrile (Preparation 5b) and propane-2-sulfonyl chloride following the same experimental procedure described in Example 44. The crude product was purified by reverse phase chromatography (C-18 silica from Waters, water/1:1 acetonitrile-methanol as eluents [0.1% v/v formic acid buffered] 0% to 100%).

LRMS (m/z): 426 (M+1)⁺.

¹H-NMR δ (CDCl₃): 1.37 (dd, 6H), 1.67-2.09 (m, 4H), 2.88-2.98 (m, 1H), 3.22 (dt, 1H), 3.70 (bs, 4H), 5.41 (bs, 1H), 6.35 (bs, 1H), 7.40 (d, 1H), 7.79 (d, 1H), 8.28 (bs, 1H), 8.61 (s, 1H), 10.55 (s, 1H)

### EXAMPLE 46

### 3-(4-{[(3R)-1-(Cyanoacetyl)piperidin-3-yl]amino}pyrimidin-2-yl)imidazo[1,2-a]pyridine-6-carbonitrile

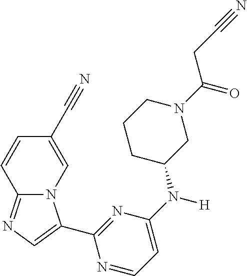 [SMILES: N#CCC(=O)N1CCC[C@@H](Nc2ccnc(-c3cnc4ccc(C#N)cn34)n2)C1]

Triethylamine (0.10 mL, 0.72 mmol) and 3-[(2,5-dioxopyrrolidin-1-yl)oxy]-3-oxopropanenitrile (prepared as described in BE875054(A1), 0.148 g, 0.81 mmol) were added sequentially to a stirred solution of 3-{4-[(3R)-piperidin-3-ylamino]pyrimidin-2-yl}imidazo[1,2-a]pyridine-6-carbonitrile (Preparation 5b, 0.200 g, 0.63 mmol) in dichloromethane (5 mL). The mixture was stirred at ambient temperature for 24 hours and then was evaporated. Water was added and the mixture was extracted with dichloromethane. The organic layer was dried (MgSO₄), evaporated and the residue was purified by flash chromatography (98:2 to 95:5 dichloromethane/methanol) to give the title compound (0.200 g, 83%) as an off-white solid.

LRMS (m/z): 387 (M+1)⁺.

¹H-NMR δ (DMSO-d₆): 1.67-1.49 (m, 2H), 1.89-1.70 (m, 2H), 2.11-1.98 (m, 2H), 2.51 (s, 2H), 3.66 (t, 1H), 3.90 (t, 1H), 4.66 (d, 1H), 6.44 (d, 1H), 7.69 (dt, 1H), 7.90 (ddd, 1H), 8.23 (s, 1H), 8.61 (s, 1H).

### EXAMPLE 47

### 3-(4-{[(3R)-1-Propionylpiperidin-3-yl]amino}pyrimidin-2-yl)imidazo[1,2-a]pyridine-6-carbonitrile

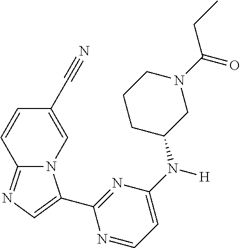 [SMILES: CCC(=O)N1CCC[C@@H](Nc2ccnc(-c3cnc4ccc(C#N)cn34)n2)C1]

Triethylamine (0.024 mL, 0.17 mmol) and propionyl chloride (0.036 mL, 0.42 mmol) were added sequentially to a stirred solution of 3-{4-[(3R)-piperidin-3-ylamino]pyrimidin-2-yl}imidazo[1,2-a]pyridine-6-carbonitrile (Preparation 5b, 0.050 g, 0.16 mmol) in dichloromethane (2 mL). The mixture was stirred at ambient temperature for 16 hours then evaporated in vacuum and the residue was taken up in dichloromethane. The organic layer was washed with water and brine, dried (MgSO₄) and evaporated. The residue was taken up in 2M aqueous hydrochloric acid and washed with ethyl acetate. The aqueous layer was neutralized with 2M aqueous sodium hydroxide solution and extracted with dichloromethane. The organic layer was dried (MgSO₄) and evaporated to give the title compound (0.032 g, 54%) as a white solid.

LRMS (m/z): 376 (M+1)⁺.

¹H-NMR δ (DMSO-d₆): 4.74 (d, 1H), 6.47 (d, 1H), 7.72 (s, 2H), 7.91 (d, 1H), 8.24 (s, 1H), 8.57 (d, 1H), 10.47 (s, 1H).

### EXAMPLE 48

### 3-[4-({(3R)-1-[(1-Cyanocyclopropyl)carbonyl]piperidin-3-yl}amino)pyrimidin-2-yl]imidazo[1,2-a]pyridine-6-carbonitrile

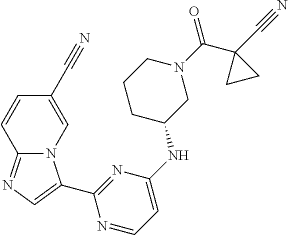 [SMILES: N#Cc1ccc2ncc(-c3nccc(N[C@@H]4CCCN(C(=O)C5(C#N)CC5)C4)n3)n2c1]

1,1′-Carbonyldiimidazole (0.040 g, 0.25 mmol) was added to a stirred solution of 1-cyanocyclopropanecarboxylic acid (0.041 g, 0.37 mmol) in dichloroethane (1 mL). After stirring 15 minutes at ambient temperature, 3-{4-[(3R)-piperidin-3-ylamino]pyrimidin-2-yl}imidazo[1,2-a]pyridine-6-carbonitrile (Preparation 5b, 0.040 g, 0.13 mmol) was added and the mixture was stirred overnight. The mixture was evaporated and the residue was purified by flash chromatography (98:2 to 95:5 dichloromethane/methanol) to give the title compound (0.035 g, 68%) as a white solid.

LRMS (m/z): 413 (M+1)⁺.

¹H-NMR δ (CDCl₃): 2.04-1.44 (m, 8H), 2.20 (s, 1H), 3.73-3.50 (m, 1H), 4.44-3.88 (m, 2H), 5.08 (bs, 1H), 6.35 (bs, 1H), 7.42 (dd, 1H), 7.79 (dd, 1H), 8.32 (d, 1H), 8.64 (s, 1H), 10.54 (s, 1H).

### EXAMPLE 49

### 3-(4-{[(3R)-1-(Methoxyacetyl)piperidin-3-yl]amino}pyrimidin-2-yl)imidazo[1,2-a]pyridine-6-carbonitrile

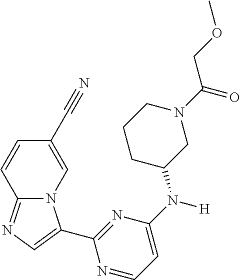

Obtained as an off-white solid (57%) from 3-{4-[(3R)-piperidin-3-ylamino]pyrimidin-2-yl}imidazo[1,2-a]pyridine-6-carbonitrile (Preparation 5b) and 2-methoxyacetic acid following the experimental procedure as described in Example 48. The crude product was purified by reverse phase chromatography (C-18 silica from Waters, water/1:1 acetonitrile-methanol as eluents [0.1% v/v formic acid buffered] 0% to 100%).

LRMS (m/z): 392 (M+1)⁺.

¹H-NMR δ (CDCl₃): 1.57 (s, 3H), 2.19-1.60 (m, 4H), 2.92 (d, 1H), 3.43 (s, 2H), 4.14 (d, 2H), 5.54-4.96 (m, 1H), 6.30 (s, 1H), 7.39 (d, 1H), 7.77 (d, 1H), 8.28 (d, 1H), 8.59 (s, 1H), 10.52 (s, 1H).

### EXAMPLE 50

### 3-(4-{[(3R)-1-(3-Hydroxy-3-methylbutanoyl)piperidin-3-yl]amino}pyrimidin-2-yl)imidazo[1,2-a]pyridine-6-carbonitrile

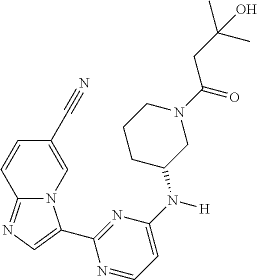

Obtained as an off-white solid (23%) from 3-{4-[(3R)-piperidin-3-ylamino]pyrimidin-2-yl}imidazo[1,2-a]pyridine-6-carbonitrile (Preparation 5b) and 3-hydroxy-3-methyl butanoic acid following the experimental procedure as described in Example 48. The crude product was purified by reverse phase chromatography (C-18 silica from Waters, water/1:1 acetonitrile-methanol as eluents [0.1% v/v formic acid buffered] 0% to 100%).

LRMS (m/z): 420 (M+1)⁺.

¹H-NMR δ (CDCl₃): 1.33 (d, 6H), 1.65-1.97 (m, 4H), 2.14 (bs, 1H), 2.55 (s, 2H), 3.31-3.96 (m, 2H), 5.04-5.18 (m, 2H), 6.29 (d, 1H), 7.40 (d, 1H), 7.80 (t, 1H), 8.30 (d, 1H), 8.56 (s, 1H), 8.62 (s, 1H), 10.54 (s, 1H)

### EXAMPLE 51

### 3-(4-{[(3R)-1-(3,3-Dimethylbutanoyl)piperidin-3-yl]amino}pyrimidin-2-yl)imidazo[1,2-a]pyridine-6-carbonitrile

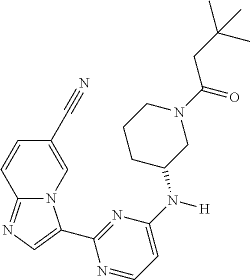

Obtained as an off-white solid from 3-{4-[(3R)-piperidin-3-ylamino]pyrimidin-2-yl}imidazo[1,2-a]pyridine-6-carbonitrile (Preparation 5b) and 3,3-dimethylbutanoic acid following the experimental procedure as described in Example 48. The crude product was purified by reverse phase chromatography (C-18 silica from Waters, water/1:1 acetonitrile-methanol as eluents [0.1% v/v formic acid buffered] 0% to 100%).

LRMS (m/z): 418 (M+1)⁺.

¹H-NMR δ (CDCl₃): 0.99 (s, 9H), 1.71 (bs, 2H), 1.84 (bs, 2H), 2.13 (bs, 2H), 3.38 (bs, 1H), 3.65 (bs, 2H), 3.96 (bs, 2H), 5.14-5.24 (m, 1H), 6.26 (d, 1H), 6.29-6.40 (m, 1H), 7.41 (t, 1H), 7.79 (s, 1H), 8.23-8.35 (m, 1H), 8.61 (s, 1H)

### EXAMPLE 52

### 3-(4-{[(3R)-1-(1H-Imidazol-4-ylacetyl)piperidin-3-yl]amino}pyrimidin-2-yl)imidazo[1,2-a]pyridine-6-carbonitrile

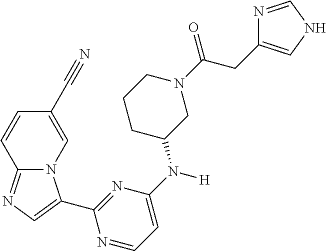

Triethylamine (0.15 mL, 1.1 mmol) and 1,1′-carbonyldiimidazole (0.228 g, 1.4 mmol) were added to a stirred solution of 2-(1H-imidazol-4-yl)acetic acid (0.120 g, 0.95 mmol) in N,N′-dimethylformamide (2 mL). After stirring for 20 minutes at ambient temperature a solution of (R)-3-(4-(piperidin-3-ylamino)pyrimidin-2-yl)imidazo[1,2-a]pyridine-6-carbonitrile (Preparation 5b, 0.150 g, 0.47 mmol) in N,N′-dimethylformamide (2 mL) was added and the reaction mixture was stirred overnight. The solvent was removed under reduced pressure and the residue was purified by reverse phase chromatography (C-18 silica from Waters, water/1:1 acetonitrile-methanol as eluents [0.1% v/v formic acid buffered] 0% to 100%) to give the title compound (0.064 g, 32%) as a solid.

LRMS (m/z): 428 (M+1)⁺.

¹H-NMR δ (DMSO-d₆): 1.55 (bs, 1H), 1.73 (bs, 1H), 2.02 (bs, 2H), 2.52-2.66 (m, 1H), 3.02-3.51 (m, 2H), 3.61 (d, 2H), 3.95 (d, 1H), 4.67 (d, 1H), 6.38-6.55 (m, 1H), 6.87 (bs, 1H), 7.55 (s, 1H), 7.69 (d, 1H), 7.90 (d, 1H), 8.21 (s, 1H), 8.51 (s, 1H), 8.63 (bs, 1H), 10.45 (bs, 1H)

### EXAMPLE 53

### 3-[4-({(3R)-1-[(5-Cyanopyridin-2-yl)carbonyl]piperidin-3-yl}amino)pyrimidin-2-yl]imidazo[1,2-a]pyridine-6-carbonitrile

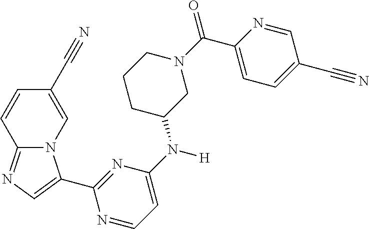

Obtained as an off-white solid (23%) from 3-{4-[(3R)-piperidin-3-ylamino]pyrimidin-2-yl}imidazo[1,2-a]pyridine-6-carbonitrile (Preparation 5b) and 5-cyanopyridine-2-carboxylic acid following the experimental procedure as described in Example 48 with the exception that N,N′-dimethylformamide was used as the reaction solvent. The crude product was purified by reverse phase chromatography (C-18 silica from Waters, water/1:1 acetonitrile-methanol as eluents [0.1% v/v formic acid buffered] 0% to 100%).

LRMS (m/z): 450 (M+1)⁺.

¹H-NMR δ (DMSO-d₆): 1.93 (m, 2H), 3.05-2.77 (m, 1H), 3.57 (d, 1H), 4.23-3.71 (m, 2H), 5.82 (s, 1H), 6.51 (d, 1H), 7.61-7.36 (m, 2H), 7.86 (dd, 2H), 8.60-8.07 (m, 2H), 10.51 (d, 1H).

### EXAMPLE 54

### 3-(4-{[(3R)-1-(3,3,3-Trifluoropropanoyl)piperidin-3-yl]amino}pyrimidin-2-yl)imidazo[1,2-a]pyridine-6-carbonitrile

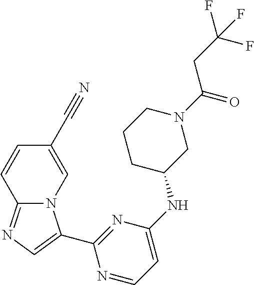 [SMILES: N#Cc1ccc2ncc(-c3nccc(N[C@@H]4CCCN(C(=O)CC(F)(F)F)C4)n3)n2c1]

N-[(Dimethylamino)(3H-[1,2,3]triazolo[4,5-b]pyridin-3-yloxy)methylene]-N-methyl-methanaminium hexafluorophosphate (0.060 g, 0.16 mmol) and diisopropylethylamine (0.028 mL, 0.16 mmol) were added to a stirred solution of 3,3,3-trifluoropropanoic acid (0.015 mL, 0.17 mmol) in N,N′-dimethylformamide (2 mL). The mixture was stirred for 10 minutes at ambient temperature and then 3-{4-[(3R)-piperidin-3-ylamino]pyrimidin-2-yl}imidazo[1,2-a]pyridine-6-carbonitrile (Preparation 5b, 0.050 g, 0.16 mmol) was added. The mixture was stirred overnight and then was partitioned between water and dichloromethane. The organic layer was washed with water, brine, dried (MgSO₄) and evaporated and the residue was purified by flash chromatography (98:2 to 96:4 dichloromethane/methanol) to give the title compound (0.040 g, 60%) as a white solid.

LRMS (m/z): 430 (M+1)⁺.

¹H-NMR δ (CDCl₃): 1.47-1.07 (m, 2H), 1.82 (d, 2H), 2.16 (s, 1H), 3.38 (dt, 2H), 3.93-3.54 (m, 1H), 5.00 (d, 1H), 6.30 (s, 1H), 7.40 (d, 1H), 7.77 (d, 1H), 8.30 (s, 1H), 8.58 (d, 1H), 10.52 (s, 1H).

### EXAMPLE 55

### (3R)-3-{[2-(6-Cyanoimidazo[1,2-a]pyridin-3-yl)pyrimidin-4-yl]amino}-N,N-dimethyl piperidine-1-carboxamide

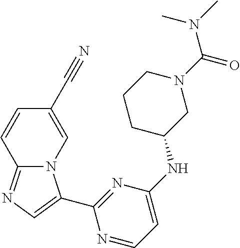 [SMILES: CN(C)C(=O)N1CCC[C@@H](Nc2ccnc(-c3cnc4ccc(C#N)cn34)n2)C1]

Triethylamine (0.019 mL, 0.14 mmol) and dimethylcarbamic chloride (0.017 mL, 0.18 mmol) were added to a stirred solution of 3-{4-[(3R)-piperidin-3-ylamino]pyrimidin-2-yl}imidazo[1,2-a]pyridine-6-carbonitrile (Preparation 5b, 0.040 g, 0.13 mmol) in dichloromethane (1 mL). The mixture was stirred at ambient temperature for 2 hours then the mixture was diluted with dichloromethane and washed with water, brine, dried (MgSO₄) and evaporated. The solid was dissolved in 2M aqueous hydrochloric acid and washed with ethyl acetate. The aqueous layer was basified with 2M aqueous sodium hydroxide solution and the product was extracted into dichloromethane. The organic layer was dried (MgSO₄) and evaporated to obtain the title compound (0.032 g, 65%) as a pale yellow solid.

LRMS (m/z): 391 (M+1)⁺.

¹H-NMR δ (CDCl₃): 1.61 (bs, 1H), 1.85 (d, 1H), 2.89 (s, 6H), 3.31 (bs, 4H), 6.34 (bs, 1H), 7.43-7.32 (m, 1H), 7.77 (d, 1H), 8.20 (s, 1H), 8.58 (s, 1H), 10.59 (s, 1H).

### EXAMPLE 56

### 3-{4-[((3R)-1-{[(2S,4S)-2-Cyano-4-fluoropyrrolidin-1-yl]carbonyl}piperidin-3-yl)amino]pyrimidin-2-yl}imidazo[1,2-a]pyridine-6-carbonitrile

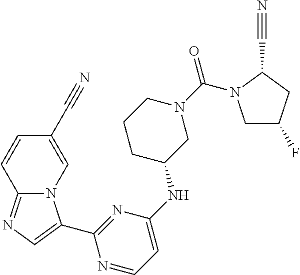 [SMILES: N#Cc1ccc2ncc(-c3nccc(N[C@@H]4CCCN(C(=O)N5C[C@@H](F)C[C@H]5C#N)C4)n3)n2c1]

A solution of 1-((2S,4S)-2-cyano-4-fluoropyrrolidine-1-carbonyl)-3-methyl-1H-imidazol-3-ium iodide (Preparation 36, 0.100 g, 0.29 mmol), (R)-3-(4-(piperidin-3-ylamino)pyrimidin-2-yl)imidazo[1,2-a]pyridine-6-carbonitrile (Preparation 5b, 94 mg, 0.29 mmol) and triethylamine (0.075 mL, 0.54 mmol) in a mixture of dichloromethane (2 mL) and N,N′-dimethylformamide (1 mL) was stirred at ambient temperature overnight. The solvent was removed under reduced pressure and the residue was purified by reverse phase chromatography (C-18 silica from Waters, water/acetonitrile/methanol as eluents [0.1% v/v formic acid buffered] 0% to 100%) to obtain the title compound (0.022 g, 17%) as a solid.

LRMS (m/z): 460 (M+1)⁺.

¹H-NMR δ (CDCl₃): 1.62-2.13 (m, 4H), 2.24-2.44 (m, 1H), 2.56 (bs, 1H), 3.29 (bs, 1H), 3.55 (bs, 1H), 3.66-3.95 (m, 3H), 5.09 (bs, 1H), 5.21-5.43 (m, 2H), 6.36 (bs, 1H), 7.40 (d, 1H), 7.76 (d, 1H), 8.25 (bs, 1H), 8.56 (s, 1H), 10.53 (s, 1H)

### EXAMPLE 57

### 3-(4-{[(3R)-1-(5-Cyanopyridin-2-yl)piperidin-3-yl]amino}pyrimidin-2-yl)imidazo[1,2-a]pyridine-6-carbonitrile

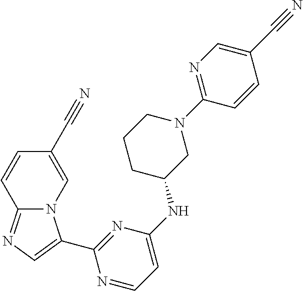 [SMILES: N#Cc1ccc(N2CCC[C@@H](Nc3ccnc(-c4cnc5ccc(C#N)cn45)n3)C2)nc1]

Triethylamine (0.020 mL, 0.14 mmol) and 6-chloronicotinonitrile (0.050 g, 0.37 mmol) were added to a stirred solution of 3-{4-[(3R)-piperidin-3-ylamino]pyrimidin-2-yl}imidazo[1,2-a]pyridine-6-carbonitrile (Preparation 5b, 0.045 g, 0.14 mmol) in dichloromethane (2 mL). The mixture was heated to 50° C. in a sealed tube and stirred overnight. The mixture was partitioned between dichloromethane and water and the organic layer was washed with water, brine, dried (MgSO₄) and evaporated. The residue was purified by flash chromatography (98:2 dichloromethane/methanol) to give a solid that was dissolved in 2M aqueous hydrochloric acid (2.0 mL) and washed with ethyl acetate. The aqueous layer was basified with 2M aqueous sodium hydroxide solution and the product was extracted with dichloromethane. The organic layer was dried (MgSO₄) and evaporated to obtain the title compound (0.018 g, 30%) as a white solid.

LRMS (m/z): 422 (M+1)⁺.

¹H-NMR δ (CDCl₃): 2.26-1.65 (m, 4H), 3.48-3.27 (m, 2H), 3.99 (dt, 2H), 4.51 (d, 1H), 5.12 (s, 1H), 6.31 (bs, 1H), 6.69 (d, 1H), 7.41 (dd, 1H), 7.63 (dd, 1H), 7.81 (d, 1H), 8.30 (d, 1H), 8.41 (d, 1H), 8.62 (s, 1H), 10.54 (s, 1H).

### EXAMPLE 58

### 3-(4-{[(3R)-1-(4-Cyano-2-fluorophenyl)piperidin-3-yl]amino}pyrimidin-2-yl)imidazo[1,2-a]pyridine-6-carbonitrile

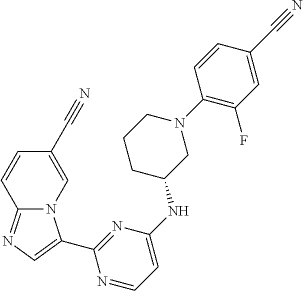 [SMILES: N#Cc1ccc(N2CCC[C@@H](Nc3ccnc(-c4cnc5ccc(C#N)cn45)n3)C2)c(F)c1]

3,4-Difluorobenzonitrile (0.022 g, 0.16 mmol) and potassium carbonate (0.025 g, 0.18 mmol) were added to a stirred solution of 3-{4-[(3R)-piperidin-3-ylamino]pyrimidin-2-yl}imidazo[1,2-a]pyridine-6-carbonitrile (Preparation 5b, 0.040 g, 0.13 mmol) in N,N′-dimethylformamide (1 mL). The mixture was heated to 80° C. in a sealed tube and stirred overnight. The mixture was evaporated and the residue was purified by flash chromatography (98:2 dichloromethane/methanol) and the solid obtained was triturated with diethyl ether to give the title compound (0.020 g, 36%) as a white solid.

LRMS (m/z): 439 (M+1)⁺.

¹H-NMR δ (CDCl₃): 2.06-1.79 (m, 4H), 3.21 (bs, 3H), 3.56-3.45 (m, 2H), 5.38 (s, 1H), 6.31 (d, 1H), 7.01 (t, 3H), 7.34-7.28 (m, 1H), 7.44-7.36 (m, 2H), 7.79 (d, 1H), 8.29 (d, 1H), 8.60 (s, 1H), 10.56 (s, 1H).

### EXAMPLE 59

### 3-{4-[[(3R)-1-(Cyanoacetyl)piperidin-3-yl](methyl)amino]pyrimidin-2-yl}imidazo[1,2-a]pyridine-6-carbonitrile

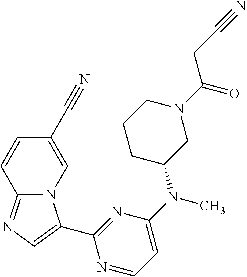 [SMILES: CN(c1ccnc(-c2cnc3ccc(C#N)cn23)n1)[C@@H]1CCCN(C(=O)CC#N)C1]

3-[(2,5-Dioxopyrrolidin-1-yl)oxy]-3-oxopropanenitrile (prepared as described in BE875054(A1), 0.020 g, 0.11 mmol) was added to a stirred solution of (R)-3-(4-(methyl(piperidin-3-yl)amino)pyrimidin-2-yl)imidazo[1,2-a]pyridine-6-carbonitrile (Preparation 6b, 0.030 g, 0.09 mmol) in ethanol (2 mL). The mixture was stirred at ambient temperature for 48 hours and then was evaporated. Water was added and the mixture was extracted with dichloromethane. The organic layer was dried (MgSO₄), evaporated and the residue was purified by reverse phase chromatography (C-18 silica from Waters, water/1:1 acetonitrile-methanol as eluents [0.1% v/v formic acid buffered] 0% to 100%) to give the title compound (0.013 g, 36%) as a solid.

LRMS (m/z): 401 (M+1)⁺.

¹H-NMR δ (CDCl₃): 1.90 (bs, 2H), 2.07 (bs, 2H), 2.73 (bs, 1H), 3.05 (bs, 3H), 3.11-3.24 (m, 1H), 3.68 (bs, 2H), 3.78 (bs, 1H), 4.74 (bs, 2H), 6.38 (bs, 1H), 7.39 (bs, 1H), 7.78 (bs, 1H), 8.35 (bs, 1H), 8.64 (bs, 1H), 10.43 (bs, 1H)

### EXAMPLE 60

### 3-(4-{[(3S)-1-(Cyanoacetyl)piperidin-3-yl]amino}pyrimidin-2-yl)imidazo[1,2-a]pyridine-6-carbonitrile

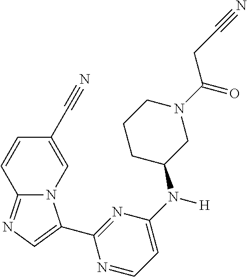 [SMILES: N#CCC(=O)N1CCC[C@H](Nc2ccnc(-c3cnc4ccc(C#N)cn34)n2)C1]

Obtained as a off-white solid (62%) from 3-{4-[(3S)-piperidin-3-ylamino]pyrimidin-2-yl}imidazo[1,2-a]pyridine-6-carbonitrile (Preparation 7b) and 3-[(2,5-dioxopyrrolidin-1-yl)oxy]-3-oxopropanenitrile (prepared as described in BE875054(A1)) following the experimental procedure as described in Example 46. The crude product was purified by flash chromatography (97:3 to 96:4 dichloromethane/methanol).

LRMS (m/z): 387 (M+1)⁺.

¹H-NMR δ (DMSO-d₆): 4.11 (s, 2H), 4.72 (d, 1H), 6.49 (d, 1H), 7.75 (d, 2H), 7.96 (d, 1H), 8.29 (s, 1H), 8.66 (s, 1H), 10.50 (s, 1H).

### EXAMPLE 61

### Cis-3-(4-{1-(cyanoacetyl)-4-methylpiperidin-3-yl]amino}pyrimidin-2-yl)imidazo[1,2-a]pyridine-6-carbonitrile

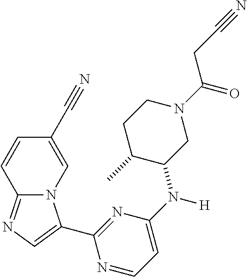

Obtained as a white solid (26%) from 3-(4-hydroxypyrimidin-2-yl)imidazo[1,2-a]pyridine-6-carbonitrile (Preparation 4b) and cis-3-(3-amino-4-methylpiperidin-1-yl)-3-oxopropanenitrile (Preparation 34d) following the experimental procedure as described in Preparation 5a followed by heating the reaction to 40° C. for 48 hours. After the reaction was complete the mixture was partitioned between water and ethyl acetate. The organic layer was washed with water, saturated aqueous sodium hydrogen carbonate solution, brine, dried (MgSO₄) and evaporated and the residue was purified by reverse phase chromatography (C-18 silica from Waters, water/acetonitrile/methanol as eluents [0.1% v/v formic acid buffered] 0% to 100%).

LRMS (m/z): 401 (M+1)⁺.

¹H-NMR δ (CDCl₃): 1.07 (t, 3H), 2.26-2.11 (m, 1H), 3.31 (d, 2H), 3.58 (d, 1H), 4.01 (d, 1H), 4.43 (d, 1H), 4.63 (d, 1H), 5.06-4.93 (m, 1H), 6.35 (dd, 1H), 7.49-7.35 (m, 1H), 7.80 (dd, 1H), 8.29 (dd, 1H), 8.57 (s, 1H), 10.56 (d, 1H).

### EXAMPLE 62

### Cis-3-(4-{1-(ethylsulfonyl)-4-methylpiperidin-3-yl]amino}pyrimidin-2-yl)imidazo[1,2-a]pyridine-6-carbonitrile

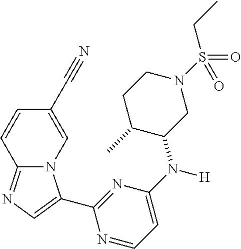

Obtained as a pale yellow solid (44%) from 3-(4-hydroxypyrimidin-2-yl)imidazo[1,2-a]pyridine-6-carbonitrile (Preparation 4b) and cis-1-(ethylsulfonyl)-4-methylpiperidin-3-amine (Preparation 35b) following the experimental procedure as described in Preparation 5a followed by heating the reaction to 45° C. for 18 hours. After the reaction was complete the mixture was partitioned between water and ethyl acetate. The organic layer was washed with water, saturated aqueous sodium hydrogen carbonate solution, brine, dried (MgSO₄) and evaporated and the residue was purified by flash chromatography (98:2 dichloromethane/methanol).

LRMS (m/z): 426 (M+1)⁺.

¹H-NMR δ (CDCl₃): 1.04 (d, 3H), 1.38 (t, 3H), 1.75-1.52 (bs, 2H), 2.08-1.83 (m, 2H), 3.17-2.81 (m, 4H), 3.89 (d, 2H), 4.61 (s, 1H), 5.48 (d, 1H), 6.35 (d, 1H), 7.40 (dd, 1H), 7.77 (d, 1H), 8.22 (d, 1H), 8.55 (s, 1H), 10.54 (s, 1H).

### EXAMPLE 63

### 3-{4-[[1-(Ethylsulfonyl)-4-methylpiperidin-3-yl](methyl)amino]pyrimidin-2-yl}imidazo[1,2-a]pyridine-6-carbonitrile

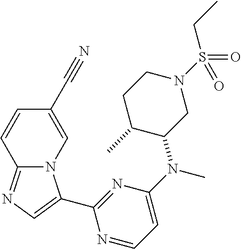

Obtained as a pale yellow solid (31%) from 3-(4-hydroxypyrimidin-2-yl)imidazo[1,2-a]pyridine-6-carbonitrile (Preparation 4b) and 1-(ethylsulfonyl)-N,3-dimethylpiperidin-4-amine (Preparation 33d) following the experimental procedure as described in Preparation 5a followed by heating the reaction to 50° C. for 96 hours. The crude product was purified by flash chromatography (97:3 dichloromethane/methanol) and triturated with methanol to give the pure title compound.

LRMS (m/z): 440 (M+1)⁺.

¹H-NMR δ (DMSO-d₆): 1.03 (d, 3H), 1.24 (bs, 3H), 1.72 (bs, 2H), 1.91-2.05 (m, 1H), 2.51 (bs, 3H), 3.10-3.21 (m, 1H), 3.23-3.36 (m, 4H), 3.40-3.40 (m, 2H), 7.86-7.93 (m, 1H), 8.00-8.07 (m, 1H), 8.41 (d, 1H), 8.85 (bs, 1H), 10.19 (bs, 1H)

### EXAMPLE 64

### 3-((3R)-3-{[2-(6-Fluoroimidazo[1,2-a]pyridin-3-yl)pyrimidin-4-yl]amino}piperidin-1-yl)-3-oxopropanenitrile

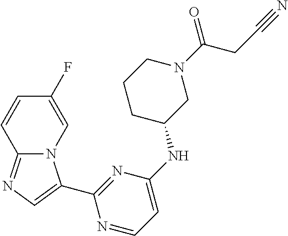 [SMILES: N#CCC(=O)N1CCC[C@@H](Nc2ccnc(-c3cnc4ccc(F)cn34)n2)C1]

Obtained as an off-white solid (98%) from 2-(6-fluoroimidazo[1,2-a]pyridin-3-yl)-N-(piperidin-3-yl)pyrimidin-4-amine (Preparation 9b) and 3-[(2,5-dioxopyrrolidin-1-yl)oxy]-3-oxopropanenitrile (prepared as described in BE875054(A1)) following the experimental procedure as described in Example 46. The crude product was purified by reverse phase chromatography (C-18 silica from Waters, water/1:1 acetonitrile-methanol as eluents [0.1% v/v formic acid buffered] 0% to 100%).

LRMS (m/z): 380 (M+1)⁺.

¹H-NMR δ (CDCl₃): 1.80 (bs, 3H), 1.97 (bs, 1H), 2.16 (bs, 2H), 3.57 (s, 2H), 3.67 (bs, 2H), 5.00 (s, 1H), 6.28 (dd, 1H), 7.30-7.19 (m, 1H), 7.69 (td, 1H), 8.29 (t, 1H), 8.48 (s, 1H), 8.55 (s, 1H), 9.96 (ddd, 1H).

### EXAMPLE 65

### (R)-1-(3-(2-(6-Fluoroimidazo[1,2-a]pyridin-3-yl)pyrimidin-4-ylamino)piperidine-1-carbonyl)cyclopropanecarbonitrile

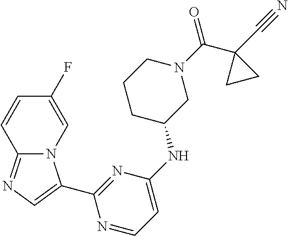 [SMILES: N#CC1(C(=O)N2CCC[C@@H](Nc3ccnc(-c4cnc5ccc(F)cn45)n3)C2)CC1]

Obtained as a white solid (76%) from (R)-2-(6-fluoroimidazo[1,2-a]pyridin-3-yl)-N-(piperidin-3-yl)pyrimidin-4-amine (Preparation 9b) and 1-cyanocyclopropanecarboxylic acid following the experimental procedure as described in Example 48. The crude product was purified by flash chromatography (100:8 dichloromethane/methanol).

LRMS (m/z): 406 (M+1)⁺.

¹H-NMR δ (CDCl₃): 1.56 (s, 3H), 1.90 (dd, 2H), 2.19 (d, 2H), 4.34-4.16 (m, 2H), 4.96 (s, 1H), 6.28 (s, 1H), 7.23 (dd, 1H), 7.68 (dd, 1H), 8.29 (d, 1H), 8.55 (s, 1H), 9.96 (d, 1H).

### EXAMPLE 66

### 2-(6-Fluoroimidazo[1,2-a]pyridin-3-yl)-N-[(3R)-1-(3,3,3-trifluoropropanoyl)piperidin-3-yl]pyrimidin-4-amine

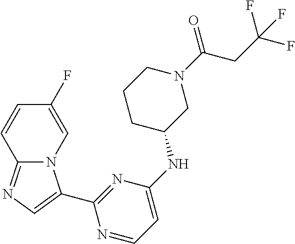 [SMILES: O=C(CC(F)(F)F)N1CCC[C@@H](Nc2ccnc(-c3cnc4ccc(F)cn34)n2)C1]

Obtained as a white solid (59%) from (R)-2-(6-fluoroimidazo[1,2-a]pyridin-3-yl)-N-(piperidin-3-yl)pyrimidin-4-amine (Preparation 9b) and 3,3,3-trifluoropropanoic acid following the experimental procedure as described in Example 54. The crude product was purified by reverse phase chromatography (C-18 silica from Waters, water/1:1 acetonitrile-methanol as eluents [0.1% v/v formic acid buffered] 0% to 100%).

LRMS (m/z): 423 (M+1)⁺.

¹H-NMR δ (CDCl₃): 1.25 (s, 2H), 1.73 (bs, 2H), 1.90 (bs, 2H), 2.15 (bs, 2H), 3.42 (bs, 2H), 6.22 (d, 1H), 6.27 (d, 1H), 7.25-7.19 (m, 1H), 7.68 (td, 1H), 8.29 (t, 1H), 8.49 (s, 1H), 8.53 (s, 1H), 9.97 (s, 1H).

### EXAMPLE 67

### 2-(6-Fluoroimidazo[1,2-a]pyridin-3-yl)-N-[(3R)-1-(1H-pyrazol-4ylcarbonyl)piperidin-3-yl]pyrimidin-4-amine

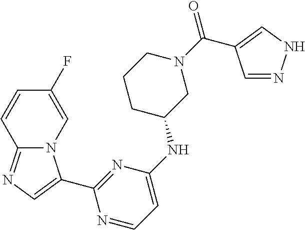 [SMILES: O=C(c1cn[nH]c1)N1CCC[C@@H](Nc2ccnc(-c3cnc4ccc(F)cn34)n2)C1]

Obtained as a solid (33%) from (R)-2-(6-fluoroimidazo[1,2-a]pyridin-3-yl)-N-(piperidin-3-yl)pyrimidin-4-amine (Preparation 9b) and 1H-pyrazole-4-carboxylic acid following the experimental procedure as described in Example 52. The crude product was purified by reverse phase chromatography (C-18 silica from Waters, water/1:1 acetonitrile-methanol as eluents [0.1% v/v formic acid buffered] 0% to 100%).

LRMS (m/z): 407 (M+1)⁺.

¹H-NMR δ (CDCl₃): 2.65 (d, 4H), 3.21-3.24 (m, 2H), 3.50 (d, 2H), 3.79 (d, 2H), 6.27 (d, 1H), 7.00 (bs, 1H), 7.23-7.29 (m, 1H), 7.71 (dd, 1H), 7.91 (s, 1H), 8.28 (d, 1H), 8.47 (s, 1H), 9.97 (dd, 1H)

### EXAMPLE 68

### (3R)—N-(Cyanomethyl)-3-{[2-(6-fluoroimidazo[1,2-a]pyridin-3-yl)pyrimidin-4-yl]amino}-N-methylpiperidine-1-carboxamide

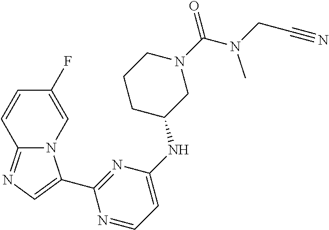 [SMILES: CN(CC#N)C(=O)N1CCC[C@@H](Nc2ccnc(-c3cnc4ccc(F)cn34)n2)C1]

Obtained as a solid (33%) from (R)-2-(6-fluoroimidazo[1,2-a]pyridin-3-yl)-N-(piperidin-3-yl)pyrimidin-4-amine (Preparation 9b) and 1-((cyanomethyl)(methyl)carbamoyl)-3-methyl-1H-imidazol-3-ium iodide (Preparation 37b) following the experimental procedure as described in Example 56. The crude product was purified by reverse phase chromatography (C-18 silica from Waters, water/1:1 acetonitrile-methanol as eluents [0.1% v/v formic acid buffered] 0% to 100%).

LRMS (m/z): 409 (M+1)⁺.

¹H-NMR δ (CDCl₃): 1.67 (bs, 1H), 1.83 (bs, 1H), 2.01 (bs, 1H), 2.26 (bs, 2H), 3.00 (s, 3H), 3.30 (bs, 1H), 3.44 (bs, 2H), 3.66 (bs, 1H), 3.96-4.17 (m, 2H), 5.75 (bs, 1H), 6.28 (bs, 1H), 7.18-7.32 (m, 1H), 7.71 (dd, 1H), 8.26 (bs, 1H), 8.50 (s, 1H), 10.00 (dd, 1H)

### EXAMPLE 69

### 2-((3R)-3-{[2-(6-Fluoroimidazo[1,2-a]pyridin-3-yl)pyrimidin-4-yl]amino}piperidin-1-yl)-2-methylpropanamide

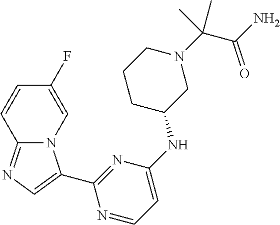 [SMILES: CC(C)(C(N)=O)N1CCC[C@@H](Nc2ccnc(-c3cnc4ccc(F)cn34)n2)C1]

Concentrated aqueous ammonium hydroxide solution (0.01 mL) was added to a stirred solution of (R)-2,5-dioxopyrrolidin-1-yl 2-(3-(2-(6-fluoroimidazo[1,2-a]pyridin-3-yl)pyrimidin-4-ylamino)piperidin-1-yl)-2-methylpropanoate (Preparation 10c) in 1,4-dioxane (1 mL) and the mixture was stirred at ambient temperature overnight. The reaction mixture was diluted with water and acidified with 2M aqueous hydrochloric acid and extracted with dichloromethane. The organic phase was washed with 4% aqueous sodium hydrogen carbonate solution and brine. The aqueous phase was extracted with further dichloromethane and the combined organic phase was dried (MgSO₄), evaporated and the residue was purified by reverse phase chromatography (C-18 silica from Waters, water/1:1 acetonitrile-methanol as eluents [0.1% v/v formic acid buffered] 0% to 100%) to obtain the title compound (0.009 g, 33%) as a solid.

LRMS (m/z): 398 (M+1)⁺.

¹H-NMR δ (CDCl₃): 1.15 (d, 6H), 1.90 (bs, 4H), 2.01-2.14 (m, 2H), 2.28-2.45 (m, 2H), 2.74 (bs, 1H), 3.76-3.87 (m, 2H), 5.53 (bs, 1H), 6.20 (d, 1H), 7.27 (dd, 1H), 7.72 (dd, 1H), 8.26 (bs, 1H), 8.52 (s, 1H), 10.01 (dd, 1H)

### EXAMPLE 70

### 2-(6-Fluoroimidazo[1,2-a]pyridin-3-yl)-N-methyl-N-[(3R)-1-(3,3,3-trifluoro propanoyl)piperidin-3-yl]pyrimidin-4-amine

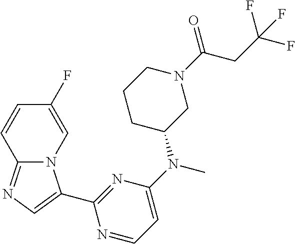 [SMILES: CN(c1ccnc(-c2cnc3ccc(F)cn23)n1)[C@@H]1CCCN(C(=O)CC(F)(F)F)C1]

Obtained as a white solid (83%) from (R)-2-(6-fluoroimidazo[1,2-a]pyridin-3-yl)-N-methyl-N-(piperidin-3-yl)pyrimidin-4-amine (Preparation 11b) and 3,3,3-trifluoro propanoic acid following the experimental procedure as described in Example 54. The crude product was purified by flash chromatography (0-5% methanol in dichloromethane).

LRMS (m/z): 437 (M+1)⁺.

¹H-NMR δ (CDCl₃): 1.83-2.14 (m, 4H), 3.05 (s, 2H), 3.07 (s, 3H), 3.12-3.41 (m, 3H), 3.78 (bs, 2H), 6.36 (t, 1H), 7.24 (dd, 1H), 7.70 (dt, 1H), 8.30-8.40 (m, 1H), 8.53 (s, 1H), 9.91 (dd, 1H)

### EXAMPLE 71

### (R)-3-(3-((2-(6-Fluoroimidazo[1,2-a]pyridin-3-yl)pyrimidin-4-yl)(methyl)amino) piperidin-1-yl)-3-oxopropanenitrile

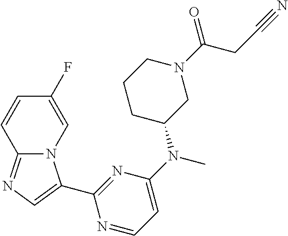 [SMILES: CN(c1ccnc(-c2cnc3ccc(F)cn23)n1)[C@@H]1CCCN(C(=O)CC#N)C1]

Obtained as a white solid (77%) from (R)-2-(6-fluoroimidazo[1,2-a]pyridin-3-yl)-N-methyl-N-(piperidin-3-yl)pyrimidin-4-amine (Preparation 11b) and 3-[(2,5-dioxopyrrolidin-1-yl)oxy]-3-oxopropanenitrile (prepared as described in BE875054(A1)) following the experimental procedure as described in Example 46. The crude product was purified by flash chromatography (0-5% methanol in dichloromethane).

LRMS (m/z): 394 (M+1)⁺.

¹H-NMR δ (CDCl₃): 1.71-2.14 (m, 4H), 2.57-2.68 (m, 1H), 2.80 (s, 3H), 3.06 (s, 2H), 3.13-3.35 (m, 2H), 3.71-3.86 (m, 2H), 4.62-4.75 (m, 1H), 6.37 (dd, 1H), 7.24 (dt, 1H), 7.68 (dt, 1H), 8.31-8.40 (m, 1H), 8.54 (s, 1H), 9.86 (dd, 1H)

### EXAMPLE 72

### (R)-1-(3-((2-(6-Fluoroimidazo[1,2-a]pyridin-3-yl)pyrimidin-4-yl)(methyl)amino) piperidine-1-carbonyl)cyclopropanecarbonitrile

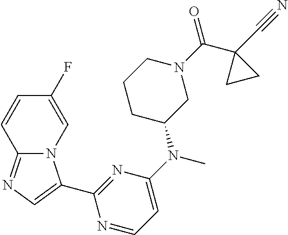 [SMILES: CN(c1ccnc(-c2cnc3ccc(F)cn23)n1)[C@@H]1CCCN(C(=O)C2(C#N)CC2)C1]

Obtained as a white solid (47%) from (R)-2-(6-fluoroimidazo[1,2-a]pyridin-3-yl)-N-methyl-N-(piperidin-3-yl)pyrimidin-4-amine (Preparation 11b) and 1-cyanocyclopropane carboxylic acid following the experimental procedure as described in Example 48. The crude product was purified by flash chromatography (0-8% methanol in dichloromethane) followed by trituration with diethyl ether.

LRMS (m/z): 420 (M+1)⁺.

¹H-NMR δ (CDCl₃): 1.55 (bs, 4H), 1.90-2.14 (m, 4H), 2.80-2.94 (m, 1H), 3.08 (s, 3H), 3.15-3.31 (m, 2H), 4.53 (d, 2H), 6.39 (bs, 1H), 7.23 (dd, 1H), 7.68 (dd, 1H), 8.33 (d, 1H), 8.51 (bs, 1H), 9.91 (bs, 1H)

### EXAMPLE 73

### 3-((3R)-3-{Ethyl[2-(6-fluoroimidazo[1,2-a]pyridin-3-yl)pyrimidin-4-yl]amino}piperidin-1-yl)-3-oxopropanenitrile

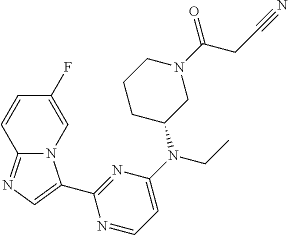 [SMILES: CCN(c1ccnc(-c2cnc3ccc(F)cn23)n1)[C@@H]1CCCN(C(=O)CC#N)C1]

Obtained as a white solid (77%) from N-ethyl-2-(6-fluoroimidazo[1,2-a]pyridin-3-yl)-N-[(3R)-piperidin-3-yl]pyrimidin-4-amine (Preparation 12b) and 3-[(2,5-dioxopyrrolidin-1-yl)oxy]-3-oxopropanenitrile (prepared as described in BE875054(A1)) following the experimental procedure as described in Example 46. The crude product was purified by flash chromatography (92:8 dichloromethane/methanol).

LRMS (m/z): 408 (M+1)⁺.

¹H-NMR δ (CDCl₃): 1.30 (bs, 3H), 1.77 (bs, 2H), 2.02 (bs, 2H), 2.84 (d, 1H), 3.18 (bs, 2H), 3.39-3.59 (m, 4H), 3.79 (bs, 1H), 4.72 (bs, 1H), 6.33 (bs, 1H), 7.23 (bs, 1H), 7.68 (bs, 1H), 8.32 (bs, 1H), 8.37-8.55 (m, 1H), 9.80-10.01 (m, 1H)

### EXAMPLE 74

### N-Ethyl-2-(6-fluoroimidazo[1,2-a]pyridin-3-yl)-N-[(3R)-1-(3,3,3-trifluoropropanoyl)piperidin-3-yl]pyrimidin-4-amine

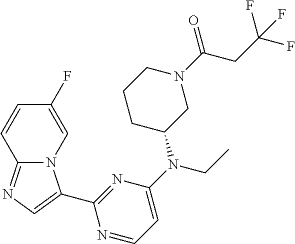 [SMILES: CCN(c1ccnc(-c2cnc3ccc(F)cn23)n1)[C@@H]1CCCN(C(=O)CC(F)(F)F)C1]

Obtained as a solid (52%) from N-ethyl-2-(6-fluoroimidazo[1,2-a]pyridin-3-yl)-N-[(3R)-piperidin-3-yl]pyrimidin-4-amine (Preparation 12b) and 3,3,3-trifluoropropanoic acid following the experimental procedure as described in Example 54. The crude product was purified by flash chromatography (92:8 dichloromethane/methanol).

LRMS (m/z): 451 (M+1)⁺.

¹H-NMR δ (CDCl₃): 1.30 (t, 3H), 1.92-2.18 (m, 2H), 2.51-2.66 (m, 1H), 2.82 (bs, 1H), 3.14 (s, 2H), 3.19-3.28 (m, 1H), 3.32 (q, 2H), 3.51 (bs, 2H), 3.74-3.92 (m, 1H), 4.79 (t, 1H), 6.34 (d, 1H), 7.23 (d, 1H), 7.68 (dt, 1H), 8.29-8.36 (m, 1H), 8.42-8.54 (m, 1H), 9.88-9.99 (m, 1H)

### EXAMPLE 75

### 3-((3R)-3-{(Cyclopropylmethyl)[2-(6-fluoroimidazo[1,2-a]pyridin-3-yl)pyrimidin-4-yl]amino}piperidin-1-yl)-3-oxopropanenitrile

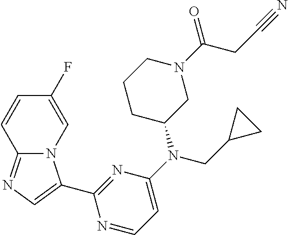 [SMILES: N#CCC(=O)N1CCC[C@@H](N(CC2CC2)c2ccnc(-c3cnc4ccc(F)cn34)n2)C1]

Obtained as a white solid (77%) from N-(cyclopropylmethyl)-2-(6-fluoroimidazo[1,2-a]pyridin-3-yl)-N-[(3R)-piperidin-3-yl]pyrimidin-4-amine (Preparation 13b) and 3-[(2,5-dioxopyrrolidin-1-yl)oxy]-3-oxopropanenitrile (prepared as described in BE875054(A1)) following the experimental procedure as described in Example 46. The crude product was purified by flash chromatography (0-8% methanol in dichloromethane).

LRMS (m/z): 434 (M+1)⁺.

¹H-NMR δ (CDCl₃): 0.39 (bs, 2H), 0.67 (s, 2H), 1.08 (bs, 1H), 1.95-2.18 (m, 4H), 2.97 (bs, 1H), 3.14-3.36 (m, 2H), 3.21 (s, 2H), 3.57 (s, 2H), 3.79 (bs, 1H), 4.66-4.85 (m, 1H), 6.48 (dd, 1H), 7.18-7.30 (m, 1H), 7.68 (dd, 1H), 8.27-8.38 (m, 1H), 8.51 (s, 1H), 9.92 (dd, 1H)

### EXAMPLE 76

### N-(Cyclopropylmethyl)-2-(6-fluoroimidazo[1,2-a]pyridin-3-yl)-N-[(3R)-1-(3,3,3-trifluoropropanoyl)piperidin-3-yl]pyrimidin-4-amine

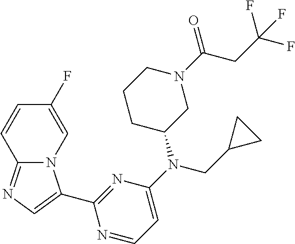 [SMILES: O=C(CC(F)(F)F)N1CCC[C@@H](N(CC2CC2)c2ccnc(-c3cnc4ccc(F)cn34)n2)C1]

Obtained as a white solid (75%) from N-(cyclopropylmethyl)-2-(6-fluoroimidazo[1,2-a]pyridin-3-yl)-N-[(3R)-piperidin-3-yl]pyrimidin-4-amine (Preparation 13b) and 3,3,3-trifluoropropanoic acid following the experimental procedure as described in Example 54.

LRMS (m/z): 477 (M+1)⁺.

¹H-NMR δ (CDCl₃): 0.38 (d, 2H), 0.65 (d, 2H), 1.11 (bs, 1H), 1.46 (d, 2H), 1.92-2.19 (m, 2H), 2.92 (bs, 1H), 3.09-3.28 (m, 3H), 3.47 (bs, 2H), 3.86 (bs, 1H), 4.72-4.91 (m, 2H), 6.47 (t, 1H), 7.24 (dd, 1H), 7.68 (dd, 1H), 8.27-8.38 (m, 1H), 8.51 (s, 1H), 9.97 (ddd, 1H)

### EXAMPLE 77

### 3-((3R)-3-{[5-Fluoro-2-(6-fluoroimidazo[1,2-a]pyridin-3-yl)pyrimidin-4-yl]amino}piperidin-1-yl)-3-oxopropanenitrile

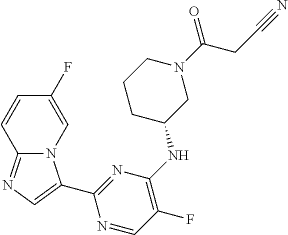 [SMILES: N#CCC(=O)N1CCC[C@@H](Nc2nc(-c3cnc4ccc(F)cn34)ncc2F)C1]

Obtained as a white solid (67%) from (R)-5-fluoro-2-(6-fluoroimidazo[1,2-a]pyridin-3-yl)-N-(piperidin-3-yl)pyrimidin-4-amine (Preparation 17b) and 3-[(2,5-dioxopyrrolidin-1-yl)oxy]-3-oxopropanenitrile (prepared as described in BE875054(A1)) following the experimental procedure as described in Example 46. The crude product was purified by flash chromatography (0-8% methanol in dichloromethane).

LRMS (m/z): 398 (M+1)⁺.

¹H-NMR δ (CDCl₃): 1.73-2.00 (m, 2H), 2.21 (bs, 2H), 3.29-3.39 (m, 1H), 3.45 (s, 2H), 3.48-3.68 (m, 2H), 4.24-4.41 (m, 2H), 5.18 (bs, 1H), 7.68 (ddd, 1H), 8.16 (d, 1H), 8.40 (s, 1H), 8.51 (s, 1H), 9.80-9.91 (m, 1H)

### EXAMPLE 78

### 5-Fluoro-2-(6-fluoroimidazo[1,2-a]pyridin-3-yl)-N-[(3R)-1-(3,3,3-trifluoropropanoyl)piperidin-3-yl]pyrimidin-4-amine

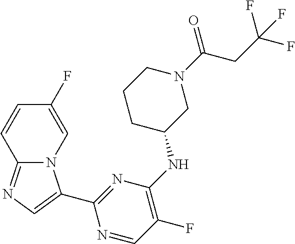 [SMILES: O=C(CC(F)(F)F)N1CCC[C@@H](Nc2nc(-c3cnc4ccc(F)cn34)ncc2F)C1]

Obtained as a white solid (63%) from (R)-5-fluoro-2-(6-fluoroimidazo[1,2-a]pyridin-3-yl)-N-(piperidin-3-yl)pyrimidin-4-amine (Preparation 17b) and 3,3,3-trifluoropropanoic acid following the experimental procedure as described in Example 54. The crude product was purified by flash chromatography (0-8% methanol in dichloromethane).

LRMS (m/z): 441 (M+1)⁺.

¹H-NMR δ (CDCl₃): 1.69-2.01 (m, 3H), 2.18 (bs, 1H), 3.16-3.27 (m, 1H), 3.19-3.46 (m, 2H), 3.48 (d, 1H), 3.54-3.65 (m, 1H), 3.89-4.23 (m, 1H), 4.26-4.37 (m, 1H), 5.04-5.27 (m, 1H), 7.25 (ddd, 1H), 7.69 (td, 1H), 8.16 (dd, 1H), 8.37-8.54 (m, 1H), 9.87 (ddd, 1H)

### EXAMPLE 79

### 3-((3R)-3-{[2-(6-Fluoroimidazo[1,2-a]pyridin-3-O-5-methylpyrimidin-4-yl]amino}piperidin-1-yl)-3-oxopropanenitrile

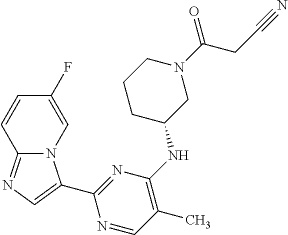 [SMILES: Cc1cnc(-c2cnc3ccc(F)cn23)nc1N[C@@H]1CCCN(C(=O)CC#N)C1]

Obtained as a white solid (66%) from 2-(6-fluoroimidazo[1,2-a]pyridin-3-yl)-5-methyl-N-[(3R)-piperidin-3-yl]pyrimidin-4-amine (Preparation 20b) and 3-[(2,5-dioxopyrrolidin-1-yl)oxy]-3-oxopropanenitrile (prepared as described in BE875054(A1)) following the experimental procedure as described in Example 46. The crude product was purified by flash chromatography (0-8% methanol in dichloromethane).

LRMS (m/z): 394 (M+1)⁺.

¹H-NMR δ (CDCl₃): 1.82 (bs, 2H), 2.09 (bs, 5H), 3.40 (bs, 2H), 3.72-3.88 (m, 1H), 4.17 (d, 1H), 4.34 (bs, 2H), 4.53-4.82 (m, 1H), 5.30 (s, 1H), 7.22 (bs, 1H), 7.67 (bs, 1H), 8.04-8.18 (m, 1H), 8.39-8.56 (m, 1H), 9.95 (d, 1H)

### EXAMPLE 80

### 2-(6-Fluoroimidazo[1,2-a]pyridin-3-yl)-5-methyl-N-[(3R)-1-(3,3,3-trifluoropropanoyl)piperidin-3-yl]pyrimidin-4-amine

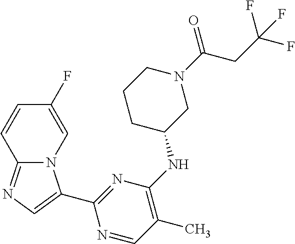 [SMILES: Cc1cnc(-c2cnc3ccc(F)cn23)nc1N[C@@H]1CCCN(C(=O)CC(F)(F)F)C1]

Obtained as a white solid (59%) from 2-(6-fluoroimidazo[1,2-a]pyridin-3-yl)-5-methyl-N-[(3R)-piperidin-3-yl]pyrimidin-4-amine (Preparation 20b) and 3,3,3-trifluoropropanoic acid following the experimental procedure as described in Example 54. The crude product was purified by flash chromatography (0-8% methanol in dichloromethane).

LRMS (m/z): 437 (M+1)⁺.

¹H-NMR δ (CDCl₃): 1.68 (s, 3H), 1.77 (bs, 1H), 1.86 (bs, 2H), 2.07 (d, 2H), 2.19 (bs, 1H), 3.12-3.23 (m, 1H), 3.62 (bs, 1H), 3.74-3.94 (m, 2H), 4.39 (bs, 1H), 4.99 (d, 1H), 7.16-7.26 (m, 1H), 7.62-7.73 (m, 1H), 8.11 (s, 1H), 8.41-8.54 (m, 1H), 8.48 (s, 1H), 9.96 (bs, 1H)

### EXAMPLE 81

### 3-((3R)-3-{[2-(6-Fluoroimidazo[1,2-a]pyridin-3-yl)pyrimidin-4-yl]amino}pyrrolidin-1-yl)-3-oxopropanenitrile

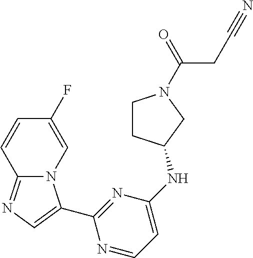 [SMILES: N#CCC(=O)N1CC[C@@H](Nc2ccnc(-c3cnc4ccc(F)cn34)n2)C1]

Obtained as a white solid (15%) from (R)-2-(6-fluoroimidazo[1,2-a]pyridin-3-yl)-N-(pyrrolidin-3-yl)pyrimidin-4-amine (Preparation 21b) and 3-[(2,5-dioxopyrrolidin-1-yl)oxy]-3-oxopropanenitrile (prepared as described in BE875054(A1)) following the experimental procedure as described in Example 46. The crude product was purified by flash chromatography (9:1 dichloromethane/methanol).

LRMS (m/z): 366 (M+1)⁺.

¹H-NMR δ (CDCl₃): 2.03-2.17 (m, 1H), 2.28-2.52 (m, 2H), 3.50 (s, 2H), 3.63-3.79 (m, 2H), 3.81-3.89 (m, 1H), 3.95-4.02 (m, 1H), 6.26 (dd, 1H), 7.20-7.31 (m, 1H), 7.69 (ddd, 1H), 8.28 (d, 1H), 8.51 (d, 1H), 9.98 (dd, 1H)

### EXAMPLE 82

### 3-{4-[4-(Cyanoacetyl)-1,4-diazepan-1-yl]pyrimidin-2-yl}imidazo[1,2-a]pyridine-6-carbonitrile

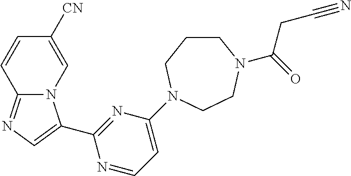 [SMILES: N#CCC(=O)N1CCCN(c2ccnc(-c3cnc4ccc(C#N)cn34)n2)CC1]

Obtained as a white solid (5%) from 3-(4-(1,4-diazepan-1-yl)pyrimidin-2-yl)imidazo[1,2-a]pyridine-6-carbonitrile (Preparation 22b) and 3-[(2,5-dioxopyrrolidin-1-yl)oxy]-3-oxopropanenitrile (prepared as described in BE875054(A1)) following the experimental procedure as described in Example 46. The crude product was purified by reverse phase chromatography (C-18 silica from Waters, water/1:1 acetonitrile-methanol as eluents [0.1% v/v formic acid buffered] 0% to 100%).

LRMS (m/z): 387 (M+1)⁺.

¹H-NMR δ (CDCl₃): 1.22-1.40 (m, 2H), 3.41 (s, 2H), 3.46-3.58 (m, 2H), 3.60 (bs, 2H), 3.65-3.94 (m, 2H), 4.26-4.38 (m, 2H), 6.41 (dd, 1H), 7.42 (ddd, 1H), 7.79 (dd, 1H), 8.35 (t, 1H), 8.55 (d, 1H), 10.55 (d, 1H)

### EXAMPLE 83

### 3-((3R)-3-{[5-Chloro-2-(6-fluoroimidazo[1,2-a]pyridin-3-yl)pyrimidin-4-yl]amino}piperidin-1-yl)-3-oxopropanenitrile

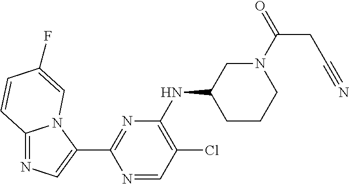

Obtained as a solid (68%) from 5-chloro-2-(6-fluoroimidazo[1,2-a]pyridin-3-yl)-N-[(3R)-piperidin-3-yl]pyrimidin-4-amine (Preparation 43b) and 3-[(2,5-dioxopyrrolidin-1-yl)oxy]-3-oxopropanenitrile (prepared as described in BE875054(A1)) following the experimental procedure as described in Example 46. The crude product was purified by flash chromatography (0-10% methanol in dichloromethane.

LRMS (m/z): 414 (M+1)⁺.

¹H NMR δ (CDCl₃): ppm 1.71-2.03 (m, 2H), 2.20 (dd, 1H), 3.29-3.52 (m, 2H), 3.57 (s, 2H), 3.59-3.70 (m, 1H), 3.80-3.94 (m, 1H), 4.23-4.42 (m, 1H), 5.33-5.49 (m, 1H), 7.29 (dd, 1H), 7.73 (dd, 1H), 8.27 (s, 1H), 8.56 (s, 1H), 9.82 (dd, 1H).

### EXAMPLE 84

### 3-((3R)-3-{[2-(6-Fluoroimidazo[1,2-a]pyridin-3-yl)pyrimidin-4-yl]amino}piperidin-1-yl)-2,2-dimethylpropanenitrile

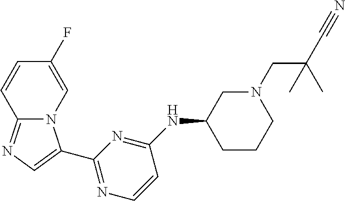 [SMILES: CC(C)(C#N)CN1CCC[C@@H](Nc2ccnc(-c3cnc4ccc(F)cn34)n2)C1]

(R)-2-(6-Fluoroimidazo[1,2-a]pyridin-3-yl)-N-(piperidin-3-yl)pyrimidin-4-amine (0.45 g, 1.44 mmol, Preparation 9b) and 2-cyano-2-methylpropyl 4-methylbenzenesulfonate (0.18 g, 0.72 mmol, Example 17b from US2007/299111A1) were mixed and stirred at 125° C. for 42 hours. The mixture was dissolved in ethyl acetate and washed with 1N sodium hydroxide and brine. The organic layer was dried and evaporated and the residue obtained was purified by reverse phase chromatography (C-18 silica from Waters, water/1:1 acetonitrile-methanol as eluents [0.1% v/v formic acid buffered] 0% to 100%) to give the title compound (3%) as a solid.

LRMS (m/z): 394 (M+1)⁺.

¹H NMR [symbol] (CDCl₃): 1.34 (s, 3H), 1.39 (s, 3H), 1.81 (bs, 4H), 2.39-2.53 (m, 2H), 2.60 (bs, 2H), 2.80 (br. s., 2H), 2.92 (bs, 1H), 5.67 (bs, 1H), 6.24 (d, 1H), 7.23 (ddd, 1H), 7.69 (dd, 1H), 8.21 (bs, 1H), 8.51 (s, 1H), 10.00 (dd, 1H).

### EXAMPLE 85

### 2-((3R)-3-{[2-(6-Fluoroimidazo[1,2-a]pyridin-3-yl)pyrimidin-4-yl]amino}piperidin-1-yl)acetamide

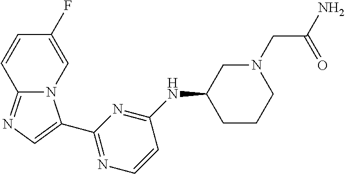 [SMILES: NC(=O)CN1CCC[C@@H](Nc2ccnc(-c3cnc4ccc(F)cn34)n2)C1]

Triethylamine (0.09 mL) followed by 2-bromoacetamide (0.049 g, 0.36 mmol) were added dropwise to a solution of (R)-2-(6-fluoroimidazo[1,2-a]pyridin-3-yl)-N-(piperidin-3-yl)pyrimidin-4-amine (0.1 g, 0.32 mmol, Preparation 9b) in dichloromethane (5 mL). The mixture was stirred at ambient temperature for 18 hours. The solvent was removed under reduced pressure and the residue was purified by reverse phase chromatography (C-18 silica from Waters, water/1:1 acetonitrile-methanol as eluents [0.1% v/v formic acid buffered] 0% to 100%) to obtain the title compound (61%) as a solid.

LRMS (m/z): 370 (M+1)⁺.

¹H NMR δ (DMSO-d₆): 1.46 (bs, 1H), 1.62 (bs, 1H), 1.71-1.89 (m, 2H), 2.36 (d, 2H), 2.83 (d, 1H), 3.17 (s, 2H), 4.28 (bs, 2H), 6.43 (d, 1H), 7.17 (bs, 1H), 7.37 (bs, 1H), 7.52 (ddd, 1H), 7.60 (d, 1H), 7.81 (dd, 1H), 8.29 (s, 1H), 8.40 (s, 1H), 9.97 (bs, 1H).

### EXAMPLE 86

### (S)-2-(6-Fluoroimidazo[1,2-a]pyridin-3-yl)-N-(1-(5-fluoropyridin-2-yl)ethyl)pyrimidin-4-amine

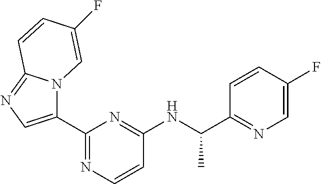 [SMILES: C[C@H](Nc1ccnc(-c2cnc3ccc(F)cn23)n1)c1ccc(F)cn1]

A mixture of 3-(4-chloropyrimidin-2-yl)-6-fluoroimidazo[1,2-a]pyridine (105 mg, 0.42 mmol, Preparation 44), (S)-1-(5-fluoropyridin-2-yl)ethanamine (66 mg, 0.47 mmol, prepared as described in WO2006/82392) and diisopropylethylamine (400 μL, 2.52 mmol) in THF (5 mL) was heated to reflux until completion of the reaction. The solvent was evaporated under reduced pressure and the residue was partitioned between dichloromethane and 4% aqueous sodium hydrogencarbonate solution. The organic layer was separated, washed with water and brine, dried over sodium sulphate and the solvent was removed under reduced pressure. The residue was purified by reverse phase chromatography (C-18 silica from Waters, water/1:1 acetonitrile-methanol as eluents [0.1% v/v formic acid buffered] 0% to 100%) to obtain the title compound (58 mg, 39%) as a solid.

LRMS (m/z): 353 (M+1)⁺.

¹H NMR [symbol] (CDCl₃): 1.66 (d, 3H), 5.15-5.44 (m, 1H), 5.98 (d, 1H), 6.24 (bs, 1H), 7.21-7.30 (m, 2H), 7.38-7.45 (m, 2H), 7.71 (dd, 1H), 8.25 (d, 1H), 8.42-8.56 (m, 1H), 9.94 (d, 1H).

### EXAMPLE 87

### 2-(6-Fluoroimidazo[1,2-a]pyridin-3-yl)-N-(pyridin-2-ylmethyl)pyrimidin-4-amine

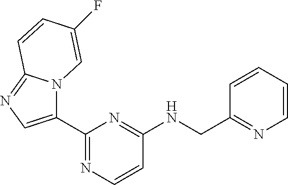 [SMILES: Fc1ccc2ncc(-c3nccc(NCc4ccccn4)n3)n2c1]

3-(4-Chloropyrimidin-2-yl)-6-fluoroimidazo[1,2-a]pyridine (43.7 mg, 0.18 mmol, Preparation 44), pyridin-2-yl-methanamine (36 μL, 0.35 mmol), 2′-(dicyclohexyl phosphino)-N,N-dimethylbiphenyl-2-amine (10 mg, 0.03 mmol), tris(dibenzylidene acetone) dipalladium (0) (13 mg, 0.01 mmol) and sodium tert-butoxide (24 mg, 0.25 mmol) in toluene (5 mL) were heated overnight to 110° C. under argon atmosphere. The solvent was removed under reduced pressure and the residue was purified by reverse phase chromatography (C-18 silica from Waters, water/1:1 acetonitrile-methanol as eluents [0.1% v/v formic acid buffered] 0% to 100%) to obtain the title compound (8 mg, 13%) as a solid.

LRMS (m/z): 321 (M+1)⁺.

¹H NMR [symbol] (CDCl₃): 4.80 (bs, 2H), 6.23 (bs, 1H), 6.32 (d, 1H), 7.14-7.29 (m, 3H), 7.40 (d, 1H), 7.70 (ddd, 1H), 8.26 (d, 1H), 8.54 (s, 1H), 8.61 (d, 1H), 9.96 (dd, 1H).

### EXAMPLE 88

### (S)-2-(6-Fluoroimidazo[1,2-a]pyridin-3-yl)-N-(1-(4-fluorophenyl)butyl)pyrimidin-4-amine

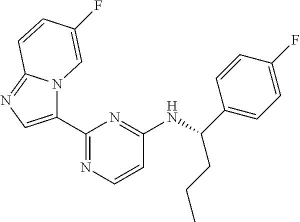 [SMILES: CCC[C@H](Nc1ccnc(-c2cnc3ccc(F)cn23)n1)c1ccc(F)cc1]

Obtained as a white solid (37%) from 3-(4-chloropyrimidin-2-yl)-6-fluoroimidazo[1,2-a]pyridine (50 mg, 0.2 mmol, Preparation 44) and (S)-1-(4-fluorophenyl)butan-1-amine (49 mg, 0.24 mmol, prepared as described in Tetrahedron:Asymmetry 2001, 12(15), 2185-2190) following the experimental procedure as described in Example 87.

LRMS (m/z): 380 (M+1)⁺.

¹H NMR [symbol] (CDCl₃): 0.98 (t, 3H), 1.31-1.53 (m, 2H), 1.72-2.04 (m, 2H), 4.82 (bs, 1H), 5.15-5.39 (m, 1H), 6.09 (bs, 1H), 7.04 (t, 2H), 7.22 (ddd, 1H), 7.34 (dd, 2H), 7.66 (dd, 1H), 8.20 (d, 1H), 8.49 (s, 1H), 9.86 (bs, 1H).

### EXAMPLE 89

### (R)-3-(2-(6-Fluoroimidazo[1,2-a]pyridin-3-yl)pyrimidin-4-ylamino)-1-propyl pyrrolidin-2-one

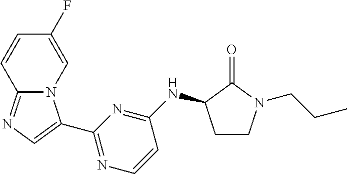 [SMILES: CCCN1CC[C@@H](Nc2ccnc(-c3cnc4ccc(F)cn34)n2)C1=O]

Obtained as a white solid (5%) from 2-(6-fluoroimidazo[1,2-a]pyridin-3-yl)pyrimidin-4-ol (122 mg, 0.53 mmol, Preparation 8b) and (R)-3-amino-1-propylpyrrolidin-2-one (170 mg, 0.63 mmol, prepared as described in J. Med. Chem. 1999, 42(18), 3557) following the experimental procedure as described in Preparation 5a.

LRMS (m/z): 355 (M+1)⁺.

¹H NMR [symbol] (CDCl₃): 0.95 (t, 3H), 1.77-1.37 (m, 2H), 2.18-1.77 (m, 2H), 3.67-3.21 (m, 4H), 5.82-5.75 (m, 1H), 6.33 (d, 1H), 7.37-7.16 (m, 1H), 7.74 (dd, 1H), 8.24 (t, 2H), 8.48 (d, 1H), 10.01 (d, 1H).

### EXAMPLE 90

### 6-Fluoro-3-(4-(2-(pyridin-2-yl)pyrrolidin-1-yl)pyrimidin-2-yl)imidazo[1,2-a]pyridine

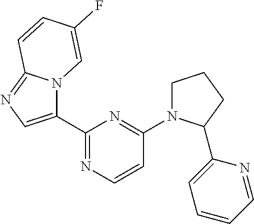 [SMILES: Fc1ccc2ncc(-c3nccc(N4CCCC4c4ccccn4)n3)n2c1]

Obtained as a white solid (22%) from 2-(6-fluoroimidazo[1,2-a]pyridin-3-yl)pyrimidin-4-ol (100 mg, 0.43 mmol, Preparation 8b) and 2-(pyrrolidin-2-yl)pyridine (129 mg, 0.87 mmol) following the experimental procedure as described in Preparation 5a.

LRMS (m/z): 361 (M+1)⁺.

¹H NMR [symbol] (CDCl₃): 1.99-2.36 (m, 6H), 2.42-2.73 (m, 2H), 3.45-3.90 (m, 2H), 3.92-4.29 (m, 2H), 4.99 (bs, 2H), 5.55 (bs, 1H), 5.89 (bs, 1H), 6.33 (bs, 1H), 7.05-7.23 (m, 4H), 7.51-7.76 (m, 4H), 8.01-8.40 (m, 4H), 8.52 (s, 1H), 8.63 (d, 2H), 9.34 (bs, 1H), 9.92-10.19 (m, 1H).

### EXAMPLE 91

### (S)—N,N-Diethyl-1-(2-(6-fluoroimidazo[1,2-a]pyridin-3-yl)pyrimidin-4-yl)pyrrolidine-2-carboxamide

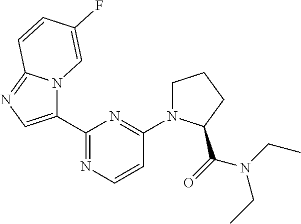 [SMILES: CCN(CC)C(=O)[C@@H]1CCCN1c1ccnc(-c2cnc3ccc(F)cn23)n1]

O-(7-Azabenzotriazol-1-yl)-N,N,N′,N′-tetramethyluronium hexafluorophosphate (46 mg, 0.12 mmol) and diisopropylethylamine (22 μL, 0.12 mmol) were added to a solution of (S)-1-(2-(6-fluoroimidazo[1,2-a]pyridin-3-yl)pyrimidin-4-yl)pyrrolidine-2-carboxylic acid (40 mg, 0.12 mmol, Preparation 45b) in N,N′-dimethylformamide (2 mL). After stirring for 30 min, diethylamine (16 μL, 0.14 mmol) was added and the reaction was stirred at room temperature for 3 additional hours. The solvent was evaporated and the residue was partitioned between water and chloroform. The organic layer was separated, washed with water and brine, dried over sodium sulphate and the solvent evaporated. The residue was purified by reverse phase chromatography (C-18 silica from Waters, water/1:1 acetonitrile-methanol as eluents [0.1% v/v formic acid buffered] 0% to 100%) to give the title compound (17 mg, 37%) as a solid.

LRMS (m/z): 382 (M⁺).

¹H NMR [symbol] (CDCl₃): 1.10 (t, 3H), 1.39 (t, 3H), 1.66 (bs, 2H), 2.09 (ddd, 2H), 2.27-2.41 (m, 2H), 3.30-3.64 (m, 2H), 3.65-3.84 (m, 2H), 5.10 (bs, 1H), 6.29 (d, 1H), 7.20 (ddd, 1H), 7.63 (dd, 1H), 8.27 (d, 1H), 8.34 (bs, 1H), 9.93 (bs, 1H).

### EXAMPLE 92

### (R)—N,N-Diethyl-1-(2-(6-fluoroimidazo[1,2-a]pyridin-3-yl)pyrimidin-4-yl)pyrrolidine-2-carboxamide

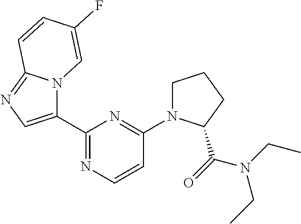 [SMILES: CCN(CC)C(=O)[C@H]1CCCN1c1ccnc(-c2cnc3ccc(F)cn23)n1]

Obtained as a white solid (22%) from (R)-1-(2-(6-fluoroimidazo[1,2-a]pyridin-3-yl)pyrimidin-4-yl)pyrrolidine-2-carboxylic acid (40 mg, 0.12 mmol, Preparation 46b) and diethylamine (15 μL, 0.14 mmol) following the experimental procedure as described in Example 91.

LRMS (m/z): 382 (M⁺).

¹H NMR [symbol] (CDCl₃): 1.10 (t, 3H), 1.39 (t, 3H), 1.99-2.16 (m, 2H), 2.26-2.41 (m, 2H), 3.29-3.63 (m, 5H), 3.67-3.85 (m, 1H), 5.09 (bs, 1H), 6.28 (bs, 1H), 7.20 (dd, 1H), 7.63 (dd, 1H), 8.28 (d, 1H), 8.35 (bs, 1H), 9.93 (bs, 1H).

### EXAMPLE 93

### 2-((1r,4r)-4-(2-(6-Fluoroimidazo[1,2-a]pyridin-3-yl)pyrimidin-4-ylamino)cyclohexyl)acetonitrile

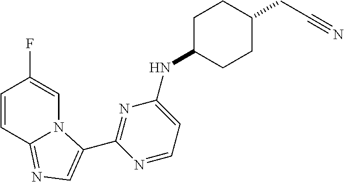

Obtained as a white-off solid (21%) from 3-(4-chloropyrimidin-2-yl)-6-fluoroimidazo[1,2-a]pyridine (90 mg, 0.36 mmol, Preparation 44) and 2-((1r,4r)-4-aminocyclohexyl)acetonitrile (50 mg, 0.36 mmol, Preparation 47b) following the experimental procedure as described in Example 86.

LRMS (m/z): 351 (M+1)⁺.

¹H-NMR δ (CDCl₃): 0.8 (m, 4H), 1.22-1.38 (m, 4H), 1.8 (m, 1H), 2.0 (m, 1H), 2.3 (d, 2H), 4.9 (bs, 1H), 6.2 (d, 1H), 7.2 (m, 1H), 7.7 (dd, 1H), 8.2 (m, 1H), 8.5 (s, 1H), 10.0 (dd, 1H).

### EXAMPLE 94

### (R)-2-(3-(2-(6-Fluoroimidazo[1,2-a]pyridin-3-yl)pyrimidin-4-ylamino)piperidin-1-ylsulfonyl)acetonitrile

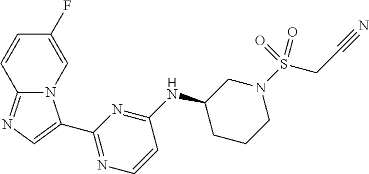 [SMILES: N#CCS(=O)(=O)N1CCC[C@@H](Nc2ccnc(-c3cnc4ccc(F)cn34)n2)C1]

A solution of (R)-2-(6-fluoroimidazo[1,2-a]pyridin-3-yl)-N-(piperidin-3-yl)pyrimidin-4-amine (130 mg, 0.42 mmol, Preparation 9b), cyanomethanesulfonyl chloride (58 mg, 0.42 mmol, prepared as described in GB125903) and triethylamine (58 μL, 0.42 mmol) in dichloromethane (2 mL) was stirred at room temperature for 48 hours. The reaction mixture was diluted with water and the organic phase was separated, washed with 4% aqueous sodium hydrogencarbonate solution, dried over sodium sulphate, filtered and the solvent evaporated under reduced pressure. The residue was purified by reverse phase chromatography (C-18 silica from Waters, water/1:1 acetonitrile-methanol as eluents [0.1% v/v formic acid buffered] 0% to 100%) to obtain the title compound (5 mg, 3%).

LRMS (m/z): 416 (M+1)⁺.

¹H NMR [symbol] (CD₃OD): 1.42-2.16 (m, 6H), 2.86 (m, 2H), 3.67 (m, 2H), 3.88-4.34 (m, 1H), 6.36 (bs, 1H), 7.32-7.54 (m, 1H), 7.58-7.77 (m, 2H), 7.95-8.23 (m, 1H), 8.47 (bs, 1H), 9.97 (bs, 1H).

### EXAMPLE 95

### (R)-4,4,4-Trifluoro-1-(3-(2-(6-fluoroimidazo[1,2-a]pyridin-3-yl)pyrimidin-4-ylamino) piperidin-1-yl)butan-1-one

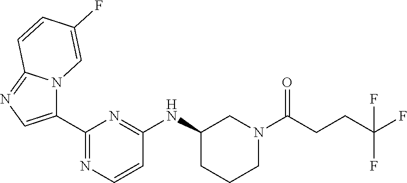 [SMILES: O=C(CCC(F)(F)F)N1CCC[C@@H](Nc2ccnc(-c3cnc4ccc(F)cn34)n2)C1]

N-Methylmorpholine (107 mg, 0.96 mmol), N-(3-dimethylaminopropyl)-W-ethyl carbodiimide hydrochloride (92 mg, 0.48 mmol), 1-hydroxybenzotriazole (65 mg, 0.48 mmol) and (R)-2-(6-fluoroimidazo[1,2-a]pyridin-3-yl)-N-(piperidin-3-yl)pyrimidin-4-amine (30 mg, 0.1 mmol, Preparation 9b) were added to a solution of 4,4,4-trifluorobutanoic acid (27 mg, 0.19 mmol) in N,N-dimethylacetamide (1 mL). After the mixture was stirred overnight the solvent was removed under vacuum in a DD-4 apparatus. The residue was dissolved in ethyl acetate and the organic solution was washed with water, saturated aqueous hydrogencarbonate solution and brine, dried over sodium sulphate and the solvent evaporated. The crude product was purified by semi preparative HPLC in basic media (water with ammonium hydrogencarbonate 10 mM and acetonitrile) to yield the final compound (33.8 mg, 80%) as a white solid.

LRMS (m/z): 437 (M+1)⁺.

¹H NMR [symbol] (CDCl₃): 1.83 (bs, 2H), 2.34-2.74 (m, 6H), 3.18-3.43 (m, 2H), 3.54-4.40 (m, 3H), 5.17 (bs, 1H), 6.21 (d, 1H), 7.21 (t, 1H), 7.68 (dd, 1H), 8.24 (d, 1H), 8.51 (bs, 1H), 9.94 (d, 1H).

### EXAMPLE 96

### ((R)-3-(2-(6-Fluoroimidazo[1,2-a]pyridin-3-yl)pyrimidin-4-ylamino)piperidin-1-yl)((1S,2R)-2-phenylcyclopropyl)methanone

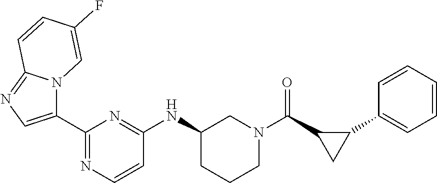

Obtained as a white solid (20 mg, 45%) from (1S,2R)-2-phenylcyclopropanecarboxylic acid (31 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 457 (M+1)⁺.

¹H NMR [symbol] (CD₃OD): 0.96-1.08 (m, 1H), 1.25-1.36 (m, 1H), 1.53 (d, 1H), 1.64-1.74 (m, 2H), 1.75-1.84 (m, 1H), 1.90 (bs, 1H), 2.02-2.08 (m, 1H), 2.11-2.28 (m, 2H), 2.30-2.48 (m, 1H), 3.38-3.90 (m, 1H), 4.01-4.24 (m, 2H), 6.30 (d, 1H), 6.59 (bs, 2H), 6.72-6.81 (m, 2H), 7.20 (m, 1H), 7.28 (m, 1H), 7.42-7.55 (m, 1H), 7.64-7.79 (m, 1H), 7.93-8.23 (m, 1H), 8.36 (s, 1H).

### EXAMPLE 97

### (R)-1-((R)-3-(2-(6-Fluoroimidazo[1,2-a]pyridin-3-yl)pyrimidin-4-ylamino)piperidin-1-yl)-2-hydroxy-3,3-dimethylbutan-1-one

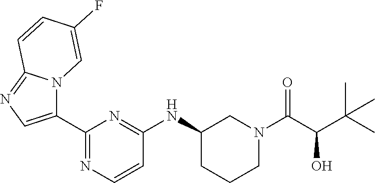 [SMILES: CC(C)(C)[C@@H](O)C(=O)N1CCC[C@@H](Nc2ccnc(-c3cnc4ccc(F)cn34)n2)C1]

Obtained as a white solid (32 mg, 78%) from (R)-2-hydroxy-3,3-dimethylbutanoic acid (25 mg, 0.19 mmol) and (R)-2-(6-fluoroimidazo[1,2-a]pyridin-3-yl)-N-(piperidin-3-yl) pyrimidin-4-amine (30 mg, 0.1 mmol, Preparation 9b) following the experimental procedure as described in Example 95.

LRMS (m/z): 427 (M+1)⁺.

¹H NMR [symbol] (CD₃OD): 0.98 (s, 9H), 1.57-1.97 (m, 4H), 2.09-2.30 (m, 1H), 2.69-2.80 (m, 1H), 3.20 (d, 1H), 4.01-4.12 (m, 1H), 4.34 (s, 1H), 4.84 (s, 2H), 6.43 (d, 1H), 7.37-7.52 (m, 1H), 7.68 (dd, 1H), 8.09-8.22 (m, 1H), 8.41 (s, 1H), 8.51-8.59 (m, 1H), 10.02 (bs, 1H).

### EXAMPLE 98

### (R)-2-Cyclopentyl-1-(342-(6-fluoroimidazo[1,2-a]pyridin-3-yl)pyrimidin-4-ylamino) piperidin-1-yl)ethanone

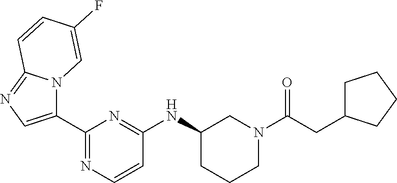 [SMILES: O=C(CC1CCCC1)N1CCC[C@@H](Nc2ccnc(-c3cnc4ccc(F)cn34)n2)C1]

Obtained as a white solid (29 mg, 70%) from 2-cyclopentylacetic acid (24 μL, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 423 (M+1)⁺.

¹H NMR [symbol] (CD₃OD): 0.94 (bs, 1H), 1.14-1.28 (m, 1H), 1.32-1.49 (m, 2H), 1.50-1.75 (m, 6H), 1.78-1.99 (m, 3H), 2.04-2.33 (m, 2H), 2.46 (d, 1H), 2.84 (dd, 1H), 3.26 (dd, 1H), 3.42 (dd, 1H), 3.91 (d, 1H), 6.42 (d, 1H), 7.45 (ddd, 1H), 7.70 (td, 1H), 8.18 (d, 1H), 8.41 (s, 1H), 8.46 (s, 1H), 10.02 (d, 1H).

### EXAMPLE 99

### (R)-1-(2-(3-(2-(6-Fluoroimidazo[1,2-a]pyridin-3-yl)pyrimidin-4-ylamino)piperidin-1-yl)-2-oxoethyl)-5-methylpyrimidine-2,4(1H,3H)-dione

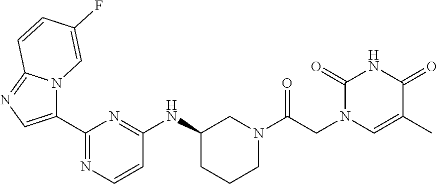 [SMILES: Cc1cn(CC(=O)N2CCC[C@@H](Nc3ccnc(-c4cnc5ccc(F)cn45)n3)C2)c(=O)[nH]c1=O]

Obtained as a white solid (21 mg, 45%) from 2-(5-methyl-2,4-dioxo-3,4-dihydro pyrimidin-1(2H)-yl)acetic acid (35 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 479 (M+1)⁺.

¹H NMR [symbol] (CD₃OD): 1.52-1.83 (m, 4H), 1.87 (d, 3H), 1.94-2.03 (m, 2H), 2.08-2.26 (m, 1H), 2.91-3.07 (m, 1H), 3.35-3.48 (m, 2H), 3.68-4.02 (m, 2H), 4.42-4.77 (m, 1H), 6.31-6.54 (m, 1H), 7.18-7.33 (m, 1H), 7.37-7.51 (m, 1H), 7.68 (dt, 1H), 8.07-8.26 (m, 1H), 8.46 (d, 1H), 10.05 (bs, 1H).

### EXAMPLE 100

### (R)-1-(3-(2-(6-Fluoroimidazo[1,2-a]pyridin-3-yl)pyrimidin-4-ylamino)piperidin-1-yl)-2-(1H-1,2,4-triazol-1-yl)ethanone

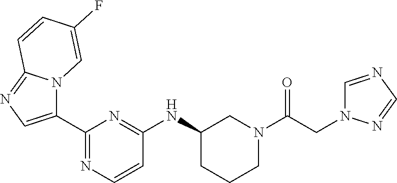 [SMILES: O=C(Cn1cncn1)N1CCC[C@@H](Nc2ccnc(-c3cnc4ccc(F)cn34)n2)C1]

Obtained as a white solid (10 mg, 25%) from 2-(1H-1,2,4-triazol-1-yl)acetic acid (24 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 422 (M+1)⁺.

¹H NMR [symbol] (CD₃OD): 1.76 (ddd, 3H), 1.84-2.03 (m, 2H), 2.18 (dd, 1H), 2.94 (dd, 1H), 3.80-4.06 (m, 2H), 4.56 (bs, 1H), 5.35 (d, 1H), 6.37 (d, 1H), 6.47 (d, 1H), 7.34-7.52 (m, 1H), 7.67 (ddd, 1H), 7.94-8.03 (m, 1H), 8.09-8.25 (m, 1H), 8.44 (m, 2H), 10.04 (bs, 1H).

### EXAMPLE 101

### (R)-(3-(2-(6-Fluoroimidazo[1,2-a]pyridin-3-yl)pyrimidin-4-ylamino)piperidin-1-yl)(1-methylcyclohexyl)methanone

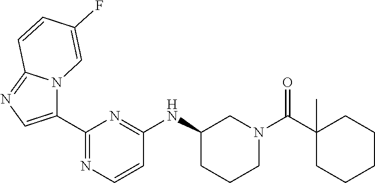 [SMILES: CC1(C(=O)N2CCC[C@@H](Nc3ccnc(-c4cnc5ccc(F)cn45)n3)C2)CCCCC1]

Obtained as a white solid (13 mg, 31%) from 1-methylcyclohexanecarboxylic acid (27 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 437 (M+1)⁺.

¹H NMR [symbol] (CD₃OD): 1.25 (s, 3H), 1.30-1.57 (m, 9H), 1.58-1.71 (m, 2H), 1.83-1.95 (m, 1H), 2.04 (d, 2H), 2.20 (s, 1H), 2.80-2.95 (m, 1H), 3.03-3.18 (m, 1H), 4.13 (b s, 1H), 4.23 (d, 1H), 4.52 (d, 1H), 6.38 (bs, 1H), 7.45 (ddd, 1H), 7.69 (dd, 1H), 8.15 (bs, 1H), 8.43 (s, 1H), 10.02 (bs, 1H).

### EXAMPLE 102

### (2,2-Difluorocyclopropyl)((R)-3-(2-(6-fluoroimidazo[1,2-a]pyridin-3-yl)pyrimidin-4-ylamino)piperidin-1-yl)methanone

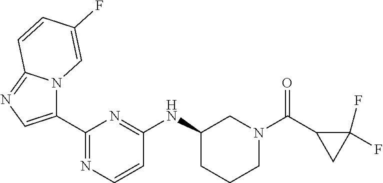 [SMILES: O=C(C1CC1(F)F)N1CCC[C@@H](Nc2ccnc(-c3cnc4ccc(F)cn34)n2)C1]

Obtained as a white solid (32 mg, 79%) from 2,2-difluorocyclopropanecarboxylic acid (23 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 417 (M+1)⁺.

¹H NMR [symbol] (CD₃OD): 1.48-1.85 (m, 4H), 1.85-2.06 (m, 3H), 2.10-2.28 (m, 2H), 2.79-3.09 (m, 1H), 3.93-4.10 (m, 2H), 6.31-6.49 (m, 1H), 7.46 (m, 1H), 7.70 (m, 1H), 8.19 (m, 1H), 8.41 (d, 1H), 8.45-8.52 (m, 1H), 10.04 (bs, 1H).

### EXAMPLE 103

### ((R)-3-(2-(6-Fluoroimidazo[1,2-a]pyridin-3-yl)pyrimidin-4-ylamino)piperidin-1-yl)((1R,2S)-2-hydroxycyclopentyl)methanone

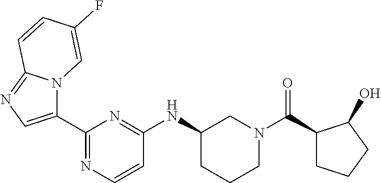 [SMILES: O=C([C@@H]1CCC[C@@H]1O)N1CCC[C@@H](Nc2ccnc(-c3cnc4ccc(F)cn34)n2)C1]

Obtained as a white solid (27 mg, 67%) from (1R,2S)-2-hydroxycyclopentanecarboxylic acid (25 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 425 (M+1)⁺.

¹H NMR [symbol] (CD₃OD): 1.50-1.99 (m, 8H), 2.15 (s, 2H), 2.79-3.04 (m, 1H), 3.06-3.15 (m, 1H), 3.41-3.53 (m, 1H), 3.75-3.86 (m, 1H), 3.89-4.29 (m, 1H), 4.31-4.49 (m, 1H), 4.65 (d, 1H), 4.59-4.69 (m, 1H), 6.42 (d, 1H), 7.34-7.57 (m, 1H), 7.61-7.79 (m, 1H), 8.04-8.26 (m, 1H), 8.32-8.55 (m, 2H), 10.02 (bs, 1H).

### EXAMPLE 104

### (R)-1-(3-(2-(6-Fluoroimidazo[1,2-a]pyridin-3-yl)pyrimidin-4-ylamino)piperidin-1-yl)-2-(1H-pyrazol-1-yl)ethanone

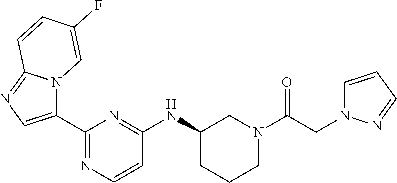 [SMILES: O=C(Cn1cccn1)N1CCC[C@@H](Nc2ccnc(-c3cnc4ccc(F)cn34)n2)C1]

Obtained as a white solid (25.5 mg, 63%) from 2-(1H-pyrazol-1-yl)acetic acid (24 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 421 (M+1)⁺.

¹H NMR [symbol] (CD₃OD): 1.59-1.97 (m, 4H), 2.18 (bs, 2H), 3.02 (t, 1H), 3.84 (d, 1H), 3.98 (d, 1H), 4.48 (d, 1H), 4.97-5.17 (m, 2H), 6.33 (t, 1H), 6.35-6.54 (m, 1H), 7.35-7.60 (m, 2H), 7.62-7.74 (m, 2H), 8.07-8.26 (m, 1H), 8.38-8.50 (m, 1H), 10.05 (bs, 1H).

### EXAMPLE 105

### (R)-Cyclohexyl(3-(2-(6-Fluoroimidazo[1,2-a]pyridin-3-yl)pyrimidin-4-ylamino) piperidin-1-yl)methanone

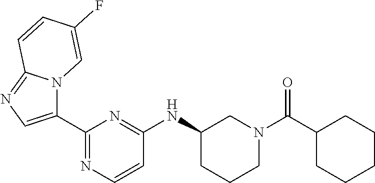 [SMILES: O=C(C1CCCCC1)N1CCC[C@@H](Nc2ccnc(-c3cnc4ccc(F)cn34)n2)C1]

Obtained as a white solid (28 mg, 67%) from cyclohexanoic acid (25 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 423 (M+1)⁺.

¹H NMR [symbol] (CD₃OD): 1.23-2.01 (m, 7H), 2.07-2.25 (m, 2H), 2.40 (t, 1H), 2.63-2.73 (m, 1H), 2.78 (t, 1H), 3.21 (m, 1H), 3.40 (m, 1H), 3.56-3.66 (m, 1H), 3.79 (d, 1H), 3.83-3.99 (m, 2H), 4.15 (bs, 1H), 4.65 (d, 1H), 6.43 (d, 1H), 7.47 (t, 1H), 7.63-7.76 (m, 2H), 8.09-8.26 (m, 1H), 8.37-8.51 (m, 1H), 10.02 (bs, 1H).

### EXAMPLE 106

### (R)-(3-(2-(6-Fluoroimidazo[1,2-a]pyridin-3-yl)pyrimidin-4-ylamino)piperidin-1-yl)(4-methyl-1,2,3-thiadiazol-5-yl)methanone

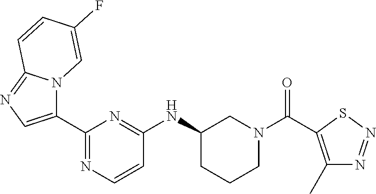 [SMILES: Cc1nnsc1C(=O)N1CCC[C@@H](Nc2ccnc(-c3cnc4ccc(F)cn34)n2)C1]

Obtained as a white solid (26.8 mg, 63%) from 4-methyl-1,2,3-thiadiazole-5-carboxylic acid (28 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 439 (M+1)⁺.

¹H NMR [symbol] (CD₃OD): 1.57-2.27 (m, 8H), 2.51 (s, 3H), 2.73 (s, 3H), 3.13 (m, 2H), 3.25 (m, 2H), 3.36-3.62 (m, 2H), 4.03 (m, 2H), 4.18 (m, 2H), 6.41 (bs, 2H), 7.37-7.51 (m, 2H), 7.69 (dd, 2H), 8.16 (bs, 5H), 8.52 (bs, 1H), 9.85 (bs, 1H), 10.04 (bs, 1H).

### EXAMPLE 107

### (R)-1-(3-(2-(6-Fluoroimidazo[1,2-a]pyridin-3-yl)pyrimidin-4-ylamino)piperidin-1-yl)-2-(4-(hydroxymethyl)phenyl)ethanone

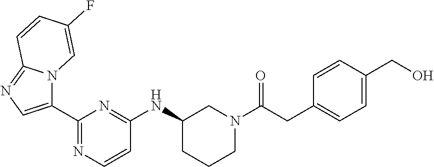 [SMILES: O=C(Cc1ccc(CO)cc1)N1CCC[C@@H](Nc2ccnc(-c3cnc4ccc(F)cn34)n2)C1]

Obtained as a white solid (14.5 mg, 33%) from 2-(4-(hydroxymethyl)phenyl)acetic acid (32 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 461 (M+1)⁺.

¹H NMR [symbol] (CD₃OD): 1.46 (m, 1H), 1.65 (m, 2H), 1.87 (m, 1H), 2.10 (m, 1H), 2.86 (dd, 1H), 3.17 (t, 1H), 3.83 (s, 2H), 3.83-3.91 (m, 1H), 4.33 (bs, 1H), 4.59 (s, 2H), 4.63 (d, 1H), 6.37 (bs, 1H), 7.02 (bs, 2H), 7.24-7.31 (m, 2H), 7.31-7.38 (m, 2H), 7.46 (dd, 1H), 7.70 (td, 1H), 8.14 (d, 1H), 8.36-8.51 (m, 2H), 9.93 (bs, 1H), 10.03 (bs, 1H).

### EXAMPLE 108

### (S)-1-((R)-3-(2-(6-Fluoroimidazo[1,2-a]pyridin-3-yl)pyrimidin-4-ylamino)piperidin-1-yl)-3-phenylbutan-1-one

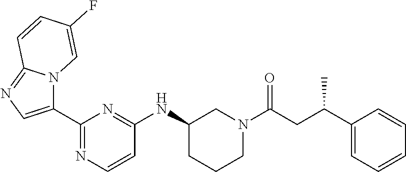 [SMILES: C[C@@H](CC(=O)N1CCC[C@@H](Nc2ccnc(-c3cnc4ccc(F)cn34)n2)C1)c1ccccc1]

Obtained as a white solid (9 mg, 5%) from (S)-3-phenylbutanoic acid (29 μL, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 459 (M+1)⁺.

¹H NMR [symbol] (CD₃OD): 1.11 (d, 3H), 1.34 (d, 3H), 1.48-1.96 (m, 8H), 2.06-2.23 (m, 4H), 2.25-2.43 (m, 1H), 2.47-2.57 (m, 1H), 2.60-2.68 (m, 1H), 2.71-2.85 (m, 2H), 2.98 (d, 1H), 3.05-3.15 (m, 1H), 3.19-3.28 (m, 1H), 3.65-3.76 (m, 2H), 3.79 (d, 1H), 4.56 (bs, 1H), 6.35 (d, 1H), 6.42 (d, 2H), 7.17 (td, 1H), 7.24-7.29 (m, 6H), 7.45 (qd, 2H), 7.66 (dd, 2H), 8.14 (bs, 1H), 8.18 (d, 2H), 8.38 (s, 2H), 8.46 (s, 1H), 9.94 (bs, 2H), 10.03 (bs, 1H).

### EXAMPLE 109

### (R)-1-((R)-3-(2-(6-Fluoroimidazo[1,2-a]pyridin-3-yl)pyrimidin-4-ylamino)piperidin-1-yl)-3-phenylbutan-1-one

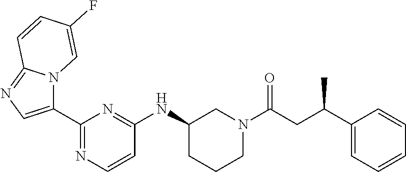 [SMILES: C[C@H](CC(=O)N1CCC[C@@H](Nc2ccnc(-c3cnc4ccc(F)cn34)n2)C1)c1ccccc1]

Obtained as a white solid (10 mg, 23%) from (R)-3-phenylbutanoic acid (32 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 459 (M+1)⁺.

¹H NMR [symbol] (CD₃OD): 1.11 (d, 3H), 1.34 (d, 3H), 1.52-1.78 (m, 8H), 1.81-1.92 (m, 1H), 1.99-2.09 (m, 1H), 2.09-2.16 (m, 1H), 2.36-2.47 (m, 1H), 2.60 (ddd, 2H), 2.74 (d, 1H), 2.76-2.84 (m, 1H), 2.98-3.08 (m, 1H), 3.09-3.25 (m, 3H), 3.77 (d, 1H), 3.87-4.00 (m, 3H), 4.59 (d, 1H), 6.34 (d, 1H), 6.39 (d, 1H), 6.79-6.88 (m, 2H), 6.88-6.96 (m, 4H), 7.19 (t, 1H), 7.26-7.34 (m, 4H), 7.41-7.51 (m, 2H), 7.70 (dd, 2H), 8.16 (d, 2H), 8.38 (s, 1H), 8.49 (s, 1H), 9.94-10.00 (m, 1H), 10.01-10.07 (m, 1H).

### EXAMPLE 110

### (R)-1-(3-(2-(6-Fluoroimidazo[1,2-a]pyridin-3-yl)pyrimidin-4-ylamino)piperidin-1-yl)-3-(4-methoxyphenyl)propan-1-one

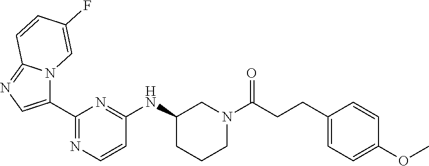 [SMILES: COc1ccc(CCC(=O)N2CCC[C@@H](Nc3ccnc(-c4cnc5ccc(F)cn45)n3)C2)cc1]

Obtained as a white solid (22 mg, 47%) from 3-(4-methoxyphenyl)propanoic acid (35 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 475 (M+1)⁺.

¹H NMR [symbol] (CD₃OD): 1.37-1.97 (m, 8H), 2.06-2.20 (m, 2H), 2.43-2.59 (m, 3H), 2.66-2.84 (m, 4H), 2.86-2.95 (m, 2H), 3.06-3.17 (m, 1H), 3.41 (bs, 1H), 3.54 (s, 3H), 3.75 (s, 3H), 3.77-3.92 (m, 4H), 4.65 (d, 1H), 6.39 (d, 6H), 6.76 (d, 1H), 6.79-6.87 (m, 4H), 7.19 (t, 1H), 7.37-7.52 (m, 2H), 7.66 (td, 2H), 8.15 (d, 2H), 8.35 (s, 1H), 8.50 (s, 1H), 9.90-9.99 (m, 1H), 9.99-10.07 (m, 1H).

### EXAMPLE 111

### (R)-(5-Fluoro-2,6-dihydroxypyrimidin-4-yl)(3-(2-(6-fluoroimidazo[1,2-a]pyridin-3-yl)pyrimidin-4-ylamino)piperidin-1-yl)methanone

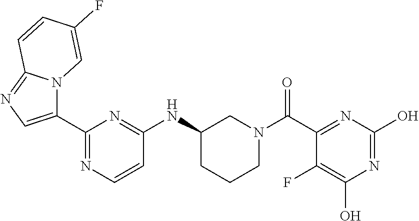 [SMILES: O=C(c1nc(O)nc(O)c1F)N1CCC[C@@H](Nc2ccnc(-c3cnc4ccc(F)cn34)n2)C1]

Obtained as a white solid (37 mg, 82%) from 5-fluoro-2,6-dihydroxypyrimidine-4-carboxylic acid (33 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 469 (M+1)⁺.

¹H NMR [symbol] (CD₃OD): 1.69-1.82 (m, 1H), 1.88-2.07 (m, 3H), 2.11-2.28 (m, 1H), 2.98 (m, 1H), 3.66 (m, 1H), 3.79 (m, 1H), 4.10 (m, 1H), 6.43 (d, 1H), 7.42-7.54 (m, 1H), 7.71 (dd, 1H), 8.17 (d, 1H), 8.22 (d, 1H), 8.35 (s, 1H), 8.58 (bs, 1H), 10.02 (bs, 1H), 10.07 (bs, 1H).

### EXAMPLE 112

### (R)-2-(3-Chlorophenyl)-1-((R)-3-(2-(6-fluoroimidazo[1,2-a]pyridin-3-yl)pyrimidin-4-ylamino)piperidin-1-yl)-2-hydroxyethanone

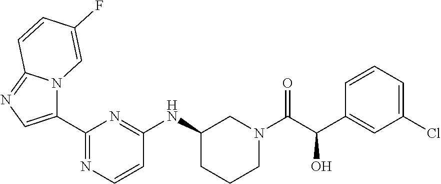 [SMILES: O=C([C@H](O)c1cccc(Cl)c1)N1CCC[C@@H](Nc2ccnc(-c3cnc4ccc(F)cn34)n2)C1]

Obtained as a white solid (9 mg, 20%) from (R)-2-(3-chlorophenyl)-2-hydroxyacetic acid (36 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 481 (M+1)⁺.

¹H NMR [symbol] (CD₃OD): 0.93-1.11 (m, 2H), 1.62 (m, 6H), 1.98-2.20 (m, 4H), 2.79-2.95 (m, 2H), 3.02-3.22 (m, 4H), 3.70-3.83 (m, 2H), 3.98-4.17 (m, 3H), 4.64-4.76 (m, 1H), 5.40 (s, 1H), 5.47 (s, 1H), 6.38 (bs, 1H), 6.85-7.25 (m, 2H), 7.34-7.42 (m, 4H), 7.42-7.54 (m, 3H), 7.65-7.78 (m, 2H), 8.01-8.22 (m, 2H), 8.46 (s, 1H), 8.52 (s, 1H), 9.92 (bs, 1H), 10.02 (bs, 1H).

### EXAMPLE 113

### (R)-(3-(2-(6-Fluoroimidazo[1,2-a]pyridin-3-yl)pyrimidin-4-ylamino)piperidin-1-yl)(pyrimidin-5-yl)methanone

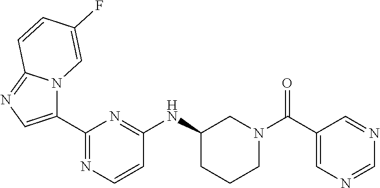 [SMILES: O=C(c1cncnc1)N1CCC[C@@H](Nc2ccnc(-c3cnc4ccc(F)cn34)n2)C1]

Obtained as a white solid (8 mg, 19%) from pyrimidine-5-carboxylic acid (24 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 419 (M+1)⁺.

¹H NMR [symbol] (CD₃OD): 1.68-1.81 (m, 3H) 1.83-1.97 (m, 2H) 2.00-2.27 (m, 3H) 3.02 (d, 2H) 3.48 (m, 2H) 3.66 (d, 2H) 3.93-4.25 (m, 4H) 6.41 (bs, 2H) 7.46 (t, 2H) 7.69 (bs, 2H) 7.95-8.25 (m, 4H) 8.52 (bs, 1H) 8.61 (bs, 4H) 8.91 (s, 2H) 9.26 (s, 1H) 9.84 (bs, 1H) 10.06 (bs, 1H)

### EXAMPLE 114

### (R)-(3-(2-(6-Fluoroimidazo[1,2-a]pyridin-3-yl)pyrimidin-4-ylamino)piperidin-1-yl)(1-(trifluoromethyl)cyclobutyl)methanone

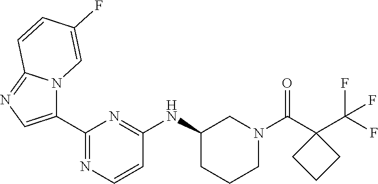 [SMILES: O=C(N1CCC[C@@H](Nc2ccnc(-c3cnc4ccc(F)cn34)n2)C1)C1(C(F)(F)F)CCC1]

Obtained as a white solid (23 mg, 51%) from 1-(trifluoromethyl)cyclobutanecarboxylic acid (32 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 463 (M+1)⁺.

¹H NMR [symbol] (CD₃OD): 1.60-1.80 (m, 3H), 1.90 (m, 3H), 2.01-2.16 (m, 1H), 2.17-2.25 (m, 1H), 2.31-2.44 (m, 1H), 2.45-2.62 (m, 2H), 2.67-2.96 (m, 2H), 3.08-3.21 (m, 1H), 3.55-3.79 (m, 1H), 6.39 (d, 1H), 7.46 (ddd, 1H), 7.70 (dd, 1H), 8.17 (bs, 1H), 8.40 (bs, 1H), 8.49 (bs, 1H), 9.91-10.13 (m, 1H).

### EXAMPLE 115

### (R)-(3-(2-(6-Fluoroimidazo[1,2-a]pyridin-3-yl)pyrimidin-4-ylamino)piperidin-1-yl)(1-hydroxycyclopropyl)methanone

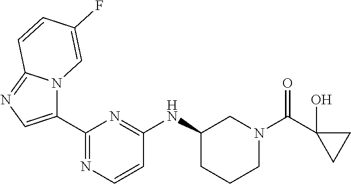 [SMILES: O=C(N1CCC[C@@H](Nc2ccnc(-c3cnc4ccc(F)cn34)n2)C1)C1(O)CC1]

Obtained as a white solid (25 mg, 65%) from 1-hydroxycyclopropanecarboxylic acid (20 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 497 (M+1)⁺.

¹H NMR [symbol] (CD₃OD): 0.68-1.17 (m, 8H), 1.58-1.82 (m, 4H), 1.84-1.98 (m, 1H), 2.14-2.28 (m, 1H), 4.52 (bs, 1H), 6.38 (bs, 1H), 7.44 (ddd, 1H), 7.68 (dd, 1H), 8.15 (bs, 1H), 8.45 (s, 1H), 10.02 (bs, 1H).

### EXAMPLE 116

### (R)-2-(Benzyloxy)-1-(3-(2-(6-fluoroimidazo[1,2-a]pyridin-3-yl)pyrimidin-4-ylamino) piperidin-1-yl)ethanone

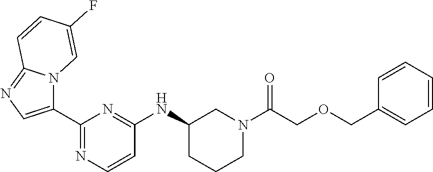 [SMILES: O=C(COCc1ccccc1)N1CCC[C@@H](Nc2ccnc(-c3cnc4ccc(F)cn34)n2)C1]

Obtained as a white solid (40 mg, 90%) from 2-(benzyloxy)acetic acid (28 μL, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 461 (M+1)⁺.

¹H NMR [symbol] (CD₃OD): 1.55-1.79 (m, 5H), 1.81-1.95 (m, 1H), 2.09-2.22 (m, 3H), 2.89 (dd, 1H), 3.18 (t, 1H), 3.79 (d, 1H), 3.90 (d, 3H), 4.00-4.21 (m, 4H), 4.27 (d, 2H), 4.38 (bs, 2H), 4.51-4.59 (m, 1H), 4.60 (s, 2H), 6.34 (d, 1H), 6.38 (d, 1H), 6.99 (bs, 3H), 7.08 (bs, 3H), 7.23-7.49 (m, 8H), 7.69 (ddd, 2H), 8.12 (d, 2H), 8.40 (s, 1H), 8.47 (s, 1H), 9.90 (bs, 1H), 10.02 (bs, 1H).

### EXAMPLE 117

### (R)-(3-(2-(6-Fluoroimidazo[1,2-a]pyridin-3-yl)pyrimidin-4-ylamino)piperidin-1-yl)(1H-indol-2-yl)methanone

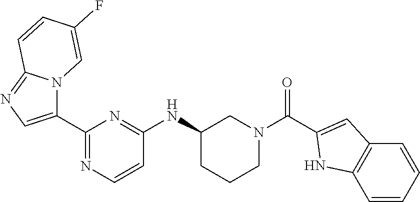 [SMILES: O=C(c1cc2ccccc2[nH]1)N1CCC[C@@H](Nc2ccnc(-c3cnc4ccc(F)cn34)n2)C1]

Obtained as a white solid (34 mg, 77%) from 1H-indole-2-carboxylic acid (31 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 456 (M+1)⁺.

¹H NMR [symbol] (CD₃OD): 1.79 (m, 4H), 2.04 (m, 2H), 2.12-2.26 (m, 2H), 3.55-3.74 (m, 1H), 3.93-4.20 (m, 1H), 6.34 (b, 2H), 6.50-7.20 (m, 4H), 7.35 (t, 2H), 7.56 (bs, 2H), 8.04 (bs, 1H), 8.21 (bs, 1H), 9.72 (bs, 1H).

### EXAMPLE 118

### (R)-1-(3-(2-(6-Fluoroimidazo[1,2-a]pyridin-3-yl)pyrimidin-4-ylamino)piperidin-1-O-5-(4-fluorophenyl)pentan-1-one

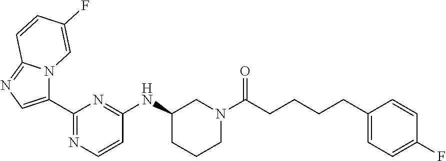 [SMILES: O=C(CCCCc1ccc(F)cc1)N1CCC[C@@H](Nc2ccnc(-c3cnc4ccc(F)cn34)n2)C1]

Obtained as a white solid (22 mg, 46%) from 5-(4-fluorophenyl)pentanoic acid (38 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 491 (M+1)⁺.

¹H NMR [symbol] (CD₃OD): 1.33 (m, 1H), 1.45 (m, 1H), 1.59-1.84 (m, 4H), 1.88 (m, 1H), 2.16 (m, 1H), 2.29 (t, 2H), 2.46 (t, 1H), 2.64 (t, 1H), 2.80 (dd, 1H), 3.10-3.24 (m, 1H), 3.46 (dd, 1H), 3.84 (d, 2H), 6.43 (d, 1H), 6.74-6.83 (m, 1H), 6.83-6.91 (m, 1H), 6.96 (t, 1H), 7.19 (dd, 1H), 7.37-7.50 (m, 1H), 7.68 (dt, 1H), 8.09-8.17 (m, 1H), 8.19 (d, 1H), 8.42 (s, 1H), 8.48 (s, 1H), 10.00 (bs, 1H).

### EXAMPLE 119

### (R)-2-(2-Chlorophenyl)-1-((R)-3-(2-(6-fluoroimidazo[1,2-a]pyridin-3-yl)pyrimidin-4-ylamino)piperidin-1-yl)-2-hydroxyethanone

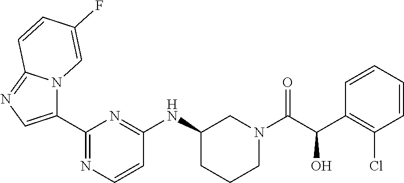 [SMILES: O=C([C@H](O)c1ccccc1Cl)N1CCC[C@@H](Nc2ccnc(-c3cnc4ccc(F)cn34)n2)C1]

Obtained as a white solid (19 mg, 40%) from (R)-2-(2-chlorophenyl)-2-hydroxyacetic acid (38 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 481 (M+1)⁺.

¹H NMR [symbol] (CD₃OD): 0.81-0.95 (m, 2H), 1.57 (m, 4H), 1.65-1.78 (m, 4H), 1.86-1.96 (m, 1H), 1.98-2.08 (m, 1H), 2.13 (m, 1H), 2.86 (dd, 1H), 3.00 (dd, 1H), 3.08-3.20 (m, 1H), 3.59 (d, 1H), 3.81 (d, 1H), 3.89-4.08 (m, 1H), 4.11-4.24 (m, 1H), 4.69-4.79 (m, 1H), 5.62-5.72 (m, 1H), 5.81 (s, 1H), 6.20-6.30 (m, 1H), 6.31-6.42 (m, 1H), 7.05-7.14 (m, 2H), 7.19 (t, 1H), 7.23-7.31 (m, 1H), 7.38 (d, 2H), 7.42-7.55 (m, 5H), 7.72 (ddd, 2H), 8.08 (d, 3H), 8.39-8.59 (m, 2H), 9.94-10.12 (m, 1H).

### EXAMPLE 120

### (R)-1-(3-(2-(6-Fluoroimidazo[1,2-a]pyridin-3-yl)pyrimidin-4-ylamino)piperidin-1-yl)-2-(methylamino)ethanone

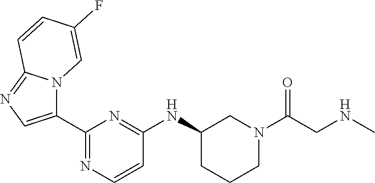 [SMILES: CNCC(=O)N1CCC[C@@H](Nc2ccnc(-c3cnc4ccc(F)cn34)n2)C1]

Obtained as a white solid (19 mg, 52%) from 2-(methylamino)acetic acid (36 mg, 0.192 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 384 (M+1)⁺.

¹H NMR [symbol] (CD₃OD): 1.54-1.83 (m, 2H), 1.83-1.97 (m, 1H), 2.03-2.22 (m, 1H), 2.23-2.30 (m, 1H), 2.42 (bs, 3H), 2.90 (dd, 1H), 3.12-3.25 (m, 1H), 3.33-3.43 (m, 1H), 3.49 (bs, 2H), 3.73 (d, 1H), 3.82 (dd, 1H), 4.59 (d, 1H), 6.39 (dd, 1H), 7.44 (dd, 1H), 7.68 (td, 1H), 8.03-8.23 (m, 1H), 8.42 (s, 1H), 8.47 (s, 1H), 9.88-10.11 (m, 1H).

### EXAMPLE 121

### (R)-1-(3-(2-(6-Fluoroimidazo[1,2-a]pyridin-3-yl)pyrimidin-4-ylamino)piperidin-1-yl)-2-(2-methoxyethoxy)ethanone

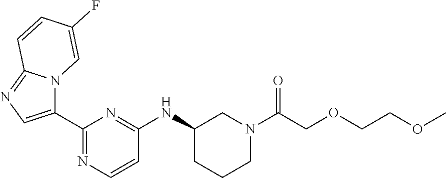 [SMILES: COCCOCC(=O)N1CCC[C@@H](Nc2ccnc(-c3cnc4ccc(F)cn34)n2)C1]

Obtained as a white solid (37 mg, 88%) from 2-(2-methoxyethoxy)acetic acid (22 μL, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 429 (M+1)⁺.

¹H NMR [symbol] (CD₃OD): 1.52-1.80 (m, 2H), 1.83-1.99 (m, 1H), 2.11-2.21 (m, 1H), 2.91-3.01 (m, 1H), 3.09-3.27 (m, 2H), 3.37 (s, 3H), 3.41-3.45 (m, 1H), 3.45-3.53 (m, 1H), 3.59 (dd, 1H), 3.65-3.71 (m, 1H), 3.79 (d, 1H), 3.89 (d, 1H), 4.28 (s, 2H), 4.50 (d, 1H), 6.31-6.48 (m, 1H), 7.45 (dd, 1H), 7.70 (td, 1H), 8.09-8.23 (m, 1H), 8.45 (d, 1H), 9.91-10.12 (m, 1H).

### EXAMPLE 122

### (R)-1-(3-(2-(6-Fluoroimidazo[1,2-a]pyridin-3-yl)pyrimidin-4-ylamino)piperidin-1-yl)-2-(2-methylthiazol-4-yl)ethanone

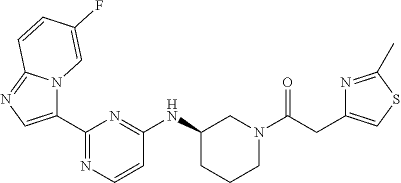 [SMILES: Cc1nc(CC(=O)N2CCC[C@@H](Nc3ccnc(-c4cnc5ccc(F)cn45)n3)C2)cs1]

Obtained as a white solid (29 mg, 66%) from 2-(2-methylthiazol-4-yl)acetic acid (31 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 452 (M+1)⁺.

¹H NMR [symbol] (CD₃OD): 1.57-1.77 (m, 4H), 1.87 (dt, 1H), 2.10-2.22 (m, 1H), 2.68 (s, 3H), 3.00 (dd, 1H), 3.76 (s, 2H), 3.92 (d, 2H), 4.01 (d, 1H), 4.51 (d, 1H), 6.39 (d, 1H), 7.00 (bs, 1H), 7.14 (s, 1H), 7.36-7.50 (m, 1H), 7.68 (td, 1H), 8.15 (d, 1H), 8.33-8.48 (m, 1H), 9.97 (d, 1H).

### EXAMPLE 123

### (R)-(3-(2-(6-Fluoroimidazo[1,2-a]pyridin-3-yl)pyrimidin-4-ylamino)piperidin-1-yl)(tetrahydro-2H-pyran-4-yl)methanone

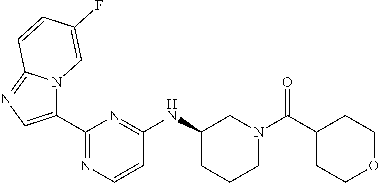 [SMILES: O=C(C1CCOCC1)N1CCC[C@@H](Nc2ccnc(-c3cnc4ccc(F)cn34)n2)C1]

Obtained as a white solid (21 mg, 51%) from tetrahydro-2H-pyran-4-carboxylic acid (25 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 425 (M+1)⁺.

¹H NMR [symbol] (CD₃OD): 1.53-1.99 (m, 6H), 2.10-2.26 (m, 2H), 2.77 (dd, 1H), 2.85-3.06 (m, 1H), 3.44-3.59 (m, 2H), 3.68-3.77 (m, 1H), 3.82-3.91 (m, 1H), 3.96 (t, 2H), 4.17 (bs, 1H), 4.67 (d, 1H), 6.43 (d, 1H), 7.46 (dt, 1H), 7.71 (ddd, 1H), 8.19 (d, 1H), 8.42 (s, 1H), 8.47 (s, 1H), 9.91-10.08 (m, 1H).

### EXAMPLE 124

### (R)-1-(3-(2-(6-Fluoroimidazo[1,2-a]pyridin-3-yl)pyrimidin-4-ylamino)piperidin-1-yl)-2-(2-methyl-1H-benzo[d]imidazol-1-yl)ethanone

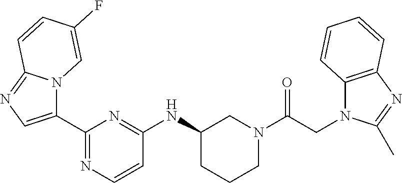 [SMILES: Cc1nc2ccccc2n1CC(=O)N1CCC[C@@H](Nc2ccnc(-c3cnc4ccc(F)cn34)n2)C1]

Obtained as a white solid (20 mg, 43%) from 2-(2-methyl-1H-benzo[d]imidazol-1-yl)acetic acid (37 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 485 (M+1)⁺.

¹H NMR [symbol] (CD₃OD): 1.64-1.79 (m, 1H), 2.02 (m, 1H), 2.14-2.31 (m, 1H), 2.41 (s, 1H), 2.52 (s, 3H), 2.94 (m, 1H), 3.54-3.72 (m, 1H), 3.87-4.07 (m, 1H), 4.56 (d, 1H), 5.14-5.36 (m, 2H), 6.35 (d, 1H), 6.54 (d, 1H), 7.08-7.16 (m, 1H), 7.21 (td, 1H), 7.31-7.51 (m, 1H), 7.52-7.58 (m, 1H), 7.65 (td, 1H), 8.11 (d, 1H), 8.25 (d, 1H), 8.40 (s, 1H), 8.47 (s, 1H), 9.85-10.11 (m, 1H).

### EXAMPLE 125

### Chroman-3-yl((R)-3-(2-(6-fluoroimidazo[1,2-a]pyridin-3-yl)pyrimidin-4-ylamino)piperidin-1-yl)methanone

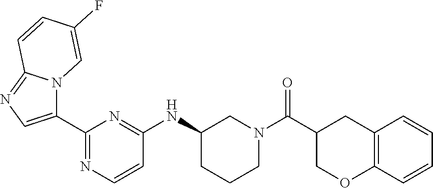 [SMILES: O=C(C1COc2ccccc2C1)N1CCC[C@@H](Nc2ccnc(-c3cnc4ccc(F)cn34)n2)C1]

Obtained as a white solid (35 mg, 77%) from chroman-3-carboxylic acid (34 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 473 (M+1)⁺.

¹H NMR [symbol] (CD₃OD): 1.58-2.02 (m, 4H), 2.08-2.26 (m, 2H), 2.76-3.14 (m, 2H), 3.46-3.72 (m, 1H), 3.74-4.10 (m, 4H), 4.27-4.46 (m, 1H), 6.44 (dd, 1H), 6.54-6.64 (m, 1H), 6.68-6.80 (m, 1H), 6.80-6.89 (m, 1H), 6.98-7.16 (m, 1H), 7.37-7.50 (m, 1H), 7.67 (d, 1H), 8.16 (dd, 1H), 8.28 (bs, 1H), 8.49 (s, 1H), 9.98-10.08 (m, 1H).

### EXAMPLE 126

### (R)—N-(2-(3-(2-(6-Fluoroimidazo[1,2-a]pyridin-3-yl)pyrimidin-4-ylamino)piperidin-1-yl)-2-oxoethyl)-N-methylbenzamide

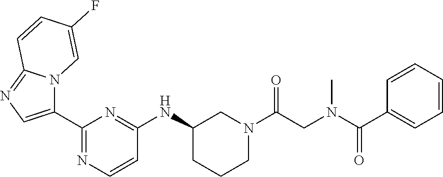 [SMILES: CN(CC(=O)N1CCC[C@@H](Nc2ccnc(-c3cnc4ccc(F)cn34)n2)C1)C(=O)c1ccccc1]

Obtained as a white solid (36 mg, 77%) from 2-(N-methylbenzamido)acetic acid (37 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 488 (M+1)⁺.

¹H NMR [symbol] (CD₃OD): 1.50-2.02 (m, 4H), 2.07-2.26 (m, 1H), 2.97 (s, 2H), 3.04 (s, 3H), 3.12 (s, 1H), 3.72-4.04 (m, 1H), 4.09-4.42 (m, 2H), 6.33-6.44 (m, 1H), 6.45-6.52 (m, 1H), 7.20-7.35 (m, 1H), 7.35-7.55 (m, 5H), 7.62-7.77 (m, 1H), 8.07-8.23 (m, 1H), 8.47 (bs, 1H), 10.00-10.12 (m, 1H).

### EXAMPLE 127

### (R)-2-(3-Chlorophenoxy)-1-(3-(2-(6-fluoroimidazo[1,2-a]pyridin-3-yl)pyrimidin-4-ylamino)piperidin-1-yl)ethanone

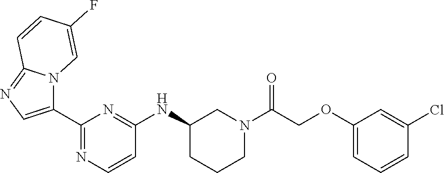 [SMILES: O=C(COc1cccc(Cl)c1)N1CCC[C@@H](Nc2ccnc(-c3cnc4ccc(F)cn34)n2)C1]

Obtained as a white solid (39 mg, 83%) from 2-(3-chlorophenoxy)acetic acid (36 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 481 (M+1)⁺.

¹H NMR [symbol] (CD₃OD): 1.56-1.85 (m, 4H), 1.87-2.01 (m, 2H), 2.11-2.19 (m, 2H), 2.20 (s, 2H), 2.98 (t, 2H), 3.45-3.57 (m, 2H), 3.7 (m, 2H), 3.73-3.93 (m, 2H), 4.47-4.67 (m, 2H), 6.37 (d, 1H), 6.44 (d, 1H), 6.52-6.61 (m, 1H), 6.61-6.72 (m, 2H), 6.81-7.01 (m, 4H), 7.03 (t, 1H), 7.15-7.32 (m, 1H), 7.35-7.50 (m, 2H), 7.63 (dd, 2H), 8.16 (d, 2H), 8.38 (s, 1H), 8.46 (s, 1H), 9.86-9.95 (m, 2H), 9.97-10.08 (m, 1H).

### EXAMPLE 128

### (R)-1-(3-(2-(6-Fluoroimidazo[1,2-a]pyridin-3-yl)pyrimidin-4-ylamino)piperidin-1-yl)-2,2-bis(hydroxymethyl)butan-1-one

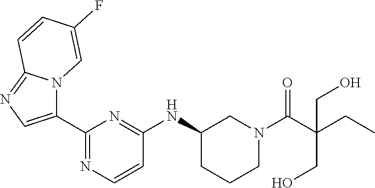 [SMILES: CCC(CO)(CO)C(=O)N1CCC[C@@H](Nc2ccnc(-c3cnc4ccc(F)cn34)n2)C1]

Obtained as a white solid (9 mg, 21%) from 2,2-bis(hydroxymethyl)butanoic acid (29 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 443 (M+1)⁺.

¹H NMR [symbol] (CD₃OD): 0.89 (t, 3H), 1.51-1.81 (m, 6H), 1.90 (dd, 1H), 2.07-2.23 (m, 1H), 3.69-3.92 (m, 7H), 3.97-4.10 (m, 1H), 4.20-4.38 (m, 2H), 6.38 (bs, 1H), 7.45 (ddd, 1H), 7.69 (dd, 1H), 8.15 (bs, 1H), 8.46 (s, 1H), 10.04 (bs, 1H).

### EXAMPLE 129

### (R)-1-(3-(2-(6-Fluoroimidazo[1,2-a]pyridin-3-yl)pyrimidin-4-ylamino)piperidin-1-yl)-2-(2H-tetrazol-5-yl)ethanone

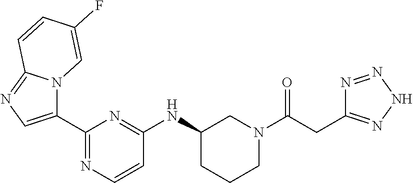 [SMILES: O=C(Cc1nn[nH]n1)N1CCC[C@@H](Nc2ccnc(-c3cnc4ccc(F)cn34)n2)C1]

Obtained as a white solid (41 mg, 98%) from 2-(2H-tetrazol-5-yl)acetic acid (25 mg, 0.19 mmol) and (R)-2-(6-fluoroimidazo[1,2-a]pyridin-3-yl)-N-(piperidin-3-yl)pyrimidin-4-amine (30 mg, 0.1 mmol, Preparation 9b) following the experimental procedure as described in Example 95.

LRMS (m/z): 423 (M+1)⁺.

¹H NMR [symbol] (CD₃OD): 1.52-1.95 (m, 4H), 2.02-2.22 (m, 1H), 3.33 (s, 2H), 3.72-3.87 (m, 1H), 3.87-4.29 (m, 4H), 4.35-4.46 (m, 1H), 6.42 (bs, 1H), 7.43 (d, 1H), 7.67 (dd, 1H), 8.14 (bs, 1H), 8.43 (d, 1H), 10.01 (bs, 1H).

### EXAMPLE 130

### N-(1-(1H-1,2,4-Triazol-3-yl)piperidin-3-yl)-2-(6-fluoroimidazo[1,2-a]pyridin-3-yl)pyrimidin-4-amine

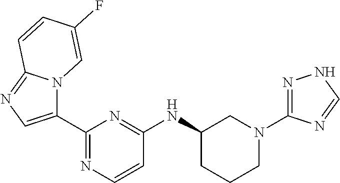 [SMILES: Fc1ccc2ncc(-c3nccc(N[C@@H]4CCCN(c5nc[nH]n5)C4)n3)n2c1]

(R)-2-(6-Fluoroimidazo[1,2-a]pyridin-3-yl)-N-(piperidin-3-yl)pyrimidin-4-amine (42 mg, 0.13 mmol, Preparation 9b) and 3-bromo-1H-1,2,4-triazole (10 mg, 0.07 mmol, prepared as described in the J. Med. Chem. 2004, 47(19), 4645-4648) are heated overnight at 150° C. The crude reaction mixture is directly purified by reverse phase chromatography (C-18 silica from Waters, water/1:1 acetonitrile-methanol as eluents [0.1% v/v formic acid buffered] 0% to 100%) to obtain the title compound (4 mg, 15%) as a solid.

LRMS (m/z): 380 (M+1)⁺.

¹H-NMR δ (CDCl₃): 1.8 (m, 2H), 1.9 (m, 1H), 2.0 (m, 1H), 3.2 (m, 1H), 3.3 (m, 1H), 3.7 (m, 1H), 4.1 (m, 2H), 5.2 (bs, 1H), 6.1 (m, 1H), 7.2 (m, 1H), 7.6 (dd, 1H), 7.8 (m, 1H), 8.1 (m, 1H), 8.5 (s, 1H), 9.8 (m, 1H).

### EXAMPLE 131

### (R)-2-(3-(2-(6-Fluoroimidazo[1,2-a]pyridin-3-yl)pyrimidin-4-ylamino)piperidin-1-yl)thiazole-5-carbonitrile

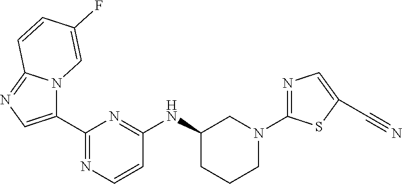 [SMILES: N#Cc1cnc(N2CCC[C@@H](Nc3ccnc(-c4cnc5ccc(F)cn45)n3)C2)s1]

Obtained as a white solid (69 mg, 62%) from 2-bromothiazole-5-carbonitrile (50 mg, 0.26 mmol) and (R)-2-(6-fluoroimidazo[1,2-a]pyridin-3-yl)-N-(piperidin-3-yl)pyrimidin-4-amine (125 mg, 0.4 mmol, Preparation 9b) following the experimental procedure as described in Example 130.

LRMS (m/z): 421 (M+1)⁺.

¹H-NMR δ (CDCl₃): 1.8 (m, 2H), 2.0 (m, 2H), 2.1 (m, 1H), 3.3 (dd, 1H), 3.4 (m, 1H), 3.7 (m, 1H), 4.3 (dd, 1H), 5.3 (d, 1H), 6.2 (d, 1H), 7.2 (m, 1H), 7.6 (m, 2H), 8.2 (d, 1H), 8.6 (s, 1H), 9.9 (dd, 1H).

### EXAMPLE 132

### (R)-1-((R)-3-(2-(6-Fluoroimidazo[1,2-a]pyridin-3-yl)pyrimidin-4-ylamino)piperidin-1-yl)-2,3-dihydroxypropan-1-one

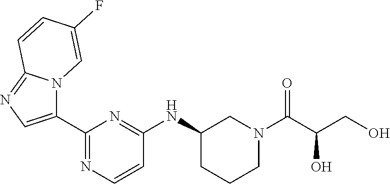 [SMILES: O=C([C@H](O)CO)N1CCC[C@@H](Nc2ccnc(-c3cnc4ccc(F)cn34)n2)C1]

Obtained as a white solid (66 mg, 60%) from (R)-2,2-dimethyl-1,3-dioxolan-4-carboxylic acid (120 mg, 0.82 mmol) and (R)-2-(6-fluoroimidazo[1,2-a]pyridin-3-yl)-N-(piperidin-3-yl)pyrimidin-4-amine (220 mg, 0.67 mmol, Preparation 9b) following the experimental procedure as described in Example 95 followed by treatment with acetic acid.

LRMS (m/z): 401 (M+1)⁺.

¹H NMR δ (DMSO-d₆): 1.41-1.69 (m, 4H), 1.72-1.86 (m, 1H), 2.00-2.18 (m, 1H), 2.59-2.76 (m, 1H), 2.99-3.19 (m, 1H), 3.42-3.67 (m, 2H), 3.76-4.45 (m, 2H), 4.53-4.79 (m, 1H), 4.80-5.01 (m, 1H), 6.40 (bs, 1H), 7.52 (t, 2H), 7.80 (dd, 1H), 8.20 (bs, 1H), 8.36-8.66 (m, 1H), 9.96 (bs, 1H).

### EXAMPLE 133

### (S)-1-((R)-3-(2-(6-Fluoroimidazo[1,2-a]pyridin-3-yl)pyrimidin-4-ylamino)piperidin-1-yl)-2,3-dihydroxypropan-1-one

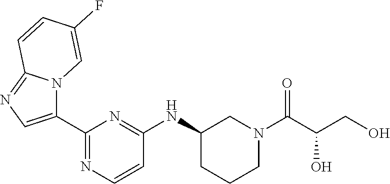 [SMILES: O=C([C@@H](O)CO)N1CCC[C@@H](Nc2ccnc(-c3cnc4ccc(F)cn34)n2)C1]

Obtained as a white solid (68 mg, 53%) from (S)-2,2-dimethyl-1,3-dioxolan-4-carboxylic acid (59 mg, 0.39 mmol) and (R)-2-(6-fluoroimidazo[1,2-a]pyridin-3-yl)-N-(piperidin-3-yl)pyrimidin-4-amine (100 mg, 0.32 mmol, Preparation 9b) following the experimental procedure as described in Example 132.

LRMS (m/z): 401 (M+1)⁺.

¹H NMR δ (DMSO-d₆): 1.58 (m, 2H), 1.70-1.84 (m, 1H), 2.01-2.16 (m, 1H), 2.58-2.78 (m, 1H), 2.90-3.21 (m, 1H), 3.37-3.66 (m, 2H), 3.76-4.19 (m, 2H), 4.26-4.50 (m, 1H), 4.55-4.79 (m, 2H), 4.88 (bs, 1H), 6.40 (d, 1H), 7.52 (dd, 2H), 7.80 (dd, 1H), 8.10-8.30 (m, 1H), 8.37-8.62 (m, 1H), 9.97 (bs, 1H).

### EXAMPLE 134

### (R)-3-(3-(2-(6-Fluoroimidazo[1,2-a]pyridin-3-yl)-5-methylpyrimidin-4-ylamino) pyrrolidin-1-yl)-3-oxopropanenitrile

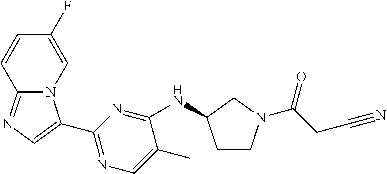 [SMILES: Cc1cnc(-c2cnc3ccc(F)cn23)nc1N[C@@H]1CCN(C(=O)CC#N)C1]

Obtained as a white solid (30 mg, 32%) from (R)-2-(6-fluoroimidazo[1,2-a]pyridin-3-yl)-5-methyl-N-(pyrrolidin-3-yl)pyrimidin-4-amine (78 mg, 0.25 mmol, Preparation 48b) and 3-[(2,5-dioxopyrrolidin-1-yl)oxy]-3-oxopropanenitrile (68 mg, 0.37 mmol, prepared as described in BE875054(A1)) following the experimental procedure as described in Example 46.

LRMS (m/z): 380 (M+1)⁺.

¹H NMR [symbol] (CDCl₃): 1.11-1.49 (m, 2H), 2.12 (s, 3H), 2.35 (m, 2H), 3.43-3.78 (m, 3H), 3.81-4.06 (m, 1H), 4.72-5.05 (m, 1H), 5.58-6.03 (m, 1H), 7.24 (bs, 1H), 7.67 (bs, 1H), 8.08 (d, 1H), 8.47 (s, 1H), 9.99 (bs, 1H).

### EXAMPLE 135

### (S)-2-(6-Fluoroimidazo[1,2-a]pyridin-3-yl)-N-(1-(5-fluoropyridin-2-yl)ethyl)-5-methylpyrimidin-4-amine

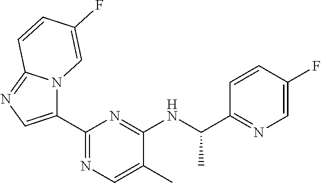 [SMILES: Cc1cnc(-c2cnc3ccc(F)cn23)nc1N[C@@H](C)c1ccc(F)cn1]

Obtained as a white solid (20 mg, 18%) from 2-(6-fluoroimidazo[1,2-a]pyridin-3-yl)-5-methylpyrimidin-4-ol (75 mg, 0.31 mmol, Preparation 19b) and (S)-1-(5-fluoropyridin-2-yl)ethanamine hydrochloride (108 mg, 0.61 mmol, prepared as described in WO2006/82392) following the experimental procedure as described in Preparation 5a.

LRMS (m/z): 367 (M+1)⁺.

¹H NMR [symbol] (CDCl₃): 1.66 (d, 3H), 2.17 (s, 3H), 5.50 (t, 1H), 5.94 (d, 1H), 7.20 (ddd, 1H), 7.38-7.44 (m, 2H), 7.65 (dd, 1H), 8.08 (s, 1H), 8.45 (s, 1H), 8.48 (s, 1H), 9.90 (dd, 1H).

### EXAMPLE 136

### 2-(6-Fluoroimidazo[1,2-a]pyridin-3-yl)-N⁴(5-fluoropyridin-2-yl)methyl)pyrimidine-4,5-diamine

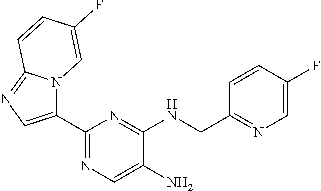 [SMILES: Nc1cnc(-c2cnc3ccc(F)cn23)nc1NCc1ccc(F)cn1]

Palladium on carbon (10%, 48 mg, 0.05 mmol) was added to a suspension of 2-(6-fluoroimidazo[1,2-a]pyridin-3-yl)-N-((5-fluoropyridin-2-yl)methyl)-5-nitropyrimidin-4-amine (113 mg, 0.29 mmol, Preparation 49c) in ethanol (15 mL) and the mixture was stirred under a hydrogen atmosphere at ambient temperature for 2 hours. The mixture was filtered through Celite® and the filter cake was washed with ethanol. The combined filtrate and washings were evaporated to give the title compound (88 mg, 96%) as a pale green solid.

LRMS (m/z): 354 (M+1)⁺.

¹H NMR δ (DMSO-d₆): 4.83 (d, 2H), 5.14 (s, 2H), 7.41 (dd, 1H), 7.47-7.61 (m, 2H), 7.71 (td, 2H), 7.79 (s, 1H), 8.15 (s, 1H), 8.56 (d, 1H), 9.65 (dd, 1H).

### EXAMPLE 137

### 2-(6-Fluoroimidazo[1,2-a]pyridin-3-yl)-N⁴-(5,6,7,8-tetrahydroquinolin-5-yl)pyrimidine-4,5-diamine

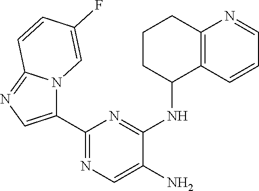 [SMILES: Nc1cnc(-c2cnc3ccc(F)cn23)nc1NC1CCCc2ncccc21]

Obtained as a pale green solid (94%) from N-(2-(6-fluoroimidazo[1,2-a]pyridin-3-yl)-5-nitropyrimidin-4-yl)-5,6,7,8-tetrahydroquinolin-5-amine (Preparation 50) following the experimental procedure as described in Example 136.

LRMS (m/z): 376 (M+1)⁺.

¹H NMR (300 MHz, DMSO-d₆) δ ppm 1.90-2.16 (m, 4H), 2.93 (m, 2H), 5.08 (br s, 2H), 5.58 (dd, 1H), 6.99 (d, 1H), 7.20 (dd, 1H), 7.42 (ddd, 1H), 7.67 (d, 1H), 7.74 (dd, 1H), 7.76 (s, 1H), 8.22 (s, 1H), 8.41 (dd, 1H), 9.91 (dd, 1H).

### EXAMPLE 138

### (R)-2-(6-Fluoroimidazo[1,2-a]pyridin-3-yl)-N⁴-(3-methylbutan-2-yl)pyrimidine-4,5-diamine

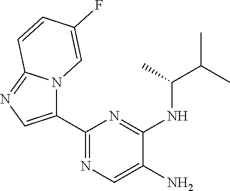 [SMILES: CC(C)[C@@H](C)Nc1nc(-c2cnc3ccc(F)cn23)ncc1N]

Obtained as a pale brown solid (114 mg, 99%) from (R)-2-(6-fluoroimidazo[1,2-a]pyridin-3-yl)-N-(3-methylbutan-2-yl)-5-nitropyrimidin-4-amine (Preparation 51) following the experimental procedure as described in Example 136.

LRMS (m/z): 315 (M+1)⁺.

¹H NMR δ (DMSO-d₆): 0.94 (dd, 3H), 1.01 (t, 3H), 1.20 (dd, 3H), 1.91-2.11 (m, 1H), 4.12-4.28 (m, 1H), 5.02-5.16 (m, 2H), 6.35 (d, 1H), 7.35-7.50 (m, 1H), 7.65-7.71 (m, 1H), 7.75 (dd, 1H), 8.16-8.25 (m, 1H), 9.95 (dt, 1H).

### EXAMPLE 139

### 2-(6-Fluoroimidazo[1,2-a]pyridin-3-yl)-N⁴-(1-methoxypropan-2-yl)pyrimidine-4,5-diamine

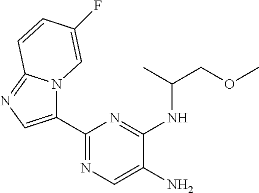 [SMILES: COCC(C)Nc1nc(-c2cnc3ccc(F)cn23)ncc1N]

Obtained as a white solid (105 mg, 100%) from 2-(6-fluoroimidazo[1,2-a]pyridin-3-yl)-N-(1-methoxypropan-2-yl)-5-nitropyrimidin-4-amine (115 mg, 0.33 mmol, Preparation 52) following the experimental procedure as described in Example 136.

LRMS (m/z): 317 (M+1)⁺.

¹H NMR δ (DMSO-d₆): 1.28 (d, 3H), 3.32 (s, 3H), 3.38-3.42 (m, 1H), 3.49-3.62 (m, 1H), 4.26-4.57 (m, 1H), 5.07 (s, 2H), 6.45 (d, 1H), 7.42 (dd, 1H), 7.74 (dd, 2H), 8.19 (s, 1H), 9.91 (dd, 1H).

### EXAMPLE 140

### 2-((1r,4r)-4-(5-Amino-2-(6-fluoroimidazo[1,2-a]pyridin-3-yl)pyrimidin-4-ylamino) cyclohexyl)acetonitrile

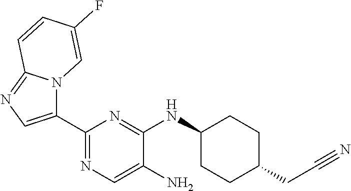 [SMILES: N#CC[C@H]1CC[C@H](Nc2nc(-c3cnc4ccc(F)cn34)ncc2N)CC1]

Obtained as a pale green solid (92%) from 24(1r,4r)-4-(2-(6-fluoroimidazo[1,2-a]pyridin-3-yl)-5-nitropyrimidin-4-ylamino)cyclohexyl)acetonitrile (Preparation 53) following the experimental procedure as described in Example 136.

LRMS (m/z): 366 (M+1)⁺.

¹H NMR [symbol] (CDCl₃): 1.26-1.47 (m, 4H), 1.79 (m, 1H), 2.05 (m, 2H), 2.36 (d, 4H), 3.06 (br s, 2H), 4.06 (m, 1H), 4.85 (d, 1H), 7.21 (ddd, 1H), 7.66 (dd, 1H), 7.89 (s, 1H), 8.43 (s, 1H), 9.95 (dd, 1H).

### EXAMPLE 141

### (1r,4r)-4-(5-Amino-2-(6-fluoroimidazo[1,2-a]pyridin-3-yl)pyrimidin-4-ylamino)-1-methylcyclohexanol

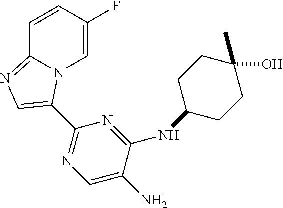

Obtained as an off-white solid (51%) from (1r,4r)-4-(2-(6-fluoroimidazo[1,2-a]pyridin-3-yl)-5-nitropyrimidin-4-ylamino)-1-methylcyclohexanol (110 mg, 0.23 mmol, Preparation 54) following the experimental procedure as described in Example 136.

LRMS (m/z): 357 (M+1)⁺.

¹H-NMR δ (DMSO-d₆): 1.2 (s, 3H), 1.4-1.7 (m, 6H), 2.0 (m, 2H), 4.0 (bs, 1H), 4.4 (s, 1H), 5.0 (s, 2H), 6.4 (d, 1H), 7.4 (m, 1H), 7.7 (s, 1H), 7.7 (dd, 1H), 8.2 (s, 1H), 9.9 (dd, 1H).

### EXAMPLE 142

### 2-(6-Fluoroimidazo[1,2-a]pyridin-3-yl)-N⁴-(1-(5-fluoropyridin-2-yl)-2-methoxyethyl)pyrimidine-4,5-diamine

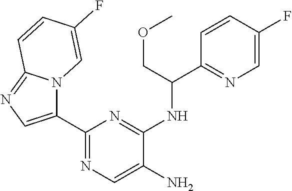 [SMILES: COCC(Nc1nc(-c2cnc3ccc(F)cn23)ncc1N)c1ccc(F)cn1]

Obtained as a pale brown solid (87%) from 2-(6-fluoroimidazo[1,2-a]pyridin-3-yl)-N-(1-(5-fluoropyridin-2-yl)-2-methoxyethyl)-5-nitropyrimidin-4-amine (142 mg, 0.33 mmol, Preparation 55) following the experimental procedure as described in Example 136 followed by purification of the crude product by flash chromatography (1-5% methanol in dichloromethane).

LRMS (m/z): 398 (M+1)⁺.

¹H NMR δ (DMSO-d₆): 3.35 (s, 3H), 3.75-3.93 (m, 1H), 5.28 (s, 2H), 5.46-5.63 (m, 1H), 7.14 (d, 1H), 7.42 (dd, 1H), 7.55-7.63 (m, 1H), 7.66-7.81 (m, 4H), 8.02-8.18 (m, 1H), 8.53-8.64 (m, 1H), 9.56-9.74 (m, 1H).

### EXAMPLE 143

### (S)-2-(6-Fluoroimidazo[1,2-a]pyridin-3-yl)-4-(1-(5-fluoropyridin-2-yl)ethylamino) pyrimidine-5-carboxylic acid

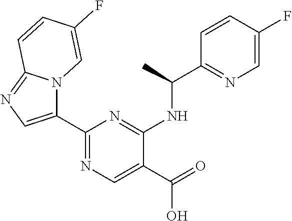 [SMILES: C[C@H](Nc1nc(-c2cnc3ccc(F)cn23)ncc1C(=O)O)c1ccc(F)cn1]

A solution of lithium hydroxide monohydrate (61 mg, 1.45 mmol) in water (2 mL) was added to a solution of (S)-ethyl 2-(6-fluoroimidazo[1,2-a]pyridin-3-yl)-4-(1-(5-fluoro pyridin-2-yl)ethylamino)pyrimidine-5-carboxylate (62 mg, 0.15 mmol, Preparation 57) in a mixture of methanol (10 mL) and THF (8 mL) and the resulting mixture was stirred at room temperature for 1 hour. The solvents were evaporated under reduced pressure and the residue was diluted with water. After addition of 2N aqueous hydrochloric acid solution until pH=4 a solid precipitated, which was filtered and dried in an oven under vacuum at 45° C. to yield 54 mg (93%) of the title compound.

LRMS (m/z): 397 (M+1)⁺.

¹H NMR δ (DMSO-d₆): 1.61 (d, 3H), 3.13-3.53 (m, 1H), 5.36-5.70 (m, 1H), 7.36-7.98 (m, 4H), 8.38-8.66 (m, 1H), 8.86 (bs, 1H), 9.14 (bs, 1H), 9.70 (bs, 1H), 13.23-13.66 (m, 1H).

### EXAMPLE 144

### (S)-2-(6-Fluoroimidazo[1,2-a]pyridin-3-yl)-4-(1-(5-fluoropyridin-2-yl)ethylamino) pyrimidine-5-carboxamide

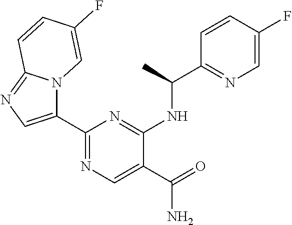 [SMILES: C[C@H](Nc1nc(-c2cnc3ccc(F)cn23)ncc1C(N)=O)c1ccc(F)cn1]

N-(3-Dimethylaminopropyl)-N′-ethylcarbodiimide hydrochloride (138 mg, 0.72 mmol) and 1-hydroxybenzotriazole (97 mg, 0.72 mmol) were added to a solution of (S)-2-(6-Fluoroimidazo[1,2-a]pyridin-3-yl)-4-(1-(5-fluoropyridin-2-yl)ethylamino)pyrimidine-5-carboxylic acid (190 mg, 0.48 mmol, Example 143) in N,N′-dimethylformamide (7 mL) and the resulting mixture was stirred half an hour at room temperature. Aqueous ammonium hydroxide solution (28 μL, 0.71 mmol) was then added and the reaction mixture was stirred overnight at room temperature. Water was added and a solid precipitated, which was filtered and washed with water. Once dried at 50° C. under vacuum, 140 mg (74%) of the title compound were obtained.

LRMS (m/z): 396 (M+1)⁺.

¹H-NMR δ (DMSO-d₆): 1.6 (d, 3H), 5.5 (m, 1H), 7.5 (m, 2H), 7.6 (m, 1H), 7.8 (dd, 1H), 8.4 (s, 1H), 8.5 (d, 1H), 8.8 (s, 1H), 9.7 (bs, 2H).

### EXAMPLE 145

### (S)-2-(6-Fluoroimidazo[1,2-a]pyridin-3-yl)-4-((1-(5-fluoropyridin-2-yl)ethyl)(methyl)amino)pyrimidine-5-carboxylic acid

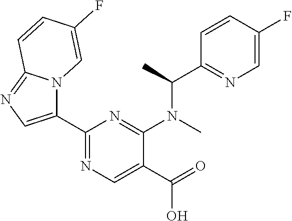 [SMILES: C[C@@H](c1ccc(F)cn1)N(C)c1nc(-c2cnc3ccc(F)cn23)ncc1C(=O)O]

Sodium hydride (60% suspension in hexane, 38 mg, 0.95 mmol) was added to a solution of (S)-ethyl 2-(6-fluoroimidazo[1,2-a]pyridin-3-yl)-4-(1-(5-fluoropyridin-2-yl)ethylamino)pyrimidine-5-carboxylate (200 mg, 0.47 mmol, Preparation 57) in N,N′-dimethylformamide (4 mL) and the resulting mixture was stirred at room temperature for 15 minutes. Methyl iodide (44 μL, 0.71 mmol) was then added and the mixture was heated at 60° C. overnight. After cooling to room temperature, the reaction mixture was poured onto water and stirred for half an hour. The solid precipitated was filtered, dried and purified by reverse phase chromatography (C-18 silica from Waters, water/1:1 acetonitrile-methanol as eluents [0.1% v/v formic acid buffered] 0% to 100%) to yield the title compound (11 mg, 5%).

LRMS (m/z): 411 (M+1)⁺.

¹H-NMR δ (CDCl₃): 1.8 (m, 3H), 2.9 (s, 3H), 6.2 (q, 1H), 7.3 (m, 1H), 7.4 (m, 2H), 7.8 (dd, 1H), 8.4 (s, 1H), 8.5 (s, 1H), 8.9 (s, 1H), 9.8 (dd, 1H).

### EXAMPLE 146

### 2-(6-Fluoroimidazo[1,2-a]pyridin-3-yl)-4-(pyridin-3-ylmethylamino)pyrimidine-5-carboxylic acid

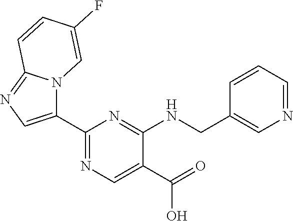 [SMILES: O=C(O)c1cnc(-c2cnc3ccc(F)cn23)nc1NCc1cccnc1]

Obtained as a solid (57%) from ethyl 2-(6-fluoroimidazo[1,2-a]pyridin-3-yl)-4-(pyridin-3-ylmethylamino)pyrimidine-5-carboxylate (520 mg, 1.33 mmol, Preparation 58) following the experimental procedure as described in Example 143.

LRMS (m/z): 365 (M+1)⁺.

¹H-NMR δ (DMSO-d₆): 4.8 (d, 2H), 7.3 (dd, 1H), 7.5 (t, 1H), 7.8 (m, 2H), 8.4 (d, 1H), 8.5 (s, 1H), 8.6 (s, 1H), 8.8 (s, 1H), 9.0 (s, 1H), 9.6 (d, 1H).

### EXAMPLE 147

### 2-(6-Fluoroimidazo[1,2-a]pyridin-3-yl)-4-(pyridin-3-ylmethylamino)pyrimidine-5-carboxamide

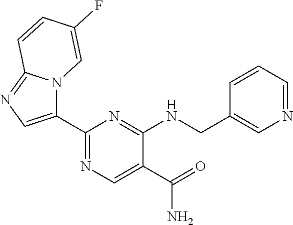 [SMILES: NC(=O)c1cnc(-c2cnc3ccc(F)cn23)nc1NCc1cccnc1]

Obtained as a solid (24%) from 2-(6-fluoroimidazo[1,2-a]pyridin-3-yl)-4-(pyridin-3-ylmethylamino)pyrimidine-5-carboxylic acid (230 mg, 0.63 mmol, Example 146) following the experimental procedure as described in Example 144.

LRMS (m/z): 364 (M+1)⁺.

¹H-NMR δ (DMSO-d₆): 4.8 (d, 2H), 7.3 (dd, 1H), 7.5 (m, 1H), 7.6 (bs, 1H), 7.8 (m, 2H), 8.2 (bs, 1H), 8.4 (dd, 1H), 8.5 (s, 1H), 8.6 (d, 1H), 8.8 (s, 1H), 9.6 (t, 1H), 9.7 (dd, 1H).

### EXAMPLE 148

### 2-(6-Fluoroimidazo[1,2-a]pyridin-3-yl)-4-((5-fluoropyridin-2-yl)methylamino)pyrimidine-5-carboxylic acid

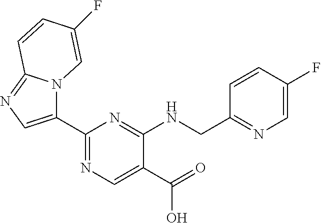 [SMILES: O=C(O)c1cnc(-c2cnc3ccc(F)cn23)nc1NCc1ccc(F)cn1]

Obtained as a solid (25%) from ethyl 2-(6-fluoroimidazo[1,2-a]pyridin-3-yl)-4((5-fluoropyridin-2-yl)methylamino)pyrimidine-5-carboxylate (280 mg, 0.68 mmol, Preparation 59) following the experimental procedure as described in Example 143.

LRMS (m/z): 383 (M+1)⁺.

¹H-NMR δ (DMSO-d₆): 4.9 (d, 2H), 7.5 (m, 2H), 7.7 (m, 1H), 7.8 (dd, 1H), 8.5 (s, 1H), 8.5 (d, 1H), 8.8 (s, 1H), 9.4 (s, 1H), 9.6 (dd, 1H), 13.4 (bs, 1H).

### EXAMPLE 149

### 2-(6-Fluoroimidazo[1,2-a]pyridin-3-yl)-44(5-fluoropyridin-2-yl)methylamino) pyrimidine-5-carboxamide

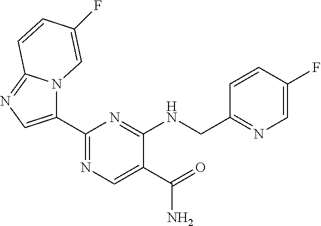 [SMILES: NC(=O)c1cnc(-c2cnc3ccc(F)cn23)nc1NCc1ccc(F)cn1]

Obtained as a solid (37%) from 2-(6-fluoroimidazo[1,2-a]pyridin-3-yl)-4-((5-fluoropyridin-2-yl)methylamino)pyrimidine-5-carboxylic acid (38 mg, 0.10 mmol, Example 148) following the experimental procedure as described in Example 144.

LRMS (m/z): 382 (M+1)⁺.

¹H-NMR δ (DMSO-d₆): 4.9 (d, 2H), 7.5 (dd, 1H), 7.7 (m, 2H), 7.9 (dd, 1H), 8.3 (d, 1H), 8.5 (d, 1H), 8.6 (s, 1H), 8.8 (s, 1H).

### EXAMPLE 150

### (S)-2-(6-Fluoroimidazo[1,2-a]pyridin-3-yl)-4-(1-(4-fluorophenyl)butylamino) pyrimidine-5-carboxylic acid

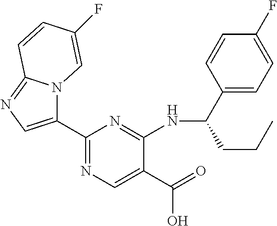 [SMILES: CCC[C@H](Nc1nc(-c2cnc3ccc(F)cn23)ncc1C(=O)O)c1ccc(F)cc1]

Obtained as a solid (52%) from (S)-ethyl 2-(6-fluoroimidazo[1,2-a]pyridin-3-yl)-4-(1-(4-fluorophenyl)butylamino)pyrimidine-5-carboxylate (470 mg, 0.71 mmol, Preparation 60) following the experimental procedure as described in Example 143.

LRMS (m/z): 424 (M+1)⁺.

¹H NMR δ (DMSO-d₆): 0.93 (t, 3H), 1.21-1.51 (m, 2H), 1.77-2.06 (m, 2H), 5.19-5.38 (m, 1H), 7.16 (t, 2H), 7.50 (dd, 1H), 7.59 (dd, 1H), 7.84 (dd, 1H), 8.49 (s, 1H), 8.84 (s, 1H), 9.02 (bs, 1H), 9.68 (d, 1H), 13.51 (bs, 1H).

### EXAMPLE 151

### (S)-2-(6-Fluoroimidazo[1,2-a]pyridin-3-yl)-4-(1-(4-fluorophenyl)butylamino) pyrimidine-5-carboxamide

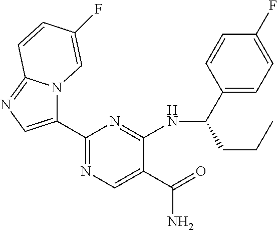 [SMILES: CCC[C@H](Nc1nc(-c2cnc3ccc(F)cn23)ncc1C(N)=O)c1ccc(F)cc1]

Obtained as a solid (37%) from (S)-2-(6-fluoroimidazo[1,2-a]pyridin-3-yl)-4-(1-(4-fluorophenyl)butylamino)pyrimidine-5-carboxylic acid (160 mg, 0.38 mmol, Example 150) following the experimental procedure as described in Example 144.

LRMS (m/z): 423 (M+1)⁺.

¹H-NMR δ (CDCl₃): 1.0 (t, 3H), 1.3-1.5 (m, 2H), 1.8-2.0 (m, 2H), 5.2 (m, 1H), 5.7 (bs, 2H), 7.0 (m, 2H), 7.2 (m, 1H), 7.4 (m, 2H), 7.7 (dd, 1H), 8.5 (s, 1H), 8.5 (s, 1H), 9.4 (d, 1H), 9.7 (dd, 1H).

### EXAMPLE 152

### 4-((1r,4r)-4-(Cyanomethyl)cyclohexylamino)-2-(6-fluoroimidazo[1,2-a]pyridin-3-yl)pyrimidine-5-carboxylic acid

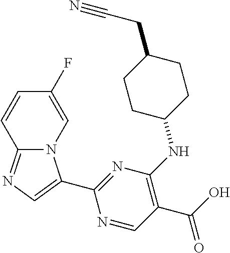

Obtained as a solid (72%) from ethyl 4-((1r,4r)-4-(cyanomethyl)cyclohexylamino)-2-(6-fluoroimidazo[1,2-a]pyridin-3-yl)pyrimidine-5-carboxylate (90 mg, 0.19 mmol, Preparation 61) following the experimental procedure as described in Example 143.

LRMS (m/z): 395 (M+1)⁺.

¹H-NMR δ (DMSO-d₆): 1.1-1.5 (m, 4H), 1.7 (m, 1H), 1.9 (d, 2H), 2.2 (d, 2H), 2.5 (d, 2H), 4.0 (m, 1H), 7.6 (m, 1H), 7.9 (m, 1H), 8.4 (d, 1H), 8.5 (s, 1H), 8.8 (s, 1H), 9.9 (m, 1H), 13.4 (s, 1H).

### EXAMPLE 153

### 4-((1r,4r)-4-(Cyanomethyl)cyclohexylamino)-2-(6-fluoroimidazo[1,2-a]pyridin-3-yl)pyrimidine-5-carboxamide

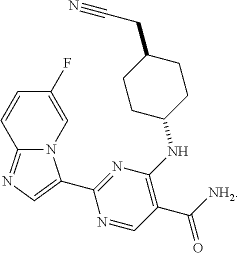

Obtained as a solid (59%) from 4-((1r,4r)-4-(cyanomethyl)cyclohexylamino)-2-(6-fluoroimidazo[1,2-a]pyridin-3-yl)pyrimidine-5-carboxylic acid (34 mg, 0.09 mmol, Example 152) following the experimental procedure as described in Example 144.

LRMS (m/z): 394 (M+1)⁺.

¹H-NMR δ (CDCl₃): 1.4 (m, 4H), 1.8 (s, 1H), 2.0 (d, 2H), 2.3 (d, 4H), 4.1 (m, 1H), 5.6 (m, 2H), 7.3 (m, 1H), 7.7 (m, 1H), 8.5 (s, 1H), 8.6 (s, 1H), 8.7 (m, 1H), 9.9 (s, 1H).

### EXAMPLE 154

### ((R)-2,2-Dimethyl-1,3-dioxolan-4-yl)((R)-3-(2-(6-fluoroimidazo[1,2-a]pyridin-3-yl)-5-methylpyrimidin-4-ylamino)piperidin-1-yl)methanone

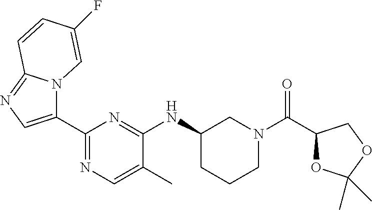 [SMILES: Cc1cnc(-c2cnc3ccc(F)cn23)nc1N[C@@H]1CCCN(C(=O)[C@H]2COC(C)(C)O2)C1]

Obtained as a white solid (160 mg, 52%) from (R)-2,2-dimethyl-1,3-dioxolan-4-carboxylic acid (120 mg, 0.82 mmol) and (R)-2-(6-fluoroimidazo[1,2-a]pyridin-3-yl)-5-methyl-N-(piperidin-3-yl)pyrimidin-4-amine (220 mg, 0.67 mmol, Preparation 20b) following the experimental procedure as described in Example 95 followed by purification by reverse phase chromatography (C-18 silica from Waters, water/1:1 acetonitrile-methanol as eluents [0.1% v/v formic acid buffered] 0% to 100%).

LRMS (m/z): 455 (M+1)⁺.

¹H-NMR δ (CDCl₃): 1.1-1.4 (m, 6H), 1.7 (m, 2H), 2.0 (m, 5H), 3.0 (m, 1H), 3.5-5 (m, 8H), 7.2 (m, 1H), 7.7 (m, 1H), 8.1 (m, 1H), 8.5 (m, 1H), 9.9 (dd, 1H).

### EXAMPLE 155

### (R)-1-((R)-3-(2-(6-Fluoroimidazo[1,2-a]pyridin-3-yl)-5-methylpyrimidin-4-ylamino) piperidin-1-yl)-2,3-dihydroxypropan-1-one

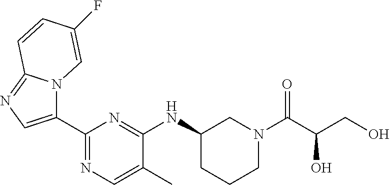 [SMILES: Cc1cnc(-c2cnc3ccc(F)cn23)nc1N[C@@H]1CCCN(C(=O)[C@H](O)CO)C1]

A solution of ((R)-2,2-dimethyl-1,3-dioxolan-4-yl)((R)-3-(2-(6-fluoroimidazo[1,2-a]pyridin-3-yl)-5-methylpyrimidin-4-ylamino)piperidin-1-yl)methanone (120 mg, 0.26 mmol, Example 154) in a mixture of glacial acetic acid (1.3 mL) and water (2.5 mL) was heated at 120° C. for 1.5 hours. The solvents were evaporated and the crude product was purified by reverse phase chromatography (C-18 silica from Waters, water/1:1 acetonitrile-methanol as eluents [0.1% v/v formic acid buffered] 0% to 100%) to yield the title compound (65 mg, 60%) as a white solid.

LRMS (m/z): 415 (M+1)⁺.

¹H-NMR δ (CDCl₃): 1.6-1.9 (m, 2H), 2.0 (m, 4H), 3.6 (m, 2H), 3.7-4.1 (m, 6H), 4.4 (m, 2H), 5.0 (m, 1H), 7.2 (t, 1H), 7.7 (dd, 1H), 8.0 (s, 1H), 8.5 (m, 1H), 9.9 (d, 1H).

### EXAMPLE 156

### (R)-3-(3-(5-Amino-2-(6-fluoroimidazo[1,2-a]pyridin-3-yl)pyrimidin-4-ylamino) piperidin-1-yl)-3-oxopropanenitrile

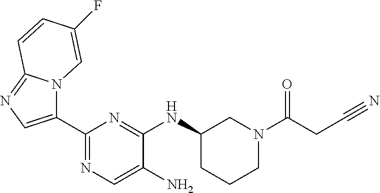 [SMILES: N#CCC(=O)N1CCC[C@@H](Nc2nc(-c3cnc4ccc(F)cn34)ncc2N)C1]

A solution of (R)-3-(3-(2-(6-fluoroimidazo[1,2-a]pyridin-3-yl)-5-nitropyrimidin-4-ylamino) piperidin-1-yl)-3-oxopropanenitrile (60 mg, 0.14 mmol, Preparation 62c) and tin dichloride dihydrate (130 mg, 0.58 mmol) in ethanol (3 mL) was heated at 80° C. for 2 hours. After cooling, the reaction mixture was diluted with ethyl acetate, washed with a 2N aqueous solution of sodium hydroxide and brine, dried over sodium sulphate and the solvent evaporated. The crude product was purified by flash chromatography (dichloromethane to 9:1 dichloromethane/methanol) to yield 15 mg (27%) of the title product as a solid.

LRMS (m/z): 395 (M+1)⁺.

¹H NMR [symbol] (CDCl₃): 1.85-1.97 (m, 4H), 2.06-2.22 (m, 2H), 3.62-3.78 (m, 2H), 3.83 (m, 1H), 4.27 (d, 1H), 4.38 (d, 1H), 5.13-5.40 (m, 2H), 7.21 (dt, 1H), 7.66 (td, 1H), 7.91 (s, 1H), 8.35 (s, 1H), 8.46 (s, 1H), 9.88 (dd, 1H).

### EXAMPLE 157

### (R)-3-(3-(5-Cyclopropyl-2-(6-fluoroimidazo[1,2-a]pyridin-3-yl)pyrimidin-4-ylamino) piperidin-1-yl)-3-oxopropanenitrile

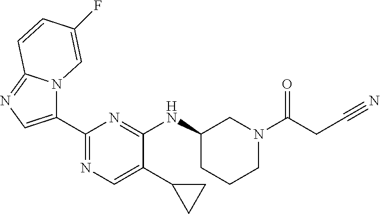 [SMILES: N#CCC(=O)N1CCC[C@@H](Nc2nc(-c3cnc4ccc(F)cn34)ncc2C2CC2)C1]

Obtained as a white solid (69%) from (R)-5-cyclopropyl-2-(6-fluoroimidazo[1,2-a]pyridin-3-yl)-N-(piperidin-3-yl)pyrimidin-4-amine (95 mg, 0.27 mmol, Preparation 64b) and 3-[(2,5-dioxopyrrolidin-1-yl)oxy]-3-oxopropanenitrile (59 mg, 0.32 mmol, prepared as described in BE875054(A1)) following the experimental procedure as described in Example 46 followed by purification by flash chromatography (dichloromethane to 9:1 dichloromethane/methanol).

LRMS (m/z): 420 (M+1)⁺.

¹H NMR [symbol] (CDCl₃): 0.61 (dd, 2H), 0.94-1.07 (m, 2H), 1.53 (td, 1H), 1.72-2.06 (m, 3H), 2.07-2.18 (m, 2H), 3.70 (t, 1H), 3.81 (dd, 1H), 3.98 (dd, 1H), 4.36 (ddd, 1H), 5.21-5.57 (m, 2H), 7.13-7.28 (m, 2H), 7.67 (td, 1H), 8.13 (s, 1H), 8.48 (s, 1H), 9.95 (ddd, 1H).

### EXAMPLE 158

### (R)-4-(1-(2-Cyanoacetyl)piperidin-3-ylamino)-2-(6-fluoroimidazo[1,2-a]pyridin-3-yl)pyrimidine-5-carbonitrile

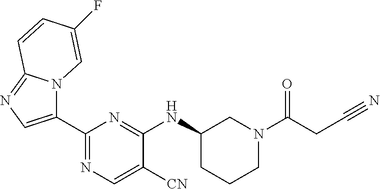 [SMILES: N#CCC(=O)N1CCC[C@@H](Nc2nc(-c3cnc4ccc(F)cn34)ncc2C#N)C1]

Obtained as a white solid (22%) from (R)-2-(6-fluoroimidazo[1,2-a]pyridin-3-yl)-4-(piperidin-3-ylamino)pyrimidine-5-carbonitrile (30 mg, 0.09 mmol, Preparation 66b) and 3-[(2,5-dioxopyrrolidin-1-yl)oxy]-3-oxopropanenitrile (20 mg, 0.11 mmol, prepared as described in BE875054(A1)) following the experimental procedure as described in Example 46 followed by purification by flash chromatography (1:1 hexane/ethyl acetate to ethyl acetate).

LRMS (m/z): 405 (M+1)⁺.

¹H NMR [symbol] (CDCl₃): 1.79-2.01 (m, 2H), 2.25 (d, 2H), 3.04 (dd, 1H), 3.14-3.46 (m, 1H), 3.59 (s, 2H), 3.65-3.97 (m, 1H), 4.20-4.47 (m, 1H), 4.66 (dd, 1H), 5.61-5.88 (m, 1H), 7.32 (td, 1H), 7.72 (dd, 1H), 8.53 (s, 1H), 8.57 (d, 1H), 8.71 (s, 1H), 9.75 (dd, 1H).

### EXAMPLE 159

### (R)-3-(3-(2-(6-Fluoroimidazo[1,2-a]pyridin-3-yl)-5-(methylsulfonyl)pyrimidin-4-ylamino)piperidin-1-yl)-3-oxopropanenitrile

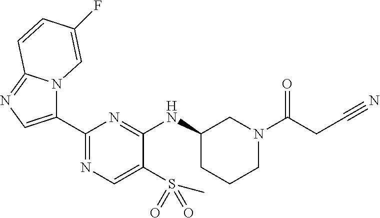 [SMILES: CS(=O)(=O)c1cnc(-c2cnc3ccc(F)cn23)nc1N[C@@H]1CCCN(C(=O)CC#N)C1]

Obtained as a white solid (34%) from (R)-2-(6-fluoroimidazo[1,2-a]pyridin-3-yl)-5-(methylsulfonyl)-N-(piperidin-3-yl)pyrimidin-4-amine (30 mg, 0.08 mmol, Preparation 68b) and 3-[(2,5-dioxopyrrolidin-1-yl)oxy]-3-oxopropanenitrile (17 mg, 0.09 mmol, prepared as described in BE875054(A1)) following the experimental procedure as described in Example 46.

LRMS (m/z): 458 (M+1)⁺.

¹H NMR [symbol] (CDCl₃): 0.77-2.20 (m, 5H), 3.15 (s, 3H), 3.45-3.67 (m, 4H), 3.75-4.02 (m, 2H), 4.49 (bs, 1H), 7.30-7.43 (m, 1H), 7.75 (d, 1H), 8.53-8.86 (m, 2H), 9.76-9.98 (m, 1H).

### EXAMPLE 160

### (R)-ethyl 4-(1-(2-Cyanoacetyl)piperidin-3-ylamino)-2-(6-fluoroimidazo[1,2-a]pyridin-3-yl)pyrimidine-5-carboxylate

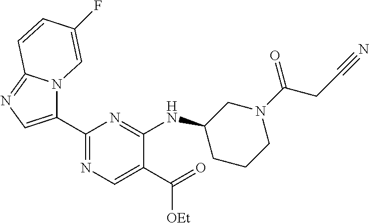 [SMILES: CCOC(=O)c1cnc(-c2cnc3ccc(F)cn23)nc1N[C@@H]1CCCN(C(=O)CC#N)C1]

Obtained as a white solid (96%) from (R)-ethyl 2-(6-fluoroimidazo[1,2-a]pyridin-3-yl)-4-(piperidin-3-ylamino)pyrimidine-5-carboxylate (80 mg, 0.21 mmol, Preparation 69b) and 3-[(2,5-dioxopyrrolidin-1-yl)oxy]-3-oxopropanenitrile (46 mg, 0.25 mmol, prepared as described in BE875054(A1)) following the experimental procedure as described in Example 46 followed by trituration in diethyl ether.

LRMS (m/z): 452 (M+1)⁺.

¹H NMR [symbol] (CDCl₃): 1.42 (t, 3H), 1.74-2.30 (m, 4H), 3.26 (dd, 1H), 3.41 (s, 2H), 3.53-3.83 (m, 4H), 4.38 (q, 2H), 7.28-7.36 (m, 1H), 7.72 (td, 1H), 8.34-8.54 (m, 1H), 8.63 (s, 1H), 8.96 (s, 1H), 9.88 (dd, 1H).

### EXAMPLE 161

### (R)-4-(1-(2-Cyanoacetyl)piperidin-3-ylamino)-2-(6-fluoroimidazo[1,2-a]pyridin-3-yl)pyrimidine-5-carboxylic acid

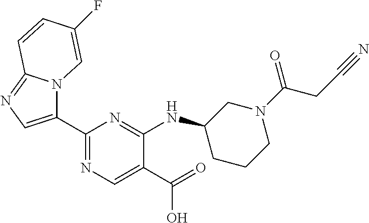 [SMILES: N#CCC(=O)N1CCC[C@@H](Nc2nc(-c3cnc4ccc(F)cn34)ncc2C(=O)O)C1]

A solution of lithium hydroxide (47 mg, 1.12 mmol) in water (2 mL) was added to a solution of (R)-ethyl 4-(1-(2-cyanoacetyl)piperidin-3-ylamino)-2-(6-fluoroimidazo[1,2-a]pyridin-3-yl)pyrimidine-5-carboxylate (50 mg, 0.11 mmol, Example 160) in a mixture of methanol (10 mL) and tetrahydrofuran (7 mL) and the resulting mixture was stirred at room temperature for 1 hour. The solvents were evaporated and the residue was treated with water and acidified to pH=4 by addition of 2N aqueous hydrochloric acid solution. The solid formed was filtered, washed with water and dried to yield 15 mg (32%) of the title compound.

LRMS (m/z): 424 (M+1)⁺.

¹H NMR δ (DMSO-d₆): 1.77 (bs, 3H), 2.10 (bs, 1H), 3.09 (dd, 1H), 3.21 (d, 1H), 3.55 (bs, 1H), 3.68-3.89 (m, 1H), 4.10 (s, 2H), 4.39 (d, 1H), 7.63 (ddd, 1H), 7.89 (ddd, 1H), 8.41-8.60 (m, 1H), 8.66 (s, 1H), 8.87 (d, 1H), 9.90 (ddd, 1H), 13.47 (s, 1H).

### EXAMPLE 162

### (R)-4-(1-(2-Cyanoacetyl)piperidin-3-ylamino)-2-(6-fluoroimidazo[1,2-a]pyridin-3-yl)pyrimidine-5-carboxamide

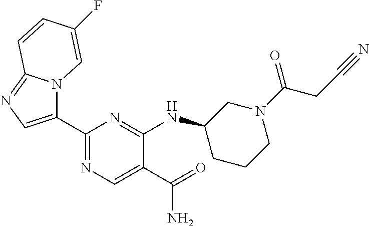 [SMILES: N#CCC(=O)N1CCC[C@@H](Nc2nc(-c3cnc4ccc(F)cn34)ncc2C(N)=O)C1]

1-Hydroxypyrrolidine-2,5-dione (25 mg, 0.22 mmol) and N,N′-methanediylidenedi propan-2-amine (33 μL, 0.21 mmol) were added to a solution of (R)-4-(1-(2-cyano acetyl)piperidin-3-ylamino)-2-(6-fluoroimidazo[1,2-a]pyridin-3-yl)pyrimidine-5-carboxylic acid (100 mg, 0.20 mmol, Example 161) in N,N-dimethylformamide (1 mL) at 0° C. The resulting mixture was stirred at room temperature overnight. Water (7 mL) was then added and the solid that precipitated was filtered, washed with water and dried. The solid was redissolved in dioxane (1 mL) and a solution of aqueous ammonium hydroxide (30 μL) was added. After stirring for 5 hours at room temperature, water was added and the resulting solution was acidified by addition of 2N aqueous hydrochloric acid solution. The suspension formed was extracted with methylene chloride and the organic layer was separated, washed with a 4% aqueous solution of sodium carbonate and brine, dried over sodium sulphate and the solvent evaporated. The crude product was purified by reverse phase chromatography (C-18 silica from Waters, water/1:1 acetonitrile-methanol as eluents [0.1% v/v formic acid buffered] 0% to 100%) to yield 3 mg (5%) of the title compound as a solid.

LRMS (m/z): 423 (M+1)⁺.

¹H NMR δ (DMSO-d₆): 1.70 (ms, 8H), 2.09 (m, 4H), 2.99 (dd, 1H), 3.20 (m, 4H), 3.60 (m, 1H), 3.73 (m, 1H), 3.81 (m, 1H), 3.86-4.02 (m, 1H), 4.04 (d, 1H), 4.10 (d, 1H), 4.31 (m, 1H), 4.43 (dd, 1H), 7.60 (dd, 2H), 7.78-7.93 (m, 2H), 8.19 (bs, 2H), 8.62 (d, 2H), 8.85 (d, 2H), 9.24 (d, 1H), 9.33 (d, 1H), 9.81-9.92 (m, 1H).

### EXAMPLE 163

### (R)-3-(3-(2-(6-Fluoroimidazo[1,2-a]pyridin-3-yl)-5-methoxypyrimidin-4-ylamino) piperidin-1-yl)-3-oxopropanenitrile

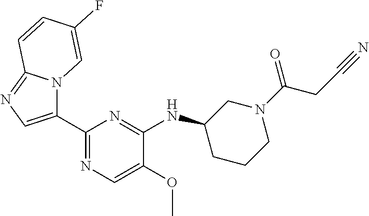 [SMILES: COc1cnc(-c2cnc3ccc(F)cn23)nc1N[C@@H]1CCCN(C(=O)CC#N)C1]

Obtained as a white solid (80%) from (R)-2-(6-fluoroimidazo[1,2-a]pyridin-3-yl)-5-methoxy-N-(piperidin-3-yl)pyrimidin-4-amine (210 mg, 0.61 mmol, Preparation 71b) and 3-[(2,5-dioxopyrrolidin-1-yl)oxy]-3-oxopropanenitrile (134 mg, 0.74 mmol, prepared as described in BE875054(A1)) following the experimental procedure as described in Example 46.

LRMS (m/z): 410 (M+1)⁺.

¹H NMR [symbol] (CDCl₃): 1.87 (d, 2H), 2.15 (m, 1H), 3.38 (d, 2H), 3.60 (m, 2H), 3.95 (s, 3H), 4.14-4.42 (m, 2H), 5.21-5.43 (m, 2H), 7.14-7.23 (m, 1H), 7.57-7.75 (m, 1H), 7.79-7.93 (m, 1H), 8.36 (s, 1H), 8.47 (s, 1H), 9.69-10.09 (m, 1H).

### EXAMPLE 164

### (R)-3-(3-(2-(6-Fluoroimidazo[1,2-a]pyridin-3-yl)-6-(M−1,2,4-triazol-1-yl)pyrimidin-4-ylamino)piperidin-1-yl)-3-oxopropanenitrile

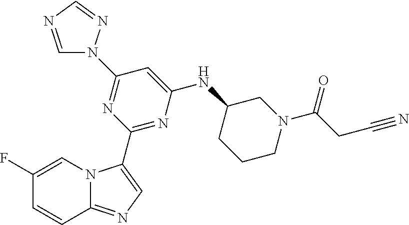 [SMILES: N#CCC(=O)N1CCC[C@@H](Nc2cc(-n3cncn3)nc(-c3cnc4ccc(F)cn34)n2)C1]

Obtained as a white solid (45%) from (R)-2-(6-fluoroimidazo[1,2-a]pyridin-3-yl)-N-(piperidin-3-yl)-6-(1H-1,2,4-triazol-1-yl)pyrimidin-4-amine (61 mg, 0.16 mmol, Preparation 74b) and 3-[(2,5-dioxopyrrolidin-1-yl)oxy]-3-oxopropanenitrile (44 mg, 0.24 mmol, prepared as described in BE875054(A1)) following the experimental procedure as described in Example 46 followed by purification by flash chromatography (dichloromethane to 9:1 dichloromethane/methanol).

LRMS (m/z): 447 (M+1)⁺.

¹H NMR [symbol] (CDCl₃): 0.87 (m, 1H), 1.18-1.41 (m, 3H), 1.74-2.08 (m, 2H), 2.18 (m, 1H), 3.30-3.69 (m, 2H), 3.83 (m, 1H), 4.32 (d, 1H), 5.25-5.53 (m, 1H), 6.71-6.84 (m, 1H), 7.20-7.38 (m, 1H), 7.66-7.84 (m, 1H), 8.13 (bs, 1H), 8.51-8.69 (m, 1H), 9.22 (s, 1H), 9.74 (d, 1H).

### EXAMPLE 165

### (R)-3-(3-(2-(6-Fluoroimidazo[1,2-a]pyridin-3-yl)-6-(4-methylpiperazin-1-yl)pyrimidin-4-ylamino)piperidin-1-yl)-3-oxopropanenitrile

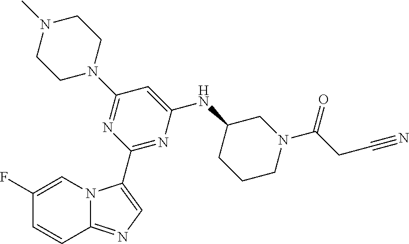 [SMILES: CN1CCN(c2cc(N[C@@H]3CCCN(C(=O)CC#N)C3)nc(-c3cnc4ccc(F)cn34)n2)CC1]

Obtained as a white solid (75%) from (R)-2-(6-fluoroimidazo[1,2-a]pyridin-3-yl)-6-(4-methylpiperazin-1-yl)-N-(piperidin-3-yl)pyrimidin-4-amine (55 mg, 0.13 mmol, Preparation 75b) and 3-[(2,5-dioxopyrrolidin-1-yl)oxy]-3-oxopropanenitrile (37 mg, 0.20 mmol, prepared as described in BE875054(A1)) following the experimental procedure as described in Example 46 followed by purification by flash chromatography (dichloromethane to 8:2 dichloromethane/methanol).

LRMS (m/z): 478 (M+1)⁺.

¹H NMR [symbol] (CDCl₃): 1.53-1.89 (m, 5H), 1.96 (m, 1H), 2.15 (ddd, 1H), 2.37 (s, 3H), 2.54 (t, 4H), 2.97 (d, 1H), 3.28-3.37 (m, 1H), 3.43-3.52 (m, 1H), 3.64-3.74 (m, 4H), 4.08 (d, 1H), 4.50-4.73 (m, 1H), 5.48 (d, 1H), 7.21 (dddd, 1H), 7.66 (td, 1H), 8.45 (d, 1H), 9.81 (ddd, 1H).

### EXAMPLE 166

### (R)-3-(3-(2-(6-Fluoroimidazo[1,2-a]pyridin-3-yl)-6-(piperazin-1-yl)pyrimidin-4-ylamino)piperidin-1-yl)-3-oxopropanenitrile

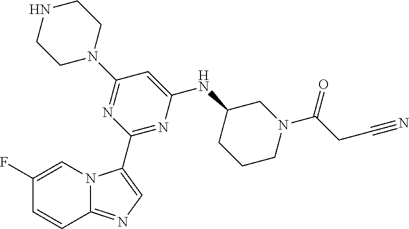 [SMILES: N#CCC(=O)N1CCC[C@@H](Nc2cc(N3CCNCC3)nc(-c3cnc4ccc(F)cn34)n2)C1]

Palladium on carbon (10%, 36 mg) was added to a solution of (R)-benzyl 446-(1-(2-cyanoacetyl)piperidin-3-ylamino)-2-(6-fluoroimidazo[1,2-a]pyridin-3-yl)pyrimidin-4-yl)piperazine-1-carboxylate (204 mg, 0.34 mmol, Preparation 76c) in methanol (20 mL) and the resulting mixture was stirred under hydrogen pressure (30 psi) for 3 days. The catalyst was then filtered and the solvent removed under vacuum evaporation. The crude product was purified by flash chromatography (dichloromethane to 8:2 dichloromethane/methanol) to obtain the title compound as a white solid (41%).

LRMS (m/z): 464 (M+1)⁺.

¹H-NMR δ (DMSO-d₆): 1.3-2.1 (m, 4H), 2.6-3.2 (m, 5H), 3.5 (m, 4H), 3.9-4.1 (m, 2H), 4.4 (d, 1H), 5.6 (m, 1H), 7.0 (t, 1H), 7.5 (m, 1H), 7.8 (m, 1H), 8.4 (d, 1H), 9.9 (s, 1H).

### EXAMPLE 167

### (R)-3-(3-(2-(6-Fluoroimidazo[1,2-a]pyridin-3-yl)-6-(4-(methylsulfonyl)piperazin-1-yl)pyrimidin-4-ylamino)piperidin-1-yl)-3-oxopropanenitrile

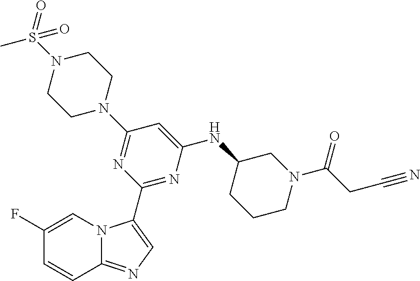 [SMILES: CS(=O)(=O)N1CCN(c2cc(N[C@@H]3CCCN(C(=O)CC#N)C3)nc(-c3cnc4ccc(F)cn34)n2)CC1]

Triethylamine (20 μL, 0.14 mmol) and methanesulfonyl chloride (9 μL, 0.12 mmol) were added to a solution of (R)-3-(3-(2-(6-fluoroimidazo[1,2-a]pyridin-3-yl)-6-(piperazin-1-yl)pyrimidin-4-ylamino)piperidin-1-yl)-3-oxopropanenitrile (42 mg, 0.09 mmol, Example 166) in dichloromethane (2 mL) and the resulting mixture was stirred at room temperature overnight. The reaction mixture was then diluted with dichloromethane and the organic solution was washed with water and brine, dried over magnesium sulphate and the solvent evaporated. The crude product was purified by flash chromatography (dichloromethane to 9:1 dichloromethane/methanol) to give the title compound as a white solid (53%).

LRMS (m/z): 542 (M+1)⁺.

¹H-NMR δ (CDCl₃): 1.7-2.2 (m, 4H), 2.8 (d, 2H), 3.0 (bs, 1H), 3.3-3.9 (m, 8H), 4.1-4.8 (m, 2H), 5.5 (d, 1H), 7.2 (m, 1H), 7.7 (m, 1H), 8.4 (d, 1H), 9.8 (m, 1H).

### EXAMPLE 168

### (R)-3-(3-(2-(6-Fluoroimidazo[1,2-a]pyridin-3-yl)-6-morpholinopyrimidin-4-ylamino) piperidin-1-yl)-3-oxopropanenitrile

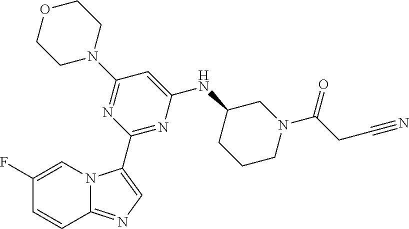 [SMILES: N#CCC(=O)N1CCC[C@@H](Nc2cc(N3CCOCC3)nc(-c3cnc4ccc(F)cn34)n2)C1]

Obtained as a white solid (44%) from (R)-2-(6-fluoroimidazo[1,2-a]pyridin-3-yl)-6-morpholino-N-(piperidin-3-yl)pyrimidin-4-amine (68 mg, 0.17 mmol, Preparation 77b) and 3-[(2,5-dioxopyrrolidin-1-yl)oxy]-3-oxopropanenitrile (47 mg, 0.26 mmol, prepared as described in BE875054(A1)) following the experimental procedure as described in Example 46 followed by purification by flash chromatography (dichloromethane to 9:1 dichloromethane/methanol).

LRMS (m/z): 465 (M+1)⁺.

¹H NMR [symbol] (CDCl₃): 1.58-1.90 (m, 2H), 1.91-2.03 (m, 1H), 2.15 (dd, 1H), 2.88-3.04 (m, 1H), 3.17 (d, 1H), 3.26-3.42 (m, 2H), 3.43-3.73 (m, 5H), 3.78-3.89 (m, 4H), 4.03-4.18 (m, 1H), 4.39-4.60 (m, 1H), 4.60-4.83 (m, 1H), 5.48 (d, 1H), 7.21 (dq, 1H), 7.66 (ddd, 1H), 8.45 (d, 1H), 9.80 (ddd, 1H).

### EXAMPLE 169

### (R)-3-(3-(6-(Dimethylamino)-2-(6-fluoroimidazo[1,2-a]pyridin-3-yl)pyrimidin-4-ylamino)piperidin-1-yl)-3-oxopropanenitrile

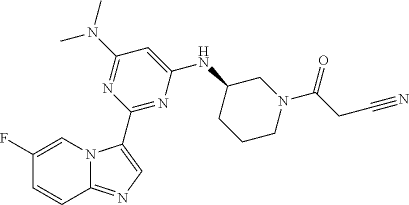 [SMILES: CN(C)c1cc(N[C@@H]2CCCN(C(=O)CC#N)C2)nc(-c2cnc3ccc(F)cn23)n1]

Obtained as a white solid (75%) from (R)-2-(6-fluoroimidazo[1,2-a]pyridin-3-yl)-N⁴,N⁴-dimethyl-N⁶-(piperidin-3-yl)pyrimidine-4,6-diamine (46 mg, 0.13 mmol, Preparation 78b) and 3-[(2,5-dioxopyrrolidin-1-yl)oxy]-3-oxopropanenitrile (35 mg, 0.19 mmol, prepared as described in BE875054(A1)) following the experimental procedure as described in Example 46 followed by purification by flash chromatography (dichloromethane to 9:1 dichloromethane/methanol).

LRMS (m/z): 423 (M+1)⁺.

¹H NMR [symbol] (CDCl₃): 1.58 (s, 6H), 1.64-1.89 (m, 2H), 1.91-2.02 (m, 1H), 2.06-2.25 (m, 2H), 2.85-2.97 (m, 1H), 3.26-3.43 (m, 1H), 3.48-3.84 (m, 2H), 4.10 (bs, 1H), 4.39-4.71 (m, 1H), 5.26-5.37 (m, 1H), 5.39 (s, 1H), 7.20 (dddd, 1H), 7.66 (td, 1H), 8.46 (d, 1H), 9.93 (ddd, 1H).

### EXAMPLE 170

### (R)-3-(3-(2-(6-Fluoroimidazo[1,2-a]pyridin-3-yl)-6-(2-morpholinoethylamino) pyrimidin-4-ylamino)piperidin-1-yl)-3-oxopropanenitrile

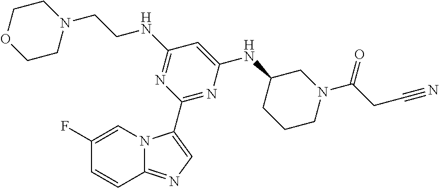 [SMILES: N#CCC(=O)N1CCC[C@@H](Nc2cc(NCCN3CCOCC3)nc(-c3cnc4ccc(F)cn34)n2)C1]

Obtained as a yellowish solid (77%) from (R)-2-(6-fluoroimidazo[1,2-a]pyridin-3-yl)-N⁴-(2-morpholinoethyl)-N⁶-(piperidin-3-yl)pyrimidine-4,6-diamine (45 mg, 0.10 mmol, Preparation 79b) and 3-[(2,5-dioxopyrrolidin-1-yl)oxy]-3-oxopropanenitrile (28 mg, 0.15 mmol, prepared as described in BE875054(A1)) following the experimental procedure as described in Example 46 followed by purification by flash chromatography (dichloromethane to 88:12 dichloromethane/methanol).

LRMS (m/z): 508 (M+1)⁺.

¹H NMR [symbol] (CDCl₃): 1.55-2.05 (m, 4H), 2.10-2.25 (m, 1H), 2.46-2.60 (m, 4H), 2.67 (t, 1H), 2.81-3.04 (m, 1H), 3.31 (ddd, 1H), 3.36-3.50 (m, 2H), 3.55 (s, 2H), 3.77 (d, 6H), 4.41-4.80 (m, 2H), 5.26-5.35 (m, 1H), 5.38 (s, 1H), 7.12-7.25 (m, 1H), 7.66 (td, 1H), 8.46 (d, 1H), 9.92 (d, 1H).

### EXAMPLE 171

### (R)-3-(3-(2-(6-Fluoroimidazo[1,2-a]pyridin-3-yl)-6-(2-methoxyethylamino)pyrimidin-4-ylamino)piperidin-1-yl)-3-oxopropanenitrile

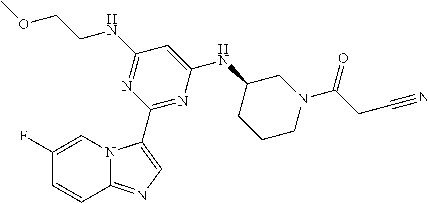 [SMILES: COCCNc1cc(N[C@@H]2CCCN(C(=O)CC#N)C2)nc(-c2cnc3ccc(F)cn23)n1]

Obtained as a white solid (68%) from (R)-2-(6-fluoroimidazo[1,2-a]pyridin-3-yl)-N⁴-(2-methoxyethyl)-N⁶-(piperidin-3-yl)pyrimidine-4,6-diamine (123 mg, 0.32 mmol, Preparation 80b) and 3-[(2,5-dioxopyrrolidin-1-yl)oxy]-3-oxopropanenitrile (87 mg, 0.48 mmol, prepared as described in BE875054(A1)) following the experimental procedure as described in Example 46 followed by purification by flash chromatography (dichloromethane to 9:1 dichloromethane/methanol).

LRMS (m/z): 453 (M+1)⁺.

¹H-NMR δ (DMSO-d₆): 1.4-2.0 (m, 4H), 2.5-3.1 (m, 2H), 3.3 (d, 2H), 3.3 (s, 3H), 3.4-3.7 (m, 2H), 3.9-4.1 (m, 2H), 4.5 (bs, 1H), 5.4 (m, 1H), 6.8 (dd, 1H), 6.9 (s, 1H), 7.5 (m, 1H), 7.8 (m, 1H), 8.4 (d, 1H), 10.1 (s, 1H).

### EXAMPLE 172

### (S)-1-(6-0R)-1-(2-Cyanoacetyl)piperidin-3-ylamino)-2-(6-fluoroimidazo[1,2-a]pyridin-3-yl)pyrimidin-4-yl)pyrrolidine-2-carboxylic acid

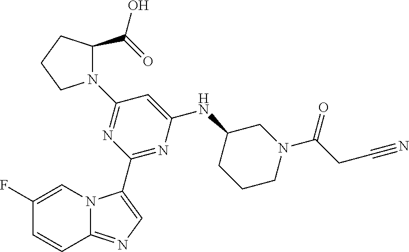 [SMILES: N#CCC(=O)N1CCC[C@@H](Nc2cc(N3CCC[C@H]3C(=O)O)nc(-c3cnc4ccc(F)cn34)n2)C1]

Obtained as a white solid (58%) from (S)-1-(2-(6-fluoroimidazo[1,2-a]pyridin-3-yl)-6-((R)-piperidin-3-ylamino)pyrimidin-4-yl)pyrrolidine-2-carboxylic acid (30 mg, 0.07 mmol, Preparation 82c) and 3-[(2,5-dioxopyrrolidin-1-yl)oxy]-3-oxopropanenitrile (19 mg, 0.10 mmol, prepared as described in BE875054(A1)) following the experimental procedure as described in Example 46 followed by purification by reverse phase chromatography (C-18 silica from Waters, water/1:1 acetonitrile-methanol as eluents [0.1% v/v formic acid buffered] 0% to 100%).

LRMS (m/z): 493 (M+1)⁺.

¹H NMR δ (DMSO-d₆): 1.41-1.65 (m, 2H), 1.70-1.84 (m, 1H), 1.94-2.11 (m, 4H), 2.20-2.37 (m, 2H), 2.89-3.81 (m, 8H), 4.06 (d, 1H), 4.50-4.68 (m, 1H), 5.29-5.56 (m, 1H), 6.87-7.00 (m, 1H), 7.48 (m, 1H), 7.75 (m, 1H), 8.35-8.45 (m, 1H), 9.94 (bs, 1H).

### EXAMPLE 173

### (R)-3-(3-(2-(6-Fluoroimidazo[1,2-a]pyridin-3-yl)-6-methoxypyrimidin-4-ylamino) piperidin-1-yl)-3-oxopropanenitrile

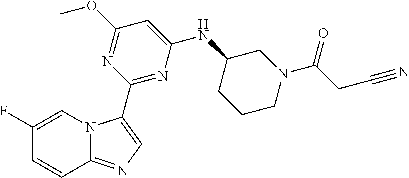 [SMILES: COc1cc(N[C@@H]2CCCN(C(=O)CC#N)C2)nc(-c2cnc3ccc(F)cn23)n1]

Obtained as a white solid (48%) from (R)-2-(6-fluoroimidazo[1,2-a]pyridin-3-yl)-6-methoxy-N-(piperidin-3-yl)pyrimidin-4-amine (90 mg, 0.26 mmol, Preparation 83b) and 3-[(2,5-dioxopyrrolidin-1-yl)oxy]-3-oxopropanenitrile (72 mg, 0.40 mmol, prepared as described in BE875054(A1)) following the experimental procedure as described in Example 46 followed by purifications by flash chromatography (dichloromethane to 95:5 dichloromethane/methanol) and reverse phase chromatography (C-18 silica from Waters, water/1:1 acetonitrile-methanol as eluents [0.1% v/v formic acid buffered] 0% to 100%).

LRMS (m/z): 410 (M+1)⁺.

¹H NMR [symbol] (CDCl₃): 1.67-2.02 (m, 6H), 2.07-2.24 (m, 2H), 3.10 (dd, 2H), 3.29-3.50 (m, 4H), 3.52-3.67 (m, 4H), 3.69-3.79 (m, 2H), 3.86 (d, 1H), 4.03 (s, 3H), 4.06 (s, 3H), 4.42 (d, 1H), 4.82 (dd, 2H), 5.64 (d, 2H), 7.17-7.33 (m, 2H), 7.69 (td, 2H), 8.52 (d, 2H), 9.77-9.95 (m, 2H).

### EXAMPLE 174

### (R)-3-(3-(2-(6-Fluoroimidazo[1,2-a]pyridin-3-yl)-6-(2-(pyrrolidin-1-yl)ethoxy)pyrimidin-4-ylamino)piperidin-1-yl)-3-oxopropanenitrile

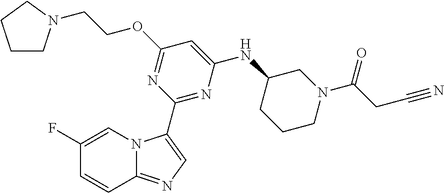 [SMILES: N#CCC(=O)N1CCC[C@@H](Nc2cc(OCCN3CCCC3)nc(-c3cnc4ccc(F)cn34)n2)C1]

Obtained as a white solid (58%) from (R)-2-(6-fluoroimidazo[1,2-a]pyridin-3-yl)-N-(piperidin-3-yl)-6-(2-(pyrrolidin-1-yl)ethoxy)pyrimidin-4-amine (111 mg, 0.26 mmol, Preparation 84c) and 3-[(2,5-dioxopyrrolidin-1-yl)oxy]-3-oxopropanenitrile (71 mg, 0.40 mmol, prepared as described in BE875054(A1)) following the experimental procedure as described in Example 46 followed by purification by flash chromatography (dichloromethane to 3:1 dichloromethane/methanol).

LRMS (m/z): 493 (M+1)⁺.

¹H NMR [symbol] (CDCl₃): 1.77 (d, 6H), 1.95-2.32 (m, 2H), 2.56-2.83 (m, 4H), 2.98 (t, 2H), 3.12-3.50 (m, 2H), 3.50-3.80 (m, 2H), 3.80-4.11 (m, 1H), 4.30 (bs, 1H), 4.49-4.67 (m, 2H), 4.98 (d, 1H), 5.12 (bs, 1H), 5.69 (s, 1H), 7.14-7.30 (m, 1H), 7.68 (dt, 1H), 8.49 (s, 1H), 9.65-9.92 (m, 1H).

### EXAMPLE 175

### (R)-3-(3-(2-(6-Fluoroimidazo[1,2-a]pyridin-3-yl)-6-(2-morpholinoethoxy)pyrimidin-4-ylamino)piperidin-1-yl)-3-oxopropanenitrile

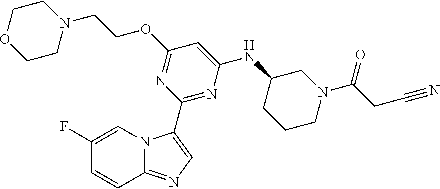 [SMILES: N#CCC(=O)N1CCC[C@@H](Nc2cc(OCCN3CCOCC3)nc(-c3cnc4ccc(F)cn34)n2)C1]

Obtained as a white solid (76%) from (R)-2-(6-fluoroimidazo[1,2-a]pyridin-3-yl)-6-(2-morpholinoethoxy)-N-(piperidin-3-yl)pyrimidin-4-amine (102 mg, 0.23 mmol, Preparation 85b) and 3-[(2,5-dioxopyrrolidin-1-yl)oxy]-3-oxopropanenitrile (63 mg, 0.35 mmol, prepared as described in BE875054(A1)) following the experimental procedure as described in Example 46 followed by purification by flash chromatography (dichloromethane to 88:12 dichloromethane/methanol).

LRMS (m/z): 509 (M+1)⁺.

¹H-NMR δ (CDCl₃): 1.7-2.2 (m, 4H), 2.6 (m, 4H), 2.8 (q, 2H), 3.3-3.6 (m, 4H), 3.7 (m, 4H), 3.8-4.0 (m, 1H), 4.4 (dd, 1H), 4.6 (q, 2H), 4.8 (d, 1H), 5.6 (d, 1H), 7.2 (m, 2H), 7.7 (m, 1H), 8.5 (d, 1H), 9.8 (m, 1H).

### EXAMPLE 176

### (R)-3-(3-(2-(6-Fluoroimidazo[1,2-a]pyridin-3-yl)-6-(2-methoxyethoxy)pyrimidin-4-ylamino)piperidin-1-yl)-3-oxopropanenitrile

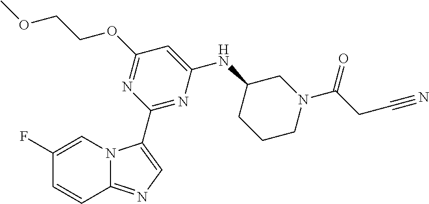 [SMILES: COCCOc1cc(N[C@@H]2CCCN(C(=O)CC#N)C2)nc(-c2cnc3ccc(F)cn23)n1]

Obtained as a white solid (62%) from (R)-2-(6-fluoroimidazo[1,2-a]pyridin-3-yl)-6-(2-methoxyethoxy)-N-(piperidin-3-yl)pyrimidin-4-amine (62 mg, 0.16 mmol, Preparation 86b) and 3-[(2,5-dioxopyrrolidin-1-yl)oxy]-3-oxopropanenitrile (44 mg, 0.24 mmol, prepared as described in BE875054(A1)) following the experimental procedure as described in Example 46 followed by purification by flash chromatography (dichloromethane to 9:1 dichloromethane/methanol).

LRMS (m/z): 454 (M+1)⁺.

¹H-NMR δ (CDCl₃): 1.7-2.2 (m, 4H), 3.1-4.1 (m, 11H), 4.3-4.6 (m, 2H), 4.9 (d, 1H), 5.7 (s, 1H), 7.2 (m, 1H), 7.7 (m, 1H), 8.5 (m, 1H), 9.8 (m, 1H).

### EXAMPLE 177

### (R)-6-(1-(2-Cyanoacetyl)piperidin-3-ylamino)-2-(6-fluoroimidazo[1,2-a]pyridin-3-yl)-pyrimidine-4-carbonitrile

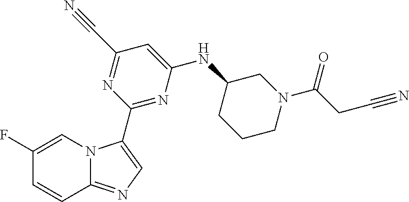 [SMILES: N#CCC(=O)N1CCC[C@@H](Nc2cc(C#N)nc(-c3cnc4ccc(F)cn34)n2)C1]

Obtained as a white solid (56%) from (R)-2-(6-fluoroimidazo[1,2-a]pyridin-3-yl)-6-(piperidin-3-ylamino)pyrimidine-4-carbonitrile (77 mg, 0.23 mmol, Preparation 87) and 3-[(2,5-dioxopyrrolidin-1-yl)oxy]-3-oxopropanenitrile (62 mg, 0.34 mmol, prepared as described in BE875054(A1)) following the experimental procedure as described in Example 46 followed by purification by flash chromatography (dichloromethane to 9:1 dichloromethane/methanol).

LRMS (m/z): 405 (M+1)⁺.

¹H NMR [symbol] (CDCl₃): 1.67-2.23 (m, 4H), 3.36-3.82 (m, 5H), 3.85-4.12 (m, 1H), 4.23-4.59 (m, 1H), 5.75 (d, 1H), 6.54-6.77 (m, 1H), 7.29 (d, 1H), 7.69 (dd, 1H), 8.42-8.69 (m, 1H), 9.76 (bs, 1H).

### EXAMPLE 178

### (R)-3-(3-(2-(6-Fluoroimidazo[1,2-a]pyridin-3-yl)-6-(2H-tetrazol-5-yl)pyrimidin-4-ylamino)piperidin-1-yl)-3-oxopropanenitrile

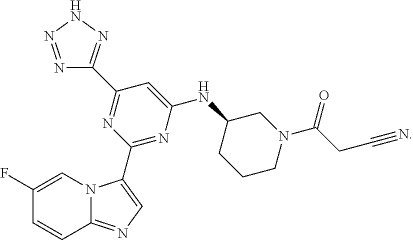 [SMILES: N#CCC(=O)N1CCC[C@@H](Nc2cc(-c3nn[nH]n3)nc(-c3cnc4ccc(F)cn34)n2)C1]

Obtained as a white solid (33%) from (R)-2-(6-fluoroimidazo[1,2-a]pyridin-3-yl)-N-(piperidin-3-yl)-6-(2H-tetrazol-5-yl)pyrimidin-4-amine (54 mg, 0.14 mmol, Preparation 88) and 3-[(2,5-dioxopyrrolidin-1-yl)oxy]-3-oxopropanenitrile (66 mg, 0.36 mmol, prepared as described in BE875054(A1)) following the experimental procedure as described in Example 46 followed by purification by reverse phase chromatography (C-18 silica from Waters, water/1:1 acetonitrile-methanol as eluents [0.1% v/v formic acid buffered] 0% to 100%).

LRMS (m/z): 448 (M+1)⁺.

¹H NMR δ (DMSO-d₆): 1.45-1.71 (m, 3H), 1.73-1.87 (m, 1H), 2.00-2.14 (m, 1H), 2.78-2.88 (m, 1H), 3.59 (d, 1H), 3.71 (d, 1H), 3.85-4.04 (m, 1H), 4.09 (d, 1H), 4.22-4.37 (m, 1H), 4.55 (d, 1H), 7.24 (d, 1H), 7.58 (td, 1H), 7.84 (dd, 1H), 7.99-8.11 (m, 1H), 8.66-8.78 (m, 1H), 9.92-10.07 (m, 1H).

### EXAMPLE 179

### (R)-3-(3-(2-(6-Fluoroimidazo[1,2-a]pyridin-3-yl)-6-(1-methyl-1H-tetrazol-5-yl)pyrimidin-4-ylamino)piperidin-1-yl)-3-oxopropanenitrile

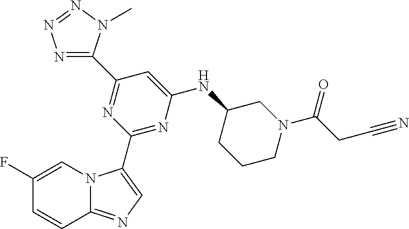 [SMILES: Cn1nnnc1-c1cc(N[C@@H]2CCCN(C(=O)CC#N)C2)nc(-c2cnc3ccc(F)cn23)n1]

Obtained as a white solid (45%) from (R)-2-(6-fluoroimidazo[1,2-a]pyridin-3-yl)-6-(1-methyl-1H-tetrazol-5-yl)-N-(piperidin-3-yl)pyrimidin-4-amine (48 mg, 0.12 mmol, Preparation 90) and 3-[(2,5-dioxopyrrolidin-1-yl)oxy]-3-oxopropanenitrile (33 mg, 0.18 mmol, prepared as described in BE875054(A1)) following the experimental procedure as described in Example 46. The crude product was triturated with chloroform and the insoluble solid was filtered, washed with hexane and dried.

LRMS (m/z): 462 (M+1)⁺.

¹H-NMR δ (CDCl₃): 1.6-2.2 (m, 4H), 2.5 (s, 3H), 3.3-3.7 (m, 4H), 4.4 (m, 1H), 4.6 (d, 2H), 7.3 (m, 3H), 7.7 (m, 1H), 8.5 (d, 1H), 9.8 (s, 1H).

### EXAMPLE 180

### (R)-3-(3-(2-(6-Fluoroimidazo[1,2-a]pyridin-3-yl)-6-(2-methyl-2H-tetrazol-5-yl)pyrimidin-4-ylamino)piperidin-1-yl)-3-oxopropanenitrile

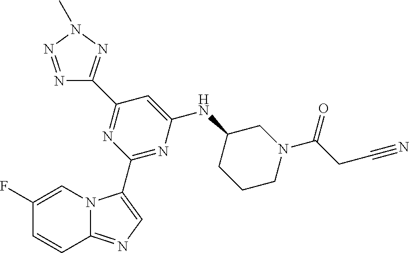 [SMILES: Cn1nnc(-c2cc(N[C@@H]3CCCN(C(=O)CC#N)C3)nc(-c3cnc4ccc(F)cn34)n2)n1]

Obtained as a white solid (66%) from (R)-2-(6-fluoroimidazo[1,2-a]pyridin-3-yl)-6-(2-methyl-2H-tetrazol-5-yl)-N-(piperidin-3-yl)pyrimidin-4-amine (40 mg, 0.10 mmol, Preparation 91) and 3-[(2,5-dioxopyrrolidin-1-yl)oxy]-3-oxopropanenitrile (28 mg, 0.15 mmol, prepared as described in BE875054(A1)) following the experimental procedure as described in Example 46.

LRMS (m/z): 462 (M+1)⁺.

¹H-NMR δ (CDCl₃): 1.6-2.2 (m, 4H), 2.4 (s, 3H), 3.4-3.7 (m, 4H), 4.3 (m, 1H), 4.5 (d, 2H), 7.3 (d, 3H), 7.7 (m, 1H), 8.6 (m, 1H), 10.2 (d, 1H).

### EXAMPLE 181

### (R)-6-(1-(2-Cyanoacetyl)piperidin-3-ylamino)-2-(6-fluoroimidazo[1,2-a]pyridin-3-yl)pyrimidine-4-carboxylic acid

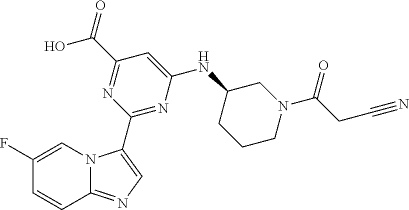 [SMILES: N#CCC(=O)N1CCC[C@@H](Nc2cc(C(=O)O)nc(-c3cnc4ccc(F)cn34)n2)C1]

Obtained as a white solid (35%) from (R)-2-(6-fluoroimidazo[1,2-a]pyridin-3-yl)-6-(piperidin-3-ylamino)pyrimidine-4-carboxylic acid (167 mg, 0.47 mmol, Preparation 92b) and 3-[(2,5-dioxopyrrolidin-1-yl)oxy]-3-oxopropanenitrile (128 mg, 0.70 mmol, prepared as described in BE875054(A1)) following the experimental procedure as described in Example 46 followed by purification by reverse phase chromatography (C-18 silica from Waters, water/1:1 acetonitrile-methanol as eluents [0.1% v/v formic acid buffered] 0% to 100%).

LRMS (m/z): 424 (M+1)⁺.

¹H-NMR δ (DMSO-d₆): 1.2-2.1 (m, 4H), 3.2 (m, 2H), 3.6-4.6 (m, 4H), 7.0 (d, 1H), 7.5 (m, 1H), 7.8 (m, 1H), 8.6 (d, 1H), 10.2 (s, 1H).

### EXAMPLE 182

### (R)-6-(1-(2-Cyanoacetyl)piperidin-3-ylamino)-2-(6-fluoroimidazo[1,2-a]pyridin-3-yl)pyrimidine-4-carboxamide

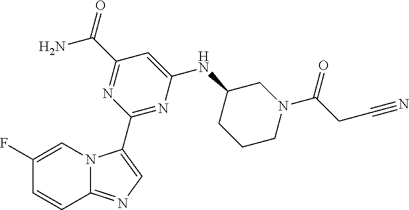 [SMILES: N#CCC(=O)N1CCC[C@@H](Nc2cc(C(N)=O)nc(-c3cnc4ccc(F)cn34)n2)C1]

1-Hydroxybenzotriazole hydrate (24 mg, 0.18 mmol), N-(3-dimethylaminopropyl)-N′-ethylcarbodiimide hydrochloride (34 mg, 0.18 mmol) and concentrated aqueous ammonium hydroxide solution (12 μL, 0.3 mmol) were added to a solution of (R)-6-(1-(2-Cyanoacetyl)piperidin-3-ylamino)-2-(6-fluoroimidazo[1,2-a]pyridin-3-yl)pyrimidine-4-carboxylic acid (50 mg, 0.12 mmol, Example 181) in N,N′-dimethylformamide (2 mL) and the resulting solution was stirred at room temperature overnight. The solvent was then evaporated and the residue was purified by reverse phase chromatography (C-18 silica from Waters, water/1:1 acetonitrile-methanol as eluents [0.1% v/v formic acid buffered] 0% to 100%) to yield 37 mg (74%) of the title compound as a white solid.

LRMS (m/z): 423 (M+1)⁺.

¹H-NMR δ (DMSO-d₆): 1.5-2.1 (m, 4H), 2.8-3.2 (m, 3H), 3.6-4.5 (m, 4H), 7.0 (m, 1H), 7.5 (m, 1H), 7.9 (m, 3H), 8.3 (s, 1H), 8.8 (d, 1H), 9.8 (m, 1H).

### EXAMPLE 183

### (R)-3-(3-(6-Amino-2-(6-fluoroimidazo[1,2-a]pyridin-3-yl)pyrimidin-4-ylamino) piperidin-1-yl)-3-oxopropanenitrile

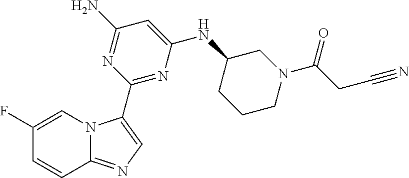 [SMILES: N#CCC(=O)N1CCC[C@@H](Nc2cc(N)nc(-c3cnc4ccc(F)cn34)n2)C1]

Obtained as a white solid (33%) from (R)-2-(6-fluoroimidazo[1,2-a]pyridin-3-yl)-N⁴-(piperidin-3-yl)pyrimidine-4,6-diamine (108 mg, 0.33 mmol, Preparation 93b) and 3-[(2,5-dioxopyrrolidin-1-yl)oxy]-3-oxopropanenitrile (90 mg, 0.49 mmol, prepared as described in BE875054(A1)) following the experimental procedure as described in Example 46 followed by purification by flash chromatography (dichloromethane to 85:15 dichloromethane/methanol).

LRMS (m/z): 395 (M+1)⁺.

¹H-NMR δ (DMSO-d₆): 1.4-2.0 (m, 4H), 2.5-3.2 (m, 2H), 3.5-4.1 (m, 4H), 4.5 (bs, 1H), 5.4 (d, 1H), 6.4 (s, 2H), 6.8 (dd, 1H), 7.5 (m, 1H), 7.7 (m, 1H), 8.3 (d, 1H), 10.1 (s, 1H).

### EXAMPLE 184

### (R)-3-(3-(5-Fluoro-2-(6-fluoroimidazo[1,2-a]pyridin-3-yl)-6-morpholinopyrimidin-4-ylamino)piperidin-1-yl)-3-oxopropanenitrile

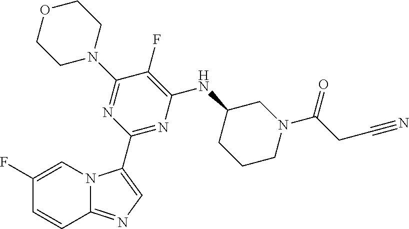 [SMILES: N#CCC(=O)N1CCC[C@@H](Nc2nc(-c3cnc4ccc(F)cn34)nc(N3CCOCC3)c2F)C1]

Obtained as a white solid (50%) from (R)-5-fluoro-2-(6-fluoroimidazo[1,2-a]pyridin-3-yl)-6-morpholino-N-(piperidin-3-yl)pyrimidin-4-amine (80 mg, 0.19 mmol, Preparation 95c) and 3-[(2,5-dioxopyrrolidin-1-yl)oxy]-3-oxopropanenitrile (53 mg, 0.29 mmol, prepared as described in BE875054(A1)) following the experimental procedure as described in Example 46 followed by purification by flash chromatography (dichloromethane to 95:5 dichloromethane/methanol).

LRMS (m/z): 483 (M+1)⁺.

¹H-NMR δ (CDCl₃): 1.7-2.2 (m, 4H), 3.2-3.9 (m, 12H), 4.2 (m, 1H), 4.8 (m, 1H), 7.2 (m, 1H), 7.7 (m, 1H), 8.4 (m, 1H), 9.7 (m, 1H).

### EXAMPLE 185

### (R)-1-((R)-3-(5-Fluoro-2-(6-fluoroimidazo[1,2-a]pyridin-3-yl)-6-morpholino pyrimidin-4-ylamino)piperidin-1-yl)-2,3-dihydroxypropan-1-one

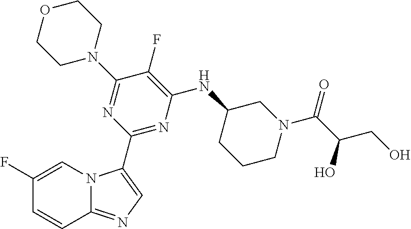 [SMILES: O=C([C@H](O)CO)N1CCC[C@@H](Nc2nc(-c3cnc4ccc(F)cn34)nc(N3CCOCC3)c2F)C1]

Obtained as a white solid (41%) from (R)-2,2-dimethyl-1,3-dioxolan-4-carboxylic acid (40 mg, 0.27 mmol) and (R)-5-fluoro-2-(6-fluoroimidazo[1,2-a]pyridin-3-yl)-6-morpholino —N-(piperidin-3-yl)pyrimidin-4-amine (73 mg, 0.18 mmol, Preparation 95c) following the experimental procedure as described in Example 95 followed by treatment with acetic acid (120° C., 1.5 hours). The crude product was purified by reverse phase chromatography (C-18 silica from Waters, water/1:1 acetonitrile-methanol as eluents [0.1% v/v formic acid buffered] 0% to 100%).

LRMS (m/z): 504 (M+1)⁺.

¹H-NMR δ (CDCl₃): 1.7-2.1 (m, 4H), 3.4-3.9 (m, 15H), 4.2-4.6 (m, 2H), 5.1 (m, 1H), 7.2 (m, 1H), 7.7 (dd, 1H), 8.4 (m, 1H), 9.7 (m, 1H).

### EXAMPLE 186

### (R)-3-(3-(2-(6-Fluoroimidazo[1,2-a]pyridin-3-yl)-6-methoxy-5-(trifluoromethyl)pyrimidin-4-ylamino)piperidin-1-yl)-3-oxopropanenitrile

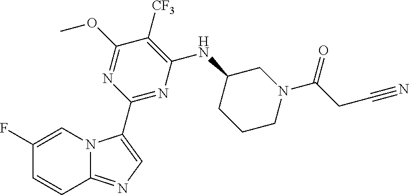 [SMILES: COc1nc(-c2cnc3ccc(F)cn23)nc(N[C@@H]2CCCN(C(=O)CC#N)C2)c1C(F)(F)F]

Obtained as a white solid (72%) from (R)-2-(6-fluoroimidazo[1,2-a]pyridin-3-yl)-6-methoxy-N-(piperidin-3-yl)-5-(trifluoromethyl)pyrimidin-4-amine (80 mg, 0.19 mmol, Preparation 97b) and 3-[(2,5-dioxopyrrolidin-1-yl)oxy]-3-oxopropanenitrile (53 mg, 0.29 mmol, prepared as described in BE875054(A1)) following the experimental procedure as described in Example 46 followed by purification by flash chromatography (dichloromethane to 95:5 dichloromethane/methanol).

LRMS (m/z): 478 (M+1)⁺.

¹H NMR [symbol] (CDCl₃): 1.72-1.89 (m, 2H), 1.91-2.03 (m, 1H), 2.13-2.27 (m, 1H), 3.14 (dd, 1H), 3.33-3.52 (m, 4H), 3.67 (d, 1H), 3.82 (dd, 1H), 4.12 (s, 2H), 4.30 (d, 1H), 4.52 (dd, 1H), 5.57 (bs, 1H), 7.30 (dd, 1H), 7.71 (dd, 1H), 8.64 (s, 1H), 9.60-9.80 (m, 1H).

### Pharmacological Activity

### In Vitro JAK Kinase Assays

Compounds were screened for their ability to inhibit JAK1, JAK2 and JAK3 using the assays as indicated below.

The catalytic domains of human JAK1 (aa 850-1154), JAK2 (aa 826-1132), JAK3 (aa 795-1124) and Tyk2 (aa 871-1187) were expressed as N-terminal GST-fusion proteins using a baculovirus expression system and were purchased from Carna Biosciences. The enzymatic activity was assayed using as substrate a biotinylated peptide, poly (GT)-Biotin (CisBio). The peptide concentration in the reactions was 60 nM for JAK1, 20 nM for JAK2, 140 nM for JAK3 and 50 nM for Tyk2. The degree of phosphorylation was detected by TR-FRET (time-resolved fluorescence energy transfer).

IC₅₀s of compounds were measured for each kinase in a reaction mixture containing the enzyme, ATP and the peptide in 8 mM MOPS (pH 7.0), 10 mM MgCl₂, 0.05% β-mercaptoethanol, 0.45 mg/mL BSA. The ATP concentration in the reactions was 3 μM for JAK1, 0.2 μM for JAK2, 0.6 μM for JAK3 and 1.8 μM for Tyk2. The enzymatic reactions took place for 30 minutes at room temperature. Then, the reactions were stopped with 20 μL of quench detection buffer (50 mM HEPES, 0.5 M KF, EDTA 0.25 M, 0.1% (w/v) BSA, pH 7.5) containing 0.115 μg/mL of anti-phosphoTyr (PT66)-Cryptate (CisBio) and a variable concentration of SA-XL665 (CisBio) to keep the SA-B ratio constant. Incubate for 3 h and read on Victor 2V spectrofluorometer (PerkinElmer) set to read fluorescence resonance energy transfer.

Some of the acronyms used above have the following meaning:

AA: aminoacids
GST: glutathione-S-transferase
MOPS: 3-(N-morpholino)propane sulfonic acid
BSA: bovine serum albumin
ATP: adenosine tri-phosphate
EDTA: ethylenediaminetetraacetic acid
HEPES: 4-(2-hydroxyethyl)-1-piperazineethanesulfonic acid

Table 1 depicts IC₅₀ values for certain exemplary compounds described in the invention. In Table 1, “A” represents an IC₅₀ value of less than 0.1 μM (100 nM), “B” represents an IC₅₀ value in the range of 0.1 μM (100 nM) to 1 μM (1000 nM), and C represents an IC₅₀ value higher than 1 μM (1000 nM).

TABLE 1

| | IC₅₀ JAK3 | IC₅₀ JAK2 | IC₅₀ JAK1 |
|---|---|---|---|
| Example No. | (μM) | (μM) | (μM) |
| 1 | A | A | B |
| 6 | B | B | C |
| 10 | B | A | C |
| 13 | B | B | C |
| 21 | A | A | B |
| 22 | A | A | C |
| 25 | B | B | B |
| 31 | B | A | B |
| 38 | B | A | C |
| 42 | C | B | C |
| 45 | B | B | C |
| 57 | A | A | C |
| 64 | A | A | A |
| 67 | B | A | B |
| 68 | A | A | B |
| 76 | B | A | C |
| 80 | A | A | A |
| 82 | C | B | C |
| 89 | C | B | C |
| 90 | A | A | C |
| 92 | C | A | B |
| 102 | B | A | C |
| 107 | C | A | C |
| 131 | A | A | A |
| 134 | B | A | B |
| 136 | A | A | A |
| 137 | B | A | C |
| 145 | B | A | B |
| 151 | A | A | B |
| 153 | A | A | C |
| 159 | B | A | C |
| 163 | A | A | B |
| 164 | A | A | B |
| 166 | A | A | B |
| 172 | B | A | B |
| 175 | A | A | A |
| 181 | B | A | B |
| 183 | A | A | A |
| 185 | B | A | B |

It can be seen from Table 1 that the compounds of formula (I) are potent inhibitors of JAK1, JAK2 and JAK3 kinases. Preferred imidazopyridine derivatives of the invention possess an IC₅₀ value for the inhibition of JAK1, JAK2 and JAK3 kinases (determined as defined above) of less than 1 μM, preferably less than 0.5 μM for each JAK kinase.

### Combinations

The imidazopyridine derivatives of the invention may also be combined with other active compounds in the treatment of a pathological condition or disease susceptible to amelioration by inhibition of Janus Kinases.

The combinations of the invention can optionally comprise one or more additional active substances which are known to be useful in the treatment of myeloproliferative disorders (such as polycythemia vera, essential thrombocythemia or mielofibrosis), leukemia, lymphoid malignancies and solid tumors; bone marrow and organ transplant rejection; immune-mediated diseases and inflammatory diseases, for example myeloproliferative disorders, leukemia, lymphoid malignancies and solid tumors; bone marrow and organ transplant rejection; and immune-mediated diseases; more in particular wherein the pathological condition or disease is selected from rheumatoid arthritis, multiple sclerosis, inflammatory bowel disease, dry eye, uveitis, allergic conjunctivitis, allergic rhinitis, asthma, chronic obstructive pulmonary disease (COPD), atopic dermatitis and psoriasis, such as (a) Dyhydrofolate reductase inhibitors, such as Methotrexate or CH-1504; (b) DHODH inhibitors such as leflunomide, teriflunomide, or the compounds described in the International Patent Application Nos. WO2008/077639 and WO2009021696; (c) Immunomodulators such as Glatiramer acetate (Copaxone), Laquinimod or Imiquimod; (d) Inhibitors of DNA synthesis and repair, such as Mitoxantrone or Cladribine; (e) Anti-alpha 4 integrin antibodies, such as Natalizumab (Tysabri); (f) Alpha 4 integrin antagonists such as R-1295, TBC-4746, CDP-323, ELND-002, Firategrast or TMC-2003; (g) Corticoids and glucocorticoids such as prednisone or methylprednisolone, fluticasone, mometasone, or beta-metasone; (h) Fumaric acid esters, such as BG-12; (i) Anti-TNF alpha antibodies, such as Infliximab, Adalimumab, or Certolizumab pegol; (j) Soluble TNF alpha receptors such as Ethanercept; (k) Anti-CD20 monoclonal antibodies such as Rituximab, Ocrelizumab Ofatumumab or TRU-015; (l) Anti-CD52 such as alemtuzumab; (m) Anti-CD25 such as daclizumab; (n) Anti-CD88, such as eculizumab or pexilizumab; (o) Anti-IL12R/IL23R, such as ustekinumab; (p) Calcineurin inhibitors such as cyclosporine A or tacrolimus; (q) IMPDH inhibitors, such as mycophenolate mophetyl; (r) Cannabinoid receptor agonists such as Sativex; (s) Chemokine CCR1 antagonists such as MLN-3897 or PS-031291; (t) Chemokine CCR2 antagonists such as INCB-8696; (u) NF-kappaB activation inhibitors such as MLN-0415; (v) S1P receptor agonists such as fingolimod, BAF-312, or ACT128800; (w) S1P liase inhibitors such as LX2931; (x) Syk inhibitors, such as R-112; (y) PKC inhibitors, such as NVP-AEB071; (z) M3 antagonist such as tiotropium or aclidinium; (aa) Long-acting beta adrenergic agonists such as salmeterol, formoterol or indacaterol; (bb) Vitamin D derivatives like calcipotriol (Daivonex); (cc) Phosphosdiesterase IV inhibitors such as roflumilast or GRC-4039; (dd) p38 Inhibitors such as ARRY-797; (ee) MEK inhibitors, such as ARRY-142886 or ARRY-438162; (ff) PI3Kδγ inhibitors; (gg) Interferons comprising Interferon beta 1a such as Avonex from Biogen Idec, CinnoVex from CinnaGen and Rebif from EMD Serono, and Interferon beta 1b such as Betaferon from Schering and Betaseron from Berlex; and (hh) Interferon alpha such as Sumiferon MP.

Specific examples of suitable corticoids and glucocorticoids that can be combined with the JAK inhibitors of the present invention are prednisolone, methylprednisolone, dexamethasone, dexamethasone cipecilate, naflocort, deflazacort, halopredone acetate, budesonide, beclomethasone dipropionate, hydrocortisone, triamcinolone acetonide, fluocinolone acetonide, fluocinonide, clocortolone pivalate, methylprednisolone aceponate, dexamethasone palmitoate, tipredane, hydrocortisone aceponate, prednicarbate, alclometasone dipropionate, halometasone, methylprednisolone suleptanate, mometasone furoate, rimexolone, prednisolone farnesylate, ciclesonide, butixocort propionate, RPR-106541, deprodone propionate, fluticasone propionate, fluticasone furoate, halobetasol propionate, loteprednol etabonate, betamethasone butyrate propionate, flunisolide, prednisone, dexamethasone sodium phosphate, triamcinolone, betamethasone 17-valerate, betamethasone, betamethasone dipropionate, hydrocortisone acetate, hydrocortisone sodium succinate, prednisolone sodium phosphate and hydrocortisone probutate.

Specific examples of suitable Syk kinase inhibitors that can be combined with the JAK inhibitors of the present invention are fosfamatinib (from Rigel), R-348 (from Rigel), R-343 (from Rigel), R-112 (from Rigel), piceatannol, 2-(2-Aminoethylamino)-4-[3-(trifluoromethyl)phenylamino]pyrimidine-5-carboxamide, R-091 (from Rigel), 6-[5-Fluoro-2-(3,4,5-trimethoxyphenylamino)pyrimidin-4-ylamino]-2,2-dimethyl-3,4-dihydro-2H-pyrido[3,2-b][1,4]oxazin-3-one benzenesulfonate (R-406 from Rigel), 1-(2,4,6-Trihydroxyphenyl)-2-(4-methoxyphenyl)ethan-1-one, N-[4-[6-(Cyclobutylamino)-9H-purin-2-ylamino]phenyl]-N-methylacetamide (QAB-205 from Novartis), 2-[7-(3,4-Dimethoxyphenyl)imidazo[1,2-c]pyrimidin-5-ylamino]pyridine-3-carboxamide dihydrochloride (BAY-61-3606 from Bayer) and AVE-0950 (from Sanofi-Aventis).

Specific examples of suitable M3 antagonists (anticholinergics) that can be combined with the JAK inhibitors of the present invention are tiotropium salts, oxitropium salts, flutropium salts, ipratropium salts, glycopyrronium salts, trospium salts, revatropate, espatropate, 3-[2-Hydroxy-2,2-bis(2-thienyl)acetoxy]-1-(3-phenoxypropyl)-1-azoniabicyclo[2.2.2]octane salts (in particular aclidinium salts, more preferably aclidinium bromide), 1-(2-Phenylethyl)-3-(9H-xanthen-9-ylcarbonyloxy)-1-azoniabicyclo[2.2.2]octane salts, 2-oxo-1,2,3,4-tetrahydroquinazoline-3-carboxylic acid endo-8-methyl-8-azabicyclo[3.2.1]oct-3-yl ester salts (DAU-5884), 3-(4-Benzylpiperazin-1-yl)-1-cyclobutyl-1-hydroxy-1-phenylpropan-2-one (NPC-14695), N-[1-(6-Aminopyridin-2-ylmethyl)piperidin-4-yl]-2(R)-[3,3-difluoro-1(R)-cyclopentyl]-2-hydroxy-2-phenylacetamide (J-104135), 2(R)-Cyclopentyl-2-hydroxy-N-[1-[4(S)-methylhexyl]piperidin-4-yl]-2-phenylacetamide (J-106366), 2(R)-Cyclopentyl-2-hydroxy-N-[1-(4-methyl-3-pentenyl)-4-piperidinyl]-2-phenylacetamide (J-104129), 1-[4-(2-Aminoethyl)piperidin-1-yl]-2(R)-[3,3-difluorocyclopent-[(R)-yl]-2-hydroxy-2-phenylethan-1-one (Banyu-280634), N—[N-[2-[N-[1-(Cyclohexylmethyl)piperidin-3(R)-ylmethyl]carbamoyl]ethyl]carbamoylmethyl]-3,3,3-triphenylpropionamide (Banyu CPTP), 2(R)-Cyclopentyl-2-hydroxy-2-phenylacetic acid 4-(3-azabicyclo[3.1.0]hex-3-yl)-2-butynyl ester (Ranbaxy 364057), UCB-101333, Merck's OrM3,7-endo-(2-hydroxy-2,2-diphenylacetoxy)-9,9-dimethyl-3-oxa-9-azoniatricyclo[3.3.1.0(2,4)]nonane salts, 7-(2,2-diphenylpropionyloxy)-7,9,9-trimethyl-3-oxa-9-azoniatricyclo[3.3.1.0*2,4]nonane salts, 7-hydroxy-7,9,9-trimethyl-3-oxa-9-azoniatricyclo[3.3.1.0*2,4]nonane 9-methyl-9H-fluorene-9-carboxylic acid ester salts, all of them optionally in the form of their racemates, their enantiomers, their diastereomers and mixtures thereof, and optionally in the form of their pharmacologically-compatible acid addition salts. Among the salts chlorides, bromides, iodides and methanesulphonates are preferred.

Specific examples of suitable long-acting beta adrenergic agonists (132-agonists) that can be combined with the JAK inhibitors of the present invention are terbutaline sulphate, eformoterol fumarate, formoterol fumarate, bambuterol, procaterol hydrochloride, sibenadet hydrochloride, mabuterol hydrochloride, albuterol sulphate, salbutamol sulphate, salmeterol xinafoate, carmoterol hydrochloride, (R)-albuterol hydrochloride, Levalbuterol hydrochloride; Levosalbutamol hydrochloride; (−)-Salbutamol hydrochloride, (R,R)-Formoterol tartrate; Arformoterol tartrate, Bedoradrine sulphate, Indacaterol, Trantinterol hydrochloride, AZD-3199, GSK-159802; GSK-597901, GSK-678007, GSK-642444; GSK-961081; AR-C98955AA, Milveterol hydrochloride, B1-1744-CL, and compounds described in the International Patent Applications Nos. WO2007/124898, WO2006/122788A1, WO2008/046598 and WO2008095720.

Specific examples of suitable Phosphosdiesterase IV inhibitors that can be combined with the JAK inhibitors of the present invention are benafentrine dimaleate, etazolate, denbufylline, rolipram, cipamfylline, zardaverine, arofylline, filaminast, tipelukast, tofimilast, piclamilast, tolafentrine, mesopram, drotaverine hydrochloride, lirimilast, roflumilast, cilomilast, oglemilast, apremilast, tetomilast, filaminast, (R)-(+)-4-[2-(3-Cyclopentyloxy-4-methoxyphenyl)-2-phenylethyl]pyridine (CDP-840), N-(3,5-Dichloro-4-pyridinyl)-2-[1-(4-fluorobenzyl)-5-hydroxy-1H-indol-3-yl]-2-oxoacetamide (GSK-842470), 9-(2-Fluorobenzyl)-N6-methyl-2-(trifluoromethyl)adenine (NCS-613), N-(3,5-Dichloro-4-pyridinyl)-8-methoxyquinoline-5-carboxamide (D-4418), 3-[3-(Cyclopentyloxy)-4-methoxybenzyl]-6-(ethylamino)-8-isopropyl-3H-purine hydrochloride (V-11294A), 6-[3-(N,N-Dimethylcarbamoyl)phenylsulfonyl]-4-(3-methoxyphenylamino)-8-methylquinoline-3-carboxamide hydrochloride (GSK-256066), 4-[6,7-Diethoxy-2,3-bis(hydroxymethyl)naphthalen-1-yl]-1-(2-methoxyethyl)pyridin-2(1H)-one (T-440), (−)-trans-2-[3′-[3-(N-Cyclopropylcarbamoyl)-4-oxo-1,4-dihydro-1,8-naphthyridin-1-yl]-3-fluorobiphenyl-4-yl]cyclopropanecarboxylic acid (MK-0873), CDC-801, UK-500001, BLX-914, 2-carbomethoxy-4-cyano-4-(3-cyclopropylmethoxy-4-difluororomethoxyphenyl)cyclohexan1-one, cis [4-cyano-4-(3-cyclopropylmethoxy-4-difluoromethoxyphenyl)cyclohexan-1-ol, GRC-4039, CDC-801, 5(S)-[3-(Cyclopentyloxy)-4-methoxyphenyl]-3(S)-(3-methylbenzyl)piperidin-2-one (IPL-455903), ONO-6126 (Eur Respir J 2003, 22(Suppl. 45): Abst 2557) and the salts claimed in the International Patent Applications Nos. WO03/097613, WO2004/058729, WO 2005/049581, WO 2005/123693 and WO 2005/123692.

Examples of suitable PI3Kδγ inhibitors that can be combined with the JAK inhibitors of the present invention are 2-Methyl-2-[4-[3-methyl-2-oxo-8-(3-quinolinyl)-2,3-dihydro-1H-imidazo[4,5-c]quinolin-1-yl]phenyl]propanenitrile (BEZ-235 from Novartis), CAL-101 (from Calistoga Pharmaceuticals) and N-Ethyl-N′-[3-(3,4,5-trimethoxyphenylamino)pyrido[2,3-b]pyrazin-6-yl]thiourea (AEZS-126 from Aeterna Zentaris).

The compounds of formula (I) and the combinations described herein may be used in the treatment of myeloproliferative disorders, leukemia, lymphoid malignancies and solid tumors; bone marrow and organ transplant rejection; immune-mediated diseases and inflammatory diseases, for example myeloproliferative disorders, leukemia, lymphoid malignancies and solid tumors; bone marrow and organ transplant rejection; and immune-mediated diseases; wherein the use of a JAK inhibitor is expected to have a beneficial effect, for example rheumatoid arthritis, multiple sclerosis, inflammatory bowel disease (such as ulcerative colitis or Crohn's disease), dry eye, uveitis, allergic conjunctivitis, allergic rhinitis, asthma, chronic obstructive pulmonary disease (COPD), atopic dermatitis and psoriasis. The compounds of formula (I) and the combinations described herein may also be used in the treatment of inflammatory diseases.

In one aspect, the compounds of formula (I) and the combinations described herein may be used in the treatment of myeloproliferative disorders, leukemia, lymphoid malignancies and solid tumors. In this aspect, the treatment is effected by inhibition of Janus Kinases in the subject. In another aspect, the compounds of formula (I) and the combinations described herein may be used in the treatment of bone marrow and organ transplant rejection; immune-mediated diseases and inflammatory diseases, for example from bone marrow and organ transplant rejection; and immune-mediated diseases, e.g. bone marrow and organ transplant rejection.

The treatment of these diseases and conditions is typically effected by inhibiting Janus Kinases (JAK) in the subject. The compounds of formula (I) and the combinations described herein may be used in the inhibition of Janus Kinases (JAK).

The active compounds in the combination may be administered together in the same pharmaceutical composition or in different compositions intended for separate, simultaneous, concomitant or sequential administration by the same or a different route.

It is contemplated that all active agents would be administered at the same time, or very close in time. Alternatively, one or two active agents could be taken in the morning and the other (s) later in the day. Or in another scenario, one or two active agents could be taken twice daily and the other (s) once daily, either at the same time as one of the twice-a-day dosing occurred, or separately. Preferably at least two, and more preferably all, of the active agents would be taken together at the same time. Preferably, at least two and more preferably all active agents would be administered as an admixture.

The invention is also directed to a combination product of the compounds described herein together with one or more other therapeutic agents for use in the treatment of a pathological condition or disease susceptible to amelioration by inhibition of Janus Kinases (JAK), in particular wherein the pathological condition or disease is selected from myeloproliferative disorders, leukemia, lymphoid malignancies and solid tumors; bone marrow and organ transplant rejection; immune-mediated diseases and inflammatory diseases, for example myeloproliferative disorders, leukemia, lymphoid malignancies and solid tumors; bone marrow and organ transplant rejection; and immune-mediated diseases. More particularly, the pathological condition or disease is selected from rheumatoid arthritis, multiple sclerosis, inflammatory bowel disease, dry eye, uveitis, allergic conjunctivitis, allergic rhinitis, asthma, chronic obstructive pulmonary disease (COPD), atopic dermatitis and psoriasis.

In one aspect, the combination product may be for the treatment of myeloproliferative disorders, leukemia, lymphoid malignancies and solid tumors. In this aspect, the treatment is effected by inhibition of Janus Kinases in the subject. In another aspect, the combination product may be used in the treatment of bone marrow and organ transplant rejection; immune-mediated diseases and inflammatory diseases, for example from bone marrow and organ transplant rejection; and immune-mediated diseases, e.g. bone marrow and organ transplant rejection.

The treatment of these diseases and conditions is typically effected by inhibiting Janus Kinases (JAK) in the subject. The combination product may be used in the inhibition of Janus Kinases (JAK).

The invention also encompasses the use of a combination of the compounds of the invention together with one or more other therapeutic agents for the manufacture of a formulation or medicament for treating these diseases.

The invention also provides a method of treatment of a pathological condition or disease susceptible to amelioration by inhibition of Janus Kinases (JAK), in particular wherein the pathological condition or disease is selected from myeloproliferative disorders, leukemia, lymphoid malignancies and solid tumors; bone marrow and organ transplant rejection; immune-mediated diseases and inflammatory diseases, for example myeloproliferative disorders, leukemia, lymphoid malignancies and solid tumors; bone marrow and organ transplant rejection; and immune-mediated diseases. More particularly, the pathological condition or disease is selected from rheumatoid arthritis, multiple sclerosis, inflammatory bowel disease, dry eye, uveitis, allergic conjunctivitis, allergic rhinitis, asthma, chronic obstructive pulmonary disease (COPD), atopic dermatitis and psoriasis; comprising administering a therapeutically effective amount of a combination of the compounds described herein together with one or more other therapeutic agents. In particular, the treatment is effected by inhibition of Janus Kinases in the subject.

The invention also provides a method of inhibiting Janus kinases in a subject in need thereof, which comprises administering to said subject a therapeutically effective amount of a combination of the compounds described herein together with one or more other therapeutic agents to a subject in need of such treatment.

The active compounds in the combinations of the invention may be administered by any suitable route, depending on the nature of the disorder to be treated, e.g. orally (as syrups, tablets, capsules, lozenges, controlled-release preparations, fast-dissolving preparations, etc); topically (as creams, ointments, lotions, nasal sprays or aerosols, etc); by injection (subcutaneous, intradermic, intramuscular, intravenous, etc.) or by inhalation (as a dry powder, a solution, a dispersion, etc).

The active compounds in the combination, i.e. the imidazopyridine derivatives of the invention, and the other optional active compounds may be administered together in the same pharmaceutical composition or in different compositions intended for separate, simultaneous, concomitant or sequential administration by the same or a different route.

One execution of the present invention consists of a kit of parts comprising an imidazopyridine derivative of the invention together with instructions for simultaneous, concurrent, separate or sequential use in combination with another active compound useful in the treatment of myeloproliferative disorders, leukemia, lymphoid malignancies and solid tumors; bone marrow and organ transplant rejection; immune-mediated diseases and inflammatory diseases, for example myeloproliferative disorders, leukemia, lymphoid malignancies and solid tumors; bone marrow and organ transplant rejection; and immune-mediated diseases, and more particularly, useful in the treatment of rheumatoid arthritis, multiple sclerosis, inflammatory bowel disease, dry eye, uveitis, allergic conjunctivitis, allergic rhinitis, asthma, chronic obstructive pulmonary disease (COPD), atopic dermatitis and psoriasis.

Another execution of the present invention consists of a package comprising an imidazopyridine derivative of the invention and another active compound useful in the treatment of myeloproliferative disorders, leukemia, lymphoid malignancies and solid tumors; bone marrow and organ transplant rejection; immune-mediated diseases and inflammatory diseases, for example myeloproliferative disorders, leukemia, lymphoid malignancies and solid tumors; bone marrow and organ transplant rejection; and immune-mediated diseases, and more particularly, useful in the treatment of rheumatoid arthritis, multiple sclerosis, inflammatory bowel disease, dry eye, uveitis, allergic conjunctivitis, allergic rhinitis, asthma, chronic obstructive pulmonary disease (COPD), atopic dermatitis and psoriasis.

### Pharmaceutical Compositions

Pharmaceutical compositions according to the present invention comprise the compounds of the invention in association with a pharmaceutically acceptable diluent or carrier.

The invention further provides pharmaceutical compositions comprising the compounds of the invention in association with a pharmaceutically acceptable diluent or carrier together with one or more other therapeutic agents for use in the treatment of a pathological condition or disease susceptible to amelioration by inhibition of Janus Kinases (JAK), such as the ones previously described.

The invention is also directed to pharmaceutical compositions for use in the treatment of a pathological condition or disease susceptible to amelioration by inhibition of Janus Kinases (JAK), in particular wherein the pathological condition or disease is selected from myeloproliferative disorders, leukemia, lymphoid malignancies and solid tumors; bone marrow and organ transplant rejection; immune-mediated diseases and inflammatory diseases, for example myeloproliferative disorders, leukemia, lymphoid malignancies and solid tumors; bone marrow and organ transplant rejection; and immune-mediated diseases, and more particularly, wherein the pathological condition or disease is selected from rheumatoid arthritis, multiple sclerosis, inflammatory bowel disease, dry eye, uveitis, allergic conjunctivitis, allergic rhinitis, asthma, chronic obstructive pulmonary disease (COPD), atopic dermatitis and psoriasis. The invention also encompasses the use of a pharmaceutical composition of the invention for the manufacture of a medicament for treating these diseases.

In one aspect, the pharmaceutical composition may be for the treatment of myeloproliferative disorders, leukemia, lymphoid malignancies and solid tumors. In this aspect, the treatment is effected by inhibition of Janus Kinases in the subject. In another aspect, the pharmaceutical composition may be used in the treatment of bone marrow and organ transplant rejection; immune-mediated diseases and inflammatory diseases, for example from bone marrow and organ transplant rejection; and immune-mediated diseases, e.g. bone marrow and organ transplant rejection.

The treatment of these diseases and conditions is typically effected by inhibiting Janus Kinases (JAK) in the subject. The pharmaceutical composition may be used in the inhibition of Janus Kinases (JAK).

The invention also provides a method of treatment of a pathological condition or disease susceptible to amelioration by inhibition of Janus Kinases (JAK), in particular wherein the pathological condition or disease is selected from myeloproliferative disorders, leukemia, lymphoid malignancies and solid tumors; bone marrow and organ transplant rejection; immune-mediated diseases and inflammatory diseases, for example myeloproliferative disorders, leukemia, lymphoid malignancies and solid tumors; bone marrow and organ transplant rejection; and immune-mediated diseases, and more particularly, wherein the pathological condition or disease is selected from rheumatoid arthritis, multiple sclerosis, inflammatory bowel disease, dry eye, uveitis, allergic conjunctivitis, allergic rhinitis, asthma, chronic obstructive pulmonary disease (COPD), atopic dermatitis and psoriasis, comprising administering a therapeutically effective amount of a pharmaceutical composition as defined herein. In particular, the treatment is effected by inhibition of Janus Kinases in the subject.

The invention also provides a method of inhibiting Janus kinases in a subject in need thereof, which comprises administering to said subject a therapeutically effective amount of a pharmaceutical composition as defined herein to a subject in need of such treatment.

The present invention also provides pharmaceutical compositions which comprise, as an active ingredient, at least an imidazopyridine of formula (I) or a pharmaceutically acceptable salt thereof in association with a pharmaceutically acceptable excipient such as a carrier or diluent. The active ingredient may comprise 0.001% to 99% by weight, preferably 0.01% to 90% by weight, of the composition depending upon the nature of the formulation and whether further dilution is to be made prior to application.

Preferably the compositions are made up in a form suitable for oral, inhalation, topical, nasal, rectal, percutaneous or injectable administration.

The pharmaceutically acceptable excipients which are admixed with the active compound or salts of such compound, to form the compositions of this invention are well-known per se and the actual excipients used depend inter alia on the intended method of administering the compositions.

Compositions for oral administration may take the form of tablets, retard tablets, sublingual tablets, capsules, inhalation aerosols, inhalation solutions, dry powder inhalation, or liquid preparations, such as mixtures, elixirs, syrups or suspensions, all containing the compound of the invention; such preparations may be made by methods well-known in the art.

The diluents which may be used in the preparation of the compositions include those liquid and solid diluents which are compatible with the active ingredient, together with colouring or flavouring agents, if desired. Tablets or capsules may conveniently contain between 0.01-3000 mg, more preferably 0.5-1000 mg of active ingredient or the equivalent amount of a pharmaceutically acceptable salt thereof.

The liquid composition adapted for oral use may be in the form of solutions or suspensions. The solutions may be aqueous solutions of a soluble salt or other derivative of the active compound in association with, for example, sucrose to form a syrup. The suspensions may comprise an insoluble active compound of the invention or a pharmaceutically acceptable salt thereof in association with water, together with a suspending agent or flavouring agent.

Compositions for parenteral injection may be prepared from soluble salts, which may or may not be freeze-dried and which may be dissolved in pyrogen free aqueous media or other appropriate parenteral injection fluid.

Compositions for topical administration may take the form of ointments, creams or lotions, all containing the compound of the invention; such preparations may be made by methods well-known in the art.

Effective doses are normally in the range of 0.01-3000 mg, more preferably 0.5-1000 mg of active ingredient or the equivalent amount of a pharmaceutically acceptable salt thereof per day. Daily dosage may be administered in one or more treatments, preferably from 1 to 4 treatments, per day.

The pharmaceutical compositions may conveniently be presented in unit dosage form and may be prepared by any of the methods well known in the art of pharmacy.

The pharmaceutical compositions of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A syrup formulation will generally consist of a suspension or solution of the compound or salt in a liquid carrier for example, ethanol, peanut oil, olive oil, glycerine or water with flavouring or colouring agent.

Where the composition is in the form of a tablet, any pharmaceutical carrier routinely used for preparing solid formulations may be used. Examples of such carriers include magnesium stearate, talc, gelatine, acacia, stearic acid, starch, lactose and sucrose.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent.

Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Where the composition is in the form of a capsule, any routine encapsulation is suitable, for example using the aforementioned carriers in a hard gelatine capsule. Where the composition is in the form of a soft gelatine capsule any pharmaceutical carrier routinely used for preparing dispersions or suspensions may be considered, for example aqueous gums, celluloses, silicates or oils, and are incorporated in a soft gelatine capsule.

Dry powder compositions for topical delivery to the lung by inhalation may, for example, be presented in capsules and cartridges of for example gelatine or blisters of for example laminated aluminium foil, for use in an inhaler or insufflator. Formulations generally contain a powder mix for inhalation of the compound of the invention and a suitable powder base (carrier substance) such as lactose or starch. Use of lactose is preferred. Each capsule or cartridge may generally contain between 2 μg and 150 μg of each therapeutically active ingredient. Alternatively, the active ingredient (s) may be presented without excipients.

Packaging of the formulation for inhalation may be carried out by using suitable inhaler devices such as Genuair® (formerly Novolizer® SD2FL) which is described in the following patent applications: WO 97/000703, WO 03/000325 and WO 2006/008207.

Typical compositions for nasal delivery include those mentioned above for inhalation and further include non-pressurized compositions in the form of a solution or suspension in an inert vehicle such as water optionally in combination with conventional excipients such as buffers, anti-microbials, tonicity modifying agents and viscosity modifying agents which may be administered by nasal pump.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

The amount of each active which is required to achieve a therapeutic effect will, of course, vary with the particular active, the route of administration, the subject under treatment, and the particular disorder or disease being treated.

The following preparations forms are cited as formulation examples:

### COMPOSITION EXAMPLE 1

50,000 capsules, each containing 100 mg of 3-((3R)-3-{[2-(6-fluoroimidazo[1,2-a]pyridin-3-yl)pyrimidin-4-yl]amino}piperidin-1-yl)-3-oxopropanenitrile (active ingredient), were prepared according to the following formulation:

| | |
|---|---|
| Active ingredient | 5 Kg |
| Lactose monohydrate | 10 Kg |
| Colloidal silicon dioxide | 0.1 Kg |
| Corn starch | 1 Kg |
| Magnesium stearate | 0.2 Kg |

### Procedure

The above ingredients were sieved through a 60 mesh sieve, and were loaded into a suitable mixer and filled into 50,000 gelatine capsules.

### COMPOSITION EXAMPLE 2

50,000 tablets, each containing 50 mg of 3-((3R)-3-{[2-(6-fluoroimidazo[1,2-a]pyridin-3-yl)pyrimidin-4-yl]amino}piperidin-1-yl)-3-oxopropanenitrile (active ingredient), were prepared from the following formulation:

| | |
|---|---|
| Active ingredient | 2.5 Kg |
| Microcrystalline cellulose | 1.95 Kg |
| Spray dried lactose | 9.95 Kg |
| Carboxymethyl starch | 0.4 Kg |
| Sodium stearyl fumarate | 0.1 Kg |
| Colloidal silicon dioxide | 0.1 Kg |

### Procedure

All the powders were passed through a screen with an aperture of 0.6 mm, then mixed in a suitable mixer for 20 minutes and compressed into 300 mg tablets using 9 mm disc and flat bevelled punches. The disintegration time of the tablets was about 3 minutes.

Modifications, which do not affect, alter, change or modify the essential aspects of the compounds, combinations or pharmaceutical compositions described, are included within the scope of the present invention.

1. A method of treating a pathological condition or disease susceptible to amelioration by inhibition of Janus Kinases (JAK) comprising administering to a subject in need thereof an effective amount of a compound of formula (I): 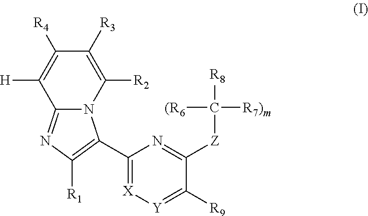
wherein,
m is 0 or an integer from 1 to 3;
Z is an oxygen atom or NR₅;
X and Y are independently chosen from a nitrogen atom and —CR₉, wherein at least one of X and Y is a nitrogen atom;
R₁, R₂, R₃, R₄ and R₉ are each independently chosen from a hydrogen atom, halogen atoms, cyano, linear or branched C₁-C₆ alkyl, C₂-C₄ alkenyl, C₂-C₄ alkynyl, C₁-C₄ haloalkyl, C₁-C₄ hydroxyalkyl, C₃-C₁₀ cycloalkyl, C₃-C₁₀ cycloalkenyl, (C₁-C₄ alkyl)(C₃-C₇) cycloalkyl, monocyclic or polycyclic C₅-C₁₄ aryl, 5- to 14-membered heteroaryl containing at least one heteroatom selected from O, S and N, 5- to 14-membered heterocyclyl containing at least one heteroatom selected from O, S and N, bicyclyl containing a monocyclic C₅-C₉ aryl or heteroaryl group bonded directly to a 5- to 9-membered cycloalkyl or heterocyclyl, said heteroaryl or heterocyclyl containing at least one heteroatom selected from O, S and N, aza-bicycloalkyl having up to 12 carbon atoms and aza-bicycloalkenyl having up to 12 carbon atoms,
wherein the alkenyl, alkynyl, haloalkyl, hydroxyalkyl, cycloalkyl, cycloalkenyl, aryl, heteroaryl, heterocyclyl, bicyclyl, aza-bicycloalkyl and aza-bicycloalkenyl are independently unsubstituted or substituted by one or more Ra, and the alkyl groups are independently unsubstituted or substituted with one or more Rb;
or R₁, R₂, R₃, R₄ and R₉ are each independently chosen from —SR₁₃, —SOR₁₃, —S(O)₂R₁₃, —S(O)₂NR₁₃R₁₄, —NR₁₃S(O)₂R₁₄, —NR₁₃S(O)₂NR₁₄, —OR₁₃, —C(O)OR₁₃, —O—C(O)R₁₃, —C(O)—(CH₂)ₙ—R₁₃, —NR₁₃R₁₄, —C(O)—(CH₂)ₙ—NR₁₃R₁₄, —NR₁₃C(O)—(CH₂)ₙ—R₁₄ and —NR₁₃C(O)—(CH₂)ₙ—NR₁₄R₁₅, wherein each n is independently 0, 1 or 2;
or when two adjacent —CR₉ groups are present, both —CR₉ and the carbon atoms to which they are bonded optionally form a C₅-C₁₂ aryl group or a 4- to 12-membered heteroaryl, cycloalkyl or heterocyclyl group, said heteroaryl and heterocyclyl containing at least one heteroatom selected from O, S and N, the aryl, heteroaryl, cycloalkyl and heterocyclyl being independently unsubstituted or substituted by one or more substituents selected from halogen atoms, linear or branched C₁-C₆ alkyl, monocyclic or polycyclic C₅-C₁₄ aryl, 5- to 14-membered heteroaryl containing at least one heteroatom selected from O, S and N, and 5- to 14-membered heterocyclyl containing at least one heteroatom selected from O, S and N, wherein the alkyl, the aryl, the heteroaryl and the heterocyclyl substituents are independently unsubstituted or substituted by one or more substituents selected from halogen atoms, hydroxyl, cyano, linear or branched C₁-C₆ alkyl and C₁-C₄ haloalkyl;
R₅ is chosen from a hydrogen atom, linear or branched C₁-C₆ alkyl optionally substituted by one or more substituents selected from hydroxyl, cyano, C₁-C₄ haloalkyl, C₁-C₄ hydroxyalkyl, C₃-C₁₀ cycloalkyl, phenyl and 6 membered, saturated N-containing heterocyclyl ring, or R₅ is chosen from —S(O)₂R₁₀, —S(O)₂NR₁₀R₁₁, —C(O)OR₁₀, —C(O)—(CH₂)ₙ—R₁₀, and —C(O)—(CH₂)ₙ—NR₁₀R₁₁;
R₆ and R₇ are each independently chosen from a hydrogen atom or linear or branched C₁-C₆ alkyl independently optionally substituted by one or more substituents selected from hydroxyl, cyano, C₁-C₄ haloalkyl, C₁-C₄ hydroxyalkyl, C₁-C₄ alkoxy, C₁-C₄ alkoxycarbonyl, C₃-C₇ cycloalkyl, phenyl and 6 membered, saturated N-containing heterocyclyl ring;
R₈ is chosen from a hydrogen atom, halogen atoms, cyano, linear or branched C₁-C₆ alkyl, C₂-C₄ alkenyl, C₂-C₄ alkynyl, C₁-C₄ haloalkyl, C₁-C₄ hydroxyalkyl, —(C₁-C₆ alkyl)-(C₁-C₄ alkoxy), C₃-C₁₀ cycloalkyl, C₃-C₁₀ cycloalkenyl, monocyclic or polycyclic C₅-C₁₄ aryl, 5- to 14-membered heteroaryl containing at least one heteroatom selected from O, S and N, 5- to 14-membered heterocyclyl containing at least one heteroatom selected from O, S and N, C₃-C₇ heterocycloalkyl ketone containing 1, 2 or 3 nitrogen atoms, bicyclyl containing a monocyclic C₅-C₉ aryl or heteroaryl bonded directly to a 5- to 9-membered cycloalkyl or heterocyclyl, said heteroaryl or heterocyclyl containing at least one heteroatom selected from O, S and N, aza-bicycloalkyl having up to 12 carbon atoms and aza-bicycloalkenyl having up to 12 carbon atoms,
wherein the alkenyl, alkynyl, haloalkyl, hydroxyalkyl, cycloalkyl, cycloalkenyl, aryl, heteroaryl, heterocyclyl, heterocycloalkyl ketone, bicyclyl, aza-bicycloalkyl and aza-bicycloalkenyl are independently unsubstituted or substituted by one or more substituents selected from Ra, —(C₁-C₄ alkyl)-CN, and —(C₁-C₄ alkyl)-C(O)NR′R″ wherein R′ and R″ are the same or different and are independently selected from hydrogen atoms and linear or branched C₁-C₄ alkyl; and the alkyl groups are independently unsubstituted or substituted by one or more Rb; or R₈ is chosen from —SR₁₃, —SOR₁₃, —S(O)₂R₁₃, —S(O)₂NR₁₃R₁₄, —NR₁₃S(O)₂R₁₄, —NR₁₃S(O)₂NR₁₄, —OR₁₃, —C(O)OR₁₃, —O—C(O)R₁₃, —C(O)—(CH₂)ₙ—R₁₃, —NR₁₃R₁₄, —C(O)—(CH₂)ₙ—NR₁₃R₁₄, —NR₁₃C(O)—(CH₂)ₙ—R₁₄, and —NR₁₃C(O)—(CH₂)ₙ—NR₁₄R₁₅, wherein n is independently 0, 1 or 2,
or R₈ together with R₅ and the nitrogen atom to which R₅ is bonded form a 4- to 10-membered, saturated heterocyclyl group, which contains, as heteroatoms, one or two nitrogen atoms and which is substituted by linear or branched C₁-C₆ alkyl, monocyclic or polycyclic C₅-C₁₄ aryl, 5- to 14-membered heteroaryl containing at least one heteroatom selected from O, S and N, 5- to 14-membered heterocyclyl containing at least one heteroatom selected from O, S and N, —SOR₁₀, —C(O)—(CH₂)ₙ—R₁₀, or —C(O)—(CH₂)ₙ—NR₁₀R₁₁, wherein each n is independently 0, 1 or 2,
wherein the alkyl, aryl, heteroaryl and heterocyclyl are independently unsubstituted or substituted by one or more substituents selected from halogen atoms, hydroxyl, cyano, linear or branched C₁-C₆ alkyl and C₁-C₄ haloalkyl, and wherein the alkyl groups are independently unsubstituted or substituted by one or more substituents selected from halogen atoms, hydroxyl, cyano and C₁-C₄ haloalkyl;
provided that when m is zero, R₈ is other than —SR₁₃, —SOR₁₀, —S(O)₂R₁₃, —S(O)₂N₁₃R₁₄, —NR₁₃S(O)₂R₁₄, —NR₁₃S(O)₂NR₁₄, —OR₁₃, —O—C(O)R₁₃, —NR₁₃R₁₄, —NR₁₃C(O)—(CH₂)ₙ—R₁₄, or —NR₁₃C(O)—(CH₂)ₙ—NR₁₄R₁₅,
wherein each Ra is independently chosen from halogen atoms, cyano, hydroxyl, linear or branched C₁-C₆ alkyl, which alkyl is independently unsubstituted or substituted by one or more cyano, C₁-C₄ haloalkyl, C₁-C₄ hydroxyalkyl, C₃-C₇ cycloalkyl or C₃-C₇ cycloalkenyl, monocyclic or polycyclic C₅-C₁₄ aryl, which aryl is independently unsubstituted or substituted by one or more substituents selected from halogen atoms or cyano, 5- to 14-membered heteroaryl containing at least one heteroatom selected from O, S and N, which heteroaryl is independently unsubstituted or substituted by one or more substituents selected from halogen atoms or cyano, 5- to 14-membered heterocyclyl containing at least one heteroatom selected from O, S and N, —SR₁₀, —SOR₁₀, —S(O)₂R₁₀, —S(O)₂NR₁₀R₁₁, —NR₁₀S(O)₂R₁₁, —NR₁₀S(O)₂NR₁₁, —OR₁₀, —C(O)OR₁₀, —C(O)—CH₂—OR₁₀, —C(O)—(CH₂)ₙ—O—(CH₂)ₚR₁₀, —C(O)—(CH₂)ₙ—O—(CH₂)ₚO—R₁₀, —O—C(O)R₁₀, —C(O)—(CH₂)—R₁₀, —NR₁₀R₁₁, —C(O)—(CH₂)ₙ—NR₁₀R₁₁, —R₁₀C(O)—(CH₂)ₙ—NR₁₁R₁₂, —C(C₁-C₄ alkyl)₂-C(O)—(CH₂)ₙ—NR₁₀R₁₁, —NR₁₀C(O)—(CH₂)ₙ—R₁₁ and —NR₁₀C(O)—(CH₂)ₙ—NR₁₁R₁₂, wherein each n is independently 0 or an integer from 1 to 5, and wherein p is an integer from 1 to 3;
each Rb is independently chosen from cyano, hydroxyl, linear or branched C₁-C₆ alkyl, C₁-C₄ haloalkyl, C₁-C₄ hydroxyalkyl, C₃-C₇ cycloalkyl and C₃-C₇ cycloalkenyl, monocyclic or polycyclic C₅-C₁₄ aryl, which aryl is independently unsubstituted or substituted by one or more substituents selected from halogen atoms or cyano, 5- to 14-membered heteroaryl containing at least one heteroatom selected from O, S and N, which heteroaryl is independently unsubstituted or substituted by one or more substituents selected from halogen atoms or cyano, 5- to 14-membered heterocyclyl containing at least one heteroatom selected from O, S and N, —SR₁₀, —SOR₁₀, —S(O)₂R₁₀, —S(O)₂NR₁₀R₁₁, —NR₁₀S(O)₂R₁₁, —NR₁₀S(O)₂NR₁₁, —OR₁₀, —C(O)OR₁₀, —C(O)—CH₂—OR₁₀, —C(O)—(CH₂)ₙ—O—(CH₂)ₚR₁₀, —C(O)—(CH₂)ₙ—O—(CH₂)ₚO—R₁₀, —O—C(O)R₁₀, —C(O)—(CH₂)ₙ—R₁₀, —NR₁₀R₁₁, —C(O)—(CH₂)ₙ—NR₁₀R₁₁, —R₁₀C(O)—(CH₂)ₙ—R₁₁R₁₂, —C(C₁-C₄ alkyl)₂-C(O)—(CH₂)ₙ—NR₁₀R₁₁, —NR₁₀C(O)—(CH₂)ₙ—R₁₁ and —NR₁₀C(O)—(CH₂)ₙ—NR₁₁R₁₂, wherein each n is independently 0 or an integer from 1 to 5, and wherein p is independently an integer from 1 to 3;
R₁₀, R₁₁ and R₁₂ are each independently chosen from a hydrogen atom, cyano, linear or branched C₁-C₆ alkyl, C₁-C₄ haloalkyl, C₁-C₆ hydroxyalkyl, C₁-C₄ alkoxycarbonyl, C₃-C₇ cycloalkyl, phenyl, —(C₁-C₄ hydroxyalkyl)-(phenyl), —C(O)-(phenyl), 5- to 6-membered monocyclic heteroaryl containing 1, 2, 3 or 4 heteroatoms selected from N, O and S, 5- to 6-membered, heterocyclyl containing 1, 2 or 3 heteroatoms selected from oxygen and nitrogen atoms, C₃-C₇ heterocycloalkyl ketone containing 1, 2 or 3 nitrogen atoms, bicyclyl containing monocyclic C₅-C₆ aryl or heteroaryl bonded directly to 5- to 6-membered cycloalkyl or heterocyclyl which heteroaryl or heterocyclyl contains 1, 2 or 3 heteroatoms selected from oxygen and nitrogen atoms, the haloalkyl, hydroxyalkyl, alkoxycarbonyl, cycloalkyl, phenyl, hydroxyalkyl-phenyl, heteroaryl, heterocyclyl, heterocycloalkyl ketone and bicyclyl being independently unsubstituted or substituted by one or more Rc, and the alkyl groups are independently unsubstituted or substituted by one or more Rd;
each Rc is independently chosen from halogen atoms, hydroxyl, cyano, linear or branched C₁-C₆ alkyl, C₁-C₄ haloalkyl, C₁-C₄ alkoxy, C₁-C₄ hydroxyalkyl, C₃-C₇ cycloalkyl, phenyl, 5- to 6-membered monocyclic heteroaryl containing 1, 2 or 3 nitrogen atoms, 5- to 6-membered heterocyclyl containing 1, 2 or 3 nitrogen atoms, and C₃-C₇ heterocycloalkyl ketone containing 1, 2 or 3 nitrogen atoms, said phenyl being independently unsubstituted or substituted with one or more halogen atoms, and said heteroaryl, heterocyclyl and heterocycloalkyl ketone being independently unsubstituted or substituted by one or more linear or branched C₁-C₃ alkyl;
each Rd is independently chosen from cyano, C₁-C₄ haloalkyl, C₁-C₄ alkoxy, C₁-C₄ hydroxyalkyl, C₃-C₇ cycloalkyl, phenyl, 5- to 6-membered monocyclic heteroaryl containing 1, 2 or 3 nitrogen atoms, 5- to 6-membered heterocyclyl containing 1, 2 or 3 nitrogen atoms, and C₃-C₇ heterocycloalkyl ketone containing 1, 2 or 3 nitrogen atoms, said phenyl being independently unsubstituted or substituted with one or more halogen atoms, and said heteroaryl, heterocyclyl and heterocycloalkyl ketone being independently unsubstituted or substituted by one or more linear or branched C₁-C₃ alkyl;
R₁₃, R₁₄, and R₁₅ are each independently chosen from a hydrogen atom, cyano, linear or branched C₁-C₆ alkyl, C₁-C₄ haloalkyl, C₁-C₄ hydroxyalkyl, —(C₁-C₆ alkyl)-(C₁-C₄ alkoxy), —(C₁-C₆ alkyl)-(5- to 7-membered heterocyclyl containing at least one heteroatom selected from O, S and N), C₁-C₄ alkoxycarbonyl, C₃-C₇ cycloalkyl, monocyclic or polycyclic C₅-C₁₄ aryl, 5- to 14-membered heteroaryl containing at least one heteroatom selected from O, S and N, and 5- to 14-membered heterocyclyl containing at least one heteroatom selected from O, S and N, wherein the haloalkyl, hydroxyalkyl, alkoxycarbonyl, cycloalkyl, aryl, heteroaryl and heterocyclyl are independently unsubstituted or substituted by one or more Ra, and the alkyl groups are independently optionally substituted by one or more Rb.

2. The method according to claim 1, wherein
R₁, R₂, R₃, R₄ and R₉ are each independently chosen from a hydrogen atom, halogen atoms, cyano, linear or branched C₁-C₆ alkyl, C₂-C₄ alkenyl, C₂-C₄ alkynyl, C₁-C₄ haloalkyl, C₁-C₄ hydroxyalkyl, C₃-C₁₀ cycloalkyl, C₃-C₁₀ cycloalkenyl, monocyclic or polycyclic C₅-C₁₄ aryl, 5- to 14-membered heteroaryl containing at least one heteroatom selected from O, S and N, 5- to 14-membered heterocyclyl containing at least one heteroatom selected from O, S and N, bicyclyl containing monocyclic C₅-C₉ aryl or heteroaryl bonded directly to a 5- to 9-membered cycloalkyl or heterocyclyl, said heteroaryl or heterocyclyl containing at least one heteroatom selected from O, S and N, an aza-bicycloalkyl having up to 12 carbon atoms and an aza-bicycloalkenyl having up to 12 carbon atoms,
wherein the alkenyl, alkynyl, haloalkyl, hydroxyalkyl, cycloalkyl, cycloalkenyl, aryl, heteroaryl, heterocyclyl, bicyclyl, aza-bicycloalkyl and aza-bicycloalkenyl are independently unsubstituted or substituted by one or more Ra, and the alkyl groups are independently unsubstituted or substituted with one or more Rb;
or R₁, R₂, R₃, R₄ and R₉ are each independently chosen from —SR₁₃, —SOR₁₃, —S(O)₂R₁₃—S(O)₂NR₁₃R₁₄, —NR₁₃S(O)₂R₁₄, —NR₁₃S(O)₂NR₁₄, —OR₁₃, —C(O)OR₁₃, —O—C(O)R₁₃, —C(O)—(CH₂)ₙ—R₁₃, —NR₁₃R₁₄, —C(O)—(CH₂)ₙ—NR₁₃R₁₄, —NR₁₃C(O)—(CH₂)ₙ—R₁₄ and —NR₁₃C(O)—(CH₂)ₙ—NR₁₄R₁₅, wherein each n is independently 0, 1 or 2;
or when two adjacent —CR₉ groups are present, both —CR₉ and the carbon atoms to which they are bonded optionally form a C₅-C₁₂ aryl group or a 4- to 12-membered heteroaryl, cycloalkyl or heterocyclyl, said heteroaryl and heterocyclyl containing at least one heteroatom selected from O, S and N, the aryl, heteroaryl, cycloalkyl and heterocyclyl being independently unsubstituted or substituted by one or more substituents selected from halogen atoms, linear or branched C₁-C₆ alkyl, monocyclic or polycyclic C₅-C₁₄ aryl, 5- to 14-membered heteroaryl containing at least one heteroatom selected from O, S and N, and 5- to 14-membered heterocyclyl containing at least one heteroatom selected from O, S and N, wherein the alkyl, the aryl, the heteroaryl and the heterocyclyl substituents are independently unsubstituted or substituted by one or more substituents selected from halogen atoms, hydroxyl, cyano, linear or branched C₁-C₆ alkyl and C₁-C₄ haloalkyl;
R₅ is chosen from a hydrogen atom, linear or branched C₁-C₆ alkyl optionally substituted by one or more substituents selected from hydroxyl, cyano, C₁-C₄ haloalkyl, C₁-C₄ hydroxyalkyl, C₃-C₁₀ cycloalkyl, phenyl and 6 membered, saturated N-containing heterocyclyl ring, or R₅ is chosen from —S(O)₂R₁₀, —S(O)₂NR₁₀R₁₁, —C(O)OR₁₀, —C(O)—(CH₂)ₙ—R₁₀, and —C(O)—(CH₂)ₙ—NR₁₀R₁₁;
R₆ and R₇ are each independently chosen from a hydrogen atom or linear or branched C₁-C₆ alkyl optionally substituted by one or more substituents selected from hydroxyl, cyano, C₁-C₄ haloalkyl, C₁-C₄ hydroxyalkyl, C₁-C₄ alkoxycarbonyl, C₃-C₇ cycloalkyl, phenyl and 6 membered, saturated N-containing heterocyclyl ring;
R₈ is chosen from a hydrogen atom, halogen atoms, cyano, linear or branched C₁-C₆ alkyl, C₂-C₄ alkenyl, C₂-C₄ alkynyl, C₁-C₄ haloalkyl, C₁-C₄ hydroxyalkyl, C₃-C₁₀ cycloalkyl, C₃-C₁₀ cycloalkenyl, monocyclic or polycyclic C₆-C₁₄ aryl, 5- to 14-membered heteroaryl containing at least one heteroatom selected from O, S and N, 5- to 14-membered heterocyclyl containing at least one heteroatom selected from O, S and N, bicyclyl containing a monocyclic C₅-C₉ aryl or heteroaryl bonded directly to a 5- to 9-membered cycloalkyl or heterocyclyl, said heteroaryl or heterocyclyl containing at least one heteroatom selected from O, S and N, an aza-bicycloalkyl having up to 12 carbon atoms and aza-bicycloalkenyl having up to 12 carbon atoms,
wherein the alkenyl, alkynyl, haloalkyl, hydroxyalkyl, cycloalkyl, cycloalkenyl, aryl, heteroaryl, heterocyclyl, bicyclyl, aza-bicycloalkyl and aza-bicycloalkenyl are independently unsubstituted or substituted by one or more substituents selected from Ra, —(C₁-C₄ alkyl)-CN, and —(C₁-C₄ alkyl)-C(O)NR′R″ wherein R′ and R″ are the same or different and are independently selected from hydrogen atoms and linear or branched C₁-C₄ alkyl; and the alkyl groups are independently unsubstituted or substituted by one or more Rb;
or R₈ is chosen from —SR₁₃, —SOR₁₃, —S(O)₂R₁₃, —S(O)₂NR₁₃R₁₄, —NR₁₃S(O)₂R₁₄, —NR₁₃S(O)₂NR₁₄, —OR₁₃, —C(O)OR₁₃, —O—C(O)R₁₃, —C(O)—(CH₂)ₙ—R₁₃, —NR₁₃R₁₄, —C(O)—(CH₂)ₙ—NR₁₃R₁₄, —NR₁₃C(O)—(CH₂)ₙ—R₁₄, and —NR₁₃C(O)—(CH₂)ₙ—NR₁₄R₁₅, wherein n is independently 0, 1 or 2,
or R₈ together with R₅ and the nitrogen atom to which R₅ is bonded form a 4- to 10-membered, saturated heterocyclyl group, which contains, as heteroatoms, one or two nitrogen atoms and which is substituted by linear or branched C₁-C₆ alkyl, monocyclic or polycyclic C₅-C₁₄ aryl, 5- to 14-membered heteroaryl containing at least one heteroatom selected from O, S and N, 5- to 14-membered heterocyclyl containing at least one heteroatom selected from O, S and N, —SOR₁₀, —C(O)—(CH₂)ₙ—R₁₀, and —C(O)—(CH₂)ₙ—NR₁₀R₁₁, wherein each n is independently 0, 1 or 2,
wherein the alkyl, aryl, heteroaryl and heterocyclyl are independently unsubstituted or substituted by one or more substituents selected from halogen atoms, hydroxyl, cyano, linear or branched C₁-C₆ alkyl and C₁-C₄ haloalkyl, and wherein the alkyl groups are independently unsubstituted or substituted by one or more substituents selected from halogen atoms, hydroxyl, cyano and C₁-C₄ haloalkyl;
provided that when m is zero, R₈ is other than —SR₁₃, —SOR₁₃, —S(O)₂R₁₃, —S(O)₂NR₁₃R₁₄, —NR₁₃S(O)₂R₁₄, —NR₁₃S(O)₂NR₁₄, —OR₁₃, —O—C(O)R₁₃, —NR₁₃R₁₄, —NR₁₃C(O)—(CH₂)ₙ—R₁₄, and —NR₁₃C(O)—(CH₂)ₙ—NR₁₄R₁₅,
wherein each Ra is independently chosen from halogen atoms, cyano, hydroxyl, linear or branched C₁-C₆ alkyl, C₁-C₄ haloalkyl, C₁-C₄ hydroxyalkyl, C₃-C₇ cycloalkyl or C₃-C₇ cycloalkenyl, monocyclic or polycyclic C₅-C₁₄ aryl, which aryl is independently unsubstituted or substituted by one or more halogen atoms, 5- to 14-membered heteroaryl containing at least one heteroatom selected from O, S and N, 5- to 14-membered heterocyclyl containing at least one heteroatom selected from O, S and N, —SR₁₀, —SOR₁₀, —S(O)₂R₁₀, —S(O)₂NR₁₀R₁₁, —NR₁₀S(O)₂R₁₁, —NR₁₀S(O)₂NR₁₁, —OR₁₀, —C(O)OR₁₀, —O—C(O)R₁₀, —C(O)—(CH₂)ₙ—R₁₀, —NR₁₀R₁₁, —C(O)—(CH₂)ₙ—NR₁₀R₁₁, —NR₁₀C(O)—(CH₂)ₙ—R₁₁ and —NR₁₀C(O)—(CH₂)ₙ—NR₁₁R₁₂, wherein each n is independently 0, 1 or 2;
each Rb is independently chosen from cyano, C₁-C₄ haloalkyl, C₁-C₄ hydroxyalkyl, C₃-C₇ cycloalkyl or C₃-C₇ cycloalkenyl, monocyclic or polycyclic C₅-C₁₄ aryl, which aryl is independently unsubstituted or substituted by one or more halogen atoms, 5- to 14-membered heteroaryl containing at least one heteroatom selected from O, S and N, 5- to 14-membered heterocyclyl containing at least one heteroatom selected from O, S and N, —SR₁₀, —SOR₁₀, —S(O)₂R₁₀, —S(O)₂NR₁₀R₁₁, —NR₁₀S(O)₂R₁₁, —NR₁₀S(O)₂NR₁₁, —OR₁₀, —C(O)OR₁₀, —O—C(O)R₁₀, —C(O)—(CH₂)ₙ—R₁₀, —NR₁₀R₁₁, —C(O)—(CH₂)ₙ—NR₁₀R₁₁, —NR₁₀C(O)—(CH₂)ₙ—R₁₁ and —NR₁₀C(O)—(CH₂)ₙ—NR₁₁R₁₂, wherein n is independently 0, 1 or 2;
R₁₀, R₁₁ and R₁₂ are each independently chosen from a hydrogen atom, cyano, linear or branched C₁-C₆ alkyl, C₁-C₄ haloalkyl, C₁-C₄ hydroxyalkyl, C₁-C₄ alkoxycarbonyl, C₃-C₇ cycloalkyl, phenyl, 5- to 6-membered monocyclic heteroaryl containing 1, 2 or 3 heteroatoms selected from N, O and S, 5- to 6-membered, heterocyclyl containing 1, 2 or 3 nitrogen atoms, bicyclyl containing a monocyclic C₅-C₆ aryl or heteroaryl bonded directly to a 5- to 6-membered cycloalkyl or heterocyclyl which heteroaryl or heterocyclyl contains 1, 2 or 3 nitrogen atoms, the haloalkyl, hydroxyalkyl, alkoxycarbonyl, cycloalkyl, phenyl, heteroaryl, heterocyclyl and bicyclyl being independently unsubstituted or substituted by one or more Rc, and the alkyl being independently unsubstituted or substituted with one or more Rd;
each Rc is independently chosen from halogen atoms, hydroxyl, cyano, linear or branched C₁-C₆ alkyl, C₁-C₄ haloalkyl, C₁-C₄ alkoxy, C₁-C₄ hydroxyalkyl, C₃-C₇ cycloalkyl, phenyl, 5- to 6-membered monocyclic heteroaryl containing 1, 2 or 3 nitrogen atoms, 5- to 6-membered heterocyclyl containing 1, 2 or 3 nitrogen atoms, and C₃-C₇ heterocycloalkyl ketone containing 1, 2 or 3 nitrogen atoms, said phenyl being independently unsubstituted or substituted with one or more halogen atoms, and said heteroaryl, heterocyclyl and heterocycloalkyl ketone being independently unsubstituted or substituted by one or more linear or branched C₁-C₃ alkyl;
each Rd is independently chosen from cyano, C₁-C₄ haloalkyl, C₁-C₄ alkoxy, C₁-C₄ hydroxyalkyl, C₃-C₇ cycloalkyl, phenyl, 5- to 6-membered monocyclic heteroaryl containing 1, 2 or 3 nitrogen atoms, 5- to 6-membered heterocyclyl containing 1, 2 or 3 nitrogen atoms, and C₃-C₇ heterocycloalkyl ketone containing 1, 2 or 3 nitrogen atoms, said phenyl being independently unsubstituted or substituted with one or more halogen atoms, and said heteroaryl, heterocyclyl and heterocycloalkyl ketone being independently unsubstituted or substituted by one or more linear or branched C₁-C₃ alkyl;
R₁₃, R₁₄, and R₁₅ are each independently chosen from a hydrogen atom, cyano, linear or branched C₁-C₆ alkyl, C₁-C₄ haloalkyl, C₁-C₄ hydroxyalkyl, C₁-C₄ alkoxycarbonyl, C₃-C₇ cycloalkyl, monocyclic or polycyclic C₅-C₁₄ aryl, 5- to 14-membered heteroaryl containing at least one heteroatom selected from O, S and N, and 5- to 14-membered heterocyclyl containing at least one heteroatom selected from O, S and N, wherein the haloalkyl, hydroxyalkyl, alkoxycarbonyl, cycloalkyl, aryl, heteroaryl and heterocyclyl are independently unsubstituted or substituted by one or more Ra, and the alkyl groups are independently optionally substituted by one or more Rb.

3. The method according to claim 1, wherein
R₁, R₂ and R₄ are the same or different and each is independently chosen from a hydrogen atom, halogen atoms, hydroxyl, cyano, linear or branched C₁-C₆ alkyl, C₁-C₄ haloalkyl and C₁-C₄ hydroxyalkyl;
R₃ is chosen from a hydrogen atom, halogen atoms, hydroxyl, cyano, linear or branched C₁-C₆ alkyl, C₁-C₄ haloalkyl, C₁-C₄ hydroxyalkyl, C₃-C₁₀ cycloalkyl, 5- to 10-membered heterocyclyl, C₆-C₁₀ aryl, 5- to 10-membered heteroaryl, —C(O)OR′ and —C(O)NR″R″′, which cycloalkyl, heterocyclyl, aryl and heteroaryl are independently unsubstituted or substituted with one or more halogen atoms or linear or branched C₁-C₆ alkyl, hydroxyl, cyano, or C₁-C₄ alkoxy; wherein R′, R″ and R″′ are the same or different and each is independently chosen from a hydrogen atom, linear or branched C₁-C₆ alkyl, C₁-C₄ haloalkyl and C₁-C₄ hydroxyalkyl;
R₅ is chosen from a hydrogen atom, halogen atoms, hydroxyl, cyano, linear or branched C₁-C₆ alkyl, C₁-C₄ haloalkyl, C₁-C₄ hydroxyalkyl and —(C₁-C₄ alkyl)-(C₃-C₇ cycloalkyl), or R₅ together with R₈ and the nitrogen atom to which R₅ is bonded form a 5- to 9-membered, saturated heterocyclyl group, which contains, as heteroatoms, one or two nitrogen atoms and which heterocyclyl ring is independently unsubstituted or substituted with a 5- or 6-membered heteroaryl containing one or two nitrogen atoms, —C(O)—(CH₂)ₙ—R′ or —C(O)—(CH₂)ₙ—NR″R″′, wherein n is independently 0, 1 or 2, R′ is chosen from a hydrogen atom, linear or branched C₁-C₆ alkyl, C₁-C₄ haloalkyl, C₁-C₄ hydroxyalkyl, and cyano and R″ and R′″ are the same or different and each is independently chosen from a hydrogen atom, linear or branched C₁-C₆ alkyl, C₁-C₄ haloalkyl, and C₁-C₄ hydroxyalkyl;
R₆ and R₇ are the same or different and each is independently chosen from a hydrogen atom, linear or branched C₁-C₆ alkyl, C₁-C₄ haloalkyl, C₁-C₄ hydroxyalkyl and —(C₁-C₄ alkyl)-Het-(C₁-C₄ alkyl);
R₉ is a hydrogen atom, halogen atoms, hydroxyl, cyano, linear or branched C₁-C₆ alkyl, C₁-C₄ alkoxy, C₁-C₄ haloalkyl, C₁-C₄ hydroxyalkyl, C₃-C₇ cycloalkyl, 5- to 10-membered heterocyclyl, and 5- to 10-membered heteroaryl, which heterocyclyl and heteroaryl are independently unsubstituted or substituted with one or more halogen atoms, linear or branched C₁-C₆ alkyl, cyano, hydroxyl, carboxyl or C₁-C₄ alkoxy substituents, or R₉ is -Het-R′, Y′—R′″ or —C(O)—Het-R′, or when two adjacent —CR₉ groups are present, the two adjacent —CR₉ and the carbon atoms to which they are bonded optionally form a C₆-C₁₀ aryl group which is unsubstituted or substituted by one or more substituents selected from halogen atoms, linear or branched C₁-C₆ alkyl, hydroxyl and C₁-C₄ alkoxy;
R₈ together with R₅ and the nitrogen atom to which R₅ is bonded form a said 5- to 9-membered heterocyclyl ring, or R₈ is a hydrogen atom, linear or branched C₁-C₆ alkyl, C₁-C₄ haloalkyl, C₁-C₄ hydroxyalkyl, C₃-C₁₀ cycloalkyl, 5- to 10-membered heterocyclyl, 4- to 10-membered heterocycyl ring containing 1, 2 or 3 nitrogen atoms, which ring is substituted one, two or three oxo, C₆-C₁₀ aryl, 5- to 10-membered heteroaryl, -L-Het-R″′, -L-A, -A-SO₂—R′, -A-SO—R″′, -A-A′, -A-L-C(O)NR′R″, -A-L-CN, -A-C(O)—Het′-L-CN, -A-C(O)—NR′R″, -A-C(O)_{z}-A″, -A-C(O)—R″′, -A-CO₂—R′, -A-C(O)_{z}-L-A″′, -A-C(O)_{z}-L-CN, -A-C(O)-A′-A″, -A-C(O)-L-R′, -A-C(O)-L-CN, -A-Het-L-CN, -A-C(O)-L-Het-A′, -A-C(O)-L-Het-L-A′, -A-C(O)-L-Het-L-R″′, A-C(O)-L-Het-C(O)-A′, or -A-C(O)_{z}-L-Het-R′, wherein z is independently 1 or 2, R′ and R″ are the same or different and each is independently chosen from a hydrogen atom, C₁-C₄ haloalkyl, C₁-C₄ hydroxyalkyl, and linear or branched C₁-C₆ alkyl, which alkyl groups are independently unsubstituted or substituted by C₁-C₂ alkoxy or 5- or 6-membered heterocyclyl, and R″′ represents linear or branched C₁-C₆ alkyl, C₁-C₄ haloalkyl or C₁-C₄ hydroxyalkyl, the heterocyclyl and heteroaryl being optionally fused to a phenyl, and wherein the cycloalkyl, heterocyclyl, aryl and heteroaryl being independently unsubstituted or substituted with one or more halogen atoms, hydroxyl, cyano, C₁-C₄ alkoxy, or linear or branched C₁-C₄ alkyl, which alkyl is independently unsubstituted or substituted by cyano, and wherein
L is linear or branched C₁-C₆ alkylene, which is independently unsubstituted or substituted by one or two hydroxyl,
Het is O or NR, and Het′ is NR, wherein R is a hydrogen atom, straight or branched C₁-C₄ alkyl, C₁-C₄ haloalkyl, or C₁-C₄ hydroxyalkyl,
Y′ is —C(O)—, SO or SO₂,
A, A′, A″ and A″′ are the same or different and each is independently chosen from C₃-C₁₀ cycloalkyl, 5- to 10-membered heterocyclyl, 4- to 10-membered heterocycyl ring containing 1, 2 or 3 nitrogen atoms, which ring is substituted by one, two or three oxo, C₆-C₁₀ aryl, 5- to 10-membered heteroaryl and bicyclyl containing monocyclic C₅-C₆ aryl or heteroaryl bonded directly to 5- to 6-membered cycloalkyl or heterocyclyl, the cycloalkyl, heterocyclyl, aryl heteroaryl and bicyclyl being independently unsubstituted or substituted with one or more halogen atoms, hydroxyl, cyano, linear or branched C₁-C₄ alkyl, C₁-C₄ haloalkyl, C₁-C₄ hydroxyalkyl, or C₁-C₄ alkoxy.

4. The method according to claim 1, wherein Z is NR₅.

5. The method according to claim 1, wherein R₁ is chosen from a hydrogen atom, halogen atoms, hydroxyl, cyano, linear or branched C₁-C₆ alkyl, C₁-C₄ haloalkyl, C₁-C₄ hydroxyalkyl, C₁-C₄ alkoxy, C₁-C₄ alkoxycarbonyl, C₃-C₇ cycloalkyl, phenyl, pyridyl, 6-membered, saturated N-containing heterocyclyl ring, —C(O)OR₁₃, —C(O)—(CH₂)ₙ—R₁₃, —NR₁₃R₁₄, and —C(O)—(CH₂)ₙ—N₁₃R₁₄.

6. The method according to claim 1, wherein R₂ is chosen from a hydrogen atom, halogen atoms, hydroxyl, cyano, linear or branched C₁-C₆ alkyl, C₁-C₄ haloalkyl, C₁-C₄ alkoxy, C₁-C₄ hydroxyalkyl, C₁-C₄ alkoxycarbonyl, C₃-C₇ cycloalkyl, phenyl, pyridyl and 6 membered, saturated N-containing heterocyclyl ring.

7. The method according to claim 1, wherein R₃ is chosen from a hydrogen atom, halogen atoms, cyano, linear or branched C₁-C₆ alkyl, C₁-C₄ haloalkyl, C₁-C₄ hydroxyalkyl, C₁-C₄ alkoxy, C₁-C₄ alkoxycarbonyl, C₃-C₇ cycloalkyl, phenyl, 5- to 6-membered heteroaryl containing at least one heteroatom selected from O, S and N, and 6 membered, saturated N-containing heterocyclyl ring, the haloalkyl, hydroxyalkyl, alkoxycarbonyl, cycloalkyl, phenyl, heteroaryl and heterocyclyl being independently unsubstituted or substituted by one or more substituents selected from halogen atoms, hydroxyl, cyano or linear or branched C₁-C₆ alkyl, and the alkyl groups are independently unsubstituted or substituted by one or more cyano; or
R₃ is chosen from —S(O)₂NR₁₃R₁₄, —NR₁₃S(O)₂NR₁₄, —OR₁₃, —C(O)OR₁₃, —NR₁₃R₁₄, —NC(O)—(CH₂)ₙ—R₁₃, —C(O)—(CH₂)ₙ—NR₁₃R₁₄, —NR₁₃C(O)—(CH₂)ₙ—R₁₄ and —NR₁₃C(O)—(CH₂)ₙ—NR₁₄R₁₅, wherein n is 0 or 1, and R₁₃, R₁₄, and R₁₅ are each independently chosen from a hydrogen atom, cyano, linear or branched C₁-C₆ alkyl, C₁-C₄ haloalkyl, C₁-C₄ hydroxyalkyl, C₁-C₄ alkoxycarbonyl, C₃-C₇ cycloalkyl, phenyl and 6-membered, saturated N-containing heterocyclyl ring.

8. The method according to claim 7, wherein R₃ is chosen from a hydrogen atom, halogen atoms, cyano, C₁-C₄ haloalkyl, C₃-C₄ cycloalkyl, phenyl, 5- to 6-membered monocyclic heteroaryl containing 1, 2 or 3 heteroatoms selected from N, O and S, the phenyl and heteroaryl being independently unsubstituted or substituted with one or more substituents selected from halogen atoms, hydroxyl, cyano and linear or branched C₁-C₆ alkyl; or
R₃ is —C(O)OR₁₃ or —C(O)—(CH₂)ₙ—NR₁₃R₁₄ wherein R₁₃ and R₁₄ are each independently chosen from a hydrogen atom, cyano, linear or branched C₁-C₆ alkyl, C₁-C₄ haloalkyl, C₁-C₄ hydroxyalkyl, C₁-C₄ alkoxycarbonyl, C₃-C₇ cycloalkyl, phenyl and 6 membered, saturated N-containing heterocyclyl ring.

9. The method according to claim 1, wherein R₄ is chosen from a hydrogen atom, halogen atoms, hydroxyl, cyano, linear or branched C₁-C₆ alkyl, C₁-C₄ haloalkyl, C₁-C₄ hydroxyalkyl, C₁-C₄ alkoxy, C₁-C₄ alkoxycarbonyl, C₃-C₇ cycloalkyl, phenyl, pyridyl and 6 membered, saturated N-containing heterocyclyl ring.

10. The method according to claim 1, wherein R₅ is a hydrogen atom, or linear or branched C₁-C₆ alkyl optionally substituted by one or more substituents selected from hydroxyl, cyano, linear or branched C₁-C₆ alkyl, C₁-C₄ haloalkyl, C₁-C₄ hydroxyalkyl, C₃-C₇ cycloalkyl, phenyl and 6 membered, saturated N-containing heterocyclyl ring.

11. The method according to claim 1, wherein R₆ and R₇ are each independently a hydrogen atom or linear or branched C₁-C₆ alkyl unsubstituted or substituted by one or more C₁-C₂ alkoxy.

12. The method according to claim 1, wherein R₆ and R₇ are each independently a hydrogen atom or linear or branched C₁-C₆ alkyl.

13. The method according to claim 1, wherein R₈ is chosen from linear or branched C₁-C₆ alkyl, C₁-C₄ haloalkyl, —(C₁-C₆ alkyl)-(C₁-C₄ alkoxy), C₃-C₁₀ cycloalkyl, phenyl, naphthyl, 5- to 6-membered monocyclic heteroaryl containing 1, 2 or 3 heteroatoms selected from N, O and S, 5- to 7-membered heterocyclyl containing 1, 2 or 3 heteroatoms selected from N, O and S, C₃-C₇ heterocycloalkyl ketone containing 1, 2 or 3 nitrogen atoms, and bicyclyl containing monocyclic C₅-C₉ aryl and heteroaryl bonded directly to 5- to 9-membered cycloalkyl or heterocyclyl, said heteroaryl or heterocyclyl containing at least one heteroatom selected from O, S and N,
the cycloalkyl, phenyl, naphthyl, heteroaryl, heterocyclyl, heterocycloalkyl ketone and bicyclyl are independently unsubstituted or substituted by one or more substituents selected from halogen atoms; hydroxyl; cyano; linear or branched C₁-C₆ alkyl; C₁-C₄ haloalkyl; C₃-C₇ cycloalkyl; phenyl, which phenyl is unsubstituted or substituted by one or more substituents selected from halogen atoms or cyano; 5- to 6-membered monocyclic heteroaryl containing 1, 2 or 3 heteroatoms selected from N, O and S, which heteroaryl is independently unsubstituted or substituted by one or more substituents selected from halogen atoms or cyano; 6 membered, saturated N-containing heterocyclyl ring; —S(O)₂R₁₀; —S(O)₂NR₁₀R₁₁; —NR₁₀S(O)₂NR₁₁; —OR₁₀; —C(O)OR₁₀; —C(O)—CH₂—OR₁₀; —C(O)—(CH₂)ₙ—O—(CH₂)ₚR₁₀; —C(O)—(CH₂)ₙ—O—(CH₂)ₚ—O—R₁₀; —C(O)—(CH₂)ₙ—R₁₀; —NR₁₀R₁₁; —C(O)—(CH₂)ₙ—NR₁₀R₁₁; —R₁₀C(O)—(CH₂)ₙ—NR₁₁R₁₂; —C(C₁-C₄ alkyl)₂-C(O)—(CH₂)ₙ—NR₁₀R₁₁; —(C₁-C₄ alkyl)-CN, and —(C₁-C₄ alkyl)-C(O)NR′R″ wherein R′ and R″ are the same or different and are independently selected from hydrogen atoms and linear or branched C₁-C₄ alkyl s; wherein each n is independently 0 or an integer from 1 to 5, and wherein each p is an integer from 1 to 3, and wherein
R₁₀, R₁₁ and R₁₂ are each independently chosen from a hydrogen atom; cyano; linear or branched C₁-C₆ alkyl; C₁-C₄ haloalkyl; C₁-C₆ hydroxyalkyl; C₃-C₇ cycloalkyl; phenyl; —(C₁-C₄ hydroxyalkyl)-(phenyl); —C(O)-(phenyl); 5- to 6-membered monocyclic heteroaryl containing 1, 2, 3 or 4 heteroatoms selected from N, O and S; 5- to 6-membered monocyclic heteroaryl ketone containing 1, 2 or 3 nitrogen atoms and 1 or 2 ketone groups; 5- to 6-membered, heterocyclyl containing 1, 2 or 3 heteroatoms selected from N, O and S; and bicyclyl containing a monocyclic C₅-C₆ aryl or heteroaryl bonded directly to a 5- to 6-membered cycloalkyl or heterocyclyl, said heteroaryl or heterocyclyl containing 1, 2 or 3 heteroatoms selected from N, O and S,
the cycloalkyl, phenyl, heteroaryl, heteroaryl ketone, heterocyclyl and bicyclyl being independently unsubstituted or substituted by one or more substituents selected from halogen atoms, hydroxyl, cyano, linear or branched C₁-C₆ alkyl, C₁-C₄ haloalkyl, C₁-C₄ hydroxyalkyl, C₁-C₄ alkoxy, phenyl independently unsubstituted or substituted by one or more halogen atoms, 5- to 6-membered monocyclic heteroaryl containing 1, 2 or 3 nitrogen atoms, said heteroaryl being unsubstituted or substituted by one or more linear or branched C₁-C₃ alkyl, or C₃-C₇ heterocycloalkyl ketone containing 1, 2 or 3 nitrogen atoms, said heterocycloalkyl ketone being unsubstituted or substituted by one or more C₁-C₃ alkyl; or
R₈ is chosen from —S(O)₂NR₁₃R₁₄, —NR₁₃S(O)₂NR₁₄, —OR₁₃, —C(O)OR₁₃ and —C(O)—(CH₂)ₙ—NR₁₃R₁₄, wherein each n is independently 0 or 1, and each R₁₃ and R₁₄ is independently chosen from a hydrogen atom; cyano; linear or branched C₁-C₆ alkyl, which alkyl groups are independently unsubstituted or substituted by one or more phenyl; C₁-C₄ haloalkyl; C₁-C₄ hydroxyalkyl; C₁-C₄ alkoxycarbonyl; C₃-C₇ cycloalkyl; phenyl or 6 membered, saturated N-containing heterocyclyl ring; or
R₈ together with R₅ and the nitrogen atom to which R₅ is bonded form a 5- to 9-membered, saturated heterocyclyl group, which contains, as heteroatoms, one or two nitrogen atoms and which is substituted by 5- to 6-membered monocyclic heteroaryl containing 1, 2 or 3 heteroatoms selected from N, O and S; —C(O)—(CH₂)ₙ—R₁₀; or —C(O)—(CH₂)ₙ—NR₁₀R₁₁, wherein each n is independently 0 or 1, and each R₁₀ and R₁₁ are independently chosen from a hydrogen atom, cyano, linear or branched C₁-C₆ alkyl, C₁-C₄ haloalkyl, C₁-C₄ hydroxyalkyl, C₁-C₄ alkoxycarbonyl, C₃-C₇ cycloalkyl, phenyl and 6 membered, saturated N-containing heterocyclyl ring,
provided that when m is zero, R₈ is other than —S(O)₂NR₁₃R₁₄, —NR₁₃S(O)₂NR₁₄, or —OR₁₃.

14. The method according to claim 13, wherein R₈ is chosen from linear or branched C₁-C₆ alkyl, C₁-C₄ haloalkyl, C₃-C₁₀ cycloalkyl, phenyl, nphthyl, 5- to 6-membered monocyclic heteroaryl containing 1, 2 or 3 heteroatoms selected from N, O and S, 5- to 7-membered heterocyclyl containing 1, 2 or 3 heteroatoms selected from N, O and S, and bicyclyl containing monocyclic C₅-C₉ aryl and heteroaryl bonded directly to 5- to 9-membered cycloalkyl or heterocyclyl, said heteroaryl or heterocyclyl containing at least one heteroatom selected from O, S and N,
the alkyl, haloalkyl, alkoxy, cycloalkyl, phenyl, naphthyl, heteroaryl, heterocyclyl and bicyclyl being independently unsubstituted or substituted by one or more substituents selected from halogen atoms, hydroxyl, cyano, linear or branched C₁-C₆ alkyl, C₁-C₄ haloalkyl, C₃-C₇ cycloalkyl, phenyl unsubstituted or substituted by one or more halogen atoms, 5- to 6-membered monocyclic heteroaryl containing 1, 2 or 3 heteroatoms selected from N, O and S, 6 membered, saturated N-containing heterocyclyl ring, —S(O)₂R₁₀, —S(O)₂NR₁₀R₁₁, —NR₁₀S(O)₂NR₁₁, —OR₁₀, —C(O)OR₁₀, —C(O)—(CH₂)ₙ—R₁₀, —NR₁₀R₁₁, —C(O)—(CH₂)ₙ—NR₁₀R₁₁ —R₁₀C(O)—(CH₂)ₙ—NR₁₁R₁₂, —(C₁-C₄ alkyl)-CN, and —(C₁-C₄ alkyl)-C(O)NR′R″ wherein R′ and R″ are the same or different and are independently selected from hydrogen atoms and linear or branched C₁-C₄ alkyl; wherein n is 0 or 1, and
R₁₀ and R₁₁ are each independently chosen from a hydrogen atom, cyano, linear or branched C₁-C₆ alkyl, C₁-C₄ haloalkyl, C₃-C₇ cycloalkyl, phenyl, 5- to 6-membered monocyclic heteroaryl containing 1, 2 or 3 heteroatoms selected from N, O and S, 5- to 6-membered, heterocyclyl containing 1, 2 or 3 nitrogen atoms, and bicyclyl containing a monocyclic C₅-C₆ aryl or heteroaryl bonded directly to a 5- to 6-membered cycloalkyl or heterocyclyl, said heteroaryl or heterocyclyl containing 1, 2 or 3 nitrogen atoms,
the alkyl, haloalkyl, cycloalkyl, phenyl, heteroaryl, heterocyclyl and bicyclyl being independently unsubstituted or substituted by one or more substituents selected from halogen atoms, hydroxyl, cyano, linear or branched C₁-C₆ alkyl, C₁-C₄ haloalkyl, phenyl independently unsubstituted or substituted by one or more halogen atoms, 5- to 6-membered monocyclic heteroaryl containing 1, 2 or 3 nitrogen atoms, said heteroaryl being unsubstituted or substituted by one or more linear or branched C₁-C₃ alkyl, and C₃-C₇ heterocycloalkyl ketone containing 1, 2 or 3 nitrogen atoms, said heterocycloalkyl ketone being unsubstituted or substituted by one or more C₁-C₃ alkyl; or
R₈ is chosen from —S(O)₂NR₁₃R₁₄, —NR₁₃S(O)₂NR₁₄, —OR₁₃, —C(O)OR₁₃ and —C(O)—(CH₂)ₙ—NR₁₃R₁₄, wherein n is 0 or 1, and R₁₃ and R₁₄ are each independently chosen from a hydrogen atom, cyano, linear or branched C₁-C₆ alkyl unsubstituted or substituted by one or more phenyl, C₁-C₄ haloalkyl, C₁-C₄ hydroxyalkyl, C₁-C₄ alkoxycarbonyl, C₃-C₇ cycloalkyl, phenyl and 6 membered, saturated N-containing heterocyclyl ring; or
R₈ together with R₅ and the nitrogen atom to which R₅ is bonded form a 5- to 9-membered, saturated heterocyclyl group, which contains, as heteroatoms, one or two nitrogen atoms and which is substituted by —C(O)—(CH₂)ₙ—R₁₀, or —C(O)—(CH₂)ₙ—NR₁₀R₁₁, wherein n is independently 0 or 1, and R₁₀ and R₁₁ are each independently chosen from a hydrogen atom, cyano, linear or branched C₁-C₆ alkyl, C₁-C₄ haloalkyl, C₁-C₄ hydroxyalkyl, C₁-C₄ alkoxycarbonyl, C₃-C₇ cycloalkyl, phenyl and 6 membered, saturated N-containing heterocyclyl ring,
provided that when m is zero, R₈ is other than —S(O)₂NR₁₃R₁₄, —NR₁₃S(O)₂NR₁₄, or —OR₁₃.

15. The method according to claim 13, wherein R₈ is chosen from linear or branched C₁-C₆ alkyl, C₁-C₄ haloalkyl, C₁-C₄ alkoxy, C₃-C₇ cycloalkyl, adamantyl, phenyl, 5- to 6-membered monocyclic heteroaryl containing 1, 2 or 3 heteroatoms selected from N, O and S, 5- to 7-membered heterocyclyl containing 1, 2 or 3 heteroatoms selected from N, O and S, and bicyclyl containing a phenyl bonded directly to a 5- to 7-membered heterocyclyl containing at least one heteroatom selected from O, S and N,
the alkyl, haloalkyl, alkoxy, cycloalkyl, adamantyl, phenyl, heteroaryl, heterocyclyl and bicyclyl being independently unsubstituted or substituted by one or more substituents selected from halogen atoms, hydroxyl, linear or branched C₁-C₆ alkyl, phenyl, pyridyl, —S(O)₂R₁₀, —C(O)OR₁₀, —C(O)—(CH₂)ₙ—R₁₀, —NR₁₀R₁₁, —C(O)—(CH₂)ₙ—NR₁₀R₁₁, —(C₁-C₄ alkyl)-CN, and —(C₁-C₄ alkyl)-C(O)NR′R″ wherein R′ and R″ are the same or different and are selected from hydrogen atoms and linear or branched C₁-C₄ alkyl groups; wherein n is 0 or 1, and
R₁₀ and R₁₁ are each independently chosen from a hydrogen atom, cyano, linear or branched C₁-C₆ alkyl, C₁-C₄ haloalkyl, C₃-C₇ cycloalkyl, phenyl, 5- to 6-membered monocyclic heteroaryl containing 1, 2 or 3 heteroatoms selected from N, O and S, 5- to 6-membered, heterocyclyl containing 1, 2 or 3 nitrogen atoms, and bicyclyl containing a monocyclic C₅-C₆ aryl or heteroaryl bonded directly to a 5- to 6-membered cycloalkyl or heterocyclyl, said heteroaryl or heterocyclyl containing 1, 2 or 3 nitrogen atoms,
the alkyl, haloalkyl, cycloalkyl, phenyl, heteroaryl, heterocyclyl and bicyclyl being independently unsubstituted or substituted by one or more substituents selected from halogen atoms, hydroxyl, cyano, linear or branched C₁-C₆ alkyl, C₁-C₄ haloalkyl, phenyl unsubstituted or substituted by one or more halogen atoms, 5- to 6-membered monocyclic heteroaryl containing 1, 2 or 3 nitrogen atoms, said heteroaryl being unsubstituted or substituted by one or more linear or branched C₁-C₃ alkyl, and C₃-C₇ heterocycloalkyl ketone containing 1, 2 or 3 nitrogen atoms, said heterocycloalkyl ketone being unsubstituted or substituted by one or more linear or branched C₁-C₃ alkyl; or
R₈ is —OR₁₃ wherein R₁₃ is C₁-C₃ alkyl unsubstituted or substituted by one or more phenyl, or
R₈ together with R₅ and the nitrogen atom to which R₅ is bonded form a 5- to 7-membered, saturated heterocyclyl group, which contains, as heteroatoms, one or two nitrogen atoms and which is substituted by —C(O)—(CH₂)ₙ—R₁₀ or —C(O)—(CH₂)ₙ—NR₁₀R₁₁, wherein n is 0 or 1, and R₁₀ and R₁₁ are each independently chosen from a hydrogen atom, cyano, linear or branched C₁-C₃ alkyl and C₁-C₄ haloalkyl,
provided that when m is zero, R₈ is other than an —OR₁₃.

16. The method according to claim 1, wherein R₉ is chosen from a hydrogen atom, halogen atoms, hydroxyl, cyano, linear or branched C₁-C₆ alkyl, C₁-C₄ haloalkyl, C₁-C₄ alkoxy, C₃-C₇ cycloalkyl, —C₁-C₄ alkyl-C₃-C₇ cycloalkyl, phenyl, 5- to 6-membered monocyclic heteroaryl containing 1, 2, 3 or 4 heteroatoms selected from N, O and S, and 5- to 7-membered heterocyclyl containing at least one heteroatom selected from O, S and N,
wherein the cycloalkyl, phenyl, heteroaryl and heterocyclyl are independently unsubstituted or substituted by one or more substituents selected from halogen atoms, hydroxyl, cyano, linear or branched C₁-C₆ alkyl, —S(O)₂R₁₀, —C(O)OR₁₀, —C(O)—(CH₂)—R₁₀ and —OR₁₀,
wherein R₁₀ is chosen from a hydrogen atom, cyano, linear or branched C₁-C₄ alkyl, C₁-C₄ haloalkyl and C₃-C₇ cycloalkyl,
wherein the cycloalkyl groups are independently unsubstituted or substituted by one or more substituents selected from halogen atoms, hydroxyl, cyano, linear or branched C₁-C₆ alkyl and C₁-C₄ haloalkyl; or
R₉ is chosen from —S(O)₂R₁₃, —OR₁₃, —C(O)OR₁₃, —NR₁₃R₁₄ and —C(O)—(CH₂)ₙ—NR₁₃R₁₄ wherein n is 0 or 1 and R₁₃ and R₁₄ are each independently chosen from a hydrogen atom, or linear or branched C₁-C₆ alkyl, —(C₁-C₄ alkyl)-(C₁-C₄ alkoxy), and —(C₁-C₄ alkyl)-(5- to 7-membered heterocyclyl containing at least one heteroatom selected from O, S and N); or
when two —CR₉ adjacent groups are present, two adjacent —CR₉ grows and the carbon atoms to which they are bonded optionally form a 5- to 9-membered aryl or heteroaryl group, said heteroaryl containing at least one heteroatom selected from O, S and N, wherein the aryl and heteroaryl are independently unsubstituted or substituted by one or more substituents selected from halogen atoms and linear or branched C₁-C₄ alkyl.

17. The method according to claim 16, wherein R₉ is chosen from a hydrogen atom, halogen atoms, hydroxyl, cyano, linear or branched C₁-C₆ alkyl, C₁-C₄ haloalkyl, C₁-C₄ alkoxy, C₃-C₇ cycloalkyl, —C₁-C₄ alkyl-C₃-C₇ cycloalkyl, phenyl, 5- to 6-membered monocyclic heteroaryl containing 1, 2 or 3 heteroatoms selected from N, O and S, and 5- to 7-membered heterocyclyl containing at least one heteroatom selected from O, S and N, the alkyl, haloalkyl, alkoxy, cycloalkyl, phenyl, heteroaryl and heterocyclyl being independently unsubstituted or substituted by one or more substituents selected from halogen atoms, hydroxyl, cyano, linear or branched C₁-C₆ alkyl, —C(O)—(CH₂)—R₁₀ and —OR₁₀,
wherein R₁₀ is chosen from a hydrogen atom, cyano, linear or branched C₁-C₄ alkyl, C₁-C₄ haloalkyl and C₃-C₇ cycloalkyl, the alkyl, haloalkyl and cycloalkyl being independently unsubstituted or substituted by one or more substituents selected from halogen atoms, hydroxyl, cyano, linear or branched C₁-C₆ alkyl and C₁-C₄ haloalkyl; or
R₉ is chosen from —S(O)₂R₁₃, —C(O)OR₁₃, —NR₁₃R₁₄ and —C(O)—(CH₂)ₙ—NR₁₃R₁₄ wherein n is 0 or 1 and R₁₃ and R₁₄ are each independently a hydrogen atom or linear or branched C₁-C₆ alkyl; or when two —CR₉ adjacent groups are present, two adjacent —CR₉ groups and the carbon atoms to which they are bonded optionally form a 5- to 9-membered aryl or heteroaryl, said heteroaryl containing at least one heteroatom selected from O, S and N, the aryl and heteroaryl being unsubstituted or substituted by one or more substituents selected from halogen atoms and linear or branched C₁-C₄ alkyl.

18. The method according to claim 16, wherein R₉ is chosen from a hydrogen atom, halogen atoms, cyano, linear or branched C₁-C₄ alkyl, C₁-C₄ haloalkyl, C₃-C₇ cycloalkyl, C₁-C₃ alkyl —C₃-C₇ cycloalkyl, 5- to 6-membered monocyclic heteroaryl containing 1, 2 or 3 nitrogen atoms, and 5- to 7-membered heterocyclyl containing 1, 2 or 3 nitrogen atoms, the alkyl, cycloalkyl, heteroaryl and heterocyclyl being independently unsubstituted or substituted by one or more substituents selected from cyano and —OR₁₀
wherein R₁₀ is a hydrogen atom or linear or branched C₁-C₃ alkyl, or
R₉ is chosen from —S(O)₂R₁₃, —C(O)OR₁₃, —NR₁₃R₁₄ and —C(O)—(CH₂)ₙ—NR₁₃R₁₄ wherein n is 0 or 1 and R₁₃ and R₁₄ are each independently a hydrogen atom or linear or branched C₁-C₆ alkyl.

19. The method according to claim 1, wherein
m is 0 or 1;
Z is NR₅;
X is a nitrogen atom and Y is —CR₉, or Y is a nitrogen atom and X is —CR₉;
R₁ is chosen from a hydrogen atom, halogen atoms, cyano, linear or branched C₁-C₆ alkyl, C₃-C₄ cycloalkyl and —NR₁₃N₁₄ wherein R₁₃ and R₁₄ are each independently a hydrogen atom or linear or branched C₁-C₃ alkyl;
R₂ is chosen from a hydrogen atom, halogen atoms, cyano, linear or branched C₁-C₆ alkyl and C₃-C₄ cycloalkyl;
R₃ is chosen from a hydrogen atom, halogen atoms, cyano, C₃-C₄ cycloalkyl, 5- to 6-membered monocyclic aryl or heteroaryl, the heteroaryl containing 1, 2 or 3 heteroatoms selected from N, O and S, the aryl and heteroaryl being independently unsubstituted or substituted by one or more substituents selected from halogen atoms, hydroxyl, cyano and linear or branched C₁-C₆ alkyl, or
R₃ is chosen from —C(O)OR₁₃, —C(O)—NR₁₃R₁₄ and —NR₁₃C(O)R₁₄ wherein R₁₃ and R₁₄ are each independently chosen from a hydrogen atom, linear or branched C₁-C₆ alkyl, C₁-C₄ haloalkyl, C₁-C₄ hydroxyalkyl, C₁-C₄ alkoxycarbonyl, C₃-C₇ cycloalkyl, phenyl and 6 membered, saturated N-containing heterocyclyl ring;
R₄ is chosen from a hydrogen atom, halogen atoms, cyano, linear or branched C₁-C₆ alkyl and C₃-C₄ cycloalkyl;
R₅ is chosen from a hydrogen atom, linear or branched C₁-C₃ alkyl, and C₁-C₃ alkyl-C₃-C₇ cycloalkyl;
R₆ and R₇ are each independently chosen from a hydrogen atom or linear or branched C₁-C₃ alkyl unsubstituted or substituted by C₁-C₂ alkoxy;
R₈ is chosen from linear or branched C₁-C₆ alkyl, C₁-C₄ haloalkyl, C₃-C₇ cycloalkyl, adamantyl, phenyl, 5- to 6-membered monocyclic heteroaryl containing 1, 2 or 3 heteroatoms selected from N, O and S, 5- to 7-membered heterocyclyl containing 1, 2 or 3 heteroatoms selected from N, O and S, C₃-C₇ heterocycloalkyl ketone containing 1, 2 or 3 nitrogen atoms, bicyclyl containing a phenyl bonded directly to a 5- to 7-membered heterocyclyl containing at least one heteroatom selected from O, S and N, and bicyclyl containing a pyridinyl bonded directly to a C₃-C₇ cycloalkyl, wherein the cycloalkyl, adamantyl, phenyl, heteroaryl, heterocyclyl, heterocycloalkyl ketone and bicyclyl are independently unsubstituted or substituted by one or more substituents selected from halogen atoms; hydroxyl; linear or branched C₁-C₆ alkyl; phenyl, which phenyl is independently unsubstituted or substituted by one or more substituents selected from halogen atoms or cyano; 5- to 6-membered monocyclic heteroaryl containing 1, 2 or 3 heteroatoms selected from N, O and S, which heteroaryl is unsubstituted or substituted by one or more substituents selected from halogen atoms or cyano, —S(O)₂R₁₀, —C(O)OR₁₀, —C(O)—CH₂—OR₁₀, —C(O)—(CH₂)ₙ—O—(CH₂)ₚR₁₀, —C(O)—(CH₂)ₙ—O—(CH₂)ₚ—O—R₁₀, —C(O)—(CH₂)ₙ—R₁₀, —NR₁₀R₁₁, —C(O)—(CH₂)ₙ—NR₁₀R₁₁, —C(C₁-C₄ alkyl)₂-C(O)—(CH₂)ₙ—NR₁₀R₁₁, —(C₁-C₄ alkyl)-CN, and —(C₁-C₄ alkyl)-C(O)NR′R″ wherein R′ and R″ are the same or different and are independently selected from hydrogen atoms and linear or branched C₁-C₄ alkyl; wherein each n is independently 0 or an integer from 1 to 5, and wherein p is an integer from 1 to 3, and wherein
R₁₀ and R₁₁ are each independently chosen from a hydrogen atom, cyano, linear or branched C₁-C₆ alkyl, C₁-C₄ haloalkyl, C₁-C₆ hydroxyalkyl, C₃-C₇ cycloalkyl, phenyl, —(C₁-C₄ hydroxyalkyl)-(phenyl), —C(O)-(phenyl), 5- to 6-membered monocyclic heteroaryl containing 1, 2, 3 or 4 heteroatoms selected from N, O and S, 9- to 10-membered bicyclic heteroaryl containing 1, 2 or 3 nitrogen atoms, 5- to 6-membered monocyclic heteroaryl ketone containing 1, 2 or 3 nitrogen atoms and 1 or 2 ketone groups, 5- to 6-membered, heterocyclyl containing 1, 2 or 3 heteroatoms selected from N, O and S, and bicyclyl containing a monocyclic C₅-C₆ aryl or heteroaryl bonded directly to a 5- to 6-membered cycloalkyl or heterocyclyl, said heteroaryl or heterocyclyl containing 1, 2 or 3 heteroatoms selected from N, O and S,
the cycloalkyl, phenyl, heteroaryl, heterocyclyl and bicyclyl being independently unsubstituted or substituted by one or more substituents selected from halogen atoms; hydroxy; cyano; linear or branched C₁-C₆ alkyl; C₁-C₄ haloalkyl; C₁-C₄ hydroxyalkyl; C₁-C₄ alkoxy; phenyl, which phenyl is unsubstituted or substituted by one or more halogen atoms; 5- to 6-membered monocyclic heteroaryl containing 1, 2 or 3 nitrogen atoms, which heteroaryl is unsubstituted or substituted by one or more linear or branched C₁-C₃ alkyl; and C₃-C₇ heterocycloalkyl ketone containing 1, 2 or 3 nitrogen atoms, which heterocycloalkyl ketone is unsubstituted or substituted by one or more linear or branched C₁-C₃ alkyl; or
R₈ is —OR₁₃ wherein R₁₃ represents C₁-C₃ alkyl unsubstituted or substituted by one or more phenyl, or
R₈ together with R₅ and the nitrogen atom to which R₅ is bonded form a 5- to 7-membered, saturated heterocyclyl group, which contains, as heteroatoms, one or two nitrogen atoms and which is substituted by pyridinyl, —C(O)—(CH₂)ₙ—R₁₀ or —C(O)—(CH₂)ₙ—NR₁₀R₁₁, wherein each n is independently 0 or 1, and wherein each R₁₀ and R₁₁ are independently chosen from a hydrogen atom, cyano, linear or branched C₁-C₃ alkyl and C₁-C₄ haloalkyl,
R₉ is chosen from a hydrogen atom, halogen atoms, cyano, linear or branched C₁-C₄ alkyl, C₁-C₄ haloalkyl, C₃-C₇ cycloalkyl, 5- to 6-membered monocyclic heteroaryl containing 1, 2, 3 or 4 heteroatoms selected from N, O and S, and 5- to 7-membered heterocyclyl containing 1, 2 or 3 heteroatoms selected from N, O and S, wherein the alkyl, cycloalkyl, heteroaryl, and heterocyclyl are independently unsubstituted or substituted by one or more substituents selected from halogen atoms, cyano, hydroxyl, linear or branched C₁-C₆ alkyl, —S(O)₂R₁₀, —C(O)OR₁₀, —C(O)—(CH₂)—R₁₀ and —OR₁₀, wherein R₁₀ is chosen from a hydrogen atom, cyano and linear or branched C₁-C₃ alkyl, or
R₉ is chosen from —S(O)₂R₁₃, —OR₁₃, —C(O)OR₁₃, —NR₁₃R₁₄ and —C(O)—(CH₂)ₙ—NR₁₃R₁₄ wherein n is 0 or 1 and R₁₃ and R₁₄ are each independently chosen from a hydrogen atom, linear or branched C₁-C₆ alkyl, —(C₁-C₄ alkyl)-(C₁-C₄ alkoxy), and —(C₁-C₄ alkyl)-(5- to 7-membered heterocyclyl containing at least one heteroatom selected from O, S and N); or when two adjacent —CR₉ adjacent groups are present, both —CR₉ and the carbon atoms to which they are bonded optionally form a 5- to 6-membered aryl group, wherein the aryl is unsubstituted or substituted by one or more substituents selected from halogen atoms and linear or branched C₁-C₄ alkyl,
provided that when m is zero, R₈ is other than an —OR₁₃.

20. The method according to claim 1, wherein:
m is 0 or 1;
Z is NR₅;
X is a nitrogen atom and Y is —CR₉, or Y is a nitrogen atom and X is —CR₉;
R₁ is chosen from a hydrogen atom, halogen atoms, cyano, linear or branched C₁-C₆ alkyl, C₃-C₄ cycloalkyl and —NR₁₃N₁₄ wherein R₁₃ and R₁₄ are each independently a hydrogen atom or linear or branched C₁-C₃ alkyl;
R₂ is chosen from a hydrogen atom, halogen atoms, cyano, linear or branched C₁-C₆ alkyl or C₃-C₄ cycloalkyl;
R₃ is chosen from a hydrogen atom, halogen atoms, cyano, C₃-C₄ cycloalkyl, 5- to 6-membered monocyclic aryl or heteroaryl, the heteroaryl containing 1, 2 or 3 heteroatoms selected from N, O and S, the aryl and heteroaryl being unsubstituted or substituted by one or more substituents selected from halogen atoms, hydroxyl, cyano and linear or branched C₁-C₆ alkyl, or R₃ is chosen from —C(O)OR₁₃, —C(O)—NR₁₃R₁₄ and —NR₁₃C(O)R₁₄ wherein R₁₃ and R₁₄ are each independently chosen from a hydrogen atom, linear or branched C₁-C₆ alkyl, C₁-C₄ haloalkyl, C₁-C₄ hydroxyalkyl, C₁-C₄ alkoxycarbonyl, C₃-C₇ cycloalkyl, phenyl and 6 membered, saturated N-containing heterocyclyl ring;
R₄ is chosen from a hydrogen atom, halogen atoms, cyano, linear or branched C₁-C₆ alkyl and C₃-C₄ cycloalkyl;
R₅ is chosen from a hydrogen atom, linear or branched C₁-C₃ alkyl and C₁-C₃ alkyl-C₃-C₇ cycloalkyl;
R₆ and R₇ each independently represent a hydrogen atom or linear or branched C₁-C₃ alkyl;
R₈ is chosen from linear or branched C₁-C₆ alkyl, C₁-C₄ haloalkyl, C₁-C₄ alkoxy, C₃-C₇ cycloalkyl, adamantyl, phenyl, 5- to 6-membered monocyclic heteroaryl containing 1, 2 or 3 heteroatoms selected from N, O and S, 5- to 7-membered heterocyclyl containing 1, 2 or 3 heteroatoms selected from N, O and S, and bicyclyl containing a phenyl bonded directly to a 5- to 7-membered heterocyclyl group containing at least one heteroatom selected from O, S and N, the alkyl, haloalkyl, alkoxy, cycloalkyl, adamantyl, phenyl, heteroaryl, heterocyclyl and bicyclyl being independently unsubstituted or substituted by one or more substituents selected from halogen atoms, hydroxyl, linear or branched C₁-C₆ alkyl, phenyl, pyridyl, —S(O)₂R₁₀, —C(O)OR₁₀, —C(O)—(CH₂)ₙ—R₁₀, —NR₁₀R₁₁, —C(O)—(CH₂)ₙ—NR₁₀R₁₁, —(C₁-C₄ alkyl)-CN, and —(C₁-C₄ alkyl)-C(O)NR′R″ wherein R′ and R″ are the same or different and are selected from hydrogen atoms and linear or branched C₁-C₄ alkyl s; wherein n is 0 or 1, and
R₁₀ and R₁₁ are each independently chosen from a hydrogen atom, cyano, linear or branched C₁-C₆ alkyl, C₁-C₄ haloalkyl, C₃-C₇ cycloalkyl, phenyl, 5- to 6-membered monocyclic heteroaryl containing 1, 2 or 3 heteroatoms selected from N, O and S, 5- to 6-membered heterocyclyl containing 1, 2 or 3 nitrogen atoms, and bicyclyl containing a monocyclic C₅-C₆ aryl or heteroaryl bonded directly to a 5- to 6-membered cycloalkyl or heterocyclyl, said heteroaryl or heterocyclyl containing 1, 2 or 3 nitrogen atoms,
the alkyl, haloalkyl, cycloalkyl, phenyl, heteroaryl, heterocyclyl and bicyclyl being independently unsubstituted or substituted by one or more substituents selected from halogen atoms, hydroxyl, cyano, linear or branched C₁-C₆ alkyl, C₁-C₄ haloalkyl, phenyl unsubstituted or substituted by one or more halogen atoms, 5- to 6-membered monocyclic heteroaryl containing 1, 2 or 3 nitrogen atoms, said heteroaryl unsubstituted or substituted by one or more linear or branched C₁-C₃ alkyl, or C₃-C₇ heterocycloalkyl ketone containing 1, 2 or 3 nitrogen atoms, said heterocycloalkyl ketone being unsubstituted or substituted by one or more linear or branched C₁-C₃ alkyl, or
R₈ is —OR₁₃ wherein R₁₃ is C₁-C₃ alkyl unsubstituted or substituted by one or more phenyl, or
R₈ together with R₅ and the nitrogen atom to which R₅ is bonded form a 5- to 7-membered, saturated heterocyclyl group, which contains, as heteroatoms, one or two nitrogen atoms and which is substituted by —C(O)—(CH₂)ₙ—R₁₀ or —C(O)—(CH₂)ₙ—NR₁₀R₁₁, wherein n is independently 0 or 1, and R₁₀ and R₁₁ independently represent a hydrogen atom, cyano, linear or branched C₁-C₃ alkyl and C₁-C₄ haloalkyl;
R₉ is chosen from a hydrogen atom, halogen atoms, cyano, linear or branched C₁-C₄ alkyl, C₁-C₄ haloalkyl, C₃-C₇ cycloalkyl, —C₁-C₃ alkyl-C₃-C₇ cycloalkyl, 5- to 6-membered monocyclic heteroaryl containing 1, 2 or 3 nitrogen atoms, and 5- to 7-membered heterocyclyl containing 1, 2 or 3 nitrogen atoms, the alkyl, cycloalkyl, heteroaryl and heterocyclyl being independently unsubstituted or substituted by one or more substituents selected from cyano or —OR₁₀, wherein R₁₀ represents linear or branched C₁-C₃ alkyl, or
R₉ is chosen from —S(O)₂R₁₃, —C(O)OR₁₃ and —NR₁₃R₁₄
wherein R₁₃ and R₁₄ are each independently a hydrogen atom or linear or branched C₁-C₄ alkyl, or when two —CR₉ adjacent groups are present, both —CR₉ and the carbon atoms to which they are bonded optionally form a 5- to 9-membered aryl group, the aryl being unsubstituted or substituted by one or more substituents selected from halogen atoms or linear or branched C₁-C₄ alkyl,
provided that when m is zero, R₈ is other than an —OR₁₃.

21. The method according to claim 20, wherein:
m is 0 or 1;
Z is NR₅;
X is a nitrogen atom and Y is —CR₉, or Y is a nitrogen atom and X is —CR₉;
R₁ is a hydrogen atom;
R₂ is a hydrogen atom;
R₃ is chosen from a hydrogen atom, halogen atoms, cyano, C₁-C₄ haloalkyl, cyclopropyl, phenyl unsubstituted or substituted by one or more halogen atoms, and 5- to 6-membered monocyclic heteroaryl containing 1, 2 or 3 heteroatoms selected from N and O, or
R₃ is chosen from —C(O)OR₁₃, —C(O)—NR₁₃R₁₄ and —NR₁₃C(O)R₁₄ wherein R₁₃ and R₁₄ each independently represent a hydrogen atom or methyl;
R₄ is a hydrogen atom;
R₅ is chosen from a hydrogen atom, methyl, ethyl and —C₁-C₃ alkyl-C₃-C₄ cycloalkyl;
R₆ and R₇ are each independently a hydrogen atom or methyl;
R₈ is chosen from linear or branched C₁-C₆ alkyl, C₁-C₄ haloalkyl, C₁-C₄ alkoxy, C₃-C₇ cycloalkyl, adamantyl, phenyl, 5- to 6-membered monocyclic heteroaryl containing 1, 2 or 3 heteroatoms selected from N, O and S, 5- to 7-membered heterocyclyl containing 1, 2 or 3 heteroatoms selected from N, O and S, and bicyclyl containing a phenyl bonded directly to a 5- to 7-membered heterocyclyl containing at least one heteroatom selected from O, S and N, the alkyl, haloalkyl, alkoxy, cycloalkyl, adamantyl, phenyl, heteroaryl, heterocyclyl and bicyclyl being independently unsubstituted or substituted by one or more substituents selected from halogen atoms, hydroxyl, linear or branched C₁-C₆ alkyl, phenyl, pyridyl, —S(O)₂R₁₀, —C(O)OR₁₀, —C(O)—(CH₂)ₙ—R₁₀, —NR₁₀R₁₁—C(O)—(CH₂)ₙ—NR₁₀R₁₁, —(C₁-C₄ alkyl)-CN, and —(C₁-C₄ alkyl)-C(O)NR′R″ wherein R′ and R″ are the same or different and are selected from hydrogen atoms and linear or branched C₁-C₄ alkyl; wherein n is 0 or 1, and
R₁₀ and R₁₁, are each independently chosen from a hydrogen atom, cyano, linear or branched C₁-C₆ alkyl, C₁-C₄ haloalkyl, C₃-C₇ cycloalkyl, phenyl, 5- to 6-membered monocyclic heteroaryl containing 1, 2 or 3 heteroatoms selected from N, O and S, 5- to 6-membered, heterocyclyl containing 1, 2 or 3 nitrogen atoms, and bicyclyl containing a monocyclic C₅-C₆ aryl or heteroaryl bonded directly to a 5- to 6-membered cycloalkyl or heterocyclyl, said heteroaryl or heterocyclyl containing 1, 2 or 3 nitrogen atoms, the alkyl, haloalkyl, cycloalkyl, phenyl, heteroaryl, heterocyclyl and bicyclyl being unsubstituted or substituted by one or more substituents selected from halogen atoms, hydroxyl, cyano, linear or branched C₁-C₆ alkyl, C₁-C₄ haloalkyl, phenyl unsubstituted or substituted by one or more halogen atoms, 5- to 6-membered monocyclic heteroaryl containing 1, 2 or 3 nitrogen atoms said heteroaryl being unsubstituted or substituted by one or more linear or branched C₁-C₃ alkyl, and C₃-C₇ heterocycloalkyl ketone containing 1, 2 or 3 nitrogen atoms, said heterocycloalkyl ketone being unsubstituted or substituted by one or more linear or branched C₁-C₃ alkyl, or
R₈ is —OR₁₃ wherein R₁₃ is C₁-C₃ alkyl unsubstituted or substituted by one or more phenyl, or
R₈ together with the R₅ and the nitrogen atom to which R₅ is bonded form a 5- to 7-membered, saturated heterocyclyl, which contains, as heteroatoms, one or two nitrogen atoms and which is substituted by —C(O)—(CH₂)ₙ—R₁₀ or —C(O)—(CH₂)ₙ—NR₁₀R₁₁, wherein n is 0 or 1, and R₁₀ and R₁₁ are independently chosen from a hydrogen atom, cyano, linear or branched C₁-C₃ alkyl and C₁-C₄ haloalkyl;
R₉ is chosen from a hydrogen atom, halogen atoms, cyano, linear or branched C₁-C₄ alkyl, C₃-C₇ cycloalkyl, 5- to 6-membered monocyclic heteroaryl containing 1, 2 or 3 nitrogen atoms, 5- to 7-membered heterocyclyl containing 1, 2 or 3 nitrogen atoms, the alkyl, cycloalkyl, heteroaryl, and heterocyclyl being unsubstituted or substituted by one or more substituents selected from cyano or —OR₁₀ wherein R₁₀ is linear or branched C₁-C₃ alkyl, or
R₉ is chosen from —S(O)₂R₁₃, —C(O)OR₁₃ and —NR₁₃R₁₄,
wherein R₁₃ and R₁₄ are each independently chosen from a hydrogen atom and linear or branched C₁-C₄ alkyl, or when two —CR₉ adjacent groups are present, both —CR₉ and the carbon atoms to which they are bonded optionally form a 5- to 6-membered aryl group, the aryl being unsubstituted or substituted by one or more substituents selected from halogen atoms and linear or branched C₁-C₄ alkyl,
provided that when m is zero, R₈ is other than an —OR₁₃.

22. The method according to claim 1, comprising administering to a subject in need thereof an effective amount of a compound of formula (I″), 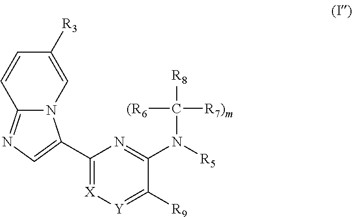
wherein X is a nitrogen atom and Y is —CR₉, or Y is a nitrogen atom and X is CR₉;
R₃ is a hydrogen atom, halogen atoms, cyano, —C(O)OR′, C₃-C₄ cycloalkyl, pyridinyl, pyrazolyl, or phenyl, which phenyl is unsubstituted or substituted with halogen atoms, wherein R′ is a hydrogen atom or linear or branched C₁-C₄ alkyl;
R₅ is a hydrogen atom, methyl, ethyl, —(CH₂)-cyclopropyl; or R₅ together with R₈ and the nitrogen atom to which R₅ is bonded form:
a 1,4-diazepane-1-yl group, which 1,4-diazepane-1-yl is unsubstituted or substituted by —C(O)CH₂CN, or
a pyrrolidinyl group, which pyrrolidinyl is unsubstituted or substituted by pyridinyl or —C(O)N(CH₂CH₃)₂;
R₆ and R₇ are each independently a hydrogen atom or linear or branched C₁-C₃ alkyl unsubstituted or substituted by C₁-C₂ alkoxy;
R₈ together with R₅ and the nitrogen atom to which R₅ is bonded form said 1,4-diazepane-1-yl group or pyrrolidinyl group; or R₈ is a hydrogen atom, linear or branched C₁-C₅ alkyl, —(C₁-C₅ alkyl)-(C₁-C₂ alkoxy), cyclohexyl, adamantyl, phenyl, pyridinyl, 5,6,7,8-tetrahydroquinolinyl, tetrahydropyranyl or chromanyl, which cyclohexyl, adamantyl, phenyl, pyridinyl, tetrahydroquinolinyl, tetrahydropyranyl and chromanyl are independently unsubstituted or substituted by one ore more substituents selected from halogen atoms, hydroxyl, —CH₂CN, linear or branched C₁-C₃ alkyl and C₁-C₄ haloalkyl; or R₈ is piperidinyl, piperazinyl, pyrrolidinyl or pyrrolidin-2-onyl, which piperidinyl, piperazinyl, pyrrolidinyl and pyrrolidin-2-onyl are independently unsubstituted or substituted by one or more substituents selected from linear or branched C₁-C₃ alkyl, phenyl, pyridinyl, triazololyl, thiazolyl, —S(O)₂₋(CH₂)ₙR₁₀, —C(O)—(CH₂)ₙOR₁₀, —C(O)—(CH₂)ₙR₁₀, —C(O)—(CH₂)ₙ—N(R₁₀)—(CH₂)ₙR₁₁, —(C₁-C₄ alkyl)-CN, and —(C₁-C₄ alkyl)-C(O)NR₁₀R₁₁, wherein n is independently 0 or 1, and wherein the phenyl, pyridinyl, triazololyl and thiazolyl are independently unsubstituted or substituted by one or more substituents selected from halogen atoms, hydroxyl, cyano, linear or branched C₁-C₃ alkyl and C₁-C₄ haloalkyl, and wherein
R₁₁ is a hydrogen atom or linear or branched C₁-C₄ alkyl, and
R₁₀ is chosen from a hydrogen atom; cyano; linear or branched C₁-C₅ alkyl; C₁-C₄ haloalkyl; C₁-C₅ hydroxyalkyl; —(C₁-C₄ alkyl)(C₁-C₂ alkoxy); C₃-C₆ cycloalkyl, which cycloalkyl is unsubstituted or substituted by one or more substituents selected from halogen atoms, hydroxyl, cyano, linear or branched C₁-C₃ alkyl, C₁-C₃ haloalkyl, —(C₁-C₄ alkyl)-CN and phenyl; pyridinyl, which pyridinyl is unsubstituted or substituted by one or more substituents selected from halogen atoms or cyano; phenyl, which phenyl is unsubstituted or substituted by one or more substituents selected from halogen atoms, cyano, linear or branched C₁-C₃ alkyl and C₁-C₄ hydroxyalkyl; pyrrolidinyl, which pyrrolidinyl is unsubstituted or substituted by one or more substituents selected from halogen atoms or cyano; pyrimidinyl, which pyrimidinyl is unsubstituted or substituted by one or more substituents selected from halogen atoms, hydroxyl and linear or branched C₁-C₃ alkyl; thiazolyl, which thiazolyl is unsubstituted or substituted by one or more substituents selected from halogen atoms, cyano and linear or branched C₁-C₃ alkyl; an imidazolyl, —(C₁-C₅ alkyl)-(phenyl), wherein the phenyl of the —(C₁-C₅ alkyl)-(phenyl) is unsubstituted or substituted by one or more substituents selected from halogen atoms or C₁-C₂ alkoxy; —(C₁-C₅ hydroxyalkyl)-(phenyl), wherein the phenyl of the —(C₁-C₅ hydroxyalkyl)-(phenyl) is unsubstituted or substituted by one or more substituents selected from halogen atoms or C₁-C₂ alkoxy; 1-thia-2,3-diazolyl, which 1-thia-2,3-diazolyl is unsubstituted or substituted by one or more linear or branched C₁-C₃ alkyl s; benzimidazolyl, which benzimidazolyl is unsubstituted or substituted by one or more linear or branched C₁-C₃ alkyl s; chromanyl; 2,2-dimethyl-1,3-dioxolanyl: imidazolyl; an indolyl; tetrahydropyranyl; tetrazolyl; triazolyl; pyrazolyl, benzoyl and pyrimidine-2,4(1H,3H)-dionyl.
R₉ is a hydrogen atom; halogen atoms; cyano; C₁-C₄ haloalkyl; linear or branched C₁-C₃ alkyl; C₃-C₄ cycloalkyl; pyridinyl, which pyridinyl is unsubstituted or substituted by one or more substituents selected from hydroxyl and C₁-C₃ alkoxy; triazolyl; tetrazolyl, which tetrazolyl is unsubstituted or substituted by one or more linear or branched C₁-C₃ alkyl s; pyrrolidinyl, which pyrrolidinyl is unsubstituted or substituted by one or more —C(O)OR′₁₃; piperazinyl, which piperazinyl is unsubstituted or substituted by one or more substituents selected from linear or branched C₁-C₃ alkyl and —S(O)₂R″₁₃; morpholinyl; —S(O)₂R₁₃; —OR₁₃; —C(O)OR₁₃; —NR₁₃R₁₄ and —C(O)—(CH₂)ₙ—NR₁₃R₁₄; or when Y is a R₉ group, the two adjacent R₉, together with the carbon atoms to which they are attached, may form a benzene ring; wherein n is independently 0 or 1 and each R₁₃ and R₁₄ are independently chosen from a hydrogen atom, linear or branched C₁-C₃ alkyl, —(C₁-C₃ alkyl)-(C₁-C₂ alkoxy), —(C₁-C₃ alkyl)-(pirrolidinyl); and —(C₁-C₃ alkyl)-(morpholinyl); R′₁₃ is a hydrogen atom or linear or branched C₁-C₃ alkyl; and R″₁₃ is a linear or branched C₁-C₃ alkyl.

23. The method according to claim 1, comprising administering to a subject in need thereof an effective amount of a compound of formula (I′), 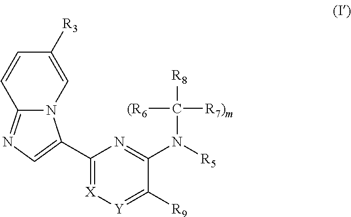
wherein X is a nitrogen atom and Y is —CR₉, or Y is a nitrogen atom and X is CR₉;
R₃ is a hydrogen atom, halogen atoms, cyano, —C(O)OR′, C₃-C₄ cycloalkyl, pyridinyl, pyrazolyl, or phenyl, which phenyl is unsubstituted or substituted with halogen atoms, wherein R′ is a hydrogen atom or linear or branched C₁-C₄ alkyl;
R₅ is a hydrogen atom, methyl, ethyl, —CH₂-cyclopropyl, or R₅ together with R₈ and the nitrogen atom to which R₅ is bonded form a 1,4-diazepane-1-yl group, which 1,4-dizepane-1-yl is unsubstituted or substituted with —C(O)CH₂CN;
R₆ and R₇ are each independently a hydrogen atom or methyl;
R₉ is a hydrogen atom, halogen atoms, or methyl, ethyl, piperazinyl, pyridonyl or pyridinyl, which pyridinyl is unsubstituted or substituted with C₁-C₂ alkoxy, or when Y is a R₉ group, the two adjacent R₉, together with the carbon atoms to which they are attached, may form a benzene ring;
R₈ together with R₅ and the nitrogen atom to which R₅ is bonded form a said 1,4-diazepane-1-yl group, or R₈ is a hydrogen atom, linear or branched C₁-C₅ alkyl, cyclohexyl, adamantyl, pyranyl, piperidinyl, chromanyl, —(C₁-C₅ alkyl)-phenyl, —(C₁-C₅ alkyl)-cyclohexyl, —(C₁-C₅ alkyl)-(C₁-C₂ alkoxy), -A-SO₂—R′, -A-A′, -A-L-C(O)NR′R″, -A-L-CN, -A-C(O)—N(C₁-C₂ alkyl)-L-CN, -A-C(O)—NR′R″, -A-C(O)-A″, -A-C(O)—R″′, -A-CO₂—R′, -A-C(O)-L-A″′, -A-C(O)-L-CN, and -A-C(O)-L-O—R′, wherein R′ and R″ are the same or different and each is independently a hydrogen atom or linear or branched C₁-C₄ alkyl, and R″′ is chosen from linear or branched C₁-C₅ alkyl, C₁-C₂ haloalkyl and C₁-C₄ hydroxyalkyl, the cyclohexyl, adamantyl, pyranyl, piperidinyl and chromanyl being independently unsubstituted or substituted with halogen atoms, hydroxyl, linear or branched C₁-C₂ alkyl, or C₁-C₂ alkoxy, and wherein
L is linear or branched C₁-C₅ alkylene,
A is piperidinyl or pyrrolidinyl, which are independently unsubstituted or substituted with C₁-C₂ alkyl,
A′ is phenyl or pyridinyl, which are independently unsubstituted or substituted with 1 or 2 halogen atoms or CN,
A″ is pyrrolidinyl, pyridinyl, pyrazolyl or cyclopropyl, the pyrrolidinyl, pyridinyl, pyrazolyl and cyclopropyl being unsubstituted or substituted with 1 or 2 halogen atoms or cyano, and
A″′ is an imidazolyl.

24. The method according to claim 1, wherein the compound of formula (I) is chosen from:
3-(4-{[(1S)-1-phenylethyl]amino}pyrimidin-2-yl)imidazo[1,2-a]pyridine-6-carbonitrile;
3-(4-{[(1R)-1-phenylethyl]amino}pyrimidin-2-yl)imidazo[1,2-a]pyridine-6-carbonitrile;
3-[4-(Benzylamino)pyrimidin-2-yl]imidazo[1,2-a]pyridine-6-carbonitrile;
3-(4-{[(1S)-2-methoxy-1-methylethyl]amino}pyrimidin-2-yl)Imidazo[1,2-a]pyridine-6-carbonitrile;
3-{4-[(Cyclohexylmethyl)amino]pyrimidin-2-yl}imidazo[1,2-a]pyridine-6-carbonitrile;
3-{4-[(2-Methoxyethyl)amino]pyrimidin-2-yl}imidazo[1,2-a]pyridine-6-carbonitrile;
3-{4-[(1-Adamantylmethyl)amino]pyrimidin-2-yl}imidazo[1,2-a]pyridine-6-carbonitrile;
3-{4-[(2,2-Dimethylpropyl)amino]pyrimidin-2-yl}imidazo[1,2-a]pyridine-6-carbonitrile;
3-{5-Bromo-4-[(2,2-dimethylpropyl)amino]pyrimidin-2-yl}imidazo[1,2-a]pyridine-6-carbonitrile;
3-{4-[(2,2-Dimethylpropyl)amino]-5-piperazin-1-ylpyrimidin-2-yl}imidazo[1,2-a]pyridine-6-carbonitrile;
3-[4-[(2,2-Dimethylpropyl)amino]-5-(2-methoxypyridin-4-yl)pyrimidin-2-yl]imidazo[1,2-a]pyridine-6-carbonitrile;
3-[4-[(2,2-Dimethylpropyl)amino]-5-(2-oxo-1,2-dihydropyridin-4-yl)pyrimidin-2-yl]imidazo[1,2-a]pyridine-6-carbonitrile;
3-{4-[(2,2-Dimethylpropyl)amino]-5-pyridin-3-ylpyrimidin-2-yl}imidazo[1,2-a]pyridine-6-carbonitrile;
3-{4-[(3-Fluorobenzyl)amino]pyrimidin-2-yl}imidazo[1,2-a]pyridine-6-carbonitrile;
3-{4-[(4-Fluorobenzyl)amino]pyrimidin-2-yl}imidazo[1,2-a]pyridine-6-carbonitrile;
3-{4-[(2-Methylbenzyl)amino]pyrimidin-2-yl}imidazo[1,2-a]pyridine-6-carbonitrile,
Tert-butyl-2-({[2-(6-cyanoimidazo[1,2-a]pyridin-3-yl)pyrimidin-4-yl]amino}methyl)piperidine-1-carboxylate;
3-(4-{[(1-Acetylpiperidin-2-yl)methyl]amino}pyrimidin-2-yl)imidazo[1,2-a]pyridine-6-carbonitrile;
3-[4-(Tetrahydro-2H-pyran-4-ylamino)pyrimidin-2-yl]imidazo[1,2-a]pyridine-6-carbonitrile;
3-(4-(8-Fluorochroman-4-ylamino)pyrimidin-2-yl)imidazo[1,2-a]pyridine-6-carbonitrile;
3-[4-(Cyclohexylamino)pyrimidin-2-yl]imidazo[1,2-a]pyridine-6-carbonitrile;
3-{4-[(Trans-4-hydroxycyclohexyl)amino]pyrimidin-2-yl}imidazo[1,2-a]pyridine-6-carbonitrile;
3-{4-[(5-Hydroxy-2-adamantyl)amino]pyrimidin-2-yl}imidazo[1,2-a]pyridine-6-carbonitrile;
3-{4-[(5-Hydroxy-2-adamantyl)amino]pyrimidin-2-yl}imidazo[1,2-a]pyridine-6-carbonitrile;
2-{4-[(2,2-Dimethylpropyl)amino]quinazolin-2-yl}imidazo[1,2-a]pyridine-6-carbonitrile;
3-{4-[Benzyl(methyl)amino]pyrimidin-2-yl}imidazo[1,2-a]pyridine-6-carbonitrile;
3-(4-{[(1S)-1-phenylethyl]amino}pyrimidin-2-yl)imidazo[1,2-a]pyridine-6-carboxylic acid;
2-(6-Fluoroimidazo[1,2-a]pyridin-3-yl)-N-[(1S)-1-phenylethyl]pyrimidin-4-amine;
Trans-4-{[2-(6-fluoroimidazo[1,2-a]pyridin-3-yl)pyrimidin-4-yl]amino}cyclohexanol;
3-(4-{[(2S,7S)-5-hydroxy-2-adamantyl]amino}pyrimidin-2-yl)imidazo[1,2-a]pyridine-6-carbonitrile;
N-(2,2-dimethylpropyl)-2-imidazo[1,2-a]pyridin-3-ylpyrimidin-4-amine;
N-(2,2-dimethylpropyl)-2-imidazo[1,2-a]pyridin-3-yl-6-pyridin-3-ylpyrimidin-4-amine;
3-(6-{[(1S)-1-phenylethyl]amino}pyrazin-2-yl)imidazo[1,2-a]pyridine-6-carbonitrile;
3-{6-[(Cyclohexylmethyl)amino]pyrazin-2-yl}imidazo[1,2-a]pyridine-6-carbonitrile;
6-(6-Fluoroimidazo[1,2-a]pyridin-3-yl)-N-[(1S)-1-phenylethyl]pyrazin-2-amine;
6-(6-Chloroimidazo[1,2-a]pyridin-3-yl)-N-[(1S)-1-phenylethyl]pyrazin-2-amine;
(S)-6-(imidazo[1,2-a]pyridin-3-yl)-N-(1-phenylethyl)pyrazin-2-amine;
6-(6-Cyclopropylimidazo[1,2-a]pyridin-3-yl)-N-[(1S)-1-phenylethyl]pyrazin-2-amine;
N-[(1S)-1-phenylethyl]-6-(6-pyridin-3-ylimidazo[1,2-a]pyridin-3-yl)pyrazin-2-amine;
N-[(1S)-1-phenylethyl]-6-(6-pyridin-4-ylimidazo[1,2-a]pyridin-3-yl)pyrazin-2-amine;
N-[(1S)-1-phenylethyl]-6-[6-(1H-pyrazol-4-yl)imidazo[1,2-a]pyridin-3-yl]pyrazin-2-amine;
N-[(1S)-1-phenylethyl]-6-(6-phenylimidazo[1,2-a]pyridin-3-yl)pyrazin-2-amine;
6-[6-(4-Fluorophenyl)imidazo[1,2-a]pyridin-3-yl]-N-[(1S)-1-phenylethyl]pyRazin-2-amine;
3-(4-{[(3R)-1-(ethylsulfonyl)piperidin-3-yl]amino}pyrimidin-2-yl)imidazo[1,2-a]pyridine-6-carbonitrile;
3-(4-{[(3R)-1-(isopropylsulfonyl)piperidin-3-yl]amino}pyrimidin-2-yl)imidazo[1,2-a]pyridine-6-carbonitrile;
3-(4-{[(3R)-1-(cyanocetyl)piperidin-3-yl]amino}pyrimidin-2-yl)imidazo[1,2-a]pyridine-6-carbonitrile;
3-(4-{[(3R)-1-propionylpiperidin-3-yl]amino}pyrimidin-2-yl)imidazo[1,2-a]pyridine-6-carbonitrile;
3-[4-({(3R)-1-[(1-cyanocyclopropyl)carbonyl]piperidin-3-yl}amino)pyrimidin-2-yl]imidazo[1,2-a]pyridine-6-carbonitrile;
3-(4-{[(3R)-1-(methoxycetyl)piperidin-3-yl]amino}pyrimidin-2-yl)imidazo[1,2-a]pyridine-6-carbonitrile;
3-(4-{[(3R)-1-(3-hydroxy-3-methylbutanoyl)piperidin-3-yl]amino}pyrimidin-2-yl)imidazo[1,2-a]pyridine-6-carbonitrile,
3-(4-{[(3R)-1-(3,3-dimethylbutanoyl)piperidin-3-yl]amino}pyrimidin-2-yl)imidazo[1,2-a]pyridine-6-carbonitrile;
3-(4-{[(3R)-1-(1H-imidazol-4-ylcetyl)piperidin-3-yl]amino}pyrimidin-2-yl)imidazo[1,2-a]pyridine-6-carbonitrile;
3-[4-({(3R)-1-[(5-cyanopyridin-2-yl)carbonyl]piperidin-3-yl}amino)pyrimidin-2-yl]imidazo[1,2-a]pyridine-6-carbonitrile;
3-(4-{[(3R)-1-(3,3,3-trifluoropropanoyl)piperidin-3-yl]amino}pyrimidin-2-yl)imidazo[1,2-a]pyridine-6-carbonitrile;
(3R)-3-{[2-(6-cyanoimidazo[1,2-a]pyridin-3-yl)pyrimidin-4-yl]amino}-N,N-dimethyl piperidine-1-carboxamide;
3-{4-[((3R)-1-{[(2S,4S)-2-cyano-4-fluoropyrrolidin-1-yl]carbonyl}piperidin-3-yl)amino]pyrimidin-2-yl}imidazo[1,2-a]pyridine-6-carbonitrile;
3-(4-{[(3R)-1-(5-cyanopyridin-2-yl)piperidin-3-yl]amino}pyrimidin-2-yl)imidazo[1,2-a]pyridine-6-carbonitrile;
3-(4-{[(3R)-1-(4-cyano-2-fluorophenyl)piperidin-3-yl]amino}pyrimidin-2-yl)imidazo[1,2-a]pyridine-6-carbonitrile,
3-{4-[[(3R)-1-(cyanoacetyl)piperidin-3-yl](methyl)amino]pyrimidin-2-yl}imidazo[1,2-a]pyridine-6-carbonitrile;
3-(4-{[(3S)-1-(cyanoacetyl)piperidin-3-yl]amino}pyrimidin-2-yl)imidazo[1,2-a]pyridine-6-carbonitrile;
Cis-3-(4-{1-(cyanoacetyl)-4-methylpiperidin-3-yl]amino}pyrimidin-2-yl)imidazo[1,2-a]pyridine-6-carbonitrile,
Cis-3-(4-{1-(ethylsulfonyl)-4-methylpiperidin-3-yl]amino}pyrimidin-2-yl)imidazo[1,2-a]pyridine-6-carbonitrile;
3-{4-[[1-(Ethylsulfonyl)-4-methylpiperidin-3-yl](methyl)amino]pyrimidin-2-yl}imidazo[1,2-a]pyridine-6-carbonitrile;
3-((3R)-3-{[2-(6-fluoroimidazo[1,2-a]pyridin-3-yl)pyrimidin-4-yl]amino}piperidin-1-yl)-3-oxopropanenitrile,
(R)-1-(3-(2-(6-fluoroimidazo[1,2-a]pyridin-3-yl)pyrimidin-4-ylamino)piperidine-1-carbonyl)cyclopropanecarbonitrile;
2-(6-Fluoroimidazo[1,2-a]pyridin-3-yl)-N-[(3R)-1-(3,3,3-trifluoropropanoyl)piperidin-3-yl]pyrimidin-4-amine;
2-(6-Fluoroimidazo[1,2-a]pyridin-3-yl)-N-[(3R)-1-(1H-pyrazol-4ylcarbonyl)piperidin-3-yl]pyrimidin-4-amine;
(3R)—N-(cyanomethyl)-3-{[2-(6-fluoroimidazo[1,2-a]pyridin-3-yl)pyrimidin-4-yl]amino}-N-methylpiperidine-1-carboxamide;
2-((3R)-3-{[2-(6-fluoroimidazo[1,2-a]pyridin-3-yl)pyrimidin-4-yl]amino}piperidin-1-yl)-2-methylpropanmide;
2-(6-Fluoroimidazo[1,2-a]pyridin-3-yl)-N-methyl-N-[(3R)-1-(3,3,3-trifluoro propanoyl)piperidin-3-yl]pyrimidin-4-amine;
(R)-3-(3-((2-(6-fluoroimidazo[1,2-a]pyridin-3-yl)pyrimidin-4-yl)(methyl)amino)piperidin-1-yl)-3-oxopropanenitrile;
(R)-1-(3-((2-(6-fluoroimidazo[1,2-a]pyridin-3-yl)pyrimidin-4-yl)(methyl)amino)piperidine-1-carbonyl)cyclopropanecarbonitrile;
3-((3R)-3-{ethyl[2-(6-fluoroimidazo[1,2-a]pyridin-3-yl)pyrimidin-4-yl]amino}piperidin-1-yl)-3-oxopropanenitrile;
N-ethyl-2-(6-fluoroimidazo[1,2-a]pyridin-3-yl)-N-[(3R)-1-(3,3,3-trifluoropropanoyl)piperidin-3-yl]pyrimidin-4-amine;
3-((3R)-3-{(cyclopropylmethyl)[2-(6-fluoroimidazo[1,2-a]pyridin-3-yl)pyrimidin-4-yl]amino}piperidin-1-yl)-3-oxopropanenitrile;
N-(cyclopropylmethyl)-2-(6-fluoroimidazo[1,2-a]pyridin-3-yl)-N-[(3R)-1-(3,3,3-trifluoropropanoyl)piperidin-3-yl]pyrimidin-4-amine;
3-((3R)-3-{[5-fluoro-2-(6-fluoroimidazo[1,2-a]pyridin-3-yl)pyrimidin-4-yl]amino}piperidin-1-yl)-3-oxopropanenitrile;
5-Fluoro-2-(6-fluoroimidazo[1,2-a]pyridin-3-yl)-N-[(3R)-1-(3,3,3-trifluoropropanoyl)piperidin-3-yl]pyrimidin-4-amine;
3-((3R)-3-{[2-(6-fluoroimidazo[1,2-a]pyridin-3-yl)-5-methylpyrimidin-4-yl]amino}piperidin-1-yl)-3-oxopropanenitrile;
2-(6-fluoroimidazo[1,2-a]pyridin-3-yl)-5-methyl-N-[(3R)-1-(3,3,3-trifluoropropanoyl)piperidin-3-yl]pyrimidin-4-amine;
3-((3R)-3-{[2-(6-fluoroimidazo[1,2-a]pyridin-3-yl)pyrimidin-4-yl]amino}pyrrolidin-1-yl)-3-oxopropanenitrile;
3-{4-[4-(cyanocetyl)-1,4-diazepan-1-yl]pyrimidin-2-yl}imidazo[1,2-a]pyridine-6-carbonitrile;
3-((3R)-3-{[5-chloro-2-(6-fluoroimidazo[1,2-a]pyridin-3-yl)pyrimidin-4-yl]amino}piperidin-1-yl)-3-oxopropanenitrile;
3-((3R)-3-{[2-(6-fluoroimidazo[1,2-a]pyridin-3-yl)pyrimidin-4-yl]amino}piperidin-1-yl)-2,2-dimethylpropanenitrile;
2-((3R)-3-{[2-(6-fluoroimidazo[1,2-a]pyridin-3-yl)pyrimidin-4-yl]amino}piperidin-1-yl)cetmide,
(S)-2-(6-fluoroimidazo[1,2-a]pyridin-3-yl)-N-(1-(5-fluoropyridin-2-yl)ethyl)pyrimidin-4-amine;
2-(6-fluoroimidazo[1,2-a]pyridin-3-yl)-N-(pyridin-2-ylmethyl)pyrimidin-4-amine;
(S)-2-(6-fluoroimidazo[1,2-a]pyridin-3-yl)-N-(1-(4-fluorophenyl)butyl)pyrimidin-4-amine;
(R)-3-(2-(6-fluoroimidazo[1,2-a]pyridin-3-yl)pyrimidin-4-ylamino)-1-propylpyrrolidin-2-one;
6-fluoro-3-(4-(2-(pyridin-2-yl)pyrrolidin-1-yl)pyrimidin-2-yl)imidazo[1,2-a]pyridine;
(S)—N,N-diethyl-1-(2-(6-fluoroimidazo[1,2-a]pyridin-3-yl)pyrimidin-4-yl)pyrrolidine-2-carboxamide;
(R)—N,N-diethyl-1-(2-(6-fluoroimidazo[1,2-a]pyridin-3-yl)pyrimidin-4-yl)pyrrolidine-2-carboxamide;
2-((1r,4r)-4-(2-(6-fluoroimidazo[1,2-a]pyridin-3-yl)pyrimidin-4-ylamino)cyclohexyl)acetonitrile;
(R)-2-(3-(2-(6-fluoroimidazo[1,2-a]pyridin-3-yl)pyrimidin-4-ylamino)piperidin-1-ylsulfonyl)acetonitrile,
(R)-4,4,4-trifluoro-1-(3-(2-(6-fluoroimidazo[1,2-a]pyridin-3-yl)pyrimidin-4-ylamino)piperidin-1-yl)butan-1-one;
((R)-3-(2-(6-fluoroimidazo[1,2-a]pyridin-3-yl)pyrimidin-4-ylamino)piperidin-1-yl)((1R,2R)-2-phenylcyclopropyl)methanone;
(R)-1-((R)-3-(2-(6-fluoroimidazo[1,2-a]pyridin-3-yl)pyrimidin-4-ylamino)piperidin-1-yl)-2-hydroxy-3,3-dimethylbutan-1-one;
(R)-2-cyclopentyl-1-(3-(2-(6-fluoroimidazo[1,2-a]pyridin-3-yl)pyrimidin-4-ylamino)piperidin-1-yl)ethanone;
(R)-1-(2-(3-(2-(6-fluoroimidazo[1,2-a]pyridin-3-yl)pyrimidin-4-ylamino)piperidin-1-yl)-2-oxoethyl)-5-methylpyrimidine-2,4(1H,3H)-dione;
(R)-1-(3-(2-(6-fluoroimidazo[1,2-a]pyridin-3-yl)pyrimidin-4-ylamino)piperidin-1-yl)-2-(1H-1,2,4-trizol-1-yl)ethanone;
(R)-(3-(2-(6-fluoroimidazo[1,2-a]pyridin-3-yl)pyrimidin-4-ylamino)piperidin-1-yl)(1-methylcyclohexyl)methanone;
(2,2-difluorocyclopropyl)((R)-3-(2-(6-fluoroimidazo[1,2-a]pyridin-3-yl)pyrimidin-4-ylamino)piperidin-1-yl)methanone;
((R)-3-(2-(6-fluoroimidazo[1,2-a]pyridin-3-yl)pyrimidin-4-ylamino)piperidin-1-yl)((1R,2S)-2-hydroxycyclopentyl)methanone;
(R)-1-(3-(2-(6-fluoroimidazo[1,2-a]pyridin-3-yl)pyrimidin-4-ylamino)piperidin-1-yl)-2-(1H-pyRazol-1-yl)ethanone;
(R)-cyclohexyl(3-(2-(6-fluoroimidazo[1,2-a]pyridin-3-yl)pyrimidin-4-ylamino)piperidin-1-yl)methanone;
(R)-(3-(2-(6-fluoroimidazo[1,2-a]pyridin-3-yl)pyrimidin-4-ylamino)piperidin-1-yl)(4-methyl-1,2,3-thiadiazol-5-yl)methanone;
(R)-1-(3-(2-(6-fluoroimidazo[1,2-a]pyridin-3-yl)pyrimidin-4-ylamino)piperidin-1-yl)-2-(4-(hydroxymethyl)phenyl)ethanone;
(S)-1-((R)-3-(2-(6-fluoroimidazo[1,2-a]pyridin-3-yl)pyrimidin-4-ylamino)piperidin-1-yl)-3-phenylbutan-1-one;
(R)-1-((R)-3-(2-(6-fluoroimidazo[1,2-a]pyridin-3-yl)pyrimidin-4-ylamino)piperidin-1-yl)-3-phenylbutan-1-one;
(R)-1-(3-(2-(6-fluoroimidazo[1,2-a]pyridin-3-yl)pyrimidin-4-ylamino)piperidin-1-yl)-3-(4-methoxyphenyl)propan-1-one;
(R)-(5-fluoro-2,6-dihydroxypyrimidin-4-yl)(3-(2-(6-fluoroimidazo[1,2-a]pyridin-3-yl)pyrimidin-4-ylamino)piperidin-1-yl)methanone;
(R)-2-(3-chlorophenyl)-1-((R)-3-(2-(6-fluoroimidazo[1,2-a]pyridin-3-yl)pyrimidin-4-ylamino)piperidin-1-yl)-2-hydroxyethanone;
(R)-(3-(2-(6-fluoroimidazo[1,2-a]pyridin-3-yl)pyrimidin-4-ylamino)piperidin-1-yl)(pyrimidin-5-yl)methanone;
(R)-(3-(2-(6-fluoroimidazo[1,2-a]pyridin-3-yl)pyrimidin-4-ylamino)piperidin-1-yl)(1-(trifluoromethyl)cyclobutyl)methanone;
(R)-(3-(2-(6-fluoroimidazo[1,2-a]pyridin-3-yl)pyrimidin-4-ylamino)piperidin-1-yl)(1-hydroxycyclopropyl)methanone;
(R)-2-(benzyloxy)-1-(3-(2-(6-fluoroimidazo[1,2-a]pyridin-3-yl)pyrimidin-4-ylamino)piperidin-1-yl)ethanone;
(R)-(3-(2-(6-fluoroimidazo[1,2-a]pyridin-3-yl)pyrimidin-4-ylamino)piperidin-1-yl)(1H-indol-2-yl)methanone;
(R)-1-(3-(2-(6-fluoroimidazo[1,2-a]pyridin-3-yl)pyrimidin-4-ylamino)piperidin-1-yl)-5-(4-fluorophenyl)pentan-1-one;
(R)-2-(2-chlorophenyl)-1-((R)-3-(2-(6-fluoroimidazo[1,2-a]pyridin-3-yl)pyrimidin-4-ylamino)piperidin-1-yl)-2-hydroxyethanone;
(R)-1-(3-(2-(6-fluoroimidazo[1,2-a]pyridin-3-yl)pyrimidin-4-ylamino)piperidin-1-yl)-2-(methylamino)ethanone;
(R)-1-(3-(2-(6-fluoroimidazo[1,2-a]pyridin-3-yl)pyrimidin-4-ylamino)piperidin-1-yl)-2-(2-methoxyethoxy)ethanone;
(R)-1-(3-(2-(6-fluoroimidazo[1,2-a]pyridin-3-yl)pyrimidin-4-ylamino)piperidin-1-yl)-2-(2-methylthiazol-4-yl)ethanone;
(R)-(3-(2-(6-fluoroimidazo[1,2-a]pyridin-3-yl)pyrimidin-4-ylamino)piperidin-1-yl)(tetrahydro-2H-pyran-4-yl)methanone;
(R)-1-(3-(2-(6-fluoroimidazo[1,2-a]pyridin-3-yl)pyrimidin-4-ylamino)piperidin-1-yl)-2-(2-methyl-1H-benzo[d]imidazol-1-yl)ethanone;
chroman-3-yl((R)-3-(2-(6-fluoroimidazo[1,2-a]pyridin-3-yl)pyrimidin-4-ylamino)piperidin-1-yl)methanone;
(R)—N-(2-(3-(2-(6-fluoroimidazo[1,2-a]pyridin-3-yl)pyrimidin-4-ylamino)piperidin-1-yl)-2-oxoethyl)-N-methylbenzamide;
(R)-2-(3-chlorophenoxy)-1-(3-(2-(6-fluoroimidazo[1,2-a]pyridin-3-yl)pyrimidin-4-ylamino)piperidin-1-yl)ethanone;
(R)-1-(3-(2-(6-fluoroimidazo[1,2-a]pyridin-3-yl)pyrimidin-4-ylamino)piperidin-1-yl)-2,2-bis(hydroxymethyl)butan-1-one;
(R)-1-(3-(2-(6-fluoroimidazo[1,2-a]pyridin-3-yl)pyrimidin-4-ylamino)piperidin-1-yl)-2-(2H-tetrazol-5-yl)ethanone;
N-(1-(1H-1,2,4-triazol-3-yl)piperidin-3-yl)-2-(6-fluoroimidazo[1,2-a]pyridin-3-yl)pyrimidin-4-amine;
(R)-2-(3-(2-(6-fluoroimidazo[1,2-a]pyridin-3-yl)pyrimidin-4-ylamino)piperidin-1-yl)thiazole-5-carbonitrile;
(R)-1-((R)-3-(2-(6-fluoroimidazo[1,2-a]pyridin-3-yl)pyrimidin-4-ylamino)piperidin-1-yl)-2,3-dihydroxypropan-1-one;
(S)-1-((R)-3-(2-(6-fluoroimidazo[1,2-a]pyridin-3-yl)pyrimidin-4-ylamino)piperidin-1-yl)-2,3-dihydroxypropan-1-one;
(R)-3-(3-(2-(6-fluoroimidazo[1,2-a]pyridin-3-yl)-5-methylpyrimidin-4-ylamino)pyrrolidin-1-yl)-3-oxopropanenitrile;
(S)-2-(6-fluoroimidazo[1,2-a]pyridin-3-yl)-N-(1-(5-fluoropyridin-2-yl)ethyl)-5-methylpyrimidin-4-amine;
2-(6-fluoroimidazo[1,2-a]pyridin-3-yl)-N4-((5-fluoropyridin-2-yl)methyl)pyrimidine-4,5-diamine;
2-(6-fluoroimidazo[1,2-a]pyridin-3-yl)-N4-(5,6,7,8-tetRahydroquinolin-5-yl)pyrimidine-4,5-diamine;
(R)-2-(6-fluoroimidazo[1,2-a]pyridin-3-yl)-N4-(3-methylbutan-2-yl)pyrimidine-4,5-diamine;
2-(6-fluoroimidazo[1,2-a]pyridin-3-yl)-N4-(1-methoxypropan-2-yl)pyrimidine-4,5-diamine;
2-((1r,4r)-4-(5-amino-2-(6-fluoroimidazo[1,2-a]pyridin-3-yl)pyrimidin-4-ylamino)cyclohexyl)acetonitrile;
(1r,4r)-4-(5-amino-2-(6-fluoroimidazo[1,2-a]pyridin-3-yl)pyrimidin-4-ylamino)-1-methylcyclohexanol;
2-(6-fluoroimidazo[1,2-a]pyridin-3-yl)-N4-(1-(5-fluoropyridin-2-yl)-2-methoxyethyl)pyrimidine-4,5-diamine;
(S)-2-(6-fluoroimidazo[1,2-a]pyridin-3-yl)-4-(1-(5-fluoropyridin-2-yl)ethylamino)pyrimidine-5-carboxylic acid;
(S)-2-(6-fluoroimidazo[1,2-a]pyridin-3-yl)-4-(1-(5-fluoropyridin-2-yl)ethylamino)pyrimidine-5-carboxamide;
(S)-2-(6-fluoroimidazo[1,2-a]pyridin-3-yl)-4-((1-(5-fluoropyridin-2-yl)ethyl)(methyl)amino)pyrimidine-5-carboxylic acid;
2-(6-fluoroimidazo[1,2-a]pyridin-3-yl)-4-(pyridin-3-ylmethylamino)pyrimidine-5-carboxylic acid;
2-(6-fluoroimidazo[1,2-a]pyridin-3-yl)-4-(pyridin-3-ylmethylamino)pyrimidine-5-carboxamide;
2-(6-fluoroimidazo[1,2-a]pyridin-3-yl)-4-((5-fluoropyridin-2-yl)methylamino)pyrimidine-5-carboxylic acid;
2-(6-fluoroimidazo[1,2-a]pyridin-3-yl)-4-((5-fluoropyridin-2-yl)methylamino)pyrimidine-5-carboxamide;
(S)-2-(6-fluoroimidazo[1,2-a]pyridin-3-yl)-4-(1-(4-fluorophenyl)butylamino)pyrimidine-5-carboxylic acid;
(S)-2-(6-fluoroimidazo[1,2-a]pyridin-3-yl)-4-(1-(4-fluorophenyl)butylamino)pyrimidine-5-carboxamide;
4-((1r,4r)-4-(cyanomethyl)cyclohexylamino)-2-(6-fluoroimidazo[1,2-a]pyridin-3-yl)pyrimidine-5-carboxylic acid;
4-((1r,4r)-4-(cyanomethyl)cyclohexylamino)-2-(6-fluoroimidazo[1,2-a]pyridin-3-yl)pyrimidine-5-carboxamide;
((R)-2,2-dimethyl-1,3-dioxolan-4-yl)((R)-3-(2-(6-fluoroimidazo[1,2-a]pyridin-3-yl)-5-methylpyrimidin-4-ylamino)piperidin-1-yl)methanone;
(R)-1-((R)-3-(2-(6-fluoroimidazo[1,2-a]pyridin-3-yl)-5-methylpyrimidin-4-ylamino)piperidin-1-yl)-2,3-dihydroxypropan-1-one;
(R)-3-(3-(5-amino-2-(6-fluoroimidazo[1,2-a]pyridin-3-yl)pyrimidin-4-ylamino)piperidin-1-yl)-3-oxopropanenitrile;
(R)-3-(3-(5-cyclopropyl-2-(6-fluoroimidazo[1,2-a]pyridin-3-yl)pyrimidin-4-ylamino)piperidin-1-yl)-3-oxopropanenitrile;
(R)-4-(1-(2-cyanoacetyl)piperidin-3-ylamino)-2-(6-fluoroimidazo[1,2-a]pyridin-3-yl)pyrimidine-5-carbonitrile;
(R)-3-(3-(2-(6-fluoroimidazo[1,2-a]pyridin-3-yl)-5-(methylsulfonyl)pyrimidin-4-ylamino)piperidin-1-yl)-3-oxopropanenitrile;
(R)-ethyl 4-(1-(2-cyanoacetyl)piperidin-3-ylamino)-2-(6-fluoroimidazo[1,2-a]pyridin-3-yl)pyrimidine-5-carboxylate;
(R)-4-(1-(2-cyanoacetyl)piperidin-3-ylamino)-2-(6-fluoroimidazo[1,2-a]pyridin-3-yl)pyrimidine-5-carboxylic acid;
(R)-4-(1-(2-cyanoacetyl)piperidin-3-ylamino)-2-(6-fluoroimidazo[1,2-a]pyridin-3-yl)pyrimidine-5-carboxamide;
(R)-3-(3-(2-(6-fluoroimidazo[1,2-a]pyridin-3-yl)-5-methoxypyrimidin-4-ylamino)piperidin-1-yl)-3-oxopropanenitrile;
(R)-3-(3-(2-(6-fluoroimidazo[1,2-a]pyridin-3-yl)-6-(1H-1,2,4-triazol-1-yl)pyrimidin-4-ylamino)piperidin-1-yl)-3-oxopropanenitrile;
(R)-3-(3-(2-(6-fluoroimidazo[1,2-a]pyridin-3-yl)-6-(4-methylpiperazin-1-yl)pyrimidin-4-ylamino)piperidin-1-yl)-3-oxopropanenitrile;
(R)-3-(3-(2-(6-fluoroimidazo[1,2-a]pyridin-3-yl)-6-(piperazin-1-yl)pyrimidin-4-ylamino)piperidin-1-yl)-3-oxopropanenitrile;
(R)-3-(3-(2-(6-fluoroimidazo[1,2-a]pyridin-3-yl)-6-(4-(methylsulfonyl)piperazin-1-yl)pyrimidin-4-ylamino)piperidin-1-yl)-3-oxopropanenitrile;
(R)-3-(3-(2-(6-fluoroimidazo[1,2-a]pyridin-3-yl)-6-morpholinopyrimidin-4-ylamino)piperidin-1-yl)-3-oxopropanenitrile;
(R)-3-(3-(6-(dimethylamino)-2-(6-fluoroimidazo[1,2-a]pyridin-3-yl)pyrimidin-4-ylamino)piperidin-1-yl)-3-oxopropanenitrile;
(R)-3-(3-(2-(6-fluoroimidazo[1,2-a]pyridin-3-yl)-6-(2-morpholinoethylamino)pyrimidin-4-ylamino)piperidin-1-yl)-3-oxopropanenitrile;
(R)-3-(3-(2-(6-fluoroimidazo[1,2-a]pyridin-3-yl)-6-(2-methoxyethylamino)pyrimidin-4-ylamino)piperidin-1-yl)-3-oxopropanenitrile;
(S)-1-(6-((R)-1-(2-cyanoacetyl)piperidin-3-ylamino)-2-(6-fluoroimidazo[1,2-a]pyridin-3-yl)pyrimidin-4-yl)pyrrolidine-2-carboxylic acid;
(R)-3-(3-(2-(6-fluoroimidazo[1,2-a]pyridin-3-yl)-6-methoxypyrimidin-4-ylamino)piperidin-1-yl)-3-oxopropanenitrile;
(R)-3-(3-(2-(6-fluoroimidazo[1,2-a]pyridin-3-yl)-6-(2-(pyrrolidin-1-yl)ethoxy)pyrimidin-4-ylamino)piperidin-1-yl)-3-oxopropanenitrile;
(R)-3-(3-(2-(6-fluoroimidazo[1,2-a]pyridin-3-yl)-6-(2-morpholinoethoxy)pyrimidin-4-ylamino)piperidin-1-yl)-3-oxopropanenitrile;
(R)-3-(3-(2-(6-fluoroimidazo[1,2-a]pyridin-3-yl)-6-(2-methoxyethoxy)pyrimidin-4-ylamino)piperidin-1-yl)-3-oxopropanenitrile,
(R)-6-(1-(2-cyanoacetyl)piperidin-3-ylamino)-2-(6-fluoroimidazo[1,2-a]pyridin-3-yl)pyrimidine-4-carbonitrile;
(R)-3-(3-(2-(6-fluoroimidazo[1,2-a]pyridin-3-yl)-6-(2H-tetrazol-5-yl)pyrimidin-4-ylamino)piperidin-1-yl)-3-oxopropanenitrile;
(R)-3-(3-(2-(6-fluoroimidazo[1,2-a]pyridin-3-yl)-6-(1-methyl-1H-tetrazol-5-yl)pyrimidin-4-ylamino)piperidin-1-yl)-3-oxopropanenitrile;
(R)-3-(3-(2-(6-fluoroimidazo[1,2-a]pyridin-3-yl)-6-(2-methyl-2H-tetrazol-5-yl)pyrimidin-4-ylamino)piperidin-1-yl)-3-oxopropanenitrile;
(R)-6-(1-(2-cyanoacetyl)piperidin-3-ylamino)-2-(6-fluoroimidazo[1,2-a]pyridin-3-yl)pyrimidine-4-carboxylic acid;
(R)-6-(1-(2-cyanoacetyl)piperidin-3-ylamino)-2-(6-fluoroimidazo[1,2-a]pyridin-3-yl)pyrimidine-4-carboxamide;
(R)-3-(3-(6-amino-2-(6-fluoroimidazo[1,2-a]pyridin-3-yl)pyrimidin-4-ylamino)piperidin-1-yl)-3-oxopropanenitrile;
(R)-3-(3-(5-fluoro-2-(6-fluoroimidazo[1,2-a]pyridin-3-yl)-6-morpholinopyrimidin-4-ylamino)piperidin-1-yl)-3-oxopropanenitrile;
(R)-1-((R)-3-(5-fluoro-2-(6-fluoroimidazo[1,2-a]pyridin-3-yl)-6-morpholinopyrimidin-4-ylamino)piperidin-1-yl)-2,3-dihydroxypropan-1-one;
(R)-3-(3-(2-(6-fluoroimidazo[1,2-a]pyridin-3-yl)-6-methoxy-5-(trifluoromethyl)pyrimidin-4-ylamino)piperidin-1-yl)-3-oxopropanenitrile;
or a pharmaceutically acceptable salt, or solvate or N-oxides or stereoisomer thereof.

25. The method according to claim 1, wherein the treatment is by inhibition of Janus Kinases.

26. The method according to claim 1, wherein the pathological condition or disease is selected from myeloproliferative disorders, leukemia, lymphoid malignancies and solid tumors; bone marrow and organ transplant rejection; and immune-mediated diseases, or wherein the pathological condition or disease is selected from inflammatory diseases.

27. The method according to claim 26, wherein the pathological condition or disease is selected from myeloproliferative disorders, leukemia, lymphoid malignancies and solid tumors and wherein the treatment is by inhibition of Janus kinases.

28. The method according to claim 26, wherein the pathological condition or disease is selected from bone marrow and organ transplant rejection; and immune-mediated diseases, or wherein the pathological condition or disease is selected from inflammatory diseases.

29. The method according to claim 1, wherein the pathological condition or disease is selected from rheumatoid arthritis, multiple sclerosis, inflammatory bowel disease, dry eye, uveitis, allergic conjunctivitis, allergic rhinitis, asthma, chronic obstructive pulmonary disease (COPD), atopic dermatitis and psoriasis.

30. (canceled)

31. (canceled)

32. A method of inhibiting Janus Kinases in a subject in need thereof, comprising administering to said subject a therapeutically effective amount of a compound of formula (I) as claimed in claim 1, or a pharmaceutical composition comprising a compound of formula (I) in association with a pharmaceutically acceptable diluent or carrier.

33. A compound of formula (I), or a pharmaceutically acceptable salt, or solvate, or N-oxide, or stereoisomer or deuterated derivative thereof: 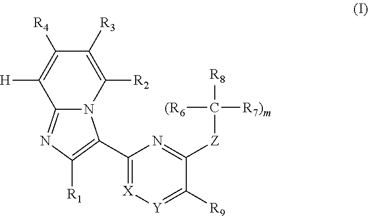
wherein m, X, Y and R₁ to R₉ are as defined in claim 1, other than:
2-Pyrazinamine, 6-imidazo[1,2-a]pyridin-3-yl-N-(3R)-3-piperidinyl-, hydrochloride;
2-Pyrazinamine, 6-imidazo[1,2-a]pyridin-3-yl-N-(3S)-3-piperidinyl-;
2-Pyrazinamine, 6-(6-chloroimidazo[1,2-a]pyridin-3-yl)-N-(3R)-3-piperidinyl-;
2-Pyrazinamine, 6-(6-methoxyimidazo[1,2-a]pyridin-3-yl)-N-(3R)-3-piperidinyl-;
2-Pyrazinamine, 6-imidazo[1,2-a]pyridin-3-yl-N-(3R)-3-pyrrolidinyl-;
2-Pyrazinamine, N-(4,4-difluoro-3-piperidinyl)-6-imidazo[1,2-a]pyridin-3-yl-, hydrochloride;
2-Pyrazinamine, 6-imidazo[1,2-a]pyridin-3-yl-N-[(3R,4R)-4-methyl-3-pyrrolidinyl]-;
2-Pyrazinamine, N-(3R)-3-pyrrolidinyl-6-[6-(trifluoromethyl)imidazo[1,2-a]pyridin-3-yl]-;
Imidazo[1,2-a]pyridine-6-methanol, α,α-dimethyl-3[6-[[(3R,4R)-4-methyl-3-pyrrolidinyl]amino]-2-pyrazinyl]-;
2-Pyrazinamine, 6-imidazo[1,2-a]pyridin-3-yl-3-methyl-N-(3R)-3-piperidinyl-;
1-azabicyclo[2.2.2]octan-3-amine, N-[6-(7-methoxyimidazo[1,2-a]pyridin-3-yl)-2-pyrazinyl]-2-methyl-, (2R,3R)-;
3-Imidazo[1,2-a]pyridin-3-yl-5-[(3R)-3-piperidinylamino]-2-pyrazinecarbonitrile, formate salt;
Imidazo[1,2-a]pyridine-7-carbonitrile, 3-[6-[(3R)-3-piperidinylamino]-2-pyrazinyl]-;
2-Pyrazinamine, N-[(3R,5S)-5-(difluoromethyl)-3-pyrrolidinyl]-6-imidazo[1,2-a]pyridin-3-yl-;
2-Pyrazinamine, N-[(3S,4R)-4-fluoro-3-piperidinyl]-6-imidazo[1,2-a]pyridin-3-yl-;
2-Pyrazinamine, N-[(3S,4S)-4-fluoro-3-piperidinyl]-6-imidazo[1,2-a]pyridin-3-yl-;
2-Pyrazinamine, 6-imidazo[1,2-a]pyridin-3-yl-N-[(3S,4S)-4-methoxy-3-piperidinyl]-;
6-imidazo[1,2-a]pyridin-3-yl-N-[(3R,6S)-6-methyl-3-piperidinyl]-2-pyrazinamine, formate salt;
6-imidazo[1,2-a]pyridin-3-yl-N-[(3R,6R)-6-methyl-3-piperidnyl]-2-pyrazinamine, formate salt;
2-Pyrazinamine, 6-[7-[2-(1-methylethoxy)ethoxy]imidazo[1,2-a]pyridin-3-yl]-N-(3R)-3-piperidinyl-;
2-Pyrazinamine, N-[(3S,4S)-4-ethoxy-3-piperidinyl]-6-imidazo[1,2-a]pyridin-3-yl-;
N-[(3S,4S)-4-ethoxy-piperidin-3-yl]-6-(imidazo[1,2a]pyridin-3-yl)pyrazin-2-amine;
2-Pyrazinamine, 6-imidazo[1,2-a]pyridin-3-yl-3-methoxy-N-(3R)-3-piperidinyl-, 2,2,2-trifluoroacetate;
2-Pyrazinamine, 6-(7-methoxyimidazo[1,2-a]pyridin-3-yl)-N-(3R)-3-piperidinyl-;
2-Pyrazinamine, 6-(7-methylimidazo[1,2-a]pyridin-3-yl)-N-(3R)-3-piperidinyl-;
2-Pyrazinamine, N-(3R)-3-piperidinyl-6-[6-(trifluoromethyl)imidazo[1,2-a]pyridin-3-yl]-;
2-Pyrazinamine, N-(3R)-3-piperidinyl-6-[7-(trifluoromethyl)imidazo[1,2-a]pyridin-3-yl]-;
2-Pyrazinamine, 6-(7-ethylimidazo[1,2-a]pyridin-3-yl)-N-(3R)-3-piperidinyl-;
2-Pyrazinamine, 6-[6-(1-methylethyl)imidazo[1,2-a]pyridin-3-yl]-N-(3R)-3-piperidinyl-;
2-Pyrazinamine, 6-(7-chloroimidazo[1,2-a]pyridin-3-yl)-N-(3R)-3-piperidinyl-;
2-Pyrazinamine, N-[(3R,4R)-4-fluoro-3-pyrrolidinyl]-6-imidazo[1,2-a]pyridin-3-yl-;
2-Pyrazinamine, 6-[5-[(3-methyl-3-oxetanyl)methoxy]-1H-benzimidazol-1-yl]-N-(3R)-3-piperidinyl-;
2-Pyrazinamine, 6-imidazo[1,2-a]pyridin-3-yl-N-(3S)-3-pyrrolidinyl-;
2-Pyrazinamine, 6-(6-methylimidazo[1,2-a]pyridin-3-yl)-N-(3R)-3-pyrrolidinyl-;
2-Pyrazinamine, 6-(7-methylimidazo[1,2-a]pyridin-3-yl)-N-(3R)-3-pyrrolidinyl-,
2-Pyrazinamine, 6-(7-methoxyimidazo[1,2-a]pyridin-3-yl)-N-(3R)-3-pyrrolidinyl-;
2-Pyrazinamine, N-(3R)-3-pyrrolidinyl-6-[7-(trifluoromethyl)imidazo[1,2-a]pyridin-3-yl]-;
2-Pyrazinamine, 6-imidazo[1,2-a]pyridin-3-yl-N-(3-methyl-3-piperidinyl)-;
2-Pyrazinamine, 6-(6-methoxyimidazo[1,2-a]pyridin-3-yl)-N-(3R)-3-pyrrolidinyl-;
2-Pyrazinamine, 6-(7-ethylimidazo[1,2-a]pyridin-3-yl)-N-(3R)-3-pyrrolidinyl-;
2-Pyrazinamine, 6-[6-(1-methylethyl)imidazo[1,2-a]pyridin-3-yl]-N-(3R)-3-pyrrolidinyl-;
2-Pyrazinamine, 6-(5-methylimidazo[1,2-a]pyridin-3-yl)-N-(3R)-3-pyrrolidinyl-;
2-Pyrazinamine, 6-(7-chloroimidazo[1,2-a]pyridin-3-yl)-N-[(3S,4S)-4-fluoro-3-pyrrolidinyl]-;
2-Pyrazinamine, N-[(3R,5S)-5-(fluoromethyl)-3-pyrrolidinyl]-6-imidazo[1,2-a]pyridin-3-yl-;
2-Pyrazinamine, N-[(3R,5S)-5-(fluoromethyl)-3-pyrrolidinyl]-6-[7-(trifluoromethyl)imidazo[1,2-a]pyridin-3-yl]-;
3-{6-[(3R)-piperidin-3-ylamino]pyrazin-2-yl}imidazo[1,2a]pyridine-7-carboxamide;
2-Pyrazinamine, 6-(6-fluoroimidazo[1,2-a]pyridin-3-yl)-N-(3R)-3-piperidinyl-;
2-Pyrazinamine, 6-(6-fluoroimidazo[1,2-a]pyridin-3-yl)-N-[(3R,6R)-6-methyl-3-piperidinyl]-;
2-Pyrazinamine, N-[(3R,5S)-5-(difluoromethyl)-3-pyrrolidinyl]-6[7-(trifluoromethyl)imidazo[1,2-a]pyridin-3-yl];
2-Pyrazinamine, 6-(6-fluoroimidazo[1,2-a]pyridin-3-yl)-N-[(3S,4R)-4-fluoro-3-pyrrolidinyl]-;
2-Pyrazinamine, 6-(6-fluoroimidazo[1,2-a]pyridin-3-yl)-N-(3R)-3-pyrrolidinyl-;
2-Pyrazinamine, 6-(6-fluoroimidazo[1,2-a]pyridin-3-yl)-N-[(3S,4S)-4-fluoro-3-pyrrolidinyl]-;
2-Pyrazinamine, 6-(6-fluoroimidazo[1,2-a]pyridin-3-yl)-N-[(3R,6S)-6-methyl-3-piperidinyl]-;
2-Pyrazinamine, 6-(6-fluoroimidazo[1,2-a]pyridin-3-yl)-N-[(2R,3R)-2-methyl-3-piperidinyl]-;
2-Pyrazinamine, 6-(6-fluoroimidazo[1,2-a]pyridin-3-yl)-N-[(2S,3R)-2-methyl-3-piperidinyl]-;
6-Imidazo[1,2-a]pyridin-3-yl-N-(3-methylpyrrolidin-3-yl)pyrazin-2-amine;
2-Pyrazinamine, 6-imidazo[1,2-a]pyridin-3-yl-N-[(2S,3R)-2-methyl-3-piperidinyl]-;
2-Pyrazinamine, 6-(7-chloroimidazo[1,2-a]pyridin-3-yl)-N-[(2R,3R)-2-methyl-3-piperidinyl]-;
2-Pyrazinamine, 6-(7-chloroimidazo[1,2-a]pyridin-3-yl)-N-[(2S,3R)-2-methyl-3-piperidinyl]-;
2-Pyrazinamine, 6-imidazo[1,2-a]pyridin-3-yl-N-[(2R,3R)-2-methyl-3-piperidinyl]-;
2-Pyrazinamine, N-(3R)-3-piperidinyl-6-[7-(2,2,2-trifluoro-1-methylethoxy)imidazo[1,2-a]pyridin-3-yl]-;
2-Pyrazinamine, 6-[7-[1-(methoxymethyl)propoxy]imidazo[1,2-a]pyridin-3-yl]-N-(3R)-3-piperidinyl-;
2-azabicyclo[2.2.1]heptan-6-amine, N-(6-imidazo[1,2-a]pyridin-3-yl-2-pyrazinyl)-, (1S,4R,6R)-;
2-azabicyclo[2.2.1]heptan-6-amine, N-[6-(7-methoxyimidazo[1,2-a]pyridin-3-yl)-2-pyrazinyl]-, (1S,4R,6R)-;
2-azabicyclo[2.2.1]heptan-6-amine, N-[6-(6-fluoroimidazo[1,2-a]pyridin-3-yl)-2-pyrazinyl]-, (1S,4R,6R)-;
2-Pyrazinamine, 6-(7-chloroimidazo[1,2-a]pyridin-3-yl)-N-[(3R,4R)-4-methyl-3-pyrrolidinyl]-;
2-Pyrazinamine, N-[(3R,4R)-4-(fluoromethyl)-3-pyrrolidinyl]-6-imidazo[1,2-a]pyridin-3-yl-;
2-Pyrazinamine, 6-imidazo[1,2-a]pyridin-3-yl-N-[(2S,3R)-2-methyl-3-piperidinyl]-,
2-Pyrazinamine, 6-imidazo[1,2-a]pyridin-3-yl-N-[(2R,3S)-2-methyl-3-piperidinyl]-;
2-Pyrazinamine, N-(3R)-3-pyrrolidinyl-6-[7-(2,2,2-trifluoro-1-methylethoxy)imidazo[1,2-a]pyridin-3-yl]-;
Imidazo[1,2-a]pyridin-7-ol, 3-[6-[(3R)-3-piperidinylamino]-2-pyrazinyl]-;
2-Pyrazinamine, 6-imidazo[1,2-a]pyridin-3-yl-N-[(2R,3R)-2-methyl-3-piperidinyl]-;
2-Pyrazinamine, 6-imidazo[1,2-a]pyridin-3-yl-N-[(2S,3S)-2-methyl-3-piperidinyl]-;
2-Pyrazinamine, 6-(6-fluoroimidazo[1,2-a]pyridin-3-yl)-N-[(3S,4S)-4-methyl-3-pyrrolidinyl]-;
2-Pyrazinamine, 6-imidazo[1,2-a]pyridin-3-yl-N-[(3R,5R)-5-methyl-3-pyrrolidinyl]-;
2-Pyrazinamine, 6-[7-(3,3-dimethylbutoxy)imidazo[1,2-a]pyridin-3-yl]-N-(3R)-3-piperidinyl-;
Imidazo[1,2-a]pyridine-6-methanol, 3[6-[(3R)-3-piperidinylamino]-2-pyrazinyl]-;
2-Pyrazinamine, 6-(7-methoxyimidazo[1,2-a]pyridin-3-yl)-N-[(3R,4R)-4-phenyl-3-pyrrolidinyl]-;
2-Pyrazinamine, 6-(2-methylimidazo[1,2-a]pyridin-3-yl)-N-(3R)-3-piperidinyl-;
2-Pyrazinamine, 6-[7-[(3-methyl-3-oxetanyl)methoxy]imidazo[1,2-a]pyridin-3-yl]-N-(3R)-3-pyrrolidinyl-;
2-Pyrazinamine, 6-[7-[(3-methyl-3-oxetanyl)methoxy]imidazo[1,2-a]pyridin-3-yl]-N-(3R)-3-piperidinyl-;
2-Pyrazinamine, 6-imidazo[1,2-a]pyridin-3-yl-N-[(3R,5R)-5-methoxy-3-piperidinyl]-;
2-Pyrazinamine, 6-imidazo[1,2-a]pyridin-3-yl-N-[(3R,5S)-5-(methoxymethyl)-3-pyrrolidinyl]-;
2-Pyrazinamine, 6-imidazo[1,2-a]pyridin-3-yl-N-[(3R,5S)-5-[(1-methylethoxy)methyl]-3-pyrrolidinyl]-;
2-azabicyclo[2.2.1]heptan-6-amine, N-(6-imidazo[1,2-a]pyridin-3-yl-2-pyrazinyl)-;
Imidazo[1,2-a]pyridine-7-carbonitrile, 3-[6-[(1S,4R,6R)-2-azabicyclo[2.2.1]hept-6-ylamino]-2-pyrazinyl]-;
2-Pyrazinamine, 6-(6-fluoroimidazo[1,2-a]pyridin-3-yl)-N-[(2R,3S)-2-methyl-3-piperidinyl]-;
2-Pyrazinamine, 6-(6-fluoroimidazo[1,2-a]pyridin-3-yl)-N-[(2R,3R)-2-methyl-3-piperidinyl]-;
2-Pyrazinamine, 6-(6-fluoroimidazo[1,2-a]pyridin-3-yl)-N-[(2S,3R)-2-methyl-3-piperidinyl]-;
Imidazo[1,2-a]pyridine-7-carbonitrile, 3-[6-[(3R)-3-pyrrolidinylamino]-2-pyrazinyl]-;
Imidazo[1,2-a]pyridine-7-carbonitrile, 3-[6-[[(3R,4R)-4-methyl-3-pyrrolidinyl]amino]-2-pyrazinyl]-;
Imidazo[1,2-a]pyridine-7-carbonitrile, 3-[6-[[(3R,5S)-5-(methoxymethyl)-3-pyrrolidinyl]amino]-2-pyrazinyl]-;
Imidazo[1,2-a]pyridine-7-carbonitrile, 3[6-[[(3R,5R)-5-methyl-3-pyrrolidinyl]amino]-2-pyrazinyl]-;
2-Pyrazinamine, 6-imidazo[1,2-a]pyridin-3-yl-N-[(3S,4R)-4-methoxy-3-pyrrolidinyl]-;
Imidazo[1,2-a]pyridine-7-carboxamide, 3-[6-[[(3R,4R)-4-methyl-3-pyrrolidinyl]amino]-2-pyrazinyl]-;
2-Pyrazinamine, 6-imidazo[1,2-a]pyridin-3-yl-N-[(3R,5S)-5-(propoxymethyl)-3-pyrrolidinyl]-;
2-Pyrazinamine, N-[(3R,5S)-5-(ethoxymethyl)-3-pyrrolidinyl]-6-imidazo[1,2-a]pyridin-3-yl-;
2-Pyrazinamine, 6-imidazo[1,2-a]pyridin-3-yl-N-[(3R,4R)-4-(1-methylethyl)-3-pyrrolidinyl]-;
2-Pyrazinamine, 6-imidazo[1,2-a]pyridin-3-yl-N-[(3S,4R)-4-(2,2,2-trifluoroethoxy)-3-pyrrolidinyl]-;
2-Pyrazinamine, N-[(3R,4R)-4-ethyl-3-pyrrolidinyl]-6-imidazo[1,2-a]pyridin-3-yl-;
2-Pyrazinamine, N-[(3R,4R)-4-cyclopropyl-3-pyrrolidinyl]-6-imidazo[1,2-a]pyridin-3-yl-;
3-Piperidinol, 5-[(6-imidazo[1,2-a]pyridin-3-yl-2-pyrazinyl)amino]-, (3R,5R)-;
2-Pyrazinamine, 6-imidazo[1,2-a]pyridin-3-yl-N-[(3R,4R)-4-methyl-3-piperidinyl]-;
2-Pyrazinamine, 6-(7-chloroimidazo[1,2-a]pyridin-3-yl)-N-[(3R,4R)-4-methyl-3-piperidinyl]-;
2-Pyrazinamine, 6-(8-methylimidazo[1,2-a]pyridin-3-yl)-N-(3R)-3-piperidinyl-;
2-Pyrazinamine, 6-[7-(1-methyl-1H-pyrazol-4-yl)Imidazo[1,2-a]pyridin-3-yl]-N-[(3R,4R)-4-methyl-3-pyrrolidinyl]-;
2-Pyrazinamine, N-[(3R,4R)-4-cyclopentyl-3-pyrrolidinyl]-6-(7-methoxyimidazo[1,2-a]pyridin-3-yl)-;
2-Pyrazinamine, N-[(3R,4R)-4-cyclopropyl-3-pyrrolidinyl]-6-(7-methoxyimidazo[1,2-a]pyridin-3-yl)-;
2-Pyrazinamine, 6-imidazo[1,2-a]pyridin-3-yl-3-methoxy-N-(3R)-3-pyrrolidinyl-;
2-Pyrazinamine, 6-[7-[2-(1-methylethoxy)ethoxy]imidazo[1,2-a]pyridin-3-yl]-N-(3R)-3-pyrrolidinyl-;
2-Pyrazinamine, 6-imidazo[1,2-a]pyridin-3-yl-3-methyl-N-(3R)-3-pyrrolidinyl-;
2-Pyrazinamine, 6-(6-chloroimidazo[1,2-a]pyridin-3-yl)-N-[(2S,3R)-2-methyl-3-piperidinyl]-;
2-Pyrazinamine, 6-(6-chloroimidazo[1,2-a]pyridin-3-yl)-N-[(2R,3R)-2-methyl-3-piperidinyl]-;
2-Pyrazinamine, N-[(2S,3R)-2-methyl-3-piperidinyl]-6-[6-(trifluoromethyl)imidazo[1,2-a]pyridin-3-yl]-;
2-Pyrazinamine, N-[(2R,3R)-2-methyl-3-piperidinyl]-6-[6-(trifluoromethyl)imidazo[1,2-a]pyridin-3-yl]-;
2-Pyrazinamine, 6-(6-chloroimidazo[1,2-a]pyridin-3-yl)-N-[(3R,4R)-4-methyl-3-pyrrolidinyl]-;
2-Pyrazinamine, 6-(6-chloroimidazo[1,2-a]pyridin-3-yl)-N-(4,4-difluoro-3-piperidinyl)-;
2-Pyrazinamine, N-(4,4-difluoro-3-piperidinyl)-6-[6-(trifluoromethyl)imidazo[1,2-a]pyridin-3-yl]-;
Imidazo[1,2-a]pyridine-6-methanol, α,α-dimethyl-3-[6-[(3R)-3-piperidinylamino]-2-pyrazinyl]-;
Imidazo[1,2-a]pyridin-6-amine, 3-[6-[(3R)-3-piperidinylamino]-2-pyrazinyl]-;
2-Pyrazinamine, 6-(7-fluoroimidazo[1,2-a]pyridin-3-yl)-N-(3R)-3-piperidinyl-;
2-Pyrazinamine, 6-(7-fluoroimidazo[1,2-a]pyridin-3-yl)-N-[(3R,5S)-5-(methoxymethyl)-3-pyrrolidinyl]-;
2-Pyrrolidinemethanol, 4-[(6-imidazo[1,2-a]pyridin-3-yl-2-pyrazinyl)amino]-, (2S,4R)-;
2-Pyrazinamine, N-[(3R,5S)-5-(methoxymethyl)-3-pyrrolidinyl]-6-(7-methylimidazo[1,2-a]pyridin-3-yl)-;
2-Pyrazinamine, N-[(3R,5S)-5-[(1-methylethoxy)methyl]-3-pyrrolidinyl]-6-(7-methylimidazo[1,2-a]pyridin-3-yl)-;
2-Pyrazinamine, N-[(3R,5S)-5-(ethoxymethyl)-3-pyrrolidinyl]-6-(7-methylimidazo[1,2-a]pyridin-3-yl)-;
2-Pyrazinamine, 6-(7-methoxyimidazo[1,2-a]pyridin-3-yl)-N-[(3R,5S)-5-[(1-methylethoxy)methyl]-3-pyrrolidinyl]-;
2-Pyrazinamine, N-[(3R,5S)-5-(ethoxymethyl)-3-pyrrolidinyl]-6-(7-methoxyimidazo[1,2-a]pyridin-3-yl)-;
2-Pyrazinamine, 6-(7-chloroimidazo[1,2-a]pyridin-3-yl)-N-[(3R,5S)-5-(methoxymethyl)-3-pyrrolidinyl]-;
2-Pyrrolidinemethanol, 4-[[6-(7-chloroimidazo[1,2-a]pyridin-3-yl)-2-pyrazinyl]amino]-, (2S,4R)-;
2-Pyrazinamine, 6-(6-chloroimidazo[1,2-a]pyridin-3-yl)-N-[(3R,5S)-5-(methoxymethyl)-3-pyrrolidinyl]-;
2-Pyrazinamine, 6-imidazo[1,2-a]pyridin-3-yl-5-methyl-N-(3R)-3-piperidinyl-;
2-Pyrazinamine, N-[(3R)-4,4-difluoro-3-piperidinyl]-6-imidazo[1,2-a]pyridin-3-yl-;
2-Pyrazinamine, N-[(3S)-4,4-difluoro-3-piperidinyl]-6-imidazo[1,2-a]pyridin-3-yl-;
2-Pyrazinamine, 6-imidazo[1,2-a]pyridin-3-yl-N-[(3S,5S)-5-methoxy-3-piperidinyl]-;
2-Pyrazinamine, 6-imidazo[1,2-a]pyridin-3-yl-N-[(3S,4S)-4-methoxy-3-piperidinyl]-;
2-Pyrazinamine, 6-imidazo[1,2-a]pyridin-3-yl-N-[(3R,4R)-4-methoxy-3-piperidinyl]-, hydrochloride;
Imidazo[1,2-a]pyridine-7-carbonitrile, 3-[6-[[(2R,3R)-2-methyl-3-piperidinyl]amino]-2-pyrazinyl]-;
Imidazo[1,2-a]pyridine-6-carbonitrile, 3[6-[[(3R,4R)-4-cyclopropyl-3-pyrrolidinyl]amino]-2-pyrazinyl]-;
Imidazo[1,2-a]pyridine-7-carbonitrile, 3[6-[[(3R,4R)-4-cyclopropyl-3-pyrrolidinyl]amino]-2-pyrazinyl]-;
1-azabicyclo[2.2.2]octan-3-amine, N-[6-(7-methoxyimidazo[1,2-a]pyridin-3-yl)-2-pyrazinyl]-2-methyl-, (2S,3S)-;
2-Pyrazinamine, 6-(6-fluoroimidazo[1,2-a]pyridin-3-yl)-N-[(3R,5S)-5-(methoxymethyl)-3-pyrrolidinyl]-;
2-Pyrazinecarbonitrile, 3-imidazo[1,2-a]pyridin-3-yl-5-[(3R)-3-pyrrolidinylamino]-;
2-Pyrazinamine, N-(3S)-3-pyrrolidinyl-6-[6-(trifluoromethyl)imidazo[1,2-a]pyridin-3-yl]-;
Ethanol, 2-[[3-[6-[(3R)-3-pyrrolidinylamino]-2-pyrazinyl]imidazo[1,2-a]pyridin-7-yl]oxy]-
1-Propanol, 2-[[3-[6-[(3R)-3-pyrrolidinylamino]-2-pyrazinyl]imidazo[1,2-a]pyridin-7-yl]oxy]-, (2S);
1-Propanol, 2-[[3-[6-[(3R)-3-piperidinylamino]-2-pyrazinyl]imidazo[1,2-a]pyridin-7-yl]oxy]-, (2S)-;
2-Propanol, 1-[[3-[6-[(3R)-3-piperidinylamino]-2-pyrazinyl]imidazo[1,2-a]pyridin-7-yl]oxy]-, (2S)-;
1-Propanol, 2-[[3-[6-[(3R)-3-pyrrolidinylamino]-2-pyrazinyl]imidazo[1,2-a]pyridin-7-yl]oxy]-, (2R)-;
Ethanol, 2-[[3-[6-[(3R)-3-piperidinylamino]-2-pyrazinyl]imidazo[1,2-a]pyridin-7-yl]oxy]-;
2-Pyrazinamine, 6-imidazo[1,2-a]pyridin-3-yl-5-methyl-N-(3R)-3-pyrrolidinyl-;
1-Piperidinecarboxylic acid, 3-[[6-(6-chloroimidazo[1,2-a]pyridin-3-yl)-2-pyrazinyl]amino]-, 1,1-dimethylethyl ester, (3R)-;
1-Piperidinecarboxylic acid, 3-[[6-(6-methoxyimidazo[1,2-a]pyridin-3-yl)-2-pyrazinyl]amino]-, 1,1-dimethylethyl ester, (3R)-;
1-Piperidinecarboxylic acid, 3-[[6-[7-(aminocarbonyl)imidazo[1,2-a]pyridin-3-yl]-2-pyrazinyl]amino]-, 1,1-dimethylethyl ester, (3R)-;
1-Piperidinecarboxylic acid, 4-ethoxy-3-[(6-imidazo[1,2-a]pyridin-3-yl-2-pyrazinyl)amino]-, phenylmethyl ester, (3R,4R)-;
1-Piperidinecarboxylic acid, 3-[(6-imidazo[1,2-a]pyridin-3-yl-2-pyrazinyl)amino]-, 1,1-dimethylethyl ester, (3R)-;
1-Piperidinecarboxylic acid, 3-[(6-imidazo[1,2-a]pyridin-3-yl-2-pyrazinyl)amino]-, 1,1-dimethylethyl ester, (3S)-;
1-Pyrrolidinecarboxylic acid, 3-[(6-imidazo[1,2-a]pyridin-3-yl-2-pyrazinyl)amino]-, 1,1-dimethylethyl ester, (3R)-;
1-Pyrrolidinecarboxylic acid, 3-[(6-imidazo[1,2-a]pyridin-3-yl-2-pyrazinyl)amino]-4-methyl-, 1,1-dimethylethyl ester, (3R,4R)-;
1-Pyrrolidinecarboxylic acid, 3[[6[6-(trifluoromethyl)imidazo[1,2-a]pyridin-3-yl]-2-pyrazinyl]amino]-, 1,1-dimethylethyl ester, (3R)-;
1-Pyrrolidinecarboxylic acid, 3-[[6-[6-(1-hydroxy-1-methylethyl)imidazo[1,2-a]pyridin-3-yl]-2-pyrazinyl]amino]-4-methyl-, 1,1-dimethylethyl ester, (3R,4R)-;
1-Piperidinecarboxylic acid, 3-[(6-imidazo[1,2-a]pyridin-3-yl-3-methyl-2-pyrazinyl)amino]-, 1,1-dimethylethyl ester, (3R)-;
1-Piperidinecarboxylic acid, 3-[(5-cyano-6-imidazo[1,2-a]pyridin-3-yl-2-pyrazinyl)amino]-, 1,1-dimethylethyl ester, (3R)-;
Imidazo[1,2-a]pyridine-7-carboxylic acid, 3-[6-[[(3R)-1-[(1,1-dimethylethoxy)carbonyl]-3-piperidinyl]amino]-2-pyrazinyl]-, methyl ester;
1-Pyrrolidinecarboxylic acid, 2-(difluoromethyl)-4-[(6-imidazo[1,2-a]pyridin-3-yl-2-pyrazinyl)amino]-, 1,1-dimethylethyl ester, (2S,4R)-;
1-Piperidinecarboxylic acid, 4-fluoro-3-[(6-imidazo[1,2-a]pyridin-3-yl-2-pyrazinyl)amino]-, phenylmethyl ester, (3R,4S)-;
1-Piperidinecarboxylic acid, 4-fluoro-3-[(6-imidazo[1,2-a]pyridin-3-yl-2-pyrazinyl)amino]-, phenylmethyl ester, (3R,4R)-;
1-Piperidinecarboxylic acid, 3-[(6-imidazo[1,2-a]pyridin-3-yl-2-pyrazinyl)amino]-4-methoxy-, phenylmethyl ester, (3R,4R)-;
1-Piperidinecarboxylic acid, 5-[(6-imidazo[1,2-a]pyridin-3-yl-2-pyrazinyl)amino]-2-methyl-, phenylmethyl ester, (2S,5R)—;
1-Piperidinecarboxylic acid, 5-[(6-imidazo[1,2-a]pyridin-3-yl-2-pyrazinyl)amino]-2-methyl-, phenylmethyl ester, (2R,5R)-;
1-Piperidinecarboxylic acid, 3-[[6-[7-[2-(1-methylethoxy)ethoxy]imidazo[1,2-a]pyridin-3-yl]-2-pyrazinyl]amino]-, 1,1-dimethylethyl ester, (3R)-; and
1-Piperidinecarboxylic acid, 3-[(6-imidazo[1,2-a]pyridin-3-yl-3-methoxy-2-pyrazinyl)amino]-, 1,1-dimethylethyl ester, (3R)-.

34. A compound of formula (I), or a pharmaceutically acceptable salt, or solvate, or N-oxide, or stereoisomer or deuterated derivative thereof: 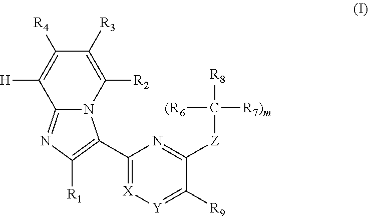
wherein m, X, Y, Z and R₁ to R₉ are as defined in claim 1; and wherein when Y is a nitrogen atom, X is —CR₉, and R₈ is a 5- to 6-membered heterocyclyl containing one nitrogen atom, which nitrogen atom is not bonded to the —Z—(CR₆R₇)ₘ— moiety, and said nitrogen atom is substituted by a substituent other than a tert-butoxycarbonyl or benzyloxycarbonyl.

35. The compound according to claim 34, wherein Y is —CR₉ and X is a nitrogen atom.

36. A pharmaceutical composition comprising the compound according to claim 33 and an acceptable diluent or carrier.

37. A composition comprising
(i) a compound according to claim 33; and
(ii) another compound chosen from:
a) Dyhydrofolate reductase inhibitors,
b) DHODH inhibitors
c) Immunomodulators,
d) Inhibitors of DNA synthesis and repair,
e) anti-alpha 4 integrin antibodies,
f) Alpha 4 integrin antagonists,
g) Corticoids and glucocorticoids,
Fumaric acid esters,
i) anti-TNF alpha antibodies,
j) Soluble TNF alpha receptors,
k) anti-CD20 monoclonal antibodies,
l) anti-CD52,
m) anti-CD25,
n) anti-CD88,
anti-IL12R/IL23R,
p) Calcineurin inhibitors,
q) IMPDH inhibitors,
r) Cannabinoid receptor agonists,
s) Chemokine CCR1 antagonists,
t) Chemokine CCR2 antagonists,
u) NF-kappaB activation inhibitors,
v) S1P receptor agonists,
w) S1P liase inhibitors,
x) Syk inhibitors,
y) PKC inhibitors,
z) M3 antagonist,
aa) Long-acting beta adrenergic agonist,
bb) Vitamin D derivatives,
cc) Phosphosdiesterase IV inhibitors,
dd) p38 Inhibitors,
ee) MEK inhibitors,
ff) PI3Kδγ inhibitors,
Interferons comprising Interferon beta 1a and Interferon beta 1b, and
hh) Interferon alpha,
for simultaneous, separate or sequential use in the treatment of the human or animal body.
